SEQUENCE LISTING (569 sequences)

SEQ ID NO: 1 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 2 - protein, 117 aa, Homo sapiens
VSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 3 - protein, 132 aa, Homo sapiens
QQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 4 - protein, 510 aa, Mus musculus
MPGLGRRAQWLCWWWGLLCSCGPPPLRPPLPVAAAAAGGQLLGAGGSPVRAEQPPPQSSSSGFLYRRLKTHEKREMQKEILSVLGLPHRPRPLHGLQQPQPPVLPPQQQQQQQQQQTAREEPPPGRLKSAPLFMLDLYNALSNDDEEDGASEGVGQEPGSHGGASSSQLRQPSPGAAHSLNRKSLLAPGPGGGASPLTSAQDSAFLNDADMVMSFVNLVEYDKEFSPHQRHHKEFKFNLSQIPEGEAVTAAEFRVYKDCVVGSFKNQTFLISIYQVLQEHQHRDSDLFLLDTRVVWASEEGWLEFDITATSNLWVVTPQHNMGLQLSVVTRDGLHVNPRAAGLVGRDGPYDKQPFMVAFFKVSEVHVRTTRSASSRRRQQSRNRSTQSQDVSRGSGSSDYNGSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 5 - protein, 139 aa, Mus musculus
SASSRRRQQSRNRSTQSQDVSRGSGSSDYNGSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 6 - DNA, 44 bp, Homo sapiens
  source 1..44: mol_type = unassigned DNA
aaaaaaactagtaaatggccccatgtggcccccgccttgtctgc

SEQ ID NO: 7 - DNA, 46 bp, Homo sapiens
  source 1..46: mol_type = unassigned DNA
ttttttctcgagctgtctggctgtcccactgctgggtcttgagctt

SEQ ID NO: 8 - protein, 11 aa, Homo sapiens
TLVHLMNPEYV

SEQ ID NO: 9 - protein, 7 aa, Homo sapiens
HLMNPEY

SEQ ID NO: 10 - protein, 13 aa, Homo sapiens
SASDYNSSELKTA

SEQ ID NO: 11 - protein, 13 aa, Homo sapiens
ELKTACRKHELYV

SEQ ID NO: 12 - protein, 13 aa, Homo sapiens
GSSDYNGSELKTA

SEQ ID NO: 13 - protein, 13 aa, Homo sapiens
ELKTACKKHELYV

SEQ ID NO: 14 - protein, 5 aa, Homo sapiens
ELKTA

SEQ ID NO: 15 - protein, 7 aa, Homo sapiens
QSQDVAR

SEQ ID NO: 16 - protein, 7 aa, Homo sapiens
QSQDVSR

SEQ ID NO: 17 - protein, 30 aa, Homo sapiens
QSRNRSTQSQDVARVSSASDYNSSELKTAC

SEQ ID NO: 18 - protein, 8 aa, Homo sapiens
GYTFTNHA

SEQ ID NO: 19 - protein, 8 aa, Homo sapiens
INAGNGKT

SEQ ID NO: 20 - protein, 11 aa, Homo sapiens
TRRVYGESYDH

SEQ ID NO: 21 - protein, 5 aa, Homo sapiens
NHAIH

SEQ ID NO: 22 - protein, 17 aa, Homo sapiens
WINAGNGKTDYSQNFQG

SEQ ID NO: 23 - protein, 9 aa, Homo sapiens
RVYGESYDH

SEQ ID NO: 24 - protein, 118 aa, Homo sapiens
QVQFVQSGAEVKSPGASVKVSCKASGYTFTNHAIHWVRQAPGQRLEWMGWINAGNGKTDYSQNFQGRVIITRDTSANTAYMSLSSLTSEDTAFYYCTRRVYGESYDHWGQGTLVTVSS

SEQ ID NO: 25 - DNA, 354 bp, Homo sapiens
  source 1..354: mol_type = unassigned DNA
caggtccagtttgtgcagtctggggctgaggtgaagagtcctggggcctctgtgaaggtttcctgcaaggcttctggatacaccttcacaaatcatgctatacattgggtgcgccaggcccccggacaaaggcttgagtggatgggatggatcaacgctggcaatggtaaaactgattattcacagaacttccagggcagagtcatcattaccagggacacatccgcgaacacagcctacatgtccctgagcagcctgacatctgaggacacggctttttattactgtactagaagggtctacggtgaatcgtatgaccactggggccagggaaccctggtcaccgtctcctca

SEQ ID NO: 26 - protein, 445 aa, Homo sapiens
QVQFVQSGAEVKSPGASVKVSCKASGYTFTNHAIHWVRQAPGQRLEWMGWINAGNGKTDYSQNFQGRVIITRDTSANTAYMSLSSLTSEDTAFYYCTRRVYGESYDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 6 aa, Homo sapiens
QGIRNN

SEQ ID NO: 28 - skipped

SEQ ID NO: 29 - protein, 9 aa, Homo sapiens
LQHQIYPWT

SEQ ID NO: 30 - protein, 11 aa, Homo sapiens
RASQGIRNNLG

SEQ ID NO: 31 - protein, 7 aa, Homo sapiens
AASSLQS

SEQ ID NO: 32 - protein, 9 aa, Homo sapiens
LQHQIYPWT

SEQ ID NO: 33 - protein, 107 aa, Homo sapiens
DIQMTQSPSSLSASVGDRVTITCRASQGIRNNLGWYQQKPGKAPKRLIYAASSLQSGVPSRFSGSGSGTEFTLTISSLQPEDVAIYFCLQHQIYPWTFGQGTKVETK

SEQ ID NO: 34 - DNA, 321 bp, Homo sapiens
  source 1..321: mol_type = unassigned DNA
gacatccagatgacccagtctccatcctccctgtctgcatctgtaggagacagagtcaccatcacttgccgggcaagtcagggcattagaaataatttaggctggtatcagcagaaaccagggaaagcccctaagcgcctgatctatgctgcatccagtttgcaaagtggggtcccatcaaggtttagcggcagtggatctgggacagaattcactctcacaatcagcagcctgcagcctgaagatgttgcaatttatttctgtctacaacatcaaatttacccgtggacgttcggccaagggaccaaggtggaaaccaaa

SEQ ID NO: 35 - protein, 214 aa, Homo sapiens
DIQMTQSPSSLSASVGDRVTITCRASQGIRNNLGWYQQKPGKAPKRLIYAASSLQSGVPSRFSGSGSGTEFTLTISSLQPEDVAIYFCLQHQIYPWTFGQGTKVETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 8 aa, Homo sapiens
GYTFTSYA

SEQ ID NO: 37 - protein, 8 aa, Homo sapiens
ISAANGNT

SEQ ID NO: 38 - protein, 11 aa, Homo sapiens
ARRKLWSPFDI

SEQ ID NO: 39 - protein, 5 aa, Homo sapiens
SYALH

SEQ ID NO: 40 - protein, 17 aa, Homo sapiens
WISAANGNTDYSWKFQG

SEQ ID NO: 41 - protein, 9 aa, Homo sapiens
RKLWSPFDI

SEQ ID NO: 42 - protein, 118 aa, Homo sapiens
QVQLVQSGAEVQKPGASVKVSCKASGYTFTSYALHWVRQAPGQRLEWLGWISAANGNTDYSWKFQGRVTLTRDTSANTVYMELNSLTSEDSAVYYCARRKLWSPFDIWGQGTLVTVSS

SEQ ID NO: 43 - DNA, 354 bp, Homo sapiens
  source 1..354: mol_type = unassigned DNA
caggtccaacttgtgcagtctggggctgaggtgcagaagcctggggcctcagtgaaggtttcctgcaaggcttctggatacaccttcacttcatatgctttgcattgggtgcgccaggcccccggacaaaggcttgagtggctgggatggatcagcgctgccaatggtaacacagattattcatggaagttccagggcagagtcacccttaccagggacacatccgcaaacacagtctacatggaactgaacagtctgacatctgaggactcggctgtgtattactgtgcgagaaggaaactatggtctccttttgatatctggggccaagggacattggtcaccgtctcttca

SEQ ID NO: 44 - protein, 445 aa, Homo sapiens
QVQLVQSGAEVQKPGASVKVSCKASGYTFTSYALHWVRQAPGQRLEWLGWISAANGNTDYSWKFQGRVTLTRDTSANTVYMELNSLTSEDSAVYYCARRKLWSPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 7 aa, Homo sapiens
QSLTNSF

SEQ ID NO: 46 - skipped

SEQ ID NO: 47 - protein, 9 aa, Homo sapiens
QYYGTSPWT

SEQ ID NO: 48 - protein, 12 aa, Homo sapiens
WASQSLTNSFLA

SEQ ID NO: 49 - protein, 7 aa, Homo sapiens
GAFSRAT

SEQ ID NO: 50 - protein, 9 aa, Homo sapiens
QYYGTSPWT

SEQ ID NO: 51 - protein, 108 aa, Homo sapiens
EIVLTQSPGTLSLSPGERATLSCWASQSLTNSFLAWYRQKPGQAPRLLISGAFSRATDIPDRISGSGSGTDFTLTINRLEPEDFAVYYCQYYGTSPWTFGQGTKVEIK

SEQ ID NO: 52 - DNA, 324 bp, Homo sapiens
  source 1..324: mol_type = unassigned DNA
gaaattgtgttgacgcagtctccaggcaccctgtctttgtctccaggggaaagagccaccctctcctgttgggccagtcaaagtcttaccaacagtttcttagcctggtaccggcagaaacctggccaggctcccaggctcctcatctctggtgcattcagcagggccactgacatcccagacaggatcagtggcagtggatctgggacagacttcactctcaccatcaacagactggagcctgaagattttgcagtgtattactgtcagtactatggtacctcaccgtggacgttcggccaagggaccaaggtggaaatcaaa

SEQ ID NO: 53 - protein, 215 aa, Homo sapiens
EIVLTQSPGTLSLSPGERATLSCWASQSLTNSFLAWYRQKPGQAPRLLISGAFSRATDIPDRISGSGSGTDFTLTINRLEPEDFAVYYCQYYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 8 aa, Homo sapiens
GYTFTTFA

SEQ ID NO: 55 - protein, 8 aa, Homo sapiens
INPGNVKT

SEQ ID NO: 56 - protein, 11 aa, Homo sapiens
ARRQLWLPFDY

SEQ ID NO: 57 - protein, 5 aa, Homo sapiens
TFAIH

SEQ ID NO: 58 - protein, 17 aa, Homo sapiens
WINPGNVKTDYSQKFQG

SEQ ID NO: 59 - protein, 9 aa, Homo sapiens
RQLWLPFDY

SEQ ID NO: 60 - protein, 118 aa, Homo sapiens
QVHLVQSGAEVKNPGASVKVSCKASGYTFTTFAIHWLRQAPGQRLEWMGWINPGNVKTDYSQKFQGRVTISRDTSATTAYMELSSLRSEDTAVYYCARRQLWLPFDYWGQGTLVTVSS

SEQ ID NO: 61 - DNA, 355 bp, Homo sapiens
  source 1..355: mol_type = unassigned DNA
caggtccaccttgttcagtcaggggcagaggtgaagaaccctggggcctcagtgaaggtctcctgcaaggcttctggatacaccttcactacctttgctattcattggttgcgccaggcccccggacagaggcttgagtggatgggatggatcaaccctggcaatgttaagacagattattcgcagaagttccagggcagagtcaccattagcagggacacatccgcgaccactgcctacatggagctgagcagcctgagatctgaagacacggctgtttattactgtgcgagaagacaattatggttaccctttgactactggggccagggaaccctggtcaccgtctcctcag

SEQ ID NO: 62 - protein, 445 aa, Homo sapiens
QVHLVQSGAEVKNPGASVKVSCKASGYTFTTFAIHWLRQAPGQRLEWMGWINPGNVKTDYSQKFQGRVTISRDTSATTAYMELSSLRSEDTAVYYCARRQLWLPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 7 aa, Homo sapiens
QSISNNF

SEQ ID NO: 64 - skipped

SEQ ID NO: 65 - protein, 9 aa, Homo sapiens
QHYGGSPWT

SEQ ID NO: 66 - protein, 12 aa, Homo sapiens
RASQSISNNFLA

SEQ ID NO: 67 - protein, 7 aa, Homo sapiens
GASSRAT

SEQ ID NO: 68 - protein, 9 aa, Homo sapiens
QHYGGSPWT

SEQ ID NO: 69 - protein, 108 aa, Homo sapiens
EIVLTQSPGTLSLSPGEGATLSCRASQSISNNFLAWYQQKPGQAPRLLIFGASSRATAIPDRFVGSGSGTDFTLTITGLEPEDFAVYHCQHYGGSPWTFGQGTKVEIK

SEQ ID NO: 70 - DNA, 324 bp, Homo sapiens
  source 1..324: mol_type = unassigned DNA
gaaattgtgttgacgcagtctccaggcaccctgtctttgtctccaggggagggagccaccctctcctgcagggccagtcagagtattagcaacaacttcttagcctggtaccaacagaaacctggccaggctcccagactcctcatctttggtgcatccagcagggccactgccatcccagacaggttcgttggcagtgggtctgggacagacttcactctcaccatcaccggactggagcctgaagattttgcagtgtatcactgtcaacactatggtggttcaccttggacgttcggccaagggaccaaggtggaaatcaaa

SEQ ID NO: 71 - protein, 215 aa, Homo sapiens
EIVLTQSPGTLSLSPGEGATLSCRASQSISNNFLAWYQQKPGQAPRLLIFGASSRATAIPDRFVGSGSGTDFTLTITGLEPEDFAVYHCQHYGGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 8 aa, Homo sapiens
GYSFTNYA

SEQ ID NO: 73 - protein, 8 aa, Homo sapiens
IHAGNGKT

SEQ ID NO: 74 - protein, 11 aa, Homo sapiens
ARRQLWLPFDY

SEQ ID NO: 75 - protein, 5 aa, Homo sapiens
NYALH

SEQ ID NO: 76 - protein, 17 aa, Homo sapiens
WIHAGNGKTEYAQKFQD

SEQ ID NO: 77 - protein, 9 aa, Homo sapiens
RQLWLPFDY

SEQ ID NO: 78 - protein, 118 aa, Homo sapiens
QVQLVQSGAEVKKPGASVKVSCKASGYSFTNYALHWVRQAPGQRLEWMGWIHAGNGKTEYAQKFQDRVTISRDISAITVYMELSSLRSEDTAVYYCARRQLWLPFDYWGQGTLVTVSS

SEQ ID NO: 79 - DNA, 354 bp, Homo sapiens
  source 1..354: mol_type = unassigned DNA
caggtccagcttgtgcagtctggggctgaggtgaagaagcctggggcctcagtgaaagtttcctgtaaggcttctgggtacagtttcactaactatgctttacattgggtgcgccaggcccccggacaaagacttgagtggatgggatggatccacgctggtaatggtaagacagaatatgcacagaagttccaggacagagtcaccattagtagggacatatccgcgatcacagtttacatggaactgagcagcctgagatctgaagacacggctgtttattattgtgcgagaagacagttatggttaccctttgactactggggccagggaaccctggtcaccgtctcctca

SEQ ID NO: 80 - protein, 445 aa, Homo sapiens
QVQLVQSGAEVKKPGASVKVSCKASGYSFTNYALHWVRQAPGQRLEWMGWIHAGNGKTEYAQKFQDRVTISRDISAITVYMELSSLRSEDTAVYYCARRQLWLPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 7 aa, Homo sapiens
QIIINRQ

SEQ ID NO: 82 - skipped

SEQ ID NO: 83 - protein, 9 aa, Homo sapiens
QHYGGSPWT

SEQ ID NO: 84 - protein, 12 aa, Homo sapiens
RAGQIIINRQLA

SEQ ID NO: 85 - protein, 7 aa, Homo sapiens
GASNRVT

SEQ ID NO: 86 - protein, 9 aa, Homo sapiens
QHYGGSPWT

SEQ ID NO: 87 - protein, 108 aa, Homo sapiens
EIVLTQSPDTLSLSPGETASFSCRAGQIIINRQLAWYQRRPGQAPRLLIYGASNRVTGIPDRFSGSGSGTDFTLTINRLEPEDFAVYYCQHYGGSPWTFGQGTKVEIK

SEQ ID NO: 88 - DNA, 324 bp, Homo sapiens
  source 1..324: mol_type = unassigned DNA
gaaattgtgttgacgcagtctccagacaccctctctttgtctccaggggaaacagccagtttctcctgcagggccggtcaaattattatcaacagacagttagcctggtaccagcggagacctggccaggctccccggctcctcatctatggcgcgtccaatagggtcactggcatcccagacaggttcagtggcagtgggtctgggacagacttcactctcacgatcaatagactggagcctgaagattttgcagtgtattactgtcagcactatggtggctcaccttggacgttcggccaagggaccaaggtggaaatcaaa

SEQ ID NO: 89 - protein, 215 aa, Homo sapiens
EIVLTQSPDTLSLSPGETASFSCRAGQIIINRQLAWYQRRPGQAPRLLIYGASNRVTGIPDRFSGSGSGTDFTLTINRLEPEDFAVYYCQHYGGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 8 aa, Homo sapiens
GYTFTSHA

SEQ ID NO: 91 - protein, 8 aa, Homo sapiens
INAANGKT

SEQ ID NO: 92 - protein, 11 aa, Homo sapiens
ARRPYGGPFDY

SEQ ID NO: 93 - protein, 5 aa, Homo sapiens
SHAMH

SEQ ID NO: 94 - protein, 17 aa, Homo sapiens
WINAANGKTDYSQNFQG

SEQ ID NO: 95 - protein, 9 aa, Homo sapiens
RPYGGPFDY

SEQ ID NO: 96 - protein, 118 aa, Homo sapiens
QVQFVQSGAEVKKPGASVKVSCKASGYTFTSHAMHWVRQAPGQRLEWMGWINAANGKTDYSQNFQGRVTITRDTYANTVYMELSSLRSEDTAVYYCARRPYGGPFDYWGQGTLVTVSS

SEQ ID NO: 97 - DNA, 354 bp, Homo sapiens
  source 1..354: mol_type = unassigned DNA
caggtccagtttgtgcagtctggggctgaggtgaagaagcctggggcctcagtgaaggtttcctgtaaggcttctggatacaccttcactagccatgctatgcattgggtgcgccaggcccccggacaaaggcttgagtggatgggatggatcaacgctgccaatggtaaaacagattattcacagaacttccagggcagagtcaccattaccagggacacatacgcgaacacagtctacatggaactgagcagcctgagatctgaagacacggctgtgtattactgtgcgagacgcccttacggtggtccctttgactactggggccagggaaccctggtcaccgtctcctca

SEQ ID NO: 98 - protein, 445 aa, Homo sapiens
QVQFVQSGAEVKKPGASVKVSCKASGYTFTSHAMHWVRQAPGQRLEWMGWINAANGKTDYSQNFQGRVTITRDTYANTVYMELSSLRSEDTAVYYCARRPYGGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 6 aa, Homo sapiens
QSISIW

SEQ ID NO: 100 - skipped

SEQ ID NO: 101 - protein, 9 aa, Homo sapiens
QQYNLYPWT

SEQ ID NO: 102 - protein, 11 aa, Homo sapiens
RASQSISIWLA

SEQ ID NO: 103 - protein, 7 aa, Homo sapiens
KASSLES

SEQ ID NO: 104 - protein, 9 aa, Homo sapiens
QQYNLYPWT

SEQ ID NO: 105 - protein, 107 aa, Homo sapiens
DIQMTQSPSTLSASVGDRVTITCRASQSISIWLAWYQQKPGKAPKLLIYKASSLESGVPSRFSGSGSGTEFTLTISSLQPDDFATYYCQQYNLYPWTFGQGTKVEIK

SEQ ID NO: 106 - DNA, 321 bp, Homo sapiens
  source 1..321: mol_type = unassigned DNA
gacatccagatgacccagtctccttccaccctgtctgcatctgtaggagacagagtcaccatcacttgccgggccagtcagagtattagtatctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgtcaacagtataatctttatccgtggacgttcggccaagggaccaaggtggaaatcaaa

SEQ ID NO: 107 - protein, 214 aa, Homo sapiens
DIQMTQSPSTLSASVGDRVTITCRASQSISIWLAWYQQKPGKAPKLLIYKASSLESGVPSRFSGSGSGTEFTLTISSLQPDDFATYYCQQYNLYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 8 aa, Homo sapiens
GFTFSDYY

SEQ ID NO: 109 - protein, 8 aa, Homo sapiens
ISISGSTI

SEQ ID NO: 110 - protein, 10 aa, Homo sapiens
ARRSSGWYDY

SEQ ID NO: 111 - protein, 5 aa, Homo sapiens
DYYMS

SEQ ID NO: 112 - protein, 17 aa, Homo sapiens
YISISGSTIYYADSVKG

SEQ ID NO: 113 - protein, 8 aa, Homo sapiens
RSSGWYDY

SEQ ID NO: 114 - protein, 117 aa, Homo sapiens
QVQLVESGGGLVKPGGSLRLSCAASGFTFSDYYMSWIRQAPGKGLEWVSYISISGSTIYYADSVKGRFTISRDNAMDSLYLQMNSLRAEDTAVYYCARRSSGWYDYWGQGTLVTVSS

SEQ ID NO: 115 - DNA, 351 bp, Homo sapiens
  source 1..351: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtagtaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctca

SEQ ID NO: 116 - protein, 444 aa, Homo sapiens
QVQLVESGGGLVKPGGSLRLSCAASGFTFSDYYMSWIRQAPGKGLEWVSYISISGSTIYYADSVKGRFTISRDNAMDSLYLQMNSLRAEDTAVYYCARRSSGWYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 7 aa, Homo sapiens
QRVVYRY

SEQ ID NO: 118 - skipped

SEQ ID NO: 119 - protein, 9 aa, Homo sapiens
HQYGSSPPT

SEQ ID NO: 120 - protein, 12 aa, Homo sapiens
WASQRVVYRYLA

SEQ ID NO: 121 - protein, 7 aa, Homo sapiens
GAFNRAT

SEQ ID NO: 122 - protein, 9 aa, Homo sapiens
HQYGSSPPT

SEQ ID NO: 123 - protein, 108 aa, Homo sapiens
EIVLTQSPGTLSLSPGERATLSCWASQRVVYRYLAWYQRKPGQAPRLLIYGAFNRATGIPDRFSGSGSGTDFSLTISRLEPEDFAVYYCHQYGSSPPTFGQGTKVEIK

SEQ ID NO: 124 - DNA, 324 bp, Homo sapiens
  source 1..324: mol_type = unassigned DNA
gaaattgtgttgacgcagtctccaggcaccctgtctttgtctccaggggaaagagccaccctctcttgttgggccagtcagagggttgtttacagatacttagcctggtaccagcggaaacctggccaggctcccagacttctcatttatggtgcattcaacagggccactggcatcccagacaggttcagtggcagtgggtctgggacagacttcagtctcactatcagtagactggagcctgaggattttgcagtttattactgtcaccaatatggtagttcaccaccgacgttcggccaagggaccaaggtggaaatcaaa

SEQ ID NO: 125 - protein, 215 aa, Homo sapiens
EIVLTQSPGTLSLSPGERATLSCWASQRVVYRYLAWYQRKPGQAPRLLIYGAFNRATGIPDRFSGSGSGTDFSLTISRLEPEDFAVYYCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 8 aa, Homo sapiens
GFTFITYA

SEQ ID NO: 127 - protein, 8 aa, Homo sapiens
INVGNGNR

SEQ ID NO: 128 - protein, 11 aa, Homo sapiens
ARRPLWGPFDY

SEQ ID NO: 129 - protein, 5 aa, Homo sapiens
TYAFH

SEQ ID NO: 130 - protein, 17 aa, Homo sapiens
WINVGNGNREYSQKFQD

SEQ ID NO: 131 - protein, 9 aa, Homo sapiens
RPLWGPFDY

SEQ ID NO: 132 - protein, 118 aa, Homo sapiens
QVQLVQSGAEVKKPGASVKVSCKATGFTFITYAFHWVRQAPGQRFEWMGWINVGNGNREYSQKFQDRVTITRDTSATTVYMELNSLKSEDTAMYFCARRPLWGPFDYWGQGTLVTVSS

SEQ ID NO: 133 - DNA, 354 bp, Homo sapiens
  source 1..354: mol_type = unassigned DNA
caggtccaacttgtgcagtctggggctgaggtgaagaagcctggggcctcagtgaaggtttcctgcaaggctactggattcaccttcattacctatgctttccattgggtgcgccaggcccccggacaaaggtttgagtggatgggatggatcaacgttggcaatggtaacagagaatattcacagaagttccaggacagagtcaccattaccagggacacatccgcgaccacagtctacatggaactgaacagcctgaaatctgaagacacggctatgtatttctgtgcgagacgccccctctggggtccctttgactactggggccagggaaccctggtcaccgtctcctca

SEQ ID NO: 134 - protein, 445 aa, Homo sapiens
QVQLVQSGAEVKKPGASVKVSCKATGFTFITYAFHWVRQAPGQRFEWMGWINVGNGNREYSQKFQDRVTITRDTSATTVYMELNSLKSEDTAMYFCARRPLWGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 7 aa, Homo sapiens
QIFSNTF

SEQ ID NO: 136 - skipped

SEQ ID NO: 137 - protein, 9 aa, Homo sapiens
QHYGGSPWT

SEQ ID NO: 138 - protein, 12 aa, Homo sapiens
RASQIFSNTFLA

SEQ ID NO: 139 - protein, 7 aa, Homo sapiens
GASKRAT

SEQ ID NO: 140 - protein, 9 aa, Homo sapiens
QHYGGSPWT

SEQ ID NO: 141 - protein, 108 aa, Homo sapiens
EIVLTQSPGTLSLSPGERATLSCRASQIFSNTFLAWYQQKPGQAPRLLVYGASKRATAIPDRFSGSGSGTDFILTINRLEPEDFAVYYCQHYGGSPWTFGRGTKVEIK

SEQ ID NO: 142 - DNA, 324 bp, Homo sapiens
  source 1..324: mol_type = unassigned DNA
gaaattgttttgacacagtctccaggcaccctgtctttgtctccaggggaaagagccaccctctcctgcagggccagtcagatttttagcaacaccttcttagcctggtaccagcagaaacctggccaggctcccaggctcctcgtgtatggtgcatccaagagggccactgccatcccagacaggtttagtggcagtgggtctgggacagacttcattctcaccatcaacagactggagcctgaagattttgcagtatattactgtcaacactatggtgggtcaccgtggacgttcggccgagggaccaaggtggaaatcaaa

SEQ ID NO: 143 - protein, 215 aa, Homo sapiens
EIVLTQSPGTLSLSPGERATLSCRASQIFSNTFLAWYQQKPGQAPRLLVYGASKRATAIPDRFSGSGSGTDFILTINRLEPEDFAVYYCQHYGGSPWTFGRGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 8 aa, Homo sapiens
GYTFTSHA

SEQ ID NO: 145 - protein, 8 aa, Homo sapiens
IHAGNGNS

SEQ ID NO: 146 - protein, 11 aa, Homo sapiens
ARRAIMAPFDL

SEQ ID NO: 147 - protein, 5 aa, Homo sapiens
SHAIH

SEQ ID NO: 148 - protein, 17 aa, Homo sapiens
WIHAGNGNSKQSQNFQD

SEQ ID NO: 149 - protein, 9 aa, Homo sapiens
RAIMAPFDL

SEQ ID NO: 150 - protein, 118 aa, Homo sapiens
QVQLVQSGAEVKKPGASVKVSCKASGYTFTSHAIHWVRQAPGQRLEWMGWIHAGNGNSKQSQNFQDRVTITRDTSASAAYMELSSLRSEDTAVYYCARRAIMAPFDLWGQGTLVTVSS

SEQ ID NO: 151 - DNA, 354 bp, Homo sapiens
  source 1..354: mol_type = unassigned DNA
caggtccagcttgtgcagtctggggctgaggtgaagaagcctggggcctcagtgaaggtttcctgcaaggcttctggatacaccttcactagccatgctatacattgggtgcgccaggcccccggacaacggcttgagtggatgggatggatccacgctggcaatggtaactcaaaacagtcacagaacttccaggacagagtcaccattaccagggacacatccgcgagcgcagcctacatggagctgagcagcctgagatctgaagacacggctgtatattactgtgcgagacgggccataatggccccgtttgacctctggggccagggaaccctggtcaccgtctcctca

SEQ ID NO: 152 - protein, 445 aa, Homo sapiens
QVQLVQSGAEVKKPGASVKVSCKASGYTFTSHAIHWVRQAPGQRLEWMGWIHAGNGNSKQSQNFQDRVTITRDTSASAAYMELSSLRSEDTAVYYCARRAIMAPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - protein, 6 aa, Homo sapiens
QSINNW

SEQ ID NO: 154 - skipped

SEQ ID NO: 155 - protein, 8 aa, Homo sapiens
QQYYSSWT

SEQ ID NO: 156 - protein, 11 aa, Homo sapiens
RASQSINNWLA

SEQ ID NO: 157 - protein, 7 aa, Homo sapiens
KASSLES

SEQ ID NO: 158 - protein, 8 aa, Homo sapiens
QQYYSSWT

SEQ ID NO: 159 - protein, 106 aa, Homo sapiens
DIQMTQSPSTLSASVGDRVTITCRASQSINNWLAWYQQKPGKAPNLLIYKASSLESGVPSRFSGSGSGTEFTLTINSLQPDDFATYYCQQYYSSWTFGQGTKVEIK

SEQ ID NO: 160 - DNA, 318 bp, Homo sapiens
  source 1..318: mol_type = unassigned DNA
gacatccagatgacccagtctccttccaccctgtctgcatctgtaggagacagagtcaccatcacttgccgggccagtcagagtattaataactggttggcctggtatcagcagaaaccagggaaagcccctaacctcctgatctataaggcgtctagtttagaaagtggggtcccatcaaggttcagcggcagtggatctgggacagaattcactctcaccatcaacagcctgcagcctgatgactttgcaacttattactgccaacagtattatagttcttggacgttcggccaagggaccaaggtggaaatcaaa

SEQ ID NO: 161 - protein, 213 aa, Homo sapiens
DIQMTQSPSTLSASVGDRVTITCRASQSINNWLAWYQQKPGKAPNLLIYKASSLESGVPSRFSGSGSGTEFTLTINSLQPDDFATYYCQQYYSSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 8 aa, Homo sapiens
GYTFTSYA

SEQ ID NO: 163 - protein, 8 aa, Homo sapiens
INAGNGNT

SEQ ID NO: 164 - protein, 11 aa, Homo sapiens
ARRGFGEPFDY

SEQ ID NO: 165 - protein, 5 aa, Homo sapiens
SYAVH

SEQ ID NO: 166 - protein, 17 aa, Homo sapiens
WINAGNGNTKFSQKFQG

SEQ ID NO: 167 - protein, 9 aa, Homo sapiens
RGFGEPFDY

SEQ ID NO: 168 - protein, 118 aa, Homo sapiens
QVQLVQSGAEVKKPGASVKVSCKASGYTFTSYAVHWVRQAPGQRLEWMGWINAGNGNTKFSQKFQGRITITRDTSASTTYMELNSLRSEDTAVYYCARRGFGEPFDYWGQGTLVTVSS

SEQ ID NO: 169 - DNA, 354 bp, Homo sapiens
  source 1..354: mol_type = unassigned DNA
caggtccagcttgtgcagtctggggctgaggtgaagaagcctggggcctcagtgaaggtttcctgcaaggcttctggatacaccttcactagctatgctgtccattgggtgcgccaggcccccggacaaaggcttgagtggatgggatggatcaacgctggcaatggtaacacaaaattttcacagaagttccagggcagaatcaccattaccagggacacatccgcgagcacaacctacatggagttgaatagtctgagatctgaagacacggctgtgtattattgtgcgagaagggggttcggggagccatttgactactggggccagggaaccctggtcaccgtctcctca

SEQ ID NO: 170 - protein, 445 aa, Homo sapiens
QVQLVQSGAEVKKPGASVKVSCKASGYTFTSYAVHWVRQAPGQRLEWMGWINAGNGNTKFSQKFQGRITITRDTSASTTYMELNSLRSEDTAVYYCARRGFGEPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 171 - protein, 6 aa, Homo sapiens
QSISNN

SEQ ID NO: 172 - skipped

SEQ ID NO: 173 - protein, 9 aa, Homo sapiens
QQYNIWPFT

SEQ ID NO: 174 - protein, 11 aa, Homo sapiens
RASQSISNNLA

SEQ ID NO: 175 - protein, 7 aa, Homo sapiens
GTSTRAT

SEQ ID NO: 176 - protein, 9 aa, Homo sapiens
QQYNIWPFT

SEQ ID NO: 177 - protein, 107 aa, Homo sapiens
EIVMTQSPATLSVSPGERATLSCRASQSISNNLAWYQQKPGQAPRLLIYGTSTRATGIPARFSGSGSGTEFTLTISTLQSEDFAVYYCQQYNIWPFTFGPGTKVDIK

SEQ ID NO: 178 - DNA, 321 bp, Homo sapiens
  source 1..321: mol_type = unassigned DNA
gaaatagtgatgacgcagtctccagccaccctgtctgtgtctccaggggaaagagccaccctctcctgcagggccagtcagagtattagtaacaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaa

SEQ ID NO: 179 - protein, 214 aa, Homo sapiens
EIVMTQSPATLSVSPGERATLSCRASQSISNNLAWYQQKPGQAPRLLIYGTSTRATGIPARFSGSGSGTEFTLTISTLQSEDFAVYYCQQYNIWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 8 aa, Homo sapiens
GYTFTSYA

SEQ ID NO: 181 - protein, 8 aa, Homo sapiens
INPGNGNT

SEQ ID NO: 182 - protein, 11 aa, Homo sapiens
ARRGFGEPFDY

SEQ ID NO: 183 - protein, 5 aa, Homo sapiens
SYAIH

SEQ ID NO: 184 - protein, 17 aa, Homo sapiens
WINPGNGNTKFSQKFQG

SEQ ID NO: 185 - protein, 9 aa, Homo sapiens
RGFGEPFDY

SEQ ID NO: 186 - protein, 118 aa, Homo sapiens
QVQLVQSGAEVKKPGASVKVSCKASGYTFTSYAIHWVRQAPGQRLEWMGWINPGNGNTKFSQKFQGRITITRDTSASTTYMELNSLRSEDTAVYYCARRGFGEPFDYWGQGTLVTVSS

SEQ ID NO: 187 - DNA, 354 bp, Homo sapiens
  source 1..354: mol_type = unassigned DNA
caggtccagcttgtgcagtctggggctgaggtgaagaagcctggggcctcagtgaaggtttcctgcaaggcttctggatacaccttcactagctatgctatccattgggtgcgccaggcccccggacaaaggcttgagtggatgggatggatcaaccctggcaatggtaacacaaaattttcacagaagttccagggcagaatcaccattaccagggacacatccgcgagcacaacctacatggagctgaacagcctgagatctgaagacacggctgtgtattactgtgcgagaagggggttcggggagccatttgactactggggccagggaaccctggtcaccgtctcctca

SEQ ID NO: 188 - protein, 445 aa, Homo sapiens
QVQLVQSGAEVKKPGASVKVSCKASGYTFTSYAIHWVRQAPGQRLEWMGWINPGNGNTKFSQKFQGRITITRDTSASTTYMELNSLRSEDTAVYYCARRGFGEPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 189 - protein, 6 aa, Homo sapiens
QSISSN

SEQ ID NO: 190 - skipped

SEQ ID NO: 191 - protein, 9 aa, Homo sapiens
QQYNIWPFT

SEQ ID NO: 192 - protein, 11 aa, Homo sapiens
RASQSISSNLA

SEQ ID NO: 193 - protein, 7 aa, Homo sapiens
GTSTRAT

SEQ ID NO: 194 - protein, 9 aa, Homo sapiens
QQYNIWPFT

SEQ ID NO: 195 - protein, 107 aa, Homo sapiens
EIVMTQSPATLSVSPGERATLSCRASQSISSNLAWYQQNPGQAPRLLIYGTSTRATGIPARFSGSGSGTEFTLTISTLQSEDFAVYYCQQYNIWPFTFGPGTKVDIK

SEQ ID NO: 196 - DNA, 322 bp, Homo sapiens
  source 1..322: mol_type = unassigned DNA
gaaatagtgatgacgcagtctccagccaccctgtctgtgtctccaggggaaagagccaccctctcctgcagggccagtcagagtattagcagcaacttagcctggtaccaacagaaccctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaac

SEQ ID NO: 197 - protein, 214 aa, Homo sapiens
EIVMTQSPATLSVSPGERATLSCRASQSISSNLAWYQQNPGQAPRLLIYGTSTRATGIPARFSGSGSGTEFTLTISTLQSEDFAVYYCQQYNIWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 8 aa, Homo sapiens
GYTFTSYA

SEQ ID NO: 199 - protein, 8 aa, Homo sapiens
INAGNGYT

SEQ ID NO: 200 - protein, 13 aa, Homo sapiens
ARDRITIIRPFDY

SEQ ID NO: 201 - protein, 5 aa, Homo sapiens
SYAMH

SEQ ID NO: 202 - protein, 17 aa, Homo sapiens
WINAGNGYTKYSQKFQD

SEQ ID NO: 203 - protein, 11 aa, Homo sapiens
DRITIIRPFDY

SEQ ID NO: 204 - protein, 120 aa, Homo sapiens
QVQLVQSGAEVKKPGASVKVSCKASGYTFTSYAMHWVRQAPGHRLEWLGWINAGNGYTKYSQKFQDRVAITRDTSASTAFMELSSLRSEDTAVYYCARDRITIIRPFDYWGQGTLVTVSS

SEQ ID NO: 205 - DNA, 360 bp, Homo sapiens
  source 1..360: mol_type = unassigned DNA
caggtccaacttgtgcagtctggggctgaggtgaagaagcctggggcctcagtgaaggtttcctgcaaggcttctggatacaccttcactagctatgctatgcattgggtgcgtcaggcccccggacacaggcttgagtggttgggatggatcaacgctggcaatggttacacaaaatattcacagaaattccaggacagagtcgccattaccagggacacatccgcgagcacagccttcatggagctgagcagcctgagatctgaagacacggctgtgtattactgtgcgagagatcgtattactattattcggccctttgactactggggccagggaaccctggtcaccgtctcctca

SEQ ID NO: 206 - protein, 447 aa, Homo sapiens
QVQLVQSGAEVKKPGASVKVSCKASGYTFTSYAMHWVRQAPGHRLEWLGWINAGNGYTKYSQKFQDRVAITRDTSASTAFMELSSLRSEDTAVYYCARDRITIIRPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 207 - protein, 7 aa, Homo sapiens
QSISSSY

SEQ ID NO: 208 - skipped

SEQ ID NO: 209 - protein, 8 aa, Homo sapiens
QLYGSPFT

SEQ ID NO: 210 - protein, 12 aa, Homo sapiens
RASQSISSSYLA

SEQ ID NO: 211 - protein, 7 aa, Homo sapiens
GASSRAT

SEQ ID NO: 212 - protein, 8 aa, Homo sapiens
QLYGSPFT

SEQ ID NO: 213 - protein, 107 aa, Homo sapiens
EIVLTQSPGTLSLSPGERATLSCRASQSISSSYLAWFQQKPGQAPRLLIYGASSRATGIPDRFSGNGSGTDFTLTISRLEPEDFAVYYCQLYGSPFTFGPGTKMDIK

SEQ ID NO: 214 - DNA, 322 bp, Homo sapiens
  source 1..322: mol_type = unassigned DNA
gaaattgtgttgacgcagtctccaggcaccctgtctttgtctccaggggaaagagccaccctctcctgcagggccagtcagagtattagcagcagctacttagcctggttccagcaaaaacctggccaggctcccaggctcctcatctatggtgcatccagcagggccactggcatcccagacaggttcagtggcaatgggtctgggacagacttcactctcaccatcagcagactggagcctgaagattttgcagtgtattactgtcagctgtatggtagcccattcactttcggccctgggaccaaaatggatattaaac

SEQ ID NO: 215 - protein, 214 aa, Homo sapiens
EIVLTQSPGTLSLSPGERATLSCRASQSISSSYLAWFQQKPGQAPRLLIYGASSRATGIPDRFSGNGSGTDFTLTISRLEPEDFAVYYCQLYGSPFTFGPGTKMDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 8 aa, Homo sapiens
GYTFTSYA

SEQ ID NO: 217 - protein, 8 aa, Homo sapiens
INVGNGKT

SEQ ID NO: 218 - protein, 11 aa, Homo sapiens
ARRGFGEPFDY

SEQ ID NO: 219 - protein, 5 aa, Homo sapiens
SYAIH

SEQ ID NO: 220 - protein, 17 aa, Homo sapiens
WINVGNGKTKFSQKLQG

SEQ ID NO: 221 - protein, 9 aa, Homo sapiens
RGFGEPFDY

SEQ ID NO: 222 - protein, 118 aa, Homo sapiens
QVQLVQSGAEVKKPGASVKVSCKASGYTFTSYAIHWVRQAPGQRLEWMGWINVGNGKTKFSQKLQGRITITRDTSASTTYMELNSLRSEDTAVFYCARRGFGEPFDYWGQGTLVTVSS

SEQ ID NO: 223 - DNA, 354 bp, Homo sapiens
  source 1..354: mol_type = unassigned DNA
caggtccagcttgtgcagtctggggctgaggtgaagaagcctggggcctcagtgaaagtttcctgcaaggcttctggatacaccttcactagctatgctatccattgggtgcgccaggcccccggacaaaggcttgagtggatgggatggatcaacgttggcaatggtaaaacaaaattttcacagaagttacagggcagaatcaccattaccagggacacatccgcgagcacaacctacatggagctgaacagcctgagatctgaagacacggctgtgttttactgtgcgagaagggggttcggggagccatttgactactggggccagggaaccctggtcaccgtctcctca

SEQ ID NO: 224 - protein, 445 aa, Homo sapiens
QVQLVQSGAEVKKPGASVKVSCKASGYTFTSYAIHWVRQAPGQRLEWMGWINVGNGKTKFSQKLQGRITITRDTSASTTYMELNSLRSEDTAVFYCARRGFGEPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - protein, 6 aa, Homo sapiens
QSISSN

SEQ ID NO: 226 - skipped

SEQ ID NO: 227 - protein, 9 aa, Homo sapiens
QQYNIWPFT

SEQ ID NO: 228 - protein, 11 aa, Homo sapiens
RASQSISSNLA

SEQ ID NO: 229 - protein, 7 aa, Homo sapiens
GTSTRAT

SEQ ID NO: 230 - protein, 9 aa, Homo sapiens
QQYNIWPFT

SEQ ID NO: 231 - protein, 107 aa, Homo sapiens
EIVMTQSPATLSVSPGERATLSCRASQSISSNLAWYQQKPGQAPRLLIYGTSTRATGIPARFSGSGSGTEFTLTISTLQSEDFAVYYCQQYNIWPFTFGPGTKVDIK

SEQ ID NO: 232 - DNA, 321 bp, Homo sapiens
  source 1..321: mol_type = unassigned DNA
gaaatagtgatgacgcagtctccagccaccctgtctgtgtctccaggggaaagagccaccctctcctgcagggccagtcagagtattagtagcaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaa

SEQ ID NO: 233 - protein, 214 aa, Homo sapiens
EIVMTQSPATLSVSPGERATLSCRASQSISSNLAWYQQKPGQAPRLLIYGTSTRATGIPARFSGSGSGTEFTLTISTLQSEDFAVYYCQQYNIWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 8 aa, Homo sapiens
GYTFTSYA

SEQ ID NO: 235 - protein, 8 aa, Homo sapiens
INAGNGNT

SEQ ID NO: 236 - protein, 11 aa, Homo sapiens
ARRGFGEPFDY

SEQ ID NO: 237 - protein, 5 aa, Homo sapiens
SYAIH

SEQ ID NO: 238 - protein, 17 aa, Homo sapiens
WINAGNGNTKYSQKFQG

SEQ ID NO: 239 - protein, 9 aa, Homo sapiens
RGFGEPFDY

SEQ ID NO: 240 - protein, 118 aa, Homo sapiens
QVQLVQSGAEVKKPGASVKVSCKASGYTFTSYAIHWVRQAPGQRLEWMGWINAGNGNTKYSQKFQGRITITRDTSASTAYMELSSLRSEDTAVYYCARRGFGEPFDYWGQGTLVTVSS

SEQ ID NO: 241 - DNA, 354 bp, Homo sapiens
  source 1..354: mol_type = unassigned DNA
caggtccagcttgtgcagtctggggctgaggtgaagaagcctggggcctcagtgaaggtttcctgcaaggcttctggatacaccttcactagctatgctatccattgggtgcgccaggcccccggacaaaggcttgagtggatgggatggatcaacgctggcaatggtaacacaaaatattcacagaagttccagggcagaatcaccattaccagggacacatccgcgagcacagcctacatggagctgagcagcctgagatctgaagacacggctgtgtattactgtgcgagaagggggttcggggagccatttgactactggggccagggaaccctggtcaccgtctcctca

SEQ ID NO: 242 - protein, 445 aa, Homo sapiens
QVQLVQSGAEVKKPGASVKVSCKASGYTFTSYAIHWVRQAPGQRLEWMGWINAGNGNTKYSQKFQGRITITRDTSASTAYMELSSLRSEDTAVYYCARRGFGEPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 243 - protein, 6 aa, Homo sapiens
QSISSN

SEQ ID NO: 244 - skipped

SEQ ID NO: 245 - protein, 9 aa, Homo sapiens
QQYNIWPFT

SEQ ID NO: 246 - protein, 11 aa, Homo sapiens
RASQSISSNLA

SEQ ID NO: 247 - protein, 7 aa, Homo sapiens
GTSTRAT

SEQ ID NO: 248 - protein, 9 aa, Homo sapiens
QQYNIWPFT

SEQ ID NO: 249 - protein, 107 aa, Homo sapiens; identical to SEQ ID NO: 231

SEQ ID NO: 250 - DNA, 321 bp, Homo sapiens
  source 1..321: mol_type = unassigned DNA
gaaatagtgatgacgcagtctccagccaccctgtctgtgtctccaggggaaagagccaccctctcctgcagggccagtcagagtattagcagcaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaa

SEQ ID NO: 251 - protein, 214 aa, Homo sapiens; identical to SEQ ID NO: 233

SEQ ID NO: 252 - protein, 8 aa, Homo sapiens
GYTFTTYA

SEQ ID NO: 253 - protein, 8 aa, Homo sapiens
INAGNGRT

SEQ ID NO: 254 - protein, 11 aa, Homo sapiens
ARRGFGEPFDQ

SEQ ID NO: 255 - protein, 5 aa, Homo sapiens
TYAIH

SEQ ID NO: 256 - protein, 17 aa, Homo sapiens
WINAGNGRTEYSEKFQG

SEQ ID NO: 257 - protein, 9 aa, Homo sapiens
RGFGEPFDQ

SEQ ID NO: 258 - protein, 118 aa, Homo sapiens
QVHLVQSGAEVKKPGASVKVSCKTSGYTFTTYAIHWVRQAPGQGLEWMGWINAGNGRTEYSEKFQGRVTITRDTSASTVYMDLSSLRSGDTAVYYCARRGFGEPFDQWGQGTLVTVSS

SEQ ID NO: 259 - DNA, 354 bp, Homo sapiens
  source 1..354: mol_type = unassigned DNA
caggtccaccttgtgcagtctggggctgaggtgaagaagcctggggcctcagtgaaggtgtcctgcaagacttctggatacaccttcaccacctatgctattcattgggtgcgccaggcccccggacaagggcttgagtggatgggatggatcaacgctggcaatggtagaacagaatattcagagaagtttcagggcagagtcaccattaccagggacacttccgcgagtacagtctacatggacctgagcagcctgagatctggagacacggctgtgtattactgtgcgagaaggggattcggggagccatttgaccaatggggccagggaaccctggtcaccgtctcctca

SEQ ID NO: 260 - protein, 445 aa, Homo sapiens
QVHLVQSGAEVKKPGASVKVSCKTSGYTFTTYAIHWVRQAPGQGLEWMGWINAGNGRTEYSEKFQGRVTITRDTSASTVYMDLSSLRSGDTAVYYCARRGFGEPFDQWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 261 - protein, 6 aa, Homo sapiens
QSVSSN

SEQ ID NO: 262 - skipped

SEQ ID NO: 263 - protein, 9 aa, Homo sapiens
QQYNNWPFI

SEQ ID NO: 264 - protein, 11 aa, Homo sapiens
RASQSVSSNLA

SEQ ID NO: 265 - protein, 7 aa, Homo sapiens
GASTRAT

SEQ ID NO: 266 - protein, 9 aa, Homo sapiens
QQYNNWPFI

SEQ ID NO: 267 - protein, 107 aa, Homo sapiens
EIVMTQSPATLSVSPGERATLSCRASQSVSSNLAWYQHQPGQAPRLLIYGASTRATGFPPRFSGSGSGTDFTLTINSLQSEDFAVYYCQQYNNWPFIFGPGTKLDIT

SEQ ID NO: 268 - DNA, 321 bp, Homo sapiens
  source 1..321: mol_type = unassigned DNA
gaaatagtgatgacgcagtctccagccaccctgtctgtgtctccaggggaaagagccaccctctcctgcagggccagtcagagtgttagcagcaacttagcctggtaccagcaccaacctggccaggctcccaggctcctcatctatggtgcatccaccagggccactggtttcccacccaggttcagtggcagtgggtctgggacagacttcactctcaccatcaacagcctgcagtctgaagattttgcagtttattactgtcagcagtataataactggcctttcattttcggccctgggaccaaactggatatcaca

SEQ ID NO: 269 - protein, 214 aa, Homo sapiens
EIVMTQSPATLSVSPGERATLSCRASQSVSSNLAWYQHQPGQAPRLLIYGASTRATGFPPRFSGSGSGTDFTLTINSLQSEDFAVYYCQQYNNWPFIFGPGTKLDITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 8 aa, Homo sapiens
GFTFSDYY

SEQ ID NO: 271 - protein, 8 aa, Homo sapiens
ISISGSTI

SEQ ID NO: 272 - protein, 10 aa, Homo sapiens
ARRSSGWYDY

SEQ ID NO: 273 - protein, 5 aa, Homo sapiens
DYYMS

SEQ ID NO: 274 - protein, 17 aa, Homo sapiens
YISISGSTIYYADSVKG

SEQ ID NO: 275 - protein, 8 aa, Homo sapiens
RSSGWYDY

SEQ ID NO: 276 - protein, 117 aa, Homo sapiens
QVQLVESGGGLVKPGGSLRLSCAASGFTFSDYYMSWIRQAPGKGLEWVSYISISGSTIYYADSVKGRFTISRDNAKNSLFLQMNSLRAEDTAVYYCARRSSGWYDYWGQGTLVTVSS

SEQ ID NO: 277 - DNA, 351 bp, Homo sapiens
  source 1..351: mol_type = unassigned DNA
caggtgcaactggtggagtctgggggaggcttggtcaagcctggagggtccctgagactctcctgtgcagcctctggattcaccttcagtgactactacatgagctggatccgccaggctccagggaaggggctggagtgggtttcatacattagtattagtggtagtaccatatactacgcagactctgtgaagggccgattcaccatctccagggacaacgccaagaactcactgtttctgcaaatgaacagcctgagagccgaggacacggccgtgtattactgtgcgagaagaagcagtggctggtacgactactggggccagggaaccctggtcaccgtctcctca

SEQ ID NO: 278 - protein, 444 aa, Homo sapiens
QVQLVESGGGLVKPGGSLRLSCAASGFTFSDYYMSWIRQAPGKGLEWVSYISISGSTIYYADSVKGRFTISRDNAKNSLFLQMNSLRAEDTAVYYCARRSSGWYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 279 - protein, 7 aa, Homo sapiens
QSVSSNY

SEQ ID NO: 280 - skipped

SEQ ID NO: 281 - protein, 9 aa, Homo sapiens
HQYGSSPPT

SEQ ID NO: 282 - protein, 12 aa, Homo sapiens
RASQSVSSNYLA

SEQ ID NO: 283 - protein, 7 aa, Homo sapiens
GASIRAT

SEQ ID NO: 284 - protein, 9 aa, Homo sapiens
HQYGSSPPT

SEQ ID NO: 285 - protein, 108 aa, Homo sapiens
EIVLTQSPGTLSLSPGERATLSCRASQSVSSNYLAWYQQKPGQAPRLLIYGASIRATGIPDRFSGSGSGTDFTLTISRLEPEDFAVYSCHQYGSSPPTFGQGTKVEIK

SEQ ID NO: 286 - DNA, 325 bp, Homo sapiens
  source 1..325: mol_type = unassigned DNA
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccatcagggccactggcatcccagacaggttcagtggcagtgggtctgggacagacttcactctcaccatcagcagactggagcctgaagattttgcagtgtattcctgtcaccagtatggtagctcacctccgacgttcggccaagggaccaaggtggaaatcaaac

SEQ ID NO: 287 - protein, 215 aa, Homo sapiens
EIVLTQSPGTLSLSPGERATLSCRASQSVSSNYLAWYQQKPGQAPRLLIYGASIRATGIPDRFSGSGSGTDFTLTISRLEPEDFAVYSCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 8 aa, Homo sapiens
GFTFSDYY

SEQ ID NO: 289 - protein, 8 aa, Homo sapiens
ISISGTTI

SEQ ID NO: 290 - protein, 10 aa, Homo sapiens
ARRSSGWYDY

SEQ ID NO: 291 - protein, 5 aa, Homo sapiens
DYYMS

SEQ ID NO: 292 - protein, 17 aa, Homo sapiens
YISISGTTIYYADSVKG

SEQ ID NO: 293 - protein, 8 aa, Homo sapiens
RSSGWYDY

SEQ ID NO: 294 - protein, 117 aa, Homo sapiens
QVQLVESGGGLVKPGGSLRLSCAASGFTFSDYYMSWIRQAPGKGLEWVSYISISGTTIYYADSVKGRFTISRDNAMDSLYLQMNSLRAEDTAVYYCARRSSGWYDYWGQGTLVTVSS

SEQ ID NO: 295 - DNA, 351 bp, Homo sapiens
  source 1..351: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtactaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctca

SEQ ID NO: 296 - protein, 444 aa, Homo sapiens
QVQLVESGGGLVKPGGSLRLSCAASGFTFSDYYMSWIRQAPGKGLEWVSYISISGTTIYYADSVKGRFTISRDNAMDSLYLQMNSLRAEDTAVYYCARRSSGWYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 297 - protein, 7 aa, Homo sapiens
QRVVYRY

SEQ ID NO: 298 - skipped

SEQ ID NO: 299 - protein, 9 aa, Homo sapiens
HQYGSSPPT

SEQ ID NO: 300 - protein, 12 aa, Homo sapiens
WASQRVVYRYLA

SEQ ID NO: 301 - protein, 7 aa, Homo sapiens
GAFNRAT

SEQ ID NO: 302 - protein, 9 aa, Homo sapiens
HQYGSSPPT

SEQ ID NO: 303 - protein, 108 aa, Homo sapiens; identical to SEQ ID NO: 123

SEQ ID NO: 304 - DNA, 324 bp, Homo sapiens; identical to SEQ ID NO: 124
  source 1..324: mol_type = unassigned DNA

SEQ ID NO: 305 - protein, 215 aa, Homo sapiens; identical to SEQ ID NO: 125

SEQ ID NO: 306 - protein, 8 aa, Homo sapiens
GFTFSDYY

SEQ ID NO: 307 - protein, 8 aa, Homo sapiens
ISISGSTI

SEQ ID NO: 308 - protein, 10 aa, Homo sapiens
ARRSSGWYDY

SEQ ID NO: 309 - protein, 5 aa, Homo sapiens
DYYMS

SEQ ID NO: 310 - protein, 17 aa, Homo sapiens
YISISGSTIYYADSVKG

SEQ ID NO: 311 - protein, 8 aa, Homo sapiens
RSSGWYDY

SEQ ID NO: 312 - protein, 117 aa, Homo sapiens
QVQLVESGGGLVKPGGSLRLSCAASGFTFSDYYMSWIRQAPGKGLEWVSYISISGSTIYYADSVKGRFTISRDNAKNSLYLQMNSLRAEDTAVYYCARRSSGWYDYWGQGTLVTVSS

SEQ ID NO: 313 - DNA, 351 bp, Homo sapiens
  source 1..351: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtattagtggtagtaccatatactacgcagactctgtgaagggccgattcaccatctccagggacaacgccaagaactcactgtatctgcaaatgaacagcctgagagccgaggacacggccgtgtattactgtgcgagaagaagcagtggctggtacgactactggggccagggaaccctggtcaccgtctcctca

SEQ ID NO: 314 - protein, 444 aa, Homo sapiens
QVQLVESGGGLVKPGGSLRLSCAASGFTFSDYYMSWIRQAPGKGLEWVSYISISGSTIYYADSVKGRFTISRDNAKNSLYLQMNSLRAEDTAVYYCARRSSGWYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 315 - protein, 7 aa, Homo sapiens
QSVSSNY

SEQ ID NO: 316 - skipped

SEQ ID NO: 317 - protein, 9 aa, Homo sapiens
HQYGSSPPT

SEQ ID NO: 318 - protein, 12 aa, Homo sapiens
RASQSVSSNYLA

SEQ ID NO: 319 - protein, 7 aa, Homo sapiens
GASIRAT

SEQ ID NO: 320 - protein, 9 aa, Homo sapiens
HQYGSSPPT

SEQ ID NO: 321 - protein, 108 aa, Homo sapiens
EIVLTQSPGTLSLSPGERATLSCRASQSVSSNYLAWYQQKPGQAPRLLIYGASIRATGIPDRFSGSGSGTDFTLTISRLEPEDFAVYCCHQYGSSPPTFGQGTKVEIK

SEQ ID NO: 322 - DNA, 324 bp, Homo sapiens
  source 1..324: mol_type = unassigned DNA
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccatcagggccactggcatcccagacaggttcagtggcagtgggtctgggacagacttcactctcaccatcagcagactggagcctgaagattttgcagtgtattgctgtcaccagtatggtagctcacctccgacgttcggccaagggaccaaggtggaaatcaaa

SEQ ID NO: 323 - protein, 215 aa, Homo sapiens
EIVLTQSPGTLSLSPGERATLSCRASQSVSSNYLAWYQQKPGQAPRLLIYGASIRATGIPDRFSGSGSGTDFTLTISRLEPEDFAVYCCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 8 aa, Homo sapiens
GFTFSDYF

SEQ ID NO: 325 - protein, 8 aa, Homo sapiens
ISISGSTI

SEQ ID NO: 326 - protein, 10 aa, Homo sapiens
ARRTSGWYDF

SEQ ID NO: 327 - protein, 5 aa, Homo sapiens
DYFMS

SEQ ID NO: 328 - protein, 17 aa, Homo sapiens
YISISGSTIYYADSVKG

SEQ ID NO: 329 - protein, 8 aa, Homo sapiens
RTSGWYDF

SEQ ID NO: 330 - protein, 117 aa, Homo sapiens
QVQLVESGGGLVKPGGSLRLSCAASGFTFSDYFMSWIRQAPGKGLEWVSYISISGSTIYYADSVKGRFTISRDNARNSLFLQMNSLRAEDTAIYYCARRTSGWYDFWGQGTLVTVSS

SEQ ID NO: 331 - DNA, 351 bp, Homo sapiens
  source 1..351: mol_type = unassigned DNA
caggtgcagctggtggagtctgggggaggcttggtcaagcctggagggtccctgcgactctcctgtgcagcctctggattcaccttcagtgactacttcatgagctggatccgccaggctccagggaaggggctggagtgggtttcgtacattagtattagtggtagtaccatatactacgcagactctgtgaagggccgattcaccatctccagggacaacgccaggaactcactgtttctgcaaatgaacagcctgagagccgaggacacggccatctattactgtgcgagaagaaccagtggctggtacgacttctggggccagggaaccctggtcaccgtctcctca

SEQ ID NO: 332 - protein, 444 aa, Homo sapiens
QVQLVESGGGLVKPGGSLRLSCAASGFTFSDYFMSWIRQAPGKGLEWVSYISISGSTIYYADSVKGRFTISRDNARNSLFLQMNSLRAEDTAIYYCARRTSGWYDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 333 - protein, 7 aa, Homo sapiens
QSVSYSY

SEQ ID NO: 334 - skipped

SEQ ID NO: 335 - protein, 9 aa, Homo sapiens
HQYGSSPPT

SEQ ID NO: 336 - protein, 12 aa, Homo sapiens
RASQSVSYSYLA

SEQ ID NO: 337 - protein, 7 aa, Homo sapiens
GASSRAT

SEQ ID NO: 338 - protein, 9 aa, Homo sapiens
HQYGSSPPT

SEQ ID NO: 339 - protein, 108 aa, Homo sapiens
EIVLTQSPGTLSLSPGERATLSCRASQSVSYSYLAWYQQKPGQAPRLLIYGASSRATGIPDRFSGSGSGTDFTLTISRLEPEDFAVYCCHQYGSSPPTFGQGTKVEIK

SEQ ID NO: 340 - DNA, 324 bp, Homo sapiens
  source 1..324: mol_type = unassigned DNA
gaaattgtgttgacgcagtctccaggcaccctgtctttgtctccaggggaaagagccaccctctcctgcagggccagtcagagtgttagctacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgttgtcaccagtatggtagttcacctccgacgttcggccaagggaccaaggtggaaatcaaa

SEQ ID NO: 341 - protein, 215 aa, Homo sapiens
EIVLTQSPGTLSLSPGERATLSCRASQSVSYSYLAWYQQKPGQAPRLLIYGASSRATGIPDRFSGSGSGTDFTLTISRLEPEDFAVYCCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 8 aa, Homo sapiens
GFTFSDFY

SEQ ID NO: 343 - protein, 8 aa, Homo sapiens
ISISGTTI

SEQ ID NO: 344 - protein, 10 aa, Homo sapiens
ARRTSGWYDF

SEQ ID NO: 345 - protein, 5 aa, Homo sapiens
DFYMS

SEQ ID NO: 346 - protein, 17 aa, Homo sapiens
YISISGTTIYYADSVKG

SEQ ID NO: 347 - protein, 8 aa, Homo sapiens
RTSGWYDF

SEQ ID NO: 348 - protein, 117 aa, Homo sapiens
QVQLVESGGGLVKPGGSLRLSCAASGFTFSDFYMSWIRQAPGRGLEWVSYISISGTTIYYADSVKGRFTISRDNARNSLYLQMNSLRAEDTAVYYCARRTSGWYDFWGQGTLVTVSS

SEQ ID NO: 349 - DNA, 351 bp, Homo sapiens
  source 1..351: mol_type = unassigned DNA
caggtgcagctggtggagtctgggggaggcttggtcaagcctggagggtcccttagactctcctgtgcagcctctggattcaccttcagtgacttctacatgagctggatccgccaggctccagggagggggctggagtgggtttcgtacattagtattagtggtactaccatatactacgcagactctgtgaagggccgattcaccatctccagggacaacgccaggaactcactgtatctgcaaatgaacagcctgagagccgaggacacggccgtgtattactgtgcgagaagaaccagtggctggtacgacttctggggccagggaaccctggtcaccgtctcctca

SEQ ID NO: 350 - protein, 444 aa, Homo sapiens
QVQLVESGGGLVKPGGSLRLSCAASGFTFSDFYMSWIRQAPGRGLEWVSYISISGTTIYYADSVKGRFTISRDNARNSLYLQMNSLRAEDTAVYYCARRTSGWYDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 351 - protein, 7 aa, Homo sapiens
QSVSYSY

SEQ ID NO: 352 - skipped

SEQ ID NO: 353 - protein, 9 aa, Homo sapiens
HQYGSSPPT

SEQ ID NO: 354 - protein, 12 aa, Homo sapiens
RASQSVSYSYLA

SEQ ID NO: 355 - protein, 7 aa, Homo sapiens
GASSRAT

SEQ ID NO: 356 - protein, 9 aa, Homo sapiens
HQYGSSPPT

SEQ ID NO: 357 - protein, 108 aa, Homo sapiens
EIVLTQSPGTLSLSPGERATLSCRASQSVSYSYLAWYQQKPGQAPRLLIYGASSRATGIPDRFSGSGSGTDFTLTISRLEPEDFAVYCCHQYGSSPPTFGQGTKVEMK

SEQ ID NO: 358 - DNA, 324 bp, Homo sapiens
  source 1..324: mol_type = unassigned DNA
gaaattgtgttgacgcagtctccaggcaccctgtctttgtctccaggggaaagagccaccctctcctgcagggccagtcagagtgttagctacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgctgtcaccagtatggtagttcacctccgacgttcggccaagggaccaaggtggaaatgaaa

SEQ ID NO: 359 - protein, 215 aa, Homo sapiens
EIVLTQSPGTLSLSPGERATLSCRASQSVSYSYLAWYQQKPGQAPRLLIYGASSRATGIPDRFSGSGSGTDFTLTISRLEPEDFAVYCCHQYGSSPPTFGQGTKVEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 8 aa, Homo sapiens
GFTFSDYY

SEQ ID NO: 361 - protein, 8 aa, Homo sapiens
ISISGSTI

SEQ ID NO: 362 - protein, 10 aa, Homo sapiens
ARRTSGWYDF

SEQ ID NO: 363 - protein, 5 aa, Homo sapiens
DYYMS

SEQ ID NO: 364 - protein, 17 aa, Homo sapiens
YISISGSTIYYADSVKG

SEQ ID NO: 365 - protein, 8 aa, Homo sapiens
RTSGWYDF

SEQ ID NO: 366 - protein, 117 aa, Homo sapiens
QVQLVESGGGLVRPGGSLRLSCAASGFTFSDYYMSWIRQAPGKGLEWVSYISISGSTIYYADSVKGRFTISRDNARDSLYLQMNSLRAEDTAVYYCARRTSGWYDFWGQGTLVTVSS

SEQ ID NO: 367 - DNA, 351 bp, Homo sapiens
  source 1..351: mol_type = unassigned DNA
caggtgcagctggtggagtctgggggaggcttggtcaggcctggagggtccctgagactctcctgtgcagcctctggattcaccttcagtgactactacatgagctggatccgccaggctccagggaaggggctggagtgggtttcgtacattagtattagtggtagtaccatatactacgcagactctgtgaagggccgattcaccatctccagggacaacgccagggactcactttatctgcaaatgaacagcctgagagccgaggacacggccgtgtattactgtgcgagaagaaccagtggctggtacgacttctggggccagggaaccctggtcaccgtctcctca

SEQ ID NO: 368 - protein, 444 aa, Homo sapiens
QVQLVESGGGLVRPGGSLRLSCAASGFTFSDYYMSWIRQAPGKGLEWVSYISISGSTIYYADSVKGRFTISRDNARDSLYLQMNSLRAEDTAVYYCARRTSGWYDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 369 - protein, 7 aa, Homo sapiens
QSVSYRY

SEQ ID NO: 370 - skipped

SEQ ID NO: 371 - protein, 9 aa, Homo sapiens
HQYGSSPPT

SEQ ID NO: 372 - protein, 12 aa, Homo sapiens
RASQSVSYRYLA

SEQ ID NO: 373 - protein, 7 aa, Homo sapiens
GASSRAT

SEQ ID NO: 374 - protein, 9 aa, Homo sapiens
HQYGSSPPT

SEQ ID NO: 375 - protein, 108 aa, Homo sapiens
EIVLTQSPGTLSLSPGERATLSCRASQSVSYRYLAWYQQKPGQAPRLLIYGASSRATGIPDRFSGSGSGTDFTLTISRLEPEDFAVYCCHQYGSSPPTFGQGTKVEIK

SEQ ID NO: 376 - DNA, 324 bp, Homo sapiens
  source 1..324: mol_type = unassigned DNA
gaaattgtgttgacgcagtctccaggcaccctgtctttgtctccaggggaaagagccaccctctcctgtagggccagtcagagtgttagctacaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgctgtcaccagtatggtagttcacctccgacgttcggccaagggaccaaggtggaaatcaaa

SEQ ID NO: 377 - protein, 215 aa, Homo sapiens
EIVLTQSPGTLSLSPGERATLSCRASQSVSYRYLAWYQQKPGQAPRLLIYGASSRATGIPDRFSGSGSGTDFTLTISRLEPEDFAVYCCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 378 - protein, 8 aa, Homo sapiens
GFTFSDYY

SEQ ID NO: 379 - protein, 8 aa, Homo sapiens
ISISGITI

SEQ ID NO: 380 - protein, 10 aa, Homo sapiens
ARRSSGWYDY

SEQ ID NO: 381 - protein, 5 aa, Homo sapiens
DYYMS

SEQ ID NO: 382 - protein, 17 aa, Homo sapiens
YISISGITIYYADSVKG

SEQ ID NO: 383 - protein, 8 aa, Homo sapiens
RSSGWYDY

SEQ ID NO: 384 - protein, 117 aa, Homo sapiens
QVQLVESGGGLVKPGGSLRLSCAASGFTFSDYYMSWIRQAPGKGLEWVSYISISGITIYYADSVKGRFTISRDNARNSLYLQMNSLRAEDTAVYYCARRSSGWYDYWGQGTLVTVSS

SEQ ID NO: 385 - DNA, 351 bp, Homo sapiens
  source 1..351: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggcatcaccatctactacgccgactccgtgaagggcagattcaccatctccagagacaacgcccggaactccctgtacctgcagatgaactctctgagagccgaggacaccgccgtgtactactgcgcccgtagatcctctggatggtacgactattggggccagggcaccctggtcacagtttctagt

SEQ ID NO: 386 - protein, 444 aa, Homo sapiens
QVQLVESGGGLVKPGGSLRLSCAASGFTFSDYYMSWIRQAPGKGLEWVSYISISGITIYYADSVKGRFTISRDNARNSLYLQMNSLRAEDTAVYYCARRSSGWYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 387 - protein, 7 aa, Homo sapiens
QSVSYNY

SEQ ID NO: 388 - skipped

SEQ ID NO: 389 - protein, 9 aa, Homo sapiens
HQYGSSPPT

SEQ ID NO: 390 - protein, 12 aa, Homo sapiens
RASQSVSYNYLA

SEQ ID NO: 391 - protein, 7 aa, Homo sapiens
GASIRAT

SEQ ID NO: 392 - protein, 9 aa, Homo sapiens
HQYGSSPPT

SEQ ID NO: 393 - protein, 108 aa, Homo sapiens
EIVLTQSPGTLSLSPGERATLSCRASQSVSYNYLAWYQQKPGQAPRLLIYGASIRATGIPDRFSGSGSGTDFTLTISRLEPEDFAVYCCHQYGSSPPTFGQGTKVEIK

SEQ ID NO: 394 - DNA, 324 bp, Homo sapiens
  source 1..324: mol_type = unassigned DNA
gaaattgtgctgactcagtcccctggcacactgtctttgagccctggcgagagagctaccctgtcctgtagagcctctcagtccgtgtcctacaactacctggcctggtatcagcagaagcccggccaggctcctagactgttgatctacggcgcctccatcagagccacaggcatccctgatagattctccggctctggctctggcaccgacttcaccctgaccatctccagactggaacccgaggacttcgccgtgtactgctgtcaccagtacggctctagccctcctacctttggacagggcaccaaggtggaaatcaaa

SEQ ID NO: 395 - protein, 215 aa, Homo sapiens
EIVLTQSPGTLSLSPGERATLSCRASQSVSYNYLAWYQQKPGQAPRLLIYGASIRATGIPDRFSGSGSGTDFTLTISRLEPEDFAVYCCHQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 396 - protein, 8 aa, Homo sapiens
GYTFTSYA

SEQ ID NO: 397 - protein, 8 aa, Homo sapiens
INPYNRGT

SEQ ID NO: 398 - protein, 11 aa, Homo sapiens
ARRPFGNAMDI

SEQ ID NO: 399 - protein, 5 aa, Homo sapiens
SYAMH

SEQ ID NO: 400 - protein, 17 aa, Homo sapiens
YINPYNRGTKYNENFKG

SEQ ID NO: 401 - protein, 9 aa, Homo sapiens
RPFGNAMDI

SEQ ID NO: 402 - protein, 118 aa, Homo sapiens
QVQLVQSGAEVKKPGSSVKVSCKASGYTFTSYAMHWVRQAPGQGLEWMGYINPYNDGTKYNENFKGRVTITADESTSTAYMELSSLRSEDTAVYYCARRPFGNAMDIWGQGTLVTVSS

SEQ ID NO: 403 - protein, 445 aa, Homo sapiens
QVQLVQSGAEVKKPGSSVKVSCKASGYTFTSYAMHWVRQAPGQGLEWMGYINPYNDGTKYNENFKGRVTITADESTSTAYMELSSLRSEDTAVYYCARRPFGNAMDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 404 - protein, 6 aa, Homo sapiens
ENIYRN

SEQ ID NO: 405 - skipped

SEQ ID NO: 406 - protein, 9 aa, Homo sapiens
QGIWGTPLT

SEQ ID NO: 407 - protein, 11 aa, Homo sapiens
RSSENIYRNLA

SEQ ID NO: 408 - protein, 7 aa, Homo sapiens
AATNLAD

SEQ ID NO: 409 - protein, 9 aa, Homo sapiens
QGIWGTPLT

SEQ ID NO: 410 - protein, 107 aa, Homo sapiens
DIQMTQSPSSLSASVGDRVTITCRSSENIYRNLAWYQQKPGKAPKLLIYAATNLADGVPSRFSGSGSGTDFTLTISSLQPEDFATYYCQGIWGTPLTFGGGTKVEIK

SEQ ID NO: 411 - protein, 214 aa, Homo sapiens
DIQMTQSPSSLSASVGDRVTITCRSSENIYRNLAWYQQKPGKAPKLLIYAATNLADGVPSRFSGSGSGTDFTLTISSLQPEDFATYYCQGIWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 8 aa, Homo sapiens
GFTFSSYV

SEQ ID NO: 413 - protein, 10 aa, Homo sapiens
IRLETHGYAA

SEQ ID NO: 414 - protein, 12 aa, Homo sapiens
ARVERSKSGFDN

SEQ ID NO: 415 - protein, 5 aa, Homo sapiens
SYVVH

SEQ ID NO: 416 - protein, 19 aa, Homo sapiens
RIRLETHGYAAEYAASVKG

SEQ ID NO: 417 - protein, 10 aa, Homo sapiens
VERSKSGFDN

SEQ ID NO: 418 - protein, 121 aa, Homo sapiens
QVQLVESGGGLVKPGGSLRLSCAASGFTFSSYVVHWVRQAPGKGLEWVGRIRLETHGYAAEYAASVKGRFTISRDDSKNTLYLQMNSLKTEDTAVYYCARVERSKSGFDNWGQGTLVTVSS

SEQ ID NO: 419 - protein, 451 aa, Homo sapiens
QVQLVESGGGLVKPGGSLRLSCAASGFTFSSYVVHWVRQAPGKGLEWVGRIRLETHGYAAEYAASVKGRFTISRDDSKNTLYLQMNSLKTEDTAVYYCARVERSKSGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 9 aa, Homo sapiens
SSNIGAGYS

SEQ ID NO: 421 - skipped

SEQ ID NO: 422 - protein, 11 aa, Homo sapiens
QSWDSSQTLVV

SEQ ID NO: 423 - protein, 14 aa, Homo sapiens
TGSSSNIGAGYSVH

SEQ ID NO: 424 - protein, 7 aa, Homo sapiens
GQSERPS

SEQ ID NO: 425 - protein, 11 aa, Homo sapiens
QSWDSSQTLVV

SEQ ID NO: 426 - protein, 111 aa, Homo sapiens
QSVLTQPPSVSGAPGQRVTISCTGSSSNIGAGYSVHWYQQLPGTAPKLLIYGQSERPSGVPDRFSGSKSGTSASLAITGLQAEDEADYYCQSWDSSQTLVVFGGGTKLTVL

SEQ ID NO: 427 - protein, 217 aa, Homo sapiens
QSVLTQPPSVSGAPGQRVTISCTGSSSNIGAGYSVHWYQQLPGTAPKLLIYGQSERPSGVPDRFSGSKSGTSASLAITGLQAEDEADYYCQSWDSSQTLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 428 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 429 - protein, 330 aa, Homo sapiens [sequence omitted]

SEQ ID NO: 430 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 431 - protein, 330 aa, Homo sapiens [sequence omitted]

SEQ ID NO: 432 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 433 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 434 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 435 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 436 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 437 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - DNA, 978 bp, Homo sapiens
  source 1..978: mol_type = unassigned DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 439 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 440 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 441 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 442 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 443 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 444 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 445 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 446 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 447 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 448 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 449 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 450 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 451 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 452 - DNA, 981 bp, Homo sapiens
  source 1..981: mol_type = unassigned DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 453 - DNA, 981 bp, Homo sapiens
  source 1..981: mol_type = unassigned DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 454 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 455 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 456 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 457 - DNA, 321 bp, Homo sapiens
  source 1..321: mol_type = unassigned DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 458 - protein, 107 aa, Homo sapiens [sequence omitted]

SEQ ID NO: 459 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 460 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 461 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 462 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 463 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 464 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 465 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 466 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 467 - DNA, 312 bp, Homo sapiens
  source 1..312: mol_type = unassigned DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 468 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 469 - DNA, 318 bp, Homo sapiens
  source 1..318: mol_type = unassigned DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 470 - DNA, 318 bp, Homo sapiens; identical to SEQ ID NO: 469
  source 1..318: mol_type = unassigned DNA

SEQ ID NO: 471 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 472 - DNA, 318 bp, Homo sapiens
  source 1..318: mol_type = unassigned DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 473 - DNA, 318 bp, Homo sapiens
  source 1..318: mol_type = unassigned DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 474 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 475 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 476 - protein, 106 aa, Homo sapiens; identical to SEQ ID NO: 474

SEQ ID NO: 477 - DNA, 312 bp, Homo sapiens
  source 1..312: mol_type = unassigned DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 478 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 479 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 480 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 481 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 482 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 483 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 484 - protein, 106 aa, Homo sapiens; identical to SEQ ID NO: 474

SEQ ID NO: 485 - DNA, 318 bp, Homo sapiens
  source 1..318: mol_type = unassigned DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 486 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 487 - DNA, 318 bp, Homo sapiens
  source 1..318: mol_type = unassigned DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 488 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 489 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 490 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 491 - protein, 506 aa, Rattus rattus
MPGLGRRAQWLCWWWGLLCSCGPPPLRPPLPVAAAAAGGQLLGAGGSPVRAEQPPPQSSSSGFLYRRLKTHEKREMQKEILSVLGLPHRPRPLHGLQQPQSPVLPQQQQSQQTAREEPPPGRLKSAPLFMLDLYNSLSKDDEEDGVSEGEGLEPESHGRASSSQLKQPSPGAAHSLNRKSLLAPGPGGSASPLTSAQDSAFLNDADMVMSFVNLVEYDKEFSPRQRHHKEFKFNLSQIPEGEAVTAAEFRVYKDCVVGSFKNQTFLISIYQVLQEHQHRDSDLFLLDTRVVWASEEGWLEFDITATSNLWVVTPQHNMGLQLSVVTRDGLHINPRAAGLVGRDGPYDKQPFMVAFFKVSEVHVRTTRSASSRRRQQSRNRSTQSQDVSRGSSASDYNSSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 492 - protein, 509 aa, Macaca fascicularis
MPGLGRRAQWLCWWWGLLCSCCGPPRPPLPAAAAAAAGGQLLGDGGSPGRTEQPPPSPQSSSGFLYRRLKTHEKREMQKEILSVLGLPHRPRPLHGLQQPQPPALPQQQQQQQQPPRGEPPPGRLKSAPLFMLDLYNALSADDEEDGASEGERQQPWPHEGASSSQPRQPAPGAAHPLNRKSLLAPGPGSGGASPLTSAQDSAFLNDADMVMSFVNLVEYDKEFSPRQRHHKEFKFNLSQIPEGEAVTAAEFRIYKDCVMGSFKNQTFLISIYQVLQEHQHRDSDLFLLDTRVVWASEEGWLEFDITATSNLWVVTPQHNMGLQLSVVTRDGVHIHPRAAGLVGRDGPYDKQPFMVAFFKVSEVHVRTTRSASGRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 493 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 494 - protein, 116 aa, Homo sapiens
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 495 - protein, 138 aa, Homo sapiens
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 496 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 497 - protein, 110 aa, Homo sapiens
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 498 - DNA, 1335 bp, Homo sapiens
  source 1..1335: mol_type = unassigned DNA
caggtccagtttgtgcagtctggggctgaggtgaagagtcctggggcctctgtgaaggtttcctgcaaggcttctggatacaccttcacaaatcatgctatacattgggtgcgccaggcccccggacaaaggcttgagtggatgggatggatcaacgctggcaatggtaaaactgattattcacagaacttccagggcagagtcatcattaccagggacacatccgcgaacacagcctacatgtccctgagcagcctgacatctgaggacacggctttttattactgtactagaagggtctacggtgaatcgtatgaccactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 499 - DNA, 642 bp, Homo sapiens
  source 1..642: mol_type = unassigned DNA
gacatccagatgacccagtctccatcctccctgtctgcatctgtaggagacagagtcaccatcacttgccgggcaagtcagggcattagaaataatttaggctggtatcagcagaaaccagggaaagcccctaagcgcctgatctatgctgcatccagtttgcaaagtggggtcccatcaaggtttagcggcagtggatctgggacagaattcactctcacaatcagcagcctgcagcctgaagatgttgcaatttatttctgtctacaacatcaaatttacccgtggacgttcggccaagggaccaaggtggaaaccaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 500 - DNA, 1335 bp, Homo sapiens
  source 1..1335: mol_type = unassigned DNA
caggtccaacttgtgcagtctggggctgaggtgcagaagcctggggcctcagtgaaggtttcctgcaaggcttctggatacaccttcacttcatatgctttgcattgggtgcgccaggcccccggacaaaggcttgagtggctgggatggatcagcgctgccaatggtaacacagattattcatggaagttccagggcagagtcacccttaccagggacacatccgcaaacacagtctacatggaactgaacagtctgacatctgaggactcggctgtgtattactgtgcgagaaggaaactatggtctccttttgatatctggggccaagggacattggtcaccgtctcttcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 501 - DNA, 645 bp, Homo sapiens
  source 1..645: mol_type = unassigned DNA
gaaattgtgttgacgcagtctccaggcaccctgtctttgtctccaggggaaagagccaccctctcctgttgggccagtcaaagtcttaccaacagtttcttagcctggtaccggcagaaacctggccaggctcccaggctcctcatctctggtgcattcagcagggccactgacatcccagacaggatcagtggcagtggatctgggacagacttcactctcaccatcaacagactggagcctgaagattttgcagtgtattactgtcagtactatggtacctcaccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 502 - DNA, 1335 bp, Homo sapiens
  source 1..1335: mol_type = unassigned DNA
caggtccaccttgttcagtcaggggcagaggtgaagaaccctggggcctcagtgaaggtctcctgcaaggcttctggatacaccttcactacctttgctattcattggttgcgccaggcccccggacagaggcttgagtggatgggatggatcaaccctggcaatgttaagacagattattcgcagaagttccagggcagagtcaccattagcagggacacatccgcgaccactgcctacatggagctgagcagcctgagatctgaagacacggctgtttattactgtgcgagaagacaattatggttaccctttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 503 - DNA, 645 bp, Homo sapiens
  source 1..645: mol_type = unassigned DNA
gaaattgtgttgacgcagtctccaggcaccctgtctttgtctccaggggagggagccaccctctcctgcagggccagtcagagtattagcaacaacttcttagcctggtaccaacagaaacctggccaggctcccagactcctcatctttggtgcatccagcagggccactgccatcccagacaggttcgttggcagtgggtctgggacagacttcactctcaccatcaccggactggagcctgaagattttgcagtgtatcactgtcaacactatggtggttcaccttggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 504 - DNA, 1335 bp, Homo sapiens; identical to SEQ ID NO: 502
  source 1..1335: mol_type = unassigned DNA

SEQ ID NO: 505 - DNA, 645 bp, Homo sapiens; identical to SEQ ID NO: 503
  source 1..645: mol_type = unassigned DNA

SEQ ID NO: 506 - DNA, 1335 bp, Homo sapiens
  source 1..1335: mol_type = unassigned DNA
caggtccagcttgtgcagtctggggctgaggtgaagaagcctggggcctcagtgaaagtttcctgtaaggcttctgggtacagtttcactaactatgctttacattgggtgcgccaggcccccggacaaagacttgagtggatgggatggatccacgctggtaatggtaagacagaatatgcacagaagttccaggacagagtcaccattagtagggacatatccgcgatcacagtttacatggaactgagcagcctgagatctgaagacacggctgtttattattgtgcgagaagacagttatggttaccctttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 507 - DNA, 645 bp, Homo sapiens
  source 1..645: mol_type = unassigned DNA
gaaattgtgttgacgcagtctccagacaccctctctttgtctccaggggaaacagccagtttctcctgcagggccggtcaaattattatcaacagacagttagcctggtaccagcggagacctggccaggctccccggctcctcatctatggcgcgtccaatagggtcactggcatcccagacaggttcagtggcagtgggtctgggacagacttcactctcacgatcaatagactggagcctgaagattttgcagtgtattactgtcagcactatggtggctcaccttggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 508 - DNA, 1335 bp, Homo sapiens
  source 1..1335: mol_type = unassigned DNA
caggtccagtttgtgcagtctggggctgaggtgaagaagcctggggcctcagtgaaggtttcctgtaaggcttctggatacaccttcactagccatgctatgcattgggtgcgccaggcccccggacaaaggcttgagtggatgggatggatcaacgctgccaatggtaaaacagattattcacagaacttccagggcagagtcaccattaccagggacacatacgcgaacacagtctacatggaactgagcagcctgagatctgaagacacggctgtgtattactgtgcgagacgcccttacggtggtccctttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 509 - DNA, 642 bp, Homo sapiens
  source 1..642: mol_type = unassigned DNA
gacatccagatgacccagtctccttccaccctgtctgcatctgtaggagacagagtcaccatcacttgccgggccagtcagagtattagtatctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgtcaacagtataatctttatccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 510 - DNA, 1335 bp, Homo sapiens; identical to SEQ ID NO: 508
  source 1..1335: mol_type = unassigned DNA

SEQ ID NO: 511 - DNA, 642 bp, Homo sapiens; identical to SEQ ID NO: 509
  source 1..642: mol_type = unassigned DNA

SEQ ID NO: 512 - DNA, 1332 bp, Homo sapiens
  source 1..1332: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtagtaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 513 - DNA, 645 bp, Homo sapiens
  source 1..645: mol_type = unassigned DNA
gaaattgtgttgacgcagtctccaggcaccctgtctttgtctccaggggaaagagccaccctctcttgttgggccagtcagagggttgtttacagatacttagcctggtaccagcggaaacctggccaggctcccagacttctcatttatggtgcattcaacagggccactggcatcccagacaggttcagtggcagtgggtctgggacagacttcagtctcactatcagtagactggagcctgaggattttgcagtttattactgtcaccaatatggtagttcaccaccgacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 514 - DNA, 1332 bp, Homo sapiens
  source 1..1332: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccatggactccctgtacctgcagatgaactccctgagagccgaggacaccgccgtgtactactgcgcccgtagatcctctggatggtacgactattggggccagggcaccctggtcacagtgtcctctgccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 515 - DNA, 645 bp, Homo sapiens
  source 1..645: mol_type = unassigned DNA
gaaattgtgctgactcagtcccctggcacactgtctttgagccctggcgagagagctaccctgtcttgttgggcctctcagagagtggtgtacagatacctggcttggtatcagcggaagcccggccaggctcctagactgttgatctacggcgccttcaacagagccacaggcatccctgacagattctccggctctggctctggcaccgacttctccctgactatctctcggctggaacccgaggacttcgccgtgtactactgtcaccagtacggcagcagccctcctacctttggccagggcactaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 516 - DNA, 1332 bp, Homo sapiens
  source 1..1332: mol_type = unassigned DNA
caggttcagctggttgaatctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccatggactccctgtacctgcagatgaactccctgagagccgaggacaccgccgtgtactactgcgcccgtagatcctctggatggtacgactattggggccagggcaccctggtcacagtgtcctctgcttctaccaagggacccagcgtgttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtcctgctccagagtttgaaggcggcccttccgtgtttctgttccctccaaagcctaaggacaccctgatgatctctcggacccctgaagtgacctgcgtggtggtggatgtgtcccaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatcagcaaggccaagggccagcctcgagaaccccaggtttacaccctgcctccaagccaagaggaaatgaccaagaaccaggtgtccctgacctgcctcgtgaagggattctacccctccgatatcgccgtggaatgggagtctaatggccagccagagaacaactacaagacaacccctcctgtgctggactccgacggctccttctttctgtattcccgcctgaccgtggacaagtccagatggcaagagggcaacgtgttctcctgcagcgtgatgcacgaggccctgcacaatcactacacccagaagtccctgtctctgtccctgggcaag

SEQ ID NO: 517 - DNA, 645 bp, Homo sapiens
  source 1..645: mol_type = unassigned DNA
gagatcgtgctgacccagtctcctggcacactgtcactgtctccaggcgagagagctaccctgtcttgttgggcctctcagagagtggtgtacagatacctggcttggtatcagcggaagcccggccaggctcctagactgttgatctacggcgccttcaacagagccacaggcatccctgacagattctccggctctggctctggcaccgacttctccctgactatctctcggctggaacccgaggacttcgccgtgtactactgtcaccagtacggcagcagccctcctacctttggccagggcacaaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 518 - DNA, 1332 bp, Homo sapiens
  source 1..1332: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtagtaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctcagcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 519 - DNA, 1332 bp, Homo sapiens
  source 1..1332: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtagtaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctcagcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 520 - DNA, 351 bp, Homo sapiens
  source 1..351: mol_type = unassigned DNA
caggttcagctggttgaatctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccatggactccctgtacctgcagatgaactccctgagagccgaggacaccgccgtgtactactgcgcccgtagatcctctggatggtacgactattggggccagggcaccctggtcacagtgtcctct

SEQ ID NO: 521 - DNA, 351 bp, Homo sapiens
  source 1..351: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccatggactccctgtacctgcagatgaactccctgagagccgaggacaccgccgtgtactactgcgcccgtagatcctctggatggtacgactattggggccagggcaccctggtcacagtgtcctct

SEQ ID NO: 522 - DNA, 324 bp, Homo sapiens
  source 1..324: mol_type = unassigned DNA
gagatcgtgctgacccagtctcctggcacactgtcactgtctccaggcgagagagctaccctgtcttgttgggcctctcagagagtggtgtacagatacctggcttggtatcagcggaagcccggccaggctcctagactgttgatctacggcgccttcaacagagccacaggcatccctgacagattctccggctctggctctggcaccgacttctccctgactatctctcggctggaacccgaggacttcgccgtgtactactgtcaccagtacggcagcagccctcctacctttggccagggcacaaaggtggaaatcaag

SEQ ID NO: 523 - DNA, 324 bp, Homo sapiens
  source 1..324: mol_type = unassigned DNA
gaaattgtgctgactcagtcccctggcacactgtctttgagccctggcgagagagctaccctgtcttgttgggcctctcagagagtggtgtacagatacctggcttggtatcagcggaagcccggccaggctcctagactgttgatctacggcgccttcaacagagccacaggcatccctgacagattctccggctctggctctggcaccgacttctccctgactatctctcggctggaacccgaggacttcgccgtgtactactgtcaccagtacggcagcagccctcctacctttggccagggcactaaggtggaaatcaaa

SEQ ID NO: 524 - DNA, 1335 bp, Homo sapiens
  source 1..1335: mol_type = unassigned DNA
caggtccaacttgtgcagtctggggctgaggtgaagaagcctggggcctcagtgaaggtttcctgcaaggctactggattcaccttcattacctatgctttccattgggtgcgccaggcccccggacaaaggtttgagtggatgggatggatcaacgttggcaatggtaacagagaatattcacagaagttccaggacagagtcaccattaccagggacacatccgcgaccacagtctacatggaactgaacagcctgaaatctgaagacacggctatgtatttctgtgcgagacgccccctctggggtccctttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 525 - DNA, 645 bp, Homo sapiens
  source 1..645: mol_type = unassigned DNA
gaaattgttttgacacagtctccaggcaccctgtctttgtctccaggggaaagagccaccctctcctgcagggccagtcagatttttagcaacaccttcttagcctggtaccagcagaaacctggccaggctcccaggctcctcgtgtatggtgcatccaagagggccactgccatcccagacaggtttagtggcagtgggtctgggacagacttcattctcaccatcaacagactggagcctgaagattttgcagtatattactgtcaacactatggtgggtcaccgtggacgttcggccgagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 526 - DNA, 1335 bp, Homo sapiens; identical to SEQ ID NO: 524
  source 1..1335: mol_type = unassigned DNA

SEQ ID NO: 527 - DNA, 645 bp, Homo sapiens; identical to SEQ ID NO: 525
  source 1..645: mol_type = unassigned DNA

SEQ ID NO: 528 - DNA, 1335 bp, Homo sapiens
  source 1..1335: mol_type = unassigned DNA
caggtccagcttgtgcagtctggggctgaggtgaagaagcctggggcctcagtgaaggtttcctgcaaggcttctggatacaccttcactagccatgctatacattgggtgcgccaggcccccggacaacggcttgagtggatgggatggatccacgctggcaatggtaactcaaaacagtcacagaacttccaggacagagtcaccattaccagggacacatccgcgagcgcagcctacatggagctgagcagcctgagatctgaagacacggctgtatattactgtgcgagacgggccataatggccccgtttgacctctggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 529 - DNA, 639 bp, Homo sapiens
  source 1..639: mol_type = unassigned DNA
gacatccagatgacccagtctccttccaccctgtctgcatctgtaggagacagagtcaccatcacttgccgggccagtcagagtattaataactggttggcctggtatcagcagaaaccagggaaagcccctaacctcctgatctataaggcgtctagtttagaaagtggggtcccatcaaggttcagcggcagtggatctgggacagaattcactctcaccatcaacagcctgcagcctgatgactttgcaacttattactgccaacagtattatagttcttggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 530 - DNA, 1335 bp, Homo sapiens
  source 1..1335: mol_type = unassigned DNA
caggtccagcttgtgcagtctggggctgaggtgaagaagcctggggcctcagtgaaggtttcctgcaaggcttctggatacaccttcactagctatgctgtccattgggtgcgccaggcccccggacaaaggcttgagtggatgggatggatcaacgctggcaatggtaacacaaaattttcacagaagttccagggcagaatcaccattaccagggacacatccgcgagcacaacctacatggagttgaatagtctgagatctgaagacacggctgtgtattattgtgcgagaagggggttcggggagccatttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 531 - DNA, 642 bp, Homo sapiens
  source 1..642: mol_type = unassigned DNA
gaaatagtgatgacgcagtctccagccaccctgtctgtgtctccaggggaaagagccaccctctcctgcagggccagtcagagtattagtaacaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 532 - DNA, 1335 bp, Homo sapiens
  source 1..1335: mol_type = unassigned DNA
caggtccagcttgtgcagtctggggctgaggtgaagaagcctggggcctcagtgaaggtttcctgcaaggcttctggatacaccttcactagctatgctatccattgggtgcgccaggcccccggacaaaggcttgagtggatgggatggatcaaccctggcaatggtaacacaaaattttcacagaagttccagggcagaatcaccattaccagggacacatccgcgagcacaacctacatggagctgaacagcctgagatctgaagacacggctgtgtattactgtgcgagaagggggttcggggagccatttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 533 - DNA, 642 bp, Homo sapiens
  source 1..642: mol_type = unassigned DNA
gaaatagtgatgacgcagtctccagccaccctgtctgtgtctccaggggaaagagccaccctctcctgcagggccagtcagagtattagcagcaacttagcctggtaccaacagaaccctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 534 - DNA, 1341 bp, Homo sapiens
  source 1..1341: mol_type = unassigned DNA
caggtccaacttgtgcagtctggggctgaggtgaagaagcctggggcctcagtgaaggtttcctgcaaggcttctggatacaccttcactagctatgctatgcattgggtgcgtcaggcccccggacacaggcttgagtggttgggatggatcaacgctggcaatggttacacaaaatattcacagaaattccaggacagagtcgccattaccagggacacatccgcgagcacagccttcatggagctgagcagcctgagatctgaagacacggctgtgtattactgtgcgagagatcgtattactattattcggccctttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 535 - DNA, 642 bp, Homo sapiens
  source 1..642: mol_type = unassigned DNA
gaaattgtgttgacgcagtctccaggcaccctgtctttgtctccaggggaaagagccaccctctcctgcagggccagtcagagtattagcagcagctacttagcctggttccagcaaaaacctggccaggctcccaggctcctcatctatggtgcatccagcagggccactggcatcccagacaggttcagtggcaatgggtctgggacagacttcactctcaccatcagcagactggagcctgaagattttgcagtgtattactgtcagctgtatggtagcccattcactttcggccctgggaccaaaatggatattaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 536 - DNA, 1335 bp, Homo sapiens
  source 1..1335: mol_type = unassigned DNA
caggtccagcttgtgcagtctggggctgaggtgaagaagcctggggcctcagtgaaagtttcctgcaaggcttctggatacaccttcactagctatgctatccattgggtgcgccaggcccccggacaaaggcttgagtggatgggatggatcaacgttggcaatggtaaaacaaaattttcacagaagttacagggcagaatcaccattaccagggacacatccgcgagcacaacctacatggagctgaacagcctgagatctgaagacacggctgtgttttactgtgcgagaagggggttcggggagccatttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 537 - DNA, 642 bp, Homo sapiens
  source 1..642: mol_type = unassigned DNA
gaaatagtgatgacgcagtctccagccaccctgtctgtgtctccaggggaaagagccaccctctcctgcagggccagtcagagtattagtagcaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 538 - DNA, 1335 bp, Homo sapiens
  source 1..1335: mol_type = unassigned DNA
caggtccagcttgtgcagtctggggctgaggtgaagaagcctggggcctcagtgaaggtttcctgcaaggcttctggatacaccttcactagctatgctatccattgggtgcgccaggcccccggacaaaggcttgagtggatgggatggatcaacgctggcaatggtaacacaaaatattcacagaagttccagggcagaatcaccattaccagggacacatccgcgagcacagcctacatggagctgagcagcctgagatctgaagacacggctgtgtattactgtgcgagaagggggttcggggagccatttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 539 - DNA, 642 bp, Homo sapiens
  source 1..642: mol_type = unassigned DNA
gaaatagtgatgacgcagtctccagccaccctgtctgtgtctccaggggaaagagccaccctctcctgcagggccagtcagagtattagcagcaacttagcctggtaccaacagaaacctggccaggctcccaggctcctcatctatggtacatccaccagggccactggtatcccagccaggttcagtggcagtgggtctgggacagagttcactctcaccatcagcaccctgcagtctgaagattttgcagtttattactgtcagcagtataatatctggcctttcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 540 - DNA, 1335 bp, Homo sapiens
  source 1..1335: mol_type = unassigned DNA
caggtccaccttgtgcagtctggggctgaggtgaagaagcctggggcctcagtgaaggtgtcctgcaagacttctggatacaccttcaccacctatgctattcattgggtgcgccaggcccccggacaagggcttgagtggatgggatggatcaacgctggcaatggtagaacagaatattcagagaagtttcagggcagagtcaccattaccagggacacttccgcgagtacagtctacatggacctgagcagcctgagatctggagacacggctgtgtattactgtgcgagaaggggattcggggagccatttgaccaatggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 541 - DNA, 642 bp, Homo sapiens
  source 1..642: mol_type = unassigned DNA
gaaatagtgatgacgcagtctccagccaccctgtctgtgtctccaggggaaagagccaccctctcctgcagggccagtcagagtgttagcagcaacttagcctggtaccagcaccaacctggccaggctcccaggctcctcatctatggtgcatccaccagggccactggtttcccacccaggttcagtggcagtgggtctgggacagacttcactctcaccatcaacagcctgcagtctgaagattttgcagtttattactgtcagcagtataataactggcctttcattttcggccctgggaccaaactggatatcacacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 542 - DNA, 1332 bp, Homo sapiens
  source 1..1332: mol_type = unassigned DNA
caggtgcaactggtggagtctgggggaggcttggtcaagcctggagggtccctgagactctcctgtgcagcctctggattcaccttcagtgactactacatgagctggatccgccaggctccagggaaggggctggagtgggtttcatacattagtattagtggtagtaccatatactacgcagactctgtgaagggccgattcaccatctccagggacaacgccaagaactcactgtttctgcaaatgaacagcctgagagccgaggacacggccgtgtattactgtgcgagaagaagcagtggctggtacgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 543 - DNA, 645 bp, Homo sapiens
  source 1..645: mol_type = unassigned DNA
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccatcagggccactggcatcccagacaggttcagtggcagtgggtctgggacagacttcactctcaccatcagcagactggagcctgaagattttgcagtgtattcctgtcaccagtatggtagctcacctccgacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 544 - DNA, 1332 bp, Homo sapiens
  source 1..1332: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccaagaactccctgttcctgcagatgaacagcctgagagccgaggacaccgccgtgtactactgcgcccgtagatcctctggatggtacgactattggggccagggcaccctggtcacagtttctagtgccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 545 - DNA, 645 bp, Homo sapiens
  source 1..645: mol_type = unassigned DNA
gaaattgtgctgactcagtcccctggcacactgtctttgagccctggcgagagagctaccctgtcctgtagagcctctcagtccgtgtcctccaactacctggcctggtatcagcagaagcctggacaggctccccggctgttgatctacggcgcttctatcagagccacaggcatccctgaccggttctccggatctggctctggcaccgatttcaccctgaccatctctcggctggaacccgaggatttcgccgtgtactcttgccaccagtacggctctagccctcctacctttggacagggcaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 546 - DNA, 1332 bp, Homo sapiens
  source 1..1332: mol_type = unassigned DNA
caggtgcaactggtggagtctgggggaggcttggtcaagcctggagggtccctgagactctcctgtgcagcctctggattcaccttcagtgactactacatgagttggatccgccaggctccagggaaggggctggagtgggtttcatacattagtattagtggtactaccatatactacgcagactctgtgaagggccgattcaccatctccagggacaacgccatggactcactgtatctgcaaatgaacagcctgagagccgaggacacggccgtgtattactgtgcgagacggagcagtggctggtacgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 547 - DNA, 645 bp, Homo sapiens; identical to SEQ ID NO: 513
  source 1..645: mol_type = unassigned DNA

SEQ ID NO: 548 - DNA, 1332 bp, Homo sapiens
  source 1..1332: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggcaccaccatctactacgccgactccgtgaagggcagattcaccatctccagagacaacgccatggactccctgtacctgcagatgaactccctgagagccgaggacaccgccgtgtactactgcgcccgtagatcctctggatggtacgactattggggccagggcaccctggtcacagtttctagtgccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 549 - DNA, 645 bp, Homo sapiens; identical to SEQ ID NO: 515
  source 1..645: mol_type = unassigned DNA

SEQ ID NO: 550 - DNA, 1332 bp, Homo sapiens
  source 1..1332: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtattagtggtagtaccatatactacgcagactctgtgaagggccgattcaccatctccagggacaacgccaagaactcactgtatctgcaaatgaacagcctgagagccgaggacacggccgtgtattactgtgcgagaagaagcagtggctggtacgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 551 - DNA, 645 bp, Homo sapiens
  source 1..645: mol_type = unassigned DNA
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccatcagggccactggcatcccagacaggttcagtggcagtgggtctgggacagacttcactctcaccatcagcagactggagcctgaagattttgcagtgtattgctgtcaccagtatggtagctcacctccgacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 552 - DNA, 1332 bp, Homo sapiens
  source 1..1332: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggctccaccatctactacgccgactccgtgaagggcagattcaccatctccagagacaacgccaagaactccctgtacctgcagatgaacagcctgagagccgaggacaccgccgtgtactactgcgcccgtagatcctctggatggtacgactattggggccagggcaccctggtcacagtttctagtgccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 553 - DNA, 645 bp, Homo sapiens
  source 1..645: mol_type = unassigned DNA
gaaattgtgctgactcagtcccctggcacactgtctttgagccctggcgagagagctaccctgtcctgtagagcctctcagtccgtgtcctccaactacctggcctggtatcagcagaagcctggacaggctccccggctgttgatctacggcgcttctatcagagccacaggcatccctgaccggttctccggatctggctctggcaccgatttcaccctgaccatctctcggctggaacccgaggatttcgccgtgtactgctgtcaccagtacggctctagccctcctacctttggacagggcaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 554 - DNA, 1332 bp, Homo sapiens
  source 1..1332: mol_type = unassigned DNA
caggtgcagctggtggagtctgggggaggcttggtcaagcctggagggtccctgcgactctcctgtgcagcctctggattcaccttcagtgactacttcatgagctggatccgccaggctccagggaaggggctggagtgggtttcgtacattagtattagtggtagtaccatatactacgcagactctgtgaagggccgattcaccatctccagggacaacgccaggaactcactgtttctgcaaatgaacagcctgagagccgaggacacggccatctattactgtgcgagaagaaccagtggctggtacgacttctggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 555 - DNA, 645 bp, Homo sapiens
  source 1..645: mol_type = unassigned DNA
gaaattgtgttgacgcagtctccaggcaccctgtctttgtctccaggggaaagagccaccctctcctgcagggccagtcagagtgttagctacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgttgtcaccagtatggtagttcacctccgacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 556 - DNA, 1332 bp, Homo sapiens
  source 1..1332: mol_type = unassigned DNA
caggtgcagctggtggagtctgggggaggcttggtcaagcctggagggtcccttagactctcctgtgcagcctctggattcaccttcagtgacttctacatgagctggatccgccaggctccagggagggggctggagtgggtttcgtacattagtattagtggtactaccatatactacgcagactctgtgaagggccgattcaccatctccagggacaacgccaggaactcactgtatctgcaaatgaacagcctgagagccgaggacacggccgtgtattactgtgcgagaagaaccagtggctggtacgacttctggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 557 - DNA, 645 bp, Homo sapiens
  source 1..645: mol_type = unassigned DNA
gaaattgtgttgacgcagtctccaggcaccctgtctttgtctccaggggaaagagccaccctctcctgcagggccagtcagagtgttagctacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgctgtcaccagtatggtagttcacctccgacgttcggccaagggaccaaggtggaaatgaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 558 - DNA, 1332 bp, Homo sapiens
  source 1..1332: mol_type = unassigned DNA
caggtgcagctggtggagtctgggggaggcttggtcaggcctggagggtccctgagactctcctgtgcagcctctggattcaccttcagtgactactacatgagctggatccgccaggctccagggaaggggctggagtgggtttcgtacattagtattagtggtagtaccatatactacgcagactctgtgaagggccgattcaccatctccagggacaacgccagggactcactttatctgcaaatgaacagcctgagagccgaggacacggccgtgtattactgtgcgagaagaaccagtggctggtacgacttctggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 559 - DNA, 645 bp, Homo sapiens
  source 1..645: mol_type = unassigned DNA
gaaattgtgttgacgcagtctccaggcaccctgtctttgtctccaggggaaagagccaccctctcctgtagggccagtcagagtgttagctacaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattgctgtcaccagtatggtagttcacctccgacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 560 - DNA, 1332 bp, Homo sapiens
  source 1..1332: mol_type = unassigned DNA
caagttcagttggttgagtctggcggcggactggttaagcctggcggatctctgagactgtcttgtgccgcctctggcttcaccttctccgactactacatgtcctggatcagacaggcccctggcaaaggcctggaatgggtgtcctacatctccatctccggcatcaccatctactacgccgactccgtgaagggcagattcaccatctccagagacaacgcccggaactccctgtacctgcagatgaactctctgagagccgaggacaccgccgtgtactactgcgcccgtagatcctctggatggtacgactattggggccagggcaccctggtcacagtttctagtgccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 561 - DNA, 645 bp, Homo sapiens
  source 1..645: mol_type = unassigned DNA
gaaattgtgctgactcagtcccctggcacactgtctttgagccctggcgagagagctaccctgtcctgtagagcctctcagtccgtgtcctacaactacctggcctggtatcagcagaagcccggccaggctcctagactgttgatctacggcgcctccatcagagccacaggcatccctgatagattctccggctctggctctggcaccgacttcaccctgaccatctccagactggaacccgaggacttcgccgtgtactgctgtcaccagtacggctctagccctcctacctttggacagggcaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 562 - protein, 139 aa, Homo sapiens
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 563 - protein, 139 aa, Rattus rattus
SASSRRRQQSRNRSTQSQDVSRGSSASDYNSSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 564 - protein, 139 aa, Macaca fascicularis
SASGRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 565 - protein, 118 aa, Homo sapiens
QVQLVQSGAEVKKPGSSVKVSCKASGYTFTSYAMHWVRQAPGQGLEWMGYINPYNRGTKYNENFKGRVTITADESTSTAYMELSSLRSEDTAVYYCARRPFGNAMDIWGQGTLVTVSS

SEQ ID NO: 566 - protein, 445 aa, Homo sapiens
QVQLVQSGAEVKKPGSSVKVSCKASGYTFTSYAMHWVRQAPGQGLEWMGYINPYNRGTKYNENFKGRVTITADESTSTAYMELSSLRSEDTAVYYCARRPFGNAMDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 567 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 568 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 569 - protein, 97 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPP

(19) United States
(12) Patent Application Publication
(10) Pub. No.: US 2026/0347455 A1
(43) Pub. Date: Nov. 5, 2026

(54) ANTAGONISTS

(71) Applicant: Kymab Limited, Cambridge (GB)

(72) Inventors: Volker GERMASCHEWSKI, Cambridge (GB); Igor THEURL, Cambridge (GB); Joana de Abreu CARVALHO, Cambridge (GB); Morgane Marie LECOINTRE, Cambridge (GB); Jonathan Leslie PAPWORTH, Cambridge (GB); Luke Thomas BAYLISS, Cambridge (GB); Verena PETZER, Cambridge (GB)

(21) Appl. No.: 19/721,681

(22) Filed: Jun. 26, 2026

Related U.S. Application Data
(63) Continuation of application No. 17/279,513, filed on Aug. 15, 2019, now Pat. No. 12,698,323.

(30) Foreign Application Priority Data
Sep. 25, 2018 (GB) 1815629.9

(51) Int. Cl. C07K 16/22; A61K 39/00; A61K 39/395; A61P 7/06

(52) U.S. Cl. CPC C07K 16/22; A61K 39/3955; A61P 7/06; A61K 2039/505; A61K 2039/545; C07K 2317/31; C07K 2317/33; C07K 2317/52; C07K 2317/55; C07K 2317/56; C07K 2317/565; C07K 2317/76; C07K 2317/92

(57) ABSTRACT

The invention relates to Bone Morphogenetic Protein 6 (BMP6) antagonists, such as antibodies and fragments, as well as methods, uses and combinations.

### CROSS REFERENCE TO RELATED APPLICATIONS

This application is a continuation of U.S. patent application Ser. No. 17/279,513, filed internationally on Aug. 15, 2019, which is a national stage application under 35 U.S.C. § 371 of International Application No. PCT/GB2019/052294, filed internationally on Aug. 15, 2019, which claims priority benefit to United Kingdom Application No. 1815629.9, filed Sep. 25, 2018, the contents of each of which are incorporated herein by reference in their entirety.

### REFERENCE TO AN ELECTRONIC SEQUENCE LISTING

The contents of the electronic sequence listing (165062000401SEQLIST.xml; Size: 601,863 bytes; and Date of Creation: Jun. 25, 2026) is herein incorporated by reference in its entirety.

### FIELD OF THE INVENTION

The invention relates to Bone Morphogenetic Protein 6 (BMP6) antagonists, such as antibodies and fragments, as well as methods, uses and combinations.

### BACKGROUND

Bone Morphogenetic Protein 6 (BMP6) is the key regulator of hepcidin, the small peptide secreted by the liver which is the major regulator of iron metabolism in mammals. Anti-BMP6 antagonists, such as antibodies, are being developed for use in a method of treating or preventing anaemia (see, eg, WO2016098079, US20160176956A1). See also WO2017191437 which discloses combinations of anti-BMP6 antagonists with erythropoietin stimulating agents (ESAs).

Anaemia is a major disease impacting 25% of the global population, or more than 1.7 billion people, particularly pregnant women, neonates and children. More than 40% of anaemia reflect a malfunction in the homeostatic control of iron uptake, storage and recycling. This dysregulation is a consequence of a variety of chronic diseases including infection (e.g. HIV, hepatitis), inflammation (e.g. rheumatoid arthritis), cancer and kidney disease. The enormous impact of diseases causing dysregulation of iron homeostasis can be seen in the USA where, of 40 million adults of >65 years of age, 10% suffer from anaemia and ⅓ of these are caused by chronic disorders.

Standards of care focus on blood transfusions and treatments with ESAs such as EPO or Aranesp® (Amgen, Inc).

The hamp gene encodes hepcidin, a 25 amino acid peptide hormone produced by the liver. Hepcidin works principally by controlling iron flux out of cells by regulating the amount of the iron transporter ferroportin present on the cell surface of cells involved in iron trafficking. Hepcidin interacts with ferroportin, primarily expressed on macrophages and duodenal enterocytes, causing internalisation and degradation of ferroportin (Ganz and Nemeth, 2011; Ramey et al., 2010). BMP6 triggers hepcidin expression in a mechanism involving several receptors and co-factors. These include BMP class I and class II receptors which are essential for triggering hamp expression and members of the repulsive guidance molecule family RGM such as RGMc (hemojuvelin, HJV) and RGMb (DRAGON), matriptase-2, neogenin, HFE and transferrin receptors 1 and 2. The SMAD pathway is presumably triggered by the initial interaction of BMP6 with class II BMP receptors possibly also involving HJV. This causes an association with the auto-phosphorylated BMPR II and activation of phosphorylation of BMPRI thus triggering of the SMAD cascade. Phosphorylated SMAD1, 5 and 8 and eventually SMAD4 translocate to the nucleus to interact with BMP responsive elements in the hamp gene control region to trigger expression of hepcidin. In mice, liver specific disruption of the SMAD4 signalling molecule or the type I receptors Alk2 and ALk3 decrease hepcidin expression similar to a BMP6 knock out confirming that these factors are also part of the relevant pathway (Steinbicker et al., 2011; Wang et al., 2005).
Ganz, T., Nemeth, E., 2011. The Hepcidin-Ferroportin System as a Therapeutic Target in Anemias and Iron Overload Disorders. Hematology 2011, 538-542.
Ramey, G., Deschemin, J. C., Durel, B., Canonne-Hergaux, F., Nicolas, G., Vaulont, S., 2010. Hepcidin targets ferroportin for degradation in hepatocytes. Haematologica 95, 501-504.
Steinbicker, A. U., Bartnikas, T. B., Lohmeyer, L. K., Leyton, P., Mayeur, C., Kao, S. M., Pappas, A. E., Peterson, R. T., Bloch, D. B., Yu, P. B., Fleming, M. D., Bloch, K. D., 2011. Perturbation of hepcidin expression by BMP type I receptor deletion induces iron overload in mice. Blood 118, 4224-4230.
Wang, R.-H., Li, C., Xu, X., Zheng, Y., Xiao, C., Zerfas, P., Cooperman, S., Eckhaus, M., Rouault, T., Mishra, L., 2005. A role of SMAD4 in iron metabolism through the positive regulation of hepcidin expression. Cell Metab. 2, 399-409.

### STATEMENT OF INVENTION

The invention provides the following:—

An antibody or fragment comprising a binding site which specifically binds to Bone Morphogenetic Protein 6 (BMP6), wherein the binding site comprises a VH domain, wherein the VH domain comprises a CDRH3 sequence of a VH domain comprising SEQ ID NO: 114.

An antibody or fragment comprising a binding site which specifically binds to BMP6, wherein the binding site comprises a VH domain that comprises SEQ ID NO: 114, or an amino acid that is at least 70% identical thereto.

An antibody or fragment comprising a binding site which specifically binds to BMP6, wherein the binding site comprises a VL domain, wherein the VL domain comprises a CDRL3 sequence of a VL domain comprising SEQ ID NO: 123.

An antibody or fragment comprising a binding site which specifically binds to BMP6, wherein the binding site comprises a VL domain that comprises SEQ ID NO: 123, or an amino acid that is at least 70% identical thereto.

An antibody or fragment which specifically binds to Bone Morphogenetic Protein 6 (BMP6) and comprises (i) a heavy chain amino acid sequence comprising SEQ ID NO: 116 or an amino acid that is at least 70% identical thereto; and/or (ii) a light chain sequence comprising SEQ ID NO: 125 or an amino acid that is at least 70% identical thereto.

An antibody or fragment which (i) specifically binds to a human BMP6 epitope that is identical to an epitope to which the antibody of the invention binds; and/or (ii) competes for binding to human BMP6 with the antibody of the invention.

The invention also provides the following configurations.

In a first configuration the invention provides:

An antibody or fragment comprising a binding site which specifically binds to Bone Morphogenetic Protein 6 (BMP6), wherein the binding site comprises a VH domain that is encoded by a nucleotide sequence that is derived from the recombination of a human VH gene segment, DH gene segment and JH gene segment, wherein the VH gene segment is selected from IGHV3-11 and IGHV1-3.

In a second configuration the invention provides:

An antibody or fragment which specifically binds to BMP6 and comprises the CDRH3 sequence of an anti-BMP6 antibody according to the invention, or said CDRH3 sequence comprising 3, 2 or 1 amino acid substitution(s).

In a third configuration the invention provides:

An antibody or fragment which specifically binds to BMP6 and comprises a VH domain which comprises a CDRH3 sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said sequence comprising 3, 2 or 1 amino acid substitution(s).

In a fourth configuration the invention provides:

An antibody or fragment comprising a binding site which specifically binds to BMP6, wherein the binding site comprises a VH domain that comprises the amino acid sequence of a VH domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70% identical thereto.

In a fifth configuration the invention provides:

An antibody or fragment comprising a binding site which specifically binds to BMP6, wherein the binding site comprises a VL domain that is encoded by a nucleotide sequence that is derived from the recombination of a human VL gene segment and JL gene segment, wherein the VL gene segment is selected from IGKV3-20, IGKV1-5 and IGKV3-15.

In a sixth configuration the invention provides:

An antibody or fragment which specifically binds to BMP6 and comprises the CDRL3 sequence of an anti-BMP6 antibody of the invention, said CDRL3 sequence comprising 3, 2 or 1 amino acid substitution(s).

In a seventh configuration the invention provides:

An antibody or fragment which specifically binds to BMP6 and comprises a VL domain which comprises a CDRL3 (and optionally a CDRH3) sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said sequence(s) each comprising 3, 2 or 1 amino acid substitution(s).

In a eighth configuration the invention provides:

An antibody or fragment comprising a binding site which specifically binds to BMP6, wherein the binding site comprises a VL domain that comprises the amino acid sequence of a VL domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70% identical thereto.

In a ninth configuration the invention provides:

An antibody or fragment which specifically binds to BMP6 and comprises the heavy chain amino acid sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70% identical thereto.

In a tenth configuration the invention provides:

An antibody or fragment which specifically binds to BMP6 and comprises the light chain amino acid sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70% identical thereto.

In a eleventh configuration the invention provides:

An antibody or fragment which specifically binds to a human BMP6 epitope that is identical to an epitope to which the antibody of the invention (eg, CL-58838) binds.

In a twelfth configuration the invention provides:

An antibody or fragment which competes for binding to human BMP6 with the antibody of the invention.

In a thirteenth configuration the invention provides:

An anti-BMP6 antibody or fragment of the invention for treating or preventing a BMP6-mediated disease or condition (optionally anaemia) in a subject.

In a fourteenth configuration the invention provides:

A combination of an amount of an anti-BMP6 antibody or fragment and an amount of an ESA (optionally comprising multiple doses of said antibody and/or ESA), wherein the antibody or fragment is according to the invention.

In a fifteenth configuration the invention provides:

Use of the antibody, fragment or combination of the invention in the manufacture of a medicament for administration to a subject for treating or preventing a BMP6-mediated disease or condition, optionally anaemia.

In a sixteenth configuration the invention provides:

A method of treating or preventing a BMP6-mediated disease or condition in a subject (optionally anaemia), the method comprising administering to said subject a therapeutically effective amount of an antibody, fragment or combination of the invention, wherein the BMP6-mediated disease or condition is thereby treated or prevented.

In a seventeenth configuration the invention provides:

A pharmaceutical composition comprising an antibody, fragment or combination of the invention and a pharmaceutically acceptable excipient, diluent or carrier.

In a eighteenth configuration the invention provides:

A nucleic acid that encodes a VH domain and/or a VL domain of an antibody or fragment of the invention

In a nineteenth configuration the invention provides:

A nucleic acid that encodes a VH domain comprising the amino acid sequence of a VH domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70% identical thereto.

In a twentieth configuration the invention provides:

A nucleic acid (eg, in a host cell, eg, a CHO or HEK293 or Cos cell) comprising
(a) a nucleotide sequence that is at least 70% identical to the sequence of SEQ ID NO: 115, 520 or 521; and/or
(b) a nucleotide sequence that is at least 70% identical to the sequence of SEQ ID NO: 124, 522 or 523.

A nucleic acid (eg, in a host cell, eg, a CHO or HEK293 or Cos cell) comprising
(a) a nucleotide sequence that is at least 70% identical to the sequence of SEQ ID NO: 115; and/or
(b) a nucleotide sequence that is at least 70% identical to the sequence of SEQ ID NO: 124.

A nucleic acid (eg, in a host cell, eg, a CHO or HEK293 or Cos cell) comprising
(a) a nucleotide sequence that is at least 70% identical to the sequence of SEQ ID NO: 520; and/or
(b) a nucleotide sequence that is at least 70% identical to the sequence of SEQ ID NO: 522.

A nucleic acid (eg, in a host cell, eg, a CHO or HEK293 or Cos cell) comprising
(a) a nucleotide sequence that is at least 70% identical to the sequence of SEQ ID NO: 521; and/or
(b) a nucleotide sequence that is at least 70% identical to the sequence of SEQ ID NO: 523.

In a twenty-first configuration the invention provides:

A combination of first and second nucleic acids (eg, in a host cell, eg, a CHO or HEK293 or Cos cell) respectively comprising
(a) a nucleotide sequence that is at least 70% identical to the sequence of SEQ ID NO: 115, 520 or 521; and/or
(b) a nucleotide sequence that is at least 70% identical to the sequence of SEQ ID NO: 124, 522 or 523.

A combination of first and second nucleic acids (eg, in a host cell, eg, a CHO or HEK293 or Cos cell) respectively comprising
(a) a nucleotide sequence that is at least 70% identical to the sequence of SEQ ID NO: 115; and/or
(b) a nucleotide sequence that is at least 70% identical to the sequence of SEQ ID NO: 124.

A combination of first and second nucleic acids (eg, in a host cell, eg, a CHO or HEK293 or Cos cell) respectively comprising
(a) a nucleotide sequence that is at least 70% identical to the sequence of SEQ ID NO: 520; and/or
(b) a nucleotide sequence that is at least 70% identical to the sequence of SEQ ID NO: 522.

A combination of first and second nucleic acids (eg, in a host cell, eg, a CHO or HEK293 or Cos cell) respectively comprising
(a) a nucleotide sequence that is at least 70% identical to the sequence of SEQ ID NO: 521; and/or
(b) a nucleotide sequence that is at least 70% identical to the sequence of SEQ ID NO: 523.

In a twenty-second configuration the invention provides:

A nucleic acid that encodes a heavy chain and/or a light chain of an antibody or fragment of the invention.

In a twenty-third configuration the invention provides:

A nucleic acid that encodes a heavy chain comprising an amino acid sequence that is at least 70% identical to SEQ ID NO: 116.

In a twenty-fourth configuration the invention provides:

A nucleic acid that encodes a light chain comprising an amino acid sequence that is at least 70% identical to SEQ ID NO: 125.

In a twenty-fifth configuration the invention provides:

A nucleic acid (eg, in a host cell, eg, a CHO or HEK293 or Cos cell) comprising
(a) a nucleotide sequence that is at least 70% identical to a heavy chain sequence selected of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; and/or
(b) a nucleotide sequence that is at least 70% identical to a sequence selected of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713.

A nucleic acid (eg, in a host cell, eg, a CHO or HEK293 or Cos cell) comprising
(a) a nucleotide sequence that is at least 70% identical to a sequence selected from SEQ ID NO: 512, 514, 516, 518 and 519; and/or
(b) a nucleotide sequence that is at least 70% identical to a sequence selected from SEQ ID NO: 513, 515 and 517.

A nucleic acid (eg, in a host cell, eg, a CHO or HEK293 or Cos cell) comprising
(a) a nucleotide sequence that is at least 70% identical SEQ ID NO: 512; and/or
(b) a nucleotide sequence that is at least 70% identical to SEQ ID NO: 513.

A nucleic acid (eg, in a host cell, eg, a CHO or HEK293 or Cos cell) comprising
(a) a nucleotide sequence that is at least 70% identical SEQ ID NO: 516; and/or
(b) a nucleotide sequence that is at least 70% identical to SEQ ID NO: 517.

A nucleic acid (eg, in a host cell, eg, a CHO or HEK293 or Cos cell) comprising
(a) a nucleotide sequence that is at least 70% identical SEQ ID NO: 518; and/or
(b) a nucleotide sequence that is at least 70% identical to SEQ ID NO: 513.

A nucleic acid (eg, in a host cell, eg, a CHO or HEK293 or Cos cell) comprising
(a) a nucleotide sequence that is at least 70% identical SEQ ID NO: 519; and/or
(b) a nucleotide sequence that is at least 70% identical to SEQ ID NO: 513.

A nucleic acid (eg, in a host cell, eg, a CHO or HEK293 or Cos cell) comprising
(a) a nucleotide sequence that is at least 70% identical SEQ ID NO: 518; and/or
(b) a nucleotide sequence that is at least 70% identical to SEQ ID NO: 517.

A nucleic acid (eg, in a host cell, eg, a CHO or HEK293 or Cos cell) comprising
(a) a nucleotide sequence that is at least 70% identical SEQ ID NO: 519; and/or
(b) a nucleotide sequence that is at least 70% identical to SEQ ID NO: 517.

A nucleic acid (eg, in a host cell, eg, a CHO or HEK293 or Cos cell) comprising
(a) a nucleotide sequence that is at least 70% identical SEQ ID NO: 514; and/or
(b) a nucleotide sequence that is at least 70% identical to SEQ ID NO: 515.

A nucleic acid (eg, in a host cell, eg, a CHO or HEK293 or Cos cell) comprising
(a) a nucleotide sequence that is at least 70% identical SEQ ID NO: 516; and/or
(b) a nucleotide sequence that is at least 70% identical to SEQ ID NO: 517.

A nucleic acid (eg, in a host cell, eg, a CHO or HEK293 or Cos cell) comprising
(a) a nucleotide sequence that is at least 70% identical SEQ ID NO: 518; and/or
(b) a nucleotide sequence that is at least 70% identical to SEQ ID NO: 513, 515 or 517.

A nucleic acid (eg, in a host cell, eg, a CHO or HEK293 or Cos cell) comprising
(a) a nucleotide sequence that is at least 70% identical SEQ ID NO: 519; and/or
(b) a nucleotide sequence that is at least 70% identical to SEQ ID NO: 513, 515 or 517.

In a twenty-sixth configuration the invention provides:

A vector comprising the nucleic acid(s); optionally wherein the vector is a CHO or HEK293 vector.

In a twenty-seventh configuration the invention provides:

A host cell comprising the nucleic acid(s) or the vector.

In a twenty-eighth configuration the invention provides:

An antibody, fragment, combination, vector, host cell, use or method as herein described.

In a twenty-ninth configuration the invention provides:

An antibody or fragment that specifically binds to a bone morphogenetic protein (BMP) for use in a method of treating or preventing a disease or condition caused by haemojuvelin (HJV)-deficient BMP-BMP receptor (BMPR) complexes in a human or animal subject, wherein the method comprises administering the antibody or fragment to the subject for inhibiting formation of said complexes and/or inhbiting triggering of intracellular signalling by such complexes in the subject, whereby a HJV-independent BMP-BMPR mediated disease or condition is treated or prevented.

An antibody or fragment that specifically binds to a bone morphogenetic protein (BMP) for use in a method of treating or preventing HJV-independent anaemia or osteoporosis in a human or animal subject, wherein the method comprises administering the antibody or fragment to the subject for inhibiting formation of haemojuvelin (HJV)-deficient BMP-BMP receptor (BMPR) complexes and/or inhbiting triggering of intracellular signalling by such complexes in the subject, whereby HJV-independent anaemia or osteoporosis is treated or prevented.

An antibody or fragment that specifically binds to a bone morphogenetic protein (BMP) for use in a method of treating or preventing haemojuvelin (HJV)-independent anaemia or osteoporosis in a human or animal subject, wherein the method comprises administering the antibody to the subject, whereby HJV-independent anaemia or osteoporosis is treated or prevented.

The invention also provides such methods for treating a disease or condition, eg, anaemia or osteoporosis.

### BRIEF DESCRIPTION OF THE DRAWINGS

FIGS. 1A-1B:

Establishing of the assay window using a HepG2 hamp luciferase reporter gene cell line and various human BMP ligands as stimulating agents. Red Firefly luciferase gene under control of the human hepcidin promoter regulatory element in HepG2 cells was tested for function by stimulating the cells with various human BMP proteins (R&D Systems or Peprotech) added at increasing concentrations. The performance of the assay was tested in two different culture media settings, MEM containing 1% FBS (FIG. 1A) and Hybridoma Media containing 25% MEM (FIG. 1B; details see Example 1).

FIG. 2:

HepG2 hamp luciferase reporter gene cell assay window assessed using various human or mouse BMP6 ligands as stimulating agents (R&D Systems or Peprotech) added at increasing concentrations. BMP6 was diluted in MEM containing 1% FBS. Total assay volume was 60 μl using 25% HMM (for more details and reagents used see Example 1).

FIGS. 3A-3B:

HepG2 hamp luciferase reporter gene cell assay window assessed using a fixed concentration of human (Peprotech) or mouse BMP6 (R&D Systems) at 1 nM in MEM containing 1% FBS and commercial mouse anti-BMP6 monoclonal antibodies MAB507 (FIG. 3A) and MAB2365 (FIG. 3B) from R&D Systems diluted in hybridoma maintenance medium (HMM); (for more details and reagents see Example 1).

FIG. 4:

Serum titer determination of immunisation regime KM089 using five bmp6−/− Kymice by a reverse DELFIA® assay (Perkin Elmer) where IgG antibody contained in the serum added at various serial dilutions was captured with anti-mouse IgG (goat anti-mouse IgG; Southern Biotech 1030-01) via the Fc-domain and then incubated with biotinylated BMP6 and detected using DELFIA Eu—N1 Europium-labelled streptavidin (Perkin Elmer). The cut-off (any signal below this value was judged as negative) was defined as the negative control average of all replicates+3x standard deviation. KMBM codes in legend refer to individual Kymouse™ bmp6−/− animals.

FIG. 5:

2 μg/lane purified human BMP6 (Peprotech) separated by SDS-PAGE and stained with Coomassie blue (A) under reducing (R) and non-reducing (NR) conditions and Western blots from such gels blotted onto membranes and then probed with purified human anti-BMP6 antibodies as labelled. Bound antibody was detected with anti-human kappa light chain-horse radish peroxidase (HRP) and peroxidase by Enhanced Chemiluminescence (CL-58838 & Antibody A) or with anti-human Fc-alkaline phosphatase (AP) and phosphatase 5-bromo-4-chloro-3-indolyl-phosphate (BCIP) and nitro blue tetrazolium (NBT) colorimetric substrate conversion (Antibody B).

FIGS. 6A-6B:

Receptor dimerization assay based on U2OS cells transfected with either BMPRIA (ALK3, CD292) and BMPRII (T-ALK; FIG. 6A) or BMPRIA (ALK6) and BMPRII (T-ALK; FIG. 6B). Ligand driven dimerization with BMP6 (Peprotech 120-06 SEQ ID NO: 2) generated a chemiluminescent signal. Human anti-BMP6 IgG4 antibody CL-58838 and anti-BMP6 Antibodies A, B (FIG. 6A), human anti-BMP6 IgG4 antibodies 1-8 (FIG. 6B) and murine monoclonal antibody MAB507 (R&D Systems; FIGS. 6A and B) were added at increasing concentrations (11-point dilutions). Representative data of two experiments in each case. Appropriate isotype control IgG were used in both experiments.

FIGS. 7A-7E:

Cross-reactivity profile of purified CL-58838 IgG4 (SEQ ID NO: 116 and SEQ ID NO: 125) with human BMP2 (FIG. 7A), BMP4 (FIG. 7B), BMP5 (FIG. 7C), BMP7 (FIG. 7D) and BMP9 (FIG. 7E; details described in Example 7). The effects of anti-BMP6 antibody CL-58838 on the BMP-driven activation of the hamp luciferase reporter gene in HepG2 cells was studied by adding increasing amounts of CL-58838. As a positive control, relevant BMP specific antibodies (all R&D Systems) were added to interfere with the relevant BMP activation.

FIGS. 8A-8I:

Plotted transferrin saturation in % (TSAT) in normal rats plotted from Table 12 (for FIGS. 8A-8D), Table 13 (for FIGS. 8 E-8G) and Table 14 (for FIGS. 8 H and 8I) following a single iv injection of anti-BMP-6 or isotype IgG control antibodies.

FIGS. 9A-9B:

Results for transferrin saturation in % (TSAT) in normal rats following a single iv injection of CL-58838 at various doses (FIG. 9A) or CL-58838 and Antibody A at 1 mg/kg (FIG. 9B; Table 15).

FIGS. 10A-10B:

Results for transferrin saturation in % (TSAT); in normal rats following a single sc injection of CL-58838 at various doses (FIG. 10A) or CL-58838 and Antibody A and B at 1 mg/kg (FIG. 10B; Table 16).

FIGS. 11A-11D:

Pharmacokinetic (PK) profile for human anti-BMP6 IgG4 antibody CL-58838 following a single iv injection at various doses (FIG. 11A) or comparison with Antibody A (FIG. 11B) at 1 mg/kg dose. PK profile following a single subcutaneous (sc) injection of CL-58838 at various doses (FIG. 11C) or Antibodies A and B at 1 mg/kg dose (FIG. 11D). Results plotted as IgG [ng/ml] IgG over time [h].

FIGS. 12A-12F:

Results of the rat PG-PS model of ACD. Transferrin saturation TSAT (FIG. 12A), Haemoglobin [g/dL] (FIG. 12B), MCH [pg] (FIG. 12C), and serum hepcidin levels [ng/ml] at week 0 (FIG. 12D), week 1 (FIG. 12E) and week 2 (FIG. 12F) after start of treatment (*p<0.05, **p<0.01, ***p<0.001).

FIGS. 13A-13D:

Transferrin saturation in % (TSAT) following a single iv administration of CL-58838 and Antibody A and B at 3 mg/kg in cynomolgus monkeys (FIG. 13A) and CL-58838 at various doses (FIG. 13B) and plasma hepcidin levels following a single iv administration of CL-58838 and Antibody A and B at 3 mg/kg in cynomolgus monkeys (FIG. 13C) and CL-58838 at various doses (FIG. 13D).

FIGS. 14A-14B:

PK profile of CL-58838 following a single iv injection in cynomolgus monkeys at various doses (A) and PK profile comparison of CL-58838 with Antibody A and B at 3 mg/kg (B).

FIG. 15:

Amino acid sequence alignment of antibody Vh and Vk sequences highly related to CL-58838 based on V-region usage and CDRH3 (Table 6). Top line shows the germline sequence of IMGT V-region genes IGHV3-11 and IGKV3-20 as encoded in bmp6−/− Kymouse used in this invention. Amino acid positions that differed from the germline V-region sequences in the selected antibody sequences below are boxed. Antibodies selected for in vivo assessment are shown in bold (Table 6).

FIG. 16:

Homogeneous time resolved FRET (HTRF) assay showing the ability of antibodies highly related to CL-58838 based on V-region usage and CDRH antibodies identified by NGS sequence (Table 6) to compete with labelled CL-58838 (10 nM) for binding to human BMP6 present at 32 nM. Competing antibodies were added as a range of concentrations in an 11-point dilution range starting at 3 μM final concentration.

FIGS. 17A-17F:

Experimental setup and results obtained from Example 17. Graphical illustration of experimental time course (FIG. 17A) decision diagram for inclusion of rats and application of EPO (FIG. 17B), Haemoglobin [g/dL] (FIG. 17C), MCV [fL] (FIG. 17D), MCH [pg] (FIG. 17E) and applied EPO doses [%] (FIG. 17F). ANCOVA analysis on change from baseline at week 4 with baseline as a covariate (FIGS. 17C-17E) and Fishers exact test (FIG. 17F) were used. Data are shown as mean±SEM (*p<0.05, ** p<0.01, ***p<0.001).

FIGS. 18A-18G:

Experimental setup and results for Example 18. Graphical illustration of experimental time course (FIG. 18A), Haemoglobin [g/dL] (FIG. 18B), MCV [fL] (FIG. 18C), MCH [pg] (FIG. 18D), hepatic Hamp mRNA levels [fold expression], Plasma iron levels [μg/dL] (FIG. 18F) and TSAT values [%]. Analysis of variance with Dunnett's correction for multiple comparisons vs. EPO was applied. Results for comparison of EPO vs. EPO+CL-58838 are indicated. Data are shown as mean±SEM (*p<0.05, ** p<0.01, ***p<0.001).

FIGS. 19A-19F:

Results obtained from Example 19. Haemoglobin values [g/dL] comparing treatment groups after administration of 1 μg/kg EPO in combination with different CL-58838 concentrations (FIG. 19A), Haemoglobin values [g/dL] after administration of 10 mg/kg CL-58838 in combination with different EPO doses (FIG. 19B), MCV values [fL] (FIG. 19C) and TSAT [%] (FIG. 19D) of mice after administration of 1 μg/kg EPO in combination with different CL-58838 concentrations, MCV values [fL] of mice after administration of 1 mg/kg CL-58838 in combination with different EPO doses (FIG. 19E), hepatic Hamp mRNA levels [fold expression] among all treatment groups (FIG. 19F). Analysis of variance with Dunnett's correction for multiple comparisons vs. Group II was applied. Data are shown as mean±SEM (*p<0.05, **p<0.01, ***p<0.001).

### DETAILED DESCRIPTION

### Definitions

Unless otherwise defined herein, scientific and technical terms shall have the meanings that are commonly understood by those of ordinary skill in the art. Further, unless otherwise required by context, singular terms shall include pluralities and plural terms shall include the singular. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” In the specification and claims, the term “about” is used to modify, for example, the quantity of an ingredient in a composition, concentration, volume, process temperature, process time, yield, flow rate, pressure, and like values, and ranges thereof, employed in describing the embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an anti-hBMP6 antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The term “antibody”, “immunoglobulin” or “lg” may be used interchangeably herein and means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies (including dual binding antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, hIgG2, mIgG2A, mIgG2B, IgG3, IgG4, IgA1 and IgA2. In mammals, there are two types of immunoglobulin light chains, λ and κ. The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. An example of antibodies are heavy chain-only (ie, H2) antibodies that comprise a dimer of a heavy chain (5′-VH-(optional Hinge)-CH2-CH3-3′) and are devoid of a light chain.

The antibodies described herein may be oligoclonal, polyclonal, monoclonal (including full-length monoclonal antibodies), camelised, chimeric, CDR-grafted, multi-specific, bi-specific (including dual-binding antibodies), catalytic, chimeric, humanized, fully human, anti-idiotypic, including antibodies that can be labelled in soluble or bound form as well as fragments, variants or derivatives thereof, either alone or in combination with other amino acid sequences provided by known techniques. An antibody may be from any species. Antibodies described herein can be naked or conjugated to other molecules such as toxins, radioisotopes, etc.

The term “antigen binding site,” “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g. the complementarity determining regions (CDRs)). The antigen binding region can be derived from any animal species, such as rodents (e.g. rabbit, rat or hamster) and humans. Preferably, the antigen binding region will be of human origin.

Antigen binding fragments described herein can include single-chain Fvs (scFv), single-chain antibodies, single domain antibodies, domain antibodies, Fv fragments, Fab fragments, F(ab′) fragments, F(ab′)₂ fragments, antibody fragments that exhibit the desired biological activity, disulfide-stabilised variable region (dsFv), dimeric variable region (diabody), anti-idiotypic (anti-Id) antibodies (including, e.g. anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognized by Fc receptors (FcR) found on certain types of cells. Digestion of antibodies with the enzyme, pepsin, results in a F(ab′)₂ fragment in which the two arms of the antibody molecule remain linked and comprise two-antigen binding sites. The F(ab′)₂ fragment has the ability to crosslink antigen.

The term “derived from the recombination of” in relation to gene segments will be readily apparent to the skilled person, who will understand that B-cells recombine their variable region gene segmements to produce coding sequence for variable domains. For example “derived from the recombination of a human VH gene segment, DH gene segment and JH gene segment” relates to the recombination of one human VH gene segment, with one DH gene segment and one JH gene segment together to form a rearranged VDJ sequence encoding a heavy chain antibody variable domain. Junctional and somatic hypermutation may also be features of the process, whereby the resulting recombined VDJ sequence includes one or more nucleotide additions, substitutions or deletions (eg, p-additions and/or n-additions) that are not comprised by the germline V, D and J sequences. The equivalent will be said of Vκ and Jκ gene segments for a kappa light chain variable domain, and of Vλ and Jλ for a lambda light chain variable domain. It is intended that any post-translational modifications may additionally encompassed in variable domains.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g. isomerizations, amidations) that may be present in minor amounts. Monoclonal antibodies are highly specific and are directed against a single antigentic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals. The monoclonal antibodies herein can include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies that exhibit the desired biological activity.

The term “humanised antibody” refers to a subset of chimeric antibodies in which a “hypervariable region” from a non-human immunoglobulin (the donor antibody) replaces residues from a hypervariable region in a human immunoglobulin (recipient antibody). In general, a humanized antibody will include substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more substitutions that improve antibody performance, such as binding affinity, isomerization, immunogenicity, etc.

The term “bispecific antibody” means an antibody which comprises specificity for two target molecules, and includes, but is not limited to, formats such as DVD-Ig (see DiGiammarino et al., “Design and generation of DVD-Ig™ molecules for dual-specific targeting”, Meth. Mo. Biol., 2012, 889, 145-156), mAb² (see WO2008/003103, the description of the mAb² format is incorporated herein by reference), FIT-Ig (see WO2015/103072, the description of the FIT-Ig scaffold is incorporated herein by reference), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. For a review of bispecific formats, see Spiess, C., et al., Mol. Immunol. (2015). In another embodiment, the bispecific molecule comprises an antibody which is fused to another non-Ig format, for example a T-cell receptor binding domain; an immunoglobulin superfamily domain; an agnathan variable lymphocyte receptor; a fibronectin domain (e.g. an Adnectin™); an antibody constant domain (e.g. a CH₃ domain, e.g., a CH₂ and/or CH₃ of an Fcab™) wherein the constant domain is not a functional CH₁ domain; an scFv; an (scFv)₂; an sc-diabody; an scFab; a centyrin and an epitope binding domain derived from a scaffold selected from CTLA-4 (Evibody™); a lipocalin domain; Protein A such as Z-domain of Protein A (e.g. an Affibody™ or SpA); an A-domain (e.g. an Avimer™ or Maxibody™); a heat shock protein (such as and epitope binding domain derived from GroEI and GroES); a transferrin domain (e.g. a trans-body); ankyrin repeat protein (e.g. a DARPin™); peptide aptamer; C-type lectin domain (e.g. Tetranectin™); human γ-crystallin or human ubiquitin (an affilin); a PDZ domain; scorpion toxin; and a kunitz type domain of a human protease inhibitor.

In one embodiment, the bispecific antibody is a mAb². A mAb² comprises a V_{H} and V_{L} domain from an intact antibody, fused to a modified constant region, which has been engineered to form an antigen-binding site, known as an “Fcab”. The technology behind the Fcab/mAb² format is described in more detail in WO2008/003103, and the description of the mAb² format is incorporated herein by reference.

In another embodiment, the bispecific antibody is a “dual binding antibody”. As used herein, the term “dual binding antibody” is a bispecific antibody wherein both antigen-binding domains are formed by a V_{H}/V_{L} pair, and includes FIT-Ig (see WO2015/103072, incorporated herein by reference), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv and scFv4-Ig.

The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the V_{H} (CDRH1, CDRH2, CDRH3), and three in the V_{L} (CDRL1, CDRL2, CDRL3). These regions of the heavy and light chains of an antibody confer antigen-binding specificity to the antibody. CDRs may be defined according to the Kabat system (see Kabat, E. A. et al., 1991, “Sequences of Proteins of Immunological Interest”, 5^{th} edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues. The term “specifically binds to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g. by a radioimmunoassay (RIA).

An antibody or a fragment thereof that specifically binds to a hBMP6 antigen may be cross-reactive with related antigens. Preferably, an antibody or a fragment thereof that specifically binds to a hBMP6 antigen does not cross-react with other antigens (but may optionally cross-react with BMP6 of a different species, e.g. rhesus, or murine). An antibody or a fragment thereof that specifically binds to a hBMP6 antigen can be identified, for example, by immunoassays, BIAcore™, or other techniques known to those of skill in the art. An antibody or a fragment thereof binds specifically to a BMP6 antigen when it binds to a hBMP6 antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times (such as more than 15 times, more than 20 times, more than 50 times or more than 100 times) background. See, e.g. Paul, ed., 1989, Fundamental Immunology Second Edition, Raven Press, New York at pages 332-336 for a discussion regarding antibody specificity.

The term “aliphatic amino acid” means that the amino acid R groups are nonpolar and hydrophobic. Hydrophobicity increases with increasing number of C atoms in the hydrocarbon chain. Glycine, Alanine, Valine, Leucine and Isoleucine are aliphatic amino acids.

The term “aromatic amino acid” means that the amino acid R groups contain an aromatic ring system. Phenylalanine, Tyrosine and Tryptophan are aromatic amino acids.

The term “hydroxyl-containing amino acid” means that the amino acid R groups contain a hydroxyl group and are hydrophilic. Serine, Cysteine, Threonine and Methionine are hydroxyl-containing amino acids.

The term “basic amino acid” means that the amino acid R groups are nitrogen containing and are basic at neutral pH. Histidine, Lysine and Arginine are basic amino acids.

The term “cyclic amino acid” means that the amino acid R groups have an aliphatic cyclic structure. Proline is the only cyclic aliphatic amino acid.

The term “acidic amino acid” means that the amino acid R groups are polar and are negatively charged at physiological pH. Aspartate and Glutamate are acidic amino acids.

The term “amide amino acid” means that the amino acid R groups contain an amide group. Asparagine and Glutamine are amide amino acids.

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g. the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

As used herein, a “buffer” refers to a chemical agent that is able to absorb a certain quantity of acid or base without undergoing a strong variation in pH.

As used herein, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

The term “chemotherapeutic agent” or “chemotherapy” refers to a therapeutic agent whose primary purpose is to destroy cancer cells, typically by interfering with the tumour cell's ability to grow or multiply. There are many different types of chemotherapeutic agents, with more than 50 approved chemotherapy drugs available. Chemotherapeutic drugs can be classified based on how they work. Alkylating drugs kill cancer cells by directly attacking DNA, the genetic material of the genes. Cyclophosphamide is an alkylating drug. Antimetabolites interfere with the production of DNA and keep cells from growing and multiplying. An example of an antimetabolite is 5-fluorouracil (5-FU). Anti-tumour antibiotics are made from natural substances such as fungi in the soil. They interfere with important cell functions, including production of DNA and cell proteins. Doxorubicin and bleomycin belong to this group of chemotherapy drugs. Plant alkaloids prevent cells from dividing normally. Vinblastine and vincristine are plant alkaloids obtained from the periwinkle plant. Steroid hormones slow the growth of some cancers that depend on hormones. For example, tamoxifen is used to treat breast cancers that depend on the hormone estrogen for growth. DNA damage response (DDR) inhibitors, such as PARP inhibitors, block DNA repair mechanisms following single or double stranded breaks.

Examples of chemotherapeutic agents include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19th Ed. (Mack Publishing Co. 1995), and in Goodman and Gilman's The Pharmacological Basis of Therapeutics, 7th Ed. (MacMillan Publishing Co. 1985). Another example of chemotherapeutic agents is the class of antibody-conjugated toxins, including, but not limited to pyrrolobenzodiazepiness, maytansanoids, calicheamicin, etc. Other suitable toxins and/or chemotherapeutic agents are known to those of skill in the art.

As used herein, the term “composition” is intended to encompass a product containing the specified ingredients (e.g. an antibody of the invention) in, optionally, the specified amounts, as well as any product which results, directly or indirectly, from combination of the specified ingredients in, optionally, the specified amounts.

As used herein the term “comprising” or “comprises” is used with reference to antibodies, fragments, uses, compositions, methods, and respective component(s) thereof, that are essential to the method or composition, yet open to the inclusion of unspecified elements, whether essential or not.

The term “consisting of” refers to antibodies, fragments, uses, compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

In the context of a polypeptide, the term “derivative” as used herein includes a polypeptide that comprises an amino acid sequence of a hBMP6 polypeptide, a fragment of a hBMP6 polypeptide, or an antibody or fragment that specifically binds to a hBMP6 polypeptide which has been altered by the introduction of amino acid residue substitutions, deletions or additions. The term “derivative” as used herein also includes a hBMP6 polypeptide, a fragment of a hBMP6 polypeptide, or an antibody that specifically binds to a hBMP6 polypeptide which has been chemically modified, e.g. by the covalent attachment of any type of molecule to the polypeptide. For example, but not by way of limitation, a hBMP6 polypeptide, a fragment of a hBMP6 polypeptide, or a hBMP6 antibody may be chemically modified, e.g. by glycosylation, acetylation, pegylation, phosphorylation, amidation, derivatization by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a hBMP6 polypeptide, a fragment of a hBMP6 polypeptide, or a hBMP6 antibody may be chemically modified by chemical modifications using techniques known to those of skill in the art, including, but not limited to specific chemical cleavage, acetylation, formulation, metabolic synthesis of tunicamycin, etc. Further, a derivative of a hBMP6 polypeptide, a fragment of a hBMP6 polypeptide, or a hBMP6 antibody may contain one or more non-classical amino acids. A polypeptide derivative possesses a similar or identical function as a hBMP6 polypeptide, a fragment of a hBMP6 polypeptide, or a hBMP6 antibody described herein.

The term “effector function” (or “effector-enabled”) as used herein refers to one or more of antibody dependant cell mediated cytotoxic activity (ADCC), complement-dependant cytotoxic activity (CDC) mediated responses, Fc-mediated phagocytosis or antibody dependant cellular phagocytosis (ADCP) and antibody recycling via the FcRn receptor.

An “effective amount” refers to an amount effective, at dosages and for periods of time necessary, to achieve the desired effect, including a therapeutic or prophylactic result. A “therapeutically effective amount” refers to the minimum concentration required to effect a measurable improvement or prevention of a particular disorder. A therapeutically effective amount herein may vary according to factors such as the disease state, age, sex, and weight of the patient, and the ability of the antibody to elicit a desired response in the individual. A therapeutically effective amount is also one in which toxic or detrimental effects of the antibody are outweighed by the therapeutically beneficial effects. A “prophylactically effective amount” refers to an amount effective, at the dosages and for periods of time necessary, to achieve the desired prophylactic result. In some embodiments, the effective amount of an antibody of the invention is from about 0.1 mg/kg (mg of antibody per kg weight of the subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some embodiments, “effective amount” as used herein also refers to the amount of an antibody of the invention to achieve a specified result (e.g. inhibition of a hBMP6 biological activity of a cell). The term “epitope” as used herein refers to a localized region on the surface of an antigen, such as hBMP6 polypeptide or hBMP6 polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three-dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a hBMP6 epitope is a three-dimensional surface feature of a hBMP6 polypeptide (e.g. in a trimeric form of a hBMP6 polypeptide). In other embodiments, a hBMP6 epitope is linear feature of a hBMP6 polypeptide (e.g. in a trimeric form or monomeric form of the hBMP6 polypeptide). Antibodies provided herein may specifically bind to an epitope of the monomeric (denatured) form of hBMP6, an epitope of the trimeric (native) form of hBMP6, or both the monomeric (denatured) form and the trimeric (native) form of hBMP6. In specific embodiments, the antibodies provided herein specifically bind to an epitope of the trimeric form of hBMP6 but do not specifically bind the monomeric form of hBMP6.

The term “excipients” as used herein refers to inert substances which are commonly used as a diluent, vehicle, preservatives, binders, or stabilizing agent for drugs and includes, but not limited to, proteins (e.g. serum albumin, etc.), amino acids (e.g. aspartic acid, glutamic acid, lysine, arginine, glycine, histidine, etc.), fatty acids and phospholipids (e.g. alkyl sulfonates, caprylate, etc.), surfactants (e.g. SDS, polysorbate, nonionic surfactant, etc.), saccharides (e.g. sucrose, maltose, trehalose, etc.) and polyols (e.g. mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, BMP6 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a hBMP6 polypeptide or an antibody that specifically binds to a hBMP6 polypeptide. In a specific embodiment, a fragment of a hBMP6 polypeptide or an antibody that specifically binds to a hBMP6 antigen retains at least 1, at least 2, or at least 3 functions of the polypeptide or antibody.

The term “free” can refer to a polypeptide, for example, BMP6 or fragments and variants thereof, that is combined with a buffer, wherein the polypeptide is not associated with a cell surface or cell membrane. As such, the term “free” can refer to a polypeptide that is capable of surface expression (i.e. includes one or more transmembrane domains or membrane association domains), but that is not, in its present state, expressed on the surface of a cell or bound to a protein that is expressed on the surface of a cell. A free polypeptide can also refer to a free recombinant or native or unbound polypeptide. In the context of phage display, a free antigen can be selected in solution (referred to herein as a “soluble selection”) or adsorbed to a surface, for example, adsorbed to the surface of a 96-well plate (referred to herein as “biopanning selection”).

The term “fusion protein” as used herein refers to a polypeptide that comprises an amino acid sequence of an antibody and an amino acid sequence of a heterologous polypeptide or protein (i.e. a polypeptide or protein not normally a part of the antibody (e.g. a non-anti-BMP6 antigen antibody)). The term “fusion” when used in relation to BMP6 or to an anti-BMP6 antibody refers to the joining of a peptide or polypeptide, or fragment, variant and/or derivative thereof, with a heterologous peptide or polypeptide. Preferably, the fusion protein retains the biological activity of the BMP6 or anti-BMP6 antibody. In certain embodiments, the fusion protein comprises a BMP6 antibody VH domain, VL domain, VH CDR (one, two or three VH CDRs), and/or VL CDR (one, two or three VL CDRs), wherein the fusion protein specifically binds to a BMP6 epitope.

The term “heavy chain” when used with reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG2, IgG3 and IgG4. Preferably the heavy chain is a human heavy chain. In the human population, multiple heavy chain constant region alleles, of each immunoglobulin or immunoglobulin subclass, exist. The nucleotide and amino acid sequences of these allelic variants are accessible on publicly available databases such as IMGT, ENSEMBL Swiss-Prot and Uniprot. Allelic variants may also be identified in various genome sequencing projects. In one embodiment, the antibodies and antibody fragments disclosed herein comprise a heavy chain encoded by a IgG1 constant region allele, which includes, but is not limited to, human IGHG1*01 (Seq ID Nos: 340, 341 & 537), IGHG1*02 (Seq ID Nos: 340, 341 &537), IGHG1*03 (Seq ID Nos: 523 & 524), IGHG1*04 (Seq ID Nos: 525 & 526) and IGHG1*05 (Seq ID Nos: 340, 341 & 537). In one embodiment, the antibodies and antibody fragments disclosed herein comprise a protein encoded by a IgG2 constant region allele, which includes, but is not limited to, human IGHG2*01 (Seq ID Nos: 527 & 528), IGHG2*02 (Seq ID Nos: 529 & 530), IGHG2*03 (Seq ID Nos: 527 & 528), IGHG2*04 (Seq ID Nos: 531 & 532), IGHG2*05 (Seq ID Nos: 527 & 528) and IGHG2*06 (Seq ID Nos: 533 & 534). In one embodiment, the antibodies or antibody fragments disclosed herein comprise a protein encoded by a IgG3 constant region allele, which includes but is not limited to human IGHG3*01, IGHG3*02, IGHG3*03, IGHG3*04, IGHG3*05, IGHG3*06, IGHG3*07, IGHG3*08, IGHG3*09, IGHG3*10, IGHG3*11, IGHG3*12, IGHG3*13, IGHG3*14, IGHG3*15, IGHG3*16, IGHG3*17, IGHG3*18 and IGHG3*19. In one embodiment, the antibodies or antibody fragments disclosed herein comprise a protein encoded by a IgG4 constant region allele, which includes but is not limited to human IGHG4*01 (see, eg, the sequence table herein), IGHG4*02 (see, eg, the sequence table herein), IGHG4*03 (see, eg, the sequence table herein) and IGHG4*04 (see, eg, the sequence table herein). In another example, the heavy chain is a disabled IgG isotype, e.g. a disabled IgG4. In certain embodiments, the antibodies of the invention comprise a human gamma 4 constant region. In another embodiment, the heavy chain constant region does not bind Fc-γ receptors, and e.g. comprises a Leu235Glu mutation. In another embodiment, the heavy chain constant region comprises a Ser228Pro mutation to increase stability. In another embodiment, the heavy chain constant region is IgG4-PE (see, eg, the sequence table herein). In another embodiment, the antibodies and antibody fragments disclosed herein comprise a heavy chain constant region encoded by a murine IgG1 constant region allele, which includes but is not limited to mouse IGHG1*01 or IGHG1*02. In one embodiment, the antibodies and antibody fragments disclosed herein comprise a heavy chain constant region encoded by a murine IgG2 constant region allele, which includes, but is not limited to, mouse IGHG2A*01, IGHG2A*02, IGHG2B*01, IGHG2B*02, IGHG2C*01, IGHG2C*02 or IGHG2C*03. In one embodiment, the antibodies or antibody fragments disclosed herein comprise a protein encoded by a murine IgG3 constant region allele, which includes but is not limited to mouse IGHG3*01.

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “in combination” in the context of the administration of other therapies refers to the use of more than one therapy. The use of the term “in combination” does not restrict the order in which therapies are administered to a subject with a disease. A first therapy can be administered before (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a BMP6-mediated disease. Any additional therapy can be administered in any order with the other additional therapies. In certain embodiments, the antibodies of the invention can be administered in combination with one or more therapies (e.g. therapies that are not the antibodies of the invention that are currently administered to prevent, treat, manage, and/or ameliorate a BMP6-mediated disease. Non-limiting examples of therapies that can be administered in combination with an antibody of the invention include analgesic agents, anaesthetic agents, antibiotics, or immunomodulatory agents or any other agent listed in the U.S. Pharmacopoeia and/or Physician's Desk Reference.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some embodiments, an injection device can be an intravenous device or IV device, which is a type of injection device used when the target tissue is the blood within the circulatory system, e.g. the blood in a vein. A common, but non-limiting example of an injection device is a needle and syringe. As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody or protein is one that has been identified, separated and/or recovered from a component of its production environment (e.g. natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e. culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly, such preparations of the antibody have less than about 30%, 20%, 10%, 5% (by dry weight) of chemical precursors or compounds other than the antibody of interest. In a preferred embodiment, antibodies of the invention are isolated or purified.

The terms “Kabat numbering,” and like terms are recognized in the art and refer to a system of numbering amino acid residues which are more variable (i.e. hypervariable) than other amino acid residues in the heavy chain variable regions of an antibody, or an antigen binding portion thereof (Kabat et al., (1971) Ann. NY Acad. Sci., 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

“Label” or “labelled” as used herein refers to the addition of a detectable moiety to a polypeptide, for example, a radiolabel, fluorescent label, enzymatic label, chemiluminescent label or a biotinyl group or gold. Radioisotopes or radionuclides may include ³H, ¹⁴C, ¹⁵N, ³⁵S, ⁹⁰Y, ⁹⁹Tc, ¹¹⁵ In, ¹²⁵I, ¹³¹I, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes (e.g. cyanine dyes, e.g. Cy5™, Cy5.5™. or Cy7™); additional fluorescent labels or fluorescers include, such as fluorescein and its derivatives, fluorochrome, GFP (GFP for “Green Fluorescent Protein”), other fluorescent proteins (e.g. mCherry, mTomato), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “light chain” when used in reference to an antibody refers to the immunoglobulin light chains, of which there are two types in mammals, lambda (λ) and kappa (κ). Preferably, the light chain is a human light chain. Preferably the light chain constant region is a human constant region. In the human population, multiple light chain constant region alleles exist. The nucleotide and amino acid sequences of these allelic variants are accessible on publicly available databases such as IMGT, ENSEMBL, Swiss-Prot and Uniprot. In one embodiment, the antibodies or antibody fragments disclosed herein comprise a protein encoded by a human κ constant region allele, which includes, but is not limited to, IGKC*01 (see, eg, the sequence table herein), IGKC*02 (see, eg, the sequence table herein), IGKC*03 (see, eg, the sequence table herein), IGKC*04 (see, eg, the sequence table herein) and IGKC*05 (see, eg, the sequence table herein). In one embodiment, the antibodies or antibody fragments disclosed herein comprise a protein encoded by a human A constant region allele, which includes but is not limited to IGLC1*01 (see, eg, the sequence table herein), IGLC1*02 (see, eg, the sequence table herein), IGLC2*01 (see, eg, the sequence table herein), IGLC2*02 (see, eg, the sequence table herein), IGLC2*03 (see, eg, the sequence table herein), IGLC3*01 (see, eg, the sequence table herein), IGLC3*02 (see, eg, the sequence table herein), IGLC3*03 (see, eg, the sequence table herein), IGLC3*04 (see, eg, the sequence table herein), IGLC6*01 (see, eg, the sequence table herein), IGLC7*01 (see, eg, the sequence table herein), IGLC7*02 (see, eg, the sequence table herein), IGLC7*03 (see, eg, the sequence table herein). In another embodiment, the antibodies and antibody fragments disclosed herein comprise a light chain constant region encoded by a mouse k constant region allele, which includes, but is not limited to, IGKC*01, IGKC*03 or IGKC*03. In another embodiment, the antibodies and antibody fragments disclosed herein comprise a light chain constant region encoded by a mouse A constant region allele, which includes, but is not limited to, IGLC1*01, IGLC2*01 or IGLC3*01.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEG ALIGN™ (DNASTAR) software. In one embodiment, the % homology is about 70%. In one embodiment, the % homology is about 75%. In one embodiment, the % homology is about 80%. In one embodiment, the % homology is about 85%. In one embodiment, the % homology is about 90%. In one embodiment, the % homology is about 92%. In one embodiment, the % homology is about 95%. In one embodiment, the % homology is about 97%. In one embodiment, the % homology is about 98%. In one embodiment, the % homology is about 99%. In one embodiment, the % homology is 100%.

The term “naturally occurring” or “native” when used in connection with biological materials such as nucleic acid molecules, polypeptides, host cells, and the like, refers to those which are found in nature and not manipulated by a human being.

As used herein, “packaging” refers to how the components are organized and/or restrained into a unit fit for distribution and/or use. Packaging can include, e.g. boxes, bags, syringes, ampoules, vials, tubes, clamshell packaging, barriers and/or containers to maintain sterility, labelling, etc. The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

As used herein, the terms “prevent”, “preventing”, and “prevention” refer to the total or partial inhibition of the development, recurrence, onset or spread of a hBMP6-mediated disease and/or symptom related thereto, resulting from the administration of a therapy or combination of therapies provided herein (e.g. a combination of prophylactic or therapeutic agents, such as an antibody of the invention).

The term “soluble” refers to a polypeptide, such as BMP6 and variants or fragments thereof, that is lacking one or more transmembrane or cytoplasmic domains found in the native or membrane-associated form. In one embodiment, the “soluble” form of BMP6 lacks both the transmembrane domain and the cytoplasmic domain.

The term “subject” or “patient” refers to any animal, including, but not limited to, mammals. As used herein, the term “mammal” refers to any vertebrate animal that suckle their young and either give birth to living young (eutharian or placental mammals) or are egg-laying (metatharian or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in the treatment, management or amelioration of a BMP6-mediated disease and/or a symptom related thereto. In certain embodiments, the term “therapeutic agent” refers to an antibody of the invention. In certain other embodiments, the term “therapeutic agent” refers to an agent other than an antibody of the invention. Preferably, a therapeutic agent is an agent which is known to be useful for, or has been or is currently being used for the treatment, management or amelioration of a BMP6-mediated disease or one or more symptoms related thereto. In specific embodiments, the therapeutic agent is a fully human anti-BMP6 antibody, such as a fully human anti-BMP6 monoclonal antibody.

As used herein, the term “therapy” refers to any protocol, method and/or agent that can be used in the prevention, management, treatment and/or amelioration of a BMP6-mediated disease (e.g. cancer). In certain embodiments, the terms “therapies” and “therapy” refer to a biological therapy, supportive therapy, and/or other therapies useful in the prevention, management, treatment and/or amelioration of a BMP6-mediated disease known to one of skill in the art such as medical personnel. The terms “treat”, “treatment” and “treating” refer to the reduction or amelioration of the progression, severity, and/or duration of a hBMP6-mediated disease (e.g. cancer) resulting from the administration of one or more therapies (including, but not limited to, the administration of one or more prophylactic or therapeutic agents, such as an antibody of the invention). In specific embodiments, such terms refer to the reduction or inhibition of the binding of hBMP6 to a BMP receptor or HJV, and/or the inhibition or reduction of one or more symptoms associated with a BMP6-mediated disease, such as anaemia.

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs of the BMP6 and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5th ed. (“Kabat et al.”). In preferred embodiments, the variable region is a human variable region.

Definitions of common terms in cell biology and molecular biology can be found in “The Merck Manual of Diagnosis and Therapy”, 19^{th} Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10:0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

### BMP6 & Iron

Since iron is fundamental to all forms of life and has to be sourced from the environment, the availability and usage in the body is tightly controlled. A key regulator of iron homeostasis is a 25 amino acid peptide hormone called hepcidin. Hepcidin is produced by the liver and causes the main iron uptake and storage compartments, the duodenal enterocytes and macrophages, to retain iron by means of controlling expression of the iron transporter molecule ferroportin. Hepcidin itself is regulated by iron levels through a homeostatic control mechanism, following activation of the immune system during infection and/or inflammation as well as by erythropoiesis. Importantly, hepcidin levels are elevated in chronic inflammatory situations, infections and also certain cancers. Elevated hepcidin levels sequester iron in enterocytes, macrophages and hepatocytes thereby suppressing haemoglobin synthesis and erythropoiesis. This leads to anaemia despite the fact that iron storage levels are normal. Hepcidin gene expression is controlled by a soluble factor called BMP6 (bone morphogenetic protein 6). BMP6 is considered the master regulator, since in the absence of BMP6, cytokines alone (or other BMPs) are not able to overcome the deficit of a BMP6 signal. The inventors thus focused on BMP6 is a key drug target for controlling aberrant dysregulation of iron homeostasis in anaemia, eg, in anaemia of chronic disease (ACD).

BMP6 is a highly conserved soluble protein factor that is considered the “master” regulator of hepcidin production in mice and humans. Hence, administration of BMP6 to mice increases hepcidin levels and decreases blood and serum iron, while inhibitors of BMP6 do the opposite. In addition, knock-out of the mouse BMP6 gene or human mutations within the BMP6 pathway support a central role for BMP6 in controlling hepcidin and blood and serum iron levels. Furthermore, pre-clinical and clinical validation for targeting BMP6 comes from increasing available iron levels by administering an anti-BMP6 antibody to rodents or cynomolgus monkeys or BMP6 neutralization using HJV-Fc (FMX-8, Ferrumax Inc) in a Phase I study, respectively. Reference is made to Andriopoulos Jr. B, Corradini E, Xia Y, Faasse S A, Chen S, Grgurevic L, Knutson M D, Pietrangelo A, Vukicevic S, Lin H Y and Babitt. 2009. BMP-6 is a key endogenous regulator of hepcidin expression and iron metabolism. Nat. Genet. 41 (4), 482-487; WO2016098079 and U.S. Pat. No. 8,980,582.

### Anti-BMP6 Antibodies & Fragments

The invention provides various anti-BMP6 antibodies and fragments (such as Fab or scFv fragments), uses, methods and combinations (eg, with ESA). Examples are set out in the following numbered Clauses.
1. An antibody or fragment comprising a binding site which specifically binds to Bone Morphogenetic Protein 6 (BMP6), wherein the binding site comprises a VH domain that is encoded by a nucleotide sequence that is derived from the recombination of a human VH gene segment, DH gene segment and JH gene segment, wherein the VH gene segment is selected from IGHV3-11 and IGHV1-3.

For example, the VH gene segment is IGHV3-11 and the DH gene segment and JH gene segments are human gene segments. For example, the VH gene segment is IGHV1-3 and the DH gene segment and JH gene segments are human gene segments. Optionally, the VH segment is a human IGHV3-11*01 gene segment. Alternatively, optionally the VH segment is a human IGHV1-3*01 gene segment.

In an example, specific binding is with a KD, K_{off} and/or Kₒₙ as described further below. In an example, specific binding is with a KD from 1 pM to 5 nM.

The skilled person is familiar with databases and other sources for human and other species of antibody gene segments. For example, the IMGT database (world wide web.IMGT.org) is a suitable source, eg, the version as at 1 Sep. 2018.

Reference is made to Tables 7 and 8, showing antibodies that are based on IGHV1-3. Surprisingly, this human VH gene segment produces anti-BMP6 antibodies which have desirable anti-BMP6 properties, such as those described in, eg, in the Examples.

Reference is made to Table 9, showing antibodies that are based on IGHV3-11. Surprisingly, this human VH gene segment produces anti-BMP6 antibodies which have desirable anti-BMP6 properties, such as those described in, eg, in the Examples. Thus, for example, the antibody or fragment comprises a CDRH3 sequence selected from SEQ ID NO: 110, 113, 290, 293, 308, 311, 272 and 275. For example, the antibody or fragment comprises a CDRL3 sequence selected from SEQ ID NO: 119, 122, 299, 302, 317, 320, 281 and 284. For example, the antibody or fragment comprises a CDRH3 sequence selected from SEQ ID NO: 110, 113, 290, 293, 308, 311, 272 and 275 and respectively a CDRL3 sequence selected from SEQ ID NO: 119, 122, 299, 302, 317, 320, 281 and 284.

For example, the antibody or fragment comprises a CDRH3 sequence selected from SEQ ID NO: 110 and 113; and a CDRL3 sequence selected from SEQ ID NO: 119 and 122. For example, the antibody or fragment comprises an anti-BMP6 binding site, wherein the binding site comprises a VH domain comprising SEQ ID NO: 110 paired with a VL domain comprising SEQ ID NO: 119. For example, the antibody or fragment comprises an anti-BMP6 binding site, wherein the binding site comprises a VH domain comprising SEQ ID NO: 113 paired with a VL domain comprising SEQ ID NO: 122.

For example, the antibody or fragment comprises a CDRH3 sequence of an antibody selected from CL-58838, CL-58835, CL-58756 and CL-58722 and optionally a CDRL3 of said selected antibody. For example, the antibody or fragment comprises a CDRH1 and CDRH3 sequence of an antibody selected from CL-58838, CL-58835, CL-58756 and CL-58722 and optionally a CDRL2 of said selected antibody. For example, the antibody or fragment comprises a CDRH1 and CDRH2 sequence of an antibody selected from CL-58838, CL-58835, CL-58756 and CL-58722. For example, the antibody or fragment comprises a CDRH2 and CDRH3 sequence of an antibody selected from CL-58838, CL-58835, CL-58756 and CL-58722. For example, the antibody or fragment comprises an anti-BMP6 binding site, wherein the binding site comprises a VH domain comprising the CDRH3 sequence of CL-58838 paired with a VL domain of CL-58838.

For example, the antibody or fragment comprises an anti-BMP6 binding site, wherein the binding site comprises a VH domain comprising SEQ ID NO: 114, 294, 312 or 276 optionally paired with a VL domain comprising respectively SEQ ID NO: 123, 303, 321 or 285. For example, the antibody or fragment comprises an anti-BMP6 binding site, wherein the binding site comprises a VH domain comprising SEQ ID NO: 114 paired with a VL domain comprising SEQ ID NO: 123.

For example, the antibody or fragment comprises an anti-BMP6 binding site, wherein the binding site comprises a VH domain of an antibody selected from CL-58838, CL-58835, CL-58756 and CL-58722, optionally paired with a VL domain of the selected antibody. For example, the antibody or fragment comprises an anti-BMP6 binding site, wherein the binding site comprises a VH domain of CL-58838 paired with a VL domain of CL-58838.
2. The antibody or fragment according to Clause 1, wherein the DH gene segment is a human gene segment selected from IGHD3-10, IGHD6-19, IGHD7-27, IGHD4-23, IGHD5-18, IGHD3-22 and IGHD3-16.

Optionally, the DH gene segment is selected from IGHD3-10*01, IGHD6-19*01, IGHD7-27*02, IGHD4-23*01, IGHD5-18*01, IGHD3-22*01 and IGHD3-16*02.
3. The antibody or fragment according to Clause 1 or 2, wherein the JH gene segment is a human gene segment selected from IGHJ3, IGHJ4 and IGHJ5.

Optionally, the JH gene segment is selected from IGHJ3*02, IGHJ4*02 and IGHJ5*02.
4. An antibody or fragment which specifically binds to Bone Morphogenetic Protein 6 (BMP6) and comprises the CDRH3 sequence of an anti-BMP6 antibody according to any preceding Clause.
5. An antibody or fragment which specifically binds to Bone Morphogenetic Protein 6 (BMP6) and comprises a VH domain which comprises the CDRH3 sequence of any anti-BMP6 antibody disclosed herein (eg, any antibody selected from an antibody or fragment listed in any of Tables 4 to 11 herein), or said CDRH3 sequence comprising 3, 2 or 1 amino acid substitution(s).
6. An antibody or fragment (optionally according to any preceding Clause) which specifically binds to Bone Morphogenetic Protein 6 (BMP6) and comprises a VH domain which comprises a CDRH3 sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said sequence comprising 3, 2 or 1 amino acid substitution(s).

Optionally, the VH domain comprises a CDRH3 sequence selected from SEQ ID NO: 110 or 113, or said selected sequence comprising 3, 2 or 1 amino acid substitution(s).
7. The antibody or fragment according to Clause 6, wherein the VH domain comprises (i) a CDRH3 sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said CDRH3 sequence comprising 3, 2 or 1 amino acid substitution(s); and (ii) a CDRH1 sequence of said selected antibody; or said CDRH1 sequence comprising 3, 2 or 1 amino acid substitution(s).

Optionally, the VH domain of the antibody or fragment comprises (a) the CDRH3 sequence of SEQ ID NO: 110 or 113; or said CDRH3 sequence comprising 3, 2 or 1 amino acid substitution(s); and (b) the CDRH1 sequence of SEQ ID NO: 108 or 111, or said CDRH1 sequence comprising 3, 2 or 1 amino acid substitution(s).
8. The antibody or fragment according to Clause 6 or 7, wherein the VH domain comprises (iii) a CDRH3 sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said CDRH3 sequence comprising 3, 2 or 1 amino acid substitution(s); and (iv) a CDRH2 sequence of said selected antibody; or said CDRH2 sequence comprising 3, 2 or 1 amino acid substitution(s).

Optionally, the VH domain of the antibody or fragment comprises (c) the CDRH3 sequence of SEQ ID NO: 110 or 113; or said CDRH3 sequence comprising 3, 2 or 1 amino acid substitution(s); and (d) the CDRH2 sequence of SEQ ID NO: 109 or 112, or said CDRH2 sequence comprising 3, 2 or 1 amino acid substitution(s).

Optionally, the VH domain of the antibody or fragment comprises (e) the CDRH3 sequence of SEQ ID NO: 110 or 113; or said CDRH3 sequence comprising 3, 2 or 1 amino acid substitution(s); (f) the CDRH1 sequence of SEQ ID NO: 108 or 111, or said CDRH1 sequence comprising 3, 2 or 1 amino acid substitution(s); and (g) the CDRH2 sequence of SEQ ID NO: 109 or 112, or said CDRH2 sequence comprising 3, 2 or 1 amino acid substitution(s).
9. An antibody or fragment (optionally according to any preceding Clause) comprising a binding site which specifically binds to Bone Morphogenetic Protein 6 (BMP6), wherein the binding site comprises a VH domain that comprises the amino acid sequence of a VH domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

For example, the identity is at least 85%. For example, the identity is at least 90%. For example, the identity is at least 95%.

Optionally the VH domain of the antibody or fragment of comprises the amino acid sequence of SEQ ID NO: 114, or a heavy chain variable domain amino acid sequence that is at least 70, 80, 85, 90, 95, 96, 97, 98 or 99% identical to SEQ ID NO: 114. For example, the identity is at least 85%. For example, the identity is at least 90%. For example, the identity is at least 95%.
10. The antibody or fragment according to any preceding Clause comprising first and second copies of said VH domain.

In an example, the antibody or fragment comprises a binding site comprising a VH domain of the invention paired with a VL domain of the invention, wherein the binding site is capable of specifically binding to BMP6 (eg, mature BMP6, eg human and/or cynomolgus monkey BMP6). For example, the antibody or fragment comprise two of such binding sites.
11. An antibody or fragment (optionally according to any preceding Clause) comprising a binding site which specifically binds to Bone Morphogenetic Protein 6 (BMP6), wherein the binding site comprises a VL domain that is encoded by a nucleotide sequence that is derived from the recombination of a human VL gene segment and JL gene segment, wherein the VL gene segment is selected from IGKV3-20, IGKV1-5 and IGKV3-15.

For example, the VL gene segment is IGKV3-20, eg, IGKV3-20*01. For example, the VL gene segment is IGKV1-5, eg, IGKV1-5*03. For example, the VL gene segment is IGKV3-15, eg, IGKV3-15*01.
12. The antibody or fragment according to Clause 11, wherein the VL is a Vκ and the JL gene segment is a human gene segment selected from IGKJ1 and IGKJ3.

Optionally, the JL gene segment is selected from IGKJ1*01 and IGKJ3*01.
13. An antibody or fragment which specifically binds to Bone Morphogenetic Protein 6 (BMP6) and comprises the CDRL3 sequence of an anti-BMP6 antibody according to Clause 11 or 12.
14. An antibody or fragment (optionally according to any preceding Clause) which specifically binds to Bone Morphogenetic Protein 6 (BMP6) and comprises a VL domain which comprises the CDRL3 sequence of any anti-BMP6 antibody disclosed herein (eg, any antibody selected from an antibody or fragment listed in any of Tables 4 to 11 herein) or said selected CDRL3 sequence comprising 3, 2 or 1 amino acid substitution(s).
15. The antibody or fragment of Clause 14, comprising a VH domain which comprises the CDRH3 sequence of said selected antibody.
16. An antibody or fragment (optionally according to any preceding Clause) which specifically binds to Bone Morphogenetic Protein 6 (BMP6) and comprises a VL domain which comprises a CDRL3 sequence selected from SEQ ID NO: 119 or 122, or said selected CDRL3 sequence comprising 3, 2 or 1 amino acid substitution(s).
17. An antibody or fragment (optionally according to any preceding Clause) which specifically binds to Bone Morphogenetic Protein 6 (BMP6) and comprises a VL domain which comprises a CDRL3 (and optionally a CDRH3) sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said sequence(s) each comprising 3, 2 or 1 amino acid substitution(s).

Optionally, the VL domain comprises a CDRL3 sequence selected from SEQ ID NO: 119 or 122, or said selected sequence comprising 3, 2 or 1 amino acid substitution(s) and/or optionally, the VH domain comprises a CDRH3 sequence selected from SEQ ID NO: 119 or 122, or said selected sequence comprising 3, 2 or 1 amino acid substitution(s).
18. The antibody or fragment according to Clause 17, wherein the VL domain comprises (i) a CDRL3 sequence (and optionally a CDRH3) of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said CDR3 sequence(s) each comprising 3, 2 or 1 amino acid substitution(s); and (ii) a CDRL1 (and optionally a CDRH1) sequence of said selected antibody; or said CDR1 sequence(s) each comprising 3, 2 or 1 amino acid substitution(s).

Optionally, the VL domain of the antibody or fragment comprises (a) the CDRL3 sequence of SEQ ID NO: 119 or 122; or said CDRL3 sequence comprising 3, 2 or 1 amino acid substitution(s); and (b) the CDRL1 sequence of SEQ ID NO: 117 or 120, or said CDRL1 sequence comprising 3, 2 or 1 amino acid substitution(s).
19. The antibody or fragment according to Clause 17 or 18, wherein the VL domain comprises (iii) a CDRL3 (and optionally a CDRH3) sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or said CDR3 sequence(s) each comprising 3, 2 or 1 amino acid substitution(s); and (iv) a CDRL2 (and optionally a CDRH2) sequence of said selected antibody; or said CDR2 sequence(s) each comprising 3, 2 or 1 amino acid substitution(s).

Optionally, the VL domain of the antibody or fragment comprises (c) the CDRL3 sequence of SEQ ID NO: 119 or 122; or said CDRL3 sequence comprising 3, 2 or 1 amino acid substitution(s); and (d) the CDRL2 sequence of GAF or SEQ ID NO: 121, or said CDRL2 sequence comprising 3, 2 or 1 amino acid substitution(s).

Optionally, the VL domain of the antibody or fragment comprises (e) the CDRL3 sequence of SEQ ID NO: 119 or 122; or said CDRL3 sequence comprising 3, 2 or 1 amino acid substitution(s); (f) the CDRL1 sequence of SEQ ID NO: 117 or 120, or said CDRL1 sequence comprising 3, 2 or 1 amino acid substitution(s); and (g) the CDRL2 sequence of GAF or SEQ ID NO: 121, or said CDRL2 sequence comprising 3, 2 or 1 amino acid substitution(s).
20. An antibody or fragment (optionally according to any preceding Clause) comprising a binding site which specifically binds to Bone Morphogenetic Protein 6 (BMP6), wherein the binding site comprises a VL domain that comprises the amino acid sequence of a VL domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

For example, the identity is at least 85%. For example, the identity is at least 90%. For example, the identity is at least 95%.

Optionally the VL domain of the antibody or fragment of comprises the amino acid sequence of SEQ ID NO: 123, or a heavy chain variable domain amino acid sequence that is at least 70, 80, 85, 90, 95, 96, 97, 98 or 99% identical to SEQ ID NO: 123. For example, the identity is at least 85%. For example, the identity is at least 90%. For example, the identity is at least 95%.
21. The antibody or fragment according to any preceding Clause comprising first and second copies of said VL domain.

In an example, the antibody or fragment comprises a binding site comprising a VL domain of the invention paired with a VH domain, wherein the binding site is capable of specifically binding to BMP6 (eg, mature BMP6, eg human and/or cynomolgus monkey BMP6). For example, the antibody or fragment comprise two of such binding sites.
22. An antibody or fragment (optionally according to any preceding Clause) which specifically binds to Bone Morphogenetic Protein 6 (BMP6) and comprises the heavy chain amino acid sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

For example, the identity is at least 85%. For example, the identity is at least 90%. For example, the identity is at least 95%.
23. An antibody or fragment (optionally according to any preceding Clause) which specifically binds to Bone Morphogenetic Protein 6 (BMP6) and comprises the light chain amino acid sequence of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

For example, the identity is at least 85%. For example, the identity is at least 90%. For example, the identity is at least 95%.
24. The antibody or fragment of Clause 23, comprising the light chain amino acid sequence of said selected antibody; or an amino acid that is at least 70, 80, 85, 90, 95, 96, 97, 98 or 99% identical thereto.

For example, the identity is at least 85%. For example, the identity is at least 90%. For example, the identity is at least 95%.
25. An antibody or fragment (optionally according to any preceding Clause) which specifically binds to a human BMP6 epitope that is identical to an epitope to which the antibody of any preceding Clause binds.
26. The antibody or fragment according to Clause 25, wherein the epitope is identified by unrelated amino acid scanning, or by X-ray crystallography.

Contact amino acid residues involved in the interaction of antibody and antigen may be determined by various known methods to those skilled in the art.

In one embodiment, sequential replacement of the amino acids of the antigen sequence (using standard molecular biology techniques to mutate the DNA of the coding sequence of the antigen), in this case BMP6 with Alanine (a.k.a Alanine scan), or another unrelated amino acid, may provide residues whose mutation would reduce or ablate the ability of the antibody to recognise the antigen in question. Binding may be assessed using standard techniques, such as, but not limited to, SPR, HTRF, ELISA (which are described elsewhere herein). Other substitutions could be made to enhance the disruption of binding such as changing the charge on the side chain of antigen sequence amino acids (e.g. Lysine change to glutamic acid), switching polar and non-polar residues (e.g. Serine change to leucine). The alanine scan or other amino substitution method may be carried out either with recombinant soluble antigen, or where the target is a cell membrane target, directly on cells using transient or stable expression of the mutated versions.

In one embodiment, protein crystallography may be used to determine contact residues between antibody and antigen (i.e. to determine the epitope to which the antibody binds), crystallography allows the direct visualisation of contact residues involved in the antibody-antigen interaction. As well as standard X-ray crystallography, cryo-electro microscopy has been used to determine contact residues between antibodies and HIV capsid protein (see Lee, Jeong Hyun, et al. “Antibodies to a conformational epitope on gp41 neutralize HIV-1 by destabilizing the Env spike.”, Nature communications, 6, (2015)).

In one embodiment, if the antibody recognises a linear epitope, short peptides based on the antigen sequence can be produced and binding of the antibody to these peptides can be assessed using standard techniques, such as, but not limited to, SPR, HTRF, ELISA (which are described elsewhere herein). Further investigation of the epitope could be provided by performing an Alanine scan on any peptides that show binding. Alternative to linear peptides, conformational scans could be carried out using Pepscan technology (world wide web.pepscan.com) using their chemical linkage of peptides onto scaffolds, which has been used to determine discontinuous epitopes on CD20 targeting antibodies (Niederfellner, Gerhard, et al. “Epitope characterization and crystal structure of GA101 provide insights into the molecular basis for type I/II distinction of CD20 antibodies.”, Blood, 118.2, (2011), 358-367).

In one embodiment, limited proteolytic digestion and mass spectrophotometry can be used to identify binding epitopes. The antibody-antigen complex is digested by a protease, such as, but not limited to, trypsin. The digested complex peptides are compared to antibody-alone and antigen-alone digestion mass spectrophotometry to determine if a particular epitope is protected by the complexation. Further work involving amino acid substitution, competition binding, may then be employed to narrow down to individual amino acid residues involved in the interaction (see, for example, Suckau, Detlev, et al. “Molecular epitope identification by limited proteolysis of an immobilized antigen-antibody complex and mass spectrometric peptide mapping.”, Proceedings of the National Academy of Sciences, 87.24, (1990), 9848-9852).

Thus, in one embodiment, the contact residues of the epitope are identified with an unrelated amino acid scan (e.g. alanine scan). In another embodiment, an unrelated amino acid scan (e.g. alanine scan) is carried out using a technique selected from SPR, HTRF, ELISA, X-ray crystallography, cryo-electro microscopy and a combination of limited proteolytic digestion and mass spectrometry. In one embodiment, the unrelated amino acid scan (e.g. alanine scan) is carried out using HTRF. In one embodiment, the unrelated amino acid scan (e.g. alanine scan) is carried out using ELISA. When the alanine scan is carried out with either ELISA or HTRF, an amino acid residue is identified as contributing to the epitope if the reduction in signal is at least 25%. In one embodiment, the reduction in signal is at least 30%. In one embodiment, the reduction in signal is at least 35%. In one embodiment, the reduction in signal is at least 40%. In one embodiment, the reduction in signal is at least 45%. In one embodiment, the reduction in signal is at least 50%. In one embodiment, the reduction in signal is at least 55%. In one embodiment, the reduction in signal is at least 60%. In one embodiment, the reduction in signal is at least 70%. In one embodiment, the reduction in signal is at least 75%. In one embodiment, the reduction in signal is at least 80%. In one embodiment, the reduction in signal is at least 85%. In one embodiment, the reduction in signal is at least 90%. When the alanine scan is carried out with SPR, an amino acid residue is identified as contributing to the epitope if there is at least a 10-fold reduction in affinity. In one embodiment, the reduction in affinity is at least 15-fold. In one embodiment, the reduction in affinity is at least 20-fold. In one embodiment, the reduction in affinity is at least 30-fold. In one embodiment, the reduction in affinity is at least 40-fold. In one embodiment, the reduction in affinity is at least 50-fold. In one embodiment, the reduction in affinity is at least 100-fold.

In one embodiment, the contact residues of the epitope are identified by X-ray crystallography. In one embodiment, the contact residues of the epitope are identified by cryo-electro microscopy. In one embodiment, the contact residues of the epitope are identified by a combination of limited proteolytic digestion and mass spectrometry.
27. The antibody or fragment according to Clause 26, wherein the contact residues of the epitope are defined by a reduction in affinity of at least 10-fold in an unrelated amino acid scan, e.g. an alanine scan as determined by SPR.

In one embodiment, the reduction in affinity is at least 15-fold. In one embodiment, the reduction in affinity is at least 20-fold. In one embodiment, the reduction in affinity is at least 30-fold. In one embodiment, the reduction in affinity is at least 40-fold. In one embodiment, the reduction in affinity is at least 50-fold. In one embodiment, the reduction in affinity is at least 100-fold. SPR may be carried out as described herein.
28. An antibody or fragment (optionally according to any preceding Clause) which competes for binding to human BMP6 with the antibody of any preceding Clause.

Optionally, competition is determined by surface plasmon resonance (SPR) or ELISA. The skilled person will be familiar with these techniques and standard conditions, for example.

In one embodiment, the antibody or fragment competes (e.g. in a dose-dependent manner) with hBMP6 (or a fusion protein thereof) for binding to cell surface-expressed hBMP6. In one embodiment, the antibody or fragment competes (e.g. in a dose-dependent manner) with hBMP6 (or a fusion protein thereof) for binding to soluble hBMP6.

Optionally, the competition for binding to hBMP6 is conducted using SPR. SPR may be carried out as described herein.
29. The antibody or fragment according to any preceding Clause which specifically binds to human BMP6 comprising SEQ ID NO: 562; and/or a cynomolgus BMP6 comprising SEQ ID NO: 564; and/or a rat BMP6 comprising SEQ ID NO: 563.

Optionally, the antibody or fragment of the invention specifically binds to the amino acid sequence of SEQ ID NO: 562. Optionally, the antibody or fragment of the invention specifically binds to the amino acid sequence of SEQ ID NO: 563. Optionally, the antibody or fragment of the invention specifically binds to the amino acid sequence of SEQ ID NO: 564.

In an example, BMP6 herein is a human, mouse or cynomolgus monkey BMP6.

In one embodiment, the antibody or fragment binds to cynomolgus BMP6 with an affinity of less than 1 nM (e.g. from 1 nM to 0.01 pM or from 1 nM to 0.1 pM, or from 1 nM to 1 pM). In one embodiment, the antibody or fragment binds to cynomolgus BMP6 with an affinity of less than 10 nM (e.g. from 10 nM to 0.01 pM or from 10 nM to 0.1 pM, or from 10 nM to 1 pM). In one embodiment, the antibody or fragment binds to cynomolgus BMP6 with an affinity of less than 0.1 nM (e.g. from 0.1 nM to 0.01 pM or from 0.1 nM to 0.1 pM, or from 0.1 nM to 1 pM). In one embodiment, the antibody or fragment binds to cynomolgus BMP6 with an affinity of less than 0.01 nM (e.g. from 0.011 nM to 0.01 pM or from 0.01 nM to 0.1 pM).

In one embodiment, the antibody or fragment binds to cynomolgus BMP6 with an affinity of within 2-fold of the affinity to hBMP6. In one embodiment, the antibody or fragment binds to cynomolgus BMP6 with an affinity of within 4-fold of the affinity to hBMP6. In one embodiment, the antibody or fragment binds to cynomolgus BMP6 with an affinity of within 5-fold of the affinity to hBMP6. In one embodiment, the antibody or fragment binds to cynomolgus BMP6 with an affinity of within 6-fold of the affinity to hBMP6. In one embodiment, the antibody or fragment binds to cynomolgus BMP6 with an affinity of within 8-fold of the affinity to hBMP6. In one embodiment, the antibody or fragment binds to cynomolgus BMP6 with an affinity of within 10-fold of the affinity to hBMP6. “hBMP6” herein is a human BMP6, eg, a human BMP6 disclosed herein, eg, comprising SEQ ID NO: 562.

In one embodiment, the antibody or fragment does not detectably bind to cynomolgus BMP6. In one embodiment, the antibody or fragment does not detectably bind to murine (eg, mouse and/or rat) BMP6.

In one embodiment, the antibody or fragment binds to murine (eg, mouse and/or rat) BMP6 with an affinity of less than 1 nM (e.g. from 1 nM to 0.01 pM or from 1 nM to 0.1 pM, or from 1 nM to 1 pM). In one embodiment, the antibody or fragment binds to murine BMP6 with an affinity of less than 10 nM (e.g. from 10 nM to 0.01 pM or from 10 nM to 0.1 pM, or from 10 nM to 1 pM). In one embodiment, the antibody or fragment binds to murine BMP6 with an affinity of less than 0.1 nM (e.g. from 0.1 nM to 0.01 pM or from 0.1 nM to 0.1 pM, or from 0.1 nM to 1 pM). In one embodiment, the antibody or fragment binds to murine BMP6 with an affinity of less than 0.01 nM (e.g. from 0.011 nM to 0.01 pM or from 0.01 nM to 0.1 pM).

Optionally, the antibody or fragment comprises an effector-enabled or effector-disabled constant region, such as a human constant region, for example an effector-null human constant region, e.g. an IgG4 constant region or an IgG1 constant region, optionally wherein the constant region is IgG4-PE, or a disabled IgG1. Optionally, the antibody or fragment comprises a murine (eg, mouse and/or rat) constant region. Optionally, the antibody or fragment comprises any of the heavy chain constant region sequences described herein.

Optionally, the constant region has CDC and/or ADCC activity.
30. The antibody or fragment according to any preceding Clause, wherein the antibody or fragment comprises a human constant region, e.g. an IgG4 constant region or an IgG1 constant region.

For example, the constant region comprises a heavy chain constant region, the heavy chain constant region comprising the amino acid sequence of SEQ ID NO: 429, 431, 433, 435, 437, 439, 440, 442, 444, 446, 448, 450, 454 or 456. Optionally, the heavy chain C region is a IGHG1 C region comprising the amino acid sequence of SEQ ID NO: 429, 431, 433, 435 or 437. Optionally, the heavy chain C region is a IGHG2 C region comprising the amino acid sequence of SEQ ID NO: 439, 440, 442 or 444. Optionally, the heavy chain C region is a IGHG4 C region comprising the amino acid sequence of SEQ ID NO: 446, 448, 450, 454 or 456, preferably SEQ ID NO: 454, preferably SEQ ID NO: 456. In an example, the heavy chain C region is encoded by a nucleic acid comprising SEQ ID NO: 451, 452 or 453.

In an example (optionally in addition to the heavy chain region as per the paragraph immediately above), the constant region comprises a light chain constant region, the light chain constant region comprising the amino acid sequence of SEQ ID NO: 458, 460, 462, 464, 466, 468, 470, 473, 476, 478, 480, 482, 484, 486, 488 or 490. Optionally, the light chain C region is a IGKC C region comprising the amino acid sequence of SEQ ID NO: 458, 460, 462, 464 or 466, preferably SEQ ID NO: 458.

Optionally, the light chain C region is a IGLC C region comprising the amino acid sequence of SEQ ID NO: 468, 470, 473, 476, 478, 480, 482, 484, 486, 488 or 490.
31. The antibody or fragment according to Clause 30, wherein the constant region is an IgG4-PE constant region.

Optionally, the antibody or fragment comprises a heavy chain constant region, wherein the constant region comprises the amino acid sequence of SEQ ID NO: 454.

The anti-BMP6 antibody or fragment according to the invention may comprise a constant region, such as a human constant region, for example an effector-null human constant region, e.g. an IgG4 constant region or an IgG1 constant region, optionally wherein the constant region is IgG4-PE, or a disabled IgG1 as defined in the sequence table herein.

In other embodiments, the antibody or fragment is any of the isotypes or constant regions as defined herein. In one embodiment, the constant region is wild-type human IgG1. For example, the constant region is an effector-enabled IgG1 constant region, optionally having ADCC and/or CDC activity. In one embodiment, the constant region is engineered for enhanced ADCC and/or CDC and/or ADCP. In another embodiment, the constant region is engineered for enhanced effector function.

The IgG4 constant region may be any of the IgG4 constant region amino acid sequences or encoded by any of the nucleic acid sequences of the sequence table herein. A heavy chain constant region may be an IgG4 comprising both the Leu235Glu mutation and the Ser228Pro mutation. This “IgG4-PE” heavy chain constant region (see the sequence table for an example) is effector null.

An alternative effector null human constant region is a disabled IgG1 being an IgG1*01 allele comprising the L235A and/or G237A mutations (e.g. LAGA, see the sequence table). In one embodiment, the antibodies or antibody fragments disclosed herein comprise an IgG1 heavy chain constant region, wherein the sequence contains alanine at position 235 and/or 237 (EU index numbering).

The potency of Fc-mediated effects may be enhanced by engineering the Fc domain by any of the techniques as will be apparent to the skilled person. In another embodiment, the antibodies and fragments disclosed herein may comprise a triple mutation (M252Y/S254T/T256E) which enhances binding to FcRn.
32. The antibody or fragment according to any preceding Clause (eg, a bispecific antibody), further comprising an antigen-binding site that specifically binds another target antigen (eg, human hemojuvelin, transferrin receptor (eg, TFR2) or a BMP receptor (eg, BMPRI or BMPRII) or binds BMP6 and another BMP (eg, BMP2, 4, 7 or 9).

For example, the bispecific antibody specifically binds to BMP6 and BMP2. For example, the bispecific antibody specifically binds to BMP6 and HJV. For example, the bispecific antibody specifically binds to BMP6 and BMPRI (ie, BMPR1, eg, BMPR1A or BMPR1B). For example, the bispecific antibody specifically binds to BMP6 and BMPRII. For example, the bispecific antibody specifically binds to BMP6 and 2. For example the bispecific antibody specifically binds to BMP6 and 4. For example the bispecific antibody specifically binds to BMP6 and 9. For example the bispecific antibody specifically binds to BMP6 and 7. For example the bispecific antibody specifically binds to BMP6 and TFR2.

In an example, the further binding site is an agonist binding site for said another antigen. In an example, the further binding site is an antagonist binding site for said another antigen.

In an example, the further binding site is an antibody binding site comprising a VH and a VL; a binding site comprised by a constant domain of the antibody (eg, an Fcab binding site) or a non-immunoglobulin binding site (eg, a fibronectin domain). Optionally, the antigen-binding site is any antigen-binding site disclosed herein.

For example, the antibody or fragment is a bispecific antibody or fragment. For example, the antibody or fragment is a dual binding antibody or fragment, or a fusion protein comprising an antibody or fragment thereof as defined in any preceding Clause. A dual binding antibody has the meaning as set out above.

In an example, the antibody, fragment or fusion protein of Clause 24 or 24a comprises a bispecific format selected from DVD-Ig, mAb², FIT-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular mAb², knob-in-holes, knob-in-holes with common light chain, knobs-in-holes with common light chain and charge pairs and FIT-Ig, e.g. mAb² and FIT-Ig.

In one embodiment, the bispecific format is selected from DVD-Ig, mAb², FIT-Ig, mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody.

In one embodiment, the bispecific format is selected from DVD-Ig, FIT-Ig, mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, FIT-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular knob-in-holes, knob-in-holes with common light chain, knobs-in-holes with common light chain and charge pairs and FIT-Ig, e.g. FIT-Ig.

In one embodiment, the bispecific format is selected from DVD-Ig, mAb², mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, mAb², mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular mAb², knob-in-holes, knobs-in-holes with common light chain and charge pairs, and knob-in-holes with common light chain, e.g. mAb².

In one embodiment, the bispecific format is selected from DVD-Ig, mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular knob-in-holes, knobs-in-holes with common light chain and charge pairs, and knob-in-holes with common light chain.
33. An anti-BMP6 antibody or fragment as defined in any preceding Clause for treating or preventing a BMP6-mediated disease or condition (eg, anaemia) in a subject.

In an example, the subject is a human. In an alternative, the subject is a non-human animal. In an example, the subject is an adult human. In an example, the subject is a paediatric human. In an example, the subject is a human CKD patient on dialysis treatment. In an example, the subject is a human having end-stage renal disease.

In an example, the antibody or fragment herein is for treating or preventing a disease or condition in a subject (eg, a human) selected from anaemia, pulmonary arterial hypertension (PAH) (eg, primary PAH or secondary PAH), Cerebral Cavernous Malformation (CCM) (eg, familial CCM or sporadic CCM), Restless Legs Syndrome (RLS), cancer (eg, breast cancer, pancreatic cancer, colorectal cancer, salivary gland cancer, oesophageal cancer or melanoma), cancer mestasis, systemic sclerosis, Sjögren's Syndrome, Endothelial to Mesenchymal Transformation (EndoMT), cardiovascular disease, atherosclerosis, Systemic Sclerosis-associated pulmonary fibrosis and cardiac fibrosis.

Examples of a disease or condition mediated by EndoMT are cardiovascular disease, atherosclerosis, Systemic Sclerosis-associated pulmonary fibrosis, cardiac fibrosis, PAH, tumour formation, tumour invasion, tumour mestasis, fibrotic disease and the generation of Carcinoma-Associated Fibroblasts (eg, in pancreatic cancer).

In an example, the disease or condition is in a human. In an example, the disease or condition is in an animal.

In an example, the antibody or fragment of the invention is for treating or preventing a TIGIT mediated disease or condition in a human, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

In an example, the BMP6-mediated disease or condition is a neurodegenerative disease, disorder or condition, e.g. selected from Alzheimer's disease, amyotrophic lateral sclerosis, Parkinson's disease, Huntington's disease, primary progressive multiple sclerosis, secondary progressive multiple sclerosis, corticobasal degeneration, Rett syndrome, a retinal degeneration disorder selected from age-related macular degeneration and retinitis pigmentosa; anterior ischemic optic neuropathy, glaucoma, uveitis, depression, trauma-associated stress or post-traumatic stress disorder, frontotemporal dementia, Lewy body dementias, mild cognitive impairments, posterior cortical atrophy, primary progressive aphasia and progressive supranuclear palsy or aged-related dementia, in particular, the neurodegenerative disease, disorder or condition is selected from Alzheimer's disease, amyotrophic lateral sclerosis, Parkinson's disease and Huntington's disease, for example, Alzheimer's disease.

In an example, the antibody, fragment, combination of the invention is administered intravenously to the subject; or is for administration intravenously to the subject. In an example, the antibody, fragment, combination of the invention is administered subcutaneously to the subject; or is for administration subcutaneously to the subject.
34. The antibody or fragment of Clause 33, wherein the antibody or fragment is administered to the subject simultaneously or sequentially with an erythropoietin stimulating agent (ESA).
35. A combination of an amount of an anti-BMP6 antibody or fragment and an amount of an ESA (eg, comprising multiple doses of said antibody and/or ESA), wherein the antibody or fragment is according to any one of Clauses 1 to 34.

There is also provided: A medical kit comprising the combination, a first sterile container comprising said amount of antibody or fragment, and a second sterile container comprising said amount of ESA, and optionally instructions for using the combination to treat anaemia in a subject.

In an example, the combination is for treating or preventing anaemia in a subject, wherein over a 4 consecutive week period a total dose of the antibody and total dose of ESA are administered to said subject in a ratio of X:Y, wherein X is from 10 to 2×10⁶ and Y=4, eg, X is from 10 to 2×10⁶ micrograms and Y=4 micrograms.

In an example the treatment increases (or is for increasing) in the subject one, more or all of Hb concentration, mean corpuscular haemoglobin (MCH) and transferrin saturation. The skilled addressee will be familiar with these parameters and how to determine them, eg, using one or more serum samples of the subject. For example, transferrin saturation, measured as a percentage, is the value of serum iron divided by the total iron-binding capacity.

In an example, said subject at the start of treatment suffers from anaemia of chronic disease (ACD) and optionally wherein the anaemia is associated with chronic inflammation (eg, the subject suffers from arthritis) or a bacterial infection (eg, Streptococcus infection), or wherein the subject is a chronic kidney disease (CKD) patient.
36. The antibody, fragment or combination according to any one of Clauses 1 to 34 for use in a method of:
   (a) preventing the blood haemoglobin level of a subject from decreasing to less than 10 g/dL, the method comprising administering the antibody or fragment and an erythropoiesis stimulating agent (ESA) to the subject;
   (b) raising blood haemoglobin to a level of at least 10 g/dL in a subject suffering from anaemia, the method comprising administering the antibody or fragment and an erythropoiesis stimulating agent (ESA) to the subject, wherein said anaemia is treated;
   (c) treating or preventing anaemia in a subject suffering from an inflammatory disease or condition, the method comprising administering the antibody or fragment and an erythropoiesis stimulating agent (ESA) to the subject, wherein said anaemia is treated or prevented;
   (d) eliminating or reducing the need to administer iron or blood transfusion to a subject suffering from anaemia, the method comprising administering the antibody or fragment and an erythropoiesis stimulating agent (ESA) to the subject, wherein said need is eliminated or reduced;
   (e) treating or preventing anaemia in a subject suffering from a microbial infection, the method comprising administering an anti-BMP6 antagonist and an erythropoiesis stimulating agent (ESA) to the subject;
   (f) reducing administration of an erythropoiesis stimulating agent (ESA) to a subject suffering from anaemia for treating anaemia, the method comprising administering the antibody or fragment and said ESA, wherein anaemia is treated in the subject; or
   (g) treating or reducing the risk of anaemia in a subject suffering from or at risk of anaemia, the method comprising administering the antibody or fragment and a low dose of an erythropoiesis stimulating agent (ESA) to the subject, wherein anaemia is treated or the risk of anaemia is reduced in the subject.
37. The antibody, fragment or combination of any one of Clauses 1 to 34 and 36, wherein the ESA is
   a. Epoetin alfa and is administered at a weekly dose of less than 1000, 1500, 2500, 5000, 11000, 18000, 34000 or 90000 units, optionally wherein the subject has previously received a weekly epoetin alfa treatment of <1500, 1500 to 2499, 2500 to 4999, 5000 to 10999, 11000 to 17999, 18000 to 33999, 34000 to 89999 or ≥90000 units respectively;
   b. darbepoetin alfa or Aranesp® and is administered at a weekly dose of less than 6.25, 10, 12.5, 20, 25, 40, 60, 100 or 200 mcg, optionally wherein the subject has previously received a weekly darbepoetin alfa or Aranesp® treatment of 6.25, 10, 12.5, 20, 25, 40, 60, 100 or 200 mcg respectively; or
   c. darbepoetin alfa or Aranesp® and is administered at a weekly dose of less than 6.25, 10, 20, 40, 60, 100 or 200 mcg, optionally wherein the subject has previously received a weekly Epoetin alfa treatment of 1500 to 2499, 2500 to 4999, 5000 to 10999, 11000 to 17999, 18000 to 33999, 34000 to 89999 or ≥90,000 units respectively.
38. The antibody, fragment or combination of any one of Clauses 1 to 34, 36 and 37 for maintaining or raising blood haemoglobin level to at least 10 g/dL in the subject at least 13 or 14 days after the subject has received the anti-BMP6 antibody or fragment and ESA.
39. Use of the antibody, fragment or combination as defined in any preceding Clause in the manufacture of a medicament for administration to a subject for treating or preventing a BMP6-mediated disease or condition, e.g. anaemia.
40. A method of treating or preventing a BMP6-mediated disease or condition in a subject (e.g. anaemia), the method comprising administering to said subject a therapeutically effective amount of an antibody, fragment or combination as defined in any one of Clauses 1 to 38, wherein the BMP6-mediated disease or condition is thereby treated or prevented.

The disease or condition can be any disclosed herein.
41. The use according to Clause 39 or the method according to Clause 40, wherein the BMP6-mediated disease or condition is anaemia.
42. The antibody, fragment, combination, use or the method according to any one of Clauses 33 to 41, further comprising administering to the subject a further therapy, for example a further therapeutic agent, optionally wherein the further therapeutic agent is selected from the group consisting of:
   (a) Intravenous iron;
   (b) An ESA (eg, an EPO);
   (c) An ActRIIa inhibitor;
   (d) An ActRIIb inhibitor;
   (e) An IL-6 or IL-6 receptor inhibitor (eg, an anti-IL-6 or IL-6 receptor antibody);
   (f) A TNF-alpha or TNF-alpha receptor inhibitor (eg, an anti-TNF-alpha or TNF-alpha receptor antibody);
   (g) A HJV inhibitor (eg, an anti-HIV antibody);
   (h) A BMP inhibitor (eg, a further anti-BMP antibody or fragment), eg, wherein the BMP is BMP2, 4, 5, 6, 7 or 9;
   (i) A matriptase-2 (MTP2) agonist (eg, a matriptase-2 (MTP2) agonist antibody);
   (j) A HIF-PH inhibitor;
   (k) A transferrin receptor 2 (TFR2) inhibitor;
   (l) A HFE inhibitor;
   (m) A NRf2 inhibitor;
   (n) A transforming growth factor-beta superfamily type I activin receptor-like kinase (ALK) receptor inhibitor;
   (o) An activin receptor inhibitor (eg, an activin receptor Fc fusion);
   (p) A GDF11 inhibitor; and
   (q) A myostatin inhibitor.

Optionally, the further agent is Luspatercept™ or Sotatercept™. Optionally, the further agent is a TGF-β Superfamily inhibitor. In an example, the further agent is a transforming growth factor-beta superfamily type I activin receptor-like kinase (ALK) receptor inhibitor; ALK 2 inhibitor, ALK3 inhibitor; ALK4 inhibitor; ALK5 inhibitor; or ALK7 inhibitor.

In an example the further agent is an IL-6 or IL-6R inhibitor, eg, Sarilumab, Vobarilizumab or tocilizumab (eg, Kevzara® or Actemra®).

In an example the further agent is a TNF-alpha or TNF-alpha receptor inhibitor, eg, adalimumab, HUMIRA®, REMICADE® or ENBREL®, SIMPONI®.

In an embodiment, the NRf2 inhibitor improves efficacy of anti-BMP6 antibody or fragment by breaking the feedback loop of more iron inducing more BMP6 expression in the subject being treated.

The disclosure includes generic versions of the branded drugs instead and the disclosure of these generic drugs is included by reference herein for possible use in the invention, eg, as part of a combination.

In an example, the combination comprises inhibitors of BMP6 and HJV; or BMP6 and HFE; or BMP6 and TFR2; or BMP6 and BMP2; or BMP6 and BMP4; or BMP6 and ALK2, wherein the BMP6 inhibitor comprises the antibody or fragment of the invention.
43. A pharmaceutical composition comprising an antibody, fragment or combination as defined in any one of Clauses 1 to 38 and 42 and a pharmaceutically acceptable excipient, diluent or carrier and optionally in combination with a further therapeutic agent selected from those mentioned above (eg, in Clause 42).
44. The pharmaceutical composition according to Clause 43 for treating and/or preventing a BMP6-mediated condition or disease, e.g. anaemia.

Suitable diseases and conditions include anaemia, pulmonary arterial hypertension (PAH) (eg, primary PAH or secondary PAH), Cerebral Cavernous Malformation (CCM) (eg, familial CCM or sporadic CCM), Restless Legs Syndrome (RLS), cancer (eg, breast cancer, pancreatic cancer, colorectal cancer, salivary gland cancer, oesophageal cancer or melanoma), cancer mestasis, systemic sclerosis, Sjögren's Syndrome, Endothelial to Mesenchymal Transformation (EndoMT), cardiovascular disease, atherosclerosis, Systemic Sclerosis-associated pulmonary fibrosis and cardiac fibrosis.

Examples of a disease or condition mediated by EndoMT are cardiovascular disease, atherosclerosis, Systemic Sclerosis-associated pulmonary fibrosis, cardiac fibrosis, PAH, tumour formation, tumour invasion, tumour mestasis, fibrotic disease and the generation of Carcinoma-Associated Fibroblasts (eg, in pancreatic cancer).
45. The pharmaceutical composition according to Clause 43 or 44 in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody or fragment.
46. A nucleic acid that encodes a VH domain and/or a VL domain of an antibody or fragment as defined in any one of Clauses 1 to 32.
47. A nucleic acid that encodes a VH domain comprising the amino acid sequence of a VH domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

For example, the identity is at least 85%. For example, the identity is at least 90%. For example, the identity is at least 95%.

Optionally, there is provided a nucleic acid that encodes a VH domain comprising the amino acid sequence of SEQ ID NO: 114, or an amino acid that is at least 70, 80, 85, 90, 95, 96, 97, 98 or 99% identical thereto. For example, the identity is at least 85%. For example, the identity is at least 90%. For example, the identity is at least 95%.
48. A nucleic acid that encodes a VL domain comprising the amino acid sequence of a VL domain of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; or an amino acid that is at least 70, 80, 85, 90, 95, 96, 97, 98 or 99% identical thereto.

Optionally, the nucleic acid also encodes a VH domain comprising the amino acid sequence of a VH domain of the selected antibody; or an amino acid that is at least 70, 80, 85, 90, 95, 96, 97, 98 or 99% identical thereto. For example, the identity is at least 85%. For example, the identity is at least 90%. For example, the identity is at least 95%.

Optionally, there is provided a nucleic acid that encodes a VL domain comprising the amino acid sequence of SEQ ID NO: 123, or an amino acid that is at least 70, 80, 85, 90, 95, 96, 97, 98 or 99% identical thereto. For example, the identity is at least 85%. For example, the identity is at least 90%. For example, the identity is at least 95%.
49. A nucleic acid (eg, in a host cell, eg, a CHO or HEK293 or Cos cell) comprising
   (a) a nucleotide sequence that is at least 70, 80, 85, 90, 95, 96, 97, 98 or 99% identical to the sequence of SEQ ID NO: 115, 520 or 521; and/or
   (b) a nucleotide sequence that is at least 70, 80, 85, 90, 95, 96, 97, 98 or 99% identical to the sequence of SEQ ID NO: 124, 522 or 523.

In an alternative, there is provided:

A nucleic acid (eg, in a host cell, eg, a CHO or HEK293 or Cos cell) comprising
(a) a nucleotide sequence that is at least 70, 80, 85, 90, 95, 96, 97, 98 or 99% identical to the sequence of SEQ ID NO: 115; and/or
(b) a nucleotide sequence that is at least 70, 80, 85, 90, 95, 96, 97, 98 or 99% identical to the sequence of SEQ ID NO: 124.

A nucleic acid (eg, in a host cell, eg, a CHO or HEK293 or Cos cell) comprising
(a) a nucleotide sequence that is at least 70, 80, 85, 90, 95, 96, 97, 98 or 99% identical to the sequence of SEQ ID NO: 520; and/or
(b) a nucleotide sequence that is at least 70, 80, 85, 90, 95, 96, 97, 98 or 99% identical to the sequence of SEQ ID NO: 522.

A nucleic acid (eg, in a host cell, eg, a CHO or HEK293 or Cos cell) comprising
(a) a nucleotide sequence that is at least 70, 80, 85, 90, 95, 96, 97, 98 or 99% identical to the sequence of SEQ ID NO: 521; and/or
(b) a nucleotide sequence that is at least 70, 80, 85, 90, 95, 96, 97, 98 or 99% identical to the sequence of SEQ ID NO: 523.

A combination of first and second nucleic acids (eg, in a host cell, eg, a CHO or HEK293 or Cos cell) respectively comprising
(a) a nucleotide sequence that is at least 70, 80, 85, 90, 95, 96, 97, 98 or 99% identical to the sequence of SEQ ID NO: 115, 520 or 521; and/or
(b) a nucleotide sequence that is at least 70, 80, 85, 90, 95, 96, 97, 98 or 99% identical to the sequence of SEQ ID NO: 124, 522 or 523.

A combination of first and second nucleic acids (eg, in a host cell, eg, a CHO or HEK293 or Cos cell) respectively comprising
(a) a nucleotide sequence that is at least 70, 80, 85, 90, 95, 96, 97, 98 or 99% identical to the sequence of SEQ ID NO: 115; and/or
(b) a nucleotide sequence that is at least 70, 80, 85, 90, 95, 96, 97, 98 or 99% identical to the sequence of SEQ ID NO: 124.

A combination of first and second nucleic acids (eg, in a host cell, eg, a CHO or HEK293 or Cos cell) respectively comprising
(a) a nucleotide sequence that is at least 70, 80, 85, 90, 95, 96, 97, 98 or 99% identical to the sequence of SEQ ID NO: 520; and/or
(b) a nucleotide sequence that is at least 70, 80, 85, 90, 95, 96, 97, 98 or 99% identical to the sequence of SEQ ID NO: 522.

A combination of first and second nucleic acids (eg, in a host cell, eg, a CHO or HEK293 or Cos cell) respectively comprising
(a) a nucleotide sequence that is at least 70, 80, 85, 90, 95, 96, 97, 98 or 99% identical to the sequence of SEQ ID NO: 521; and/or
(b) a nucleotide sequence that is at least 70, 80, 85, 90, 95, 96, 97, 98 or 99% identical to the sequence of SEQ ID NO: 523.

For example, for (a) the identity is at least 85%. For example, the identity is at least 90%. For example, the identity is at least 95%.

For example, for (b) the identity is at least 85%. For example, the identity is at least 90%. For example, the identity is at least 95%.

Herein in any instance where % identity is mentioned, in an example there is 100% identity.
50. A nucleic acid that encodes a heavy chain and/or a light chain of an antibody or fragment as defined in any one of Clauses 1 to 32.
51. A nucleic acid that encodes a heavy chain comprising an amino acid sequence that is at least 70, 80, 85, 90, 95, 96, 97, 98 or 99% identical to SEQ ID NO: 116.

For example, the identity is at least 85%. For example, the identity is at least 90%. For example, the identity is at least 95%.
52. A nucleic acid that encodes a light chain comprising an amino acid sequence that is at least 70, 80, 85, 90, 95, 96, 97, 98 or 99% identical to SEQ ID NO: 125.

For example, the identity is at least 85%. For example, the identity is at least 90%. For example, the identity is at least 95%.
53. A nucleic acid (eg, in a host cell, eg, a CHO or HEK293 or Cos cell) comprising
   (a) a nucleotide sequence that is at least 70, 80, 85, 90, 95, 96, 97, 98 or 99% identical to a heavy chain sequence selected of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713; and/or
   (b) a nucleotide sequence that is at least 70, 80, 85, 90, 95, 96, 97, 98 or 99% identical to a sequence selected of an antibody selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713.

Preferably, the selected antibody in (a) and (b) is the same antibody, eg, CL-58838. In a alternative, a first nucleic acid comprises (a) and a second nucleic acid comprises (b), eg, in a host cell, eg, a CHO or HEK293 or Cos cell.

All of the nucleic acids of the invention herein are expressible in a host cell, eg, a CHO or HEK293 or Cos cell, such as for expressing a variable domain or chain of an antibody or fragment of the invention.

For example, there is provided:

A nucleic acid (eg, in a host cell, eg, a CHO or HEK293 or Cos cell) comprising
(a) a nucleotide sequence that is at least 70, 80, 85, 90, 95, 96, 97, 98 or 99% identical to a sequence selected from SEQ ID NO: 512, 514, 516, 518 and 519; and/or
(b) a nucleotide sequence that is at least 70, 80, 85, 90, 95, 96, 97, 98 or 99% identical to a sequence selected from SEQ ID NO: 513, 515 and 517.

A nucleic acid (eg, in a host cell, eg, a CHO or HEK293 or Cos cell) comprising
(a) a nucleotide sequence that is at least 70, 80, 85, 90, 95, 96, 97, 98 or 99% identical SEQ ID NO: 512; and/or
(b) a nucleotide sequence that is at least 70, 80, 85, 90, 95, 96, 97, 98 or 99% identical to SEQ ID NO: 513.

A nucleic acid (eg, in a host cell, eg, a CHO or HEK293 or Cos cell) comprising
(a) a nucleotide sequence that is at least 70, 80, 85, 90, 95, 96, 97, 98 or 99% identical SEQ ID NO: 514; and/or
(b) a nucleotide sequence that is at least 70, 80, 85, 90, 95, 96, 97, 98 or 99% identical to SEQ ID NO: 515.

A nucleic acid (eg, in a host cell, eg, a CHO or HEK293 or Cos cell) comprising
(a) a nucleotide sequence that is at least 70, 80, 85, 90, 95, 96, 97, 98 or 99% identical SEQ ID NO: 516; and/or
(b) a nucleotide sequence that is at least 70, 80, 85, 90, 95, 96, 97, 98 or 99% identical to SEQ ID NO: 517.

A nucleic acid (eg, in a host cell, eg, a CHO or HEK293 or Cos cell) comprising
(a) a nucleotide sequence that is at least 70, 80, 85, 90, 95, 96, 97, 98 or 99% identical SEQ ID NO: 518; and/or
(b) a nucleotide sequence that is at least 70, 80, 85, 90, 95, 96, 97, 98 or 99% identical to SEQ ID NO: 513, 515 or 517.

A nucleic acid (eg, in a host cell, eg, a CHO or HEK293 or Cos cell) comprising
(a) a nucleotide sequence that is at least 70, 80, 85, 90, 95, 96, 97, 98 or 99% identical SEQ ID NO: 519; and/or
(b) a nucleotide sequence that is at least 70, 80, 85, 90, 95, 96, 97, 98 or 99% identical to SEQ ID NO: 513, 515 or 517.

For example, for (a) the identity is at least 85%. For example, the identity is at least 90%. For example, the identity is at least 95%.

For example, for (b) the identity is at least 85%. For example, the identity is at least 90%. For example, the identity is at least 95%.
54. A vector comprising the nucleic acid(s) (eg, the nucleic acid(s) of any one of Clauses 46 to 53); optionally wherein the vector is a CHO or HEK293 vector.
55. A host cell comprising the nucleic acid(s) (eg, the nucleic acid(s) of any one of Clauses 46 to 53) or the vector of Clause 54.

Optionally, the VH gene segment is selected from IGHV1-3*01 and IGHV3-11*01. Optionally, the VL gene segment is selected from IGKV1-5*03, IGKV3-20*01 and IGKV3-15*01.

In an example, the VH, DH and JH are IGHV1-3, IGHD3-10 and IGHJ4 (eg, IGHV1-3*01, IGHD3-10*01 and IGHJ4*02).

In an example, the VH, DH and JH are IGHV1-3, IGHD3-10 and IGHJ3, eg, IGHV1-3*01, IGHD3-10*01 and IGHJ3*02.

In an example, the VH, DH and JH are IGHV3-11, IGHD6-19 and IGHJ4, eg, IGHV3-11*01, IGHD6-19*01 and IGHJ4*02.

In an example, the VH, DH and JH are IGHV1-3, IGHD7-27 and IGHJ4, eg, IGHV1-3*01, IGHD7-27*02 and IGHJ4*02.

In an example, the VH, DH and JH are IGHV1-3, IGHD4-23 and IGHJ4, eg, IGHV1-3*01, IGHD4-23*01 and IGHJ4*02.

In an example, the VH, DH and JH are IGHV1-3, IGHD5-18 and IGHJ4, eg, IGHV1-3*01, IGHD5-18*01 and IGHJ4*02.

In an example, the VL and JL are IGKV1-5 and IGKJ1, eg, IGKV1-5*03 and IGKJ1*01.

In an example, the VL and JL are IGKV3-20 and IGKJ1, eg, IGKV3-20*01 and IGKJ1*01.

In an example, the VL and JL are IGKV3-15 and IGKJ3, eg, IGKV3-15*01 and IGKJ3*01.

In an example, the VL and JL are IGKV3-20 and IGKJ3, eg, IGKV3-20*01 and IGKJ3*01.

In an example, the antibody or fragment comprises a HCDR3 length of 9, 10, 11 or 12 residues, eg, 10, eg, 11. In an example, the antibody or fragment comprises a LCDR3 length of 7, 8 or 9 residues, eg, 8, eg, 9. In an example, each VH domain of the antibody or fragment comprises from 1-11 non-germline residues, eg, 1, 2, 3, 4, 5, 6, 7, 8, 9, 10 or 11 non-germline residues. In an example, each VL domain of the antibody or fragment comprises from 3-8 non-germline residues, eg, 3, 4, 5, 6, 7 or 8 non-germline residues.

In an embodiment, a CDR sequence herein is determined according to Kabat. In an alternative, the CDR sequence is determined according to IMGT.

In an example, the selected antibody is CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713 . . . . In an example, the selected antibody is CL-58838.

In an example, the selected antibody comprises the heavy chain of CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713. In an example, the selected antibody comprises the heavy chain of CL-58838.

In an example, the heavy chain of the antibody or fragment of the invention is a human gamma-1, gamma-2, gamma-3, gamma-4, mu, delta, epsilon or alpha isotype, preferably a gamma isotype (eg, an IgG4 isotype). In an example, the light chain of the antibody or fragment of the invention comprises a human kappa constant region. Alternatively, in an example, the light chain of the antibody or fragment of the invention comprises a human lambda constant region.

Optionally, the antibody is a 4-chain antibody comprising a dimer of a heavy chain associated with a dimer of a light chain. In an example, the heavy chain comprises one or heavy chain CDRs or a CDR combination as disclosed herein and/or the light chain comprises one or heavy chain CDRs or a CDR combinations as disclosed herein, such as from the same selected antibody. In an example, the heavy chain comprises a VH domain as disclosed herein and/or the light chain comprises a VL as disclosed herein, such as from the same selected antibody. In an example, the heavy chain and the light chain are from the same selected antibody, eg, any antibody disclosed in the sequence table herein or the tables in the Examples herein.

In an example, the selected antibody comprises the light chain of CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713. In an example, the selected antibody comprises the light chain of CL-58838.

In an example, the selected antibody comprises the variable domains of CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713. In an example, the selected antibody comprises the variable domains of CL-58838.

In an example, the selected antibody comprises the VH domains of CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713. In an example, the selected antibody comprises the VH domains of CL-58838.

In an example, the selected antibody comprises the VH and VL domains of CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 or CL-58713. In an example, the selected antibody comprises the VH and VL domains of CL-58838. Optionally, the VH segment is a human IGHV3-11 gene segment, eg, the VH is encoded by a nucleotide sequence that is derived from the recombination of a human IGHV3-11 and IGHJ4 (eg, human gene segments IGHV3-11*01 and IGHJ4*02; IGHV3-11, IGHD6-19, IGHJ4; or IGHV3-11*01, IGHD6-19*01 and IGHJ4*02). Optionally, the JH is a IGHJ4*02. Optionally, the VL is encoded by a nucleotide sequence that is derived from the recombination of a human VL gene segment and JL gene segment, wherein the VL gene segment is selected from IGKV1-5, IGKV3-20 and IGKV3-15. Optionally, the VL is a human IGKV3-20 (eg, IGKV3-20*01). Optionally, the JL is a IGKJ1 (eg, IGKJ1*01). For example, the VL is encoded by a nucleotide sequence that is derived from the recombination of a human IGKV3-20 (eg, IGKV3-20*01) and a human IGKJ1 (eg, IGKJ1*01).

In an example, the binding site comprises a VH/VL pair that specifically binds to human BMP6 (eg, a human BMP6 comprising or consisting of the bold sequence of SEQ ID NO: 1 in the sequence table herein). In an example, the antibody or fragment comprises 2 (eg, 2 and no more than 2) copies of the binding site.

In an example, the antibody or fragment comprises a HCDR3 length of 9-12 residues and/or the antibody or fragment comprises a LCDR3 length of 7-9 residues. In an example, the antibody or fragment comprises a HCDR3 length of 9, 10, 11 or 12 residues, eg, 10, eg, 11. In an example, the antibody or fragment comprises a LCDR3 length of 7, 8 or 9 residues, eg, 8, eg, 9. In an example, each VH domain of the antibody or fragment comprises from 1-11 non-germline residues, eg, 1, 2, 3, 4, 5, 6, 7, 8, 9, 10 or 11 non-germline residues. In an example, each VL domain of the antibody or fragment comprises from 3-8 non-germline residues, eg, 3, 4, 5, 6, 7 or 8 non-germline residues.

Optionally, the antibody or fragment competes with CL-58838 (eg, CL-58838 in IgG format, eg, IgG-PE) for binding to BMP6 (eg, human BMP6, eg, mature human BMP6, eg, BMP6 comprising or consisting of the sequence of mature BMP6 disclosed in the sequence table herein, ie, the bold sequence of SEQ ID NO: 1) as determined by SPR.

Optionally, the amino acid substitutions are conservative amino acid substitutions, optionally wherein each conservative substitution is from group (1) to (6):
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Any SPR herein is, for example, surface plasmon resonance (SPR) at 37 °C and pH 7.6.

Optionally, any BMP6 herein is (for example, in in vitro testing) human BMP6, eg, hBMP6 (Peprotech 120-06).

In an example, the antibody or fragment of the invention binds to human BMP6 with a Ka of eg, 5×10⁶ M⁻¹×s⁻¹; or about 5×10⁶ M⁻¹×s⁻¹. In an example, the antibody or fragment of the invention binds to human BMP6 with a Kd of eg, 4 or 5 s⁻¹; or about 4 or 5 s⁻¹. In an example, the antibody or fragment of the invention binds to human BMP6 with a KD of eg, 0.07 or 0.14 nM; or about 0.07 or 0.14 nM. In an embodiment, the fragment is a Fab fragment. In an embodiment, the fragment is a scFv.

In an example, the antibody comprises heavy chains, wherein each heavy chain comprises or consists of the amino acid sequence of SEQ ID NO: 116; and light chains each comprising or consisting of the amino acid sequence of SEQ ID NO: 125.

In an example, the antibody or fragment comprises heavy chain VH domains, wherein each VH comprises or consists of the amino acid sequence of SEQ ID NO: 418; and light chain VL domains each comprising or consisting of the amino acid sequence of SEQ ID NO:426.

### Alternative Antibody or Fragment:

The invention, in any of its configurations, may relate to an antibody or fragment (Alternative Antibody or Fragment) as follows.

Optionally (Option 1), the antibody or fragment comprises
a. Heavy chains each comprising or consisting of the amino acid sequence of SEQ ID NO: 403 or 566, and light chains each comprising or consisting of the amino acid sequence of SEQ ID NO: 411; or
b. Heavy chains each comprising or consisting of the amino acid sequence of SEQ ID NO: 419 and light chains each comprising or consisting of the amino acid sequence of SEQ ID NO: 427.

Optionally (Option 2), the antibody or fragment comprises
a. Heavy chains each comprising the VH domain amino acid sequence of SEQ ID NO: 402 or 565, and light chains each comprising the VL domain amino acid sequence of SEQ ID NO: 410; or
b. Heavy chains each comprising the VH domain amino acid sequence of SEQ ID NO: 418 and light chains each comprising the VH domain amino acid sequence of SEQ ID NO: 426; and
c. Optionally the heavy chains comprise a human gamma-1 (eg, IGHG1*01) or gamma-4 (eg, IGHG4*01 or IGHG4*01-PE) constant region, or the amino acid sequence of SEQ ID NO: 429, 437, 446, 454 or 456.

Optionally, the antibody or fragment competes with a reference antibody for binding BMP6, wherein the reference antibody is mAb507 (R&D Systems) or an Alternative Antibody (eg, an Option 1 or Option 2 antibody as herein defined). Competition can be by SPR or ELISA, for example, or in a functional assay such as an assay described herein (eg, in the Examples). The BMP6 can be human BMP6 (eg, mature BMP6 comprising the sequence of SEQ ID NO: 562), a rat BMP6 (eg, mature BMP6 comprising the sequence of SEQ ID NO: 56 or cynomolgus monkey BMP6 (eg, mature BMP6 comprising the sequence of SEQ ID NO: 564).

Human IgG heavy chain genes naturally encode a C-terminal lysine. This residue is mostly missing in antibodies isolated from serum and is present at low but variable levels on therapeutic antibodies expressed in mammalian cell culture systems. Since C-terminal lysine clipping occurs naturally in serum and is not known to affect overall antibody function it can be removed from the heavy chain coding sequence to provide homogeneous “lysine-clipped” heavy chains and therefore homogeneous drug product. Thus, any IgG antibody, constant region or heavy chain herein shown terminating at its C-terminus with a G can alternatively be provided in a form that ends with a GK (ie, a lysine bonded at the C-terminal side of the G shown).

### Examples of Option 1 and 2 are

Option 1a: In an example, the antibody comprises heavy chains, wherein each heavy chain comprises or consists of the amino acid sequence of SEQ ID NO: 403; and light chains each comprising or consisting of the amino acid sequence of SEQ ID NO: 411.
Option 1b: In an example, the antibody comprises heavy chains, wherein each heavy chain comprises or consists of the amino acid sequence of SEQ ID NO: 566 and light chains each comprising or consisting of the amino acid sequence of SEQ ID NO: 411.
Option 1c: In an example, the antibody comprises heavy chains, wherein each heavy chain comprises or consists of the amino acid sequence of SEQ ID NO: 419; and light chains each comprising or consisting of the amino acid sequence of SEQ ID NO: 427.
Option 2a: In an example, the antibody or fragment comprises heavy chain VH domains, wherein each VH comprises or consists of the amino acid sequence of SEQ ID NO: 402; and light chain VL domains each comprising or consisting of the amino acid sequence of SEQ ID NO: 410.
Option 2b: In an example, the antibody or fragment comprises heavy chain VH domains, wherein each VH comprises or consists of the amino acid sequence of SEQ ID NO: 565, and light chain VL domains each comprising or consisting of the amino acid sequence of SEQ ID NO: 410.

In an example, the antibody or fragment comprises heavy chain VH domains, wherein each VH comprises or consists of the amino acid sequence of SEQ ID NO: 114; and light chain VL domains each comprising or consisting of the amino acid sequence of SEQ ID NO: 123.

Optionally, the antibody or fragment competes with said reference antibody for binding to the amino acid sequence of SEQ ID NO: 1. Additionally or alternatively, optionally, the antibody or fragment competes with said reference antibody for binding to the amino acid sequence of SEQ ID NO: 492. Additionally or alternatively, optionally, the antibody or fragment competes with said reference antibody for binding to the amino acid sequence of SEQ ID NO: 491. Additionally or alternatively, optionally, the antibody or fragment competes with said reference antibody for binding the amino acid sequence of SEQ ID NO: 4. Additionally or alternatively, the antibody or fragment competes with said reference antibody for binding to the mature version of any one or more of these.

Optionally, the antibody or fragment competitively inhibits the binding of soluble haemojuvelin (HJV) to BMP6. Optionally, HJV herein is human HJV.

Optionally, the antibody or fragment does not competitively inhibit the binding of soluble haemojuvelin (HJV) to BMP6.

As used herein, “inhibits”, “inhibition”, “inhibiting” and the like, as used herein refers to the ability of an antagonist (e.g. an antibody or fragment thereof) to bind to an epitope (eg, of hBMP6) which either partially or completely prevents the binding of another antigen If the epitope to which the antagonist binds completely blocks the binding site of the ligand, then ligand binding is completely prevented (which may be a physical blocking—in the case of overlapping epitopes— or steric blocking—where the antagonist is large such that it prevents the ligand binding to its distinct epitope), and the ligand is not removed from circulation. The concentration of circulating ligand may therefore appear to be increased. If the epitope to which the antagonist binds partially blocks the binding site of the ligand, the the ligand may be able to bind, but only weakly (in the case of partial inhibition), or in a different orientation to the natural binding interaction. In this case, some of the ligand may be removed from circulation, but not as much as when the ligand binding site is completely free and available for binding. Inhibition thus refers to the physical interaction of ligand and receptor. Inhibition can be measured by HTRF, which is described in more detail elsewhere herein and in Mathis (1995) Clinical Chemistry 41(9), 1391-1397. Inhibition can also be measured by flow cytometry, where receptor is expressed on cells, or by ELISA, where receptor is adsorbed onto plates.

Optionally, the antibody comprises VH domains encoded by a VDJ region sequence, wherein the VDJ is derived from the recombination of a VH gene segment, D gene segment and JH gene segment, wherein the VH is a human germline (i) VH1-3, (ii) VH2-5 or (iii) VH3-15 gene segment. Additionally or alternatively, optionally the antibody comprises VL domains encoded by a VJ region sequence, wherein the VJ is derived from the recombination of a VL gene segment and JL gene segment, wherein the VL is a human germline (iv) Vκ3-20, (v) Vλ3-1, (vi) Vκ1-17 or (vii) Vλ1-40.

Optionally, the antibody or fragment binds to BMP6 with a stronger affinity (lower KD determined by SPR) than binding to BMP7; and/or optionally binds to BMP6 with a stronger affinity than to BMP5.

For example,
(a) the antibody or fragment binds to BMP6 with a stronger affinity (lower KD determined by SPR) than binding to BMP7; and optionally binds to BMP6 with a stronger affinity than to BMP5; and
(b) the antibody or fragment competes with a reference antibody for binding BMP6, wherein the reference antibody is mAb507 (R&D Systems) or an Alternative Antibody (eg, an Option 1 antibody or an Option 2 antibody).

Optionally, the antibody of the invention has an affinity (KD) for binding BMP6 of from 1 pM to 5 nM, optionally wherein binding is determined by SPR using a Fab of said antibody at 37 °C at pH 7.6.

Optionally, the antibody has off-rate (K_{off}) for binding BMP6 of from 1×10⁻⁵ to 1×10⁻³ S^{31 1}, optionally wherein binding is determined by SPR using a Fab of said antibody at 37 °C at pH 7.6.

Optionally, the antibody has on-rate (Kₒₙ) for binding BMP6 of from 1×10⁵ to 1×10⁷ M⁻¹S⁻¹, optionally wherein binding is determined by SPR using a Fab of said antibody at 37 °C at pH 7.6.

In an example, the antibody (eg, as a Fab) or fragment has an affinity (KD) for binding BMP6 (eg, human BMP6) of
(a) from 2, 3, 4, 5 or 10 pM to 3, 4 or 5 nM;
(b) from 1-10 pM to 5 nM;
(c) from 10 pM to 3, 4 or 5 nM;
(d) from 50 or 80 pM to 200 nM;
(e) from 50 or 80 pM to 150 nM; or
(f) from 50 or 80 pM to 100 nM.

In an example, the KD is (or is about) 5-15 pM (eg, 10 pM). In an example, the KD is (or is about) 2-5 nM (eg, 3 nM). In an example, the KD is (or is about) 100-400 pM (eg, 140 or 390 pM).

In an example, the antibody (eg, as a Fab) or fragment has an off-rate (K_{off}) for binding BMP6 (eg, human BMP6) of
(a) from 1×10⁻⁵ to 5×10⁻⁴ S⁻¹;
(b) from 1×10⁻⁵ to 6×10⁻⁴ S⁻¹;
(c) from 1×10⁻⁵ to 7×10⁻⁴ S⁻¹;
(d) from 1×10⁻⁵ to 8×10⁻⁴ S⁻¹;
(e) from 2×10⁻⁵ to 1×10⁻³ S⁻¹;
(f) from 2×10⁻⁵ to 5×10⁻⁴ S⁻¹;
(g) from 2×10⁻⁵ to 6×10⁻⁴ S⁻¹;
(h) from 2×10⁻⁵ to 7×10⁻⁴ S⁻¹; or
(i) from 2×10⁻⁵ to 8×10⁻⁴ S⁻¹.

In an example, the K_{off} is (or is about) 5×10⁻⁴ S⁻¹ (eg, when the KD is (or is about) from 2 nM to 400 pM; when the KD is (or is about) 2-5 nM (eg, 3 nM); or when the KD is (or is about) 100-400 pM (eg, 140 or 390 pM)). In an example, the K_{off} is (or is about) 3×10⁻⁵ S⁻¹ (eg, when the KD is (or is about) from 5-15 pM (eg, 10 pM)).

In an example, the antibody (eg, as a Fab) or fragment has an on-rate (Kₒₙ) for binding BMP6 (eg, human BMP6) of
(a) from 1×10⁵ to 1×10⁶ M⁻¹S⁻¹;
(b) from 1×10⁵ to 2×10⁶ M⁻¹S⁻¹;
(c) from 1×10⁵ to 3×10⁶ M⁻¹S⁻¹;
(d) from 1×10⁵ to 4×10⁶ M⁻¹S⁻¹;
(e) from 1×10⁵ to 5×10⁶ M⁻¹S⁻¹;
(f) from 2×10⁵ to 5×10⁶ M⁻¹S⁻¹;
(g) from 3×10⁵ to 5×10⁶ M⁻¹S⁻¹;
(h) from 4×10⁵ to 5×10⁶ M⁻¹S⁻¹;
(i) from 5×10⁵ to 5×10⁶ M⁻¹S⁻¹; or
(j) from 6×10⁵ to 5×10⁶ M⁻¹S⁻¹.

In an example, the Kₒₙ is (or is about) 1 or 2×10⁻⁵ M⁻¹S⁻¹ (eg, when the KD is 2-5 nM (eg, 3 nM)). In an example, the Kₒₙ is (or is about) 1-4, 1, 2, 3 or 4×10⁻⁶ M⁻¹S⁻¹ (eg, when the KD is (or is about) from 5-400 pM (eg, 140 or 390 pM) or 5-15 pM (eg, 10 pM)).

As provided in the Clauses or other aspects herein, an anti-BMP6 antibody or fragment may bind to BMP6, e.g. human BMP6 with a K_{D} of less than 50 nM, less than 40 nM, less than 30 nM as determined by surface plasmon resonance. Another embodiment, anti-BMP6 antibody or fragment may bind to BMP6, e.g. human BMP6 with a K_{D} of less than 20 nM, less than 15 nM, less than 10 nM as determined by surface plasmon resonance. The anti-BMP6 antibody or fragment may bind to BMP6, e.g. human BMP6 with a K_{D} of less than 8 nM, less than 5 nM, less than 4 nM, less than 3 nM, less than 2 nM or less than 1 nM as determined by surface plasmon resonance. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

In another embodiment, the K_{D} is within a range of 0.01 to 1 nM, or a range of 0.05 to 2 nM, or a range of 0.05 to 1 nM. The K_{D} may be with regard to hBMP6, cynomolugus monkey (ie, “cyno”) BMP6 and/or mouse BMP6.

In another embodiment, the anti-BMP6 antibodies described herein have a K_{ON} rate (e.g. as measured by SPR, e.g. at 25 °C or at 37 °C) of approximately 0.5 to 10 UM, for example approximately 1 to 8 μM or approximately 1 to 7 μM. In another embodiment, the K_{ON} rate is approximately 1 to 5 μM, e.g. approximately 1 μM, approximately 1.5 μM, approximately 2 μM, approximately 2.5 μM or approximately 3 μM. In another embodiment, the K_{ON} rate is approximately 3.5 μM, approximately 4 μM, approximately 4.5 μM, approximately 5 μM or approximately 5.5 μM.

In another embodiment, the anti-BMP6 antibodies described herein have a K_{OFF} rate (e.g. as measured by SPR, e.g. at 25 °C or at 37 °C) of approximately 0.01 to 100 mM, for example approximately 0.1 to 50 mM or approximately 0.5 to 50 mM. In another embodiment, the K_{OFF} rate is approximately 0.5 to 10 mM, or approximately 0.5 to 10 mM, e.g. approximately 1 mM, approximately 2 mM, approximately 3 mM, approximately 4 mM or approximately 5 mM. In another embodiment, the K_{OFF} rate is approximately 0.6 mM, approximately 0.7 mM, approximately 0.8 mM or approximately 0.9 mM.

The invention also provides the following method (or the antibody or fragment of the invention for use in such a method):—

A method of treating anaemia in a subject, the method comprising
(a) on an initial day (D₀) administering to the subject the anti-BMP6 antibody or fragment; and
(b) in a period of at least 3 consecutive weeks, said period starting on D₀, administering a plurality of doses of an erythropoietin stimulating agent (ESA) wherein blood haemoglobin (Hb) concentration in said subject is elevated from a baseline concentration on D₀ for the entire duration of said period,
(c) such that for the entire duration of said period:—
(i) Hb concentration is no lower than 100% of baseline Hb concentration; and during said period Hb concentration reaches at least 120% of baseline; and/or
(ii) Hb concentration is increased over baseline by at least 1 g/dl.

Optionally, the antibody, fragment or combination inhibits iron release by human liver cells, eg, in an in vitro assay or in a human. The skilled addressee will be aware of standard assays, such as those mentioned in the Examples herein.

Optionally, the antibody, fragment or combination is for treating or preventing a BMP6-mediated disease or condition as disclosed herein in a human by inhibiting iron release by human liver cells in the human. Optionally, the antibody, fragment or combination is for treating or preventing anaemia, PAH or fibrosis in a human by inhibiting iron release by human liver cells in the human.

Optionally, the antibody, fragment or combination is for treating or preventing a BMP6-mediated disease or condition as disclosed herein in a human by inhibiting hamp gene expression in human liver cells in the human. Optionally, the antibody, fragment or combination is for treating or preventing anaemia, PAH or fibrosis in a human by inhibiting hamp gene expression in human liver cells in the human.

Optionally, the antibody, fragment or combination is for treating or preventing a BMP6-mediated disease or condition as disclosed herein in a human by inhibiting hepcidin or expression thereof in human liver cells in the human. Optionally, the antibody, fragment or combination is for treating or preventing anaemia, PAH or fibrosis in a human by inhibiting hepcidin or expression thereof in human liver cells in the human.

Optionally, the antibody, fragment or combination is for treating or preventing a BMP6-mediated disease or condition as disclosed herein in a human by inhibiting BMP6 activation of hamp gene expression in the human (eg, in liver cells thereof). Optionally, the antibody, fragment or combination is for treating or preventing anaemia, PAH or fibrosis in a human by inhibiting BMP6 activation of hamp gene expression in the human (eg, in liver cells thereof).

Optionally, the antibody, fragment or combination is for treating or preventing a BMP6-mediated disease or condition as disclosed herein in a human by inhibiting HJV-mediated activation of hamp gene expression in the human (eg, in liver cells thereof). Optionally, the antibody, fragment or combination is for treating or preventing anaemia, PAH or fibrosis in a human by inhibiting HIV-mediated activation of hamp gene expression in the human (eg, in liver cells thereof). In an example, the antibody or fragment competitively inhibits HJV binding to BMP6 in vitro and/or in the human. In vitro competition may be determined by standard SPR or ELISA, for example.

In an example, the antibody or fragment inhibits human BMP6 induced luciferase expression in HepG2 cells under control of a hamp regulatory region in vitro.

Optionally, the antibody, fragment or combination is for treating or preventing a BMP6-mediated disease or condition as disclosed herein in a human by inhibiting BMP binding in the human (eg, in liver cells thereof). Optionally, the antibody, fragment or combination is for treating or preventing anaemia, PAH or fibrosis in a human by inhibiting BMP binding in the human (eg, in liver cells thereof). In an example, the antibody or fragment binds to an epitope where HIV contacts BMP6 to form a HJV-BMP6 complex which is capable of activating hamp gene expression in human liver cells.

In an example, the human cells are HepG2 cells in vitro. More details are provided in the examples herein. In an example, the inhibition is inhibition in a HepG2 cell assay in vitro, eg, as determined by inhibition of a reporter gene under control of one or more human hamp regulatory elements in vitro. For example the regulatory elements comprise response elements to pSMAD (BMP) and pSTAT (IL6). In an example, the assay is carried out using human BMP6, cynomolgus monkey BMP6, rat BMP6 or mouse BMP6; and/or the assay is carried out using human HJV, cynomolgus monkey HJV, rat HJV or mouse HJV. In an example the reporter is a luciferase gene. In an example, the antibody or fragment neutralises BMP activation of the reporter gene expression in the assay, eg, the neutralisation is at least 20, 30, 40, 50, 60, 70, 80, 90 or 95% or is complete neutralisation.

In an example, the antibody or fragment competes with a reference antibody for binding to BMP6, eg as determined by SPR, ELISA or in a HepG2 assay (eg, a HepG2 assay as described herein), eg, determined in vitro using a labelled reference antibody. In an example, competition reduces said binding by at least 20, 30, 40, 50, 60, 70, 80, 90 or 95% or is complete inhibition of the binding. For example the reference antibody is MAb507 or MAb2365; for example the reference antibody is an Alternative Antibody (eg, an Option 1 or Option 2 antibody).

In an example a or the BMP6 herein is human BMP6 (Peprotech #120-06) (SEQ ID NO: 2). In an example a or the BMP6 herein is any other human BMP6 disclosed herein.

In an example the antibody or fragment binds to human BMP6 with an off-rate of 1×10⁻⁴ S⁻¹ or lower as determined by surface plasmon resonance (SPR), eg, at room temperature or rtp. See Examples.

In an example, the antibody competes for binding to human BMP6 with a reference antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945; eg, selected from CL-58722, CL-58713, CL-58680, CL-58679, CL-58921, CL-58676, CL-58835, CL-58650 and CL-58756.

In an alternative, the reference antibody herein is selected from CL-58838, CL-66833, CL-57931, CL-57945, CL-58102, CL-58252, CL-58851, CL-75183, CL-75500, CL-75506, CL-75520, CL-75539, CL-75565, CL-75714, CL-58722, CL-58835, CL-58756, CL-58650, CL-58679, CL-58680 and CL-58713. Optionally, the reference antibody is CL-58838.

In an example, the antibody competes for binding to human BMP6 with a reference antibody, the reference antibody comprising a VH amino acid sequence selected from SEQ ID Nos: 24, 42, 114, 132, 96, 78, 60, 258, 240, 222, 204, 186, 168, 150, 276, 384, 366, 348, 294, 330 and 312 C; eg, selected from SEQ ID Nos: 24, 42, 114, 132, 96, 78 and 60; eg, selected from SEQ ID Nos: 276, 384, 366, 348, 294, 330 and 312; and/or the reference antibody comprising (eg, respectively comprising) a VL amino acid sequence selected from SEQ ID Nos: 33, 51, 123, 141, 105, 87, 69, 267, 249, 231, 213 195, 177, 159, 285, 393, 375, 357, 303, 339 and 321; eg, selected from SEQ ID Nos: 33, 51, 123, 141, 105, 87 and 69; eg, selected from SEQ ID Nos: 285, 393, 375, 357, 303, 339 and 321. In an example, the reference antibody is an IgG4 (eg, IgG4-PE) antibody. In an example, the reference antibody is an IgG1 antibody. In an example, the antibody of the invention is an IgG4 (eg, IgG4-PE) antibody. In an example, the antibody of the invention is an IgG1 antibody. In an example, the antibody of the invention binds preferentially to human BMP6 than to human BMP5 and/or human BMP7. Binding or competition may be determined, for example, by SPR or ELISA, as will be known by the skilled addressee.

In an example, the antibody competes for binding to human BMP6 with a reference antibody, the reference antibody comprising a VH amino acid sequence of a VH of an antibody selected from the sequence table herein; or selected from the tables in the Examples; or selected from CL-66833, CL-57890, 42 CL-57931, 114 CL-58838, 132 CL-58851, 96 CL-58252, 78 CL-58102, CL-57859, CL-58832, 60 CL-57945, 258 CL-75714, CL-75605, 240 CL-75565, 222 CL-75539, 204 CL-75520, CL-75519, CL-75511, 186 CL-75506, 168 CL-75500, CL-75496, CL-75194, 150 CL-75183, 276 CL-58722, 384 CL-58713, 366 CL-58680, 348 CL-58679, CL-58921, CL-58676, 294 CL-58835, 330 CL-58650 and 312 CL-58756; and/or the reference antibody comprising a VL amino acid sequence of a VH of the selected antibody. In an example, the reference antibody is an IgG4 (eg, IgG4-PE) antibody. In an example, the reference antibody is an IgG1 antibody. In an example, the antibody of the invention is an IgG4 (eg, IgG4-PE) antibody. In an example, the antibody of the invention is an IgG1 antibody. In an example, the antibody of the invention binds preferentially to human BMP6 than to human BMP5 and/or human BMP7. Binding or competition may be determined, for example, by SPR or ELISA, as will be known by the skilled addressee.

In an example, the invention antibody or fragment comprises the VH and VL domains of CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 or CL-58756.

In an example, the invention antibody or fragment comprises the VH and VL domains of CL-66833. In an example, the invention antibody or fragment comprises the VH and VL domains of CL-57931. In an example, the invention antibody or fragment comprises the VH and VL domains of CL-58838. In an example, the invention antibody or fragment comprises the VH and VL domains of CL-58851. In an example, the invention antibody or fragment comprises the VH and VL domains of CL-58252. In an example, the invention antibody or fragment comprises the VH and VL domains of CL-58102. In an example, the invention antibody or fragment comprises the VH and VL domains of CL-57945.

In an example, the selected antibody is CL-66833, CL-57931, CL-58838, CL-58851, CL-58252 or CL-58102. In an example, the selected antibody is CL-66833. In an example, the selected antibody is CL-58838.

In an example, the selected antibody comprises the variable domains of CL-66833, CL-57931, CL-58838, CL-58851, CL-58252 or CL-58102. In an example, the selected antibody comprises the variable domains of CL-66833. In an example, the selected antibody comprises the variable domains of CL-58838.

In an example, the selected antibody comprises the VH domains of CL-66833, CL-57931, CL-58838, CL-58851, CL-58252 or CL-58102. In an example, the selected antibody comprises the VH domains of CL-66833. In an example, the selected antibody comprises the VH domains of CL-58838.

In an example, the selected antibody comprises the VH and VL domains of CL-66833, CL-57931, CL-58838, CL-58851, CL-58252 or CL-58102. In an example, the selected antibody comprises the VH and VL domains of CL-66833. In an example, the selected antibody comprises the VH and VL domains of CL-58838.

In an example, the antibody or fragment of the invention comprises VH domains encoded by a nucleotide sequence that is a recombinant of human gene segments IGHV3-11 and IGHJ4 (eg, human gene segments IGHV3-11*01 and IGHJ4*02; IGHV3-11, IGHD6-19, IGHJ4; or IGHV3-11*01, IGHD6-19*01 and IGHJ4*02). Additionally or alternatively, optionally the antibody or fragment of the invention comprises VL domains encoded by a nucleotide sequence that is a recombinant of human gene segments IGKV3-20 and IGKJ1 (eg, IGKV3-20*01 and IGKJ1*01). Additionally or alternatively, optionally the antibody or fragment of the invention comprises the HCDR3 of CL-58835.

Optionally, the antibody or fragment of the invention comprises the HCDR3 of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945. Optionally, the antibody or fragment of the invention comprises the HCDR1 and/or HCDR2 of said selected antibody.

Optionally the antibody or fragment of the invention comprises the HCDR1 of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945. Optionally, the antibody or fragment of the invention comprises the HCDR2 and/or HCDR3 of said selected antibody.

Optionally the antibody or fragment of the invention comprises the HCDR2 of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945. Optionally, the antibody or fragment of the invention comprises the HCDR1 and/or HCDR3 of said selected antibody.

Optionally, the antibody or fragment of the invention comprises the VH of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945; eg, selected from CL-58722, CL-58713, CL-58680, CL-58679, CL-58921, CL-58676, CL-58835, CL-58650 and CL-58756. Optionally, the antibody or fragment of the invention comprises the VL of said selected antibody.

Optionally, the antibody or fragment of the invention comprises the VL of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945; eg, selected from CL-58722, CL-58713, CL-58680, CL-58679, CL-58921, CL-58676, CL-58835, CL-58650 and CL-58756. Optionally, the antibody or fragment of the invention comprises the VH of said selected antibody.

Optionally, the antibody or fragment of the invention comprises the heavy chain of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945; eg, selected from CL-58722, CL-58713, CL-58680, CL-58679, CL-58921, CL-58676, CL-58835, CL-58650 and CL-58756. Optionally, the antibody or fragment of the invention comprises the light chain of said selected antibody.

Optionally, the antibody or fragment of the invention comprises the light chain of an antibody selected from CL-66833, CL-57890, CL-57931, CL-58838, CL-58851, CL-58252, CL-58102, CL-57859, CL-58832, CL-57945, CL-75714, CL-75605, CL-75565, CL-75539, CL-75520, CL-75519, CL-75511, CL-75506, CL-75500, CL-75496, CL-75194, CL-75183, CL-58722, CL-58713, CL-58680, CL-58679, CL-58921, CL-58676, CL-58835, CL-58650 and CL-58756; eg, selected from CL-66833, CL-57931, CL-58838, CL-58851, CL-58252, CL-58102 and CL-57945; eg, selected from CL-58722, CL-58713, CL-58680, CL-58679, CL-58921, CL-58676, CL-58835, CL-58650 and CL-58756. Optionally, the antibody or fragment of the invention comprises the heavy chain of said selected antibody.

In an example, the selected antibody is CL-58835.

Optionally, the antibody of the invention comprises a human IgG4 constant region.

Preferably, an antibody or a fragment thereof that specifically binds to a hBMP6 does not cross-react with other antigens (but may optionally cross-react with different BMP6 species, e.g., rhesus, cynomolgus, or murine; and or may optionally cross-react with different BMPs, e.g., BMP2, 4 or 9). An antibody or a fragment thereof that specifically binds to a BMP6 antigen can be identified, for example, by immunoassays, BIAcore™, or other techniques known to those of skill in the art. An antibody or a fragment thereof binds specifically to a hBMP6 antigen when it binds to a hBMP6 antigen with higher affinity than to any cross-reactive antigen as determined using experimental techniques, such as radioimmunoassays (RIA) and enzyme-linked immunosorbent assays (ELISAs).

Typically, a specific or selective reaction will be at least twice background signal or noise and more typically more than 10 times background. See, e.g. Paul, ed., 1989, Fundamental Immunology Second Edition, Raven Press, New York at pages 332-336 for a discussion regarding antibody specificity.

Contact amino acid residues involved in the interaction of antibody and antigen, such as BMP6, may be determined by various known methods to those skilled in the art.

In one embodiment, if the antibody recognises a linear epitope, short peptides based on the antigen sequence can be produced and binding of the antibody to these peptides can be assessed using standard techniques.

In one embodiment, limited proteolytic digestion and mass spectrophotometry can be used to identify binding epitopes.

In one embodiment, the contact residues of the epitope are identified by X-ray crystallography. In one embodiment, the contact residues of the epitope are identified by cryo-electro microscopy. In one embodiment, the contact residues of the epitope are identified by a combination of limited proteolytic digestion and mass spectrometry.

In another embodiment, the anti-BMP6 antibodies (and fragments) described in herein provide improved transient expression levels over other anti-BMP6 antibodies and fragments. Thus, in one embodiment, the anti-BMP6 antibody (or fragment) is expressed in a HEK293 cell, e.g. a HEK293T cell, at an expression level of approximately 100 μg/mL, or in a range of approximately 100 to 350 μg/mL. In another embodiment, the expression level is above approximately 350 μg/mL.

In another embodiment, the anti-BMP6 antibody (or fragment) is expressed in a CHO cell, e.g. an Expi-CHO cell, at an expression level of approximately 100 μg/mL, or in a range of approximately 100 to 350 μg/mL. In another embodiment, the expression level is above approximately 350 μg/mL.

In another embodiment, the anti-BMP6 antibody (or fragment) is expressed in a CHO cell, e.g. an Expi-CHO cell or a CHO-E7 EBNA cell, at an expression level of approximately 100 μg/mL, or in a range of approximately 100 to 350 μg/mL. In another embodiment, the expression level is above approximately 350 μg/mL. The antibody for example, comprises the VH and VL domains of any one of CL-58838, formatted as a human IgG1 or human IgG4 (eg, IgG4-PE).

In any of these expression systems, the expression is carried out of a scale of between approximately 0.5 mL and 3 mL, for example between approximately 0.5 mL and 2 mL. In any of these expression systems, the anti-BMP6 antibody (or fragment) may be expressed from a pTT5 vector. In any of these expression systems, the anti-BMP6 antibody (or fragment) may be expressed in conjunction with a lipid transfection reagent, and may optionally be expressed in a CHO cell, e.g. an Expi-CHO cell. In any of these expression systems, the anti-BMP6 antibody (or fragment) may be expressed in conjunction with a PEI transfection reagent, and may optionally be expressed in a CHO cell, e.g. an CHO-E7 EBNA cell. In any of these expression systems, the anti-BMP6 antibody (or fragment) may be expressed in conjunction with a helper plasmid (e.g. an AKT helper plasmid), and may optionally be expressed in a CHO cell, e.g. an CHO-E7 EBNA cell.

In any of these expression systems, the expression level is between approximately 100 μg/mL and approximately 1500 μg/mL, for example between approximately 100 μg/mL and approximately 1000 μg/mL, or between approximately 200 μg/mL and approximately 1000 μg/mL, or between approximately 350 μg/mL and approximately 1000 μg/mL. In any of these expression systems, the lower limit of expression may be approximately 100 μg/mL, approximately 200 g/mL, approximately 300 μg/mL, or approximately 400 μg/mL. In another embodiment, the lower limit of expression may be approximately 500 μg/mL, approximately 600 μg/mL, approximately 700 μg/mL, or approximately 800 μg/mL. In any of these expression systems, the upper limit of expression may be approximately 2000 μg/mL, approximately 1800 μg/mL, approximately 1600 μg/mL, or approximately 1500 μg/mL. In another embodiment, the upper limit of expression may be approximately 1250 μg/mL, approximately 1000 μg/mL, approximately 900 μg/mL, or approximately 800 μg/mL.

In another embodiment, the expression system is a Lonza expression system, e.g. Lonza X-Ceed® system. In the Lonza expression system, the expression may be carried out at a scale of approximately 30 mL to 2 L, for example 50 mL to 1 L, or 1 L tp 2 L. In the Lonza expression system, the anti-BMP6 antibody (or fragment) may be expressed in conjunction with electroporation, and optionally without any helper plasmids. In the Lonza expression system, the anti-BMP6 antibody (or fragment) may be expressed at a level of approximately 1 g/L, or approximately 900 mg/L, or approximately 800 mg/L, or approximately 700 mg/L. In another embodiment, In the Lonza expression system, the anti-BMP6 antibody (or fragment) may be expressed at a level of approximately 600 mg/L or approximately 500 mg/L or approximately 400 mg/L. In the Lonza expression system, the anti-BMP6 antibody (or fragment) may be expressed at a level of between approximately 400 mg/L and approximately 2 g/L, for example between approximately 500 mg/L and approximately 1.5 g/L, or between approximately 500 mg/L and approximately 1 g/L. In another embodiment, the expression level is above 1 g/L. In another embodiment, the anti-BMP6 antibodies provide improved half-life over other anti-BMP6 antibodies.

In one embodiment, the antibody or fragment is a human antibody or fragment. In one embodiment, the antibody or fragment is a fully human antibody or fragment. In one embodiment, the antibody or fragment is a fully human monoclonal antibody or fragment.

in one embodiment, the antibody or fragment is a humanised antibody or fragment. In one embodiment, the antibody or fragment is a humanised monoclonal antibody or fragment.

Contact amino acid residues involved in the interaction of antibody and antigen may be determined by various known methods to those skilled in the art, such as alanine scanning, protein crystallography, mass spectrophotometry or any other technique as will be apparent to the skilled addressee.

In one embodiment, the recited CDR comprises one amino acid substitution, which may be a conservative amino acid substitution. In one embodiment, the recited CDR comprises two amino acid substitutions, which may be conservative amino acid substitutions. In one embodiment, the recited CDR comprises comprises three amino acid substitutions, which may be conservative amino acid substitutions. In one embodiment, the recited CDR comprises four amino acid substitutions, which may be conservative amino acid substitutions. In one embodiment, the recited CDR comprises five amino acid substitutions, which may be conservative amino acid substitutions. In one embodiment, the recited CDR comprises six amino acid substitutions, which may be conservative amino acid substitutions.

Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size.

Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g. substituting a charged or hydrophobic amino; acid with alanine), or alternatively, in which a naturally-occurring amino acid is substituted with a non-conventional amino acid.

In one embodiment, the conservative amino acid substitutions are as described herein. For example, the substitution may be of Y with F, T with S or K, P with A, E with D or Q, N with D or G, R with K, G with N or A, T with S or K, D with N or E, I with L or V, F with Y, S with T or A, R with K, G with N or A, K with R, A with S, K or P. In another embodiment, the conservative amino acid substitutions may be wherein Y is substituted with F, T with A or S, I with L or V, W with Y, M with L, N with D, G with A, T with A or S, D with N, I with L or V, F with Y or L, S with A or T and A with S, G, Tor V.

### Combinations

The antibody or fragment of the invention may be comprised by a combination therapy with an ESA for treating or preventing anaemia, particularly moderate to severe anaemia (ie, indicated by a blood haemoglobin of less than 9.5 g/dL). Such combination may be efficacious for treating anaemia such as ACD (Anaemia of Chronic Disease), inflammation or infection and the combination therapy may produce maintenance and elevation of blood haemoglobin concentration that is statistically significant versus use of an anti-BMP6 antibody alone. Furthermore, such effects may be durable over weeks (even after a single dose of administered anti-BMP6 antibody). Additionally, the combination therapy of the invention is useful for ESA sparing anaemia therapy, ie, enabling ESA treatment with lower than standard doses of ESA. This is useful in view of potentially harmful side-effects of ESAs. The invention also may be useful for anaemia therapy in subjects that are refractory to ESAs or have poor response to standard ESA therapy. The invention usefully can maintain blood haemoglobin outside a moderate to severe anaemia range and/or prevent decrease of blood haemoglobin to such a range. The invention, thus, is useful for reducing the need for iron or blood transfusion therapy.

The invention is useful for anaemia therapy in inflammatory disease settings and microbial infection settings.

To this end, the invention provides the following configurations 1-13:—
1. A method of maintaining a blood haemoglobin level of at least 10 g/dL in a subject, the method comprising administering an anti-BMP6 antagonist and an erythropoiesis stimulating agent (ESA) to the subject.
2. A method of preventing the blood haemoglobin level of a subject from decreasing to less than 10 g/dL, the method comprising administering an anti-BMP6 antagonist and an erythropoiesis stimulating agent (ESA) to the subject.
3. A method of raising blood haemoglobin to a level of at least 10 g/dL in a subject suffering from anaemia, the method comprising administering an anti-BMP6 antagonist and an erythropoiesis stimulating agent (ESA) to the subject, wherein said anaemia is treated.
4. A method of treating or preventing moderate or severe anaemia in a subject, the method comprising administering an anti-BMP6 antagonist and an erythropoiesis stimulating agent (ESA) to the subject, wherein said anaemia is treated or prevented.
5. A method of treating or preventing anaemia in a subject suffering from an inflammatory disease or condition, the method comprising administering an anti-BMP6 antagonist and an erythropoiesis stimulating agent (ESA) to the subject, wherein said anaemia is treated or prevented.
6. A method of eliminating or reducing the need to administer iron or blood transfusion to a subject suffering from anaemia, the method comprising administering an anti-BMP6 antagonist and an erythropoiesis stimulating agent (ESA) to the subject, wherein said need is eliminated or reduced.

In an example, one, more or all of labile plasma iron (LPI), enhanced LPI (eLPI) and non-transferrin bound iron (NTBI) are reduced in the subject. In an example, one, more or all of labile plasma iron (LPI), enhanced LPI (eLPI) and non-transferrin bound iron (NTBI) are reduced in the subject.
7. A method of treating or preventing anaemia in a subject suffering from a microbial infection, the method comprising administering an anti-BMP6 antagonist and an erythropoiesis stimulating agent (ESA) to the subject.
8. A method of reducing administration of an erythropoiesis stimulating agent (ESA) to a subject suffering from anaemia for treating anaemia, the method comprising administering an anti-BMP6 antagonist and said ESA, wherein anaemia is treated in the subject.
9. A method of treating or reducing the risk of anaemia in a subject suffering from or at risk of anaemia, the method comprising administering an anti-BMP6 antagonist and a low dose of an erythropoiesis stimulating agent (ESA) to the subject, wherein anaemia is treated or the risk of anaemia is reduced in the subject.
10. A therapeutic regimen for treating or preventing anaemia in a subject suffering from or at risk of anaemia, the regimen comprising simultaneously or sequentially administering an anti-BMP6 antagonist and an ESA to the subject, wherein
   a. On day zero the antagonist is administered to the subject; and no later than day 7 (eg, on day 1) the ESA is administered to the subject; or
   b. On day zero the ESA is administered to the subject; and no later than day 7 (eg, on day 1) the antagonist is administered to the subject; or
   c. On day zero the antagonist and the ESA are simultaneously administered to the subject; or
   d. On day zero the subject has already received the ESA and on day zero the antagonist is administered to the subject; or
   e. On day zero the subject has already received the antagonist and on day zero the ESA is administered to the subject;
   whereby at day 14 or later the blood haemoglobin level is at least 10 g/dL in the subject, wherein said anaemia is treated or prevented.
11. A combination therapy for use in a method or regimen of any preceding claim for treating or preventing anaemia in a subject, the combination comprising
a. An anti-BMP6 antagonist;
b. An ESA; and
C. Optionally instructions for use in the method or regimen.
12. An anti-BMP6 antagonist for use in in a method or regimen of any preceding configuration for treating or preventing anaemia in a subject.
13. An anti-Bone Morphogenetic Protein 6 (BMP6) antagonist for use in a method of treating or preventing anaemia in a subject, the method comprising administering said anti-BMP6 antagonist and an erythropoiesis stimulating agent (ESA) to the subject, wherein said anaemia is treated or prevented.

When an “anti-BMP6 antagonist” is mentioned herein, the antagonist may be any anti-BMP6 antibody or fragment disclosed herein, such as Alternative Antibody or fragment (as described elsewhere herein) or any antibody or fragment as claimed or in the statement of invention or mentioned in the Examples, such as in Tables 4 to 11.

In an aspect, the antagonist comprises or consists of an anti-BMP6 antibody or fragment, the method comprising
(a) on an initial day (D₀) administering to the subject the anti-BMP6 antibody or fragment; and
(b) in a period of at least 3 consecutive weeks, said period starting on D₀, administering a plurality of doses of an ESA wherein blood Hb concentration in said subject is elevated from a baseline concentration on Do for the entire duration of said period,
such that:—
(i) for the entire duration of said period Hb concentration is no lower than 100% of baseline Hb concentration; and during said period Hb concentration reaches at least 120% of baseline; and/or
(ii) for the entire duration of said period Hb concentration is increased over baseline by at least 1 g/dl.

Aspects of the invention are as follows, and these aspects (and any un-numbered paragraphs) are combinable with any other configuration, example, feature, aspect or Clause of the invention as described herein; an antagonist (eg, anti-BMP6 antibody or fragment) or ESA of the invention can be provided for use in (or can be used in) a method in the following aspects:—
1. A method of maintaining a blood haemoglobin level of at least 10, 10.5, 11, 11.5, 12, 12.5 or 13 g/dL in a subject, the method comprising administering an anti-BMP6 antagonist and an erythropoiesis stimulating agent (ESA) to the subject.
   In an example, Hb level is no more than 11, 11.5 or 12 g/dl in the subject.
2. A method of preventing the blood haemoglobin level of a subject from decreasing to less than 10, 10.5, 11, 11.5, 12, 12.5 or 13/dL, the method comprising administering an anti-BMP6 antagonist and an erythropoiesis stimulating agent (ESA) to the subject.
3. A method of raising blood haemoglobin to a level of at least 10, 10.5, 11, 11.5, 12, 12.5 or 13 g/dL in a subject suffering from anaemia, the method comprising administering an anti-BMP6 antagonist and an erythropoiesis stimulating agent (ESA) to the subject, wherein said anaemia is treated.
   In an example of any aspect, the subject is suffering from moderate or severe anaemia prior to administration of the BMP6 antagonist. An outcome of the method is, in one embodiment, that the subject does not have anaemia or has mild (and not moderate or severe) anaemia.
4. A method of treating or preventing moderate or severe anaemia in a subject, the method comprising administering an anti-BMP6 antagonist and an erythropoiesis stimulating agent (ESA) to the subject, wherein said anaemia is treated or prevented.
5. A method of treating or preventing anaemia in a subject suffering from an inflammatory disease or condition, the method comprising administering an anti-BMP6 antagonist and an erythropoiesis stimulating agent (ESA) to the subject, wherein said anaemia is treated or prevented.
   In an example, the inflammatory disease or condition is selected from the group consisting of inflammation of microbial infection (eg, a bacterial infection) or rheumatoid arthritis. In an example, the anaemia is anaemia of inflammation (also known as anaemia of chronic disease, ACD).
6. A method of eliminating or reducing the need to administer iron or blood transfusion to a subject suffering from anaemia, the method comprising administering an anti-BMP6 antagonist and an erythropoiesis stimulating agent (ESA) to the subject, wherein said need is eliminated or reduced.
   In an embodiment, the method reduces the dose (eg, weekly, fortnightly or monthly dose) or dosing frequency of iron.
7. A method of treating or preventing anaemia in a subject suffering from a microbial (eg, bacterial) infection, the method comprising administering an anti-BMP6 antagonist and an erythropoiesis stimulating agent (ESA) to the subject.
8. A method of reducing administration of an erythropoiesis stimulating agent (ESA) to a subject suffering from anaemia for treating anaemia, the method comprising administering an anti-BMP6 antagonist and said ESA, wherein anaemia is treated in the subject.
9. A method of treating or reducing the risk of anaemia in a subject suffering from or at risk of anaemia, the method comprising administering an anti-BMP6 antagonist and a low dose of an erythropoiesis stimulating agent (ESA) to the subject, wherein anaemia is treated or the risk of anaemia is reduced in the subject.
   The dose is lower than a standard dose typically used to treat or reduce anaemia in a subject, eg, a human or adult human, such as a male or female. A typical dose for treatment or prophylaxis will be readily apparent to the skilled addressee. For example, see aspect 10.
   Epogen is typically formulated in vials in multiple formulations. Single-dose vials, formulated with an isotonic sodium chloride/sodium citrate-buffered solution, are supplied in multiple strengths. Each 1 mL vial contains 2000, 3000, 4000, or 10,000 Units of epoetin alfa, Albumin (Human) (2.5 mg), citric acid (0.06 mg), sodium chloride (5.9 mg), and sodium citrate (5.8 mg) in Water for Injection, USP (pH 6.9±0.3). Single-dose 1 mL vials formulated with an isotonic sodium chloride/sodium phosphate buffer contain 40,000 Units of epoetin alfa albumin (human) (2.5 mg), citric acid (0.0068 mg), sodium chloride (5.8 mg), sodium citrate (0.7 mg), sodium phosphate dibasic anhydrate (1.8 mg), and sodium phosphate monobasic monohydrate (1.2 mg) in Water for Injection, USP (pH 6.9±0.3). Multidose, 2 mL vials contain 10,000 Units epoetin alfa, albumin (human) (2.5 mg), benzyl alcohol (1%), sodium chloride (8.2 mg), and sodium citrate (1.3 mg) per 1 ml Water for Injection, USP (pH 6.1±0.3). Multidose 1 mL vials contain 20,000 Units epoetin alfa, albumin (human) (2.5 mg), benzyl alcohol (1%), sodium chloride (8.2 mg), citric acid (0.11 mg), and sodium citrate (1.3 mg), per 1 mL in Water for Injection, USP (pH 6.1±0.3). In an example of the invention, the ESA is administered as one of these formulations.
10. The method of any preceding aspect, wherein the ESA is
   a. Epoetin alfa and is administered at a weekly dose of less than 1000, 1500, 2500, 5000, 11000, 18000, 34000 or 90000 units, optionally wherein the subject has previously received a weekly epoetin alfa treatment of <1500, 1500 to 2499, 2500 to 4999, 5000 to 10999, 11000 to 17999, 18000 to 33999, 34000 to 89999 or ≥90000 units respectively;
   b. darbepoetin alfa or Aranesp® and is administered at a weekly dose of less than 6.25, 10, 12.5, 20, 25, 40, 60, 100 or 200 mcg, optionally wherein the subject has previously received a weekly darbepoetin alfa or Aranesp® treatment of 6.25, 10, 12.5, 20, 25, 40, 60, 100 or 200 mcg respectively; or
   c. darbepoetin alfa or Aranesp® and is administered at a weekly dose of less than 6.25, 10, 20, 40, 60, 100 or 200 mcg, optionally wherein the subject has previously received a weekly Epoetin alfa treatment of 1500 to 2499, 2500 to 4999, 5000 to 10999, 11000 to 17999, 18000 to 33999, 34000 to 89999 or ≥90,000 units respectively.
11. The method of any preceding aspect, wherein the anti-BMP6 antagonist is an antibody and is administered at a total dose of no more than 30 mg/kg (eg, 0.1 to 30 mg/kg) such as every 1, 2 or 3 weeks, or every month, 2 months or 3 months. Administration can be IV or subcutaneous, eg, and the subject is a human such as a human adult.
12. A therapeutic regimen for treating or preventing anaemia in a subject suffering from or at risk of anaemia, the regimen comprising simultaneously or sequentially administering an anti-BMP6 antagonist and an ESA to the subject, wherein
   a. On day zero the antagonist is administered to the subject; and no later than day 56, 28, 14 or 7 (eg, on day 1, 6 or 7) the ESA is administered to the subject; or
   b. On day zero the ESA is administered to the subject; and no later than day 56, 28, 14 or 7 (eg, on day 1, 6 or 7) the antagonist is administered to the subject; or
   c. On day zero the antagonist and the ESA are simultaneously administered to the subject; or
   d. On day zero the subject has already received the ESA and on day zero the antagonist is administered to the subject; or
   e. On day zero the subject has already received the antagonist and on day zero the ESA is administered to the subject;
   whereby at day 14 or later (eg, at day 28, 56 or 70) the blood haemoglobin level is at least 10, 10.5, 11, 11.5, 12, 12.5 or 13 g/dL in the subject, wherein said anaemia is treated or prevented.
   Optionally the antagonist is administered for a second time no later than day 7 (eg, the antagonist is administered on day 6).
13. The method or regimen of any preceding aspect, wherein the anti-BMP6 antagonist and ESA are administered to the subject no more than 7 days apart.
14. The method or regimen of any one of any preceding aspect, wherein the method or regimen maintains blood haemoglobin level in the subject at more than 10 g/dL in the subject.
15. The method or regimen of any preceding aspect, wherein the method or regimen maintains or raises blood haemoglobin level to at least 10 g/dL in the subject at least 13 or 14 days after the subject has received the anti-BMP6 antagonist and ESA.
16. The method or regimen of aspect 14 or 15, wherein the anti-BMP6 antagonist and ESA are administered to the subject no more than 1 day apart.
17. The method or regimen of any preceding aspect, wherein the anti-BMP6 antagonist and ESA are administered to the subject simultaneously.
18. The method or regimen of any preceding aspect, wherein the blood haemoglobin level of the subject is prevented from decreasing to less than 10, 10.5, 11, 11.5, 12, 12.5 or 13 g/dL (eg, at day 14).
19. The method or regimen of any preceding aspect, wherein the blood haemoglobin of the subject is raised to a level of at least 10, 10.5, 11, 11.5, 12, 12.5 or 13 g/dL (eg, at day 14).
20. The method or regimen of any preceding aspect, wherein moderate or severe anaemia is prevented in the subject (eg, at day 14).
21. The method or regimen of any preceding aspect, wherein the subject is suffering from
   a. an inflammatory disease or condition; or
   b. an infection;
   c. kidney disease;
   d. HIV or undergoing HIV treatment; or
   e. cancer; and
   anaemia is treated or prevented in the subject.

In an example, the subject is suffering from HIV infection is HIV, hepatitis, rheumatoid arthritis, chronic kidney disease or end stage renal disease. For example, the infection is a gram-negative bacterial infection. For example, the infection is a gram-positive bacterial infection.

HIV-infected humans treated with anti-HIV therapies may develop anaemia. Thus, the invention may be useful for treating or preventing anaemia in such patients. In an example, the method or regimen treats or prevents anaemia in a HIV-infected human administered with an anti-HIV therapy, eg, administered with <4200 mg/week zidovudine.

Cancer patients treated with anti-cancer chemotherapy (eg, immunotherapy, eg, by administering an immune checkpoint inhibitor to the subject, eg, an anti-CTLA4, anti-PD-L1, anti-TIGIT, anti-ICOS or anti-PD1 antibody) may develop anaemia. Thus, the invention may be useful for treating or preventing anaemia in such patients. In an example, the method or regimen treats or prevents anaemia in a human suffering from a cancer. In the art, ESAs such as erythropoietin are typically administered to such patients at a dose of 150 units/kg IV or SC 3 times weekly initially; alternatively, 40,000 units SC once weekly until completion of chemotherapy course. In an example, the invention treats or prevents anaemia in a human cancer patient, wherein the ESA is administered to the human at less than 150 units/kg intravenously or subcutaneously 3 times weekly; or a total weekly dose of less than 450 units/kg; or less than 40,000 units subcutaneously weekly.

ESA treatment is used in the art for the reduction of need for red blood cell (RBC) transfusions in patients, eg, in patients undergoing surgery. Thus, ESA treatment is used, for example, in human patients with perioperative haemoglobin >10 g/dL but ≤13 g/dL who are at high risk for perioperative blood loss from surgery, such as elective, noncardiac, nonvascular surgery. ESA is administered at 300 units/kg SC once daily for 15 consecutive days (10 days preceding surgery, day of surgery, 4 days following surgery); alternatively, 600 units/kg SC in 4 doses administered 21, 14, and 7 days before surgery and on day of surgery. In an example, the invention treats or prevents anaemia in a human surgery patient, wherein the ESA is administered to the human at less than 300 units/kg once daily for 15 consecutive days (10 days preceding surgery, day of surgery, 4 days following surgery); or less than a total 15 day dose of 4500 units/kg; or less than 600 units/kg in 3-5 or 4 doses, eg, administered 21, 14, and 7 days before surgery and on day of surgery.
22. The method of aspect 21, wherein moderate or severe anaemia is treated or prevented in the subject.
23. The method or regimen of any preceding aspect, wherein the subject is a mammal.
24. A combination therapy for use in a method or regimen of any preceding aspect for treating or preventing anaemia in a subject, the combination comprising
   a. An anti-BMP6 antagonist;
   b. An ESA; and
   c. Optionally instructions for use in the method or regimen.
25. An anti-BMP6 antagonist for use in a method or regimen of any preceding aspect for treating or preventing anaemia in a subject.
26. The combination of aspect 24 or the antagonist of aspect 25, for treating or preventing moderate or severe anaemia.
27. The combination of antagonist of any one of aspects 24 to 26 in combination with an anti-inflammatory agent.
28. The method, regimen, combination or antagonist of any preceding aspect, wherein the antagonist comprises an anti-BMP6 antibody binding site, eg, wherein the antagonist is an antibody or anti-BMP6 trap.
   In an example, the trap comprises a human BMP6 receptor domain fused to a human antibody Fc region. In an embodiment, the Fc comprises a human gamma-1 or 4 heavy chain constant region.
29. The method, regimen, combination or antagonist of any preceding aspect, wherein the ESA is an erythropoietin.
30. The method or regimen of any one of aspects 1 to 23, 28 and 29 wherein an anti-inflammatory agent is administered to the subject.

In an example, the invention uses an anti-BMP6 monoclonal antibody (mAb) for mobilising endogenous iron stores and increasing haemoglobin synthesis and optionally also erythropoiesis. The invention, in one aspect, may reduce the need for simultaneous and prevalent use of intravenous iron or blood transfusions in ACD patients. Additionally or alternatively, the invention may reduce the dose for the underlying standard of care treatment with ESA (eg, EPO) or render ESA (eg, EPO)-non responsive patients (or those with low response) responsive to ESA co-administration with an anti-BMP6 antagonist. Additionally or alternatively, the invention may treat or prevent anaemia in patients whose anaemia is refractory or non-responsive to ESA standard of care. ESAs may be contraindicated in patients that have uncontrolled high blood pressure, or have had pure red cell aplasia (PRCA, a type of anaemia) caused by receiving an ESA (eg, darbepoetin alfa, such as Arenesp®, or eg, epoetin alfa, such as Epogen® or Procrit®.

Thus, in one embodiment of the invention the subject (eg, a human) is
i. Refractory or non-responsive to an ESA (eg, darbepoetin alfa or epoetin alfa);
ii. Suffers from or has suffered from high blood pressure (eg, uncontrolled high blood pressure); or
iii. Suffers from or has suffered from pure red cell aplasia (eg, caused by receiving an ESA, such as darbepoetin alfa or epoetin alfa).

“Refractory” in relation to drug treatment, such as ESA treatment will be readily apparent to the skilled addressee, and for example means that the subject is ESA-resistant or a low responder to the ESA (ie, has a less than average response) and is not effectively treated for anaemia by the standard of care using an ESA.

ESAs are typically used to maintain haemoglobin at the lowest level that both minimises transfusions and best meets a patient's needs. As explained above, the invention in its various configurations, aspects, examples and embodiments is useful for ESA sparing anaemia therapy, ie, enabling ESA treatment with lower than standard doses of ESA. This is useful in view of potentially harmful side-effects of ESAs. Tables A-D provide relevant information in this respect.

TABLE A
Aranesp ® dosing information

Usual Adult Dose of Aranesp for Anaemia Associated with Chronic Renal Failure:
Chronic Kidney Disease (CKD) Patients Not on Dialysis:
Initial dose: 0.45 mcg/kg IV or subcutaneously once every 4 weeks as appropriate
Comments:
Initiate treatment only when haemoglobin is less than 10 g/dL, rate of haemoglobin decline
indicates likelihood of requiring RBC transfusion, and reducing risk of alloimmunisation and/or
other RBC transfusion-related risks is a goal.
CKD Patients on Dialysis:
Initial dose: 0.45 mcg/kg IV or subcutaneously once a week or 0.75 mcg/kg once every 2 weeks
as appropriate
Comments:
Initiate treatment when haemoglobin is less than 10 g/dL.
IV route is recommended for patients on hemodialysis.
Usual Adult Dose of Aranesp for Anaemia Associated with Chemotherapy:
Initial dose: 2.25 mcg/kg subcutaneously once a week or 500 mcg subcutaneously once every 3
weeks
Duration of therapy: Until completion of chemotherapy course
Comments:
Initiate treatment if haemoglobin is less than 10 g/dL and a minimum of 2 additional months of
chemotherapy is planned.
Use the lowest dose necessary to avoid RBC transfusions.
Use: Treatment of anaemia in patients with non-myeloid malignancies where anaemia is due to
the effect of concomitant myelosuppressive chemotherapy.
Usual Pediatric Dose of Aranesp for Anaemia Associated with Chronic Renal Failure:
Less than 18 Years:
Initial dose:
Chronic Kidney Disease (CKD) Patients Not on Dialysis: 0.45 mcg/kg IV or subcutaneously
once a week or 0.75 mcg/kg once every 2 weeks
CKD Patients on Dialysis: 0.45 mcg/kg IV or subcutaneously once a week
Comments:
Initiate treatment when haemoglobin is less than 10 g/dL.
mcg = micrograms

In an example, the subject is a Chronic Kidney Disease (CKD) patient not on dialysis. In an example, the subject is a Chronic Kidney Disease (CKD) patient on dialysis. In an example, the subject is a chemotherapy patient (eg, receiving or having received chemotherapy treatment for cancer).

[Table B follows]

TABLE B
Aranesp ® Side Effects

In addition to its needed effects, some unwanted effects may be caused by darbepoetin alfa (the
active ingredient contained in Aranesp).
More common
Abdominal or stomach pain
accumulation of pus
arm, back, or jaw pain
blurred vision
breathing problems (irregular, noisy, or trouble when resting)
chest pain, discomfort, tightness, or heaviness
chills
confusion
cough producing mucus
decrease in the amount of urine
diarrhea
dilated neck veins
dizziness, fainting, or lightheadedness
dry mouth
fast, slow, or irregular heartbeat
fatigue or tiredness (extreme or unusual)
fever
headache
nausea
pain, tenderness, swelling, or warmth over injection site
pounding in the ears
rapid breathing
rapid or pounding pulse
shortness of breath
skin discoloration at the injection site
sunken eyes
sweating
swelling of the ankles, face, fingers, feet, hands, or lower legs
thirst
trouble with breathing
unconsciousness
vomiting
weight gain
wheezing
wrinkled skin
Less common
Anxiety
convulsions
difficulty with speaking (slow speech or unable to speak)
double vision
trouble with thinking
trouble with walking
unable to move the arms, legs, or face muscles (including numbness and tingling)
Rare
Fever and sore throat
hives
itching
pale skin
skin rash
unusual tiredness or weakness
Some of the side effects that can occur with darbepoetin alfa may not need medical attention. As
your body adjusts to the medicine during treatment these side effects may go away. Your health
care professional may also be able to tell you about ways to reduce or prevent some of these side
effects. If any of the following side effects continue, are bothersome or if you have any questions
about them, check with your health care professional:
More common
Constipation
general feeling of discomfort or illness
lack or loss of strength
loss of appetite
itching
pale skin
skin rash
unusual tiredness or weakness
Applies to darbepoetin alfa: injectable solution
General
The most common adverse reactions in patients with chronic kidney disease (CKD) were
hypertension, dyspnea, peripheral edema, cough, and procedural hypotension. The most common
adverse reactions in cancer patients receiving chemotherapy were abdominal pain, edema, and
thrombovascular events.
Gastrointestinal
Very common (10% or more): Nausea (up to 38%), vomiting (up to 27%), diarrhea (up to 20%),
constipation (up to 19%), abdominal pain (up to 16%)
Common (1% to 10%): Dyspepsia
Other
Very common (10% or more): Fatigue (up to 32%), fever (up to 19%), peripheral edema (up to
17%), asthenia (up to 16%), edema (up to 12.8%), procedural hypotension (10%), chest pain (up to
10%)
Common (1% to 10%): Death, influenza-like symptoms, fluid overload, fall, contusion, pain,
arteriovenous graft thrombosis
Cardiovascular
Very common (10% or more): Hypertension (up to 31%), hypotension (up to 22%)
Common (1% to 10%): Myocardial infarction, thrombotic events, angina pectoris
Frequency not reported: Arrhythmia, thromboembolism, thrombosis, thrombophlebitis
Musculoskeletal
Very common (10% or more): Myalgia (up to 20%), back pain (14%), arthralgia (up to 13%), limb
pain (up to 11%)
Common (1% to 10%): Skeletal pain
Respiratory
Very common (10% or more): Dyspnea (up to 20%), upper respiratory infection (up to 14%),
cough (up to 12%), nasopharyngitis (11%)
Common (1% to 10%): Bronchitis, sinusitis, sore throat, pulmonary embolism
Nervous system
Very common (10% or more): Headache (up to 16%), dizziness (up to 14%)
Common (1% to 10%): Cerebrovascular accident/transient ischemic attack, convulsions,
paresthesia, hypoesthesia, cerebrovascular disorders, stroke
Frequency not reported: Somnolence
Genitourinary
Very common (10% or more): Urinary tract infection (15%)
Renal
Very common (10% or more): Chronic renal failure (15%)
Metabolic
Very common (10% or more): Hypoglycemia (14%)
Psychiatric
Very common (10% or more): Insomnia (11%)
Common (1% to 10%): Depression, anxiety
Dermatologic
Common (1% to 10%): Pruritus, cellulitis, rash, skin ulcer, alopecia
Frequency not reported: Angioedema, urticaria
Local
Common (1% to 10%): Injection site pain, access hemorrhage, access infection, vascular access
thrombosis, vascular access complications
Hematologic
Common (1% to 10%): Granulocytopenia
Frequency not reported: Anaemia , pure red cell aplasia associated with neutralizing antibodies to
erythropoietin
Oncologic
Common (1% to 10%): Metastatic neoplasm
Immunologic
Frequency not reported: Serious allergic reaction, hypersensitivity reaction, anaphylactic reaction
References:
1. Cerner Multum, Inc. “Australian Product Information.”;
2. “Product Information. Aranesp (darbepoetin alfa).” Amgen, Thousand Oaks, CA;
3. Cerner Multum, Inc. “UK Summary of Product Characteristics.”

In an embodiment, the treatment or prophylaxis of the invention reduces in the subject the incidence or risk of one or more side effects listed in Table B, eg, one or more of the “common”, “more common” or “very common” side effects.

In an aspect, the invention provides a method of reduced side-effect ESA therapy of a subject suffering from or at risk of anaemia, the method comprising administering an anti-BMP6 antagonist and an erythropoiesis stimulating agent (ESA) to the subject, wherein said anaemia is treated or prevented. Optionally, the incidence or risk of one or more ESA side effects listed in Table B (eg, one or more of the “common”, “more common” or “very common” side effects) is reduced. In an example, the therapy is anaemia treatment. In an example, the therapy is anaemia prophylaxis. In an example, the anaemia is moderate or severe anaemia.

TABLE C
Aranesp ® US Boxed Warnings

CANCER: ESAs shortened overall survival and/or increased the risk of tumor progression or
recurrence in clinical studies of patients with breast, non-small cell lung, head and neck, lymphoid,
and cervical cancers. Because of these risks, prescribers and hospitals must enroll in and comply
with the ESA APPRISE Oncology Program to prescribe and/or dispense this drug to patients with
cancer. To enroll in the ESA APPRISE Oncology Program, visit world wide web.esa.apprise.com
or call 1-866-284-8089 for further assistance. To decrease these risks, as well as the risk of serious
cardiovascular and thromboembolic reactions, use the lowest dose needed to avoid RBC
transfusions. Use ESAs only for anaemia from myelosuppressive chemotherapy. ESAs are not
indicated for patients receiving myelosuppressive chemotherapy when the anticipated outcome is
cure. Discontinue following the completion of a chemotherapy course.

In an embodiment, the treatment or prophylaxis of the invention reduces in the subject the incidence or risk of one or more side effects listed in Table C, eg, shortened overall survival and/or increased risk of tumour progression or recurrence wherein the subject is a breast, non-small cell lung, head and neck, lymphoid, and cervical cancer patient; or a cardiovascular or thromboembolic reaction, such as stroke.

In an aspect, the invention provides a method of reduced side-effect ESA therapy of a subject suffering from or at risk of anaemia, the method comprising administering an anti-BMP6 antagonist and an erythropoiesis stimulating agent (ESA) to the subject, wherein said anaemia is treated or prevented. Optionally, the incidence or risk of one or more ESA side effects listed in Table C (eg, shortened overall survival and/or increased risk of tumour progression or recurrence wherein the subject is a breast, non-small cell lung, head and neck, lymphoid, and cervical cancer patient; or a cardiovascular or thromboembolic reaction, such as stroke) is reduced. In an example, the therapy is anaemia treatment. In an example, the therapy is anaemia prophylaxis. In an example, the anaemia is moderate anaemia. In an example, the anaemia is moderate to severe anaemia. In an example, the anaemia is severe anaemia. In an example, the anaemia in the invention is anaemia from myelosuppressive chemotherapy.

TABLE D
Estimated Aranesp Starting Doses (mcg/week) for Patients with CKD
on Dialysis Based on Previous Epoetin alfa Dose (Units/week)

| Previous Weekly Epoetin | Aranesp Dose (mcg/week) | |
|---|---|---|
| alfa Dose (Units/week) | Adult | Paediatric |
| <1,500 | 6.25 | * |
| 1,500 to 2,499 | 6.25 | 6.25 |
| 2,500 to 4,999 | 12.5 | 10 |
| 5,000 to 10,999 | 25 | 20 |
| 11,000 to 17,999 | 40 | 40 |
| 18,000 to 33,999 | 60 | 60 |
| 34,000 to 89,999 | 100 | 100 |
| ≥90,000 | 200 | 200 |

*For paediatric patients receiving a weekly epoetin alfa dose of <1,500 Units/week, the available data are insufficient to determine an Aranesp conversion dose.

Aspects of the invention provide (i) and (ii)
(i) A method of reducing administration of an erythropoiesis stimulating agent (ESA) to a subject suffering from anaemia for treating anaemia, the method comprising administering an anti-BMP6 antagonist and said ESA, wherein anaemia is treated in the subject.
(ii) A method of treating or reducing the risk of anaemia in a subject suffering from or at risk of anaemia, the method comprising administering an anti-BMP6 antagonist and a low dose of an erythropoiesis stimulating agent (ESA) to the subject, wherein anaemia is treated or the risk of anaemia is reduced in the subject.

In examples of these aspects the ESA is
a. Epoetin alfa and is administered at a weekly dose of less than 1000, 1500, 2500, 5000, 11000, 18000, 34000 or 90000 units, optionally wherein the subject has previously received a weekly epoetin alfa treatment of <1500, 1500 to 2499, 2500 to 4999, 5000 to 10999, 11000 to 17999, 18000 to 33999, 34000 to 89999 or ≥90000 units respectively;
b. darbepoetin alfa or Aranesp® and is administered at a weekly dose of less than 6.25, 10, 12.5, 20, 25, 40, 60, 100 or 200 mcg, optionally wherein the subject has previously received a weekly darbepoetin alfa or Aranesp® treatment of 6.25, 10, 12.5, 20, 25, 40, 60, 100 or 200 mcg respectively; or
c. darbepoetin alfa or Aranesp® and is administered at a weekly dose of less than 6.25, 10, 20, 40, 60, 100 or 200 mcg, optionally wherein the subject has previously received a weekly Epoetin alfa treatment of 1500 to 2499, 2500 to 4999, 5000 to 10999, 11000 to 17999, 18000 to 33999, 34000 to 89999 or ≥90,000 units respectively.

In examples the ESA is
(i) Epoetin alfa and is administered at a weekly dose in the range from 3000 to 30000 units; or
(ii) darbepoetin alfa or Aranesp® and is administered at a weekly dose in the range from 15 to 100 mcg; and
   wherein the subject is a human, eg, an adult human.

In an example, the blood haemoglobin is raised to or maintained at a level of more than 10 g/dL.

In an example, the subject is an adult human. In an example, the subject is a paediatric human. In an example, the subject is a human CKD patient on dialysis treatment. In an example, the subject is a human having end-stage renal disease.

A therapeutically or prophylactically effective amount of the antagonist and ESA are administered to the subject in the methods of the invention. In an example, the anti-BMP6 antagonist and ESA are administered to the subject no more than 10, 14, 21 or 28 days apart. For example, the anti-BMP6 antagonist and ESA are administered to the subject no more than 1 or 2 months apart.

Examples of Erythropoiesis-Stimulating Agents (ESAs) are epoetin alfa, Epogen®, Dynepo®, Eprex®, erythropoietin, Darbepoetin alfa, Aranesp®, Epoetin beta, NeoRecormon®, methoxy polyethylene glycol-epoetin beta, Mircera® and Procrit®. In an embodiment, the ESA of the invention is any one of these or a combination of two or more of these.

In an example, the ESA comprises or consists of recombinant erythropoietin, eg, selected from the following table. Erythropoietin has a variety of glycosylation patterns giving rise to alpha, beta, delta, and omega forms:

TABLE E
Example Erythropoietins

| | |
|---|---|
| epoetin alfa: | epoetin zeta (biosimilar |
| Darbepoetin (Aranesp ™) | forms for epoetin alpha): |
| Epocept ™ (Lupin pharma) | Silapo ™ (from Stada) |
| Nanokine ™ (Nanogen | Retacrit ™ (from Hospira) |
| Pharmaceutical biotechnology, | Miscellaneous: |
| Vietnam) | Epocept ™, made by Lupin |
| Epofit ™ (Intas pharma) | Pharmaceuticals |
| Epogen ™, made by Amgen | EPOTrust ™, made by |
| Epogin ™ | Panacea Biotec Ltd |
| Eprex ™, made by Janssen-Cilag | Erypro Safe ™, made by |
| Binocrit ™, made by Sandoz | Biocon Ltd. |
| Procrit ™ | Repoitin ™, made by Serum |
| epoetin beta: | Institute of india Limited |
| epoetin beta: | Vintor ™, made by Emcure |
| NeoRecormon ™, made | Pharmaceuticals |
| by Hoffmann-La Roche | Epofit ™, made by Intas |
| Recormon ™ | pharma |
| Methoxy polyethylene glycol- | Erykine ™, made by Intas |
| epoetin beta (Mircera ™) by Roche | Biopharmaceutica |
| epoetin delta: | Wepox ™, made by |
| Dynepo ™ trademark name for an | Wockhardt Biotech |
| erythropoiesis stimulating protein, | Espogen ™, made by LG life |
| byShire plc | sciences. |
| epoetin omega: | ReliPoietin ™, made by |
| Epomax ™ | Reliance Life Sciences |
| | Shanpoietin ™, made by |
| | Shantha Biotechnics Ltd |
| | Zyrop ™, made by Cadila |
| | Healthcare Ltd. |
| | EPIAO ™ (rHuEPO), made by |
| | Shenyang Sunshine |
| | Pharmaceutical Co.. LTD. |
| | China |
| | Cinnapoietin ™, made by |
| | CinnaGen biopharmaceutical |
| | Iran. |

In an example, the ESA of the invention is selected from the group consisting of an alpha, beta, delta, zeta and omega form.

In an example, the ESA is a hypoxia-inducible factor prolyl-hydroxylase (HIF-PH) inhibitor, eg, roxadustat or FG-4592. HIF is the primary regulator of the production of red blood cells (RBCs) in the body and a potentially novel mechanism of treating anaemia. This novel mechanism of action is referred to as hypoxia inducible factor-prolyl hydroxylase (HIF-PH) inhibitors. HIF-PH inhibitors act by simulating the body's natural response to anaemia. This allows a controlled, adaptive stimulation of the erythropoietic system in the body. This activation of the whole system results in both increased red blood cell (RBC) production and improved stabilization of the bone marrow's iron supply, which ensures the proper incorporation of iron into haemoglobin necessary for such RBC production. This adaptive simulation is very similar to the natural response that is induced when a person ascends in altitude. At higher altitudes, low levels of oxygen circulating in the bloodstream lead to reduced HIF-PH activity in relevant cells in the kidney and liver. The reduced HIF-PH activity stabilizes and increases intracellular levels of proteins HIF1α and HIF2α (referred to as HIFα collectively). For most cells the stabilization of HIF2α is greater than that of HIF1α, which ultimately leads to an increase in erythropoietin (EPO) secretion and a subsequent increase in RBC production. HIF-PH inhibitors work by blocking the effect of the prolyl hydroxylase enzymes, which promote the breakdown of HIFα proteins. As the breakdown is inhibited, the level of these HIFα proteins increases in cells. These HIFs are the primary protein mediators that enable the body and all of its individual cells to adapt to changes in levels of oxygen. Both HIFα proteins are consistently produced and their levels in cells are adjusted by the activity of the HIF-PH enzymes, which target the HIFα proteins for degradation. HIF1α helps cells survive under very low oxygen conditions, whereas HIF2α helps cells and the body to adapt to modest changes in oxygen, such that would occur with a change in altitude from sea level to up to 7500 feet. When HIFα is stabilized, it travels to the nucleus of the cell, where it binds to the protein HIFB. When bound together, they induce the genetic signal for the production of EPO and several other proteins. The HIF-PH inhibitors increase HIFα levels much in the same way that a reduction in oxygen increases HIFα levels by inhibiting the HIF-PH enzymes in the body. With continued stabilisation of HIFα (either by staying at higher altitude or by daily dosing of the HIF-PH inhibitor), the level of haemoglobin and RBCs will rise in order to increase the amount of oxygen circulating in the blood.

An example of an anti-BMP6 antibody is MAB507, that is commercially available from R&D Systems (Monoclonal Mouse IgG2B, Clone #74219). Other suitable antibodies are disclosed in U.S. Pat. No. 8,980,582, WO2016098079 and US20160176956A1 the disclosure of which (and explicitly the sequences of antibodies, variable regions and CDRs therein) are incorporated herein by reference for possible use in the present invention.

In an embodiment, the antagonist comprises or consists of an antibody, or antigen-binding fragment thereof, that binds to human BMP-6 (SEQ ID NO: 1), comprising a light chain variable region (LCVR) and a heavy chain variable region (HCVR), wherein the LCVR comprises the complementarity determining regions (CDRs) LCDR1, LCDR2, and LCDR3, and the HCVR comprises the CDRs HCDR1, HCDR2, and HCDR3, wherein the LCDR1 is the polypeptide of SEQ ID NO: 2, the LCDR2 is the polypeptide of SEQ ID NO: 3, the LCDR3 is the polypeptide of SEQ ID NO: 4, the HCDR1 is the polypeptide of SEQ ID NO: 5, the HCDR2 is the polypeptide of SEQ ID NO: 6 or SEQ ID NO: 7, and the HCDR3 is the polypeptide of SEQ ID NO: 8. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody, or antigen-binding fragment thereof, that binds to human BMP-6 (SEQ ID NO: 1), comprising a light chain variable region (LCVR) and a heavy chain variable region (HCVR), wherein the LCVR comprises the complementarity determining regions (CDRs) LCDR1, LCDR2, and LCDR3, and the HCVR comprises the CDRs HCDR1, HCDR2, and HCDR3, wherein the LCDR1 is the polypeptide of SEQ ID NO: 2, the LCDR2 is the polypeptide of SEQ ID NO: 3, the LCDR3 is the polypeptide of SEQ ID NO: 4, the HCDR1 is the polypeptide of SEQ ID NO: 5, the HCDR2 is the polypeptide of SEQ ID NO: 6, and the HCDR3 is the polypeptide of SEQ ID NO: 8. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody, or antigen-binding fragment thereof, that binds to human BMP-6 (SEQ ID NO: 1), comprising a light chain variable region (LCVR) and a heavy chain variable region (HCVR), wherein the LCVR comprises the complementarity determining regions (CDRs) LCDR1, LCDR2, and LCDR3, and the HCVR comprises the CDRs HCDR1, HCDR2, and HCDR3, wherein the LCDR1 is the polypeptide of SEQ ID NO: 2, the LCDR2 is the polypeptide of SEQ ID NO: 3, the LCDR3 is the polypeptide of SEQ ID NO: 4, the HCDR1 is the polypeptide of SEQ ID NO: 5, the HCDR2 is the polypeptide of SEQ ID NO: 7, and the HCDR3 is the polypeptide of SEQ ID NO: 8. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody, or antigen-binding fragment thereof, that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 10 or SEQ ID NO: 11. In a further embodiment, the antagonist comprises or consists of an antibody, or antigen-binding fragment thereof, that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 10. In another embodiment, the antagonist comprises or consists of an antibody, or antigen-binding fragment thereof, that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 11. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 10 or SEQ ID NO: 11. In a further embodiment, the present invention provides an antibody that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 10. In another embodiment, the antagonist comprises or consists of an antibody that binds to human BMP-6 (SEQ ID NO: 1), comprising an LCVR and an HCVR, wherein the LCVR is the polypeptide of SEQ ID NO: 9, and the HCVR is the polypeptide of SEQ ID NO: 11. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody that binds to human BMP-6 (SEQ ID NO: 1), comprising a light chain (LC) and a heavy chain (HC), wherein the LC is the polypeptide of SEQ ID NO: 12, and the HC is the polypeptide of SEQ ID NO: 13 or SEQ ID NO: 14. In a further embodiment, the antagonist comprises or consists of an antibody that binds to human BMP-6 (SEQ ID NO: 1), comprising a LC and a HC, wherein the LC is the polypeptide of SEQ ID NO: 12, and the HC is the polypeptide of SEQ ID NO: 13. In another embodiment, the antagonist comprises or consists of an antibody that binds to human BMP-6 (SEQ ID NO: 1), comprising a LC and a HC, wherein the LC is the polypeptide of SEQ ID NO: 12, and the HC is the polypeptide of SEQ ID NO: 14. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the antagonist comprises or consists of an antibody that binds to human BMP-6 (SEQ ID NO: 1), comprising two light chains and two heavy chains, wherein each light chain is the polypeptide of SEQ ID NO: 12, and each heavy chain is the polypeptide of SEQ ID NO: 13. In an embodiment, the antagonist comprises or consists of an antibody that binds to human BMP-6 (SEQ ID NO: 1), comprising two light chains and two heavy chains, wherein each light chain is the polypeptide of SEQ ID NO: 12, and each heavy chain is the polypeptide of SEQ ID NO: 14. The SEQ ID NOs in this paragraph are those disclosed in U.S. Pat. No. 8,980,582, and these sequences are explicitly incorporated herein by reference for possible use in the present invention and for possible inclusion in one or more claims herein.

In an embodiment, the present invention provides a pharmaceutical composition comprising an anti-BMP6 antagonist (eg, an antibody, or BMP6-binding fragment thereof) of the present invention, and an acceptable carrier, diluent, or excipient. More particularly, the compositions of the present invention further comprise one or more additional therapeutic agents, eg, an ESA and/or an anti-inflammatory agent. Suitable anti-inflammatory agents can be antibodies or antibody fragments, eg, an anti-TNF alpha antibody (eg, adalimumab, Humira®, infliximab, Remicade®, golimumab, Simponi®, or trap (eg, etanercept or Enbrel®); or anti-TNFR antibody or antibody fragment, or an anti-IL6R antibody (eg, sarilumab, tocilizumab or Actemra®).

In an example, the anti-BMP6 antagonist, antibody or fragment binds to BMP6 with a KD of less than about 1×10⁻⁸ M, preferably, less than about 1×10⁻⁹ M as determined by common methods known in the art, eg, by use of a surface plasmon resonance (SPR) biosensor at 37 °C.

“Effective amount” means the amount of an antagonist (eg, antibody) or ESA of the present invention or pharmaceutical composition of the present invention that will elicit the biological or medical response or desired therapeutic effect on a subject, mammal or human that is being sought by the researcher, medical doctor, or other clinician. An effective amount may vary according to factors such as the disease state, age, sex, and weight of the individual, and the ability of the antibody and/or ESA to elicit a desired response in the individual. An effective amount is also one in which any toxic or detrimental effect is outweighed by the therapeutically beneficial effects.

The terms “treatment,” “treat,” “treating,” and the like, are meant to include slowing or reversing the progression of a disorder, such as anaemia, moderate anaemia, severe anaemia or blood haemoglobin decrease. These terms also include alleviating, ameliorating, attenuating, eliminating, or reducing one or more symptoms of a disorder or condition (such as anaemia, moderate anaemia, severe anaemia or blood haemoglobin decrease), even if the disorder or condition is not actually completely eliminated. A subject or patient refers to a mammal, preferably a human with a disease, disorder or condition (eg, anaemia or at risk of anaemia) that would benefit from inhibition of BMP-6 activity. The term “preventing” is for example reducing the risk of a disease or condition, such as anaemia.

An ESA, anti-BMP6 antagonist antibody, or antigen-binding fragment thereof, of the present invention, combination or pharmaceutical composition comprising the same, may be administered by parenteral routes (eg, subcutaneous, intravenous, intraperitoneal, intramuscular, or transdermal). Administration may be to a subject alone or in combination with a pharmaceutically acceptable carrier and/or diluent in single or multiple doses. Pharmaceutical compositions, combinations or antagonists of the present invention can be prepared by methods well known in the art (e.g., Remington: The Science and Practice of Pharmacy, 19^{th} ed. (1995), A. Gennaro et al., Mack Publishing Co.) and may comprise or be combined with one or more pharmaceutically acceptable carriers, diluents, or excipients.

In an example, the subject is suffering from moderate or severe anaemia prior to administration of the BMP6 antagonist and the moderate or severe anaemia is treated. In an embodiment, the subject is suffering from moderate anaemia prior to administration and after the treatment the subject has mild or no anaemia. In an embodiment, the subject is suffering from severe anaemia prior to administration and after the treatment the subject has mild, moderate or no anaemia. In an embodiment, after the treatment the subject has mild or no anaemia, and not moderate or severe anaemia. In another embodiment, after the treatment the subject does not have anaemia. In an embodiment, the subject has a blood haemoglobin level of less than 9.5 g/dL prior to administration and after the treatment the subject has a blood haemoglobin level of at least 10, 11, 12, 13 or 14 g/dL.

Anaemia is generally considered when haemoglobin concentrations fall below 11 g/dL for pregnant women, 12 g/dL for non-pregnant women, and 13 g/dL for men.

The severity of anaemia is categorized by the following haemoglobin concentration ranges:
Mild anaemia is considered when haemoglobin is between 9.5-13.0 g/dL
Moderate anaemia is considered when haemoglobin is between 8.0-9.5 g/dL
Severe anaemia is considered for haemoglobin concentrations below 8.0 g/dL

In an example, the level of haemoglobin is at or equivalent to a measurement at sea level.

In an embodiment, the subject is a human male, eg, an adult or infant. In an embodiment, the subject is a human female, eg, an adult or infant, eg, a non-pregnant female or pregnant female. I an example, the human is a dialysis patient. The infant may be a human that is >1 month old.

In an example, the method is a method of eliminating or reducing the need to administer iron or blood transfusion to a subject suffering from anaemia, Eg, for reducing the dose or dosing frequency of iron to the subject.

The invention may comprise simultaneously or sequentially administering the anti-BMP6 antagonist and ESA. In an example, antagonist and ESA are administered no more than 1 month, 4 weeks, 3 weeks, 2 weeks, 1 week, 10 days, 9 days, 8 days, 7 days, 6 days, 5 days, 4 days, 3 days, 2 days or 1 day apart. As exemplified herein, administration of the antagonist and ESA can be effective if no more than 7 days (eg, no more than one day) apart. In an example, the anti-BMP6 antagonist and ESA are administered to the subject no more than 10, 14, 21 or 28 days apart.

In an example, the ESA is administered 2, 3 or 4 times weekly. In an example, the ESA is administered 1, 2, 3 or 4 times monthly or in a 8 week period. In an example, the ESA (eg, epoetin alfa) is administered at a total dose of <3000, 2900, 2800, 2700, 2600, <2500, 2500, 2400, 2300, 2200, 2100, <2000, 2000, 1900, 1800, 1700, 1600, 1500, 1400, 1300, 1200, 1100 or 1000 units/kg per week. In another example, the ESA is administered 1, 2, 3 or 4 times monthly or in a 8 week period. In an example, the ESA (eg, darbepoetin alfa) is administered at a total dose of <15, <30, 12, 11, 10, 9, 8, 7, 6 or 5 mcg/kg per week.

In an example, the ESA and/or antagonist is administered to the subject intravenously or subcutaneously.

In an example, the anaemia is in a subject receiving or having received zidovudine treatment.

Optionally, any configuration of the invention is also for one or more of:—
(a) increasing or maintaining increased blood iron, eg, for treating or reducing the risk of anaemia;
(b) treating iron deficiency;
(c) treating or reducing the risk of Anaemia of Chronic Inflammation (ACI);
(d) treating or reducing the risk of Anaemia of Chronic Disease (ACD);
(e) treating or reducing the risk of anaemia associated with cancer, a kidney condition or GvHD;
(f) increasing blood or serum iron level;
(g) increasing reticulocyte count;
(h) increasing red blood cell count;
(i) increasing haemoglobin; and
(j) increasing haematocrit in the subject (eg, in a human).

In an embodiment, the invention is for regulating (eg, increasing) erythropoiesis in the subject.

In an embodiment, the subject is a human comprising BMP6 gene SNP rs111588693. This may be correlated with increased propensity for anaemia.

In an example, the anaemia is anaemia of chronic disease (ACD), such as anaemia of cancer, or anaemia of chronic kidney disease (CKD). Certain chronic diseases, such as cancer, kidney disease, and autoimmune disorders, can lead to ACD when overactive inflammatory cytokines cause dysregulation of iron homeostasis, reduction of erythropoiesis, and a decrease in the life span of red blood cells. Hepcidin has been identified as a key hormone involved in iron homeostasis; high levels of hepcidin have been associated with the iron restricted erythropoiesis seen in ACD. BMP-6 has been shown to increase hepcidin expression. In an example, the invention is for reducing or maintaining reduced hepcidin level in the subject.

Anaemia of CKD is anaemia that is an early and common complication in patients suffering with CKD. Anaemia of cancer is anaemia caused by haematological malignancies and some solid tumours; whereas, chemotherapy-induced (eg, immunotherapy-induced) anaemia is anaemia caused by the treatment of cancer patients with chemotherapeutic agents. Anaemia in CKD exacerbates diabetic neuropathy, cardiovascular disease, and retinopathy, among other conditions. Cancer-related anaemia is associated with an increased relative risk of death. Current treatment options for cancer-related anaemia are limited to blood transfusions, as erythropoiesis-stimulating agents are only indicated for chemotherapy-induced anaemia.

In an example, the subject is suffering from a chronic disease, such as cancer (eg, a haematological malignancy or a solid tumour), kidney disease, an autoimmune disorder or chemotherapy-induced anaemia. In an example, the subject (eg, human) is suffering from CKD and one or more of diabetic neuropathy, cardiovascular disease and retinopathy.

In an example, the anaemia is hepcidin related iron restricted anaemia. In an example, the anaemia is iron refractory iron deficiency anaemia (IRIDA). In an embodiment, the IRIDA is caused by a defect in the TMPRSS6 gene of the subject, eg, wherein IRIDA is caused by a TMPRSS6 gene mutation (eg, a SNP, such as rs855791; rs2543519; rs2235324; or rs1421312).

In an example, the method is a method of treating or preventing Sjögren's syndrome in addition to or instead of treating or preventing anaemia.

In an example, the invention is for increasing blood iron level, serum iron level, reticulocyte count, red blood cell count, haemoglobin, and/or haematocrit in the subject (eg, in a human).

In an embodiment, the present invention provides the use of an anti-BMP6 antagonist and an ESA for the manufacture of a medicament. In a further embodiment, the present invention provides the use of an anti-BMP6 antagonist and an ESA for the manufacture of a medicament for the treatment or prevention of anaemia, eg, moderate to severe anaemia. In another embodiment, the present invention provides the use of an anti-BMP6 antagonist and an ESA for the manufacture of a medicament for the treatment of anaemia of chronic disease. In another embodiment, the present invention provides the use of an anti-BMP6 antagonist and an ESA for the manufacture of a medicament for the treatment of anaemia of chronic kidney disease. In another embodiment, the present invention provides the use of an anti-BMP6 antagonist and an ESA for the manufacture of a medicament for the treatment of anaemia of cancer. In an embodiment, the present invention provides the use of an anti-BMP6 antagonist and an ESA for the manufacture of a medicament for the treatment of IRIDA. In a further embodiment, the present invention provides the use of an anti-BMP6 antagonist and an ESA for the manufacture of a medicament for the treatment of IRIDA, wherein IRIDA is caused by a TMPRSS6 gene mutation (eg, a SNP, such as rs855791; rs2543519; rs2235324; or rs1421312). In an embodiment, the present invention provides the use of an anti-BMP6 antagonist and an ESA for the manufacture of a medicament for the treatment of Sjögren's syndrome.

### EMBODIMENTS

Embodiments of the invention are as follows, and these Embodiments (and any un-numbered paragraphs) are combinable with any other configuration, Clause, paragraph, example, feature or aspect of the invention as described herein; an antagonist (eg, anti-BMP6 antibody or fragment) or ESA of the invention can be provided for use in (or can be used in) an method in the following Embodiments:—
1. An anti-Bone Morphogenetic Protein 6 (BMP6) antagonist for use in a method of treating or preventing anaemia in a subject, the method comprising administering said anti-BMP6 antagonist and an erythropoiesis stimulating agent (ESA) to the subject, wherein said anaemia is treated or prevented.
   In an embodiment, the method is for treating anaemia in the subject, wherein said anaemia is treated.
   In an example, the antagonist comprises or consists of an anti-BMP6 antibody or fragment, eg, a human, humanised or chimaeric antibody. In an alternative to an antibody or fragment, a different BMP6 antagonist is contemplated by the invention, eg, an anti-BMP6 trap or a HJV-Fc.
2. An antagonist according to Embodiment 1, wherein the antagonist comprises or consists of an anti-BMP6 antibody or fragment, the method comprising
   (a) on an initial day (D₀) administering to the subject the anti-BMP6 antibody or fragment; and
   (b) in a period of at least 3 consecutive weeks, said period starting on D₀, administering a plurality of doses of an ESA wherein blood Hb concentration in said subject is elevated from a baseline concentration on Do for the entire duration of said period,
   such that:—
   (i) for the entire duration of said period Hb concentration is no lower than 100% of baseline Hb concentration; and during said period Hb concentration reaches at least 120% of baseline; and/or
   (ii) for the entire duration of said period Hb concentration is increased over baseline by at least 1 g/dl.

In any Embodiment herein, in an example for the entire duration of said period Hb concentration is increased over baseline by at least 1 g/dl, eg, by at least 1.5, 2 or 2.5 g/dl. In an example, Hb concentration is no more than 11. 11.5 or 12 g/dl in the subject, eg, an adult male or female human.

Hb concentrations and MCH (see below) may be determined using one or more blood samples obtained from the subject. For example, as determined using a blood sample taken at the end of each week of said period (and the baseline determined using a sample taken at Do).
3. A combination of an amount of anti-BMP6 antibody or fragment and an amount of an ESA (eg, comprising multiple doses of said ESA) for use in a method of treating anaemia, wherein the antibody, fragment and method are according to Embodiment 2.
4. The combination of Embodiment 3, wherein the method comprises obtaining a single dose from said amount of antibody or fragment, wherein the single dose is administered to the subject on D₀, and obtaining a plurality of doses of said ESA, wherein at least one dose is administered weekly from D₀.

In any Embodiment herein, in an example the first dose of ESA is administered on D₀
5. The combination of Embodiment 3 or 4, wherein the antibody or fragment is comprised by a pharmaceutical composition, wherein the antibody or fragment is mixed with a dose of the ESA for administration to said subject on D₀.
6. A medical kit comprising the combination of any one of Embodiments 3 to 5, a first sterile container comprising said amount of antibody or fragment, and a second sterile container comprising said amount of ESA, and optionally instructions for carrying out said method.
7. An antagonist according to Embodiment 1, wherein the antagonist comprises or consists of an erythropoietin stimulating agent (ESA) (eg, comprised by multiple doses of said ESA), the method comprising
   (a) on an initial day (D₀) administering to the subject an anti-BMP6 antibody or fragment; and
   (b) in a period of at least 3 consecutive weeks, said period starting on D₀, administering a plurality of doses of said ESA wherein blood haemoglobin (Hb) concentration in said subject is elevated from a baseline concentration on Do for the entire duration of said period,
   such:—
   (i) for the entire duration of said period Hb concentration is no lower than 100% of baseline Hb concentration; and during said period Hb concentration reaches at least 120% of baseline; and/or
   (ii) for the entire duration of said period Hb concentration is increased over baseline by at least 1 g/dl.
8. The antagonist, combination or kit of any one of Embodiments 2 to 7, wherein
   (iii) the Hb concentration on the last day of said consecutive week period is at least 120% of the Hb concentration on the 7th day immediately preceding said last day.
9. The antagonist, combination or kit of any one of Embodiments 2 to 8, wherein ESA is administered to the subject within 24 hours of administration of the anti-BMP6 antibody or fragment.
10. The antagonist, combination or kit of any one of Embodiments 2 to 9, wherein said consecutive week period consists of a period of 3 or 4 consecutive weeks.
11. The antagonist, combination or kit of any one of Embodiments 2 to 10 (eg, Embodiment 10), wherein during said period Hb concentration reaches an increase in the range from 1 to 8 g/dl over baseline.

In any Embodiment herein, in an example during said period Hb concentration reaches an increase in the range from 1 to 3, 2.5, 2, 1.5 or 1.25 g/dl over baseline. For example the Hb concentration reaches an increase from 1 to 2 g/dl.
12. The antagonist, combination or kit of any one of Embodiments 2 to 11, wherein the period consists of 3 or 4 consecutive weeks and at the end of said period Hb concentration reaches at least 150% of baseline.
13. The antagonist, combination or kit of any one of Embodiments 2 to 12, wherein the period consists of 3 or 4 consecutive weeks and
   (a) for the entire duration of said period Hb concentration is no lower than 110% of baseline Hb concentration; and during said period Hb concentration reaches at least 150% of baseline; and/or
   (b) for the entire duration of said period Hb concentration is increased over baseline by at least 1 g/dl and during said period Hb concentration reaches an increase in the range from 1 to 8 g/dl over baseline. For example the Hb concentration reaches an increase from 1 to 2 g/dl.
14. The antagonist, combination or kit of any one of Embodiments 2 to 13, wherein in said period the antibody or fragment is administered on D₀.
15. The antagonist, combination or kit of Embodiment 14, wherein the antibody or fragment is administered as a single dose on D₀ to the subject.

In any Embodiment herein, in an example the antibody or fragment is administered as a single dose on Do to the subject, wherein the single dose is administered in one or a plurality of aliquots to the subject.
16. The antagonist, combination or kit of any one of Embodiments 2 to 15, wherein an initial ESA dose is administered on D₀ or no more than 2 days thereafter.
17. The antagonist, combination or kit of any one of Embodiments 2 to 16, wherein an ESA dose is administered on the 4-9^{th} (eg, on the 7^{th}) day immediately after D₀.
18. The antagonist, combination or kit of any one of Embodiments 2 to 17, wherein a ESA dose is administered on the 12-16^{th} (eg, on the14^{th}) day immediately after D₀.
19. The antagonist, combination or kit of any one of Embodiments 2 to 18, wherein a ESA dose is administered on the 19-23^{rd} (eg, on the 21^{st}) day immediately after Do.

In any Embodiment herein, in an example an ESA dose is administered in each of (i) the 4-9^{th} (eg, on the 7^{th}) day, (ii) the 12-16^{th} (eg, on the14^{th}) day and (iii) the 19-23^{rd} (eg, on the 21^{st}) day immediately after D₀.
20. The antagonist, combination or kit of any one of Embodiments 2 to 19, wherein an equivalent of 4 ESA doses is administered during said period.

By “equivalent” here, it is intended that a plurality of aliquots of the ESA can be administered (eg, on the same day or sequentially), wherein the aliquots amount to a total dose of the ESA. In an example, the ESA is darbepoetin alfa or Aranesp® and a dose is in the range from 15 to 100 mcg (micrograms); or from 30 to 100 mcg. In an example, the ESA is epoetin alfa and a dose is in the range from 3000 to 30000 units (ie, units refers to International Units, also known as IU, UI, IE, ME, NE in various languages).

Generally herein, a dose (eg, of antibody, fragment or ESA) can be administered in one aliquot or a plurality of aliquots (eg, on the same day, simultaneously, within a 30, 1 or 24 hour window).
21. The antagonist, combination or kit of any one of Embodiments 2 to 20, wherein ESA is administered to the subject during each of the first and second weeks after the initial ESA dose.
22. The antagonist, combination or kit of any one of Embodiments 2 to 21, wherein ESA is administered to the subject during each of the first, second and third weeks after the initial ESA dose.
23. The antagonist, combination or kit of Embodiment 21 or 22, wherein ESA is administered as a single dose at the end of each said week, optionally wherein said period consists of 3 or 4 weeks starting at D₀.
   In an example of any of the Embodiments, said period consists of 4 weeks starting at D₀ and anaemia is treated in the 4^{th} week.
24. The antagonist, combination or kit of any one of Embodiments 2 to 23, wherein no more than 4 doses of ESA are administered to the subject during said period and optionally a single dose of said antibody or fragment.
25. The antagonist, combination or kit of any one of Embodiments 2 to 24, wherein over said period (wherein the period is a 4 consecutive week period), the total dose of the antibody and total dose of ESA are administered to said subject in a ratio of X:Y, wherein X is from 10 to 2×10⁶ and Y=4.

In an example, a total weekly dose of ESA (eg, wherein the subject is a human) is from 10 or 15 to 80, 100, 200 or 300 mcg (micrograms). For example, the total weekly dose is from 10 to 80; from 15 to 80; or from 30 to 80 mcg. For example, the ESA comprises or consists of darbepoetin alfa, epoetin alfa or any other ESA disclosed herein. In an example, each dose of ESA (or a weekly dose) is administered to the subject in the range from 1.5 to 2 mcg/kg ESA.

In certain configurations, the method relates to reducing or sparing the administration of ESA. In this instance, for example, a total weekly dose of ESA (eg, wherein the subject is a human) is from 1 to 20 mcg, eg, from 1 up to 15 mcg. In an example where there is ESA sparing or reduction, each dose of ESA (or a weekly dose) is administered to the subject in the range from 0.01 or 0.1 to 0.3 or 1 mcg/kg ESA, eg, from 0.01 to 0.3; or from 0.1 to 0.3; or from 0.01 to 1; or from 0.1 to 1 mcg/kg.
26. The antagonist, combination or kit of any one of Embodiments 2 to 25, wherein Hb concentration at the end of said period (eg, a 3 or 4 consecutive week period) is at least 130% of Hb concentration in a control anaemia patient of the same species that has received administration of the anti-BMP6 antibody or fragment in the same dosing regimen as said subject except the patient has not received administration of an ESA during said period.
27. The antagonist, combination or kit of Embodiment 26, wherein said Hb concentration at the end of said period is significantly higher than in said control at the end of said period, as determined by a p-value of p<0.0001.
28. The antagonist, combination or kit of any one of Embodiments 2 to 27, wherein mean corpuscular haemoglobin (MCH) at the end of said period is at least 109% of MCH in a control anaemia patient of the same species that has received administration of the anti-BMP6 antibody or fragment in the same dosing regimen as said subject except the patient has not received administration of an ESA during said period.

The mean corpuscular haemoglobin (MCH) is the average mass of haemoglobin per red blood cell in a sample of blood.
29. The antagonist, combination or kit of any one of Embodiments 2 to 28, wherein Hb concentration at the end of said period (eg, a 3 or 4 consecutive week period) is at least 120% of Hb concentration in a control anaemia patient of the same species that has received administration of said ESA in the same dosing regimen as said subject except the patient has not received administration of an anti-BMP6 antibody or fragment during said period. Optionally, the control patient has received a control IgG4 antibody that does not specifically bind BMP6 (eg, wherein the BMP6 antibody and the control antibody are administered to the subject and control patient respectively in the same dose). Optionally, X is from 10 to 2×10⁵, 2×10⁴ or 2×10³.
30. The antagonist, combination or kit of Embodiment 29, wherein said Hb concentration at the end of said period is significantly higher than in said control at the end of said period, as determined by a p-value of p<0.0001.
31. The antagonist, combination or kit of any one of Embodiments 2 to 30, wherein mean corpuscular haemoglobin (MCH) at the end of said period (eg, a 3 or 4 consecutive week period) is at least 119% of MCH in a control anaemia patient of the same species that has received administration of said ESA in the same dosing regimen as said subject except the patient has not received administration of an anti-BMP6 antibody or fragment during said period.
32. The antagonist, combination or kit of Embodiment 31, wherein said MCH at the end of said period is significantly higher than in said control at the end of said period, as determined by a p-value of p<0.0001.
33. The antagonist, combination or kit of any one of Embodiments 2 to 32, wherein said subject at D₀ suffers from anaemia of chronic disease (ACD) and optionally wherein the anaemia is associated with chronic inflammation (eg, the subject suffers from arthritis) or a bacterial infection (eg, Streptococcus infection), or wherein the subject is a chronic kidney disease (CKD) patient.
34. The antagonist, combination or kit of any one of Embodiments 2 to 33, wherein the anaemia in said subject at the end of said period is less severe than on D₀.
35. The antagonist of Embodiment 1, wherein the antagonist comprises or consists of an anti-BMP6 antibody or fragment.
36. The antagonist, combination or kit of any one of Embodiments 2 to 34, wherein the antibody or fragment competes with a reference antibody for binding BMP6, wherein the reference antibody is mAb507 (R&D Systems) or an antibody comprising
   a. Heavy chains each comprising or consisting of the amino acid sequence of SEQ ID NO: 1 or 2, and light chains each comprising or consisting of the amino acid sequence of SEQ ID NO: 3; or
   b. Heavy chains each comprising or consisting of the amino acid sequence of SEQ ID NO: 4 and light chains each comprising or consisting of the amino acid sequence of SEQ ID NO: 5.

Competition herein can, for example, be determined by SPR (eg, at 37 degrees cat pH7.6 and optionally as a Fab); by ELISA; by fluorescence activated cell sorting (FACS); or in a homogenous time resolved fluorescence (HTRF) assay. SPR may be carried out using Biacore™, Proteon™ or another standard SPR technique. In one embodiment, competition is determined by ForteBio Octet® Bio-Layer Interferometry (BLI) such techniques being readily apparent to the skilled person.

In an alternative, the reference antibody is any anti-BMP6 antibody disclosed in WO2016098079 (the sequences and disclosure relating to such antibodies being incorporated herein for potential use in the present invention).
37. The antagonist, combination or kit of any one of Embodiments 2 to 36, wherein the antibody or fragment competes with said reference antibody for binding to SEQ ID NO: 6. Said SEQ ID NO: 6 can be used as a peptide per se, part of a larger peptide or part of a BMP6 protein (eg, a wild type human BMP6 or recombinantly produced BMP6).

Additionally or alternatively, the antibody or fragment competes with said reference antibody for binding to a further sequence selected from the group consisting of SEQ ID NOs: 7-19. Said further sequence can be used as a peptide per se, part of a larger peptide (eg, comprising SEQ ID NO: 6) or part of a BMP6 protein (eg, a wild type human BMP6 or recombinantly produced BMP6, eg, comprising SEQ ID NO: 6). For example, the antibody or fragment competes with said reference antibody for binding to SEQ ID NO: 7. For example, the antibody or fragment competes with said reference antibody for binding to SEQ ID NO: 8. For example, the antibody or fragment competes with said reference antibody for binding to SEQ ID NO: 9. For example, the antibody or fragment competes with said reference antibody for binding to SEQ ID NO: 10. For example, the antibody or fragment competes with said reference antibody for binding to SEQ ID NO: 11. For example, the antibody or fragment competes with said reference antibody for binding to SEQ ID NO: 12. For example, the antibody or fragment competes with said reference antibody for binding to SEQ ID NO: 13. For example, the antibody or fragment competes with said reference antibody for binding to SEQ ID NO: 14. For example, the antibody or fragment competes with said reference antibody for binding to SEQ ID NO: 15. For example, the antibody or fragment competes with said reference antibody for binding to SEQ ID NO: 16. For example, the antibody or fragment competes with said reference antibody for binding to SEQ ID NO: 17. For example, the antibody or fragment competes with said reference antibody for binding to SEQ ID NO: 18. For example, the antibody or fragment competes with said reference antibody for binding to SEQ ID NO: 19. The sequence ID numbers in this paragraph and for Embodiments herein are those disclosed in WO2017191437, the disclosure of which is incorporated herein.
38. The antagonist, combination or kit of any one of Embodiments 2 to 37, wherein the antibody or fragment competitively inhibits the binding of soluble haemojuvelin (HJV) to BMP6.
39. The antagonist, combination or kit of any one of Embodiments 1 to 38, wherein the antibody or fragment does not competitively inhibit the binding of soluble haemojuvelin (HJV) to BMP6 (eg, as determined by SPR, HTRF or ELISA).
40. The antagonist, combination or kit of any one of Embodiments 2 to 39, wherein the antibody comprises VH domains encoded by a VDJ region sequence, wherein the VDJ is derived from the recombination of a VH gene segment, D gene segment and JH gene segment, wherein the VH is a human germline (i) VH1-3, (ii) VH2-5 or (iii) VH3-15 gene segment.
41. The antagonist, combination or kit of any one of Embodiments 2 to 40, wherein the antibody comprises VL domains encoded by a VJ region sequence, wherein the VJ is derived from the recombination of a VL gene segment and JL gene segment, wherein the VL is a human germline (iv) Vκ3-20, (v) Vλ3-1, (vi) Vκ1-17 or (vii) Vλ1-40.
42. The antagonist, combination or kit of any one of Embodiments 2 to 41, wherein the antibody or fragment binds to BMP6 with a stronger affinity (lower KD determined by SPR) than binding to BMP7; and optionally binds to BMP6 with a stronger affinity than to BMP5.

Optionally the antibody or fragment binds to BMP6 with a stronger affinity than to each of BMP2, 4, 5 and 9.
43. The antagonist, combination or kit of any one of Embodiments 2 to 42, wherein the antibody or fragment binds to a human BMP6 sequence comprising SEQ ID NO: 6.
44. The antagonist, combination or kit of any one of Embodiments 2 to 43, wherein the antibody has an affinity (KD) for binding BMP6 of from 1 pM to 5 nM, optionally wherein binding is determined by SPR using a Fab of said antibody at 37 °C at pH 7.6.

In an example, the antibody (eg, as a Fab) or fragment has an affinity (KD) for binding BMP6 of
(a) from 2, 3, 4, 5 or 10 pM to 3, 4 or 5 nM;
(b) from 1-10 pM to 5 nM;
(c) from 10 pM to 3, 4 or 5 nM;
(d) from 50 or 80 pM to 200 nM;
(e) from 50 or 80 pM to 150 nM; or
(f) from 50 or 80 pM to 100 nM.

In an example, the KD is (or is about) 5-15 pM (eg, 10 pM). In an example, the KD is (or is about) 2-5 nM (eg, 3 nM). In an example, the KD is (or is about) 100-400 pM (eg, 140 or 390 pM).
45. The antagonist, combination or kit of any one of Embodiments 2 to 44 (eg, Embodiment 44), wherein the antibody has off-rate (K_{off}) for binding BMP6 of from 1×10⁻⁵ to 1×10⁻³ S⁻¹, optionally wherein binding is determined by SPR using a Fab of said antibody at 37 °C at pH 7.6.

In an example, the antibody (eg, as a Fab) or fragment has an off-rate (K_{off}) for binding BMP6 of
(a) from 1×10⁻⁵ to 5×10⁻⁴ S⁻¹;
(b) from 1×10⁻⁵ to 6×10⁻⁴ S⁻¹;
(c) from 1×10⁻⁵ to 7×10⁻⁴ S⁻¹;
(d) from 1×10⁻⁵ to 8×10⁻⁴ S⁻¹;
(e) from 2×10⁻⁵ to 1×10⁻³ S⁻¹;
(f) from 2×10⁻⁵ to 5×10⁻⁴ S⁻¹;
(g) from 2×10⁻⁵ to 6×10⁻⁴ S⁻¹;
(h) from 2×10⁻⁵ to 7×10⁻⁴ S⁻¹; or
(i) from 2×10⁻⁵ to 8×10⁻⁴ S⁻¹.

In an example, the K_{off} is (or is about) 5×10⁻⁴ S⁻¹ (eg, when the KD is (or is about) from 2 nM to 400 pM; when the KD is (or is about) 2-5 nM (eg, 3 nM); or when the KD is (or is about) 100-400 pM (eg, 140 or 390 pM)). In an example, the K_{off} is (or is about) 3×10⁻⁵ S⁻¹ (eg, when the KD is (or is about) from 5-15 pM (eg, 10 pM)).
46. The antagonist, combination or kit of any one of Embodiments 2 to 45 (eg, Embodiment 44 and/or 45), wherein the antibody has on-rate (Kₒₙ) for binding BMP6 of from 1×10⁵ to 1×10⁷ M⁻¹S⁻¹, optionally wherein binding is determined by SPR using a Fab of said antibody at 37 °C at pH 7.6.

In an example, the antibody (eg, as a Fab) or fragment has an on-rate (Kₒₙ) for binding BMP6 of
(a) from 1×10⁵ to 1×10⁶ M⁻¹S⁻¹;
(b) from 1×10⁵ to 2×10⁶ M⁻¹S⁻¹;
(c) from 1×10⁵ to 3×10⁶ M⁻¹S⁻¹;
(d) from 1×10⁵ to 4×10⁶ M⁻¹S⁻¹;
(e) from 1×10⁵ to 5×10⁶ M⁻¹S⁻¹;
(f) from 2×10⁵ to 5×10⁶ M⁻¹S⁻¹;
(g) from 3×10⁵ to 5×10⁶ M⁻¹S⁻¹;
(h) from 4×10⁵ to 5×10⁶ M⁻¹S⁻¹;
(i) from 5×10⁵ to 5×10⁶ M⁻¹S⁻¹; or
(j) from 6×10⁵ to 5×10⁶ M⁻¹S⁻¹.

In an example, the Kₒₙ is (or is about) 1 or 2×10⁻⁵ M⁻¹S⁻¹ (eg, when the KD is 2-5 nM (eg, 3 nM)). In an example, the Kₒₙ is (or is about) 1-4, 1, 2, 3 or 4×10⁻⁶ M⁻¹S⁻¹ (eg, when the KD is (or is about) from 5-400 pM (eg, 140 or 390 pM) or 5-15 pM (eg, 10 pM)).
47. The antagonist, combination or kit of any one of Embodiments 2 to 46, wherein
   (a) the period consists of 3 or 4 consecutive weeks and
      (i) for the entire duration of said period Hb concentration is no lower than 110% of baseline Hb concentration; and during said period Hb concentration reaches at least 120% of baseline; and/or
      (ii) for the entire duration of said period Hb concentration is increased over baseline by at least 1 g/dl and during said period Hb concentration reaches an increase in the range from 1 to 8 g/dl over baseline;
   (b) wherein a dose of ESA is administered at least twice during the first two or three weeks of said period;
   (c) wherein the antibody or fragment binds to BMP6 with a stronger affinity (lower KD determined by SPR) than binding to BMP7; and optionally binds to BMP6 with a stronger affinity than to BMP5 (and optionally binds to BMP6 with a stronger affinity than to each of BMP2, 4, 5 and 9); and
   (d) wherein the antibody or fragment competes with a reference antibody for binding BMP6, wherein the reference antibody is mAb507 (R&D Systems) or an antibody comprising
      I. Heavy chains each comprising or consisting of the amino acid sequence of SEQ ID NO: 1 or 2, and light chains each comprising or consisting of the amino acid sequence of SEQ ID NO: 3; or
      II. Heavy chains each comprising or consisting of the amino acid sequence of SEQ ID NO: 4 and light chains each comprising or consisting of the amino acid sequence of SEQ ID NO: 5.

Optionally, in part I the heavy chains consist of the amino acid sequence of SEQ ID NO: 1 and the light chains consist of the amino acid sequence of SEQ ID NO: 3. Optionally, in part I the heavy chains consist of the amino acid sequence of SEQ ID NO: 2 and the light chains consist of the amino acid sequence of SEQ ID NO: 3. Optionally, in part II the heavy chains consist of the amino acid sequence of SEQ ID NO: 4 and the light chains consist of the amino acid sequence of SEQ ID NO: 5.

In an example (as per the antibody used in Example 2 below), in part (d) the anti-BMP6 antibody of the invention is an antibody that competes with a reference antibody of part I or part II in an HTRF assay. For example, wherein in the HTRF assay the antibody of the invention is a labelled antibody that is pre-incubated with human BMP6 and subsequently combined with unlabelled reference antibody (according to part I or II), wherein competition between the antibodies is detected by the assay. In an example, the assay uses AlexaFluor™ 647 labelled antibody of the invention. In an alternative, the human BMP6 is labelled (eg, with AlexaFluor™ 647, the test antibody is labelled with biotin for binding to Eu3+cryptate-streptavidin, and the reference antibody is unlabelled).

Optionally, the anti-BMP6 antibody of the invention (test antibody) competes in an HTRF assay with the reference antibody for binding human BMP6 (or binds the same epitope of human BMP6 as the reference antibody), wherein the assay uses a directly or indirectly labelled test antibody directly or indirectly labelled with a donor (such as for example Eu3+cryptate) or an acceptor fluorophore (such as for example AlexaFluor™ 647) and a target BMP6 labelled with either a donor or acceptor fluorophore to enable energy transfer between donor and acceptor, whereby a fluorescence signal is produced and detected. In an example, where AlexaFluor™ 647 labelling is used, competition is detected by a reduction in fluorescence signal at 665 nM of at least 20% when the test antibody is in the presence of the reference antibody versus signal without the reference antibody. Optionally, the reduction in signal at 665 nM is at least 20, 30, 40, 50, 60, 70, 80 or 90%.

Optionally, the anti-BMP6 antibody (test antibody) is one that competes in a HTRF assay with a reference antibody for binding human BMP6 (or binds the same epitope of human BMP6 as the reference antibody), wherein the reference antibody comprises heavy chains each comprising the amino acid sequence of SEQ ID NO: 1 or 2, and light chains each comprising the amino acid sequence of SEQ ID NO: 3, wherein the assay uses the test antibody directly or indirectly labelled with a donor label (such as for example Eu3+cryptate) or an acceptor fluorophore label (such as for example AlexaFluor™ 647) and a human BMP6 labelled with either an acceptor fluorophore or donor respectively to enable energy transfer between donor and acceptor, wherein said competition between the antibodies is detected by a reduction in fluorescence signal of at least 20% when the test antibody is in the presence of the reference antibody versus signal without the reference antibody. For example, the test antibody is directly or indirectly labelled with AlexaFluor™ 647 and competition is detected by a reduction in fluorescence signal at 665 nM of at least 20% when the test antibody is in the presence of the reference antibody versus signal without the reference antibody. Optionally, the reduction in signal at 665 nM is at least 20, 30, 40, 50, 60, 70, 80 or 90%.

Optionally, the anti-BMP6 antibody (test antibody) also competes in an HTRF assay with a reference antibody for binding human BMP6 (or binds the same epitope of human BMP6 as the reference antibody), wherein the reference antibody comprises heavy chains each comprising the amino acid sequence of SEQ ID NO: 4, and light chains each comprising the amino acid sequence of SEQ ID NO: 5, wherein the assay for example uses the test antibody directly or indirectly labelled with a donor label (such as for example Eu3+cryptate) or an acceptor fluorophore label (such as for example Alexa Fluor™ 647) and a human BMP6 labelled with either an acceptor fluorophore or donor respectively to enable energy transfer between donor and acceptor, wherein said competition between the antibodies is detected by a reduction in fluorescence signal of at least 20% when the test antibody is in the presence of the reference antibody versus signal without the reference antibody. For example, the test antibody is directly or indirectly labelled with AlexaFluor™ 647 and competition is detected by a reduction in fluorescence signal at 665 nM of at least 20% when the test antibody is in the presence of the reference antibody versus signal without the reference antibody. Optionally, the reduction in signal at 665 nM is at least 20, 30, 40, 50, 60, 70, 80 or 90%.

For example in part (b) a dose of ESA is administered 2, 3 or 4 times during the first 3 weeks of said period, or during said period.

In an alternative, the reference antibody is any anti-BMP6 antibody disclosed in WO2016098079 (the sequences and disclosure relating to such antibodies being incorporated herein for potential use in the present invention).
48. The antagonist, combination or kit of any one of Embodiments 1 to 47 for
   a. treating ACD in the subject;
   b. treating or preventing moderate or severe anaemia in the subject;
   c. treating or preventing anaemia in the subject, wherein the subject suffers from an inflammatory disease or condition;
   d. eliminating or reducing the need to administer iron or blood transfusion to the subject;
   e. treating or preventing anaemia in the subject, wherein the subject suffers from a microbial infection; or
   f. reducing administration of ESA to the subject.
49. A method of treating anaemia in a subject, the method comprising
   (a) on an initial day (Do) administering to the subject an anti-BMP6 antibody or fragment; and
   (b) in a period of at least 3 consecutive weeks, said period starting on Do, administering a plurality of doses of an erythropoietin stimulating agent (ESA) wherein blood haemoglobin (Hb) concentration in said subject is elevated from a baseline concentration on Do for the entire duration of said period,
   such that for the entire duration of said period:—
   (i) Hb concentration is no lower than 100% of baseline Hb concentration; and during said period Hb concentration reaches at least 120% of baseline; and/or
   (ii) Hb concentration is increased over baseline by at least 1 g/dl.
50. The method of Embodiment 49, wherein the method, antibody fragment or ESA is as recited in any one of Embodiments 2 to 48.
51. The antagonist, combination, kit or method of any preceding Embodiment, wherein the anaemia is moderate or severe anaemia.
52. An anti-Bone Morphogenetic Protein 6 (BMP6) antagonist for use in a method of maintaining a blood haemoglobin (Hb) level of at least 10 g/dL in a subject, the method comprising administering an anti-BMP6 antagonist and an erythropoiesis stimulating agent (ESA) to the subject.

Any anti-BMP6 antibody or fragment of the invention can be used as the anti-Bone Morphogenetic Protein 6 (BMP6) antagonist.
53. An anti-Bone Morphogenetic Protein 6 (BMP6) antagonist for use in a method of preventing the blood haemoglobin level of a subject from decreasing to less than 10 g/dl, the method comprising administering an anti-BMP6 antagonist and an erythropoiesis stimulating agent (ESA) to the subject.
54. An anti-Bone Morphogenetic Protein 6 (BMP6) antagonist for use in a method of raising blood haemoglobin to a level of at least 10 g/dL in a subject suffering from anaemia, the method comprising administering an anti-BMP6 antagonist and an erythropoiesis stimulating agent (ESA) to the subject, wherein said anaemia is treated.
55. An anti-Bone Morphogenetic Protein 6 (BMP6) antagonist for use in a method of treating or preventing anaemia in a subject suffering from an inflammatory disease or condition, the method comprising administering an anti-BMP6 antagonist and an erythropoiesis stimulating agent (ESA) to the subject, wherein said anaemia is treated or prevented.
56. An anti-Bone Morphogenetic Protein 6 (BMP6) antagonist for use in a method of eliminating or reducing the need to administer iron or blood transfusion to a subject suffering from anaemia, the method comprising administering an anti-BMP6 antagonist and an erythropoiesis stimulating agent (ESA) to the subject, wherein said need is eliminated or reduced.
57. An anti-Bone Morphogenetic Protein 6 (BMP6) antagonist for use in a method of treating or preventing anaemia in a subject suffering from a microbial infection, the method comprising administering an anti-BMP6 antagonist and an erythropoiesis stimulating agent (ESA) to the subject.
58. An anti-Bone Morphogenetic Protein 6 (BMP6) antagonist for use in a method of reducing administration of an erythropoiesis stimulating agent (ESA) to a subject suffering from anaemia for treating anaemia, the method comprising administering an anti-BMP6 antagonist and said ESA, wherein anaemia is treated in the subject.
59. An anti-Bone Morphogenetic Protein 6 (BMP6) antagonist for use in a method of treating or reducing the risk of anaemia in a subject suffering from or at risk of anaemia, the method comprising administering an anti-BMP6 antagonist and a low dose of an erythropoiesis stimulating agent (ESA) to the subject, wherein anaemia is treated or the risk of anaemia is reduced in the subject.
60. The antagonist of any one of Embodiments 52 to 59, wherein the antagonist is according to any one of Embodiments 1 to 1, 2, 7 to 48 and 51.
61. The antagonist of any one of Embodiments 52 to 59, wherein the antagonist is according to any other of Embodiments 52 to 59.
62. The antagonist, combination, kit or method of any preceding Embodiment, wherein the ESA is
   a. Epoetin alfa and is administered at a weekly dose of less than 1000, 1500, 2500, 5000, 11000, 18000, 34000 or 90000 units, optionally wherein the subject has previously received a weekly epoetin alfa treatment of <1500, 1500 to 2499, 2500 to 4999, 5000 to 10999, 11000 to 17999, 18000 to 33999, 34000 to 89999 or ≥90000 units respectively;
   b. darbepoetin alfa or Aranesp® and is administered at a weekly dose of less than 6.25, 10, 12.5, 20, 25, 40, 60, 100 or 200 mcg, optionally wherein the subject has previously received a weekly darbepoetin alfa or Aranesp® treatment of 6.25, 10, 12.5, 20, 25, 40, 60, 100 or 200 mcg respectively; or
   c. darbepoetin alfa or Aranesp® and is administered at a weekly dose of less than 6.25, 10, 20, 40, 60, 100 or 200 mcg, optionally wherein the subject has previously received a weekly Epoetin alfa treatment of 1500 to 2499, 2500 to 4999, 5000 to 10999, 11000 to 17999, 18000 to 33999, 34000 to 89999 or ≥90,000 units respectively.
63. The antagonist, combination, kit or method of any preceding Embodiment, wherein the anti-BMP6 antagonist is an antibody and each dose is administered at a total of no more than 30 mg/kg.
64. An anti-BMP6 antagonist and/or an ESA for use in a therapeutic regimen method for treating or preventing anaemia in a subject suffering from or at risk of anaemia, the regimen comprising simultaneously or sequentially administering an anti-BMP6 antagonist and an ESA to the subject, wherein
   a. On day zero the antagonist is administered to the subject; and no later than day 7 (eg, on day 1) the ESA is administered to the subject; or
   b. On day zero the ESA is administered to the subject; and no later than day 7 (eg, on day 1) the antagonist is administered to the subject; or
   c. On day zero the antagonist and the ESA are simultaneously administered to the subject; or
   d. On day zero the subject has already received the ESA and on day zero the antagonist is administered to the subject; or
   e. On day zero the subject has already received the antagonist and on day zero the ESA is administered to the subject;
      whereby at day 14 or later the blood haemoglobin level is at least 10 g/dL in the subject,
      wherein said anaemia is treated or prevented.
65. The antagonist and/or ESA according to Embodiment 64, which is further according to any one of Embodiments 1 to 1, 2, 7 to 48 and 51.
66. The antagonist and/or ESA according to Embodiment 64 or 65, wherein the anti-BMP6 antagonist and ESA are administered to the subject no more than 7 days apart.
67. The antagonist and/or ESA according to Embodiment 64, 65 or 66, wherein the regimen maintains blood Hb level in the subject at more than 10 g/dL in the subject.
68. The antagonist and/or ESA according to any one of Embodiments 64 to 67, wherein the method maintains or raises blood haemoglobin level to at least 10 g/dL in the subject at least 13 or 14 days after the subject has received the anti-BMP6 antagonist and ESA.
69. The antagonist and/or ESA according to Embodiment 67 or 68, wherein the anti-BMP6 antagonist and ESA are administered to the subject no more than 1 day apart.
70. The antagonist and/or ESA according to any one of Embodiments 64 to 69, wherein the anti-BMP6 antagonist and ESA are administered to the subject simultaneously.
71. The antagonist and/or ESA according to any one of Embodiments 64 to 70, wherein the blood haemoglobin level of the subject is prevented from decreasing to less than 10 g/dL (eg, at day 14).
72. The antagonist and/or ESA according to any one of Embodiments 64 to 71, wherein the blood haemoglobin of the subject is raised to a level of at least 10 g/dL (eg, at day 14).
73. The antagonist and/or ESA according to any one of Embodiments 64 to 72, wherein moderate or severe anaemia is prevented in the subject (eg, at day 14).
74. The antagonist, combination, kit, ESA or method of any preceding Embodiment, wherein the subject is suffering from
   a. an inflammatory disease or condition; or
   b. an infection;
   c. kidney disease;
   d. HIV or undergoing HIV treatment; or
   e. cancer; and
      anaemia is treated or prevented in the subject.
75. The antagonist, combination, kit, ESA or method of any preceding Embodiment, wherein the subject is a mammal.
76. The antagonist, combination, kit, ESA or method of any preceding Embodiment, in combination with an anti-inflammatory agent, or wherein an anti-inflammatory agent is administered to the subject.
77. The antagonist, combination, kit, ESA or method of any preceding Embodiment, wherein the ESA is an erythropoietin.

In an example, the subject is suffering from chronic kidney disease (CKD). Reference is made to “KDIGO Clinical Practice Guideline for Anaemia in Chronic Kidney Disease”, Kidney International Supplements (2012) 2, 279; doi: 10.1038/kisup.2012.37. This discusses stages of chronic kidney disease (stages 1-5), diagnosis, CKD nomenclature, Hb levels and ranges in humans of various ages and ESA hyporesponsiveness. This reference discloses:—

### Diagnosis of Anaemia

Diagnose anaemia in adults and children >15 years with CKD when the Hb concentration is <13.0 g/dl in males and <12.0 g/dl in females. (Not Graded)
Diagnose anaemia in children with CKD if Hb concentration is <11.0 g/dl in children 0.5-5 years, <11.5 g/dl in children 5-12 years, and <12.0 g/dl in children 12-15 years. (Not Graded)

Thus, in the present invention, optionally
(a) the subject is an adult or child >15 years with CKD and during said period (eg, at the beginning of the 3^{rd} week from D₀) Hb concentration <13.0 g/dl (when the subject is male) or <12.0 g/dl (when the subject is female); or
(b) the subject with CKD and during said period (eg, at the beginning of the 3^{rd} week from Do) Hb concentration <11.0 g/dl (wherein the subject is aged 0.5-5 years), <11.5 g/dl (wherein the subject is aged 5-12 years), or <12.0 g/dl (wherein the subject is aged 12-15 years).

Optionally, the subject is a CKD patient that has a diagnosed malignancy, has suffered one or more strokes, and/or has suffered a malignancy. ESA therapy is usually to be proceeded with caution (if at all) in such patients, and thus the invention (especially ESA reducing or sparing aspects thereof) are advantageous in such subjects.

Optionally, the subject is a CKD 5D patient (eg, an human adult, eg, a male or female) with Hb concentration from 9.0 to 10.0 g/dl.

Optionally, the invention is for maintaining Hb concentration above 11.5 g/dl in an human adult patient with CKD.

Optionally, the invention is for maintaining Hb concentration from 9.0 to 13 g/dl (eg, 9.0 to 11.5 g/dl) in an adult human patient with CKD.

Optionally, the invention is for maintaining Hb concentration from 11.0 to 12 g/dl in a paediatric human patient with CKD. In an example, the patient is 15 or younger; or younger than 15; or 10 or younger.

In an example, the CKD patient is an adult male. In another example, the CKD patient is an adult female.

Optionally, the subject (eg, an adult human) is a CKD 5HD patient, a patient on hemofiltration or a patient on hemodiafiltration therapy, wherein the method comprises intravenous or subcutaneous administration of ESA.

Optionally, the subject (eg, an adult human) is a CKD ND or CKD 5PD patient, wherein the method comprises subcutaneous administration of ESA.

Optionally, before administration of the anti-BMP6 antibody of fragment, the patient is ESA hporesponsive indicated by less than 5% increase or no increase in Hb concentration after a month ESA treatment (prior to carrying out the method of the invention).

Reference is made to WO2017191437 for general methodology and tests (eg, see the Examples therein).

### Inhibiting HJV-Independent BMP Receptor Dimerisation & Signalling

The art has studied signalling involving BMP6 complexed with HJV. Latour et al, however, proposed an alternative pathway in which BMP6-mediated BMP receptor dimerisation occurs without complexing HJV with BMP6 (Hepatology. 2016 January; 63(1):126-37. doi: 10.1002/hep.28254. Epub 2015 Nov. 12, “Differing impact of the deletion of hemochromatosis-associated molecules HFE and transferrin receptor-2 on the iron phenotype of mice lacking bone morphogenetic protein 6 or hemojuvelin”). As shown in Example 8, the inventors surprisingly found that they could inhibit HJV-independent dimerisation of BMP receptors in human cells by BMP6 using anti-BMP6 antagonists, such as antibodies (eg, in IgG4 format). Thus, in one configuration, the invention relates to inhibiting formation of BMP-BMPR complexes devoid of HJV, or intracellular signalling triggered by such complexes, for the treatment or prevention of a hepcidin-mediated disease or condition in a human or animal subject.

For example, the following Concepts are provided:—
1. An antibody or fragment that specifically binds to a bone morphogenetic protein (BMP) for use in a method of treating or preventing a disease or condition caused by haemojuvelin (HJV)-deficient BMP-BMP receptor (BMPR) complexes in a human or animal subject, wherein the method comprises administering the antibody or fragment to the subject for inhibiting formation of said complexes and/or inhbiting triggering of intracellular signalling by such complexes in the subject, whereby a HJV-independent BMP-BMPR mediated disease or condition is treated or prevented.
2. An antibody or fragment that specifically binds to a bone morphogenetic protein (BMP) for use in a method of treating or preventing HJV-independent anaemia or osteoporosis in a human or animal subject, wherein the method comprises administering the antibody or fragment to the subject for inhibiting formation of haemojuvelin (HJV)-deficient BMP-BMP receptor (BMPR) complexes and/or inhbiting triggering of intracellular signalling by such complexes in the subject, whereby HJV-independent anaemia or osteoporosis is treated or prevented.
3. An antibody or fragment that specifically binds to a bone morphogenetic protein (BMP) for use in a method of treating or preventing haemojuvelin (HJV)-independent anaemia or osteoporosis in a human or animal subject, wherein the method comprises administering the antibody to the subject, whereby HJV-independent anaemia or osteoporosis is treated or prevented.
4. An antibody or fragment for treating or preventing a disease or condition in a human or non-human animal subject, wherein
   a. the disease or condition is mediated by bone morphogenetic protein (BMP) receptor multimerization in hepatic or bone cells of the subject; and
   b. the antibody or fragment is administered to said subject for inhibiting said receptor multimerization, whereby the disease or condition is treated or prevented.
5. The antibody or fragment of any preceding Concept, wherein the BMP is BMP2, 4, 5, 6, 7, or 9.
6. The antibody or fragment of any preceding Concept, wherein the BMP is BMP6.
7. The antibody or fragment of any preceding Concept, wherein the BMP and receptor are human BMP and BMPR.
8. The antibody or fragment of any preceding Concept, wherein the antibody is an anti-BMP6 selected from (i) mAb507, (ii) an antibody comprising VH domains wherein each domain comprises the amino acid sequence of SEQ ID NO: 402; and VL domains, wherein each domain comprises the amino acid sequence of SEQ ID NO: 410; (iii) an antibody comprising VH domains wherein each domain comprises the amino acid sequence of SEQ ID NO: 418; and VL domains, wherein each domain comprises the amino acid sequence of SEQ ID NO: 426, and (iv) an antibody comprising VH domains wherein each domain comprises the amino acid sequence of SEQ ID NO: 114; and VL domains, wherein each domain comprises the amino acid sequence of SEQ ID NO: 123; or wherein the antibody or fragment competes (as determined by SPR) for binding to human BMP6 with an antibody selected from (i) to (iv).
9. The antibody or fragment of any preceding Concept, wherein the antibody or fragment is in an IgG4 (eg, IgG4PE) format.
10. The antibody or fragment of any preceding Concept, wherein said multimerization or complex formation is multimerization of (i) a Type I BMP receptor with (ii) a Type II BMP receptor.
11. The antibody or fragment of Concept 10, wherein said Type I BMP receptor is SKR1, CD292 or CDw293.
12. The antibody or fragment of any preceding Concept, wherein said subject is a human whose genome comprises a SKR1 nucleotide sequence comprising SNP rs13406336 and/or rs188547477; or wherein the human expresses a SKR1 with alanine at position 15 and/or an arginine at position 160; wherein said multimerization is multimerization of (iii) said SKR1 or a SKR1 encoded by said nucleotide sequence with (iv) a BMP receptor.
13. The antibody or fragment of any preceding Concept, wherein said subject is a human whose genome comprises a CD292 nucleotide sequence comprising one, two or three SNPs selected from rs11528010, rs142454490 and rs35619497; or wherein the human expresses a CD292 comprising one, two or three amino acids selected from a proline at position 2, a threonine at position 33 and an arginine at position 443; wherein said multimerization is multimerization of (v) said CD292 or a CD292 encoded by said nucleotide sequence with (vi) a BMP receptor.
14. The antibody or fragment of any preceding Concept, wherein said subject is a human whose genome comprises a CDw293 nucleotide sequence comprising one, two or three SNPs selected from rs34231464, rs138801821, rs200035802, rs143554488, rs35973133 and rs112111860; or wherein the human expresses a CDw293 comprising one, two or three amino acids selected from an arginine at position 149, a valine at position 140, an arginine at position 31, a serine at position 175, an arginine at position 224 and an aspartate at position 297; wherein said multimerization is multimerization of (vii) said CDw293 or a CDw293 encoded by said nucleotide sequence with (viii) a BMP receptor.
15. The antibody or fragment of any one of Concepts 10 to 14, wherein said Type II BMP receptor or the receptor of (iv), (vi) or (viii) is BRK-3, ACVR2A or ACVR2B.
16. The antibody or fragment of any preceding Concept, wherein said subject is a human whose genome comprises a BRK-3 nucleotide sequence comprising one, two or three SNPs selected from rs2228545, rs112862820, rs140683387 and rs201067849; or wherein the human expresses a BRK-3 comprising one, two or three amino acids selected from a serine at position 775, an asparagine at position 29, a glutamine at position 31 and a valine at position 348; wherein said multimerization is multimerization of (ix) said BRK-3 or a BRK-3 encoded by said nucleotide sequence with (x) a BMP receptor.
   The antibody or fragment of any preceding Concept, wherein said subject is a human whose genome comprises a ACVR2B nucleotide sequence comprising SNPB rs121434437; or wherein the human expresses ACVR2B comprising an arginine at position 40; wherein said multimerization is multimerization of (xi) said ACVR2B or a ACVR2B encoded by said nucleotide sequence with (xii) a BMP receptor.
17. The antibody or fragment of any preceding Concept, wherein a receptor or each receptor is a BMP6 receptor, BMP7 receptor, BMP2 receptor or BMP9 receptor.
18. The antibody or fragment of any preceding Concept, for inhibiting signalling in hepatic or bone cells of said subject, wherein the signalling is mediated by said receptor multimerization.
19. The antibody or fragment of any preceding Concept, wherein the signalling is Smad signalling.
20. The antibody or fragment of any preceding Concept, for inhibiting elevation of Smad mRNA in hepatic cells of said subject, whereby the disease or condition is treated or prevented.
21. A method of treating or preventing a disease or condition in a human or non-human animal subject, wherein
   a. the disease or condition is mediated by HJV-independent bone morphogenetic protein (BMP) receptor multimerization in hepatic or bone cells of the subject; and
   b. the method comprises administering an anti-BMP antibody or fragment to said subject for inhibiting said receptor multimerization, whereby the disease or condition is treated or prevented.
22. The method of Concept 22, wherein the antibody, fragment and/or receptor(s) are according to any one of Concepts 1 to 21.

### ESA Sparing Aspects & Treating ESA-Refractory or Low Responder Subjects

In an example, the antibody or fragment is for increasing plasma haemoglobin in a human or animal subject.

In an example, the antibody or fragment is for increasing mean corpuscular haemoglobin (MCH) in a human or animal subject.

In an example, the antibody or fragment is for increasing corpuscular volume (MCV) (and also optionally mean corpuscular haemoglobin (MCH)) in a human or animal subject.

In an example, the antibody or fragment is for increasing iron availability in a human or animal subject.

In an example, the antibody or fragment is for increasing transferrin saturation in a human or animal subject.

In an example, the antibody or fragment is for increasing transferrin binding to iron in a human or animal subject.

In an example, the antibody or fragment is for reducing the total dose of ESA (eg, EPO, Darbepoetin alfa) administered over a 4 week period to a human or animal subject for treating or preventing anaemia, osteoporosis or any other disease or condition disclosed herein.

In an example, the antibody or fragment is for reducing to ½ to ⅓ the total dose required in a control subject receiving identical treatment over the 4 week period except for administration of ESA without administration of an anti-BMP6 antagonist (eg, antibody or fragment) to a human or animal subject for treating or preventing anaemia, osteoporosis or any other disease or condition disclosed herein.

In an example, the antibody or fragment is for sparing by ½ to ⅓ the administration of ESA administered to a human or animal subject over a treatment period, eg, a 4 week period, for treating or preventing anaemia, osteoporosis or any other disease or condition disclosed herein.

Optionally, the subject suffers from anaemia of chronic inflammation or CKD. Optionally, the anaemia is anaemia of chronic inflammation in the subject.

Optionally, the antibody is an IgG4 antibody.

Optionally, the dose of ESA administered to the subject is not effective when administered in the absence of an anti-BMP6 antagonist.

Optionally the dose of ESA administered to the subject is not effective when administered in the absence of an anti-BMP6 antagonist to produce in the subject one, more or all effects selected from an
(a) Increase in haemoglobin;
(b) Increase in the mean corpuscular volume (MCV);
(c) Increase in mean corpuscular haemoglobin (MCH);
(d) Increase of iron availability; and/or
(e) Increase of transferrin saturation with iron;
   wherein the ESA and an anti-BMP6 antagonist are administered to the subject for producing said selected effect(s).

In an example, the antibody or fragment of the invention is for administration to a human or animal subject for producing in the subject one, more or all effects selected from an
(a) Increase in haemoglobin;
(b) Increase in the mean corpuscular volume (MCV);
(c) Increase in mean corpuscular haemoglobin (MCH);
(d) Increase of iron availability; and/or
(e) Increase of transferrin saturation with iron;
   wherein the ESA and an anti-BMP6 antagonist are administered to the subject for producing said selected effect(s).

In an example, the subject herein is refractory to a dose of ESA, but is responsive for treatment of anaemia, osteoporosis or another disease or condition when administered the antibody or fragment of the invention and the ESA dose.

Thus, in an example, the antibody or fragment of the invention is for administration in combination with a dose of an ESA to a human or animal subject for treating a BMP6-related disease or condition in the subject, wherein the subject is treated with the combination, but is not treatable for the disease or condition by administration of the dose of ESA in the absence of administration of said antibody or fragment.

The disease or condition may be anaemia or any other disease or condition disclosed herein.

### EXAMPLES

### Example 1

### Generation of a HepG2 Luciferase Reporter Cell Line for Studying Impact on Hamp Expression

The aim was to generate a human hepatic cell line that expresses a reporter gene under control of the hamp regulatory elements to allow testing for BMP6 induced expression of the hepcidin-encoding (hamp) gene. The complete regulatory region of about 3 kb with response elements to pSMAD (BMP) and pSTAT (IL6) has been characterised in the literature (Casanovas et al., 2014). The genomic DNA from HepG2 Cells was isolated and PCR was performed to amplify the hamp regulatory region and add restriction sites Spe1 and Xho1 (Hep Prom SPE1 Forward=AAAAAAACTAGTAAATGGCCCCATGTGGCCCCCGCCTTGTCTGC SEQ ID NO: 6); Hep Prom XHO1 Reverse=TTTTTTCTCGAGCTGTCTGGCTGTCCCACTGCTGGGTCTTGAGCTT SEQ ID NO: 7). PCR products were cloned by standard molecular biology methods into pMCS-Red Firefly Luc (ThermoFisher) plasmid vector.

The hamp regulatory region followed by the Red Firefly luc insert was re-amplified by PCR and Kpn1 and AsiS1 restriction sites introduced to allow subcloning into a PiggyBac expression vector containing a puromycin selection cassette constructed in house (Yusa et al., 2011).

HepG2 cells (ATCC) were transfected with the constructed Red Firefly Luc plasmid containing the cloned hamp regulatory region using Freestyle max transfection agents (ThermoScientific).

### Confirmation of Activity of HepG2 Hamp Reporter Cell Line

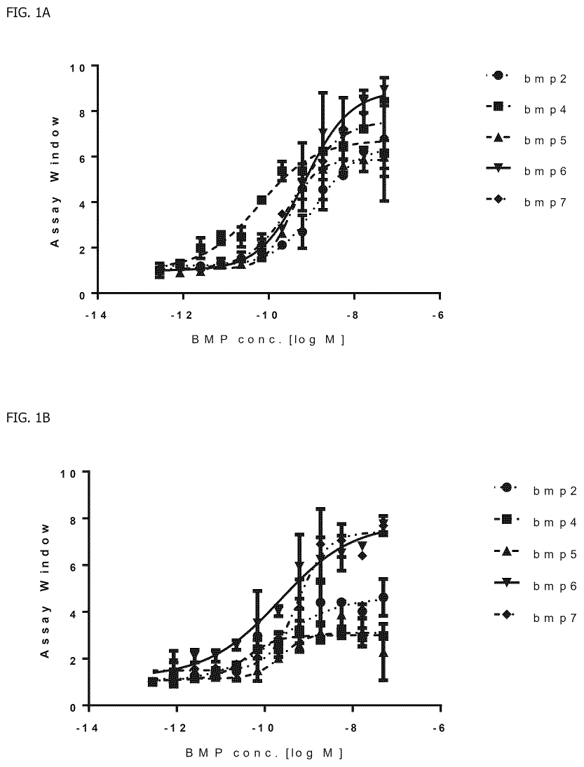

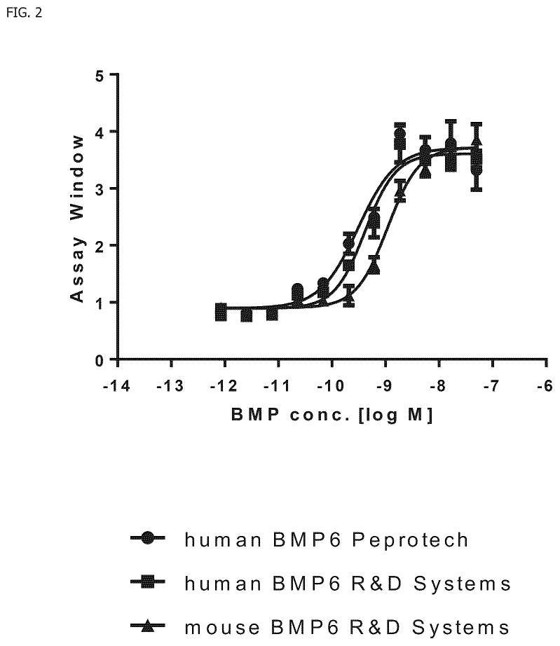

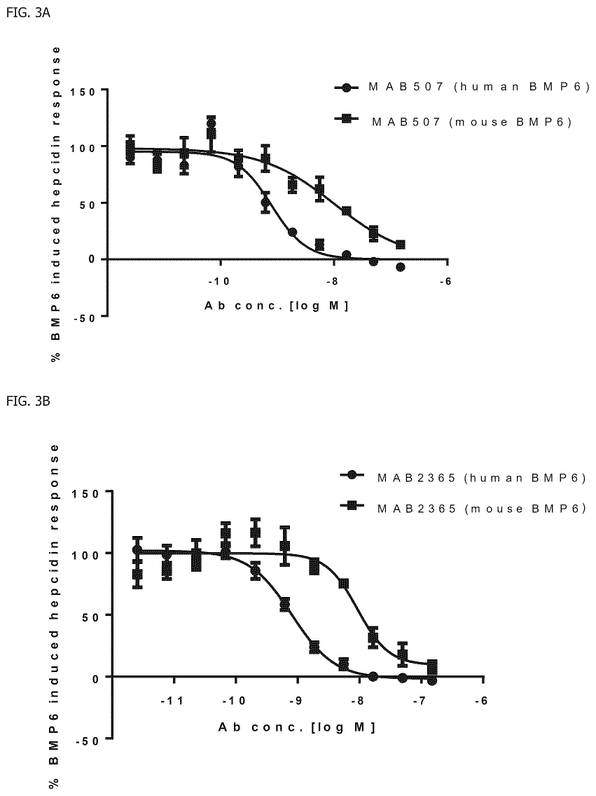

Stable cell lines carrying the Red Firefly luciferase gene under control of the entire 2.8 kB human hepcidin promoter regulatory element as described above were tested for function by stimulating the cells with various recombinant human BMP proteins known to stimulate the SMAD pathway. FIGS. 1A-1B show that human BMP2 (R&D Systems 355-BM SEQ ID NO: 493), BMP4 (R&D Systems 314-BP; SEQ ID NO: 494), BMP5 (R&D Systems 615-BMC; SEQ ID NO: 495), BMP6 (Peprotech 120-06; SEQ ID NO: 2) and BMP7 (R&D Systems 354-BP; SEQ ID NO: 496) were all able to induce the expression of the cloned luciferase reporter gene to a similar degree. BMP6 and BMP7 consistently gave the highest level of stimulation even when different media conditions were used. For some BMPs the assay window was elevated in MEM medium compared to hybridoma growth medium with 25% MEM (compare FIGS. 1A/1B)). The HepG2 luciferase reporter line was also tested with different commercially sourced batches of human and mouse BMP6 proteins (R&D Systems 507-BP; SEQ ID NO: 3, Peprotech 120-6; SEQ ID NO: 2 and R&D Systems 6325-BM; SEQ ID NO: 5) (FIG. 2). Cross-reactivity and the capacity to neutralise the activation effect of BMP6 with anti-BMP6 monoclonal antibodies MAB507 and MAB2365 (both R&D Systems) for mouse and human BMP6 respectively was also assessed using this cell line. This revealed that despite the commercially available antibodies MAB507 and MAB2365 being described by the manufacturer as human BMP6 and mouse BMP6 specific monoclonals respectively, they both were cross-reactive with BMP6 from both species albeit with slightly different potency (FIGS. 3A-3B).

### Materials and Methods for Stimulation Assay

The HepG2 hamp luciferase reporter cell line was seeded into two 96 well plates, (2×10⁴ cells/well in 50 μl of MEM media (Minimum Essential Medium— #31095-029, Gibco); 1% v/v FBS). The different BMP proteins mentioned above were diluted in 25 μl of MEM containing 1% FBS and serially diluted 1:3 starting at a concentration of 50 nM. 25 μl of MEM or 25 μl Hybridoma Media (HMM) was then added to each well depending on the experiment (equivalent to 25% of final volume). 25 μl of each dilution was then added to each well containing the cells and plates incubated for 6 h at 37 °C. After 24 hrs incubation, 100 μl of Firefly luciferase glow reagent (Thermo Scientific #16197) was added to each well. Cells were put on a shaker for three minutes and then incubated at room temperature in the dark for ten minutes. Luminescence was measured using an Envision™ reader (Perkin Elmer). In some cases (FIG. 2) the experiment was carried out in a total volume of 60 μl using 25% HMM or a fixed final concentration of 1 nM BMP6 and antibody dilutions prepared in HMM for a final concentration of 25% HMM (FIGS. 3A-3B).

### Example 2

### Immunisation and Generation of Human Anti-BMP6 Monoclonal Antibodies Using Kymouse™

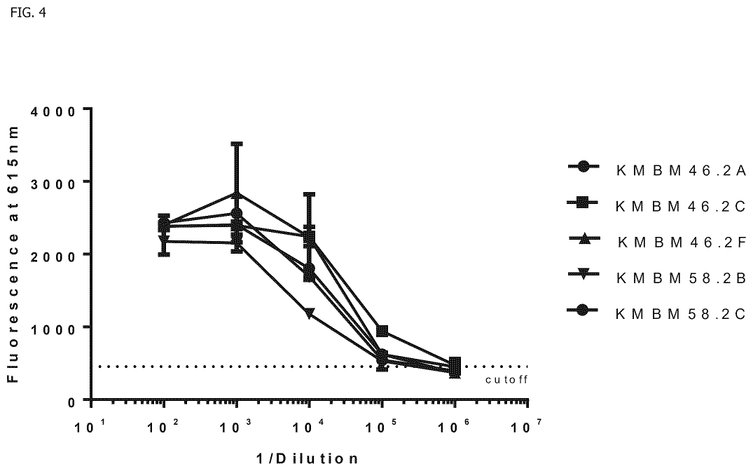

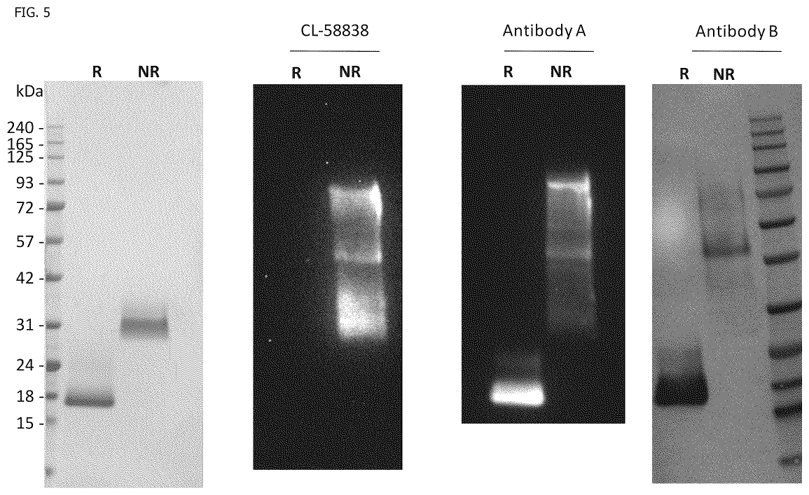

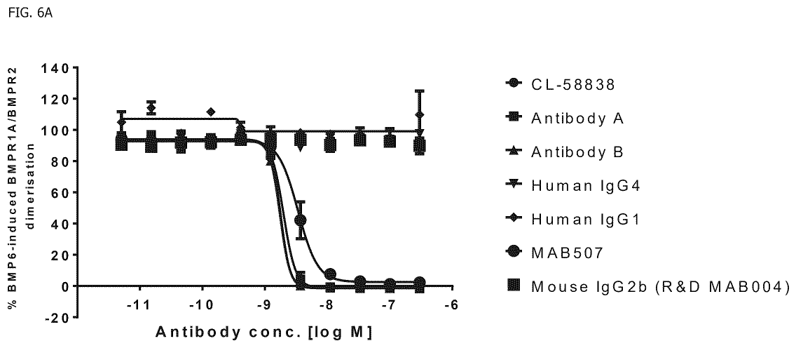

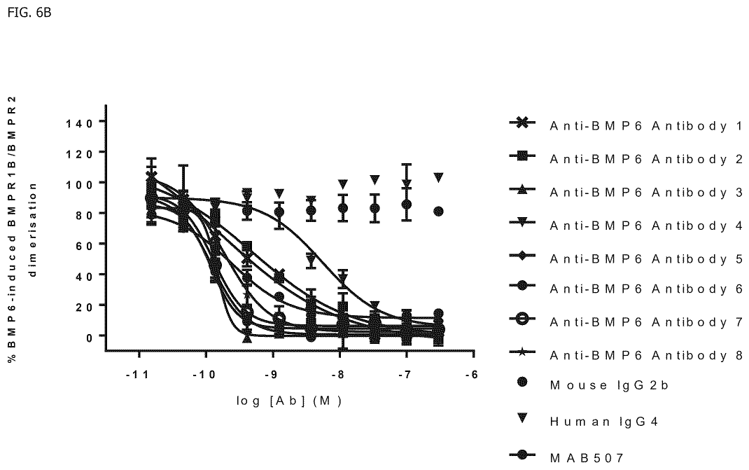

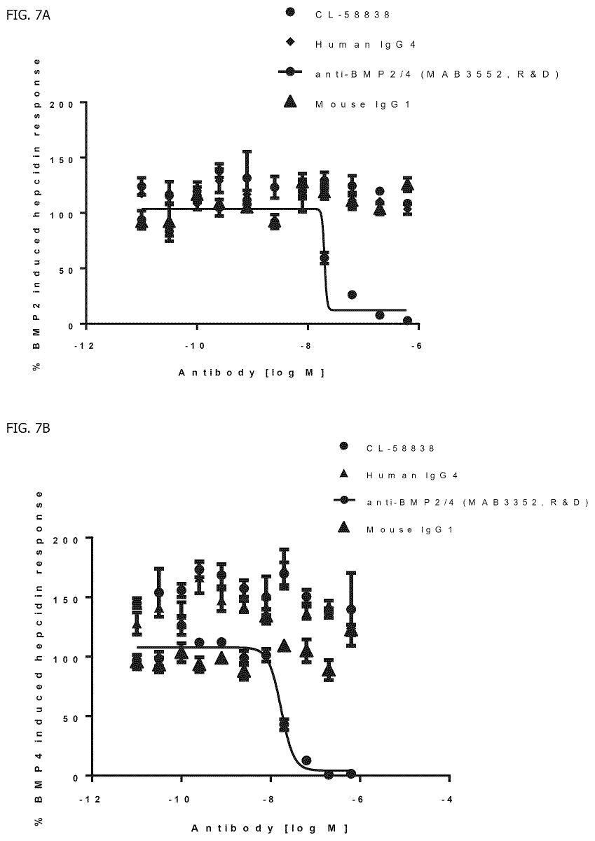

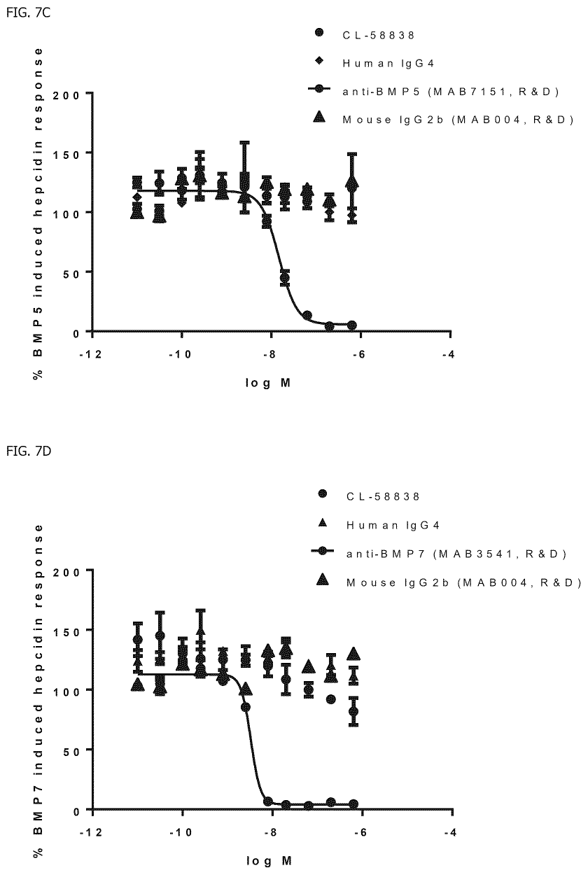

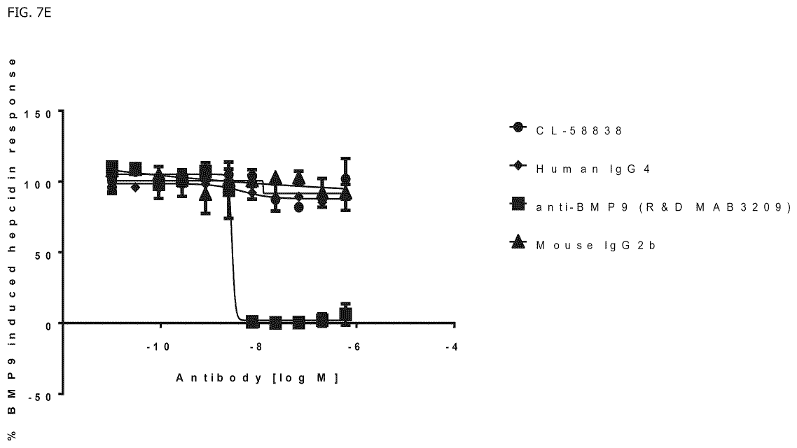

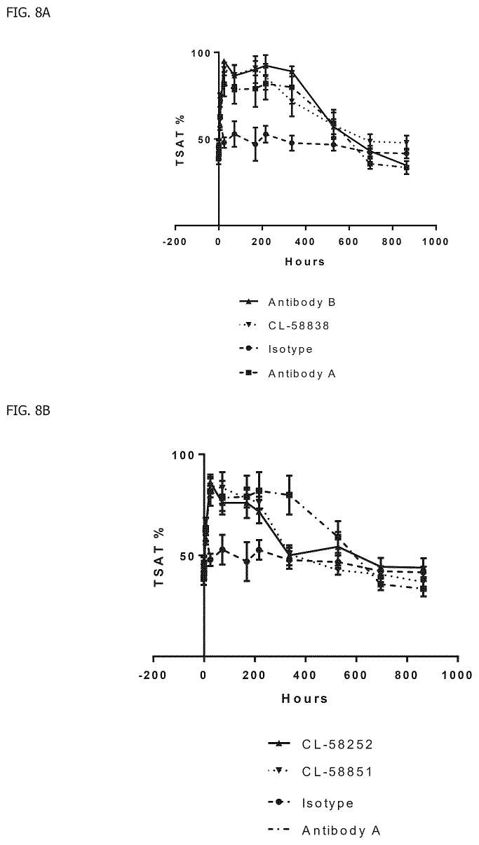

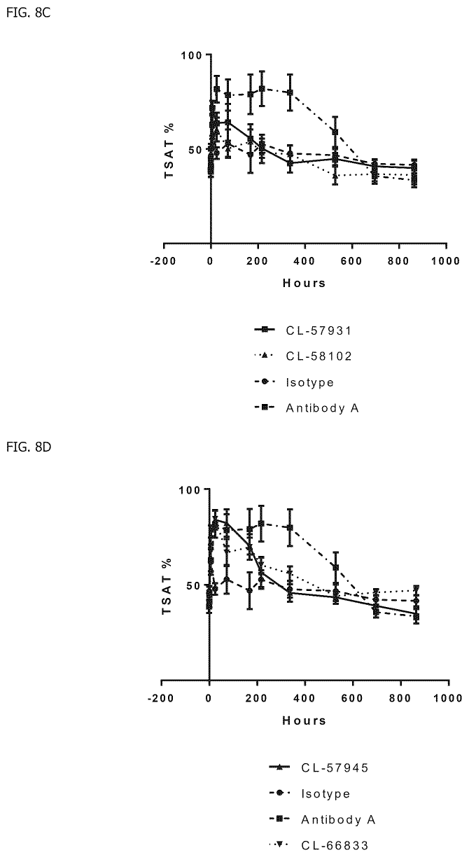

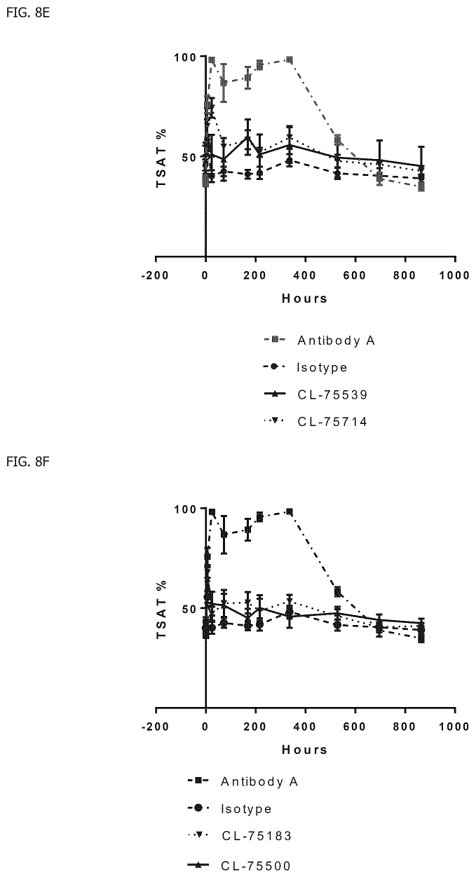

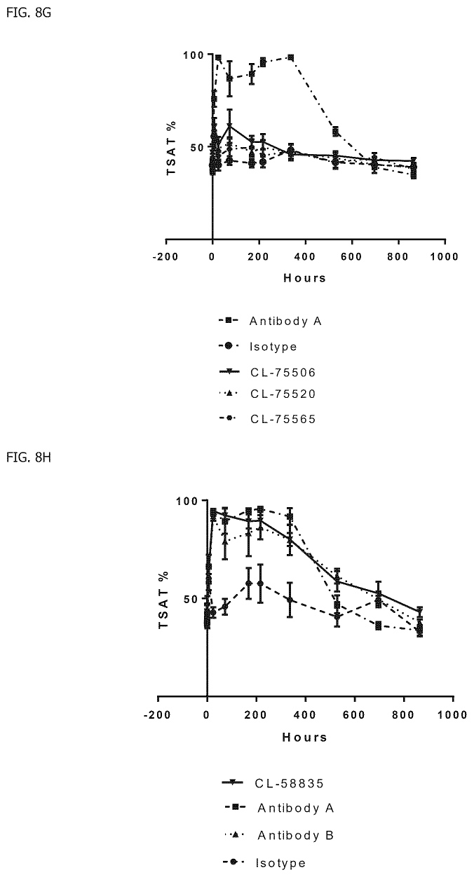

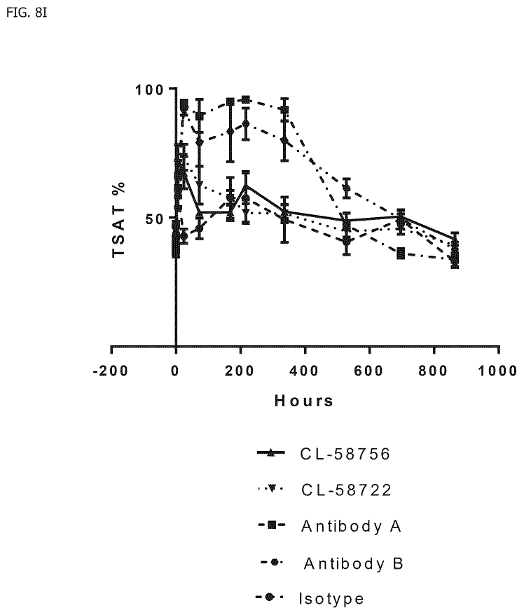

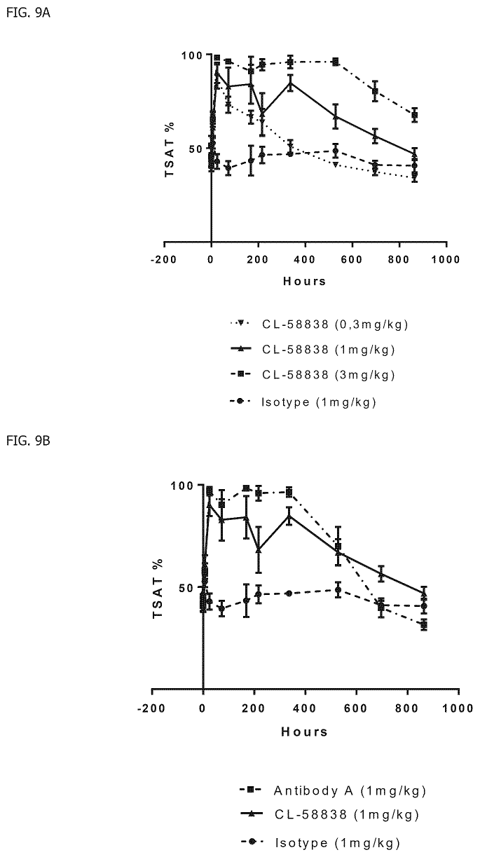

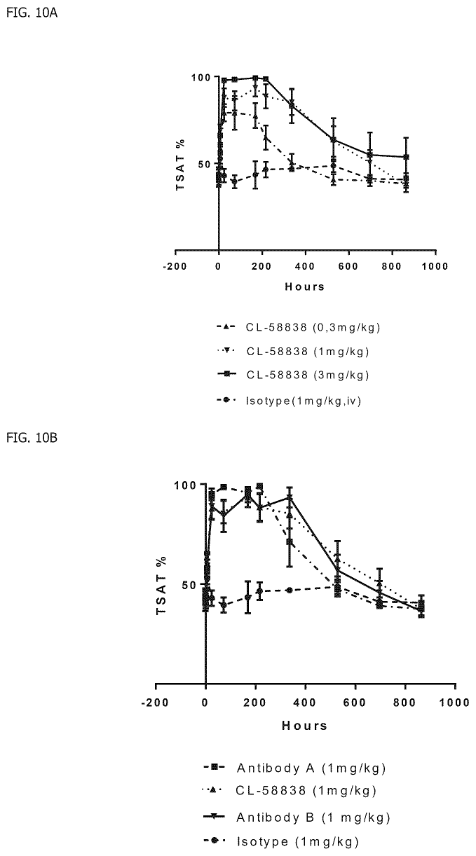

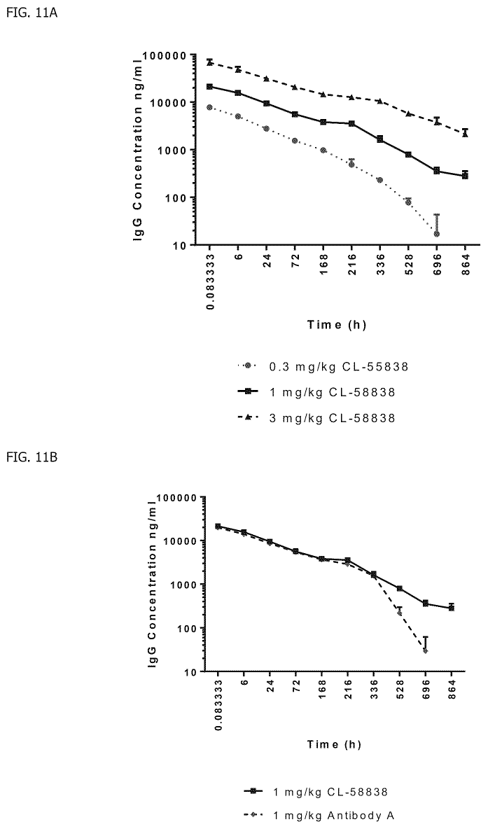

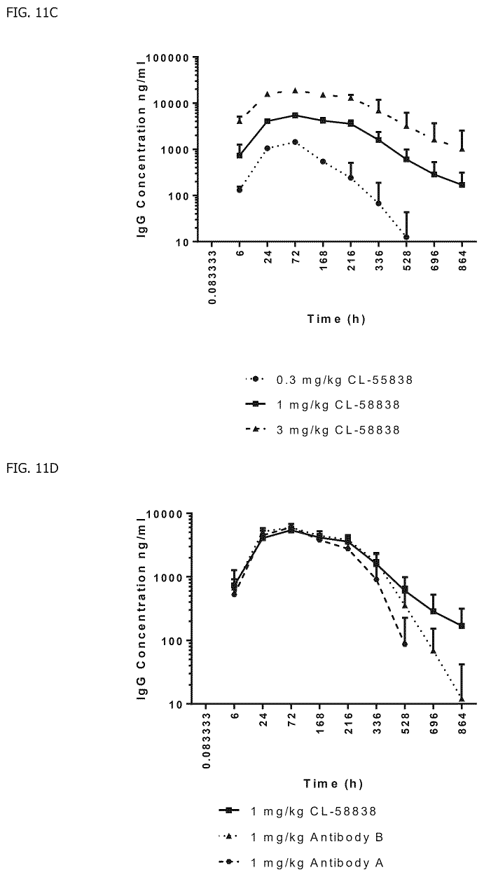

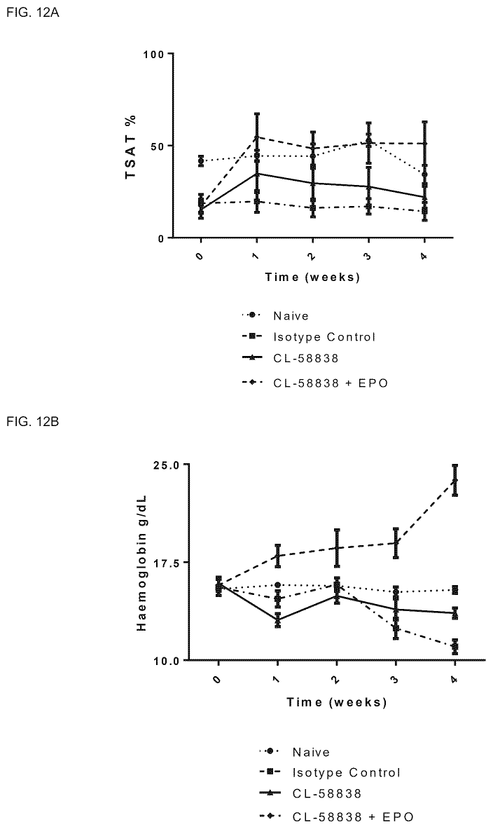

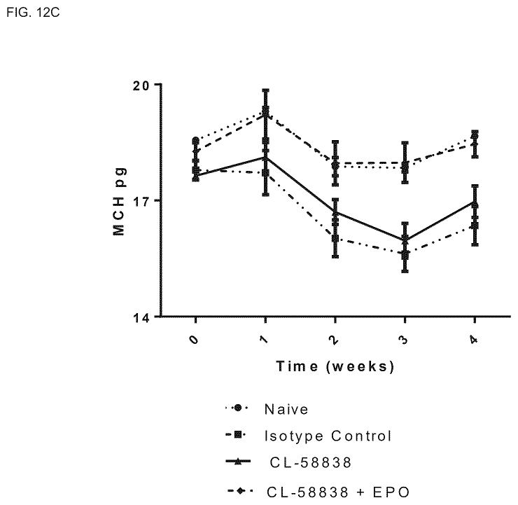

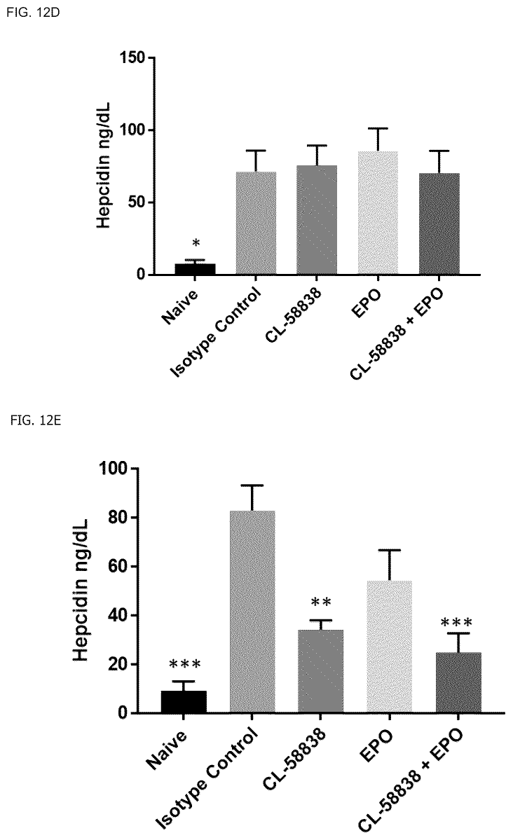

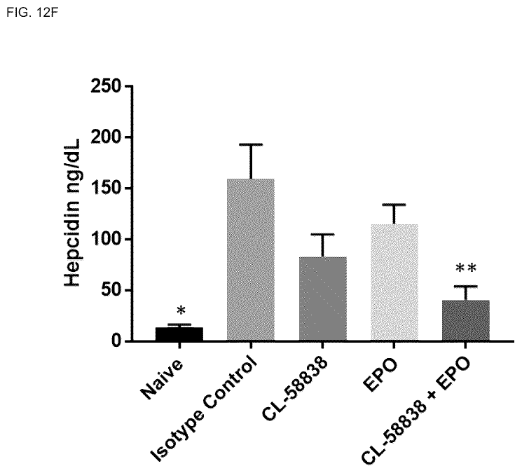

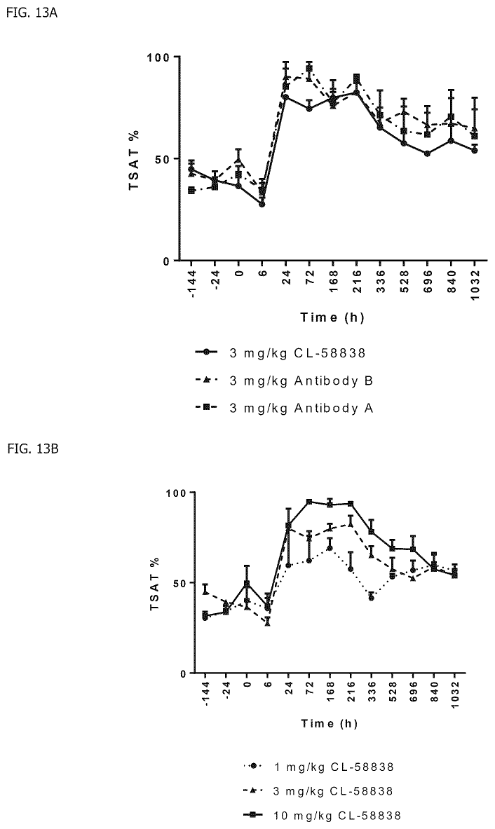

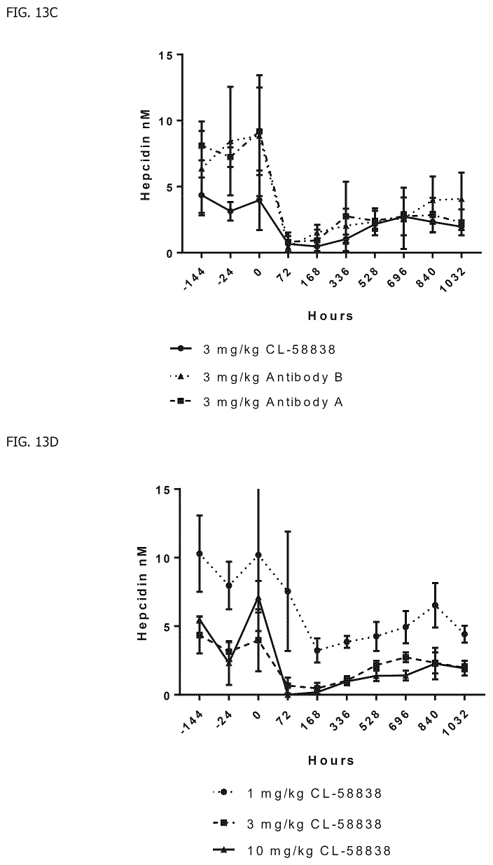

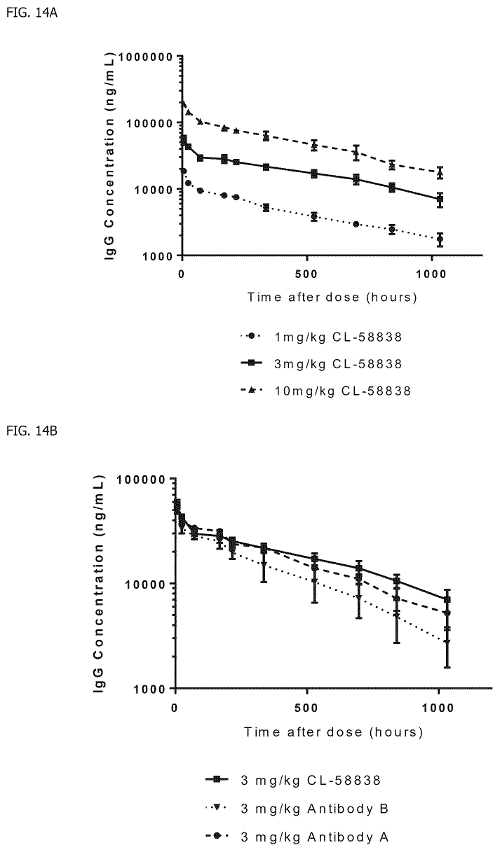

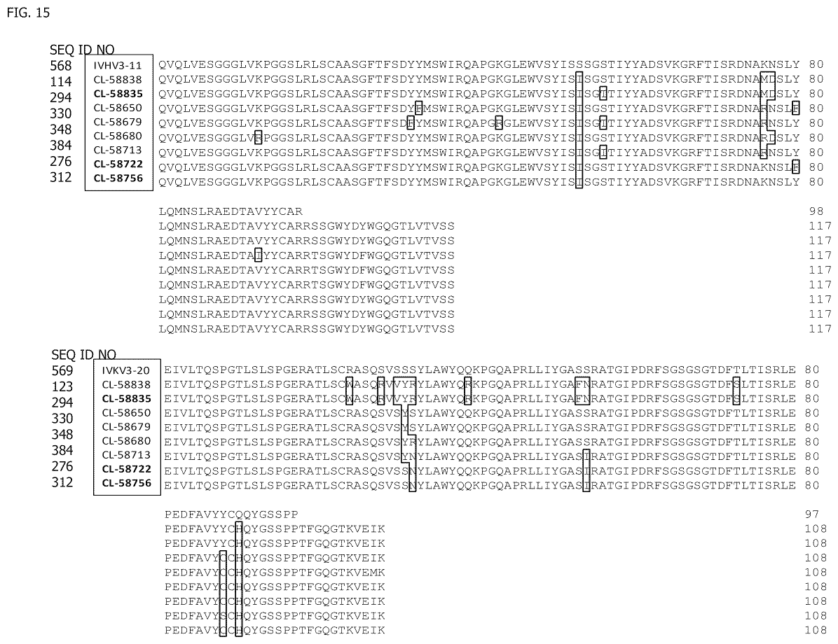

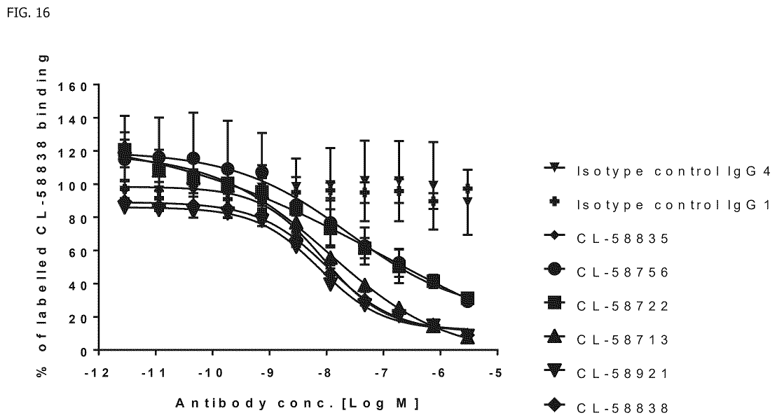

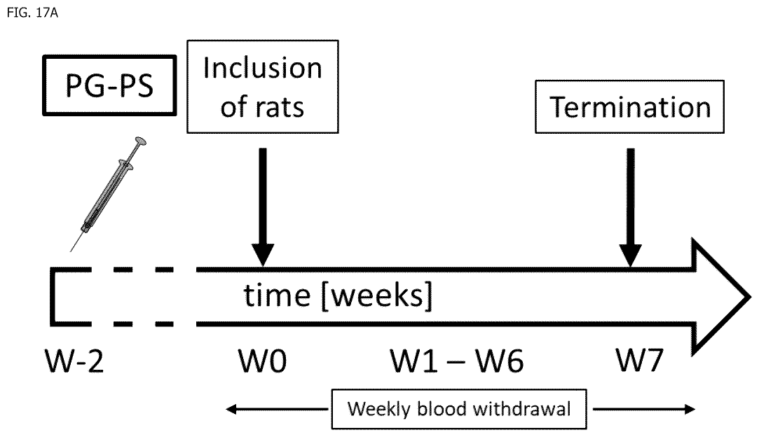

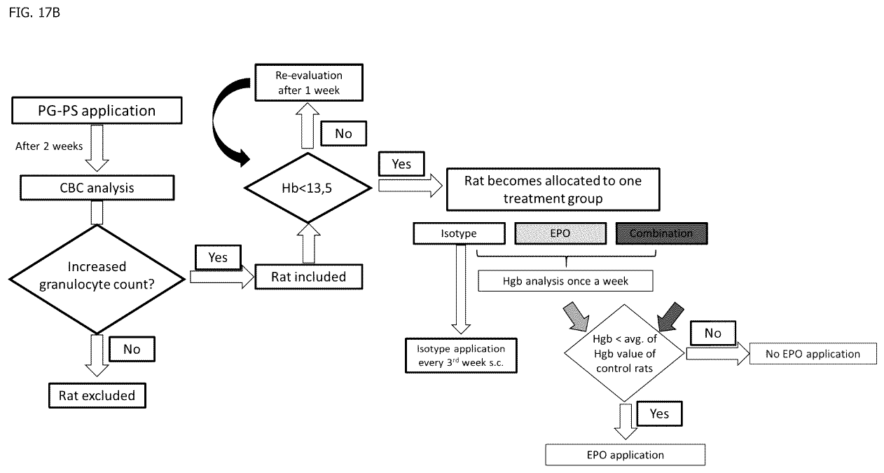

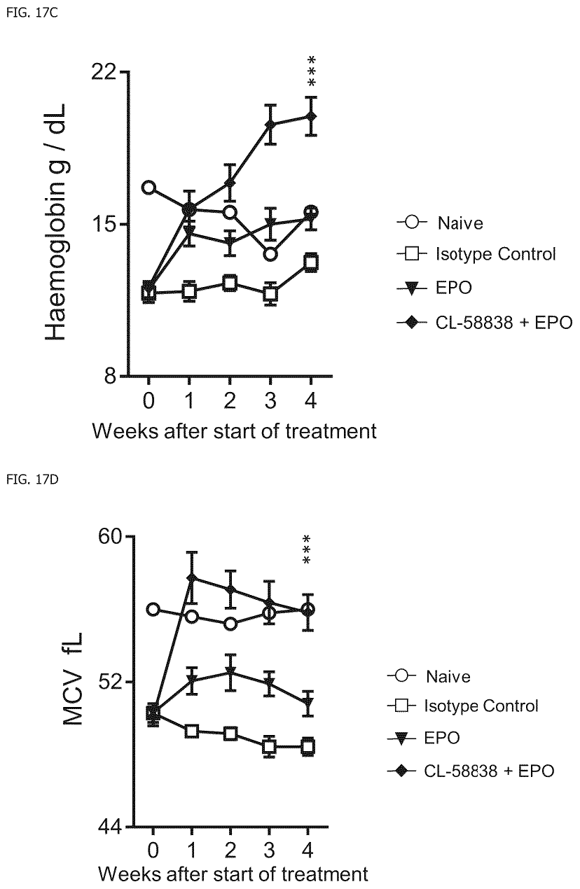

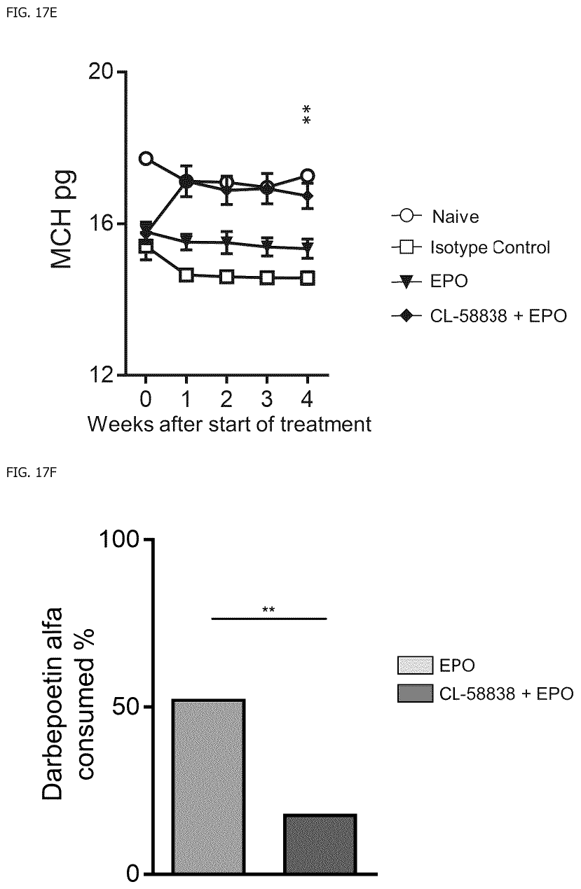

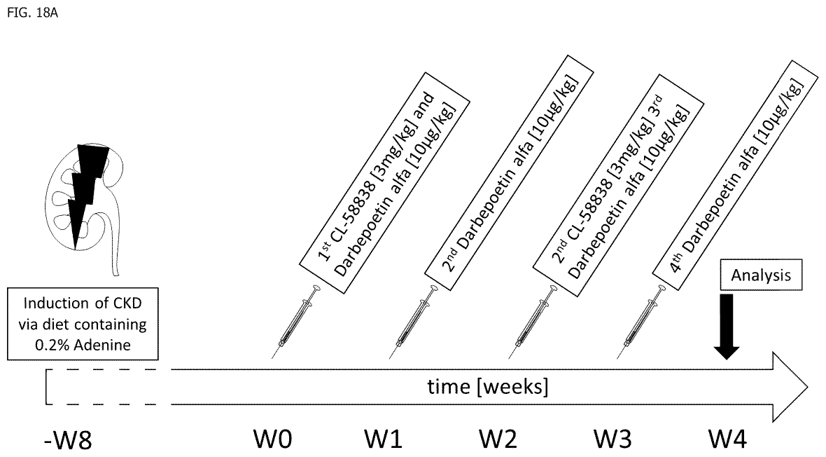

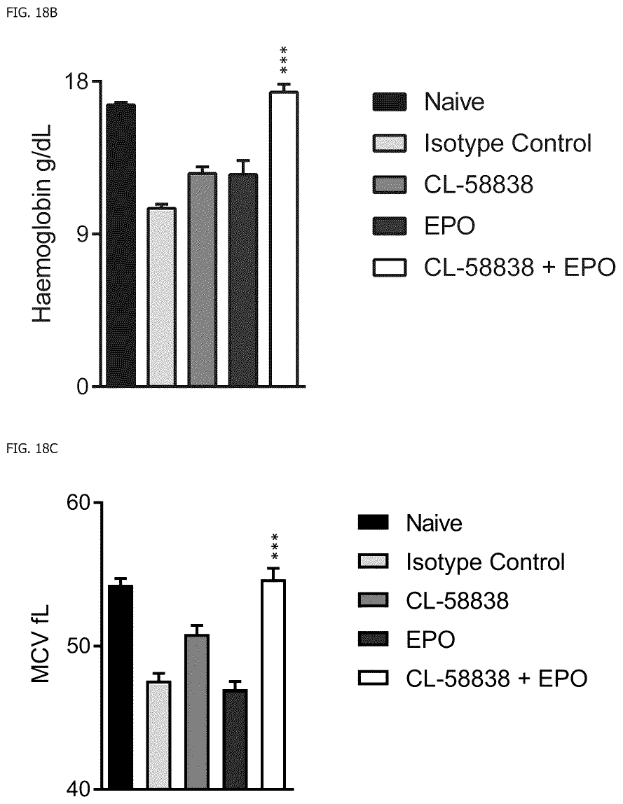

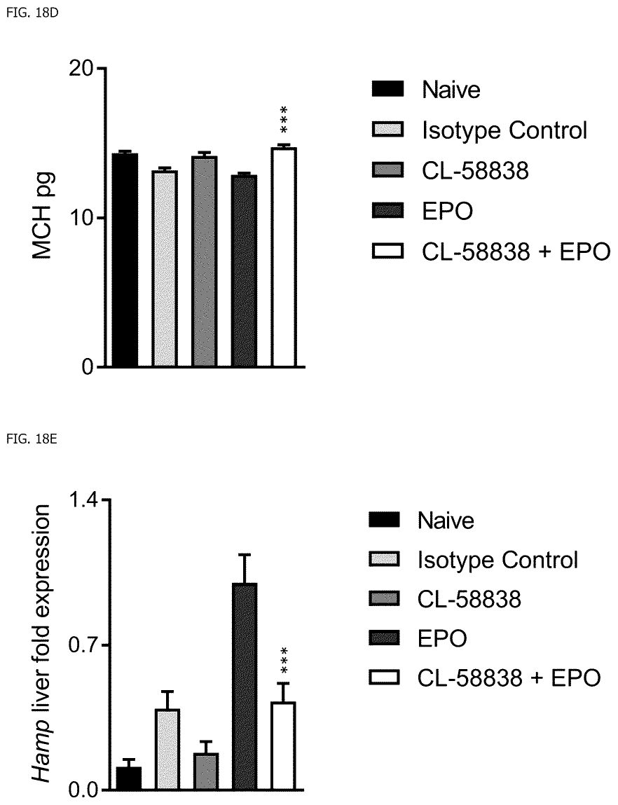

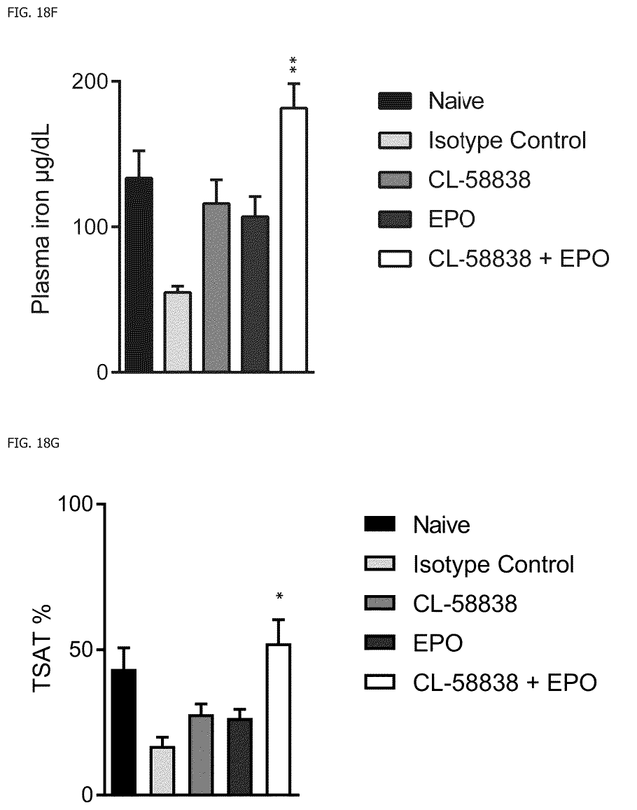

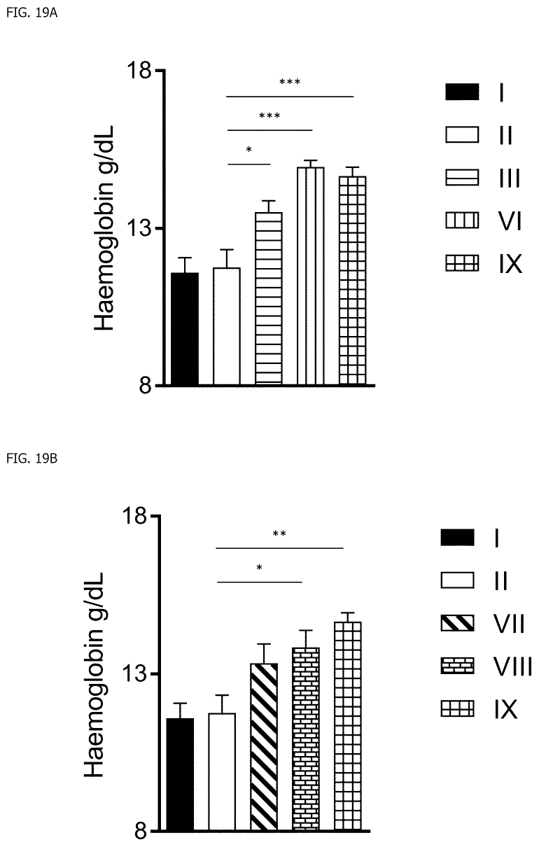

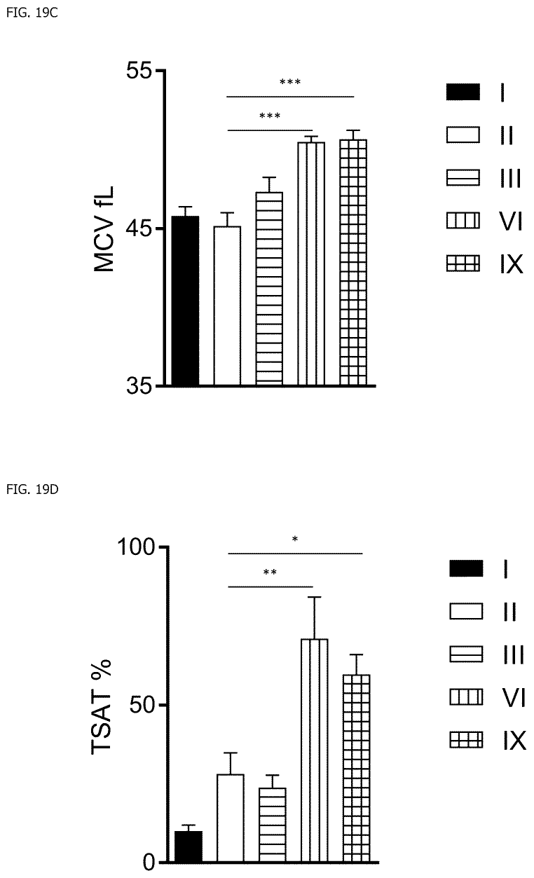

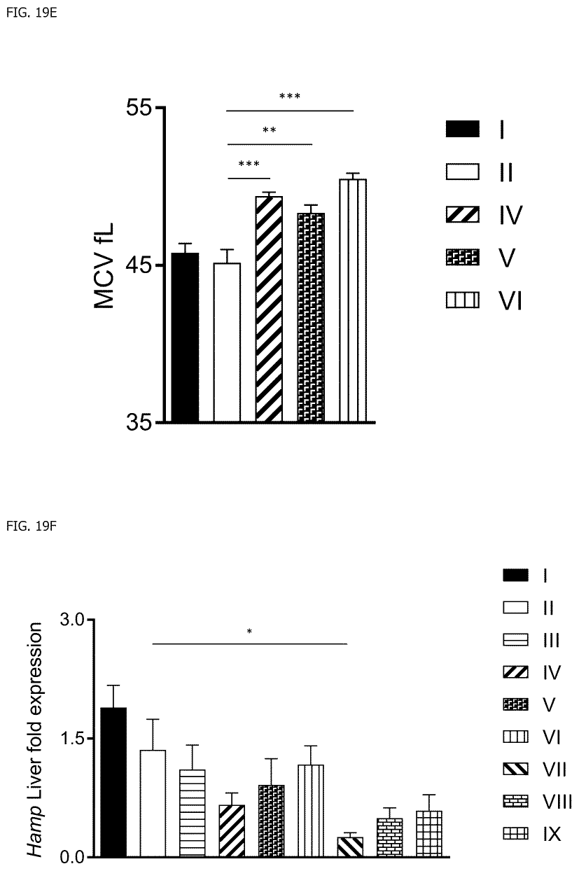

This example describes the generation of human anti-BMP6 antibodies using the Kymouse™ platform (see, e.g., WO2011/004192, WO2011/158009 and WO2013/061098). For this project, Kymouse™ HK strains containing human immunoglobulin genes producing kappa (HK) antibodies having human variable domains were generated that had the genes for murine bmp6 knocked out. These Kymouse™ HK bmp6−/− mice were immunized with recombinant human BMP6 (Peprotech 120-06; SEQ ID NO: 2) using a prime/boost regime (Table 1). At the end of each regime a final boost was applied and spleens and lymph nodes were removed about 6-7 days after. In some cases only splenocytes were used in others cells obtained from draining lymph nodes were also used (Table 2). Tissues were disaggregated into single cell suspensions for antigen-driven selection of B-cells using B-cell FACS selection technology. Where serum titres were determined during the course of the immunisation a DELFIA assay was used as outlined below. Example serum titres for anti-BMP6 IgG after 3 boosts shown for 5 animals (KM089) in FIG. 4. As evident from Table 1, KM152 was a straight repeat from KM089 and produced similar titres (data not shown).

TABLE 1
Overview Immunisation Regimes using Kymouse ™ HK bmp6 −/− mice

| Immunisation | Kymouse ™ strain | Prime/boost regime |
|---|---|---|
| KM089 | HK bmp6 −/− | Prime followed by 3-4 |
| | | boosts; |
| KM152 | HK bmp6 −/− | Prime followed by 2-3 |
| | | boosts; |

### Determination of Serum Titres by DELFIA®:

Serum titres for anti-BMP6 antibodies were determined by a reverse DELFIA® assay (Perkin Elmer) where antibodies were captured via the Fc domain (goat anti-mouse IgG; Southern Biotech 1030-01), blocked with blocking buffer (PBS containing 1% w/v BSA) and then biotinylated BMP6 (Peprotech 120-06 SEQ ID NO: 2)) added to the wells. Bound BMP6 was detected using DELFIA Eu—N1 Streptavidin (Perkin Elmer) at 1:1000 dilution. Enhancement solution was added for 5 minutes and left at room temperature in the dark and then read at 615 nm (Perkin Elmer Envision). Plates were washed between each incubation step 3× with wash buffer (PBS 0.1% v/v Tween). Anti-human BMP6 antibody (R&D systems MAB507) was used as a positive control.

### Murine Tissue Isolation and Preparation:

Selected animals based on anti-BMP6 titres were given a final boost and spleens excised 6-8 days later, washed in 1×PBS and kept on ice until further processing. Tissues were prepared in buffer containing 1×PBS (Invitrogen) and 3% heat-inactivated FBS (Invitrogen). Splenocytes were dispersed by mashing the tissue through a 40 μm strainer (BD Falcon) and rinsing with 30 ml 3% FBS/PBS buffer before centrifugation at 500 g for 10 minutes at 4 °C. To remove red blood cells, the pelleted splenocytes were resuspended in 1 ml of ACK Lysis Buffer (Invitrogen). After 2 minutes of incubation at room temperature, the lysis reaction was stopped by addition of 9 ml of 3% FBS/1λPBS buffer. Cell clumps were filtered out with a 40 μm strainer. The remaining splenocytes were pelleted for further procedures.

### BCT Sorting and Processing for Expression

For KM089 one B-cell sort was carried out using 4 animals and for KM152 two sorts were carried out with a total of 6 animals (Table 2). Splenocytes, and in some cases also lymph nodes, were prepared from selected animals and subjected to antigen specific B-cell selection and sorting. For this, biotinylated BMP6 material was generated from recombinantly produced human BMP6 (Peprotech 120-06 SEQ ID NO: 2) and the material tested for binding activity with anti-BMP6 MAB507 (R&D Systems; data not shown). Background binding of the labelled material was assessed by measuring the binding of the labelled BMP6 material to B-cells isolated from animals immunised with an unrelated immunogen. Antigen positive and B-cell marker positive single B-cells were sorted into 96 well plates and immediately frozen for molecular biology processing. B-cell technology (see WO2015040401 for a general description) was used to amplify V-regions from those antigen-selected B-cells. From this primary PCR product V-regions were recovered by further PCR and standard molecular biology methods used in the art and cloned into mammalian expression vectors with the recovered heavy and light chain pairing as recombinant chimeric IgG or as chimeric Fab fragments in HEK cells in 96 well culture plates. After 6-8 days of culture supernatants were tested for binding or neutralisation activity as outlined below. In some cases the primary PCR product derived from antigen selected single B-cells was also subjected to NGS sequencing and V-regions generated from synthetic DNA, cloned into mammalian expression vectors and plasmid DNA prepared for transfection of mammalian cells for expression.

### Example 3

### Primary Screening

Primary screening was carried out by homogeneous time resolved FRET (HTRF) to establish the binding of the recovered antibody to human BMP6 (Peprotech 120-06 SEQ ID NO: 2) and in some cases also by surface plasmon resonance (SPR) using human Fab fragments binding to immobilised human BMP6. In some instances, the HepG2-based hamp luciferase reporter gene assay described in Example 1 using human BMP6 (Peprotech 120-06 SEQ ID NO: 2) was used in a primary screen setting to directly select for neutralising antibodies (Table 2). Where off-rate ranking was used as a selection criterion a threshold for the off-rate (kd) of a least 10-4 [1/s] at 37 °C was used.

TABLE 2
Summary of primary screens and outcomes carried out on culture supernatants or
purified IgG or Fab fragments recovered and expressed in mammalian cells

| | Input into Ag- | | | | |
|---|---|---|---|---|---|
| Immunisation | driven B-cell | | | Hit selection | |
| Regime | sorting | Input material | Primary screen | criteria | Primary hits |
| KM089-B1 | spleens from 4 | 1149 supernatants | HTRF binding to | >10% effect over | 508 |
| | mice | produced from | human BMP6 | background | |
| | | single B-cells | | | |
| | | cloning/expression | | | |
| | | at 3 different | | | |
| | | dilutions | | | |
| KM152-B1 | spleens from 4 | 117 purified | Binding to | >10% binding | 23 SPR hits |
| | mice | clones recovered | human BMP6 | over background | selected |
| | | from single B-cell | (HTRF) and off- | (HTRF; 69 hits) | (including 14 |
| | | PCR expressed as | rate (SPR) | and off-rate | also positive |
| | | IgG and Fab | | <1 × 10⁻⁴ (SPR; 23 | for HTRF) |
| | | fragments | | hits) | |
| KM152-B2 | spleens and | 76 purified clones | Binding to | Off-rate <1 × 10⁻⁴ | 12 unique |
| | lymph nodes | recovered from | human BMP6 | (SPR; 12 hits) | clones |
| | from 2 mice | single B-cell PCR | off-rate (SPR) | and | selected |
| | | expressed as IgG | and BMP6 | neutralisation of | based on |
| | | and Fab fragments | neutralisation | BMP6 induced | sequence |
| | | | (HepG2 hamp | luciferase | analysis |
| | | | luciferase | reporter gene | (including 5 |
| | | | reporter assay | expression (23 | clones |
| | | | 11-point | hits) | positive in |
| | | | titration) | | both |
| | | | | | screens) |

### HTRF Assay for Binding to Human BMP6

Biotinylated BMP6 (Peprotech 120-06 SEQ ID NO: 2)) was detected with streptavidin D2 (Cisbio (Cat No 610SADLB) and antibodies bound to BMP6 were detected using anti-mouse IgG (Southern Biotech #1030-01) labelled with cryptate. MAB507 (R&D Systems) was used as a positive control and mouse IgG isotype as a negative control. Plates were read on an Envision plate reader (Perkin Elmer) and data was analysed using IDBS software. Positives were generally defined as >10% signal of positive control.

### Screening for Functional Inhibition of Human BMP6 Induced Luciferase Expression in HepG2 Cells Under Control of the Hamp Regulatory Region

In some cases the HepG2 hamp luciferase reporter gene assay described in Example 1 was used for the primary assessment of recovered IgG to assess the neutralisation capacity for BMP6. 15 ul of a known inhibitor of BMP6 signalling was used as the positive control (R&D systems MAB507; (Andriopoulos et al., 2009)) and human or mouse IgG used as the negative control. Purified antibodies were prepared in HMM 25% E Media from 150 nM, 1:3 dilution curve, 11 points) or IgG-containing supernatant samples were added to 15 ul human BMP6 (Peprotech 120-06 SEQ ID NO: 2) at 10 nM final in MEM 1% FBS and incubated 30 min at RT and then 30 ul HepG2 reporter cells added at 10 000 cells/well and incubated at 37 °C overnight. Next day 30 ul of luciferase substrate buffer (Pierce Firefly Luc One-Step Glow Assay Kit Cat Number: 16197, Perbio) was added to the whole plate and read using an Envision plate reader (Perkin Elmer).

### SPR Off-Rate Ranking Analysis

Off-rate screening was performed on a ProteOn™ XPR36 system (BioRad). Biotinylated BMP-6 (Peprotech 120-06 SEQ ID NO: 2) was captured on the NLC sensor chip surface and 50 μl of purified Fab material used as analyte diluted in 200 μl HBS-EP buffer. Off-rate analysis carried out using the software inherent to the ProteOn and all assay runs were carried out at 37°. The inventors set as a decision criterion, that antibodies that displayed of-rates of <1×10-4 [1/s] or better were identified as a positive.

### Example 4

### Secondary Screening and Selection of Lead Panels

Table 3 summarises the secondary screening and further selection criteria devised by the inventors and applied that led to the selection of lead panels for in vivo analysis. This re-testing involved the use of the HepG2 hamp luciferase reporter gene assay using a BMP6 stimulus and/or SPR off-rate ranking where not applied already in the primary screen. In addition, all selected hits were tested for specificity for BMP6 by testing their capacity to neutralise related BMP molecules in the HepG2 reporter gene assay. In the first instance BMP5 (SEQ ID NO: 495) and BMP7 (SEQ ID NO: 496) were tested due to their higher amino acid homology to BMP6 (human BMP5 shares 81% and human BMP7 72% amino acid homology with human BMP6 in the mature protein). As outlined in Example 1, all of these BMPs are able to effectively trigger the luciferase reporter gene expression in HepG2 cells under control of the hamp regulatory element region. MAB507 was used in this assay as a control since this mAb has previously shown cross-reactivity with BMP5 and BMP7 in particular (Andriopoulos et al., 2009). The inventors set as a decision criterion, that antibodies that showed consistent neutralisation of either BMP5 (SEQ ID NO: 495) and BMP7 (SEQ ID NO: 496) or both were excluded. In the case of KM089-B1 secondary screen the HepG2 reporter gene assay was also performed with murine BMP6 (R&D Systems; SEQ ID NO: 5) to test for murine cross reactivity, however, for subsequent campaigns this screen was dropped from the secondary screens since murine cross reactivity was commonly observed across all hits presumably due to the high homology between human and mouse BMP6.

TABLE 3
Summary of secondary and further assessments of primary hits carried out on culture
supernatants or purified IgG or Fab fragments recovered and expressed in mammalian cells

| | Secondary | | | | |
|---|---|---|---|---|---|
| Clone pools | screen input | Secondary screen I | Outcome | Secondary screen II | Outcome |
| KM089-B1 | 508 HTRF hit | Human and mouse | 237 >50% | 237 purified Fabs | 23 combined hits; |
| PCR recovery | supernatants | BMP6 | neutralisation | off-rate screened | sequence |
| | containing IgG | neutralisation | and murine | (SPR) with off-rate | analysis, |
| | | (HepG2 hamp | cross-reactivity | <1 × 10⁻⁴ and BMP6 | expression data |
| | | luciferase reporter | | neutralisation | and testing for |
| | | assay (single | | (HepG2 hamp | specificity for |
| | | point)) | | luciferase reporter | BMP6 resulted in |
| | | | | assay (11-point)) | 6 antibodies |
| | | | | | selected for in |
| | | | | | vivo analysis |
| | | | | | (Table 7) |
| KM152-B1 | 157 new | Off-rate screen | 60 hits (16 off- | 60 secondary hits | Sequence analysis |
| NGS and PCR | purified | (SPR) and BMP6 | rate SPR and | from NGS were | resulted in 4 |
| recovery | antibodies | neutralisation | 44 HepG2 | combined with 23 | unique combined |
| | identified by | (HepG2 hamp | hamp | hits recovered from | hits; 1 hit |
| | next- | luciferase reporter | luciferase | single B-cell PCR | selected for in |
| | generation | assay (11-point)) | reporter assay) | primary screen | vivo assessment |
| | sequencing | | | (Table 2) | following |
| | (NGS) and | | | | expression and |
| | network | | | | specificity testing |
| | analysis | | | | for BMP6 (Table |
| | | | | | 7) |
| KM152-B2 | 12 selected | BMP neutralisation | 7 selected for | — | — |
| PCR recovery | hits (Table 2) | (HepG2 hamp | in vivo | | |
| | | luciferase reporter | assessment | | |
| | | assay (11-point)) | (Table 8) | | |
| | | using BMP5, BMP6 | | | |
| | | and BMP7 | | | |
| All | 21 purified | BMP neutralisation | 10 antibodies | 4 antibodies | 3 antibodies |
| campaigns | antibodies | (HepG2 hamp | identified as | selected for | selected for in |
| NGS and | identified with | luciferase reporter | neutralisers of | expression after | vivo assessment |
| network | highly related | assay (11-point)) | BMP6; 3 | assessing potency | following |
| analysis | amino acid | using BMP5, BMP6 | excluded due | for BMP6 | sequence |
| | sequences to | and BMP7 | to cross- | neutralisation | comparison with |
| | CL-58838 | | reactivity with | (HepG2 hamp | previously tested |
| | | | BMP7 (Table 6) | luciferase reporter | clones and |
| | | | | assay (11-point)) in | expression (Table |
| | | | | comparison with CL- | 9) |
| | | | | 58838 | |

### Selection of Lead Panels for In Vivo Testing

23 combined hits were identified from KM089 secondary screening (Table 3). 4 clones were also identified from KM152-B1 with a combination of secondary screening the primary hits from single B-cell PCR and an NGS analysis of all sorted antigen specific B-cells (Table 3). These 27 antibodies were subjected to a detailed sequence analysis to identify unique clonal sequences and sequences with no obvious developability liabilities in the judgement of the inventors. Following these gating criteria, 12 antibodies were chosen by the inventors, re-expressed and purified as fully human IgG4 (SEQ ID NO: 454). Two of the 12 antibodies showed signs of expression or post-purification quality issues and were therefore not pursued further. The remaining 10 antibodies were then tested for specificity for human BMP6 and neutralisation potency of human BMP6 using the HepG2 hamp luciferase reporter assay (Table 4).

TABLE 4
Lead selection from 10 antibodies derived from KM089 and KM152 and assessed in
secondary screen for selectivity for human BMP6; antibodies selected for in vivo analysis shown in
bold)

| | | hBMP5 | hBMP5 | hBMP7 | hBMP7 |
|---|---|---|---|---|---|
| Antibody | | neutralisation | neutralisation | neutralisation | neutralisation |
| ID | Origin | (n = 1) | (n = 2) | (n = 1) | (n = 2) |
| CL-66833 | KM152- | No | No | No | No |
| | B1 | | | | |
| CL-57890 | KM089 | Incomplete | Incomplete | Incomplete curve | Incomplete curve |
| | | curve | curve | Max response | Max response |
| | | Max response | Max response | 59.8% | 24.4% |
| | | 20% | 68.5% | | |
| CL-57931 | KM089 | No | No | No | No |
| CL-58838 | KM089 | No | No | Incomplete curve | No |
| | | | | Max response 53% | |
| CL-58851 | KM089 | No | No | No | No |
| CL-58252 | KM089 | No | No | No | No |
| CL-58102 | KM089 | No | No | No | No |
| CL-57859 | KM089 | Yes, EC50 | Incomplete | Incomplete curve | Incomplete curve |
| | | 4.5 nM | curve | Max response | Max response |
| | | | Max response | 13.2% | 34.1% |
| | | | 8% | | |
| CL-58832 | KM089 | Incomplete | Incomplete | Incomplete curve | Incomplete curve |
| | | curve | curve | Max response | Max response |
| | | Max response | Max response | 16.9% | 57.7% |
| | | 9% | 11.7% | | |
| CL-57945 | KM089 | No | No | Incomplete curve | No |
| | | | | Max response | |
| | | | | 39.1% | |

Following this, 7 clones with no observed cross reactivity to BMP5 (R&D Systems 615-BMC; SEQ ID NO: 495) or BMP7 (R&D Systems 354-BP; SEQ ID NO: 496) (shown in bold Table 4) were progressed to in vivo rat studies (Example 11) and therefore re-expressed in scaled-up CHO suspension cells and purified for in vivo use.

For KM152-B2 12 combined hits selected after primary neutralisation screening (Table 2) were re-expressed. This purified lead panel was further tested by comparing the potency for neutralising human BMP6 (Peprotech 120-06; SEQ ID NO: 2) in 11-diluton point curves in the HepG2 hamp luc reporter assay with the best leads from KM089 and also considering neutralisation for BMP5 (R&D Systems 615-BMC; SEW ID NO: 495) and BMP7 (R&D Systems 354-BP; SEQ ID NO: 496). From this, 5 antibodies were excluded that did not show superior potency to previous leads from KM089 leaving 7 clones for in vivo testing (data not shown; Table 5).

TABLE 5
12 antibodies selected from KM152-B2 assessed in
secondary screen for selectivity for human BMP6;
antibodies selected for in vivo analysis shown in bold)

| Antibody | | hBMP5 | hBMP7 | Selected for in |
|---|---|---|---|---|
| ID | Origin | neutralisation | neutralisation | vivo assessment |
| CL-75714 | KM152-B2 | No | No | Yes |
| CL-75605 | KM152-B2 | No | No | No; expression |
| | | | | issues; no |
| | | | | superior |
| | | | | neutralisation |
| CL-75565 | KM152-B2 | No | No | Yes |
| CL-75539 | KM152-B2 | No | No | Yes |
| CL-75520 | KM152-B2 | No | No | Yes |
| CL-75519 | KM152-B2 | No | No | No; expression |
| | | | | issues; no |
| | | | | superior |
| | | | | neutralisation |
| CL-75511 | KM152-B2 | No | No | No; expression |
| | | | | issues; no |
| | | | | superior |
| | | | | neutralisation |
| CL-75506 | KM152-B2 | No | No | Yes |
| CL-75500 | KM152-B2 | No | No | Yes |
| CL-75496 | KM152-B2 | No | No | No; no superior |
| | | | | neutralisation |
| CL-75194 | KM152-B2 | No | No | No; no superior |
| | | | | neutralisation |
| CL-75183 | KM152-B2 | No | No | Yes |

Following the analysis of the first set of 7 antibodies in vivo (Table 4) and observing superior activity for CL-58838 (see Example 11), the decision was taken to use data obtained by next generation sequencing (NGS) to potentially expand the panel of lead clones by identifying antibodies that were based on the Vh (SEQ ID NO: 114) and Vk (SEQ ID NO: 123) class sequences of this antibody. Paired Vh and Vk chains obtained from NGS on the amplified V-regions after B-cell sorting were analysed for sequences with high homology to CL-58838 with the aim of obtaining slightly mutated yet highly related antibody sequences to CL-58838. The search was driven by having identical use of human V, D and J-regions (IGHV3-11*01, IGHD6-19*01, IGHJ4*02/IGKV3-20*01, IGKJ1*01) and identical CDRH3 sequence. A total of 21 antibodies were identified by this method, expressed, purified and assessed for function, specificity for BMP6 and potency (Table 3). The 21 antibodies were derived from 5 different immunised mice from immunisation campaigns KM089 and KM152. Ten antibodies were initially confirmed as neutralisers of BMP6 but after assessing for specificity for BMP6 over BMP5 and BMP7 and comparing potencies for neutralising human BMP6 in the HepG2 luc assay in comparison with CL-58838 7 antibodies were selected shown in Table 6. A cross-sequence comparison with antibodies tested in all previous screening campaigns excluded another 3 antibodies that were assessed before leaving four new unique antibodies that were expressed and purified for in vivo analysis. Of these four, CL-58713 did not express at sufficient quantities at this scale and was therefore also excluded. The three remaining new antibodies were progressed to an in vivo study in rats (shown in bold Table 6 and shown in Example 17) and came from two different immunised bmp6−/− mice from immunisation KM089.

TABLE 6
Antibodies identified by NGS with same V-gene
usage and CDRH3 length as CL-58838 and
selected by functional screen

| | | hBMP5 | hBMP7 | Selected for in |
|---|---|---|---|---|
| Antibody | | neutral- | neutral- | vivo assessment |
| ID | Origin | isation | isation | (example 17) |
| CL-58722 | KM089 animal CP- | No | No | Yes |
| | 230 | | | |
| CL-58713 | KM089 animal CP- | No | No | No |
| | 230 | | | (not sufficient |
| | | | | material available |
| | | | | for in vivo study |
| CL-58921 | KM089 animal CP- | No | No | No |
| | 231 | | | (sequence same |
| | | | | as CL-58838) |
| CL-58676 | KM089 animal CP- | No | No | No |
| | 230 | | | (sequence same |
| | | | | as CL-58756) |
| CL-58835 | KM089 animal CP- | No | No | Yes |
| | 231 | | | |
| CL-71800 | KM089 animal CP- | No | No | No |
| | 231 | | | (sequence same |
| | | | | as CL-58838) |
| CL-58756 | KM089 animal CP- | No | No | Yes |
| | 230 | | | |

### Example 5

### Sequence Analysis of Lead Clones

Antibodies selected for in vivo use were analysed for the V-region gene usage and the degree of mutation introduced during the in vivo maturation process that occurs naturally during immunisation of the Kymouse™. Tables 7-9 summarise this information for the antibodies selected in Tables 4-6. All CDR definitions shown here are IMGT definitions.

TABLE 7
7 antibodies selected from KM089 and KM152-B1 for in vivo study

| | | | | | Mutations | | | | | Mutations | |
|---|---|---|---|---|---|---|---|---|---|---|---|
| | | | | | outside | | | | | outside | |
| | | | | | CDRH3 | | | | | CDRL3 | |
| | | | | CDRH3 | (IMGT) | | | | CDRL3 | (IMGT) | |
| Antibody | IGHV | IGHD | IGHJ | length | Nucleic | Amino | IGLV | IGLJ | length | Nucleic | Amino |
| ID | gene | gene | gene | (IMGT) | acid | acid | gene | gene | (IMGT) | acid | acid |
| CL- | IGHV1- | IGHD3- | IGHJ4* | 11 | 17 | 12 | IGKV1- | IGKJ1* | 9 | 7 | 5 |
| 66833 | 3*01 | 10*01 | 02 | | | | 5*03 | 01 | | | |
| CL- | IGHV1- | IGHD3- | IGHJ3* | 11 | 24 | 14 | IGKV3- | IGKJ1* | 9 | 15 | 11 |
| 57931 | 3*01 | 10*01 | 02 | | | | 20*01 | 01 | | | |
| CL- | IGHV3- | IGHD6- | IGHJ4* | 10 | 5 | 3 | IGKV3- | IGKJ1* | 9 | 21 | 9 |
| 58838 | 11*01 | 19*01 | 02 | | | | 20*01 | 01 | | | |
| CL- | IGHV1- | IGHD7- | IGHJ4* | 11 | 19 | 16 | IGKV3- | IGKJ1* | 9 | 17 | 11 |
| 58851 | 3*01 | 27*02 | 02 | | | | 20*01 | 01 | | | |
| CL- | IGHV1- | IGHD4- | IGHJ4* | 11 | 12 | 9 | IGKV1- | IGKJ1* | 9 | 2 | 1 |
| 58252 | 3*01 | 23*01 | 02 | | | | 5*03 | 01 | | | |
| CL- | IGHV1- | IGHD5- | IGHJ4* | 11 | 24 | 12 | IGKV3- | IGKJ1* | 9 | 26 | 16 |
| 58102 | 3*01 | 18*01 | 02 | | | | 20*01 | 01 | | | |
| CL- | IGHV1- | IGHD5- | IGHJ4* | 11 | 21 | 12 | IGKV3- | IGKJ1* | 9 | 15 | 11 |
| 57945 | 3*01 | 18*01 | 02 | | | | 20*01 | 01 | | | |

TABLE 8
KM152-B2 7 antibodies selected from KM152-B2 for in vivo study

| | | | | | Mutations | | | | | Mutations | |
|---|---|---|---|---|---|---|---|---|---|---|---|
| | | | | | outside | | | | | outside | |
| | | | | | CDRH3 | | | | | CDRL3 | |
| | | | | CDRH3 | (IMGT) | | | | CDRL3 | (IMGT) | |
| Antibody | IGHV | IGHD | IGHJ | length | Nucleic | Amino | IGLV | IGLJ | length | Nucleic | Amino |
| ID | gene | gene | gene | (IMGT) | acid | acid | gene | gene | (IMGT) | acid | acid |
| CL- | IGHV1- | IGHD3- | IGHJ4* | 11 | 17 | 11 | IGKV3- | IGKJ3* | 9 | 8 | 8 |
| 75714 | 3*01 | 10*01 | 02 | | | | 15*01 | 01 | | | |
| CL- | IGHV1- | IGHD3- | IGHJ4* | 11 | 2 | 2 | IGKV3- | IGKJ3* | 9 | 4 | 3 |
| 75565 | 3*01 | 10*01 | 02 | | | | 15*01 | 01 | | | |
| CL- | IGHV1- | IGHD3- | IGHJ4* | 11 | 11 | 9 | IGKV3- | IGKJ3* | 9 | 5 | 3 |
| 75539 | 3*01 | 10*01 | 02 | | | | 15*01 | 01 | | | |
| CL- | IGHV1- | IGHD3- | IGHJ4* | 13 | 9 | 6 | IGKV3- | IGKJ3* | 8 | 6 | 4 |
| 75520 | 3*01 | 22*01 | 02 | | | | 20*01 | 01 | | | |
| CL- | IGHV1- | IGHD3- | IGHJ4* | 11 | 6 | 6 | IGKV3- | IGKJ3* | 9 | 5 | 4 |
| 75506 | 3*01 | 10*01 | 02 | | | | 15*01 | 01 | | | |
| CL- | IGHV1- | IGHD3- | IGHJ4* | 11 | 10 | 5 | IGKV3- | IGKJ3* | 9 | 6 | 4 |
| 75500 | 3*01 | 10*01 | 02 | | | | 15*01 | 01 | | | |
| CL- | IGHV1- | IGHD3- | IGHJ5* | 11 | 11 | 8 | IGKV1- | IGKJ3* | 8 | 5 | 4 |
| 75183 | 3*01 | 16*02 | 02 | | | | 5*03 | 01 | | | |

TABLE 9
CL-58838-like antibodies identified by NGS and sequence mining

| | | | | | Mutations | | | | | Mutations | |
|---|---|---|---|---|---|---|---|---|---|---|---|
| | | | | | outside | | | | | outside | |
| | | | | | CDRH3 | | | | | CDRL3 | |
| | | | | CDRH3 | (IMGT) | | | | CDRL3 | (IMGT) | |
| Antibody | IGHV | IGHD | IGHJ | length | Nucleic | Amino | IGLV | IGLJ | length | Nucleic | Amino |
| ID | gene | gene | gene | (IMGT) | acid | acid | gene | gene | (IMGT) | acid | acid |
| CL- | IGHV3- | IGHD6- | IGHJ4* | 10 | 6 | 4 | IGKV3- | IGKJ1* | 9 | 22 | 10 |
| 58835 | 11*01 | 19*01 | 02 | | | | 20*01 | 01 | | | |
| CL- | IGHV3- | IGHD6- | IGHJ4* | 10 | 1 | 1 | IGKV3- | IGKJ1* | 9 | 3 | 3 |
| 58756 | 11*01 | 19*01 | 02 | | | | 20*01 | 01 | | | |
| CL- | IGHV3- | IGHD6- | IGHJ4* | 10 | 3 | 2 | IGKV3- | IGKJ1* | 9 | 3 | 3 |
| 58722 | 11*01 | 19*01 | 02 | | | | 20*01 | 01 | | | |

### Example 6

### Binding Kinetics for Antibody CL-58838 Measured by SPR

The kinetics for CL-58838 IgG4 (SEQ ID NO: 116 and SEQ ID NO: 125) binding to human BMP6 was determined by surface plasmon resonance (SPR) at 37 °C at pH 7.6. Like the other antibodies used in the Examples, the CL-58838 IgG4 antibody comprised the IgH constant region of SEQ ID NO: 454 (which we call “IgG4-PE”), which is effector function inactivated and hinge stabilised. Briefly, for this measurement biotinylated recombinant human BMP6 (Peprotech 120-06) (SEQ ID NO: 2) was immobilised on a streptavidin coated biosensor chip and binding examined using a single cycle kinetic method run at 5 different IgG concentrations. In this set-up the bivalent IgG4 antibody interacts with the dimeric BMP6 antigen which means that avidity will play a part in the overall binding kinetics measured due to potential multiple interactions occurring per molecule. The bivalent model used for the data analysis was intended to deconvolute this effect but is not as reliable as measuring true 1:1 interactions. In this set-up the KD values should therefore be referred to as “relative” affinity values. The relative KD value determined for CL-58838 by this method was 0.07 nM (Table 10).

TABLE 10
Measurement of CL-58838 IgG4 binding kinetics to
human BMP6 at 37° C. and pH 7.6

| | | | Relative |
|---|---|---|---|
| Sample | ka [1/Mx1/s] | kd [1/s] | KD [nM] |
| CL-58838 IgG4 | 5.22⁰⁶ | 3.69⁻⁰⁴ | 0.07 |

In a second experimental set-up Fab fragments of CL-58838 were generated by expression in CHO cells and the interaction was measured where the Fab fragments were the analyte in the flowcell binding to immobilised biotinylated human BMP6 (Peprotech 120-06) (SEQ ID NO: 2) on a Neutravidin Biosensor chip NLC (Biorad). Since this experimental set-up involves monomeric Fab fragments that avoid bivalent interactions this represented an interaction were avidity has very limited or no contributions to the binding kinetics and therefore true KD values could be determined (Table 11). CL-58838 showed a true KD for the interaction of Fab fragment with human BMP6 at 37 °C and pH 7.6 of around 140 pM.

TABLE 11
Representative result from two experiments measuring the
binding kinetics of a CL-58838-based Fab fragment to
immobilised human BMP6 at 37° C. and pH 7.6

| Sample | Ka [1/Mx1/s] | Kd [1/s] | KD [nM] |
|---|---|---|---|
| CL-58838 Fab | 3.64⁰⁶ | 4.99⁻⁰⁴ | 0.14 |

### Methods:

### SPR Analysis of IgG

Recombinant human BMP6 (Peprotech 120-06) (SEQ ID NO: 2) was biotinylated and immobilised on a SA Biosensor Chip (GE Healthcare) and a single cycle kinetic method was deployed running IgG at 5 different concentrations of CL-58838 (0.1, 0.5, 2.5, 12.5 and 62.5 nM) as the analyte. The binding sensorgrams were double referenced using an identical set of injections with buffer in place of the antibody. The data was fitted to the bivalent model inherent to the Biacore 8K (GE Healthcare) analysis software. The assay was run at 37 °C, using HBS-EP as the running buffer at pH 7.6.

### SPR Analysis of Fab Fragments

Human BMP6 (Peprotech 120-06) (SEQ ID NO: 2) was biotinylated and immobilised on a Neutravidin Biosensor chip NLC (Biorad 1765021). Fab fragments of CL-58838 were generated by expression in HEK cells and purified with protein G and size exclusion chromatography. The Fabs were used and as analyte at 64, 16, 4, 1 and 0.25 nM. The binding sensorgrams were double referenced using a buffer injection. Data was fitted to the 1:1 model inherent to the ProteOn XPR36 analysis software. The assay was run at 37 °C using HBS-EP as the running buffer at pH 7.6.

### Example 7

Testing of CL-58838 for Cross-Reactivity with Other BMPs

Antibody CL-58838 IgG4 (SEQ ID NO: 116 and SEQ ID NO: 125) was assessed in more detail in respect of its lack of cross-reactivity with other BMP family members. The screening strategy already effectively excluded any effects on BMP5 and BMP7, the two most related BMP amino acid sequences (81% and 72% respectively). As outlined in Example 4, the cross-reactivity was assessed using the HepG2 reporter gene assay developed in Example 1 with an extended panel to also include less homologous members of the BMP family like BMP2 (56%), BMP4 (58%) and BMP9 (54%). Assays were run at fixed final concentrations of all BMP of 10 nM that provided adequate stimulation of the hamp driven reporter gene expression and a 11-point dilution range of CL-58838 antibody starting at a final concentration of 600 nM. For BMP2 (R&D Systems 355-BM; SEQ ID NO: 493), BMP4 (R&D Systems 314-BP; SEQ ID NO: 494) and BMP9 (Peprotech 120-7; SEQ ID NO: 497) cross-reactivity testing was carried out once whereas for BMP5 (R&D Systems 615-BMC SEQ ID NO: 495) and BMP7 (R&D Systems 354-BP; SEQ ID NO: 496) n=2. One experiment is shown in each case in FIGS. 7A-7E. Results show that there was no detectable neutralisation of CL-58838 against any of the BMP tested here. Commercial control antibodies specific for the relevant BMP tested in each assay neutralised the reporter gene expression as expected (anti-BMP2/4 R&D Systems MAB3552; anti-BMP5 R&D Systems MAB7151, anti-BMP7 R&D Systems MAB3541, anti-BMP9 R&D Systems 3209).

### Method

Human BMPs were prepared at 40 nM (10 nM final assay concentration) in MEM 1% FBS. Control curves were prepared using anti-BMP2/4 (R&D Systems), anti-BMP5 (MAB7151, R&D Systems), anti-BMP7 (MAB3541, R&D Systems) and Human IgG4 isotype control (in house produced). Reference antibodies were titrated from 2.4 μM in PBS (600 nM final assay concentration) using a three-fold dilution series for the generation of 11-point curve. BMP standard curves were prepared from 800 nM in PBS (200 nM final assay concentration) using a three-fold dilution series for the generation of 11-point curve. Titrations for each molecule tested were generated by three-fold dilution, 11-points curves in PBS. 15 μL were then transferred from the dilution to the assay plates (LUC). 15 μL of PBS were added to total binding and non-specific binding wells, 15 μl of human BMPs at 40 nM were added to test wells and to total binding control well and 15 μL of MEM 1% FBS was transferred to non-specific binding control wells. Plates were left at room temperature while preparing HepG2 reporter cells. Cells were detached from flasks, pelleted and re-suspended in MEM supplemented with 1% FBS at 3.3×105 cells/ml. 30 μl of cells were added to the entire test plates at 10,000 cells/well.

Plates were incubated overnight at 37 °C, 5% CO2 and the next day, 30 μl of luciferase substrate (contained in Pierce Firefly Luc one step glow assay kit) was added to assay plate. Plates were incubated for 10 minutes at room temperature in the dark and read using the Envision (Perkin Elmer).

### Example 8

### Testing Anti-BMP6 Antibodies for Interference of Receptor Dimerization

It has been suggested in the literature that ALK2 and ALK3 are the major type I BMP receptors expressed in the liver of mice and humans (Mayeur et al., 2014; Xia et al., 2008) that enable BMP induced signalling in hepatocytes. We were therefore interested in how blocking the biological activity of BMP6 with anti-BMP6 antibodies affects the dimerization of BMPR1 and BMPR2 that has been described as a requirement to trigger phosphorylation of BMPR1 and subsequent signalling through the SMAD pathway. We investigated the impact on BMPR1A (ALK3, CD292) with BMPR2 (T-ALK) and BMPR1B (ALK6) and with BMPR2 (T-ALK) dimerisation. Dimerisation was measured using the PathHunter® express dimerisation assay (DiscoverX). In this system, U2OS cells are stably transfected with modified human ALK3/ALK6 and BMPR2 intracellularly tagged with inactive enzyme subunits, ProLink™ (PK) or Enzyme Acceptor (EA) respectively. Upon ligand-induced activation, the two receptors dimerize forcing the two enzyme components to complement creating an active enzyme that then hydrolyses a substrate to generate a chemiluminescent signal. Here we used BMP6 to trigger the dimerization event in both cases.

Initially, a BMP6 response curve was established using a range of BMP6 concentration starting at 5 μg/ml final concentration and further doubling dilutions. Eventually, a fixed concentration of 200 ng/ml BMP6 was chosen for analysing the effects of anti-BMP6 antibodies on BMP6 induced receptor dimerisation. The effect of anti-BMP6 monoclonal mouse antibody MAB507 (R&D Systems) and various other anti-BMP6 antibodies on the dimerization was studied at a range of antibody concentrations. Antibody A is an anti-BMP6 antibody comprising VH domains wherein each domain comprises SEQ ID NO: 402; and VL domains, wherein each domain comprises SEQ ID NO: 410. Antibody B is an anti-BMP6 antibody comprising VH domains wherein each domain comprises SEQ ID NO: 418; and VL domains, wherein each domain comprises SEQ ID NO: 426.

FIG. 6A shows the representative result of two experiments carried out for the dimerisation of BMPR1A (ALK3)/BMPR2. All human anti-BMP6 antibodies tested reduced ALK3/BMPR2 receptor dimerisation in a concentration dependent manner leading to a complete inhibition of dimerisation at concentrations greater than 10 nM. The IC50 values in this experiment were 2 nM for the human anti-BMP6 antibodies tested. The murine monoclonal MAB507 (R&D Systems) had a slightly lower IC50 of 3 nM. Whilst not wishing to be bound by any particular theory, the most likely interpretation of this observation is that binding of the anti-BMP6 antibodies to BMP6 prevents BMP6 from interacting with one or both BMP-receptors thus preventing a BMP6-driven dimerization of the type I and type II receptor. However, it is also conceivable that some anti-BMP6 antibodies may bind to BMP6 in a manner that would still allow the interaction of the BMP6-antibody complex with one of those receptors molecules and where then the antibody remains bound to BMP6 on the interaction of the complex with one of the receptors. This bound receptor-antibody complex might then sterically prevent or interfere with the subsequent interaction of this receptor-antibody complex with the other paired receptor thus avoiding an effective dimerization and signal generation in this assay.

FIG. 6B shows the representative result of two experiments carried out for the dimerisation of BMPR1B (ALK6)/BMPR2. Human anti-BMP6 antibodies 1-8 were tested for their ability to impact ALK6/BMPR2 receptor dimerisation in a concentration dependent manner. All antibodies tested lead to a complete inhibition of dimerization. Some of the anti-BMP6 antibodies used here showed only full neutralisation at 100 nM or above. The murine monoclonal MAB507 (R&D Systems) had an IC50 of about 0.2 nM in this assay.

As indicated by The Human Protein Atlas (world wide web.proteinatlas.org/ENSG00000168509-HFE2/cell #rna) U2O2 cells do not express HJV (also known as haemojuvelin, hemojuvelin, HFE2A, HJV, JH and RGMC). Furthermore, we engineered the cell line to express exogenous human BMP receptors, but no engineering was performed to express exogenous HJV. The results, therefore, showed that surprisingly we could inhibit HJV-independent dimerization of BMP receptors by BMP6 using anti-BMP6 antagonists.

### Method

A human ALK3+human BMPR2-expressing cell line created, and a human ALK6+human BMPR2-expressing cell line created (the ALKs were engineered with inactive enzyme subunits, ProLink™ (PK), whereas the BMPR2 was engineered with Enzyme Acceptor (EA). PathHunter® express BMPR1A+BMPR2 and BMPR1B+BMPR2 transfected U2OS cells (DiscoverX #93-1053C3 or #93-1063E3) were resuspended in the provided Cell Plating reagent and 100 μl of the cell suspension added to each well (1×10⁴ cells/well) of a white-walled clear bottom 96-well tissue culture plate (DiscoverX #15-073) and incubated for 24 h at 37 °C. For establishing the appropriate assay window a titration of human BMP6 (Peprotech 120-06) (SEQ ID NO: 2) was prepared starting at a final concentration of 5 μg/ml and diluted 1 in 3 (×11) in Cell Plating Reagent. From the results of this dilution series a fixed final concentration of 200 ng/ml BMP6 was chosen for both experiments. Antibodies were then tested by adding 200 ng/ml hBMP6 final concentration to a serial dilution of anti-BMP6 antibodies to wells of a microtitre plate and incubating for 1 h at room temperature. Following this, 10 μl of this pre-incubated mixture was then added to U2OS cells cultured plate per well and incubated at 37 °C for a further 16 h. Detection reagent was prepared by adding 1 volume Flash Cell Assay Buffer (DiscoverX #30-390) to 4 volumes Flash Substrate Reagent (DiscoverX #10-219). 110 μl of this prepared Detection Mix was added to each well and incubated for 1 h at room temperature in the dark. Plates were read and analysed using an Envision (PerkinElmer) plate reader.

### Example 9

### Linear Epitope Mapping Using Overlapping Peptide Arrays

Linear epitope mapping was carried out using linear peptides covering the entire sequence of mature human BMP6. The BMP6 peptides were elongated with neutral glycine-serine (GSGSGSG) linkers at the C-and N-terminus to avoid generating truncated peptides at the C-and N-terminus. The elongated BMP6 sequence was then converted into overlapping 15 amino acid peptides with an overlap of 14 amino acids. Microarray chips were printed that contained all the overlapping peptides as well as peptide variations for all locations where mouse BMP6 sequence differs from human BMP6 (PEPerPRINT GmbH). The resulting BMP6 peptide microarrays contained 232 different peptides printed in duplicate (464 peptide spots). CL-58838, Antibody A, Antibody B, mab155963 (Abcam) raised with a linear synthetic BMP6 peptide and Morph 6.1 (Acris BM4103) raised with a synthetic peptide of BMP6 (SEQ ID NO: 17; Schluessener et al. 1995).

### Method

Antibodies were used at concentrations of 1 μg/ml, 10 μg/ml and 100 μg/ml, in incubation buffer; incubation for 16 h at 4 °C and shaking at 140 rpm. Species specific secondary antibodies were used: Staining with control antibody mouse monoclonal anti-HA (12CA5) DyLight800 (1:2000) was done simultaneously with secondary antibodies goat anti-human IgG (H+L) DyLight680 (1:5000) and sheep anti-rabbit IgG (H+L) DyLight680 (1:5000). However, to avoid any interference between secondary and control antibody, staining with the mouse control antibody was done after the staining with the secondary antibody goat anti-mouse IgG (H+L) DyLight680.Read out was via the LI-COR Odyssey Imaging System; scanning offset 0.65 mm, resolution 21 μm, scanning intensities of 7/7 (red=700 nm/green=800 nm).

A clear response was seen with Antibody A against a single epitope-like spot pattern formed by adjacent peptides with the consensus motif TLVHLMNPEYV (SEQ ID NO: 8). Equally, a clear response against a single epitope-like spot pattern formed by adjacent peptides was seen with Antibody B with the consensus motif HLMNPEY (SEQ ID NO: 9). A weaker but still clear response against four epitope-like spot patterns formed by adjacent peptides for human or mouse was seen with mab155963 identifying the consensus motifs SASDYNSSELKTA (human; SEQ ID NO: 10), ELKTACRKHELYV (human; SEQ ID NO: 11), GSSDYNGSELKTA (mouse; SEQ ID NO: 12) and ELKTACKKHELYV (mouse/rat; SEQ ID NO: 13). All those motifs exhibited the consensus core motif ELKTA (SEQ ID NO: 14) likely to correspond to a core epitope recognised by this rabbit anti-BMP6 monoclonal and presumably part of the peptide used to generate this monoclonal. A very strong response against two pattern formed by adjacent peptides was seen with Morph6.1 with the consensus motifs QSQDVAR (human; SEQ ID NO: 15) and QSQDVSR (mouse/rat; SEQ ID NO: 16) that only differed by one exchange of amino acids S and A seen between the human and rat/mouse set of BMP6 peptides. These motives are fully contained within the BMP6 peptide sequence used as the immunogen to generate this monoclonal (QSRNRSTQSQDVARVSSASDYNSSELKTAC SEQ ID NO: 17; Schluessener et al. 1995). In contrast to these results, no staining above the noise level of the assay was seen with CL-58838 IgG4 (SEQ ID NO: 116 and SEQ ID NO: 125) even upon significant increased brightness and contrast. However, all positive control linear epitopes used in the assay performed as expected. This outcome is most likely explained by the fact that CL-58838, in contrast to all the antibodies mapped above, binds to a conformationally sensitive or discontinuous epitope that could not be mimicked or represented by the linear antigen-derived peptides in the microarray.

### Example 10

### Western Blot Analysis of Binding to Human BMP6

Since the peptide mapping experiment in Example 9 suggested that CL-58838 was not able to bind overlapping linear peptides generated from the BMP6 primary sequence, the antibody was subjected to Western blotting where human BMP6 was applied to SDS-PAGE under reducing and non-reducing conditions. Since BMP6 is a disulphide linked dimer the reducing condition should create monomers on the gel instead of dimers on the non-reduced gel. The presence of SDS will cause a general unfolding of the protein structure in both cases revealing potential linear epitopes but also remove or at least reduce secondary structures. From the results in Example 9 it was anticipated that CL-58838 might not recognise unfolded BMP6 presented on an SDS-PAGE whereas Antibodies A and B that clearly recognised linear parts of the BMP6 sequence might. FIG. 5 shows the results of this analysis. The Coomassie stained gel clearly shows BMP6 monomers at 18 kDa in the reduced lane and about 32 kDa dimers in the non-reduced lane confirming the formation of monomers on applying reducing conditions. CL-58838 generated a signal on the non-reduced, disulphide linked dimers but not on the reduced monomers. In contrast, Antibody A generated a signal on the dimers and a very defined signal on the BMP6 monomers indicating that the epitope sufficient for binding Antibody A does not require a particular fold or the dimerisation of BMP6. Antibody B gave very comparable results to Antibody A again emphasizing the similarity of the epitope of those two antibodies. These results also indicated that CL-58838 is still able to recognise BMP6 in the presence of SDS but requires the conformational interface of a disulphide linked BMP6 dimer to bind. This conforms with the results seen in Example 9 and may indicate that the epitope for CL-58838 encompasses residues only brought together in the BMP6 dimer and/or that crucial residues mediating binding activity are located close to an intermolecular disulphide link between BMP6 monomers.

### Example 11

### Assessment of Anti-BMP6 Antibodies Following a Single Intravenous Injection (iv) in Normal Rats

Male Wistar rats (250-325 g, n=6/group) were given a single 1 mg/kg iv injection of fully human IgG4 anti-BMP6 antibodies. Two or three control groups were included in each study receiving a single 1 mg/kg iv dose, a hIgG4 isotype control antibody (labelled “isotype”) and one or two additional human IgG anti-BMP6 antibodies (Antibody A and B).

Animals were bled pre-dose at one hour prior to dosing (−1) to give baseline measurements and then at 5 minutes, at 6 and 24 hours and on days 3, 7, 9, 14, 22, 29 and 36. Plasma transferrin saturation (TSAT) was determined for every time point.

These experiments were designed to measure the change in TSAT following a single iv injection of fully human IgG4 anti-BMP6 antibody generated by the invention. A total of 17 antibodies (Tables 4-6 and 7-9) were evaluated in three independent studies according to the experimental procedure outlined above. Tables 12-14 shows the results of these 3 experiments for TSAT.

TABLE 12
TSAT calculated from iron parameters measured in plasma following a single iv injection of
antibodies selected in Table 7. All antibodies and controls dosed at 1 mg/kg as described above

| TSAT | | | | | | | | | | |
|---|---|---|---|---|---|---|---|---|---|---|
| [%] | | | | | | | | | | |
| Time | Isotype | Antibody | | | | | | | | Antibody |
| (Hours) | Control | B | CL-57931 | CL-58851 | CL-66833 | CL-58252 | CL-57945 | CL-58102 | CL-58838 | A |
| −1.00 | 45.52 | 45.42 | 39.08 | 44.45 | 45.75 | 45.07 | 45.72 | 43.98 | 41.45 | 38.58 |
| 0.08 | 46.52 | 44.08 | 39.82 | 42.87 | 43.83 | 43.62 | 46.50 | 44.72 | 46.42 | 41.27 |
| 6.00 | 61.95 | 63.02 | 71.73 | 60.87 | 68.30 | 61.88 | 76.03 | 53.40 | 72.45 | 62.80 |
| 24.00 | 47.88 | 94.95 | 63.72 | 84.38 | 84.43 | 86.17 | 83.95 | 60.38 | 90.47 | 81.73 |
| 72.00 | 52.84 | 86.53 | 64.18 | 83.37 | 66.90 | 76.02 | 82.32 | 50.18 | 87.00 | 78.53 |
| 168.00 | 46.92 | 90.10 | 55.50 | 78.23 | 69.73 | 76.00 | 70.25 | 53.93 | 90.90 | 79.00 |
| 216.00 | 52.68 | 92.38 | 50.47 | 76.12 | 60.35 | 71.72 | 56.55 | 48.23 | 85.85 | 81.95 |
| 336.00 | 47.73 | 89.02 | 42.57 | 50.63 | 55.87 | 50.03 | 45.80 | 47.28 | 71.77 | 79.90 |
| 528.00 | 46.72 | 57.02 | 44.77 | 42.68 | 44.15 | 54.25 | 43.43 | 36.03 | 58.07 | 59.07 |
| 696.00 | 42.17 | 43.00 | 40.85 | 40.60 | 46.03 | 44.38 | 39.07 | 36.75 | 48.50 | 35.70 |
| 864.00 | 41.62 | 34.73 | 39.92 | 36.83 | 47.00 | 43.92 | 34.83 | 36.28 | 47.82 | 33.55 |

TABLE 13
TSAT calculated from iron parameters measured in plasma following a single iv injection of
antibodies selected in Table 8. All antibodies and controls dosed at 1 mg/kg as described above

| TSAT | | | | | | | | | |
|---|---|---|---|---|---|---|---|---|---|
| [%] | | | | | | | | | |
| Time | Isotype | | | | | | | | Antibody |
| (Hours) | Control | CL-75183 | CL-75500 | CL-75506 | CL-75520 | CL-75539 | CL-75565 | CL-75714 | A |
| −1.00 | 40.07 | 42.48 | 41.72 | 46.88 | 39.13 | 46.88 | 41.07 | 44.03 | 37.92 |
| 0.08 | 40.12 | 40.53 | 40.75 | 42.00 | 38.07 | 47.58 | 41.78 | 46.07 | 36.93 |
| 6.00 | 55.57 | 67.85 | 64.60 | 60.35 | 60.13 | 61.02 | 52.45 | 65.10 | 75.82 |
| 24.00 | 40.13 | 47.13 | 52.20 | 51.23 | 49.07 | 50.97 | 45.03 | 74.17 | 98.18 |
| 72.00 | 42.53 | 52.40 | 51.27 | 61.10 | 51.85 | 48.57 | 48.68 | 54.87 | 86.68 |
| 168.00 | 41.15 | 52.70 | 45.07 | 52.53 | 47.28 | 59.72 | 49.62 | 58.25 | 89.30 |
| 216.00 | 41.78 | 48.63 | 50.03 | 52.58 | 49.50 | 50.93 | 45.28 | 52.03 | 95.65 |
| 336.00 | 48.08 | 53.38 | 45.72 | 45.80 | 46.95 | 55.67 | 47.57 | 59.47 | 98.30 |
| 528.00 | 41.50 | 46.00 | 47.33 | 45.17 | 43.83 | 49.33 | 41.33 | 47.67 | 58.17 |
| 696.00 | 40.40 | 40.40 | 44.00 | 42.75 | 40.35 | 48.15 | 44.12 | 46.07 | 38.87 |
| 864.00 | 39.02 | 41.08 | 42.47 | 42.28 | 38.80 | 45.20 | 37.83 | 42.70 | 34.87 |

TABLE 14
TSAT calculated from iron parameters measured in plasma following a single iv injection of
antibodies selected in Table 9. All antibodies and controls dosed at 1 mg/kg as described above

| TSAT | | | | | | |
|---|---|---|---|---|---|---|
| [%] | | | | | | |
| Time | Isotype | Antibody | | | | Antibody |
| (Hours) | Control | B | CL-58722 | CL-58756 | CL-58835 | A |
| −1.00 | 42.37 | 38.98 | 44.98 | 44.05 | 43.32 | 37.73 |
| 0.08 | 43.38 | 36.25 | 44.28 | 45.55 | 46.46 | 38.53 |
| 6.00 | 58.33 | 61.60 | 57.30 | 71.78 | 64.74 | 66.20 |
| 24.00 | 42.77 | 91.95 | 73.35 | 67.38 | 94.58 | 93.78 |
| 72.00 | 45.77 | 79.03 | 62.55 | 51.97 | 92.32 | 89.37 |
| 168.00 | 57.65 | 83.32 | 57.63 | 52.00 | 89.30 | 94.88 |
| 216.00 | 57.57 | 86.27 | 51.82 | 62.38 | 89.64 | 95.62 |
| 336.00 | 49.23 | 79.76 | 51.52 | 52.25 | 80.06 | 91.72 |
| 528.00 | 40.50 | 61.36 | 44.62 | 48.77 | 58.64 | 47.06 |
| 696.00 | 49.28 | 49.50 | 45.72 | 50.37 | 52.56 | 36.10 |
| 864.00 | 33.02 | 37.74 | 39.33 | 41.65 | 43.00 | 33.68 |

Plotted results are shown in FIGS. 8A-8D (for Table 12), FIGS. 8E-8G (for Table 13) and FIGS. 8H-8I (for Table 14). Overall, from all three experiments, the two antibodies that showed the highest effect on TSAT levels in terms of peak increase in TSAT and duration of effect were CL-58838 (FIG. 8A) and CL-58835 (FIG. 8H).

### Example 12

### Dose Response Assessment of CL-55838 Following a Single iv Injection in Normal Rats

Antibody CL-58838 was one of the antibodies that showed a preferable profile in Example 11 and it was therefore of interest to determine the effect on transferrin saturation and duration of effect following IV injection of CL-58838 over a range of doses. Male Wistar rats (250-325 g, n=6/group) were given a single iv injection of 0.3, 1 or 3 mg/kg of CL-58838 fully human IgG4 (SEQ ID NO: 116 and SEQ ID NO: 125). hIgG4 isotype control (labelled “isotype”) and Antibody A were also included receiving a single 1 mg/kg iv dose.

Animals were bleed pre-dose at one hour prior to dosing (−1) to give baseline measurements and then at 5 minutes, at 6 and 24 hours, and on days 3, 7, 9, 14, 22, 29 and 36. Plasma TSAT was determined for every time point (Table 15).

TABLE 15
TSAT calculated from iron parameters measured in plasma following
a single iv injection of antibody CL-58838 at various doses.
Isotype control and Antibody A were dosed at 1 mg/kg only

| | TSAT [%] | | | | |
|---|---|---|---|---|---|
| | | | | | Antibody |
| Time | Isotype | CL-58838 | CL-58838 | CL-58838 | A |
| (Hours) | Control | 0.3 mg/kg | 1 mg/kg | 3 mg/kg | 1 mg/kg |
| −1.00 | 43.13 | 42.43 | 47.72 | 51.47 | 41.35 |
| 0.08 | 40.10 | 40.17 | 43.55 | 46.45 | 43.57 |
| 6.00 | 52.70 | 66.40 | 61.05 | 65.35 | 57.75 |
| 24.00 | 42.98 | 88.18 | 90.23 | 98.30 | 96.95 |
| 72.00 | 39.43 | 73.23 | 82.83 | 96.28 | 90.27 |
| 168.00 | 43.37 | 66.63 | 84.10 | 90.98 | 98.33 |
| 216.00 | 46.45 | 63.78 | 68.23 | 94.50 | 95.97 |
| 336.00 | 46.92 | 50.98 | 84.80 | 95.92 | 96.43 |
| 528.00 | 48.58 | 41.13 | 66.92 | 96.02 | 70.05 |
| 696.00 | 41.12 | 37.47 | 56.45 | 80.48 | 39.87 |
| 864.00 | 40.72 | 34.20 | 46.90 | 67.70 | 31.52 |

FIGS. 9A-9B show the results for this experiment. There was a rapid and strong increase in TSAT with dose dependent peak levels and dose dependent duration of the modulation of TSAT following treatment with CL-58838. 3 mg/kg had the highest and longest duration of effect with TSAT elevated over isotype control for at least 864 h (FIG. 9A). CL-58838 showed a comparable increase in TSAT and duration of effect to Antibody A when compared at the same dose of 1 mg/kg although the peak levels of Antibody A appeared slightly higher but returned to isotype levels by 696 h whereas CL-58838 was still elevated over isotype at this time point (FIG. 9B).

FIGS. 11A-11B show the pharmacokinetic (PK) profile of CL-58838 in this experiment (FIG. 11A) with an increase in peak concentration and duration of exposure with each escalation in dose. When comparing this PK profile to that of Antibody A at the 1 mg/kg dose, CL-58838 showed a comparable peak concentration but an increased duration of exposure (FIG. 11B).

### Example 13

### Dose Response Assessment of CL-55838 Following a Single Subcutaneous (Sc). Injection in Normal Rats

Male Wistar rats, 250-325 g, n=6/group, were given a single sub cutaneous (sc) injection of 0.3, 1 or 3 mg/kg of CL-58838 as a fully human IgG4 anti-BMP6 antibody (SEQ ID NO: 116 and SEQ ID NO: 125). hIgG4 isotype control antibody (labelled “isotype”) and anti-BMP6 Antibodies A and B were also included each receiving a single 1 mg/kg sc dose.

Animals were bleed pre-dose at one hour prior to dosing (−1) to give baseline measurements and then at 5 minutes, at 6 and 24 hours, and on days 3, 7, 9, 14, 22, 29 and 36. Plasma TSAT was determined for every time point (Table 16).

TABLE 16
TSAT calculated from iron parameters measured in plasma following a single sc injection of
antibody CL-58838. Isotype control and antibodies A and B were dosed at 1mg/kg only

| TSAT | Isotype | | | | | |
|---|---|---|---|---|---|---|
| [%] | Control | | | | Antibody | Antibody |
| Time | IgG4 | CL-58838 | CL-58838 | CL-58838 | A | B |
| (Hours) | (IV) | 0.3 mg/kg | 1 mg/kg | 3 mg/kg | 1 mg/kg | 1 mg/kg |
| −1.00 | 43.13 | 41.17 | 42.13 | 42.75 | 43.83 | 44.70 |
| 0.08 | 40.10 | 44.63 | 42.38 | 43.08 | 43.10 | 43.68 |
| 6.00 | 52.70 | 58.75 | 60.53 | 66.07 | 57.83 | 59.28 |
| 24.00 | 42.98 | 79.08 | 87.90 | 97.77 | 94.97 | 88.97 |
| 72.00 | 39.43 | 79.12 | 86.07 | 98.20 | 98.50 | 84.08 |
| 168.00 | 43.37 | 77.48 | 93.37 | 99.10 | 95.72 | 94.82 |
| 216.00 | 46.45 | 64.88 | 88.55 | 98.52 | 98.98 | 88.15 |
| 336.00 | 46.92 | 50.48 | 85.22 | 83.07 | 71.15 | 93.32 |
| 528.00 | 48.58 | 40.67 | 62.65 | 63.60 | 47.47 | 56.90 |
| 696.00 | 41.12 | 40.07 | 50.47 | 54.88 | 39.05 | 45.73 |
| 864.00 | 40.72 | 38.20 | 37.53 | 53.62 | 37.63 | 36.48 |

FIGS. 10A-10B show the results. There was a rapid and strong increase in TSAT at all doses with dose dependent peak levels at 24 to 72 h and a dose dependent duration of the modulation of TSAT following treatment with CL-58838. 3 mg/kg had the longest duration with TSAT elevated over isotype control for at least 864 h (FIG. 10A). CL-58838 showed a very comparable increase in TSAT to Antibody A and B at 1 mg/kg (FIG. 10B). The duration of effect was more similar to Antibody B in this experiment both still elevated over isotype control at 528 h.

FIGS. 11C-11D show the pharmacokinetic (PK) profile of CL-58838 following a single sc dose, with an increase in peak concentration and duration of exposure with each escalation in dose (FIG. 11C). When comparing CL-58838's PK profile to that the anti-BMP-6 antibodies A and B at the | mg/kg dose, CL-58838 showed a comparable peak exposure with an increased duration of exposure (FIG. 11D).

### Example 14

### Assessment of CL-58838 in a PG-PS Rat Model of Anaemia of Chronic Inflammation

The PG-PS rat disease model is a disease model of persistent and long lasting joint inflammation following administration of Group A streptococcal peptidoglycan-polysaccharide (PG-PS). Most of these rats also develop severe anaemia over a two to three week period and the model therefore also represents a model for the inflammation driven functional iron deficiency anaemia observed in many chronic inflammatory diseases, i.e. represents a model for anemia of chronic disease (ACD; (Theurl et al., 2011)).

6-8 week old female Lewis rats were given a single intraperitoneally (ip) injection of 15 mg/kg PG-PS at day-14. On day-1 (week 0) the animals were bled from the tail vein, plasma collected and whole blood taken and used for complete blood count (CBC) analysis. Animals which developed anaemia (haemoglobin level of less than 15 g/dl) as well as an increase in leukocyte count, entered the study. A treatment naïve group, was included in the study for baseline readings (n=5). On day 0, animals were randomised into groups to give a similar mean haemoglobin level and leucocyte count in all of the treated groups (n=6-9). Rats were then treated with either a single sc injection of IgG4 isotype control (n=8), CL-58838 3 mg/kg (n=9), Darbepoietin alpha (EPO) 10 μg/kg (n=8) or a combination of both CL-58838 3 mg/kg and EPO 10 μg/kg (n=7). EPO administration was repeated every week in the groups receiving EPO.

Blood was taken from the tail vein on day-1 (week 0), 7 (week 1), 14 (week 2), 21 (week3) and at cull at day 28 (week 4) to allow further analysis of iron status and anemia.

The experiment was designed to investigate whether CL-58838 was able to positively impact anaemia in the rats compared with untreated control. To determine this effects on haemoglobin, TSAT, whole blood counts and hepcidin levels were measured following a single dose of CL-58838 alone or in combination with erythropoietin (EPO) representing a common treatment option in anaemia of chronic disease.

TABLE 17
Haemoglobin (g/dl), haematocrit (haemocrit, %), mean cell volume (MCV, fL), mean cell
haemoglobin (MCH, pg) and white blood cells (WBC × 10³/μL) measured at 0, 1, 2, 3 and
4 weeks post commencement of treatment. CL-58838 and isotype control were dosed at
3 mg/kg, EPO (Darbepoietin alpha) was dosed at 10 μg/kg.

| | Naïve | | | | | Isotype Control | | | | | CL-58838 | | | | |
|---|---|---|---|---|---|---|---|---|---|---|---|---|---|---|---|
| Week | 0 | 1 | 2 | 3 | 4 | 0 | 1 | 2 | 3 | 4 | 0 | 1 | 2 | 3 | 4 |
| Haemoglobin | 15.4 | 15.8 | 15.7 | 15.2 | 15.4 | 15.6 | 14.7 | 15.8 | 12.4 | 11 | 15.9 | 13.1 | 14.9 | 13.9 | 13.6 |
| Haemocrit | 50 | 48.3 | 515 | 47.8 | 46.7 | 50.5 | 45.6 | 52.3 | 40.3 | 34.2 | 51.1 | 40 | 49.4 | 45.3 | 45.5 |
| MCV | 60 | 59 | 58.5 | 56 | 56.8 | 57.4 | 54.1 | 52.6 | 50.3 | 50.3 | 56.5 | 55.1 | 55 | 51.9 | 52.8 |
| MCH | 18.6 | 19.3 | 17.9 | 17.8 | 18.7 | 17.8 | 17.7 | 16 | 15.6 | 16.4 | 17.6 | 18.1 | 16.7 | 16 | 17 |
| WBC | 7.48 | 7.2 | 6.52 | 5.3 | 4.98 | 11.4 | 16 | 14.9 | 14.8 | 11.1 | 14.3 | 14.4 | 15 | 14 | 10.3 |

| | ESA | | | | | CL-58838 + ESA | | | | |
|---|---|---|---|---|---|---|---|---|---|---|
| Week | 0 | 1 | 2 | 3 | 4 | 0 | 1 | 2 | 3 | 4 |
| Haemoglobin | 15.7 | 13.5 | 15 | 15.4 | 14.1 | 15.8 | 18 | 18.6 | 19 | 23.8 |
| Haemocrit | 51.9 | 42.3 | 51.7 | 51.7 | 45 | 50.3 | 57.3 | 62.6 | 62.9 | 76.9 |
| MCV | 57.3 | 53.9 | 52 | 49.6 | 50.8 | 58.8 | 62.3 | 61.5 | 60.8 | 59.5 |
| MCH | 17.4 | 17.2 | 15.1 | 14.7 | 15.5 | 18.3 | 19.2 | 18 | 18 | 18.5 |
| WBC | 15.3 | 17.3 | 18.3 | 18.4 | 15.6 | 10.7 | 11 | 9.83 | 8.43 | 9.08 |

Table 17 and FIGS. 12A-12F show that treatment with CL-58838 on its own increased TSAT at all four time points compared to animals treated with isotype control (FIG. 12A). Combining CL-58838 with EPO administration elevated the TSAT much further staying elevated at over 50% for the duration of the experiment similar to the TSAT seen in naïve animals. CL-58838 also prevented the drop of haemoglobin seen for the isotype treated animals between week 3 and 4 (FIG. 12B). Combining CL-58838 with EPO administration caused a massive synergistic increase of haemoglobin way beyond the levels seen in naïve animals (FIG. 12B). Similar observations were made for MCH where co-treatment restored the MCH levels observed for naïve animals (FIG. 12C). FIGS. 12 D-12F show the results of circulating hepcidin levels at week 0 (FIG. 12D), week 1 (FIG. 12E) and week 2 (FIG. 12F). At the start of treatment (FIG. 12D) all groups already had vastly elevated hepcidin levels driven by a quick onset of inflammation over naïve animals. At weeks 1 and 2 after starting treatment isotype control animals further increased hepcidin levels presumably due to ongoing inflammation and over production of hepcidin (FIGS. 12E and 12F). Treatment with EPO alone was not able to reduce the increase of hepcidin levels and still increased over time, staying within 20-30% of the isotype control. Treatment with CL-58838 alone had a marked effect on hepcidin levels with an initial substantial decrease over isotype control treated animals and then remaining at around 50% of the hepcidin levels of isotype control treated animals. Treatment with CL-58838 and EPO administration decreased hepcidin levels initially to a similar degree as CL-58838 alone but then maintained or even further reduced hepcidin levels in comparison to isotype, CL-58838 or EPO treated animals. The addition of EPO and CL-58838 together appears to have a synergistic effect on hepcidin levels possibly through the effective stimulation of erythropoiesis in the presence of increased levels of available iron and resulting in a possible down regulation of hepcidin production (Nai et al., 2015).

### Example 15

### Dose Response Assessment of CL-58838 Following a Single iv Dose Injection in Cynomolgus Monkeys

15 male healthy cynomolgus monkeys weighing 2.5-4.0 kg were given a single iv injection of CL-58838 human IgG4 (SEQ ID NO: 116 and SEQ ID NO: 125) at 1, 3 and 10 mg/kg. Anti-BMP6 antibodies A and B were also included as a single iv injection 3 mg/kg.

Animals were bled pre-dose on days −6, −1 and just prior to dosing (0) to give baseline measurements, then at 6/24 hours, and on days 3, 7, 9, 14, 22, 29, 35 and 43. Plasma TSAT and hepcidin levels were measured at every time point to assess the efficacy of CL-58838 at modulating iron metabolism. The values for TSAT at all time points and all groups are shown in Table 18.

TABLE 18
TSAT calculated from iron parameters measured in plasma of
cynomolgus monkeys following a single iv injection of antibody.
Isotype control and antibodies A and B were dosed at 3 mg/kg only

| | TSAT [%] | | | | |
|---|---|---|---|---|---|
| | CL- | CL- | CL- | Antibody | Antibody |
| Time | 58838 | 58838 | 58838 | A | B |
| (Hours) | 1 mg/kg | 3 mg/kg | 10 mg/kg | 3 mg/kg | 3 mg/kg |
| −144 | 30.43 | 44.77 | 31.60 | 34.17 | 42.63 |
| −24 | 33.93 | 39.40 | 33.70 | 36.00 | 39.37 |
| 0 | 40.00 | 36.50 | 49.70 | 42.43 | 49.53 |
| 6 | 35.77 | 27.60 | 37.00 | 34.67 | 33.53 |
| 24 | 59.57 | 80.17 | 81.73 | 85.33 | 90.10 |
| 72 | 62.17 | 74.43 | 94.77 | 94.23 | 89.20 |
| 168 | 69.10 | 79.87 | 93.03 | 77.40 | 75.90 |
| 216 | 57.57 | 82.37 | 93.67 | 89.27 | 82.70 |
| 336 | 41.47 | 65.20 | 78.13 | 71.27 | 67.47 |
| 528 | 53.30 | 57.57 | 68.87 | 63.53 | 73.07 |
| 696 | 56.90 | 52.47 | 68.47 | 61.80 | 66.50 |
| 840 | 59.87 | 58.80 | 57.50 | 70.53 | 67.17 |
| 1032 | 56.70 | 53.97 | 54.13 | 61.03 | 64.93 |

FIGS. 13A-13D show an increase in TSAT and duration of effect following a single iv dose of 3 mg/kg of CL-58838 that was comparable to antibody A and B at the same dose (A). There was a dose dependent increase in overall effect on TSAT, TSAT peak levels and duration of the modulation of TSAT following treatment with CL-58838 at different doses (FIG. 13B). CL-58838 and antibody A and B all showed a similar decrease in plasma hepcidin levels and a comparable duration of effect at 3 mg/kg (FIG. 13C). The decrease in hepcidin following treatment with CL-58838 was maximal at 3 mg/kg and treating with 10 mg/kg showed no further decrease of hepcidin levels (FIG. 13D).

FIGS. 14A-14B show the PK profile of CL-58838 (FIG. 14A) in this experiments with an increased exposure and peak concentration and duration of exposure with each escalation in dose. When comparing the exposure profile of CL-58838 to Antibody A or B at 3 mg/kg all antibodies showed similar profiles but CL-58838 showed a trend for a possibly increased duration of exposure (FIG. 14B).

### Example 16

### Sequence Analysis of CL-58838 and CL-58838-Like Antibodies

Following the results of the in vivo analysis of selected antibodies (Example 11) the sequences of antibodies that referred functional activity were analysed again and compared in light of the fact that only CL-58838 had shown very potent activity of a prolonged elevation of iron levels and TSAT. Since sequences for all antibodies recovered from the Kymouse™ were available, the sequence pool was mined for antibodies with identical VDJ-region (IGHV3-11*01, IGHD6-19*01, IGHJ4*02/IGKV3-20*01, IGKJ1*01) and identical CDRH3 sequences. Several antibodies were identified from the pool of NGS sequences across two immunisation regimes as explained in Example 5 (Table 6) . . . . Eventually three new antibodies were selected for in vivo analysis (shown in bold Table 6 and in Table 9). However, after testing in vivo (Example 11, Table 14) neither CL-58722 or CL-58756 identified by this method showed comparable activity to CL-58838 and only CL-58835 was comparable. This was surprising given the high level of homology between the amino acid sequences in this antibody panel. FIG. 15 shows an alignment of the Vh (IGHV3-11) and Vk (IGKV3-20) regions for all unique antibodies from Table 6 and CL-58838. First to note is that CL-58838 and CL-58835 have identical light chains which are unique to those two antibodies and otherwise only differ by one amino acid in CDRH2. Antibodies that were tested in vivo but did not show comparable potency to CL-58838 or CL-58835 in vivodo not show the germ line Vh changes at residues 75 and 76 (Kabat) to M75 and D76 seen with CL-58838 or CL-58835. No other Vh tested had these mutations or other significant differences that could explain this difference. It is therefore possible that these two mutations may be the major sequence motif mediating the exceptional potency observed for those two antibodies. It cannot be excluded that the unique Vk regions also contributed to the characteristics of CL-58838 and CL-58835 although it is known in the field that the Vh region and the CDRH3 in particular are the often the main contributor to the specificity and potency of antibodies (Xu and Davis, 2000).

Another observation was made when the cross-reactivity with BMP7 was considered. Antibodies that showed a degree of cross-reactivity with BMP7 in this panel were CL-58680, CL-58679 and CL-58680. Sequence differences in the Vh domain cannot be attributed to this effect since they also occur in other clones. However, in the Vk domain position 52 and 53 (Kabat) are not changed from germline IGKV3-20 for the clones that cross-reacted with BMP7 whereas all the clones that did not cross-react with BMP7 have one or both of these residues changed.

The in vitro potency of those antibodies was comparable when tested in a head to head competition experiment in a homogeneous time resolved FRET (HTRF) assay where biotinylated CL-58838 detected with streptavidin-cryptate is binding to BMP6 labelled with 647 (FIG. 16). This interaction of labelled CL-58838 with labelled human BMP6 was then competed by unlabelled antibodies tested in this experiment. CL-58838 competed fully with labelled CL-58838 as expected. CL-58722 and CL-58756 showed somewhat lower ability to compete with labelled CL-58838 possibly reflecting also some impact of the sequence changes on the affinity for BMP6. This analysis surprisingly demonstrated that although the antibodies selected for in vivo analysis from this series (with identical VDJ usage and identical CDRH3) demonstrated very comparable ability to bind and neutralise human BMP6 and all compete with CL-58838 for binding to BMP6 yet still differed significantly in their in vivo potency.

### Example 17

### Assessment of the ESA-Sparing Capacity of CL-58838 in a PG-PS Rat Model of Anaemia of Chronic Inflammation

As detailed in Example 14, the PG-PS rat disease model is a well-established model to study the anaemia of chronic inflammation.

The aim of this study was to test if the anti-BMP-6 IgG4 antibody CL-58838 (SEQ ID NO: 116 and SEQ ID NO: 125)) in combination with Darbepoetin alpha (EPO) can reduce the EPO doses needed to effectively restore haematological parameters using this model compared to EPO monotherapy.

Method 6-8 week old female Lewis rats were given a single intraperitoneally (ip) injection of 15 mg/kg PG-PS at timepoint “W-2” and after two weeks (i.e. timepoint “W0”) animals were bled from the tail vein to perform complete blood count analysis. Only if rats had an increased granulocyte count at timepoint W0, they were included in this experiment (see FIG. 17A). An untreated naïve group was included in the study for baseline readings (Group I, see Table 19). In addition, Haemoglobin (Hgb) values of included rats were examined. If the Hgb-value was <13.5 g/dL the rat was allocated into a specific treatment group (II-IV, see Table 19). If the Hgb value was >13.5 g/dL rats were not allocated to a specific treatment group and re-analysed the next week (see FIG. 17 B). At all time points plasma was collected for further analysis. After allocation of the animals to one respective treatment group, rats in Group II were treated with a single s.c. injection of IgG4 control antibody, rats in Group III were treated with Darbepoetin alfa 10 μg/kg and rats in Group IV received a combination of CL-58838 3 mg/kg and Darbepoetin alfa 10 μg/kg (see Table 19).

TABLE 19
Treatment groups to assess the ESA-sparing capacity
of CL-58838 for treatment of ACD.

| | |
|---|---|
| Group I | Naive control rats |
| Group II | ACD rats receiving IgG4 control antibody [3 mg/kg] s.c. |
| Group III | ACD rats receiving Darbepoetin alfa [10 μg/kg] s.c |
| Group IV | ACD rats receiving CL-58838 [3 mg/kg] |
| | and Darbepoetin alfa [10 μg/kg] |

After the initial treatment, administration of CL-58838 (Group IV) or IgG4 control (Group II), was repeated every three weeks. In contrast, any further EPO administration was dependent on the Hgb-value, which was evaluated in each individual rat every week throughout the whole study. As shown in FIG. 17 B, EPO was only administered if the Hgb-value in treatment groups III and IV was below the average Hgb value of the naïve control rats (Group I). We choose this experimental set up to mimic real life clinical setting in humans as CKD patients only receive EPO dependent on a certain pre-defined Hgb-level.

Blood was taken every week (i.e. timepoint “W0-W6”) from the tail vein and at cull at “W7” to allow further analysis of iron status and anaemia.

From “W0” onwards, rats were allocated to one of the treatment groups according to the criteria described above. After Week 3 all rats were allocated to one of the treatment group and treatment could be started. Of importance, Hgb values were evenly distributed among the groups at the day of 1^{st} treatment (FIG. 17 C). Details on the numbers of rats and the time point of treatment initiation is shown in Table 20.

TABLE 20
Rats listed according to their time point of inclusion in the experiment.

| | | Number |
|---|---|---|
| | | (included in |
| Group | Treatment | week 0/1/2/3) |
| Group I | Naive controls | 5 (5/0/0/0) |
| Group II | ACD/IgG4 control antibody [3 mg/kg] | 7 (2/2/2/1) |
| Group III | ACD/Darbepoetin alfa [10 μg/kg] | 10 (1/5/2/1) |
| Group IV | ACD/CL-58838 [3 mg/kg] | 11 (1/5/3/2) |
| | and Darbepoetin alfa [10 μg/kg] | |

### Results

As outlined above, rats were allocated into a treatment group if their Hgb value was <13.5 g/dL. ACD rats treated with the isotype control antibody stayed anaemic throughout the experiment. Treatment with EPO alone could restore Hgb-levels to near normal values. However, the combination of CL-58838 and EPO showed a synergistic effect on Hgb-values. Already one week after treatment initiation, Hgb-values were normalised and further increased over time (FIG. 17 C). In addition, the mean corpuscular volume (MCV) and mean corpuscular haemoglobin (MCH) as sensitive markers for iron availability were normalised in ACD rats treated with a combination of CL-58838 and EPO, while ACD control rats and rats treated with EPO only stayed microcytic and hypochromic throughout the whole experimental period (FIGS. 17 D and 17E).

In addition, to evaluate if combination therapy has an EPO-sparing effect, the amount of EPO, that was administered in rats of treatment group III and IV was monitored. The following calculations are based on the additional EPO doses, thus excluding the EPO dose that was administered for the 1^{st} treatment:

In total, a maximum of 40 (ACD/Darbepoetin alfa) and 44 (ACD/Darbepoetin alfa+CL-58838) EPO administrations would have been theoretically possible after 4 weeks of treatment had all rats fallen below the defined threshold of the average Hgb value of the naïve control rats. Sticking to our criteria explained above and outlined in FIG. 17B, 21 out of 40 possible doses had to be administered to rats of Group III. In contrast, only 8 additional EPO doses had to be applied to rats of Group IV out of the 44 possible EPO administrations. In other words, while EPO-treated rats “consumed” 53% of all possible EPO doses, the combination group only needed only 18% to maintain the Hb values above the cut off (FIG. 17 F). This clearly shows that the combination therapy can correct the Hgb-value much more efficiently than EPO monotherapy. Consequently, addition of CL-58838 is capable to result in an EPO-sparing effect.

### Example 18

### Assessment of CL-58838 in a Mouse Model of Chronic Kidney Disease

Beyond the rat model described in Examples 14 and 17, the human IgG4 anti-BMP-6 antibody CL-58838 (SEQ ID NO: 116 and SEQ ID NO: 125) was also tested in a murine chronic kidney disease model (CKD). Kidney damage was induced via a special diet containing 0.2% Adenine (Akchurin et al., 2016). The aim of this study was to test the effects of CL-58838 as a monotherapy or in combination with Darbepoetin alfa (EPO) in this disease model.

### Method

3-week-old male C57BL/6N mice were fed a special diet containing 0.2% adenine, 0.9% phosphate and 30 mg iron (referred to as Adenine diet). Untreated control mice (n=7) were fed a diet containing 30 mg Fe without Adenine.

At timepoint “Week 0” CKD animals were randomised into the different treatment groups (for details see Table 21). Thus, Group II was treated with a single s.c. injection of IgG4 control [3 mg/kg] (n=5), Group III received CL-58838 [3 mg/kg] (n=6), Group IV was treated with Darbepoetin alfa [10 μg/kg] (n=6) and Group V received a combination of both CL-58838 [3 mg/kg] and Darbepoetin alfa [10 μg/kg] (n=6). An untreated naïve group, was included in the study for baseline readings (n=7). EPO treatment was repeated weekly, i.e. mice were treated 4 times with EPO. CL-58838 administration was done every second week, i.e. mice received two injections of the drug. The experiment was terminated after 4 weeks of treatment.

The experimental setup is shown in FIG. 18A.

TABLE 21
Treatment groups to assess CL-58838 in a mouse
model of chronic kidney disease

| | |
|---|---|
| Group I | Healthy controls |
| Group II | CKD mice receiving IgG4 control antibody [3 mg/kg] i.p. |
| Group III | CKD mice receiving CL-58838 [3 mg/kg] i.p. |
| Group IV | CKD mice receiving Darbepoetin alfa [10 μg/kg] i.p. |
| Group V | CKD mice receiving CL-58838 [3 mg/kg] |
| | and Darbepoetin alfa [10 μg/kg] i.p. |

### Results

4 weeks after the 1^{st} treatment, mice were analysed. Monotherapy with either CL-58838 or EPO could slightly improve Hgb values, however, combination therapy normalised Hgb values to baseline values (FIG. 18 B). Moreover, CL-58838 treated animals had significantly higher mean corpuscular volume (MCV) and mean corpuscular haemoglobin (MCH) values than untreated or EPO only treated mice. Of importance, MCV and MCH were completely normalised in double-treated animals, suggesting an improved supply of iron for erythroid progenitors of these treated animals (FIGS. 18 C and 18 D). As CL-58838 modulates hepcidin expression via targeting BMP6, we took a detailed look into genes, proteins and parameters related to iron metabolism in these animals. As shown in FIG. 18 E, hepatic Hamp expression levels were significantly reduced in animals receiving CL-58838 monotherapy or in combination with Darbepoetin alfa. Plasma iron levels and consequently transferrin saturation (TSAT) values were decreased in CKD control animals. EPO as well as CL-58838 monotherapy led to a slight increase of iron levels and TSAT. Again, combination therapy caused the strongest increase in plasma iron levels (FIG. 18 F). In accordance therewith, also TSAT values significantly increased in animals receiving the double treatment compared to animals treated with EPO alone (FIG. 18 G). In conclusion these data clearly show that iron is efficiently mobilised, bound to Tf, being incorporated into haemoglobin of red blood cells in the bone marrow.

### Example 19

### Assessment of ESA-Sparing Capacity of CL-58838 in a Mouse Model of Chronic Kidney Disease

As detailed in Example 18, CKD was induced in male mice via a diet containing Adenine, causing kidney damage and leading to anaemia after 8 weeks. The aim of this study was to test if
1) combining therapy of CL-58838 and EPO (Darbepoetin alfa) could lead to a better overall response and a potential reduction of the dose of EPO.
2) an EPO dose that on its own had no effect on improving Hgb outcome could be made effective if combined with CL-58838.

### Method

3-week-old male C57BL/6N mice were fed a special diet containing 0.2% adenine, 0.9% phosphate and 30 mg iron (referred to as Adenine diet). At “Week 0” CKD animals were randomised into the different treatment groups (for details see Table 22 and FIG. 18A) and treatment was started. 4 weeks after the 1^{st} treatment, mice were analysed.

TABLE 22
Treatment groups to assess

| Treatment group | Darbepoetin alfa [μg/kg] | CL-58838 [mg/kg] |
|---|---|---|
| Group I | 0 | 0 |
| Group II | 1 | 0 |
| Group III | 1 | 0.1 |
| Group IV | 0.01 | 1 |
| Group V | 0.1 | |
| Group VI | 1 | |
| Group VII | 0.01 | 10 |
| Group VIII | 0.1 | |
| Group IX | 1 | |

### Results

Treatment of mice suffering from CKD with the maximal EPO dose in this study (1 μg/kg) could not improve anaemia. However, if 1 μg/kg EPO was combined with CL-58838, irrespective of the CL-58838 dose [0.1 mg/kg, 1 mg/kg and 10 mg/kg], anaemia could be significantly ameliorated (FIG. 19A). Effects were more pronounced with the two higher doses of CL-58838. These results clearly show that an EPO dose, which has no effect on Hgb levels on its own, if combined with CL-58838, can be made effective.

Vice versa, our experimental setup allowed to evaluate if combination therapy can lead to using lower doses of EPO in a combination treatment (i.e. ESA-sparing). In this respect, a dose of 10 mg/kg CL-58838 was capable to significantly increase Hb levels if combined with 0.1 μg/kg EPO, which is 10-fold lower than the EPO dose used alone, and even 100-fold lower than the dose used in Example 18 (FIG. 19 B).

Moreover, mean corpuscular volume (MCV), being a surrogate for iron availability for erythrocytes, was dose-dependently increased (FIG. 19 C). In other words, the higher the dose of administered CL-58838, the better was the supply of erythrocytes with iron, which is reflected as higher MCV values. As it is well established that iron in the form of saturated transferrin (TSAT) represents the major iron-delivery pathway for developing erythrocytes, we also evaluated this parameter. As shown in FIG. 19D TSAT was significantly increased in mice receiving 1 μg/kg EPO in combination with 1 mg/kg and 10 mg/kg CL-58838, compared to mice receiving EPO alone.

In addition, even the lowest EPO dose (0.01 μg/kg) in combination with 1 mg/kg of CL-58838, which did not lead to a significant increase in Hb (not shown), did lead to a higher MCV values, indicating better red blood cell quality (FIG. 19 E).

Anti-BMP6 via CL-58838 treatment targets at lowering hepcidin levels. Thus, hepcidin levels (by measuring Hamp mRNA expression levels in the liver, which are known to correlate well with plasma hepcidin levels), were analysed. In general, there was a clear trend towards lower Hamp expression levels in all double-treated animals, except mice being in Group III, who received the lowest herein tested CL-58838 dose [0.1 mg/kg] (FIG. 19 F). Of interest, animals receiving the same CL-58838 dose but different doses of EPO (ie. treatment groups IV—VI and VII-IX), revealed a tendency towards higher Hamp expression levels with higher EPO doses. As Hb levels also dose-dependently increased, higher Hamp expression levels must be interpreted in view of the crosstalk between iron homeostasis and erythropoiesis. For example, in case of sufficient Hb levels, Hamp expression is no longer as strongly repressed by signals generated by developing erythrocytes, such as Erfe (Kautz et al., 2014).

### REFERENCES

Akchurin, O., Sureshbabu, A., Doty, S. B., Zhu, Y.-S., Patino, E., Cunningham-Rundles, S., Choi, M. E., Boskey, A., Rivella, S., 2016. Lack of hepcidin ameliorates anemia and improves growth in an adenine-induced mouse model of chronic kidney disease. Am. J. Physiol.-Ren. Physiol. 311, F877-F889.
Andriopoulos, B., Corradini, E., Xia, Y., Faasse, S. A., Chen, S., Grgurevic, L., Knutson, M. D., Pietrangelo, A., Vukicevic, S., Lin, H. Y., Babitt, J. L., 2009. BMP6 is a key endogenous regulator of hepcidin expression and iron metabolism. Nat. Genet. 41, 482-7.
Casanovas, G., Banerji, A., D'Alessio, F., Muckenthaler, M. U., Legewie, S., 2014. A Multi-Scale Model of Hepcidin Promoter Regulation Reveals Factors Controlling Systemic Iron Homeostasis. PLoS Comput. Biol. 10.
Kautz, L., Jung, G., Nemeth, E., Ganz, T., 2014. Erythroferrone contributes to recovery from anemia of inflammation. Blood 124, 2569-74.
Mayeur, C., Lohmeyer, L. K., Leyton, P., Kao, S. M., Pappas, A. E., Kolodziej, S. A., Spagnolli, E., Yu, B., Galdos, R. L., Yu, P. B., Peterson, R. T., Bloch, D. B., Bloch, K. D., Steinbicker, A. U., 2014. The type I BMP receptor Alk3 is required for the induction of hepatic hepcidin gene expression by interleukin-6. Blood 123, 2261-2268.
Nai, A., Lidonnici, M. R., Rausa, M., Mandelli, G., Pagani, A., Silvestri, L., Ferrari, G., Camaschella, C., 2015. The second transferrin receptor regulates red blood cell production in mice. Blood 125, 1170-1179.
Theurl, I., Schroll, A., Sonnweber, T., Nairz, M., Theurl, M., Willenbacher, W., Eller, K., Wolf, D., Seifert, M., Sun, C. C., Babitt, J. L., Hong, C. C., Menhall, T., Gearing, P., Lin, H. Y., Weiss, G., 2011. Pharmacologic inhibition of hepcidin expression reverses anemia of chronic inflammation in rats. Blood 118, 4977-4984.
Xia, Y., Babitt, J. L., Sidis, Y., Chung, R. T., Lin, H. Y., 2008. Hemojuvelin regulates hepcidin expression via a selective subset of BMP ligands and receptors independently of neogenin. Blood 111, 5195-5204.
Xu, J. L., Davis, M. M., 2000. Diversity in the CDR3 region of V(H) is sufficient for most antibody specificities. Immunity 13, 37-45.
Yusa, K., Zhou, L., Li, M. A., Bradley, A., Craig, N. L., 2011. A hyperactive piggyBac transposase for mammalian applications. Proc. Natl. Acad. Sci. U.S.A 108, 1531-6.

SEQUENCES:

| Seq | | | | |
|---|---|---|---|---|
| ID | | | | |
| No: | | | Description | Sequence |
| 1 | Human | | Amino acid sequence Uniprot ID | MPGLGRRAQWLCWWWGLLCSCCGPPPLRPPLPAAAAAAAGGQLLGDGGSPGRTEQ |
| | BMP6 | | number: 22004 | PPPSPQSSSGFLYRRLKTQEKREMQKEILSVLGLPHRPRPLHGLQQPQPPALRQQEEQQ |
| | | | (leader sequence in italics; | QQQQLPRGEPPPGRLKSAPLFMLDLYNALSADNDEDGASEGERQQSWPHEAASSSQR |
| | | | propeptide underlined; mature | RQPPPGAAHPLNRKSLLAPGSGSGGASPLTSAQDSAFLNDADMVMSFVNLVEYDKEFS |
| | | | protein bold) | PRQRHHKEFKFNLSQIPEGEVVTAAEFRIYKDCVMGSFKNQTFLISIYQVLQEHQHRDSD |
| | | | | LFLLDTRVVWASEEGWLEFDITATSNLWVVTPQHNMGLQLSVVTRDGVHVHPRAAGL |
| | | | | VGRDGPYDKQPFMVAFFKVSEVHVRTTRSASSRRRQQSRNRSTQSQDVARVSSASDY |
| | | | | NSSELKTACRKHELYVSFQDLGWQDWIIAPKGYAANYCDGECSFPLNAHMNATNHA |
| | | | | IVQTLVHLMNPEYVPKPCCAPTKLNAISVLYFDDNSNVILKKYRNMVVRACGCH |
| 2 | Human | Recombinant | Amino acid sequence Peprotech | VSSASDYNSSELKTACRKHELYVSFQDLGWQDWIIAPKGYAANYCDGECSFPLNAHMN |
| | BMP6 | protein | Catalog Number: 120-06 | ATNHAIVQTLVHLMNPEYVPKPCCAPTKLNAISVLYFDDNSNVILKKYRNMV |
| | | | | VRACGCH |
| 3 | Human | Recombinant | Amino acid sequence R&D | QQSRNRSTQSQDVARVSSASDYNSSELKTACRKHELYVSFQDLGWQDWIIAPKGYAAN |
| | BMP6 | protein | Systems | YCDGECSFPLNAHMNATNHAIVQTLVHLMNPEYVPKPCCAPTKLNAISVLYFDDNSNVI |
| | | | Catalog Number: 507-BP | LKKYRNMVVRACGCH |
| 4 | Mouse | | Amino acid sequence Uniprot ID | MPGLGRRAQWLCWWWGLLCSCGPPPLRPPLPVAAAAAGGQLLGAGGSPVRAEQPP |
| | BMP6 | | number: P20722 | PQSSSSGFLYRRLKTHEKREMQKEILSVLGLPHRPRPLHGLQQPQPPVLPPQQQQQQQ |
| | | | (leader sequence in italics; | QQQTAREEPPPGRLKSAPLFMLDLYNALSNDDEEDGASEGVGQEPGSHGGASSQLRQ |
| | | | propeptide underlined; mature | PSPGAAHSLNRKSLLAPGPGGGASPLTSAQDSAFLNDADMVMSFVNLVEYDKEFSPHQ |
| | | | protein bold) | RHHKEFKFNLSQIPEGEAVTAAEFRVYKDCVVGSFKNQTFLISIYQVLQEHQHRDSDLFLL |
| | | | | DTRVVWASEEGWLEFDITATSNLWVVTPQHNMGLQLSVVTRDGLHVNPRAAGLVGR |
| | | | | DGPYDKQPFMVAFFKVSEVHVRTTRSASSRRRQQSRNRSTQSQDVSRGSGSSDYNGS |
| | | | | ELKTACKKHELYVSFQDLGWQDWIIAPKGYAANYCDGECSFPLNAHMNATNHAIVQ |
| | | | | TLVHLMNPEYVPKPCCAPTKLNAISVLYFDDNSNVILKKYRNMVVRACGCH |
| 5 | Mouse | Recombinant | Amino acid sequence R&D | SASSRRRQQSRNRSTQSQDVSRGSGSSDYNGSELKTACKKHELYVSFQDLGWQDWIIA |
| | BMP6 | protein | Systems (based on UniProt ID | PKGYAANYCDGECSFPLNAHMNATNHAIVQTLVHLMNPEYVPKPCCAPTKLNAISVLYF |
| | | | P20722) | DDNSNVILKKYRNMVVRACGCH |
| | | | Catalog Number: 6325-BM | |
| 6 | | Forward | Nucleotide sequence | AAAAAAACTAGTAAATGGCCCCATGTGGCCCCCGCCTTGTCTGC |
| | | primer | | |
| 7 | | Reverse | Nucleotide sequence | TTTTTTCTCGAGCTGTCTGGCTGTCCCACTGCTGGGTCTTGAGCTT |
| | | primer | | |
| 8 | | Peptide | Amino acid sequence | TLVHLMNPEYV |
| 9 | | Peptide | Amino acid sequence | HLMNPEY |
| 10 | | Peptide | Amino acid sequence | SASDYNSSELKTA |
| 11 | | Peptide | Amino acid sequence | ELKTACRKHELYV |
| 12 | | Peptide | Amino acid sequence | GSSDYNGSELKTA |
| 13 | | Peptide | Amino acid sequence | ELKTACKKHELYV |
| 14 | | Peptide | Amino acid sequence | ELKTA |
| 15 | | Peptide | Amino acid sequence | QSQDVAR |
| 16 | | Peptide | Amino acid sequence | QSQDVSR |
| 17 | | Peptide | Amino acid sequence | QSRNRSTQSQDVARVSSASDYNSSELKTAC |
| 18 | CL-66833 | CDRH1(IMGT) | Amino acid sequence of CDRH1 | GYTFTNHA |
| | | | using IMGT | |
| 19 | CL-66833 | CDRH2(IMGT) | Amino acid sequence of CDRH2 | INAGNGKT |
| | | | using IMGT | |
| 20 | CL-66833 | CDRH3(IMGT) | Amino acid sequence of CDRH3 | TRRVYGESYDH |
| | | | using IMGT | |
| 21 | CL-66833 | CDRH1 | Amino acid sequence of CDRH1 | NHAIH |
| | | (KABAT) | using Kabat | |
| 22 | CL-66833 | CDRH2 | Amino acid sequence of CDRH2 | WINAGNGKTDYSQNFQG |
| | | (KABAT) | using Kabat | |
| 23 | CL-66833 | CDRH3 | Amino acid sequence of CDRH3 | RVYGESYDH |
| | | (KABAT) | using Kabat | |
| 24 | CL-66833 | Heavy chain | Amino acid sequence of V_{H} | QVQFVQSGAEVKSPGASVKVSCKASGYTFTNHAIHWVRQAPGQRLEWMGWINAGN |
| | | variable | | GKTDYSQNFQGRVIITRDTSANTAYMSLSSLTSEDTAFYYCTRRVYGESYDHWGQGTLV |
| | | region | | TVSS |
| 25 | CL-66833 | Heavy chain | Nucleotide sequence of V_{H} | CAGGTCCAGTTTGTGCAGTCTGGGGCTGAGGTGAAGAGTCCTGGGGCCTCTGTGAA |
| | | variable | | GGTTTCCTGCAAGGCTTCTGGATACACCTTCACA AATCATGCTA TACATTGGGT GC |
| | | region | | GCCAGGCC CCCGGACAAA GGCTTGAGTG GATGGGATGG ATCAACGCTGGCAATG |
| | | | | GTAAAACTGATTATTCACAGAACTTCCAGGGCAGAGTCATCATTACCAGGGACACAT |
| | | | | CCGCGAACACAGCCTACATGTCCCTGAGCAGCCTGACATCTGAGGACACGGCTTTTT |
| | | | | ATTACTGTACTAGAAGGGTCTACGGTGAATCGTATGACCA |
| | | | | CTGGGGCCAGGGAACCCTGGTCACCGTCTC CTCA |
| 26 | CL-66833 | Full heavy | Amino acid sequence heavy | QVQFVQSGAEVKSPGASVKVSCKASGYTFTNHAIHWVRQAPGQRLEWMGWINAGN |
| | | chain | chain | GKTDYSQNFQGRVIITRDTSANTAYMSLSSLTSEDTAFYYCTRRVYGESYDHWGQGTLV |
| | | sequence | | TVSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 27 | CL-66833 | CDRL1(IMGT) | Amino acid sequence of CDRL1 | QGIRNN |
| | | | using IMGT | |
| 28 | CL-66833 | CDRL2(IMGT) | Amino acid sequence of CDRL2 | AAS |
| | | | using IMGT | |
| 29 | CL-66833 | CDRL3(IMGT) | Amino acid sequence of CDRL3 | LQHQIYPWT |
| | | | using IMGT | |
| 30 | CL-66833 | CDRL1 | Amino acid sequence of CDRL1 | RASQGIRNNLG |
| | | (KABAT) | using KABAT | |
| 31 | CL-66833 | CDRL2 | Amino acid sequence of CDRL2 | AASSLQS |
| | | (KABAT) | using KABAT | |
| 32 | CL-66833 | CDRL3 | Amino acid sequence of CDRL3 | LQHQIYPWT |
| | | (KABAT) | using KABAT | |
| 33 | CL-66833 | Light chain | Amino acid sequence of V_{L} | DIQMTQSPSSLSASVGDRVTITCRASQGIRNNLGWYQQKPGKAPKRLIYAASSLQSGVP |
| | | variable | | SRFSGSGSGTEFTLTISSLQPEDVAIYFCLQHQIYPWTFGQGTKVETK |
| | | region | | |
| 34 | CL-66833 | Light chain | Nucleic acid sequence of V_{L} | GACATCCAGA TGACCCAGTC TCCATCCTCC CTGTCTGCAT CTGTAGGAGA CAGAG |
| | | variable | | TCACC ATCACTTGCC GGGCAAGTCA |
| | | region | | GGGCATTAGA AATAATTTAG GCTGGTATCA GCAGAAACCA GGGAAAGCCC CTAA |
| | | | | GCGCCT GATCTATGCT GCATCCAGTT |
| | | | | TGCAAAGTGG GGTCCCATCA AGGTTTAGCG GCAGTGGATC TGGGACAGAA TTCA |
| | | | | CTCTCA CAATCAGCAG CCTGCAGCCT |
| | | | | GAAGATGTTG CAATTTATTT CTGTCTACAA CATCAAATTT ACCCGTGGAC GTTCGG |
| | | | | CCAA GGGACCAAGG TGGAAACCAA |
| | | | | A |
| 35 | CL-66833 | Full light | Amino acid sequence light chain | DIQMTQSPSSLSASVGDRVTITCRASQGIRNNLGWYQQKPGKAPKRLIYAASSLQSGVP |
| | | chain | | SRFSGSGSGTEFTLTISSLQPEDVAIYFCLQHQIYPWTFGQGTKVETK |
| | | sequence | | RTVAAPSVFIFPPSDEQLKSGTASVVCLLNNFYPREAKVQWKVDNALQSGNSQESVTEQ |
| | | | | DSKDSTYSLSSTLTLSKADYEKHKVYACEVTHQGLSSPVTKSFNRGEC |
| 36 | CL-57931 | CDRH1(IMGT) | Amino acid sequence of CDRH1 | GYTFTSYA |
| | | | using IMGT | |
| 37 | CL-57931 | CDRH2(IMGT) | Amino acid sequence of CDRH2 | ISAANGNT |
| | | | using IMGT | |
| 38 | CL-57931 | CDRH3(IMGT) | Amino acid sequence of CDRH3 | ARRKLWSPFDI |
| | | | using IMGT | |
| 39 | CL-57931 | CDRH1 | Amino acid sequence of CDRH1 | SYALH |
| | | (KABAT) | using Kabat | |
| 40 | CL-57931 | CDRH2 | Amino acid sequence of CDRH2 | WISAANGNTDYSWKFQG |
| | | (KABAT) | using Kabat | |
| 41 | CL-57931 | CDRH3 | Amino acid sequence of CDRH3 | RKLWSPFDI |
| | | (KABAT) | using Kabat | |
| 42 | CL-57931 | Heavy chain | Amino acid sequence of V_{H} | QVQLVQSGAEVQKPGASVKVSCKASGYTFTSYALHWVRQAPGQRLEWLGWISAANG |
| | | variable | | NTDYSWKFQGRVTLTRDTSANTVYMELNSLTSEDSAVYYCARRKLWSPFDIWGQGTLV |
| | | region | | TVSS |
| 43 | CL-57931 | Heavy chain | Nucleotide sequence of V_{H} | CAGGTCCAACTTGTGCAGTCTGGGGCTGAGGTGCAGAAGCCTGGGGCCTCAGTGAA |
| | | variable | | GGTTTCCTGCAAGGCTTCTGGATACACCTTCACTTCATATGCTTTGCATTGGGTGCG |
| | | region | | CAGGCCCCCGGACAAAGGCTTGAGTGGCTGGGATGGATCAGCGCTGCCAATGGTAA |
| | | | | CACAGATTATTCATGGAAGTTCCAGGGCAGAGTCACCCTTACCAGGGACACATCCGC |
| | | | | AAACACAGTCTACATGGAACTGAACAGTCTGACATCTGAGGACTCGGCTGTGTATTA |
| | | | | CTGTGCGAGAAGGAAACTATGGTCTCCTTTTGATATCTGGGGCCAAGGGACATTGG |
| | | | | TCACCGTCTCTTCA |
| 44 | CL-57931 | Full heavy | Amino acid sequence heavy | QVQLVQSGAEVQKPGASVKVSCKASGYTFTSYALHWVRQAPGQRLEWLGWISAANG |
| | | chain | chain | NTDYSWKFQGRVTLTRDTSANTVYMELNSLTSEDSAVYYCARRKLWSPFDIWGQGTLV |
| | | sequence | | TVSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 45 | CL-57931 | CDRL1(IMGT) | Amino acid sequence of CDRL1 | QSLTNSF |
| | | | using IMGT | |
| — | CL-57931 | CDRL2(IMGT) | Amino acid sequence of CDRL2 | GAF |
| | | | using IMGT | |
| 47 | CL-57931 | CDRL3(IMGT) | Amino acid sequence of CDRL3 | QYYGTSPWT |
| | | | using IMGT | |
| 48 | CL-57931 | CDRL1 | Amino acid sequence of CDRL1 | WASQSLTNSFLA |
| | | (KABAT) | using KABAT | |
| 49 | CL-57931 | CDRL2 | Amino acid sequence of CDRL2 | GAFSRAT |
| | | (KABAT) | using KABAT | |
| 50 | CL-57931 | CDRL3 | Amino acid sequence of CDRL3 | QYYGTSPWT |
| | | (KABAT) | using KABAT | |
| 51 | CL-57931 | Light chain | Amino acid sequence of V_{L} | EIVLTQSPGTLSLSPGERATLSCWASQSLTNSFLAWYRQKPGQAPRLLISGAFSRATDIPD |
| | | variable | | RISGSGSGTDFTLTINRLEPEDFAVYYCQYYGTSPWTFGQGTKVEIK |
| | | region | | |
| 52 | CL-57931 | Light chain | Amino acid sequence of V_{L} | GAAATTGTGTTGACGCAGTCTCCAGGCACCCTGTCTTTGTCTCCAGGGGAAAGAGCC |
| | | variable | | ACCCTCTCCTGTTGGGCCAGTCAAAGTCTTACCAACAGTTTCTTAGCCTGGTACCGGC |
| | | region | | AGAAACCTGGCCAGGCTCCCAGGCTCCTCATCTCTGGTGCATTCAGCAGGGCCACTG |
| | | | | ACATCCCAGACAGGATCAGTGGCAGTGGATCTGGGACAGACTTCACTCTCACCATCA |
| | | | | ACAGACTGGAGCCTGAAGATTTTGCAGTGTATTACTGTCAGTACTATGGTACCTCAC |
| | | | | CGTGGACGTTCGGCCAAGGGACCAAGGTGGAAATCAAA |
| 53 | CL-57931 | Full light | Amino acid sequence light chain | EIVLTQSPGTLSLSPGERATLSCWASQSLTNSFLAWYRQKPGQAPRLLISGAFSRATDIPD |
| | | chain | | RISGSGSGTDFTLTINRLEPEDFAVYYCQYYGTSPWTFGQGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 54 | CL-57945 | CDRH1(IMGT) | Amino acid sequence of CDRH1 | GYTFTTFA |
| | | | using IMGT | |
| 55 | CL-57945 | CDRH2(IMGT) | Amino acid sequence of CDRH2 | INPGNVKT |
| | | | using IMGT | |
| 56 | CL-57945 | CDRH3(IMGT) | Amino acid sequence of CDRH3 | ARRQLWLPFDY |
| | | | using IMGT | |
| 57 | CL-57945 | CDRH1 | Amino acid sequence of CDRH1 | TFAIH |
| | | (KABAT) | using Kabat | |
| 58 | CL-57945 | CDRH2 | Amino acid sequence of CDRH2 | WINPGNVKTDYSQKFQG |
| | | (KABAT) | using Kabat | |
| 59 | CL-57945 | CDRH3 | Amino acid sequence of CDRH3 | RQLWLPFDY |
| | | (KABAT) | using Kabat | |
| 60 | CL-57945 | Heavy chain | Amino acid sequence of V_{H} | QVHLVQSGAEVKNPGASVKVSCKASGYTFTTFAIHWLRQAPGQRLEWMGWINPIGNV |
| | | variable | | KTDYSQKFQGRVTISRDTSATTAYMELSSLRSEDTAVYYCARRQLWLPFDYWGQGTLVT |
| | | region | | VSS |
| 61 | CL-57945 | Heavy chain | Nucleotide sequence of V_{H} | CAGGTCCACCTTGTTCAGTCAGGGGCAGAGGTGAAGAACCCTGGGGCCTCAGTGAA |
| | | variable | | GGTCTCCTGCAAGGCTTCTGGATACACCTTCACTACCTTTGCTATTCATTGGTTGCGC |
| | | region | | CAGGCCCCCGGACAGAGGCTTGAGTGGATGGGATGGATCAACCCTGGCAATGTTAA |
| | | | | GACAGATTATTCGCAGAAGTTCCAGGGCAGAGTCACCATTAGCAGGGACACATCCG |
| | | | | CGACCACTGCCTACATGGAGCTGAGCAGCCTGAGATCTGAAGACACGGCTGTTTATT |
| | | | | ACTGTGCGAGAAGACAATTATGGTTACCCTTTGACTACTGGGGCCAGGGAACCCTG |
| | | | | GTCACCGTCTCCTCAG |
| 62 | CL-57945 | Full heavy | Amino acid sequence heavy | QVHLVQSGAEVKNPGASVKVSCKASGYTFTTFAIHWLRQAPGQRLEWMGWINPGNV |
| | | chain | chain | KTDYSQKFQGRVTISRDTSATTAYMELSSLRSEDTAVYYCARRQLWLPFDYWGQGLTVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 63 | CL-57945 | CDRL1(IMGT) | Amino acid sequence of CDRL1 | QSISNNF |
| | | | using IMGT | |
| — | CL-57945 | CDRL2(IMGT) | Amino acid sequence of CDRL2 | GAS |
| | | | using IMGT | |
| 65 | CL-57945 | CDRL3(IMGT) | Amino acid sequence of CDRL3 | QHYGGSPWT |
| | | | using IMGT | |
| 66 | CL-57945 | CDRL1 | Amino acid sequence of CDRL1 | RASQSISNNFLA |
| | | (KABAT) | using KABAT | |
| 67 | CL-57945 | CDRL2 | Amino acid sequence of CDRL2 | GASSRAT |
| | | (KABAT) | using KABAT | |
| 68 | CL-57945 | CDRL3 | Amino acid sequence of CDRL3 | QHYGGSPWT |
| | | (KABAT) | using KABAT | |
| 69 | CL-57945 | Light chain | Amino acid sequence of V_{L} | EIVLTQSPGTLSLSPGEGATLSCRASQSISNNFLAWYQQKPGQAPRLLIFGASSRATAIPD |
| | | variable | | RFVGSGSGTDFTLTITGLEPEDFAVYHCQHYGGSPWTFGQGTKVEIK |
| | | region | | |
| 70 | CL-57945 | Light chain | Amino acid sequence of V_{L} | GAAATTGTGTTGACGCAGTCTCCAGGCACCCTGTCTTTGTCTCCAGGGGAGGGAGCC |
| | | variable | | ACCCTCTCCTGCAGGGCCAGTCAGAGTATTAGCAACAACTTCTTAGCCTGGTACCAA |
| | | region | | CAGAAACCTGGCCAGGCTCCCAGACTCCTCATCTTTGGTGCATCCAGCAGGGCCACT |
| | | | | GCCATCCCAGACAGGTTCGTTGGCAGTGGGTCTGGGACAGACTTCACTCTCACCATC |
| | | | | ACCGGACTGGAGCCTGAAGATTTTGCAGTGTATCACTGTCAACACTATGGTGGTTCA |
| | | | | CCTTGGACGTTCGGCCAAGGGACCAAGGTGGAAATCAAA |
| 71 | CL-57945 | Full light | Amino acid sequence light chain | EIVLTQSPGTLSLSPGEGATLSCRASQSISNNFLAWYQQKPGQAPRLLIFGASSRATAIPD |
| | | chain | | RFVGSGSGTDFTLTITGLEPEDFAVYHCQHYGGSPWTFGQGTKVEIKRTVAAPSVFIFPP |
| | | sequence | | SDEQLKSGTASVVCLLNNFYPREAKVQWKVDNALQSGNSQESVTEQDSKDSTYSLSSTL |
| | | | | TLSKADYEKHKVYACEVTHQGLSSPVTKSFNRGEC |
| 72 | CL-58102 | CDRH1(IMGT) | Amino acid sequence of CDRH1 | GYSFTNYA |
| | | | using IMGT | |
| 73 | CL-58102 | CDRH2(IMGT) | Amino acid sequence of CDRH2 | IHAGNGKT |
| | | | using IMGT | |
| 74 | CL-58102 | CDRH3(IMGT) | Amino acid sequence of CDRH3 | ARRQLWLPFDY |
| | | | using IMGT | |
| 75 | CL-58102 | CDRH1 | Amino acid sequence of CDRH1 | NYALH |
| | | (KABAT) | using Kabat | |
| 76 | CL-58102 | CDRH2 | Amino acid sequence of CDRH2 | WIHAGNGKTEYAQKFQD |
| | | (KABAT) | using Kabat | |
| 77 | CL-58102 | CDRH3 | Amino acid sequence of CDRH3 | RQLWLPFDY |
| | | (KABAT) | using Kabat | |
| 78 | CL-58102 | Heavy chain | Amino acid sequence of V_{H} | QVQLVQSGAEVKKPGASVKVSCKASGYSFTNYALHWVRQAPGQRLEWMGWIHAGN |
| | | variable | | GKTEYAQKFQDRVTISRDISAITVYMELSSLRSEDTAVYYCARRQLWLPFDYWGQGTLVT |
| | | region | | VSS |
| 79 | CL-58102 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AAGTTTCCTGTAAGGCTTCTGGGTACAGTTTCACTAACTATGCTTTACATTGGGTGCG |
| | | region | | CCAGGCCCCCGGACAAAGACTTGAGTGGATGGGATGGATCCACGCTGGTAATGGTA |
| | | | | AGACAGAATATGCACAGAAGTTCCAGGACAGAGTCACCATTAGTAGGGACATATCC |
| | | | | GCGATCACAGTTTACATGGAACTGAGCAGCCTGAGATCTGAAGACACGGCTGTTTAT |
| | | | | TATTGTGCGAGAAGACAGTTATGGTTACCCTTTGACTACTGGGGCCAGGGAACCCTG |
| | | | | GTCACCGTCTCCTCA |
| 80 | CL-58102 | Full heavy | Amino acid sequence heavy | QVQLVQSGAEVKKPGASVKVSCKASGYSFTNYALHWVRQAPGQRLEWMGWIHAGN |
| | | chain | chain | GKTEYAQKFQDRVTISRDISAITVYMELSSLRSEDTAVYYCARRQLWLPFDYWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 81 | CL-58102 | CDRL1(IMGT) | Amino acid sequence of CDRL1 | QIIINRQ |
| | | | using IMGT | |
| — | CL-58102 | CDRL2(IMGT) | Amino acid sequence of CDRL2 | GAS |
| | | | using IMGT | |
| 83 | CL-58102 | CDRL3(IMGT) | Amino acid sequence of CDRL3 | QHYGGSPWT |
| | | | using IMGT | |
| 84 | CL-58102 | CDRL1 | Amino acid sequence of CDRL1 | RAGQIIINRQLA |
| | | (KABAT) | using KABAT | |
| 85 | CL-58102 | CDRL2 | Amino acid sequence of CDRL2 | GASNRVT |
| | | (KABAT) | using KABAT | |
| 86 | CL-58102 | CDRL3 | Amino acid sequence of CDRL3 | QHYGGSPWT |
| | | (KABAT) | using KABAT | |
| 87 | CL-58102 | Light chain | Amino acid sequence of V_{L} | EIVLTQSPDTLSLSPGETASFSCRAGQIIINRQLAWYQRRPGQAPRLLIYGASNRVTGIPD |
| | | variable | | RFSGSGSGTDFTLTINRLEPEDFAVYYCQHYGGSPWTFGQGTKVEIK |
| | | region | | |
| 88 | CL-58102 | Light chain | Nucleic acid sequence of V_{L} | GAAATTGTGTTGACGCAGTCTCCAGACACCCTCTCTTTGTCTCCAGGGGAAACAGCC |
| | | variable | | AGTTTCTCCTGCAGGGCCGGTCAAATTATTATCAACAGACAGTTAGCCTGGTACCAG |
| | | region | | CGGAGACCTGGCCAGGCTCCCCGGCTCCTCATCTATGGCGCGTCCAATAGGGTCACT |
| | | | | GGCATCCCAGACAGGTTCAGTGGCAGTGGGTCTGGGACAGACTTCACTCTCACGAT |
| | | | | CAATAGACTGGAGCCTGAAGATTTTGCAGTGTATTACTGTCAGCACTATGGTGCTC |
| | | | | ACCTTGGACGTTCGGCCAAGGGACCAAGGTGGAAATCAAA |
| 89 | CL-58102 | Full light | Amino acid sequence light chain | EIVLTQSPDTLSLSPGETASFSCRAGQIIINRQLAWYQRRPGQAPRLLIYGASNRVTGIPD |
| | | chain | | RFSGSGSGTDFTLTINRLEPEDFAVYYCQHYGGSPWTFGQGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 90 | CL-58252 | CDRH1(IMGT) | Amino acid sequence of CDRH1 | GYTFTSHA |
| | | | using IMGT | |
| 91 | CL-58252 | CDRH2(IMGT) | Amino acid sequence of CDRH2 | INAANGKT |
| | | | using IMGT | |
| 92 | CL-58252 | CDRH3(IMGT) | Amino acid sequence of CDRH3 | ARRPYGGPFDY |
| | | | using IMGT | |
| 93 | CL-58252 | CDRH1 | Amino acid sequence of CDRH1 | SHAMH |
| | | (KABAT) | using Kabat | |
| 94 | CL-58252 | CDRH2 | Amino acid sequence of CDRH2 | WINAANGKTDYSQNFQG |
| | | (KABAT) | using Kabat | |
| 95 | CL-58252 | CDRH3 | Amino acid sequence of CDRH3 | RPYGGPFDY |
| | | (KABAT) | using Kabat | |
| 96 | CL-58252 | Heavy chain | Amino acid sequence of V_{H} | QVQFVQSGAEVKKPGASVKVSCKASGYTFTSHAMHWVRQAPGQRLEWMGWINAAN |
| | | variable | | GKTDYSQNFQGRVTITRDTYANTVYMELSSLRSEDTAVYYCARRPYGGPFDYWGQGTL |
| | | region | | VTVSS |
| 97 | CL-58252 | Heavy chain | Nucleotide sequence of V_{H} | CAGGTCCAGTTTGTGCAGTCTGGGGCTGAGGTGAAGAAGCCTGGGGCCTCAGTGAA |
| | | variable | | GGTTTCCTGTAAGGCTTCTGGATACACCTTCACTAGCCATGCTATGCATTGGGTGCG |
| | | region | | CCAGGCCCCCGGACAAAGGCTTGAGTGGATGGGATGGATCAACGCTGCCAATGGTA |
| | | | | AAACAGATTATTCACAGAACTTCCAGGGCAGAGTCACCATTACCAGGGACACATACG |
| | | | | CGAACACAGTCTACATGGAACTGAGCAGCCTGAGATCTGAAGACACGGCTGTGTAT |
| | | | | TACTGTGCGAGACGCCCTTACGGTGGTCCCTTTGACTACTGGGGCCAGGGAACCTG |
| | | | | GTCACCGTCTCCTCA |
| 98 | CL-58252 | Full heavy | Amino acid sequence heavy | QVQFVQSGAEVKKPGASVKVSCKASGYTFTSHAMHWVRQAPGQRLEWMGWINAAN |
| | | chain | chain | GKTDYSQNFQGRVTITRDTYANTVYMELSSLRSEDTAVYYCARRPYGGPFDYWGQGTL |
| | | sequence | | VTVSSASTKGPSVFPLAPCSRSTSESTAALGCLVKDYFPEPVTVSWNSGALTSGVHTFPA |
| | | | | VLQSSGLYSLSSVVTVPSSSLGTKTYTCNVDHKPSNTKVDKRVESKYGPPCPPCPAPEFEG |
| | | | | GPSVFLFPPKPKDTLMISRTPEVTCVVVDVSQEDPEVQFNWYVDGVEVHNAKTKFPREE |
| | | | | QFNSTYRVVSVLTVLHQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLDSDGSFFLYSRLTVD |
| | | | | KSRWQEGNVFSCSVMHEALHNHYTQKSLSLSLGK |
| 99 | CL-58252 | CDRL1(IMGT) | Amino acid sequence of CDRL1 | QSISIW |
| | | | using IMGT | |
| 100 | CL-58252 | CDRL2(IMGT) | Amino acid sequence of CDRL2 | KAS |
| | | | using IMGT | |
| 101 | CL-58252 | CDRL3(IMGT) | Amino acid sequence of CDRL3 | QQYNLYPWT |
| | | | using IMGT | |
| 102 | CL-58252 | CDRL1 | Amino acid sequence of CDRL1 | RASQSISIWLA |
| | | (KABAT) | using KABAT | |
| 103 | CL-58252 | CDRL2 | Amino acid sequence of CDRL2 | KASSLES |
| | | (KABAT) | using KABAT | |
| 104 | CL-58252 | CDRL3 | Amino acid sequence of CDRL3 | QQYNLYPWT |
| | | (KABAT) | using KABAT | |
| 105 | CL-58252 | Light chain | Amino acid sequence of V_{L} | DIQMTQSPSTLSASVGDRVTITCRASQSISIWLAWYQQKPGKAPKLLIYKASSLESGVPSR |
| | | variable | | FSGSGSGTEFTLTISSLQPDDFATYYCQQYNLYPWTFGQGTKVEIK |
| | | region | | |
| 106 | CL-58252 | Light chain | Nucleic acid sequence of V_{L} | GACATCCAGATGACCCAGTCTCCTTCCACCCTGTCTGCATCTGTAGGAGACAGAGTC |
| | | variable | | ACCATCACTTGCCGGGCCAGTCAGAGTATTAGTATCTGGTTGGCCTGGTATCAGCAG |
| | | region | | AAACCAGGGAAAGCCCCTAAGCTCCTGATCTATAAGGCGTCTAGTTTAGAAAGTGG |
| | | | | GGTCCCATCAAGGTTCAGCGGCAGTGGATCTGGGACAGAATTCACTCTCACCATCAG |
| | | | | CAGCCTGCAGCCTGATGATTTTGCAACTTATTACTGTCAACAGTATAATCTTTATCCG |
| | | | | TGGACGTTCGGCCAAGGGACCAAGGTGGAAATCAAA |
| 107 | CL-58252 | Full light | Amino acid sequence light chain | DIQMTQSPSTLSASVGDRVTITCRASQSISIWLAWYQQKPGKAPKLLIYKASSLESGVPSR |
| | | chain | | FSGSGSGTEFTLTISSLQPDDFATYYCQQYNLYPWTFGQGTKVEIKRTVAAPSVFIFPPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 108 | CL-58838 | CDRH1(IMGT) | Amino acid sequence of CDRH1 | GFTFSDYY |
| | | | using IMGT | |
| 109 | CL-58838 | CDRH2(IMGT) | Amino acid sequence of CDRH2 | ISISGSTI |
| | | | using IMGT | |
| 110 | CL-58838 | CDRH3(IMGT) | Amino acid sequence of CDRH3 | ARRSSGWYDY |
| | | | using IMGT | |
| 111 | CL-58838 | CDRH1 | Amino acid sequence of CDRH1 | DYYMS |
| | | (KABAT) | using Kabat | |
| 112 | CL-58838 | CDRH2 | Amino acid sequence of CDRH2 | YISISGSTIYYADSVKG |
| | | (KABAT) | using Kabat | |
| 113 | CL-58838 | CDRH3 | Amino acid sequence of CDRH3 | RSSGWYDY |
| | | (KABAT) | using Kabat | |
| 114 | CL-58838 | Heavy chain | Amino acid sequence of V_{H} | QVQLVESGGGLVKPGGSLRLSCAASGFTFSDYYMSWIRQAPGKGLEWVSYISISGSTIYY |
| | | variable | | ADSVKGRFTISRDNAMDSLYLQMNSLRAEDTAVYYCARRSSGWYDYWGQGTLVTVSS |
| | | region | | |
| 115 | CL-58838 | Heavy chain | Nucleotide sequence of V_{H} | CAGGTGCAACTGGTGGAGTCTGGGGGAGGCTTGGTCAAGCCTGGAGGGTCCCTGA |
| | | variable | | GACTCTCCTGTGCAGCCTCTGGATTCACCTTCAGTGACTACTACATGAGTTGGATCCG |
| | | region | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCA |
| 116 | CL-58838 | Full heavy | Amino acid sequence heavy | QVQLVESGGGLVKPGGSLRLSCAASGFTFSDYYMSWIRQAPGKGLEWVSYISISGSTIYY |
| | | chain | chain | ADSVKGRFTISRDNAMDSLYLQMNSLRAEDTAVYYCARRSSGWYDYWGQGTLVTVSS |
| | | sequence | | ASTKGPSVFPLAPCSRSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPPCPPCPAPEFEGGPSVF |
| | | | | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 117 | CL-58838 | CDRL1(IMGT) | Amino acid sequence of CDRL1 | QRVVYRY |
| | | | using IMGT | |
| — | CL-58838 | CDRL2(IMGT) | Amino acid sequence of CDRL2 | GAF |
| | | | using IMGT | |
| 119 | CL-58838 | CDRL3(IMGT) | Amino acid sequence of CDRL3 | HQYGSSPPT |
| | | | using IMGT | |
| 120 | CL-58838 | CDRL1 | Amino acid sequence of CDRL1 | WASQRVVYRYLA |
| | | (KABAT) | using KABAT | |
| 121 | CL-58838 | CDRL2 | Amino acid sequence of CDRL2 | GAFNRAT |
| | | (KABAT) | using KABAT | |
| 122 | CL-58838 | CDRL3 | Amino acid sequence of CDRL3 | HQYGSSPPT |
| | | (KABAT) | using KABAT | |
| 123 | CL-58838 | Light chain | Amino acid sequence of V_{L} | EIVLTQSPGTLSLSPGERATLSCWASQRVVYRYLAWYQRKPGQAPRLLIYGAFNRATGIP |
| | | variable | | DRFSGSGSGTDFSLTISRLEPEDFAVYYCHQYGSSPPTFGQGTKVEIK |
| | | region | | |
| 124 | CL-58838 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCTTGTTGGGCCAGTCAGAGGGTTGTTTACAGATACTTAGCCTGGTACCAG |
| | | region | | CGGAAACCTGGCCAGGCTCCCAGACTTCTCATTTATGGTGCATTCAACAGGGCCACT |
| | | | | GGCATCCCAGACAGGTTCAGTGGCAGTGGGTCTGGGACAGACTTCAGTCTCACTAT |
| | | | | CAGTAGACTGGAGCCTGAGGATTTTGCAGTTTATTACTGTCACCAATATGGTAGTTC |
| | | | | ACCACCGACGTTCGGCCAAGGGACCAAGGTGGAAATCAAA |
| 125 | CL-58838 | Full light | Amino acid sequence light chain | EIVLTQSPGTLSLSPGERATLSCWASQRVVYRYLAWYQRKPGQAPRLLIYGAFNRATGIP |
| | | chain | | DRFSGSGSGTDFSLTISRLEPEDFAVYYCHQYGSSPPTFGQGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 126 | CL-58851 | CDRH1(IMGT) | Amino acid sequence of CDRH1 | GFTFITYA |
| | | | using IMGT | |
| 127 | CL-58851 | CDRH2(IMGT) | Amino acid sequence of CDRH2 | INVGNGNR |
| | | | using IMGT | |
| 128 | CL-58851 | CDRH3(IMGT) | Amino acid sequence of CDRH3 | ARRPLWGPFDY |
| | | | using IMGT | |
| 129 | CL-58851 | CDRH1 | Amino acid sequence of CDRH1 | TYAFH |
| | | (KABAT) | using Kabat | |
| 130 | CL-58851 | CDRH2 | Amino acid sequence of CDRH2 | WINVGNGNREYSQKFQD |
| | | (KABAT) | using Kabat | |
| 131 | CL-58851 | CDRH3 | Amino acid sequence of CDRH3 | RPLWGPFDY |
| | | (KABAT) | using Kabat | |
| 132 | CL-58851 | Heavy chain | Amino acid sequence of V_{H} | QVQLVQSGAEVKKPGASVKVSCKATGFTFITYAFHWVRQAPGQRFEWMGWINVGNG |
| | | variable | | NREYSQKFQDRVTITRDTSATTVYMELNSLKSEDTAMYFCARRPLWGPFDYWGQGTLV |
| | | region | | TVSS |
| 133 | CL-58851 | Heavy chain | Nucleotide sequence of V_{H} | CAGGTCCAACTTGTGCAGTCTGGGGCTGAGGTGAAGAAGCCTGGGGCCTCAGTGAA |
| | | variable | | GGTTTCCTGCAAGGCTACTGGATTCACCTTCATTACCTATGCTTTCCATTGGGTGCGC |
| | | region | | CAGGCCCCCGGACAAAGGTTTGAGTGGATGGGATGGATCAACGTTGGCAATGGTAA |
| | | | | CAGAGAATATTCACAGAAGTTCCAGGACAGAGTCACCATTACCAGGGACACATCCG |
| | | | | CGACCACAGTCTACATGGAACTGAACAGCCTGAAATCTGAAGACACGGCTATGTATT |
| | | | | TCTGTGCGAGACGCCCCCTCTGGGGTCCCTTTGACTACTGGGGCCAGGGAACCCTG |
| | | | | GTCACCGTCTCCTCA |
| 134 | CL-58851 | Full heavy | Amino acid sequence heavy | QVQLVQSGAEVKKPGASVKVSCKATGFTFITYAFHWVRQAPGQRFEWMGWINVGNG |
| | | chain | chain | NREYSQKFQDRVTITRDTSATTVYMELNSLKSEDTAMYFCARRPLWGPFDYWGQGTLV |
| | | sequence | | TVSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 135 | CL-58851 | CDRL1(IMGT) | Amino acid sequence of CDRL1 | QIFSNTF |
| | | | using IMGT | |
| — | CL-58851 | CDRL2(IMGT) | Amino acid sequence of CDRL2 | GAS |
| | | | using IMGT | |
| 137 | CL-58851 | CDRL3(IMGT) | Amino acid sequence of CDRL3 | QHYGGSPWT |
| | | | using IMGT | |
| 138 | CL-58851 | CDRL1 | Amino acid sequence of CDRL1 | RASQIFSNTFLA |
| | | (KABAT) | using KABAT | |
| 139 | CL-58851 | CDRL2 | Amino acid sequence of CDRL2 | GASKRAT |
| | | (KABAT) | using KABAT | |
| 140 | CL-58851 | CDRL3 | Amino acid sequence of CDRL3 | QHYGGSPWT |
| | | (KABAT) | using KABAT | |
| 141 | CL-58851 | Light chain | Amino acid sequence of V_{L} | EIVLTQSPGTLSLSPGERATLSCRASQIFSNTFLAWYQQKPGQAPRLLVYGASKRATAIPD |
| | | variable | | RFSGSGSGTDFILTINRLEPEDFAVYYCQHYGGSPWTFGRGTKVEIK |
| | | region | | |
| 142 | CL-58851 | Light chain | Nucleic acid sequence of V_{L} | GAAATTGTTTTGACACAGTCTCCAGGCACCCTGTCTTTGTCTCCAGGGGAAAGAGCC |
| | | variable | | ACCCTCTCCTGCAGGGCCAGTCAGATTTTTAGCAACACCTTCTTAGCCTGGTACCAGC |
| | | region | | AGAAACCTGGCCAGGCTCCCAGGCTCCTCGTGTATGGTGCATCCAAGAGGGCCACT |
| | | | | GCCATCCCAGACAGGTTTAGTGGCAGTGGGTCTGGGACAGACTTCATTCTCACCATC |
| | | | | AACAGACTGGAGCCTGAAGATTTTGCAGTATATTACTGTCAACACTATGGTGGGTCA |
| | | | | CCGTGGACGTTCGGCCGAGGGACCAAGGTGGAAATCAAA |
| 143 | CL-58851 | Full light | Amino acid sequence light chain | EIVLTQSPGTLSLSPGERATLSCRASQIFSNTFLAWYQQKPGQAPRLLVYGASKRATAIPD |
| | | chain | | RFSGSGSGTDFILTINRLEPEDFAVYYCQHYGGSPWTFGRGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 144 | CL-75183 | CDRH1(IMGT) | Amino acid sequence of CDRH1 | GYTFTSHA |
| | | | using IMGT | |
| 145 | CL-75183 | CDRH2(IMGT) | Amino acid sequence of CDRH2 | IHAGNGNS |
| | | | using IMGT | |
| 146 | CL-75183 | CDRH3(IMGT) | Amino acid sequence of CDRH3 | ARRAIMAPFDL |
| | | | using IMGT | |
| 147 | CL-75183 | CDRH1 | Amino acid sequence of CDRH1 | SHAIH |
| | | (KABAT) | using Kabat | |
| 148 | CL-75183 | CDRH2 | Amino acid sequence of CDRH2 | WIHAGNGNSKQSQNFQD |
| | | (KABAT) | using Kabat | |
| 149 | CL-75183 | CDRH3 | Amino acid sequence of CDRH3 | RAIMAPFDL |
| | | (KABAT) | using Kabat | |
| 150 | CL-75183 | Heavy chain | Amino acid sequence of V_{H} | QVQLVQSGAEVKKPGASVKVSCKASGYTFTSHAIHWVRQAPGQRLEWMGWIHAGNG |
| | | variable | | NSKQSQNFQDRVTITRDTSASAAYMELSSLRSEDTAVYYCARRAIMAPFDLWGQGTLV |
| | | region | | TVSS |
| 151 | CL-75183 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AGGTTTCCTGCAAGGCTTCTGGATACACCTTCACTAGCCATGCTATACATTGGGTGC |
| | | region | | GCCAGGCCCCCGGACAACGGCTTGAGTGGATGGGATGGATCCACGCTGGCAATGG |
| | | | | TAACTCAAAACAGTCACAGAACTTCCAGGACAGAGTCACCATTACCAGGGACACATC |
| | | | | CGCGAGCGCAGCCTACATGGAGCTGAGCAGCCTGAGATCTGAAGACACGGCTGTAT |
| | | | | ATTACTGTGCGAGACGGGCCATAATGGCCCCGTTTGACCTCTGGGGCCAGGGAACC |
| | | | | CTGGTCACCGTCTCCTCA |
| 152 | CL-75183 | Full heavy | Amino acid sequence heavy | QVQLVQSGAEVKKPGASVKVSCKASGYTFTSHAIHWVRQAPGQRLEWMGWIHAGNG |
| | | chain | chain | NSKQSQNFQDRVTITRDTSASAAYMELSSLRSEDTAVYYCARRAIMAPFDLWGQGTLV |
| | | sequence | | TVSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 153 | CL-75183 | CDRL1(IMGT) | Amino acid sequence of CDRL1 | QSINNW |
| | | | using IMGT | |
| — | CL-75183 | CDRL2(IMGT) | Amino acid sequence of CDRL2 | KAS |
| | | | using IMGT | |
| 155 | CL-75183 | CDRL3(IMGT) | Amino acid sequence of CDRL3 | QQYYSSWT |
| | | | using IMGT | |
| 156 | CL-75183 | CDRL1 | Amino acid sequence of CDRL1 | RASQSINNWLA |
| | | (KABAT) | using KABAT | |
| 157 | CL-75183 | CDRL2 | Amino acid sequence of CDRL2 | KASSLES |
| | | (KABAT) | using KABAT | |
| 158 | CL-75183 | CDRL3 | Amino acid sequence of CDRL3 | QQYYSSWT |
| | | (KABAT) | using KABAT | |
| 159 | CL-75183 | Light chain | Amino acid sequence of V_{L} | DIQMTQSPSTLSASVGDRVTITCRASQSINNWLAWYQQKPGKAPNLLIYKASSLESGVP |
| | | variable | | SRFSGSGSGTEFTLTINSLQPDDFATYYCQQYYSSWTFGQGTKVEIK |
| | | region | | |
| 160 | CL-75183 | Light chain | Nucleic acid sequence of V_{L} | GACATCCAGATGACCCAGTCTCCTTCCACCCTGTCTGCATCTGTAGGAGACAGAGTC |
| | | variable | | ACCATCACTTGCCGGGCCAGTCAGAGTATTAATAACTGGTTGGCCTGGTATCAGCAG |
| | | region | | AAACCAGGGAAAGCCCCTAACCTCCTGATCTATAAGGCGTCTAGTTTAGAAAGTGG |
| | | | | GGTCCCATCAAGGTTCAGCGGCAGTGGATCTGGGACAGAATTCACTCTCACCATCAA |
| | | | | CAGCCTGCAGCCTGATGACTTTGCAACTTATTACTGCCAACAGTATTATAGTTCTTGG |
| | | | | ACGTTCGGCCAAGGGACCAAGGTGGAAATCAAA |
| 161 | CL-75183 | Full light | Amino acid sequence light chain | DIQMTQSPSTLSASVGDRVTITCRASQSINNWLAWYQQKPGKAPNLLIYKASSLESGVP |
| | | chain | | SRFSGSGSGTEFTLTINSLQPDDFATYYCQQYYSSWTFGQGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 162 | CL-75500 | CDRH1(IMGT) | Amino acid sequence of CDRH1 | GYTFTSYA |
| | | | using IMGT | |
| 163 | CL-75500 | CDRH2(IMGT) | Amino acid sequence of CDRH2 | INAGNGNT |
| | | | using IMGT | |
| 164 | CL-75500 | CDRH3(IMGT) | Amino acid sequence of CDRH3 | ARRGFGEPFDY |
| | | | using IMGT | |
| 165 | CL-75500 | CDRH1 | Amino acid sequence of CDRH1 | SYAVH |
| | | (KABAT) | using Kabat | |
| 166 | CL-75500 | CDRH2 | Amino acid sequence of CDRH2 | WINAGNGNTKFSQKFQG |
| | | (KABAT) | using Kabat | |
| 167 | CL-75500 | CDRH3 | Amino acid sequence of CDRH3 | RGFGEPFDY |
| | | (KABAT) | using Kabat | |
| 168 | CL-75500 | Heavy chain | Amino acid sequence of V_{H} | QVQLVQSGAEVKKPGASVKVSCKASGYTFTSYAVHWVRQAPGQRLEWMGWINAGN |
| | | variable | | GNTKFSQKFQGRITITRDTSASTTYMELNSLRSEDTAVYYCARRGFGEPFDYWGQGLTV |
| | | region | | TVSS |
| 169 | CL-75500 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AGGTTTCCTGCAAGGCTTCTGGATACACCTTCACTAGCTATGCTGTCCATTGGGTGC |
| | | region | | GCCAGGCCCCCGGACAAAGGCTTGAGTGGATGGGATGGATCAACGCTGGCAATGG |
| | | | | TAACACAAAATTTTCACAGAAGTTCCAGGGCAGAATCACCATTACCAGGGACACATC |
| | | | | CGCGAGCACAACCTACATGGAGTTGAATAGTCTGAGATCTGAAGACACGGCTGTGT |
| | | | | ATTATTGTGCGAGAAGGGGGTTCGGGGAGCCATTTGACTACTGGGGCCAGGGAAC |
| | | | | CCTGGTCACCGTCTCCTCA |
| 170 | CL-75500 | Full heavy | Amino acid sequence heavy | QVQLVQSGAEVKKPGASVKVSCKASGYTFTSYAVHWVRQAPGQRLEWMGWINAGN |
| | | chain | chain | GNTKFSQKFQGRITITRDTSASTTYMELNSLRSEDTAVYYCARRGFGEPFDYWGQGTLV |
| | | sequence | | TVSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 171 | CL-75500 | CDRL1(IMGT) | Amino acid sequence of CDRL1 | QSISNN |
| | | | using IMGT | |
| — | CL-75500 | CDRL2(IMGT) | Amino acid sequence of CDRL2 | GTS |
| | | | using IMGT | |
| 173 | CL-75500 | CDRL3(IMGT) | Amino acid sequence of CDRL3 | QQYNIWPFT |
| | | | using IMGT | |
| 174 | CL-75500 | CDRL1 | Amino acid sequence of CDRL1 | RASQSISNNLA |
| | | (KABAT) | using KABAT | |
| 175 | CL-75500 | CDRL2 | Amino acid sequence of CDRL2 | GTSTRAT |
| | | (KABAT) | using KABAT | |
| 176 | CL-75500 | CDRL3 | Amino acid sequence of CDRL3 | QQYNIWPFT |
| | | (KABAT) | using KABAT | |
| 177 | CL-75500 | Light chain | Amino acid sequence of V_{L} | EIVMTQSPATLSVSPGERATLSCRASQSISNNLAWYQQKPGQAPRLLIYGTSTRATGIPA |
| | | variable | | RFSGSGSGTEFTLTISTLQSEDFAVYYCQQYNIWPFTFGPGTKVDIK |
| | | region | | |
| 178 | CL-75500 | Light chain | Nucleic acid sequence of V_{L} | GAAATAGTGATGACGCAGTCTCCAGCCACCCTGTCTGTGTCTCCAGGGGAAAGAGC |
| | | variable | | CACCCTCTCCTGCAGGGCCAGTCAGAGTATTAGTAACAACTTAGCCTGGTACCACA |
| | | region | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAA |
| 179 | CL-75500 | Full light | Amino acid sequence light chain | EIVMTQSPATLSVSPGERATLSCRASQSISNNLAWYQQKPGQAPRLLIYGTSTRATGIPA |
| | | chain | | RFSGSGSGTEFTLTISTLQSEDFAVYYCQQYNIWPFTFGPGTKVDIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 180 | CL-75506 | CDRH1(IMGT) | Amino acid sequence of CDRH1 | GYTFTSYA |
| | | | using IMGT | |
| 181 | CL-75506 | CDRH2(IMGT) | Amino acid sequence of CDRH2 | INPGNGNT |
| | | | using IMGT | |
| 182 | CL-75506 | CDRH3(IMGT) | Amino acid sequence of CDRH3 | ARRGFGEPFDY |
| | | | using IMGT | |
| 183 | CL-75506 | CDRH1 | Amino acid sequence of CDRH1 | SYAIH |
| | | (KABAT) | using Kabat | |
| 184 | CL-75506 | CDRH2 | Amino acid sequence of CDRH2 | WINPGNGNTKFSQKFQG |
| | | (KABAT) | using Kabat | |
| 185 | CL-75506 | CDRH3 | Amino acid sequence of CDRH3 | RGFGEPFDY |
| | | (KABAT) | using Kabat | |
| 186 | CL-75506 | Heavy chain | Amino acid sequence of V_{H} | QVQLVQSGAEVKKPGASVKVSCKASGYTFTSYAIHWVRQAPGQRLEWMGWINPGNG |
| | | variable | | NTKFSQKFQGRITITRDTSASTTYMELNSLRSEDTAVYYCARRGFGEPFDYWGQGTLVT |
| | | region | | VSS |
| 187 | CL-75506 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AGGTTTCCTGCAAGGCTTCTGGATACACCTTCACTAGCTATGCTATCCATTGGGTGCG |
| | | region | | CCAGGCCCCCGGACAAAGGCTTGAGTGGATGGGATGGATCAACCCTGGCAATGGTA |
| | | | | ACACAAAATTTTCACAGAAGTTCCAGGGCAGAATCACCATTACCAGGGACACATCCG |
| | | | | CGAGCACAACCTACATGGAGCTGAACAGCCTGAGATCTGAAGACACGGCTGTGTAT |
| | | | | TACTGTGCGAGAAGGGGGTTCGGGGAGCCATTTGACTACTGGGGCCAGGGAACCC |
| | | | | TGGTCACCGTCTCCTCA |
| 188 | CL-75506 | Full heavy | Amino acid sequence heavy | QVQLVQSGAEVKKPGASVKVSCKASGYTFTSYAIHWVRQAPGQRLEWMGWINPGNG |
| | | chain | chain | NTKFSQKFQGRITITRDTSASTTYMELNSLRSEDTAVYYCARRGFGEPFDYWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 189 | CL-75506 | CDRL1(IMGT) | Amino acid sequence of CDRL1 | QSISSN |
| | | | using IMGT | |
| — | CL-75506 | CDRL2(IMGT) | Amino acid sequence of CDRL2 | GTS |
| | | | using IMGT | |
| 191 | CL-75506 | CDRL3(IMGT) | Amino acid sequence of CDRL3 | QQYNIWPFT |
| | | | using IMGT | |
| 192 | CL-75506 | CDRL1 | Amino acid sequence of CDRL1 | RASQSISSNLA |
| | | (KABAT) | using KABAT | |
| 193 | CL-75506 | CDRL2 | Amino acid sequence of CDRL2 | GTSTRAT |
| | | (KABAT) | using KABAT | |
| 194 | CL-75506 | CDRL3 | Amino acid sequence of CDRL3 | QQYNIWPFT |
| | | (KABAT) | using KABAT | |
| 195 | CL-75506 | Light chain | Amino acid sequence of V_{L} | EIVMTQSPATLSVSPGERATLSCRASQSISSNLAWYQQNPGQAPRLLIYGTSTRATGIPA |
| | | variable | | RFSGSGSGTEFTLTISTLQSEDFAVYYCQQYNIWPFTFGPGTKVDIK |
| | | region | | |
| 196 | CL-75506 | Light chain | Nucleic acid sequence of V_{L} | GAAATAGTGATGACGCAGTCTCCAGCCACCCTGTCTGTGTCTCCAGGGGAAAGAGC |
| | | variable | | CACCCTCTCCTGCAGGGCCAGTCAGAGTATTAGCAGCAACTTAGCCTGGTACCAACA |
| | | region | | GAACCCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAAC |
| 197 | CL-75506 | Full light | Amino acid sequence light chain | EIVMTQSPATLSVSPGERATLSCRASQSISSNLAWYQQNPGQAPRLLIYGTSTRATGIPA |
| | | chain | | RFSGSGSGTEFTLTISTLQSEDFAVYYCQQYNIWPFTFGPGTKVDIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 198 | CL-75520 | CDRH1(IMGT) | Amino acid sequence of CDRH1 | GYTFTSYA |
| | | | using IMGT | |
| 199 | CL-75520 | CDRH2(IMGT) | Amino acid sequence of CDRH2 | INAGNGYT |
| | | | using IMGT | |
| 200 | CL-75520 | CDRH3(IMGT) | Amino acid sequence of CDRH3 | ARDRITIIRPFDY |
| | | | using IMGT | |
| 201 | CL-75520 | CDRH1 | Amino acid sequence of CDRH1 | SYAMH |
| | | (KABAT) | using Kabat | |
| 202 | CL-75520 | CDRH2 | Amino acid sequence of CDRH2 | WINAGNGYTKYSQKFQD |
| | | (KABAT) | using Kabat | |
| 203 | CL-75520 | CDRH3 | Amino acid sequence of CDRH3 | DRITIIRPFDY |
| | | (KABAT) | using Kabat | |
| 204 | CL-75520 | Heavy chain | Amino acid sequence of V_{H} | QVQLVQSGAEVKKPGASVKVSCKASGYTFTSYAMHWVRQAPGHRLEWLGWINAGNG |
| | | variable | | YTKYSQKFQDRVAITRDTSASTAFMELSSLRSEDTAVYYCARDRITIIRPFDYWGQGTLVT |
| | | region | | VSS |
| 205 | CL-75520 | Heavy chain | Nucleotide sequence of V_{H} | CAGGTCCAACTTGTGCAGTCTGGGGCTGAGGTGAAGAAGCCTGGGGCCTCAGTGAA |
| | | variable | | GGTTTCCTGCAAGGCTTCTGGATACACCTTCACTAGCTATGCTATGCATTGGGTGCGT |
| | | region | | CAGGCCCCCGGACACAGGCTTGAGTGGTTGGGATGGATCAACGCTGGCAATGGTTA |
| | | | | CACAAAATATTCACAGAAATTCCAGGACAGAGTCGCCATTACCAGGGACACATCCGC |
| | | | | GAGCACAGCCTTCATGGAGCTGAGCAGCCTGAGATCTGAAGACACGGCTGTGTATT |
| | | | | ACTGTGCGAGAGATCGTATTACTATTATTCGGCCCTTTGACTACTGGGGCCAGGGAA |
| | | | | CCCTGGTCACCGTCTCCTCA |
| 206 | CL-75520 | Full heavy | Amino acid sequence heavy | QVQLVQSGAEVKKPGASVKVSCKASGYTFTSYAMHWVRQAPGHRLEWLGWINAGNG |
| | | chain | chain | YTKYSQKFQDRVAITRDTSASTAFMELSSLRSEDTAVYYCARDRITIIRPFDYWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 207 | CL-75520 | CDRL1(IMGT) | Amino acid sequence of CDRL1 | QSISSSY |
| | | | using IMGT | |
| — | CL-75520 | CDRL2(IMGT) | Amino acid sequence of CDRL2 | GAS |
| | | | using IMGT | |
| 209 | CL-75520 | CDRL3(IMGT) | Amino acid sequence of CDRL3 | QLYGSPFT |
| | | | using IMGT | |
| 210 | CL-75520 | CDRL1 | Amino acid sequence of CDRL1 | RASQSISSSYLA |
| | | (KABAT) | using KABAT | |
| 211 | CL-75520 | CDRL2 | Amino acid sequence of CDRL2 | GASSRAT |
| | | (KABAT) | using KABAT | |
| 212 | CL-75520 | CDRL3 | Amino acid sequence of CDRL3 | QLYGSPFT |
| | | (KABAT) | using KABAT | |
| 213 | CL-75520 | Light chain | Amino acid sequence of V_{L} | EIVLTQSPGTLSLSPGERATLSCRASQSISSSYLAWFQQKPGQAPRLLIYGASSRATGIPDR |
| | | variable | | FSGNGSGTDFTLTISRLEPEDFAVYYCQLYGSPFTFGPGTKMDIK |
| | | region | | |
| 214 | CL-75520 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCCTGCAGGGCCAGTCAGAGTATTAGCAGCAGCTACTTAGCCTGGTTCCAG |
| | | region | | CAAAAACCTGGCCAGGCTCCCAGGCTCCTCATCTATGGTGCATCCAGCAGGGCCACT |
| | | | | GGCATCCCAGACAGGTTCAGTGGCAATGGGTCTGGGACAGACTTCACTCTCACCATC |
| | | | | AGCAGACTGGAGCCTGAAGATTTTGCAGTGTATTACTGTCAGCTGTATGGTAGCCCA |
| | | | | TTCACTTTCGGCCCTGGGACCAAAATGGATATTAAAC |
| 215 | CL-75520 | Full light | Amino acid sequence light chain | EIVLTQSPGTLSLSPGERATLSCRASQSISSSYLAWFQQKPGQAPRLLIYGASSRATGIPDR |
| | | chain | | FSGNGSGTDFTLTISRLEPEDFAVYYCQLYGSPFTFGPGTKMDIKRTVAAPSVFIFPPSDE |
| | | sequence | | QLKSGTASVVCLLNNFYPREAKVQWKVDNALQSGNSQESVTEQDSKDSTYSLSSTLTLS |
| | | | | KADYEKHKVYACEVTHQGLSSPVTKSFNRGEC |
| 216 | CL-75539 | CDRH1(IMGT) | Amino acid sequence of CDRH1 | GYTFTSYA |
| | | | using IMGT | |
| 217 | CL-75539 | CDRH2(IMGT) | Amino acid sequence of CDRH2 | INVGNGKT |
| | | | using IMGT | |
| 218 | CL-75539 | CDRH3(IMGT) | Amino acid sequence of CDRH3 | ARRGFGEPFDY |
| | | | using IMGT | |
| 219 | CL-75539 | CDRH1 | Amino acid sequence of CDRH1 | SYAIH |
| | | (KABAT) | using Kabat | |
| 220 | CL-75539 | CDRH2 | Amino acid sequence of CDRH2 | WINVGNGKTKFSQKLQG |
| | | (KABAT) | using Kabat | |
| 221 | CL-75539 | CDRH3 | Amino acid sequence of CDRH3 | RGFGEPFDY |
| | | (KABAT) | using Kabat | |
| 222 | CL-75539 | Heavy chain | Amino acid sequence of V_{H} | QVQLVQSGAEVKKPGASVKVSCKASGYTFTSYAIHWVRQAPGQRLEWMGWINVGNG |
| | | variable | | KTKFSQKLQGRITITRDTSASTTYMELNSLRSEDTAVFYCARRGFGEPFDYWGQGTLVTV |
| | | region | | SS |
| 223 | CL-75539 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AAGTTTCCTGCAAGGCTTCTGGATACACCTTCACTAGCTATGCTATCCATTGGGTGCG |
| | | region | | CCAGGCCCCCGGACAAAGGCTTGAGTGGATGGGATGGATCAACGTTGGCAATGGTA |
| | | | | AAACAAAATTTTCACAGAAGTTACAGGGCAGAATCACCATTACCAGGGACACATCCG |
| | | | | CGAGCACAACCTACATGGAGCTGAACAGCCTGAGATCTGAAGACACGGCTGTGTTT |
| | | | | TACTGTGCGAGAAGGGGGTTCGGGGAGCCATTTGACTACTGGGGCCAGGGAACCC |
| | | | | TGGTCACCGTCTCCTCA |
| 224 | CL-75539 | Full heavy | Amino acid sequence heavy | QVQLVQSGAEVKKPGASVKVSCKASGYTFTSYAIHWVRQAPGQRLEWMGWINVGNG |
| | | chain | chain | KTKFSQKLQGRITITRDTSASTTYMELNSLRSEDTAVFYCARRGFGEPFDYWGQGTLVTV |
| | | sequence | | SSASTKGPSVFPLAPCSRSTSESTAALGCLVKDYFPEPVTVSWNSGALTSGVHTFPAVLQS |
| | | | | SGLYSLSSVVTVPSSSLGTKTYTCNVDHKPSNTKVDKRVESKYGPPCPPCPAPEFEGGPSV |
| | | | | FLFPPKPKDTLMISRTPEVTCVVVDVSQEDPEVQFNWYVDGVEVHNAKTKPREEQFNS |
| | | | | TYRVVSVLTVLHQDWLNGKEYKCKVSNKGLPSSIEKTISKAKGQPREPQVYTLPPSQEEM |
| | | | | TKNQVSLTCLVKGFYPSDIAVEWESNGQPENNYKTTPPVLDSDGSFFLYSRLTVDKSRW |
| | | | | QEGNVFSCSVMHEALHNHYTQKSLSLSLGK |
| 225 | CL-75539 | CDRL1(IMGT) | Amino acid sequence of CDRL1 | QSISSN |
| | | | using IMGT | |
| — | CL-75539 | CDRL2(IMGT) | Amino acid sequence of CDRL2 | GTS |
| | | | using IMGT | |
| 227 | CL-75539 | CDRL3(IMGT) | Amino acid sequence of CDRL3 | QQYNIWPFT |
| | | | using IMGT | |
| 228 | CL-75539 | CDRL1 | Amino acid sequence of CDRL1 | RASQSISSNLA |
| | | (KABAT) | using KABAT | |
| 229 | CL-75539 | CDRL2 | Amino acid sequence of CDRL2 | GTSTRAT |
| | | (KABAT) | using KABAT | |
| 230 | CL-75539 | CDRL3 | Amino acid sequence of CDRL3 | QQYNIWPFT |
| | | (KABAT) | using KABAT | |
| 231 | CL-75539 | Light chain | Amino acid sequence of V_{L} | EIVMTQSPATLSVSPGERATLSCRASQSISSNLAWYQQKPGQAPRLLIYGTSTRATGIPAR |
| | | variable | | FSGSGSGTEFTLTISTLQSEDFAVYYCQQYNIWPFTFGPGTKVDIK |
| | | region | | |
| 232 | CL-75539 | Light chain | Amino acid sequence of V_{L} | GAAATAGTGATGACGCAGTCTCCAGCCACCCTGTCTGTGTCTCCAGGGGAAAGAGC |
| | | variable | | CACCCTCTCCTGCAGGGCCAGTCAGAGTATTAGTAGCAACTTAGCCTGGTACCAACA |
| | | region | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAA |
| 233 | CL-75539 | Full light | Amino acid sequence light chain | EIVMTQSPATLSVSPGERATLSCRASQSISSNLAWYQQKPGQAPRLLIYGTSTRATGIPAR |
| | | chain | | FSGSGSGTEFTLTISTLQSEDFAVYYCQQYNIWPFTFGPGTKVDIKRTVAAPSVFIFPPSDE |
| | | sequence | | QLKSGTASVVCLLNNFYPREAKVQWKVDNALQSGNSQESVTEQDSKDSTYSLSSTLTLS |
| | | | | KADYEKHKVYACEVTHQGLSSPVTKSFNRGEC |
| 234 | CL-75565 | CDRH1(IMGT) | Amino acid sequence of CDRH1 | GYTFTSYA |
| | | | using IMGT | |
| 235 | CL-75565 | CDRH2(IMGT) | Amino acid sequence of CDRH2 | INAGNGNT |
| | | | using IMGT | |
| 236 | CL-75565 | CDRH3(IMGT) | Amino acid sequence of CDRH3 | ARRGFGEPFDY |
| | | | using IMGT | |
| 237 | CL-75565 | CDRH1 | Amino acid sequence of CDRH1 | SYAIH |
| | | (KABAT) | using Kabat | |
| 238 | CL-75565 | CDRH2 | Amino acid sequence of CDRH2 | WINAGNGNTKYSQKFQG |
| | | (KABAT) | using Kabat | |
| 239 | CL-75565 | CDRH3 | Amino acid sequence of CDRH3 | RGFGEPFDY |
| | | (KABAT) | using Kabat | |
| 240 | CL-75565 | Heavy chain | Amino acid sequence of V_{H} | QVQLVQSGAEVKKPGASVKVSCKASGYTFTSYAIHWVRQAPGQRLEWMGWINAGNG |
| | | variable | | NTKYSQKFQGRITITRDTSASTAYMELSSLRSEDTAVYYCARRGFGEPFDYWGQGTLVTV |
| | | region | | SS |
| 241 | CL-75565 | Heavy chain | Nucleotide sequence of V_{H} | CAGGTCCAGCTTGTGCAGTCTGGGGCTGAGGTGAAGAAGCCTGGGGCCTCAGTGA |
| | | variable | | AGGTTTCCTGCAAGGCTTCTGGATACACCTTCACTAGCTATGCTATCCATTGGGTGCG |
| | | region | | CCAGGCCCCCGGACAAAGGCTTGAGTGGATGGGATGGATCAACGCTGGCAATGGT |
| | | | | AACACAAAATATTCACAGAAGTTCCAGGGCAGAATCACCATTACCAGGGACACATCC |
| | | | | GCGAGCACAGCCTACATGGAGCTGAGCAGCCTGAGATCTGAAGACACGGCTGTGTA |
| | | | | TTACTGTGCGAGAAGGGGGTTCGGGGAGCCATTTGACTACTGGGGCCAGGGAACC |
| | | | | CTGGTCACCGTCTCCTCA |
| 242 | CL-75565 | Full heavy | Amino acid sequence heavy | QVQLVQSGAEVKKPGASVKVSCKASGYTFTSYAIHWVRQAPGQRLEWMGWINAGNG |
| | | chain | chain | NTKYSQKFQGRITITRDTSASTAYMELSSLRSEDTAVYYCARRGFGEPFDYWGQGTLVTV |
| | | sequence | | SSASTKGPSVFPLAPCSRSTSESTAALGCLVKDYFPEPVTVSWNSGALTSGVHTFPAVLQS |
| | | | | SGLYSLSSVVTVPSSSLGTKTYTCNVDHKPSNTKVDKRVESKYGPPCPPCPAPEFEGGPSV |
| | | | | FLFPPKPKDTLMISRTPEVTCVVVDVSQEDPEVQFNWYVDGVEVHNAKTKPREEQFNS |
| | | | | TYRVVSVLTVLHQDWLNGKEYKCKVSNKGLPSSIEKTISKAKGQPREPQVYTLPPSQEEM |
| | | | | TKNQVSLTCLVKGFYPSDIAVEWESNGQPENNYKTTPPVLDSDGSFFLYSRLTVDKSRW |
| | | | | QEGNVFSCSVMHEALHNHYTQKSLSLSLGK |
| 243 | CL-75565 | CDRL1(IMGT) | Amino acid sequence of CDRL1 | QSISSN |
| | | | using IMGT | |
| — | CL-75565 | CDRL2(IMGT) | Amino acid sequence of CDRL2 | GTS |
| | | | using IMGT | |
| 245 | CL-75565 | CDRL3(IMGT) | Amino acid sequence of CDRL3 | QQYNIWPFT |
| | | | using IMGT | |
| 246 | CL-75565 | CDRL1 | Amino acid sequence of CDRL1 | RASQSISSNLA |
| | | (KABAT) | using KABAT | |
| 247 | CL-75565 | CDRL2 | Amino acid sequence of CDRL2 | GTSTRAT |
| | | (KABAT) | using KABAT | |
| 248 | CL-75565 | CDRL3 | Amino acid sequence of CDRL3 | QQYNIWPFT |
| | | (KABAT) | using KABAT | |
| 249 | CL-75565 | Light chain | Amino acid sequence of V_{L} | EIVMTQSPATLSVSPGERATLSCRASQSISSNLAWYQQKPGQAPRLLIYGTSTRATGIPAR |
| | | variable | | FSGSGSGTEFTLTISTLQSEDFAVYYCQQYNIWPFTFGPGTKVDIK |
| | | region | | |
| 250 | CL-75565 | Light chain | Nucleic acid sequence of V_{L} | GAAATAGTGATGACGCAGTCTCCAGCCACCCTGTCTGTGTCTCCAGGGGAAAGAGC |
| | | variable | | CACCCTCTCCTGCAGGGCCAGTCAGAGTATTAGCAGCAACTTAGCCTGGTACCAACA |
| | | region | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAA |
| 251 | CL-75565 | Full light | Amino acid sequence light chain | EIVMTQSPATLSVSPGERATLSCRASQSISSNLAWYQQKPGQAPRLLIYGTSTRATGIPAR |
| | | chain | | FSGSGSGTEFTLTISTLQSEDFAVYYCQQYNIWPFTFGPGTKVDIKRTVAAPSVFIFPPSDE |
| | | sequence | | QLKSGTASVVCLLNNFYPREAKVQWKVDNALQSGNSQESVTEQDSKDSTYSLSSTLTLS |
| | | | | KADYEKHKVYACEVTHQGLSSPVTKSFNRGEC |
| 252 | CL-75714 | CDRH1(IMGT) | Amino acid sequence of CDRH1 | GYTFTTYA |
| | | | using IMGT | |
| 253 | CL-75714 | CDRH2(IMGT) | Amiino acid sequence of CDRH2 | INAGNGRT |
| | | | using IMGT | |
| 254 | CL-75714 | CDRH3(IMGT) | Amino acid sequence of CDRH3 | ARRGFGEPFDQ |
| | | | using IMGT | |
| 255 | CL-75714 | CDRH1 | Amino acid sequence of CDRH1 | TYAIH |
| | | (KABAT) | using Kabat | |
| 256 | CL-75714 | CDRH2 | Amino acid sequence of CDRH2 | WINAGNGRTEYSEKFQG |
| | | (KABAT) | using Kabat | |
| 257 | CL-75714 | CDRH3 | Amino acid sequence of CDRH3 | RGFGEPFDQ |
| | | (KABAT) | using Kabat | |
| 258 | CL-75714 | Heavy chain | Amino acid sequence of V_{H} | QVHLVQSGAEVKKPGASVKVSCKTSGYTFTTYAIHWVRQAPGQGLEWMGWINAGNG |
| | | variable | | RTEYSEKFQGRVTITRDTSASTVYMDLSSLRSGDTAVYYCARRGFGEPFDQWGQGTLVT |
| | | region | | VSS |
| 259 | CL-75714 | Heavy chain | Nucleotide sequence of V_{H} | CAGGTCCACCTTGTGCAGTCTGGGGCTGAGGTGAAGAAGCCTGGGGCCTCAGTGAA |
| | | variable | | GGTGTCCTGCAAGACTTCTGGATACACCTTCACCACCTATGCTATTCATTGGGTGCGC |
| | | region | | CAGGCCCCCGGACAAGGGCTTGAGTGGATGGGATGGATCAACGCTGGCAATGGTA |
| | | | | GAACAGAATATTCAGAGAAGTTTCAGGGCAGAGTCACCATTACCAGGGACACTTCC |
| | | | | GCGAGTACAGTCTACATGGACCTGAGCAGCCTGAGATCTGGAGACACGGCTGTGTA |
| | | | | TTACTGTGCGAGAAGGGGATTCGGGGAGCCATTTGACCAATGGGGCCAGGGAACC |
| | | | | CTGGTCACCGTCTCCTCA |
| 260 | CL-75714 | Full heavy | Amino acid sequence heavy | QVHLVQSGAEVKKPGASVKVSCKTSGYTFTTYAIHWVRQAPGQGLEWMGWINAGNG |
| | | chain | chain | RTEYSEKFQGRVTITRDTSASTVYMDLSSLRSGDTAVYYCARRGFGEPFDQWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 261 | CL-75714 | CDRL1(IMGT) | Amino acid sequence of CDRL1 | QSVSSN |
| | | | using IMGT | |
| — | CL-75714 | CDRL2(IMGT) | Amino acid sequence of CDRL2 | GAS |
| | | | using IMGT | |
| 263 | CL-75714 | CDRL3(IMGT) | Amino acid sequence of CDRL3 | QQYNNWPFI |
| | | | using IMGT | |
| 264 | CL-75714 | CDRL1 | Amino acid sequence of CDRL1 | RASQSVSSNLA |
| | | (KABAT) | using KABAT | |
| 265 | CL-75714 | CDRL2 | Amino acid sequence of CDRL2 | GASTRAT |
| | | (KABAT) | using KABAT | |
| 266 | CL-75714 | CDRL3 | Amino acid sequence of CDRL3 | QQYNNWPFI |
| | | (KABAT) | using KABAT | |
| 267 | CL-75714 | Light chain | Amino acid sequence of V_{L} | EIVMTQSPATLSVSPGERATLSCRASQSVSSNLAWYQHQPGQAPRLLIYGASTRATGFP |
| | | variable | | PRFSGSGSGTDFTLTINSLQSEDFAVYYCQQYNNWPFIFGPGTKLDIT |
| | | region | | |
| 268 | CL-75714 | Light chain | Nucleic acid sequence of V_{L} | GAAATAGTGATGACGCAGTCTCCAGCCACCCTGTCTGTGTCTCCAGGGGAAAGAGC |
| | | variable | | CACCCTCTCCTGCAGGGCCAGTCAGAGTGTTAGCAGCAACTTAGCCTGGTACCAGCA |
| | | region | | CCAACCTGGCCAGGCTCCCAGGCTCCTCATCTATGGTGCATCCACCAGGGCCACTGG |
| | | | | TTTCCCACCCAGGTTCAGTGGCAGTGGGTCTGGGACAGACTTCACTCTCACCATCAA |
| | | | | CAGCCTGCAGTCTGAAGATTTTGCAGTTTATTACTGTCAGCAGTATAATAACTGGCCT |
| | | | | TTCATTTTCGGCCCTGGGACCAAACTGGATATCACA |
| 269 | CL-75714 | Full light | Amino acid sequence light chain | EIVMTQSPATLSVSPGERATLSCRASQSVSSNLAWYQHQPGQAPRLLIYGASTRATGFP |
| | | chain | | PRFSGSGSGTDFTLTINSLQSEDFAVYYCQQYNNWPFIFGPGTKLDITRTVAAPSVFIFPP |
| | | sequence | | SDEQLKSGTASVVCLLNNFYPREAKVQWKVDNALQSGNSQESVTEQDSKDSTYSLSSTL |
| | | | | TLSKADYEKHKVYACEVTHQGLSSPVTKSFNRGEC |
| 270 | CL-58722 | CDRH1(IMGT) | Amino acid sequence of CDRH1 | GFTFSDYY |
| | | | using IMGT | |
| 271 | CL-58722 | CDRH2(IMGT) | Amino acid sequence of CDRH2 | ISISGSTI |
| | | | using IMGT | |
| 272 | CL-58722 | CDRH3(IMGT) | Amino acid sequence of CDRH3 | ARRSSGWYDY |
| | | | using IMGT | |
| 273 | CL-58722 | CDRH1 | Amino acid sequence of CDRH1 | DYYMS |
| | | (KABAT) | using Kabat | |
| 274 | CL-58722 | CDRH2 | Amino acid sequence of CDRH2 | YISISGSTIYYADSVKG |
| | | (KABAT) | using Kabat | |
| 275 | CL-58722 | CDRH3 | Amino acid sequence of CDRH3 | RSSGWYDY |
| | | (KABAT) | using Kabat | |
| 276 | CL-58722 | Heavy chain | Amino acid sequence of V_{H} | QVQLVESGGGLVKPGGSLRLSCAASGFTFSDYYMSWIRQAPGKGLEWVSYISISGSTIYY |
| | | variable | | ADSVKGRFTISRDNAKNSLFLQMNSLRAEDTAVYYCARRSSGWYDYWGQGTLVTVSS |
| | | region | | |
| 277 | CL-58722 | Heavy chain | Nucleotide sequence of V_{H} | CAGGTGCAACTGGTGGAGTCTGGGGGAGGCTTGGTCAAGCCTGGAGGGTCCCTGA |
| | | variable | | GACTCTCCTGTGCAGCCTCTGGATTCACCTTCAGTGACTACTACATGAGCTGGATCCG |
| | | region | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | AGAACTCACTGTTTCTGCAAATGAACAGCCTGAGAGCCGAGGACACGGCCGTGTAT |
| | | | | TACTGTGCGAGAAGAAGCAGTGGCTGGTACGACTACTGGGGCCAGGGAACCCTGG |
| | | | | TCACCGTCTCCTCA |
| 278 | CL-58722 | Full heavy | Amino acid sequence heavy | QVQLVESGGGLVKPGGSLRLSCAASGFTFSDYYMSWIRQAPGKGLEWVSYISISGSTIYY |
| | | chain | chain | ADSVKGRFTISRDNAKNSLFLQMNSLRAEDTAVYYCARRSSGWYDYWGQGTLVTVSSA |
| | | sequence | | STKGPSVFPLAPCSRSTSESTAALGCLVKDYFPEPVTVSWNSGALTSGVHTFPAVLQSSGL |
| | | | | YSLSSVVTVPSSSLGTKTYTCNVDHKPSNTKVDKRVESKYGPPCPPCPAPEFEGGPSVFLF |
| | | | | PPKPKDTLMISRTPEVTCVVVDVSQEDPEVQFNWYVDGVEVHNAKTKPREEQFNSTYR |
| | | | | VVSVLTVLHQDWLNGKEYKCKVSNKGLPSSIEKTISKAKGQPREPQVYTLPPSQEEMTK |
| | | | | NQVSLTCLVKGFYPSDIAVEWESNGQPENNYKT |
| | | | | TPPVLDSDGSFFLYSRLTVDKSRWQEGNVFSCSVMHEALHNHYTQKSLSLSLGK |
| 279 | CL-58722 | CDRL1(IMGT) | Amino acid sequence of CDRL1 | QSVSSNY |
| | | | using IMGT | |
| — | CL-58722 | CDRL2(IMGT) | Amino acid sequence of CDRL2 | GAS |
| | | | using IMGT | |
| 281 | CL-58722 | CDRL2(IMGT) | Amino acid sequence of CDRL3 | HQYGSSPPT |
| | | | using IMGT | |
| 282 | CL-58722 | CDRL1 | Amino acid sequence of CDRL1 | RASQSVSSNYLA |
| | | (KABAT) | using KABAT | |
| 283 | CL-58722 | CDRL2 | Amino acid sequence of CDRL2 | GASIRAT |
| | | (KABAT) | using KABAT | |
| 284 | CL-58722 | CDRL3 | Amino acid sequence of CDRL3 | HQYGSSPPT |
| | | (KABAT) | using KABAT | |
| 285 | CL-58722 | Light chain | Amino acid sequence of V_{L} | EIVLTQSPGTLSLSPGERATLSCRASQSVSSNYLAWYQQKPGQAPRLLIYGASIRATGIPD |
| | | variable | | RFSGSGSGTDFTLTISRLEPEDFAVYSCHQYGSSPPTFGQGTKVEIK |
| | | region | | |
| 286 | CL-58722 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCCTGCAGGGCCAGTCAGAGTGTTAGCAGCAACTACTTAGCCTGGTACCAG |
| | | region | | CAGAAACCTGGCCAGGCTCCCAGGCTCCTCATCTATGGTGCATCCATCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTCCTGTCACCAGTATGGTAGCTCA |
| | | | | CCTCCGACGTTCGGCCAAGGGACCAAGGTGGAAATCAAAC |
| 287 | CL-58722 | Full light | Amino acid sequence light chain | EIVLTQSPGTLSLSPGERATLSCRASQSVSSNYLAWYQQKPGQAPRLLIYGASIRATGIPD |
| | | chain | | RFSGSGSGTDFTLTISRLEPEDFAVYSCHQYGSSPPTFGQGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 288 | CL-58835 | CDRH1(IMGT) | Amino acid sequence of CDRH1 | GFTFSDYY |
| | | | using IMGT | |
| 289 | CL-58835 | CDRH2(IMGT) | Amino acid sequence of CDRH2 | ISISGTTI |
| | | | using IMGT | |
| 290 | CL-58835 | CDRH3(IMGT) | Amino acid sequence of CDRH3 | ARRSSGWYDY |
| | | | using IMGT | |
| 291 | CL-58835 | CDRH1 | Amino acid sequence of CDRH1 | DYYMS |
| | | (KABAT) | using Kabat | |
| 292 | CL-58835 | CDRH2 | Amino acid sequence of CDRH2 | YISISGTTIYYADSVKG |
| | | (KABAT) | using Kabat | |
| 293 | CL-58835 | CDRH3 | Amino acid sequence of CDRH3 | RSSGWYDY |
| | | (KABAT) | using Kabat | |
| 294 | CL-58835 | Heavy chain | Amino acid sequence of V_{H} | QVQLVESGGGLVKPGGSLRLSCAASGFTFSDYYMSWIRQAPGKGLEWVSYISISGTTIYY |
| | | variable | | ADSVKGRFTISRDNAMDSLYLQMNSLRAEDTAVYYCARRSSGWYDYWGQGTLVTVSS |
| | | region | | |
| 295 | CL-58835 | Heavy chain | Nucleotide sequence of V_{H} | CAGGTGCAACTGGTGGAGTCTGGGGGAGGCTTGGTCAAGCCTGGAGGGTCCCTGA |
| | | variable | | GACTCTCCTGTGCAGCCTCTGGATTCACCTTCAGTGACTACTACATGAGTTGGATCCG |
| | | region | | CCAGGCTCCAGGGAAGGGGCTGGAGTGGGTTTCATACATTAGTATTAGTGGTACTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCA |
| 296 | CL-58835 | Full heavy | Amino acid sequence heavy | QVQLVESGGGLVKPGGSLRLSCAASGFTFSDYYMSWIRQAPGKGLEWVSYISISGTTIYY |
| | | chain | chain | ADSVKGRFTISRDNAMDSLYLQMNSLRAEDTAVYYCARRSSGWYDYWGQGTLVTVSS |
| | | sequence | | ASTKGPSVFPLAPCSRSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPPCPPCPAPEFEGGPSVF |
| | | | | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALH NHYTQKSLSLSLGK |
| 297 | CL-58835 | CDRL1(IMGT) | Amino acid sequence of CDRL1 | QRVVYRY |
| | | | using IMGT | |
| — | CL-58835 | CDRL2(IMGT) | Amino acid sequence of CDRL2 | GAF |
| | | | using IMGT | |
| 299 | CL-58835 | CDRL3(IMGT) | Amino acid sequence of CDRL3 | HQYGSSPPT |
| | | | using IMGT | |
| 300 | CL-58835 | CDRL1 | Amino acid sequence of CDRL1 | WASQRVVYRYLA |
| | | (KABAT) | using KABAT | |
| 301 | CL-58835 | CDRL2 | Amino acid sequence of CDRL2 | GAFNRAT |
| | | (KABAT) | using KABAT | |
| 302 | CL-58835 | CDRL3 | Amino acid sequence of CDRL3 | HQYGSSPPT |
| | | (KABAT) | using KABAT | |
| 303 | CL-58835 | Light chain | Amino acid sequence of V_{L} | EIVLTQSPGTLSLSPGERATLSCWASQRVVYRYLAWYQRKPGQAPRLLIYGAFNRATGIP |
| | | variable | | DRFSGSGSGTDFSLTISRLEPEDFAVYYCHQYGSSPPTFGQGTKVEIK |
| | | region | | |
| 304 | CL-58835 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCTTGTTGGGCCAGTCAGAGGGTTGTTTACAGATACTTAGCCTGGTACCAG |
| | | region | | CGGAAACCTGGCCAGGCTCCCAGACTTCTCATTTATGGTGCATTCAACAGGGCCACT |
| | | | | GGCATCCCAGACAGGTTCAGTGGCAGTGGGTCTGGGACAGACTTCAGTCTCACTAT |
| | | | | CAGTAGACTGGAGCCTGAGGATTTTGCAGTTTATTACTGTCACCAATATGGTAGTTC |
| | | | | ACCACCGACGTTCGGCCAAGGGACCAAGGTGGAAATCAAA |
| 305 | CL-58835 | Full light | Amino acid sequence light chain | EIVLTQSPGTLSLSPGERATLSCWASQRVVYRYLAWYQRKPGQAPRLLIYGAFNRATGIP |
| | | chain | | DRFSGSGSGTDFSLTISRLEPEDFAVYYCHQYGSSPPTFGQGTKVEIKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 306 | CL-58756 | CDRH1(IMGT) | Amino acid sequence of CDRH1 | GFTFSDYY |
| | | | using IMGT | |
| 307 | CL-58756 | CDRH2(IMGT) | Amino acid sequence of CDRH2 | ISISGSTI |
| | | | using IMGT | |
| 308 | CL-58756 | CDRH3(IMGT) | Amino acid sequence of CDRH3 | ARRSSGWYDY |
| | | | using IMGT | |
| 309 | CL-58756 | CDRH1 | Amino acid sequence of CDRH1 | DYYMS |
| | | (KABAT) | using Kabat | |
| 310 | CL-58756 | CDRH2 | Amino acid sequence of CDRH2 | YISISGSTIYYADSVKG |
| | | (KABAT) | using Kabat | |
| 311 | CL-58756 | CDRH3 | Amino acid sequence of CDRH3 | RSSGWYDY |
| | | (KABAT) | using Kabat | |
| 312 | CL-58756 | Heavy chain | Amino acid sequence of V_{H} | QVQLVESGGGLVKPGGSLRLSCAASGFTFSDYYMSWIRQAPGKGLEWVSYISISGSTIYY |
| | | variable | | ADSVKGRFTISRDNAKNSLYLQMNSLRAEDTAVYYCARRSSGWYDYWGQGTLVTVSS |
| | | region | | |
| 313 | CL-58756 | Heavy chain | Nucleotide sequence of V_{H} | CAGGTGCAGCTGGTGGAGTCTGGGGGAGGCTTGGTCAAGCCTGGAGGGTCCCTGA |
| | | variable | | GACTCTCCTGTGCAGCCTCTGGATTCACCTTCAGTGACTACTACATGAGCTGGATCCG |
| | | region | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACCGCCA |
| | | | | AGAACTCACTGTATCTGCAAATGAACAGCCTGAGAGCCGAGGACACGGCCGTGTAT |
| | | | | TACTGTGCGAGAAGAAGCAGTGGCTGGTACGACTACTGGGGCCAGGGAACCCTGG |
| | | | | TCACCGTCTCCTCA |
| 314 | CL-58756 | Full heavy | Amino acid sequence heavy | QVQLVESGGGLVKPGGSLRLSCAASGFTFSDYYMSWIRQAPGKGLEWVSYISISGSTIYY |
| | | chain | chain | ADSVKGRFTISRDNAKNSLYLQMNSLRAEDTAVYYCARRSSGWYDYWGQGTLVTVSSA |
| | | sequence | | STKGPSVFPLAPCSRSTSESTAALGCLVKDYFPEPVTVSWNSGALTSGVHTFPAVLQSSGL |
| | | | | YSLSSVVTVPSSSLGTKTYTCNVDHKPSNTKVDKRVESKYGPPCPPCPAPEFEGGPSVFLF |
| | | | | PPKPKDTLMISRTPEVTCVVVDVSQEDPEVQFNWYVDGVEVHNAKTKPREEQFNSTYR |
| | | | | VVSVLTVLHQDWLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK |
| 315 | CL-58756 | CDRL1(IMGT) | Amino acid sequence of CDRL1 | QSVSSNY |
| | | | using IMGT | |
| — | CL-58756 | CDRL2(IMGT) | Amino acid sequence of CDRL2 | GAS |
| | | | using IMGT | |
| 317 | CL-58756 | CDRL3(IMGT) | Amino acid sequence of CDRL3 | HQYGSSPPT |
| | | | using IMGT | |
| 318 | CL-58756 | CDRL1 | Amino acid sequence of CDRL1 | RASQSVSSNYLA |
| | | (KABAT) | using KABAT | |
| 319 | CL-58756 | CDRL2 | Amino acid sequence of CDRL2 | GASIRAT |
| | | (KABAT) | using KABAT | |
| 320 | CL-58756 | CDRL3 | Amino acid sequence of CDRL3 | HQYGSSPPT |
| | | (KABAT) | using KABAT | |
| 321 | CL-58756 | Light chain | Amino acid sequence of V_{L} | EIVLTQSPGTLSLSPGERATLSCRASQSVSSNYLAWYQQKPGQAPRLLIYGASIRATGIPD |
| | | variable | | RFSGSGSGTDFTLTISRLEPEDFAVYCCHQYGSSPPTFGQGTKVEIK |
| | | region | | |
| 322 | CL-58756 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCCTGCAGGGCCAGTCAGAGTGTTAGCAGCAACTACTTAGCCTGGTACCAG |
| | | region | | CAGAAACCTGGCCAGGCTCCCAGGCTCCTCATCTATGGTGCATCCATCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGCTGTCACCAGTATGGTAGCTCA |
| | | | | CCTCCGACGTTCGGCCAAGGGACCAAGGTGGAAATCAAA |
| 323 | CL-58756 | Full light | Amio acid sequence light chain | EIVLTQSPGTLSLSPGERATLSCRASQSVSSNYLAWYQQKPGQAPRLLIYGASIRATGIPD |
| | | chain | | RFSGSGSGTDFTLTISRLEPEDFAVYCCHQYGSSPPTFGQGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 324 | CL-58650 | CDRH1(IMGT) | Amino acid sequence of CDRH1 | GFTFSDYF |
| | | | using IMGT | |
| 325 | CL-58650 | CDRH2(IMGT) | Amino acid sequence of CDRH2 | ISISGSTI |
| | | | using IMGT | |
| 326 | CL-58650 | CDRH3(IMGT) | Amino acid sequence of CDRH3 | ARRTSGWYDF |
| | | | using IMGT | |
| 327 | CL-58650 | CDRH1 | Amino acid sequence of CDRH1 | DYFMS |
| | | (KABAT) | using Kabat | |
| 328 | CL-58650 | CDRH2 | Amino acid sequence of CDRH2 | YISISGSTIYYADSVKG |
| | | (KABAT) | using Kabat | |
| 329 | CL-58650 | CDRH3 | Amino acid sequence of CDRH3 | RTSGWYDF |
| | | (KABAT) | using Kabat | |
| 330 | CL-58650 | Heavy chain | Amino acid sequence of V_{H} | QVQLVESGGGLVKPGGSLRLSCAASGFTFSDYFMSWIRQAPGKGLEWVSYISISGSTIYY |
| | | variable | | ADSVKGRFTISRDNARNSLFLQMNSLRAEDTAIYYCARRTSGWYDFWGQGTLVTVSS |
| | | region | | |
| 331 | CL-58650 | Heavy chain | Nucleotide sequence of V_{H} | CAGGTGCAGC TGGTGGAGTC TGGGGGAGGC TTGGTCAAGC CTGGAGGGTC |
| | | variable | | CCTGCGACTC TCCTGTGCAG CCTCTGGATT |
| | | region | | CACCTTCAGT GACTACTTCA TGAGCTGGAT CCGCCAGGCT CCAGGGAAGG |
| | | | | GGCTGGAGTG GGTTTCGTAC ATTAGTATTA |
| | | | | GTGGTAGTAC CATATACTAC GCAGACTCTG TGAAGGGCCG ATTCACCATC |
| | | | | TCCAGGGACA ACGCCAGGAA CTCACTGTTT |
| | | | | CTGCAAATGA ACAGCCTGAG AGCCGAGGAC ACGGCCATCT ATTACTGTGC |
| | | | | GAGAAGAACC AGTGGCTGGT ACGACTTCTG |
| | | | | GGGCCAGGGA ACCCTGGTCA CCGTCTCCTC A |
| 332 | CL-58650 | Full heavy | Amino acid sequence heavy | QVQLVESGGGLVKPGGSLRLSCAASGFTFSDYFMSWIRQAPGKGLEWVSYISISGSTIYY |
| | | chain | chain | ADSVKGRFTISRDNARNSLFLQMNSLRAEDTAIYYCARRTSGWYDFWGQGTLVTVSSA |
| | | sequence | | STKGPSVFPLAPCSRSTSESTAALGCLVKDYFPEPVTVSWNSGALTSGVHTFPAVLQSSGL |
| | | | | YSLSSVVTVPSSSLGTKTYTCNVDHKPSNTKVDKRVESKYGPPCPPCPAPEFEGGPSVFLF |
| | | | | PPKPKDTLMISRTPEVTCVVVDVSQEDPEVQFNWYVDGVEVHNAKTKPREEQFNSTYR |
| | | | | VVSVLTVLHQDWLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK |
| 333 | CL-58650 | CDRL1(IMGT) | Amino acid sequence of CDRL1 | QSVSYSY |
| | | | using IMGT | |
| — | CL-58650 | CDRL2(IMGT) | Amino acid sequence of CDRL2 | GAS |
| | | | using IMGT | |
| 335 | CL-58650 | CDRL3(IMGT) | Amino acid sequence of CDRL3 | HQYGSSPPT |
| | | | using IMGT | |
| 336 | CL-58650 | CDRL1 | Amino acid sequence of CDRL1 | RASQSVSYSYLA |
| | | (KABAT) | using KABAT | |
| 337 | CL-58650 | CDRL2 | Amino acid sequence of CDRL2 | GASSRAT |
| | | (KABAT) | using KABAT | |
| 338 | CL-58650 | CDRL3 | Amino acid sequence of CDRL3 | HQYGSSPPT |
| | | (KABAT) | using KABAT | |
| 339 | CL-58650 | Light chain | Amino acid sequence of V_{L} | EIVLTQSPGTLSLSPGERATLSCRASQSVSYSYLAWYQQKPGQAPRLLIYGASSRATGIPD |
| | | variable | | RFSGSGSGTDFTLTISRLEPEDFAVYCCHQYGSSPPTFGQGTKVEIK |
| | | region | | |
| 340 | CL-58650 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCCTGCAGGGCCAGTCAGAGTGTTAGCTACAGCTACTTAGCCTGGTACCAG |
| | | region | | CAGAAACCTGGCCAGGCTCCCAGGCTCCTCATCTATGGTGCATCCAGCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGTTGTCACCAGTATGGTAGTTCA |
| | | | | CCTCCGACGTTCGGCCAAGGGACCAAGGTGGAAATCAAA |
| 341 | CL-58650 | Full light | Amino acid sequence light chain | EIVLTQSPGTLSLSPGERATLSCRASQSVSYSYLAWYQQKPGQAPRLLIYGASSRATGIPD |
| | | chain | | RFSGSGSGTDFTLTISRLEPEDFAVYCCHQYGSSPPTFGQGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 342 | CL-58679 | CDRH1(IMGT) | Amino acid sequence of CDRH1 | GFTFSDFY |
| | | | using IMGT | |
| 343 | CL-58679 | CDRH2(IMGT) | Amino acid sequence of CDRH2 | ISISGTTI |
| | | | using IMGT | |
| 344 | CL-58679 | CDRH3(IMGT) | Amino acid sequence of CDRH3 | ARRTSGWYDF |
| | | | using IMGT | |
| 345 | CL-58679 | CDRH1 | Amino acid sequence of CDRH1 | DFYMS |
| | | (KABAT) | using Kabat | |
| 346 | CL-58679 | CDRH2 | Amino acid sequence of CDRH2 | YISISGTTIYYADSVKG |
| | | (KABAT) | using Kabat | |
| 347 | CL-58679 | CDRH3 | Amino acid sequence of CDRH3 | RTSGWYDF |
| | | (KABAT) | using Kabat | |
| 348 | CL-58679 | Heavy chain | Amino acid sequence of V_{H} | QVQLVESGGGLVKPGGSLRLSCAASGFTFSDFYMSWIRQAPGRGLEWVSYISISGTTIYY |
| | | variable | | ADSVKGRFTISRDNARNSLYLQMNSLRAEDTAVYYCARRTSGWYDFWGQG TLVTVSS |
| | | region | | |
| 349 | CL-58679 | Heavy chain | Nucleotide sequence of V_{H} | CAGGTGCAGCTGGTGGAGTCTGGGGGAGGCTTGGTCAAGCCTGGAGGGTC CCTTA |
| | | variable | | GACTCTCCTGTGCAGCCTCTGGATTCACCTTCAGTGACTTCTACATGAGCTGGATCCG |
| | | region | | CCAGGCTCCAGGGAGGGGGCTGGAGTGGGTTTCGTACATTAGTATTAGTGGTACTA |
| | | | | CCATATACTACGCAGACTCTGTGAAGGGCCGATTCACCATCTCCAGGGACAACGCCA |
| | | | | GGAACTCACTGTATCTGCAAATGAACAGCCTGAGAGCCGAGGACACGGCCGTGTAT |
| | | | | TACTGTGCGAGAAGAACCAGTGGCTGGTACGACTTCTGGGGCCAGGGAACCCTGGT |
| | | | | CACCGTCTCCTCA |
| 350 | CL-58679 | Full heavy | Amino acid sequence heavy | QVQLVESGGGLVKPGGSLRLSCAASGFTFSDFYMSWIRQAPGRGLEWVSYISISGTTIYY |
| | | chain | chain | ADSVKGRFTISRDNARNSLYLQMNSLRAEDTAVYYCARRTSGWYDFWGQG TLVTVSS |
| | | sequence | | ASTKGPSVFPLAPCSRSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPPCPPCPAPEFEGGPSVF |
| | | | | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 351 | CL-58679 | CDRL1(IMGT) | Amino acid sequence of CDRL1 | QSVSYSY |
| | | | using IMGT | |
| — | CL-58679 | CDRL2(IMGT) | Amino acid sequence of CDRL2 | GAS |
| | | | using IMGT | |
| 353 | CL-58679 | CDRL3(IMGT) | Amino acid sequence of CDRL3 | HQYGSSPPT |
| | | | using IMGT | |
| 354 | CL-58679 | CDRL1 | Amino acid sequence of CDRL1 | RASQSVSYSYLA |
| | | (KABAT) | using KABAT | |
| 355 | CL-58679 | CDRL2 | Amino acid sequence of CDRL2 | GASSRAT |
| | | (KABAT) | using KABAT | |
| 356 | CL-58679 | CDRL3 | Amino acid sequence of CDRL3 | HQYGSSPPT |
| | | (KABAT) | using KABAT | |
| 357 | CL-58679 | Light chain | Amino acid sequence of V_{L} | EIVLTQSPGTLSLSPGERATLSCRASQSVSYSYLAWYQQKPGQAPRLLIYGASSRATGIPD |
| | | variable | | RFSGSGSGTDFTLTISRLEPEDFAVYCCHQYGSSPPTFGQGTKVEMK |
| | | region | | |
| 358 | CL-58679 | Light chain | Nucleic acid sequence of V_{L} | GAAATTGTGTTGACGCAGTCTCCAGGCACCCTGTCTTTGTCTCCAGGGGAAAGAGCC |
| | | variable | | ACCCTCTCCTGCAGGGCCAGTCAGAGTGTTAGCTACAGCTACTTAGCCTGGTACCAG |
| | | region | | CAGAAACCTGGCCAGGCTCCCAGGCTCCTCATCTATGGTGCATCCAGCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGCTGTCACCAGTATGGTAGTTCA |
| | | | | CCTCCGACGTTCGGCCAAGGGACCAAGGTGGAAATGAAA |
| 359 | CL-58679 | Full light | Amino acid sequence light chain | EIVLTQSPGTLSLSPGERATLSCRASQSVSYSYLAWYQQKPGQAPRLLIYGASSRATGIPD |
| | | chain | | RFSGSGSGTDFTLTISRLEPEDFAVYCCHQYGSSPPTFGQGTKVEMKRTVAAPSVFIFPPS |
| | | sequence | | DEQLKSGTASVVCLLNNFYPREAKVQWKVDNALQSGNSQESVTEQDSKDSTYSLSSTLT |
| | | | | LSKADYEKHKVYACEVTHQGLSSPVTKSFNRGEC |
| 360 | CL-58680 | CDRH1(IMGT) | Amino acid sequence of CDRH1 | GFTFSDYY |
| | | | using IMGT | |
| 361 | CL-58680 | CDRH2(IMGT) | Amino acid sequence of CDRH2 | ISISGSTI |
| | | | using IMGT | |
| 362 | CL-58680 | CDRH3(IMGT) | Amino acid sequence of CDRH3 | ARRTSGWYDF |
| | | | using IMGT | |
| 363 | CL-58680 | CDRH1 | Amino acid sequence of CDRH1 | DYYMS |
| | | (KABAT) | using Kabat | |
| 364 | CL-58680 | CDRH2 | Amino acid sequence of CDRH2 | YISISGSTIYYADSVKG |
| | | (KABAT) | using Kabat | |
| 365 | CL-58680 | CDRH3 | Amino acid sequence of CDRH3 | RTSGWYDF |
| | | (KABAT) | using Kabat | |
| 366 | CL-58680 | Heavy chain | Amino acid sequence of V_{H} | QVQLVESGGGLVRPGGSLRLSCAASGFTFSDYYMSWIRQAPGKGLEWVSYISISGSTIYY |
| | | variable | | ADSVKGRFTISRDNARDSLYLQMNSLRAEDTAVYYCARRTSGWYDFWGQG TLVTVSS |
| | | region | | |
| 367 | CL-58680 | Heavy chain | Nucleotide sequence of V_{H} | CAGGTGCAGC TGGTGGAGTC TGGGGGAGGC TTGGTCAGGC CTGGAGGGTC CCT |
| | | variable | | GAGACTC TCCTGTGCAG CCTCTGGATT |
| | | region | | CACCTTCAGT GACTACTACA TGAGCTGGAT CCGCCAGGCT CCAGGGAAGG GGCT |
| | | | | GGAGTG GGTTTCGTAC ATTAGTATTA |
| | | | | GTGGTAGTAC CATATACTAC GCAGACTCTG TGAAGGGCCG ATTCACCATC TCCAG |
| | | | | GGACA ACGCCAGGGA CTCACTTTAT |
| | | | | CTGCAAATGA ACAGCCTGAG AGCCGAGGAC ACGGCCGTGT ATTACTGTGC GAGA |
| | | | | AGAACC AGTGGCTGGT ACGACTTCTG |
| | | | | GGGCCAGGGA ACCCTGGTCA CCGTCTCCTCA |
| 368 | CL-58680 | Full heavy | Amino acid sequence heavy | QVQLVESGGGLVRPGGSLRLSCAASGFTFSDYYMSWIRQAPGKGLEWVSYISISGTIYY |
| | | chain | chain | ADSVKGRFTISRDNARDSLYLQMNSLRAEDTAVYYCARRTSGWYDFWGQG TLVTVSS |
| | | sequence | | ASTKGPSVFPLAPCSRSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPPCPPCPAPEFEGGPSVF |
| | | | | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 369 | CL-58680 | CDRL1(IMGT) | Amino acid sequence of CDRL1 | QSVSYRY |
| | | | using IMGT | |
| — | CL-58680 | CDRL2(IMGT) | Amino acid sequence of CDRL2 | GAS |
| | | | using IMGT | |
| 371 | CL-58680 | CDRL3(IMGT) | Amino acid sequence of CDRL3 | HQYGSSPPT |
| | | | using IMGT | |
| 372 | CL-58680 | CDRL1 | Amino acid sequence of CDRL1 | RASQSVSYRYLA |
| | | (KABAT) | using KABAT | |
| 373 | CL-58680 | CDRL2 | Amino acid sequence of CDRL2 | GASSRAT |
| | | (KABAT) | using KABAT | |
| 374 | CL-58680 | CDRL3 | Amino acid sequence of CDRL3 | HQYGSSPPT |
| | | (KABAT) | using KABAT | |
| 375 | CL-58680 | Light chain | Amino acid sequence of V_{L} | EIVLTQSPGTLSLSPGERATLSCRASQSVSYRYLAWYQQKPGQAPRLLIYGASSRATGIPD |
| | | variable | | RFSGSGSGTDFTLTISRLEPEDFAVYCCHQYGSSPPTFGQGTKVEIK |
| | | region | | |
| 376 | CL-58680 | Light chain | Nucleic acid sequence of V_{L} | GAAATTGTGT TGACGCAGTC TCCAGGCACC CTGTCTTTGT CTCCAGGGGA AAGAG |
| | | variable | | CCACC CTCTCCTGTA GGGCCAGTCA |
| | | region | | GAGTGTTAGC TACAGGTACT TAGCCTGGTA CCAGCAGAAA CCTGGCCAGG CTCC |
| | | | | CAGGCT CCTCATCTAT GGTGCATCCA |
| | | | | GCAGGGCCAC TGGCATCCCA GACAGGTTCA GTGGCAGTGG GTCTGGGACA GACT |
| | | | | TCACTC TCACCATCAG CAGACTGGAG |
| | | | | CCTGAAGATT TTGCAGTGTA TTGCTGTCAC CAGTATGGTA GTTCACCTCC GACGTT |
| | | | | CGGC CAAGGGACCA AGGTGGAAAT |
| | | | | CAAA |
| 377 | CL-58680 | Full light | Amino acid sequence light chain | EIVLTQSPGTLSLSPGERATLSCRASQSVSYRYLAWYQQKPGQAPRLLIYGASSRATGIPD |
| | | chain | | RFSGSGSGTDFTLTISRLEPEDFAVYCCHQYGSSPPTFGQGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 378 | CL-58713 | CDRH1(IMGT) | Amino acid sequence of CDRH1 | GFTFSDYY |
| | | | using IMGT | |
| 379 | CL-58713 | CDRH2(IMGT) | Amino acid sequence of CDRH2 | ISISGITI |
| | | | using IMGT | |
| 380 | CL-58713 | CDRH3(IMGT) | Amino acid sequence of CDRH3 | ARRSSGWYDY |
| | | | using IMGT | |
| 381 | CL-58713 | CDRH1 | Amino acid sequence of CDRH1 | DYYMS |
| | | (KABAT) | using Kabat | |
| 382 | CL-58713 | CDRH2 | Amino acid sequence of CDRH2 | YISISGITIYYADSVKG |
| | | (KABAT) | using Kabat | |
| 382 | CL-58713 | CDRH3 | Amino acid sequence of CDRH3 | RSSGWYDY |
| | | (KABAT) | using Kabat | |
| 384 | CL-58713 | Heavy chain | Amino acid sequence of V_{H} | QVQLVESGGGLVKPGGSLRLSCAASGFTFSDYYMSWIRQAPGKGLEWVSYISISGITIYY |
| | | variable | | ADSVKGRFTISRDNARNSLYLQMNSLRAEDTAVYYCARRSSGWYDYWGQG TLVTVSS |
| | | region | | |
| 385 | CL-58713 | Heavy chain | Nucleotide sequence of V_{H} | CAAGTTCAGTTGGTTGAGTCTGGCGGCGGACTGGTTAAGCCTGGCGGATCTCTGAG |
| | | variable | | ACTGTCTTGTGCCGCCTCTGGCTTCACCTTCTCCGACTACTACATGTCCTGGATCAGA |
| | | region | | CAGGCCCCTGGCAAAGGCCTGGAATGGGTGTCCTACATCTCCATCTCCGGCATCACC |
| | | | | ATCTACTACGCCGACTCCGTGAAGGGCAGATTCACCATCTCCAGAGACAACGCCCGG |
| | | | | AACTCCCTGTACCTGCAGATGAACTCTCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTTTCTAGT |
| 386 | CL-58713 | Full heavy | Amino acid sequence heavy | QVQLVESGGGLVKPGGSLRLSCAASGFTFSDYYMSWIRQAPGKGLEWVSYISISGITIYY |
| | | chain | chain | ADSVKGRFTISRDNARNSLYLQMNSLRAEDTAVYYCARRSSGWYDYWGQG TLVTVSS |
| | | sequence | | ASTKGPSVFPLAPCSRSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPPCPPCPAPEFEGGPSVF |
| | | | | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 387 | CL-58713 | CDRL1(IMGT) | Amino acid sequence of CDRL1 | QSVSYNY |
| | | | using IMGT | |
| — | CL-58713 | CDRL2(IMGT) | Amino acid sequence of CDRL2 | GAS |
| | | | using IMGT | |
| 389 | CL-58713 | CDRL3(IMGT) | Amino acid sequence of CDRL3 | HQYGSSPPT |
| | | | using IMGT | |
| 390 | CL-58713 | CDRL1 | Amino acid sequence of CDRL1 | RASQSVSYNYLA |
| | | (KABAT) | using KABAT | |
| 391 | CL-58713 | CDRL2 | Amino acid sequence of CDRL2 | GASIRAT |
| | | (KABAT) | using KABAT | |
| 392 | CL-58713 | CDRL3 | Amino acid sequence of CDRL3 | HQYGSSPPT |
| | | (KABAT) | using KABAT | |
| 393 | CL-58713 | Light chain | Amino acid sequence of V_{L} | EIVLTQSPGTLSLSPGERATLSCRASQSVSYNYLAWYQQKPGQAPRLLIYGASIRATGIPD |
| | | variable | | RFSGSGSGTDFTLTISRLEPEDFAVYCCHQYGSSPPTFGQGTKVEIK |
| | | region | | |
| 394 | CL-58713 | Light chain | Nucleic acid sequence of V_{L} | GAAATTGTGCTGACTCAGTCCCCTGGCACACTGTCTTTGAGCCCTGGCGAGAGAGCT |
| | | variable | | ACCCTGTCCTGTAGAGCCTCTCAGTCCGTGTCCTACAACTACCTGGCCTGGTATCAGC |
| | | region | | AGAAGCCCGGCCAGGCTCCTAGACTGTTGATCTACGGCGCCTCCATCAGAGCCCACA |
| | | | | GGCATCCCTGATAGATTCTCCGGCTCTGGCTCTGGCACCGACTTCACCCTGACCATCT |
| | | | | CCAGACTGGAACCCGAGGACTTCGCCGTGTACTGCTGTCACCAGTACGGCTCTAGCC |
| | | | | CTCCTACCTTTGGACAGGGCACCAAGGTGGAAATCAAA |
| 395 | CL-58713 | Full light | Amino acid sequence light chain | EIVLTQSPGTLSLSPGERATLSCRASQSVSYNYLAWYQQKPGQAPRLLIYGASIRATGIPD |
| | | chain | | RFSGSGSGTDFTLTISRLEPEDFAVYCCHQYGSSPPTFGQGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 396 | Antibody A | CDRH1(IMGT) | Amino acid sequence of CDRH1 | GYTFTSYA |
| | | | using IMGT | |
| 397 | Antibody A | CDRH2(IMGT) | Amino acid sequence of CDRH2 | INPYNRGT |
| | | | using IMGT | |
| 398 | Antibody A | CDRH3(IMGT) | Amino acid sequence of CDRH3 | ARRPFGNAMDI |
| | | | using IMGT | |
| 399 | Antibody A | CDRH1 | Amino acid sequence of CDRH1 | SYAMH |
| | | (KABAT) | using Kabat | |
| 400 | Antibody A | CDRH2 | Amino acid sequence of CDRH2 | YINPYNRGTKYNENFKG |
| | | (KABAT) | using Kabat | |
| 401 | Antibody A | CDRH3 | Amino acid sequence of CDRH3 | RPFGNAMDI |
| | | (KABAT) | using Kabat | |
| 402 | Antibody A | Heavy chain | Amino acid sequence of V_{H} | QVQLVQSGAEVKKPGSSVKVSCKASGYTFTSYAMHWVRQAPGQGLEWMGYINPYND |
| | | variable | | GTKYNENFKGRVTITADESTSTAYMELSSLRSEDTAVYYCARRPFGNAMDIWGQGTLVT |
| | | region | | VSS |
| 403 | Antibody A | Full heavy | Amino acid sequence heavy | QVQLVQSGAEVKKPGSSVKVSCKASGYTFTSYAMHWVRQAPGQGLEWMGYINPYND |
| | | chain | chain of Antibody A | GTKYNENFKGRVTITADESTSTAYMELSSLRSEDTAVYYCARRPFGNAMDIWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |
| 404 | Antibody A | CDRL1(IMGT) | Amino acid sequence of CDRL1 | ENIYRN |
| | | | using IMGT | |
| — | Antibody A | CDRL2(IMGT) | Amino acid sequence of CDRL2 | AAT |
| | | | using IMGT | |
| 406 | Antibody A | CDRL3(IMGT) | Amino acid sequence of CDRL3 | QGIWGTPLT |
| | | | using IMGT | |
| 407 | Antibody A | CDRL1 | Amino acid sequence of CDRL1 | RSSENIYRNLA |
| | | (KABAT) | using KABAT | |
| 408 | Antibody A | CDRL2 | Amino acid sequence of CDRL2 | AATNLAD |
| | | (KABAT) | using KABAT | |
| 409 | Antibody A | CDRL3 | Amino acid sequence of CDRL3 | QGIWGTPLT |
| | | (KABAT) | using KABAT | |
| 410 | Antibody A | Light chain | Amino acid sequence of V_{L} | DIQMTQSPSSLSASVGDRVTITCRSSENIYRNLAWYQQKPGKAPKLLIYAATNLADGVPS |
| | | variable | | RFSGSGSGTDFTLTISSLQPEDFATYYCQGIWGTPLTFGGGTKVEIK |
| | | region | | |
| 411 | Antibody A | Full light | Amino acid sequence light chain | DIQMTQSPSSLSASVGDRVTITCRSSENIYRNLAWYQQKPGKAPKLLIYAATNLADGVPS |
| | | chain | of Antibody A | RFSGSGSGTDFTLTISSLQPEDFATYYCQGIWGTPLTFGGGTKVEIKRTVAAPSVFIFPPSD |
| | | sequence | | EQLKSGTASVVCLLNNFYPREAKVQWKVDNALQSGNSQESVTEQDSKDSTYSLSSTLTL |
| | | | | SKADYEKHKVYACEVTHQGLSSPVTKSFNRGEC |
| 412 | Antibody B | CDRH1(IMGT) | Amino acid sequence of CDRH1 | GFTFSSYV |
| | | | using IMGT | |
| 413 | Antibody B | CDRH2(IMGT) | Amino acid sequence of CDRH2 | IRLETHGYAA |
| | | | using IMGT | |
| 414 | Antibody B | CDRH3(IMGT) | Amino acid sequence of CDRH3 | ARVERSKSGFDN |
| | | | using IMGT | |
| 415 | Antibody B | CDRH1 | Amino acid sequence of CDRH1 | SYVVH |
| | | (KABAT) | using Kabat | |
| 416 | Antibody B | CDRH2 | Amino acid sequence of CDRH2 | RIRLETHGYAAEYAASVKG |
| | | (KABAT) | using Kabat | |
| 417 | Antibody B | CDRH3 | Amino acid sequence of CDRH3 | VERSKSGFDN |
| | | (KABAT) | using Kabat | |
| 418 | Antibody B | Heavy chain | Amino acid sequence of V_{H} | QVQLVESGGGLVKPGGSLRLSCAASGFTFSSYVVHWVRQAPGKGLEWVGRIRLETHGY |
| | | variable | | AAEYAASVKGRFTISRDDSKNTLYLQMNSLKTEDTAVYYCARVERSKSGFDNWGQGTL |
| | | region | | VTVSS |
| 419 | Antibody B | Full heavy | Amino acid sequence heavy | QVQLVESGGGLVKPGGSLRLSCAASGFTFSSYVVHWVRQAPGKGLEWVGRIRLETHGY |
| | | chain | chain of Antibody B | AAEYAASVKGRFTISRDDSKNTLYLQMNSLKTEDTAVYYCARVERSKSGFDNWGQGTL |
| | | sequence | | VTVSSASTKGPSVFPLAPSSKSTSGGTAALGCLVKDYFPEPVTVSWNSGALTSGVHTFPA |
| | | | | VLQSSGLYSLSSVVTVPSSSLGTQTYICNVNHKPSNTKVDKRVEPKSCDKTHTCPPCPAPE |
| | | | | LLGGPSVFLFPPKPKDTLMISRTPEVTCVVVDVSHEDPEVKFNWYVDGVEVHNAKTKPR |
| | | | | EEQYNSTYRVVSVLTVLHQDWLNGKEYKCKVSNKALPAPIEKTISKAKGQPREPQVYTLP |
| | | | | PSREEMTKNQVSLTCLVKGFYPSDIAVEWESNGQPENNYKTTPPVLDSDGSFFLYSKLTV |
| | | | | DKSRWQQGNVFSCSVMHEALHNHYTQKSLSLSPGK |
| 420 | Antibody B | CDRL1(IMGT) | Amino acid sequence of CDRL1 | SSNIGAGYS |
| | | | using IMGT | |
| — | Antibody B | CDRL2(IMGT) | Amino acid sequence of CDRL2 | GQS |
| | | | using IMGT | |
| 422 | Antibody B | CDRL2(IMGT) | Amino acid sequence of CDRL3 | QSWDSSQTLVV |
| | | | using IMGT | |
| 423 | Antibody B | CDRL1 | Amino acid sequence of CDRL1 | TGSSSNIGAGYSVH |
| | | (KABAT) | using KABAT | |
| 424 | Antibody B | CDRL2 | Amino acid sequence of CDRL2 | GQSERPS |
| | | (KABAT) | using KABAT | |
| 425 | Antibody B | CDRL2 | Amino acid sequence of CDRL3 | QSWDSSQTLVV |
| | | (KABAT) | using KABAT | |
| 426 | Antibody B | Light chain | Amino acid sequence of V_{L} | QSVLTQPPSVSGAPGQRVTISCTGSSSNIGAGYSVHWYQQLPGTAPKLLIYGQSERPSGV |
| | | variable | | PDRFSGSKSGTSASLAITGLQAEDEADYYCQSWDSSQTLVVFGGGTKLTVL |
| | | region | | |
| 427 | Antibody B | Full light | Amino acid sequence light chain | QSVLTQPPSVSGAPGQRVTISCTGSSSNIGAGYSVHWYQQLPGTAPKLLIYGQSERPSGGV |
| | | chain | Antibody B | PDRFSGSKSGTSASLAITGLQAEDEADYYCQSWDSSQTLVVFGGGTKLTVLGQPKAAPS |
| | | sequence | | VTLFPPSSEELQANKATLVCLISDFYPGAVTVAWKADSSPVKAGVETTTPSKQSNNKYAA |
| | | | | SSYLSLTPEQWKSHRSYSCQVTHEGSTVEKTVAPTECS |
| 428 | Human IgG1 | IGHG1*01 | Human Heavy Chain Constant | gcctccaccaagggcccatcggtcttccccctggcaccctcctccaagagcacctctgggggcacagcggccct |
| | constant | | Region (IGHG1*01) Nucleotide | gggctgcctggtcaaggactacttccccgaaccggtgacggtgtcgtggaactcaggcgccctgaccagcggc |
| | region | | Sequence | gtgcacaccttcccggctgtcctacagtcctcaggactctactccctcagcagcgtggtgaccgtgccctccagc |
| | | | | agcttgggcacccagacctacatctgcaacgtgaatcacaagcccagcaacaccaaggtggacaagaaagtt |
| | | | | gagcccaaatcttgtgacaaaactcacacatgcccaccgtgcccagcacctgaactcctggggggaccgtcag |
| | | | | tcttcctcttccccccaaaacccaaggacaccctcatgatctcccggacccctgaggtcacatgcgtggtggtgg |
| | | | | acgtgagccacgaagaccctgaggtcaagttcaactggtacgtggacggcgtggaggtgcataatgccaaga |
| | | | | caaagccgcgggaggagcagtacaacagcacgtaccgggtggtcagcgtcctcaccgtcctgcaccaggact |
| | | | | ggctgaatggcaaggagtacaagtgcaaggtctccaacaaagccctcccagcccccatcgagaaaaccatct |
| | | | | ccaaagccaaagggcagccccgagaaccacaggtgtacaccctgcccccatcccgggatgagctgaccaaga |
| | | | | accaggtcagcctgacctgcctggtcaaaggcttctatcccagcgacatcgccgtggagtgggagagcaatgg |
| | | | | gcagccggagaacaactacaagaccacgcctcccgtgctggactccgacggctccttcttcctctacagcaagc |
| | | | | tcaccgtggacaagagcaggtggcagcaggggaacgtcttctcatgctccgtgatgcatgaggctctgcacaa |
| | | | | ccactacacgcagaagagcctctccctgtctccgggtaaa |
| 429 | | | Human Heavy Chain Constant | ASTKGPSVFPLAPSSKSTSGGTAALGCLVKDYFPEPVTVSWNSGALTSGVHTFPAVLQSS |
| | | | Region (IGHG1*01) Protein | GLYSLSSVVTVPSSSLGTQTYICNVNHKPSNTKVDKKVEPKSCDKTHTCPPCPAPELLGGP |
| | | | Sequence (P01857) | SVFLFPPKPKDTLMISRTPEVTCVVVDVSHEDPEVKFNWYVDGVEVHNAKTKPREEQYN |
| | | | | STYRVVSVLTVLHQDWLNGKEYKCKVSNKALPAPIEKTISKAKGQPREPQVYTLPPSRDE |
| | | | | LTKNQVSLTCLVKGFYPSDIAVEWESNGQPENNYKTTPPVLDSDGSFFLYSKLTVDKSRW |
| | | | | QQGNVFSCSVMHEALHNHYTQKSLSLSPGK |
| 430 | Human IgG1 | IGHG1*02 or | Human Heavy Chain Constant | gcctccaccaagggcccatcggtcttccccctggcaccctcctccaagagcacctctgggggcacagcggccct |
| | constant | IGHG1*05 | Region (IGHG1*02 or | gggctgcctggtcaaggactacttccccgaaccggtgacggtgtcgtggaactcaggcgccctgaccagcggc |
| | region | | IGHG1*05) Nucleotide | gtgcacaccttcccggctgtcctacagtcctcaggactctactccctcagcagcgtggtgaccgtgccctccagc |
| | | | Sequence | agcttgggcacccagacctacatctgcaacgtgaatcacaagcccagcaacaccaaggtggacaagaaagtt |
| | | | | gagcccaaatcttgtgacaaaactcacacatgcccaccgtgcccagcacctgaactcctggggggaccgtcag |
| | | | | tcttcctcttccccccaaaacccaaggacaccctcatgatctcccggacccctgaggtcacatgcgtggtggtgg |
| | | | | acgtgagccacgaagaccctgaggtcaagttcaactggtacgtggacggcgtggaggtgcataatgccaaga |
| | | | | caaagccgcgggaggagcagtacaacagcacgtaccgtgtggtcagcgtcctcaccgtcctgcaccaggact |
| | | | | ggctgaatggcaaggagtacaagtgcaaggtctccaacaaagccctcccagcccccatcgagaaaaccatct |
| | | | | ccaaagccaaagggcagccccgagaaccacaggtgtacaccctgcccccatcccgggatgagctgaccaaga |
| | | | | accaggtcagcctgacctgcctggtcaaaggcttctatcccagcgacatcgccgtggagtgggagagcaatgg |
| | | | | gcagccggagaacaactacaagaccacgcctcccgtgctggactccgacggctccttcttcctctacagcaagc |
| | | | | tcaccgtggacaagagcaggtggcagcaggggaacgtcttctcatgctccgtgatgcatgaggctctgcacaa |
| | | | | ccactacacgcagaagagcctctccctgtctccgggtaaa |
| 431 | | | Human Heavy Chain Constant | A S T K G P S V F P L A P S S K S T S G G T A A L G C L V K D Y F P E P V T V S W N |
| | | | Region (IGHG1*02) Protein | S G A L T S G V H T F P A V L Q S S G L Y S L S S V V T V P S S S L G T Q T Y I C N V |
| | | | Sequence | N H K P S N T K V D K K V E P K S C D K T H T C P P C P A P E L L G G P S V F L F P |
| | | | | P K P K D T L M I S R T P E V T C V V V D V S H E D P E V K F N W Y V D G V E V H |
| | | | | N A K T K P R E E Q Y N S T Y R V V S V L T V L H Q D W L N G K E Y K C K V S N K |
| | | | | A L P A P I E K T I S K A K G Q P R E P Q V Y T L P P S R D E L T K N Q V S L T C L V |
| | | | | K G F Y P S D I A V E W E S N G Q P E N N Y K T T P P V L D S D G S F F L Y S K L T |
| | | | | V D K S R W Q Q G N V F S C S V M H E A L H N H Y T Q K S L S L S P G K |
| 432 | Human IgG1 | IGHG1*03 | Human Heavy Chain Constant | gcctccaccaagggcccatcggtcttccccctggcaccctcctccaagagcacctctgggggcacagcggccct |
| | constant | | Region (IGHG1*03) Nucleotide | gggctgcctggtcaaggactacttccccgaaccggtgacggtgtcgtggaactcaggcgccctgaccagcggc |
| | region | | Sequence (Y14737) | gtgcacaccttcccggctgtcctacagtcctcaggactctactccctcagcagcgtggtgaccgtgccctccagc |
| | | | | agcttgggcacccagacctacatctgcaacgtgaatcacaagcccagcaacaccaaggtggacaagagagtt |
| | | | | gagcccaaatcttgtgacaaaactcacacatgcccaccgtgcccagcacctgaactcctggggggaccgtcag |
| | | | | tcttcctcttccccccaaaacccaaggacaccctcatgatctcccggacccctgaggtcacatgcgtggtggtgg |
| | | | | acgtgagccacgaagaccctgaggtcaagttcaactggtacgtggacggcgtggaggtgcataatgccaaga |
| | | | | caaagccgcgggaggagcagtacaacagcacgtaccgtgtggtcagcgtcctcaccgtcctgcaccaggact |
| | | | | ggctgaatggcaaggagtacaagtgcaaggtctccaacaaagccctcccagcccccatcgagaaaaccatct |
| | | | | ccaaagccaaagggcagccccgagaaccacaggtgtacaccctgcccccatcccgggaggagatgaccaag |
| | | | | aaccaggtcagcctgacctgcctggtcaaaggcttctatcccagcgacatcgccgtggagtgggagagcaatg |
| | | | | ggcagccggagaacaactacaagaccacgcctcccgtgctggactccgacggctccttcttcctctatagcaag |
| | | | | ctcaccgtggacaagagcaggtggcagcaggggaacgtcttctcatgctccgtgatgcatgaggctctgcaca |
| | | | | accactacacgcagaagagcctctccctgtccccgggtaaa |
| 433 | | | Human Heavy Chain Constant | A S T K G P S V F P L A P S S K S T S G G T A A L G C L V K D Y F P E P V T V S W N |
| | | | Region (IGHG1*03) Protein | S G A L T S G V H T F P A V L Q S S G L Y S L S S V V T V P S S S L G T Q T Y I C N V |
| | | | Sequence | N H K P S N T K V D K R V E P K S C D K T H T C P P C P A P E L L G G P S V F L F P |
| | | | | P K P K D T L M I S R T P E V T C V V V D V S H E D P E V K E N W Y V D G V E V H |
| | | | | N A K T K P R E E Q Y N S T Y R V V S V L T V L H Q D W L N G K E Y K C K V S N K |
| | | | | A L P A P I E K T I S K A K G Q P R E P Q V Y T L P P S R E E M T K N Q V S L T C L V |
| | | | | K G F Y P S D I A V E W E S N G Q P E N N Y K T T P P V L D S D G S F F L Y S K L T |
| | | | | V D K S R W Q Q G N V F S C S V M H E A L H N H Y T Q K S L S L S P G K |
| 434 | Human IgG1 | IGHG1*04 | Human Heavy Chain Constant | gcctccaccaagggcccatcggtcttccccctggcaccctcctccaagagcacctctgggggcacagcggccct |
| | constant | | Region (IGHG1*04) Nucleotide | gggctgcctggtcaaggactacttccccgaaccggtgacggtgtcgtggaactcaggcgccctgaccagcggc |
| | region | | Sequence | gtgcacaccttcccggctgtcctacagtcctcaggactctactccctcagcagcgtggtgaccgtgccctccagc |
| | | | | agcttgggcacccagacctacatctgcaacgtgaatcacaagcccagcaacaccaaggtggacaagaaagtt |
| | | | | gagcccaaatcttgtgacaaaactcacacatgcccaccgtgcccagcacctgaactcctggggggaccgtcag |
| | | | | tcttcctcttccccccaaaacccaaggacaccctcatgatctcccggacccctgaggtcacatgcgtggtggtgg |
| | | | | acgtgagccacgaagaccctgaggtcaagttcaactggtacgtggacggcgtggaggtgcataatgccaaga |
| | | | | caaagccgcgggaggagcagtacaacagcacgtaccgtgtggtcagcgtcctcaccgtcctgcaccaggact |
| | | | | ggctgaatggcaaggagtacaagtgcaaggtctccaacaaagccctcccagcccccatcgagaaaaccatct |
| | | | | ccaaagccaaagggcagccccgagaaccacaggtgtacaccctgcccccatcccgggatgagctgaccaaga |
| | | | | accaggtcagcctgacctgcctggtcaaaggcttctatcccagcgacatcgccgtggagtgggagagcaatgg |
| | | | | gcagccggagaacaactacaagaccacgcctcccgtgctggactccgacggctccttcttcctctacagcaagc |
| | | | | tcaccgtggacaagagcaggtggcagcaggggaacatcttctcatgctccgtgatgcatgaggctctgcacaa |
| | | | | ccactacacgcagaagagcctctccctgtctccgggtaaa |
| 435 | | | Human Heavy Chain Constant | ASTKGPSVFPLAPSSKSTSGGTAALGCLVKDYFPEPVTVSWNSGALTSGVHTFPAVLQSS |
| | | | Region (IGHG1*04) Protein | GLYSLSSVVTVPSSSLGTQTYICNVNHKPSNTKVDKKVEPKSCDKTHTCPPCPAPELLGGP |
| | | | Sequence | SVFLFPPKPKDTLMISRTPEVTCVVVDVSHEDPEVKFNWYVDGVEVHNAKTKPREEQYN |
| | | | | STYRVVSVLTVLHQDWLNGKEYKCKVSNKALPAPIEKTISKAKGQPREPQVYTLPPSRDE |
| | | | | LTKNQVSLTCLVKGFYPSDIAVEWESNGQPENNYKTTPPVLDSDGSFFLYSKLTVDKSRW |
| | | | | QQGNIFSCSVMHEALHNHYTQKSLSLSPGK |
| 436 | Disabled | Disabled | Disabled Human IGHG1*01 | Gcctccaccaagggcccatcggtcttccccctggcaccctcctccaagagcacctctgggggcacagcggccct |
| | Human IgG1 | human | Heavy Chain Constant Region | gggctgcctggtcaaggactacttccccgaaccggtgacggtgtcgtggaactcaggcgccctgaccagcggc |
| | heavy chain | IGHG1*01 | Nucleotide Sequence. | gtgcacaccttcccggctgtcctacagtcctcaggactctactccctcagcagcgtggtgaccgtgccctccagc |
| | constant | | | agcttgggcacccagacctacatctgcaacgtgaatcacaagcccagcaacaccaaggtggacaagaaagtg |
| | region | | | gagcccaaatcttgtgacaaaactcacacatgcccaccgtgcccagcacctgaactcgcgggggcaccgtcag |
| | | | | tcttcctcttccccccaaaacccaaggacaccctcatgatctcccggacccctgaggtcacatgcgtggtggtgg |
| | | | | acgtgagccacgaagaccctgaggtcaagttcaactggtacgtggacggcgtggaggtgcataatgccaaga |
| | | | | caaagccgcgggaggagcagtacaacagcacgtaccgtgtggtcagcgtcctcaccgtcctgcaccaggact |
| | | | | ggctgaatggcaaggagtacaagtgcaaggtctccaacaaagccctcccagcccccatcgagaaaaccatct |
| | | | | ccaaagccaaagggcagccccgagaaccacaggtgtacaccctgcccccatcccgggatgagctgaccaaga |
| | | | | accaggtcagcctgacctgcctggtcaaaggcttctatcccagcgacatcgccgtggagtgggagagcaatgg |
| | | | | gcagccggagaacaactacaagaccacgcctcccgtgctggactccgacggctccttcttcctctacagcaagc |
| | | | | tcaccgtggacaagagcaggtggcagcaggggaacgtcttctcatgctccgtgatgcatgaggctctgcacaa |
| | | | | ccactacacgcagaagagcctctccctgtctccgggtaaa |
| 437 | | | Disabled Human IGHG1*01 | ASTKGPSVFPLAPSSKSTSGGTAALGCLVKDYFPEPVTVSWNSGALTSGVHTFPAVLQSS |
| | | | Heavy Chain Constant Region | GLYSLSSVVTVPSSSLGTQTYICNVNHKPSNTKVDKKVEPKSCDKTHTCPPCPAPELAGA |
| | | | Amino Acid Sequence. Two | PSVFLFPPKPKDTLMISRTPEVTCVVVDVSHEDPEVKFNWYVDGVEVHNAKTKPREEQY |
| | | | residues that differ from the | NSTYRVVSVLTVLHQDWLNGKEYKCKVSNKALPAPIEKTISKAKGQPREPQVYTLPPSRD |
| | | | wild-type sequence are | ELTKNQVSLTCLVKGFYPSDIAVEWESNGQPENNYKTTPPVLDSDGSFFLYSKLTVDKSR |
| | | | identified in bold. | WQQGNVFSCSVMHEALHNHYTQKSLSLSPGK |
| 438 | Human IgG2 | IGHG2*01 or | Human Heavy Chain Constant | gcctccaccaagggcccatcggtcttccccctggcgccctgctccaggagcacctccgagagcacagccgccct |
| | constant | IGHG2*04 or | Region (IGHG2*01 or IGHG2*03 | gggctgcctggtcaaggactacttccccgaaccggtgacggtgtcgtggaactcaggcgctctgaccagcggc |
| | region | IDHG2*5 | or IGHG2*05) Nucleotide | gtgcacaccttcccagctgtcctacagtcctcaggactctactccctcagcagcgtggtgaccgtgccctccagc |
| | | | Sequence | aacttcggcacccagacctacacctgcaacgtagatcacaagcccagcaacaccaaggtggacaagacagtt |
| | | | | gagcgcaaatgttgtgtcgagtgcccaccgtgcccagcaccacctgtggcaggaccgtcttcctcttcccc |
| | | | | ccaaaacccaaggacaccctcatgatctcccggacccctgaggtcacgtgcgtggtggtggacgtgagccacg |
| | | | | aagaccccgaggtccagttcaactggtacgtggacggcgtggaggtgcataatgccaagacaaagccacggg |
| | | | | aggagcagttcaacagcacgttccgtgtggtcagcgtcctcaccgttgtgcaccaggactggctgaacggcaa |
| | | | | ggagtacaagtgcaaggtctccaacaaaggcctcccagcccccatcgagaaaaccatctccaaaaccaaagg |
| | | | | gcagccccgagaaccacaggtgtacaccctgcccccatcccgggaggagatgaccaagaaccaggtcagcct |
| | | | | gacctgcctggtcaaaggcttctaccccagcgacatcgccgtggagtgggagagcaatgggcagccggagaa |
| | | | | caactacaagaccacacctcccatgctggactccgacggctccttcttcctctacagcaagctcaccgtggaca |
| | | | | agagcaggtggcagcaggggaacgtcttctcatgctccgtgatgcatgaggctctgcacaaccactacacgca |
| | | | | gaagagcctctccctgtctccgggtaaa |
| 439 | | | Human Heavy Chain Constant | ASTKGPSVFPLAPCSRSTSESTAALGCLVKDYFPEPVTVSWNSGALTSGVHTFPAVLQSS |
| | | | Region (IGHG2*01) Protein | GLYSLSSVVTVPSSNFGTQTYTCNVDHKPSNTKVDKTVERKCCVECPPCPAPPVAGPSVF |
| | | | Sequence | LFPPKPKDTLMISRTPEVTCVVVDVSHEDPEVQFNWYVDGVEVHNAKTKPREEQFNST |
| | | | | FRVVSVLTVVHQDWLNGKEYKCKVSNKGLPAPIEKTISKTKGQPREPQVYTLPPSREEMT |
| | | | | KNQVSLTCLVKGFYPSDIAVEWESNGQPENNYKTTPPMLDSDGSFFLYSKLTVDKSRW |
| | | | | QQGNVFSCSVMHEALHNHYTQKSLSLSPGK |
| 567 | Human IgG2 | IGHG2*02 | Human Heavy Chain Constant | GCCTCCACCAAGGGCCCATCGGTCTTCCCCCTGGCGCCCTGCTCCAGGAGCACCTCC |
| | constant | | Region (IGHG2*02) Nucleotide | GAGAGCACAGCGGCCCTGGGCTGCCTGGTCAAGGACTACTTCCCCGAACCGGTGAC |
| | region | | Sequence | GGTGTCGTGGAACTCAGGCGCTCTGACCAGCGGCGTGCACACCTTCCCGGCTCCT |
| | | | | ACAGTCCTCAGGACTCTACTCCCTCAGCAGCGTGGTGACCGTGACCTCCAGCAACTT |
| | | | | CGGCACCCAGACCTACACCTGCAACGTAGATCACAAGCCCAGCAACACCAAGGTGG |
| | | | | ACAAGACAGTTGAGCGCAAATGTTGTGTCGAGTGCCCACCGTGCCCAGCACCACCT |
| | | | | GTGGCAGGACCGTCAGTCTTCCTCTTCCCCCCAAAACCCAAGGACACCCTCATGATCT |
| | | | | CCCGGACCCCTGAGGTCACGTGCGTGGTGGTGGACGTGAGCCACGAAGACCCCGA |
| | | | | GGTCCAGTTCAACTGGTACGTGGACGGCATGGAGGTGCATAATGCCAAGACAAAGC |
| | | | | CACGGGAGGAGCAGTTCAACAGCACGTTCCGTGTGGTCAGCGTCCTCACCGTCGTG |
| | | | | CACCAGGACTGGCTGAACGGCAAGGAGTACAAGTGCAAGGTCTCCAACAAAGGCCT |
| | | | | CCCAGCCCCCATCGAGAAAACCATCTCCAAAACCAAAGGGCAGCCCCGAGAACCAC |
| | | | | AGGTGTACACCCTGCCCCCATCCCGGGAGGAGATGACCAAGAACCAGGTCAGCCTG |
| | | | | ACCTGCCTGGTCAAAGGCTTCTACCCCAGCGACATCGCCGTGGAGTGGGAGAGCAA |
| | | | | TGGGCAGCCGGAGAACAACTACAAGACCACACCTCCCATGCTGGACTCCGACGGCT |
| | | | | CCTTCTTCCTCTACAGCAAGCTCACCGTGGACAAGAGCAGGTGGCAGCAGGGGAAC |
| | | | | GTCTTCTCATGCTCCGTGATGCATGAGGCTCTGCACAACCACTACACACAGAAGAGC |
| | | | | CTCTCCCTGTCTCCGGGTAAA |
| 440 | | | Human Heavy Chain Constant | ASTKGPSVFPLAPCSRSTSESTAALGCLVKDYFPEPVTVSWNSGALTSGVHTFPAVLQSS |
| | | | Region (IGHG2*02) Protein | GLYSLSSVVTVTSSNFGTQTYTCNVDHKPSNTKVDKTVERKCCVECPPCPAPPVAGPSVF |
| | | | Sequence | LFPPKPKDTLMISRTPEVTCVVVDVSHEDPEVQFNWYVDGMEVHNAKTKPREEQFNST |
| | | | | FRVVSVLTVVHQDWLNGKEYKCKVSNKGLPAPIEKTISKTKGQPREPQVYTLPPSREEMT |
| | | | | KNQVSLTCLVKGFYPSDIAVEWESNGQPENNYKTTPPMLDSDGSFFLYSKLTVDKSRW |
| | | | | QQGNVFSCSVMHEALHNHYTQKSLSLSPGK |
| 441 | Human IgG2 | IGHG2*04 | Human Heavy Chain Constant | gcctccaccaagggcccatcggtcttccccctggcgccctgctccaggagcacctccgagagcacagcggccct |
| | constant | | Region (IGHG2*04) Nucleotide | gggctgcctggtcaaggactacttccccgaaccggtgacggtgtcgtggaactcaggcgctctgaccagcggc |
| | region | | Sequence | gtgcacaccttcccagctgtcctacagtcctcaggactctactccctcagcagcgtggtgaccgtgccctccagc |
| | | | | agcttgggcacccagacctacacctgcaacgtagatcacaagcccagcaacaccaaggtggacaagacagtt |
| | | | | gagcgcaaatgttgtgtcgagtgcccaccgtgcccagcaccacctgtggcaggaccgtcagtcttcctcttcccc |
| | | | | ccaaaacccaaggacaccctcatgatctcccggacccctgaggtcacgtgcgtggtggtggacgtgagccacg |
| | | | | aagaccccgaggtccagttcaactggtacgtggacggcgtggaggtgcataatgccaagacaaagccacggg |
| | | | | aggagcagttcaacagcacgttccgtgtggtcagcgtcctcaccgttgtgcaccaggactggctgaacggcaa |
| | | | | ggagtacaagtgcaaggtctccaacaaaggcctcccagcccccatcgagaaaaccatctccaaaaccaaagg |
| | | | | gcagccccgagaaccacaggtgtacaccctgcccccatcccgggaggagatgaccaagaaccaggtcagcct |
| | | | | gacctgcctggtcaaaggcttctaccccagcgacatcgccgtggagtgggagagcaatgggcagccggagaa |
| | | | | caactacaagaccacacctcccatgctggactccgacggctccttcttcctctacagcaagctcaccgtggaca |
| | | | | agagcaggtggcagcaggggaacgtcttctcatgctccgtgatgcatgaggctctgcacaaccactacacgca |
| | | | | gaagagcctctccctgtctccgggtaaa |
| 442 | | | Human Heavy Chain Constant | ASTKGPSVFPLAPCSRSTSESTAALGCLVKDYFPEPVTVSWNSGALTSGVHTFPAVLQSS |
| | | | Region (IGHG2*04) Protein | GLYSLSSVVTVPSSSLGTQTYTCNVDHKPSNTKVDKTVERKCCVECPPCPAPPVAGPSVF |
| | | | Sequence | LFPPKPKDTLMISRTPEVTCVVVDVSHEDPEVQFNWYVDGVEVHNAKTKPREEQFNST |
| | | | | FRVVSVLTVVHQDWLNGKEYKCKVSNKGLPAPIEKTISKTKGQPREPQVYTLPPSREEMT |
| | | | | KNQVSLTCLVKGFYPSDIAVEWESNGQPENNYKTTPPMLDSDGSFFLYSKLTVDKSRW |
| | | | | QQGNVFSCSVMHEALHNHYTQKSLSLSPGK |
| 443 | Human IgG2 | IGHG2*06 | Human Heavy Chain Constant | GCCTCCACCAAGGGCCCATCGGTCTTCCCCCTGGCGCCCTGCTCCAGGAGCACCTCC |
| | constant | | Region (IGHG2*06) Nucleotide | GAGAGCACAGCGGCCCTGGGCTGCCTGGTCAAGGACTACTTCCCCGAACCGGTGAC |
| | region | | Sequence | GGTGTCGTGGAACTCAGGCGCTCTGACCAGCGGCGTGCACACCTTCCCGGCTGTCCT |
| | | | | ACAGTCCTCAGGACTCTACTCCCTCAGCAGCGTGGTGACCGTGCCCTCCAGCAACTT |
| | | | | CGGCACCCAGACCTACACCTGCAACGTAGATCACAAGCCCAGCAACACCAAGGTGG |
| | | | | ACAAGACAGTTGAGCGCAAATGTTGTGTCGAGTGCCCACCGTGCCCAGCACCACCT |
| | | | | GTGGCAGGACCGTCAGTCTTCCTCTTCCCCCCAAAACCCAAGGACACCCTCATGATCT |
| | | | | CCCGGACCCCTGAGGTCACGTGCGTGGTGGTGGACGTGAGCCACGAAGACCCCGA |
| | | | | GGTCCAGTTCAACTGGTACGTGGACGGCGTGGAGGTGCATAATGCCAAGACAAAGC |
| | | | | CACGGGAGGAGCAGTTCAACAGCACGTTCCGTGTGGTCAGCGTCCTCACCGTCGTG |
| | | | | CACCAGGACTGGCTGAACGGCAAGGAGTACAAGTGCAAGGTCTCCAACAAAGGCCT |
| | | | | CCCAGCCCCCATCGAGAAAACCATCTCCAAAACCAAAGGGCAGCCCCGAGAACCAC |
| | | | | AGGTGTACACCCTGCCCCCATCCCGGGAGGAGATGACCAAGAACCAGGTCAGCCTG |
| | | | | ACCTGCCTGGTCAAAGGCTTCTACCCCAGCGACATCTCCGTGGAGTGGGAGAGCAA |
| | | | | TGGGCAGCCGGAGAACAACTACAAGACCACACCTCCCATGCTGGACTCCGACGGCT |
| | | | | CCTTCTTCCTCTACAGCAAGCTCACCGTGGACAAGAGCAGGTGGCAGCAGGGGAAC |
| | | | | GTCTTCTCATGCTCCGTGATGCATGAGGCTCTGCACAACCACTACACACAGAAGAGC |
| | | | | CTCTCCCTGTCTCCGGGTAAA |
| 444 | | | Human Heavy Chain Constant | ASTKGPSVFPLAPCSRSTSESTAALGCLVKDYFPEPVTVSWNSGALTSGVHTFPAVLQSS |
| | | | Region (IGHG2*06) Protein | GLYSLSSVVTVPSSNFGTQTYTCNVDHKPSNTKVDKTVERKCCVECPPCPAPPVAGPSVF |
| | | | Sequence | LFPPKPKDTLMISRTPEVTCVVVDVSHEDPEVQFNWYVDGVEVHNAKTKPREEQFNST |
| | | | | FRVVSVLTVVHQDWLNGKEYKCKVSNKGLPAPIEKTISKTKGQPREPQVYTLPPSREEMT |
| | | | | KNQVSLTCLVKGFYPSDISVEWESNGQPENNYKTTPPMLDSDGSFFLYSKLTVDKSRWQ |
| | | | | QGNVFSCSVMHEALHNHYTQKSLSLSPGK |
| 445 | Human IgG4 | IGHG4*01 or | Human Heavy Chain Constant | gcttccaccaagggcccatccgtcttccccctggcgccctgctccaggagcacctccgagagcacagccgccct |
| | constant | IGHG4*04 | Region (IGHG4*01 or | gggctgcctggtcaaggactacttccccgaaccggtgacggtgtcgtggaactcaggcgccctgaccagcggc |
| | region | | IGHG4*04) Nucleotide | gtgcacaccttcccggctgtcctacagtcctcaggactctactccctcagcagcgtggtgaccgtgccctccagc |
| | | | Sequence | agcttgggcacgaagacctacacctgcaacgtagatcacaagcccagcaacaccaaggtggacaagagagtt |
| | | | | gagtccaaatatggtcccccatgcccatcatgcccagcacctgagttcctggggggaccatcagtcttcctgttc |
| | | | | cccccaaaacccaaggacactctcatgatctcccggacccctgaggtcacgtgcgtggtggtggacgtgagcc |
| | | | | aggaagaccccgaggtccagttcaactggtacgtggatggcgtggaggtgcataatgccaagacaaagccgc |
| | | | | gggaggagcagttcaacagcacgtaccgtgtggtcagcgtcctcaccgtcctgcaccaggactggctgaacgg |
| | | | | caaggagtacaagtgcaaggtctccaacaaaggcctcccgtcctccatcgagaaaaccatctccaaagccaa |
| | | | | agggcagccccgagagccacaggtgtacaccctgcccccatcccaggaggagatgaccaagaaccaggtca |
| | | | | gcctgacctgcctggtcaaaggcttctaccccagcgacatcgccgtggagtgggagagcaatgggcagccgga |
| | | | | gaacaactacaagaccacgcctcccgtgctggactccgacggctccttcttcctctacagcaggctaaccgtgg |
| | | | | acaagagcaggtggcaggaggggaatgtcttctcatgctccgtgatgcatgaggctctgcacaaccactacac |
| | | | | acagaagagcctctccctgtctctgggtaaa |
| 446 | | | Human Heavy Chain Constant | ASTKGPSVFPLAPCSRSTSESTAALGCLVKDYFPEPVTVSWNSGALTSGVHTFPAVLQSS |
| | | | Region (IGHG4*01) Protein | GLYSLSSVVTVPSSSLGTKTYTCNVDHKPSNTKVDKRVESKYGPPCPSCPAPEFLGGPSVF |
| | | | Sequence (P01861) | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 447 | Human IgG4 | IGHG4*02 | Human Heavy Chain Constant | gcttccaccaagggcccatccgtcttccccctggcgccctgctccaggagcacctccgagagcacagccgccct |
| | constant | | Region (IGHG4*02) Nucleotide | gggctgcctggtcaaggactacttccccgaaccggtgacggtgtcgtggaactcaggcgccctgaccagcggc |
| | region | | Sequence | gtgcacaccttcccggctgtcctacagtcctcaggactctactccctcagcagcgtggtgaccgtgccctccagc |
| | | | | agcttgggcacgaagacctacacctgcaacgtagatcacaagcccagcaacaccaaggtggacaagagagtt |
| | | | | gagtccaaatatggtcccccgtgcccatcatgcccagcacctgagttcctggggggaccatcagtcttcctgttc |
| | | | | cccccaaaacccaaggacactctcatgatctcccggacccctgaggtcacgtgcgtggtggtggacgtgagcc |
| | | | | aggaagaccccgaggtccagttcaactggtacgtggatggcgtggaggtgcataatgccaagacaaagccgc |
| | | | | gggaggagcagttcaacagcacgtaccgtgtggtcagcgtcctcaccgtcgtgcaccaggactggctgaacgg |
| | | | | caaggagtacaagtgcaaggtctccaacaaaggcctcccgtcctccatcgagaaaaccatctccaaagccaa |
| | | | | agggcagccccgagagccacaggtgtacaccctgcccccatcccaggaggagatgaccaagaaccaggtca |
| | | | | gcctgacctgcctggtcaaaggcttctaccccagcgacatcgccgtggagtgggagagcaatgggcagccgga |
| | | | | gaacaactacaagaccacgcctcccgtgctggactccgacggctccttcttcctctacagcaggctaaccgtgg |
| | | | | acaagagcaggtggcaggaggggaatgtcttctcatgctccgtgatgcatgaggctctgcacaaccactacac |
| | | | | gcagaagagcctctccctgtctctgggtaaa |
| 448 | | | Human Heavy Chain Constant | ASTKGPSVFPLAPCSRSTSESTAALGCLVKDYFPEPVTVSWNSGALTSGVHTFPAVLQSS |
| | | | Region (IGHG4*02) Protein | GLYSLSSVVTVPSSSLGTKTYTCNVDHKPSNTKVDKRVESKYGPPCPSCPAPEFLGGPSVF |
| | | | Sequence | LFPPKPKDTLMISRTPEVTCVVVDVSQEDPEVQFNWYVDGVEVHNAKTKPREEQFNST |
| | | | | YRVVSVLTVVHQDWLNGKEYKCKVSNKGLPSSIEKTISKAKGQPREPQVYTLPPSQEEM |
| | | | | TKNQVSLTCLVKGFYPSDIAVEWESNGQPENNYKTTPPVLDSDGSFFLYSRLTVDKSRW |
| | | | | QEGNVFSCSVMHEALHNHYTQKSLSLSLGK |
| 449 | Human IgG4 | IGHG4*03 | Human Heavy Chain Constant | gcttccaccaagggcccatccgtcttccccctggcgccctgctccaggagcacctccgagagcacagccgccct |
| | constant | | Region (IGHG4*03) Nucleotide | gggctgcctggtcaaggactacttccccgaaccggtgacggtgtcgtggaactcaggcgccctgaccagcggc |
| | region | | Sequence | gtgcacaccttcccggctgtcctacagtcctcaggactctactccctcagcagcgtggtgaccgtgccctccagc |
| | | | | agcttgggcacgaagacctacacctgcaacgtagatcacaagcccagcaacaccaaggtggacaagagagtt |
| | | | | gagtccaaatatggtcccccatgcccatcatgcccagcacctgagttcctggggggaccatcagtcttcctgttc |
| | | | | cccccaaaacccaaggacactctcatgatctcccggacccctgaggtcacgtgcgtggtggtggacgtgagcc |
| | | | | aggaagaccccgaggtccagttcaactggtacgtggatggcgtggaggtgcataatgccaagacaaagccgc |
| | | | | gggaggagcagttcaacagcacgtaccgtgtggtcagcgtcctcaccgtcctgcaccaggactggctgaacgg |
| | | | | caaggagtacaagtgcaaggtctccaacaaaggcctcccgtcctccatcgagaaaaccatctccaaagccaa |
| | | | | agggcagccccgagagccacaggtgtacaccctgcccccatcccaggaggagatgaccaagaaccaggtca |
| | | | | gcctgacctgcctggtcaaaggcttctaccccagcgacatcgccgtggagtgggagagcaatgggcagccgga |
| | | | | gaacaactacaagaccacgcctcccgtgctggactccgacggctccttcttcctctacagcaagctcaccgtgg |
| | | | | acaagagcaggtggcaggaggggaacgtcttctcatgctccgtgatgcatgaggctctgcacaaccactacac |
| | | | | gcagaagagcctctccctgtctctgggtaaa |
| 450 | | | Human Heavy Chain Constant | ASTKGPSVFPLAPCSRSTSESTAALGCLVKDYFPEPVTVSWNSGALTSGVHTFPAVLQSS |
| | | | Region (IGHG4*03) Protein | GLYSLSSVVTVPSSSLGTKTYTCNVDHKPSNTKVDKRVESKYGPPCPSCPAPEFLGGFPSVF |
| | | | Sequence | LFPPKPKDTLMISRTPEVTCVVVDVSQEDPEVQFNWYVDGVEVHNAKTKPREEQFNST |
| | | | | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSKLTVDKSRWQ |
| | | | | EGNVFSCSVMHEALHNHYTQKSLSLSLGK |
| 451 | Human IgG4- | IGHG4-PE | Human Heavy Chain Constant | gcctccaccaagggcccatccgtcttccccctggcgccctgctccaggagcacctccgagagcacggccgccct |
| | PE constant | | Region (IGHG4-PE) Nucleotide | gggctgcctggtcaaggactacttccccgaaccagtgacggtgtcgtggaactcaggcgccctgaccagcggc |
| | region | | Sequence Version A | gtgcacaccttcccggctgtcctacagtcctcaggactctactccctcagcagcgtggtgaccgtgccctccagc |
| | | | | agcttgggcacgaagacctacacctgcaacgtagatcacaagcccagcaacaccaaggtggacaagagagtt |
| | | | | gagtccaaatatggtcccccatgcccaccatgcccagcgcctgaatttgaggggggaccatcagtcttctgttc |
| | | | | cccccaaaacccaaggacactctcatgatctcccggacccctgaggtcacgtgcgtggtggtggacgtgagcc |
| | | | | aggaagaccccgaggtccagttcaactggtacgtggatggcgtggaggtgcataatgccaagacaaagccgc |
| | | | | gggaggagcagttcaacagcacgtaccgtgtggtcagcgtcctcaccgtcctgcaccaggactggctgaacgg |
| | | | | caaggagtacaagtgcaaggtctccaacaaaggcctcccgtcatcgatcgagaaaaccatctccaaagccaa |
| | | | | agggcagccccgagagccacaggtgtacaccctgcccccatcccaggaggagatgaccaagaaccaggtca |
| | | | | gcctgacctgcctggtcaaaggcttctaccccagcgacatcgccgtggagtgggagagcaatgggcagccgga |
| | | | | gaacaactacaagaccacgcctcccgtgctggactccgacggatccttcttcctctacagcaggctaaccgtgg |
| | | | | acaagagcaggtggcaggaggggaatgtcttctcatgctccgtgatgcatgaggctctgcacaaccactacac |
| | | | | acagaagagcctctccctgtctctgggtaaa |
| 452 | | | Human Heavy Chain Constant | gcctccaccaagggacctagcgtgttccctctcgccccctgttccaggtccacaagcgagtccaccgctgccctc |
| | | | Region (IGHG4-PE) Nucleotide | ggctgtctggtgaaagactactttcccgagcccgtgaccgtctcctggaatagcggagccctgacctccggcgt |
| | | | Sequence Version B | gcacacatttcccgccgtgctgcagagcagcggactgtatagcctgagcagcgtggtgaccgtgcccagctcca |
| | | | | gcctcggcaccaaaacctacacctgcaacgtggaccacaagccctccaacaccaaggtggacaagcgggtgg |
| | | | | agagcaagtacggccccccttgccctccttgtcctgcccctgagttcgagggaggaccctccgtgttcctgtttcc |
| | | | | ccccaaacccaaggacaccctgatgatctcccggacacccgaggtgacctgtgtggtcgtggacgtcagccag |
| | | | | gaggaccccgaggtgcagttcaactggtatgtggacggcgtggaggtgcacaatgccaaaaccaagcccagg |
| | | | | gaggagcagttcaattccacctacagggtggtgagcgtgctgaccgtcctgcatcaggattggctgaacggca |
| | | | | aggagtacaagtgcaaggtgtccaacaagggactgcccagctccatcgagaagaccatcagcaaggctaag |
| | | | | ggccagccgagggagccccaggtgtataccctgcctcctagccaggaagagatgaccaagaaccaagtgtcc |
| | | | | ctgacctgcctggtgaagggattctacccctccgacatcgccgtggagtgggagagcaatggccagcccgaga |
| | | | | acaactacaaaacaacccctcccgtgctcgatagcgacggcagcttctttctctacagccggctgacagtggac |
| | | | | aagagcaggtggcaggagggcaacgtgttctcctgttccgtgatgcacgaggccctgcacaatcactacaccc |
| | | | | agaagagcctctccctgtccctgggcaag |
| 453 | | | Human Heavy Chain Constant | gccagcaccaagggcccttccgtgttccccctggccccttgcagcaggagcacctccgaatccacagctgccct |
| | | | Region (IGHG4-PE) Nucleotide | gggctgtctggtgaaggactactttcccgagcccgtgaccgtgagctggaacagcggcgctctgacatccggcg |
| | | | Sequence Version C | tccacacctttcctgccgtcctgcagtcctccggcctctactccctgtcctccgtggtgaccgtgcctagctcctcc |
| | | | | ctcggcaccaagacctacacctgtaacgtggaccacaaaccctccaacaccaaggtggacaaacgggtcgag |
| | | | | agcaagtacggccctccctgccctccttgtcctgcccccgagttcgaaggcggacccagcgtgttcctgttccct |
| | | | | cctaagcccaaggacaccctcatgatcagccggacacccgaggtgacctgcgtggtggtggatgtgagccagg |
| | | | | aggaccctgaggtccagttcaactggtatgtggatggcgtggaggtgcacaacgccaagacaaagccccggg |
| | | | | aagagcagttcaactccacctacagggtggtcagcgtgctgaccgtgctgcatcaggactggctgaacggcaa |
| | | | | ggagtacaagtgcaaggtcagcaataagggactgcccagcagcatcgagaagaccatctccaaggctaaag |
| | | | | gccagccccgggaacctcaggtgtacaccctgcctcccagccaggaggagatgaccaagaaccaggtgagcc |
| | | | | tgacctgcctggtgaagggattctacccttccgacatcgccgtggagtgggagtccaacggccagcccgagaa |
| | | | | caattataagaccacccctcccgtcctcgacagcgacggatccttctttctgtactccaggctgaccgtgataa |
| | | | | gtccaggtggcaggaaggcaacgtgttcagctgctccgtgatgcacgaggccctgcacaatcactacaccag |
| | | | | aagtccctgagcctgtccctgggaaag |
| 454 | | | Human Heavy Chain Constant | ASTKGPSVFPLAPCSRSTSESTAALGCLVKDYFPEPVTVSWNSGALTSGVHTFPAVLQSS |
| | | | Region (IGHG4-PE) Protein | GLYSLSSVVTVPSSSLGTKTYTCNVDHKPSNTKVDKRVESKYGPPCPPCPAPEFEGGPSVF |
| | | | Sequence (Amino acid | LFPPKPKDTLMISRTPEVTCVVVDVSQEDPEVQFNWYVDGVEVHNAKTKPREEQFNST |
| | | | substitution shown in BOLD) | YRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQ |
| | | | | EGNVFSCSVMHEALHNHYTQKSLSLSLGK |
| 455 | Inactivated | Inactivated | Inactived Human Heavy Chain | gcctccaccaagggcccatccgtcttccccctggcgccctgctccaggagcacctccgagagcacggccgccct |
| | Human IgG4 | IGHG4 | Constant Region (IGHG4) | gggctgcctggtcaaggactacttccccgaaccagtgacggtgtcgtggaactcaggcgccctgaccagcggc |
| | constant | | Nucleotide Sequence | gtgcacaccttcccggctgtcctacagtcctcaggactctactccctcagcagcgtggtgaccgtgccctccagc |
| | region | | | agcttgggcacgaagacctacacctgcaacgtagatcacaagcccagcaacaccaaggtggacaagagagtt |
| | | | | gagtccaaatatggtcccccatgcccaccatgcccagcgcctccagttgcggggggaccatcagtcttctgttc |
| | | | | cccccaaaacccaaggacactctcatgatctcccggacccctgaggtcacgtgcgtggtggtggacgtgagcc |
| | | | | aggaagaccccgaggtccagttcaactggtacgtggatggcgtggaggtgcataatgccaagacaaagccgc |
| | | | | gggaggagcagttcaacagcacgtaccgtgtggtcagcgtcctcaccgtcctgcaccaggactggctgaacgg |
| | | | | caaggagtacaagtgcaaggtctccaacaaaggcctcccgtcatcgatcgagaaaaccatctccaaagccaa |
| | | | | agggcagccccgagagccacaggtgtacaccctgcccccatcccaggaggagatgaccaagaaccaggtca |
| | | | | gcctgacctgcctggtcaaaggcttctaccccagcgacatcgccgtggagtgggagagcaatgggcagccgga |
| | | | | gaacaactacaagaccacgcctcccgtgctggactccgacggatccttcttcctctacagcaggctaacgtgg |
| | | | | acaagagcaggtggcaggaggggaatgtcttctcatgctccgtgatgcatgaggctctgcacaaccactacac |
| | | | | acagaagagcctctccctgtctctgggtaaa |
| 456 | | | Inactived Human Heavy Chain | ASTKGPSVFPLAPCSRSTSESTAALGCLVKDYFPEPVTVSWNSGALTSGVHTFPAVLQSS |
| | | | Constant Region (IGHG4) | GLYSLSSVVTVPSSSLGTKTYTCNVDHKPSNTKVDKRVESKYGPPCPPCPAPPVAGGPSV |
| | | | Protein Sequence (inactivating | FLFPPKPKDTLMISRTPEVTCVVVDVSQEDPEVQFNWYVDGVEVHNAKTKPREEQFNS |
| | | | mutations from human IgG4 | TYRVVSVLTVLHQDWLNGKEYKCKVSNKGLPSSIEKTISKAKGQPREPQVYTLPPSQEEM |
| | | | shown in bold) | TKNQVSLTCLVKGFYPSDIAVEWESNGQPENNYKTTPPVLDSDGSFFLYSRLTVDKSRW |
| | | | | QEGNVFSCSVMHEALHNHYTQKSLSLSLGK |
| 457 | Human Cκ | IGKC*01 | Human Cκ Light Chain Constant | cgtacggtggccgctccctccgtgttcatcttcccaccttccgacgagcagctgaagtccggcaccgcttctgtcg |
| | constant | | Region (IGKC*01) Nucleotide | tgtgcctgctgaacaacttctacccccgcgaggccaaggtgcagtggaaggtggacaacgccctgcagtccgg |
| | region | | Sequence | caactcccaggaatccgtgaccgagcaggactccaaggacagcacctactccctgtcctccaccctgaccctgt |
| | | | | ccaaggccgactacgagaagcacaaggtgtacgcctgcgaagtgacccaccagggcctgtctagccccgtga |
| | | | | ccaagtctttcaaccggggcgagtgt |
| 458 | | | Cκ Light Chain Constant Region | RTVAAPSVFIFPPSDEQLKSGTASVVCLLNNFYPREAKVQWKVDNALQSGNSQESVTEQ |
| | | | (IGKC*01) Amino Acid Sequence | DSKDSTYSLSSTLTLSKADYEKHKVYACEVTHQGLSSPVTKSFNRGEC |
| 459 | Human Cκ | IGKC*02 | Cκ Light Chain Constant Region | cgaactgtggctgcaccatctgtcttcatcttcccgccatctgatgagcagttgaaatctggaactgcctctgttg |
| | constant | | (IGKC*02) Nucleotide Sequence | tgtgcctgctgaataacttctatcccagagaggccaaagtacagtggaaggtggataacgccctccaatcgggt |
| | region | | | aactcccaggagagtgtcacagagcaggagagcaaggacagcacctacagcctcagcagcaccctgacgct |
| | | | | gagcaaagcagactacgagaaacacaaagtctacgccggcgaagtcacccatcagggcctgagctcgcccgt |
| | | | | cacaaagagcttcaacaggggagagtgt |
| 460 | | | Cκ Light Chain Constant Region | RTVAAPSVFIFPPSDEQLKSGTASVVCLLNNFYPREAKVQWKVDNALQSGNSQESVTEQ |
| | | | (IGKC*02) Amino Acid Sequence | ESKDSTYSLSSTLTLSKADYEKHKVYAGEVTHQGLSSPVTKSFNRGEC |
| 461 | Human Cκ | IGKC*03 | Cκ Light Chain Constant Region | cgaactgtggctgcaccatctgtcttcatcttcccgccatctgatgagcagttgaaatctggaactgcctctgttg |
| | constant | | (IGKC*03) Nucleotide Sequence | tgtgcctgctgaataacttctatcccagagaggccaaagtacagcggaaggtggataacgccctccaatcggg |
| | region | | | taactcccaggagagtgtcacagagcaggagagcaaggacagcacctacagcctcagcagcaccctgacgct |
| | | | | gagcaaagcagactacgagaaacacaaagtctacgcctgcgaagtcacccatcagggcctgagctcgcccgt |
| | | | | cacaaagagcttcaacaggggagagtgt |
| 462 | | | Cκ Light Chain Constant Region | RTVAAPSVFIFPPSDEQLKSGTASVVCLLNNFYPREAKVQRKVDNALQSGNSQESVTEQ |
| | | | (IGKC*03) Amino Acid Sequence | ESKDSTYSLSSTLTLSKADYEKHKVYACEVTHQGLSSPVTKSFNRGEC |
| 463 | Human Cκ | IGKC*04 | Cκ Light Chain Constant Region | cgaactgtggctgcaccatctgtcttcatcttcccgccatctgatgagcagttgaaatctggaactgcctctgttg |
| | constant | | (IGKC*04) Nucleotide Sequence | tgtgcctgctgaataacttctatcccagagaggccaaagtacagtggaaggtggataacgccctccaatcgggt |
| | region | | | aactcccaggagagtgtcacagagcaggacagcaaggacagcacctacagcctcagcagcaccctgacgctg |
| | | | | agcaaagcagactacgagaaacacaaactctacgcctgcgaagtcacccatcagggcctgagctcgccgtc |
| | | | | acaaagagcttcaacaggggagagtgt |
| 464 | | | Cκ Light Chain Constant Region | RTVAAPSVFIFPPSDEQLKSGTASVVCLLNNFYPREAKVQWKVDNALQSGNSQESVTEQ |
| | | | (IGKC*04) Amino Acid Sequence | DSKDSTYSLSSTLTLSKADYEKHKLYACEVTHQGLSSPVTKSFNRGEC |
| 465 | Human Cκ | IGKC*05 | Cκ Light Chain Constant Region | cgaactgtggctgcaccatctgtcttcatcttcccgccatctgatgagcagttgaaatctggaactgcctctgttg |
| | constant | | (IGKC*05) Nucleotide Sequence | tgtgcctgctgaataacttctatcccagagaggccaaagtacagtggaaggtggataacgccctccaatcgggt |
| | region | | | aactcccaggagagtgtcacagagcaggacagcaaggacagcacctacagcctcagcaacaccctgacgctg |
| | | | | agcaaagcagactacgagaaacacaaagtctacgcctgcgaagtcacccatcagggcctgagctcgcccgtc |
| | | | | acaaagagcttcaacaggggagagtgc |
| 466 | | | Cκ Light Chain Constant Region | RTVAAPSVFIFPPSDEQLKSGTASVVCLLNNFYPREAKVQWKVDNALQSGNSQESVTEQ |
| | | | (IGKC*05) Amino Acid Sequence | DSKDSTYSLSNTLTLSKADYEKHKVYACEVTHQGLSSPVTKSFNRGEC |
| 467 | Human Cλ | IGLC1*01 | Cλ lIght Chain Constant Region | cccaaggccaaccccacggtcactctgttcccgccctcctctgaggagctccaagccaacaaggccacatagt |
| | constant | | (IGLC1*01) Nucleotide | gtgtctgatcagtgacttctacccgggagctgtgacagtggcttggaaggcagatggcagccccgtcaaggcg |
| | region | | Sequence | ggagtggagacgaccaaaccctccaaacagagcaacaacaagtacgcggccagcagctacctgagcctgac |
| | | | (ENST00000390321.2) | gcccgagcagtggaagtcccacagaagctacagctgccaggtcacgcatgaagggagcaccgtggagaaga |
| | | | | cagtggcccctacagaatgttca |
| 468 | | | Cλ Light Chain Constant Region | PKANPTVTLFPPSSEELQANKATLVCLISDFYPGAVTVAWKADGSPVKAGVETTKPSKQS |
| | | | (IGLC1*01) Amino Acid Sequence | NNKYAASSYLSLTPEQWKSHRSYSCQVTHEGSTVEKTVAPTECS |
| | | | Sequence (A0A075B6K8) | |
| 469 | Human Cλ | IGLC1*02 | Cλ Light Chain Constant Region | ggtcagcccaaggccaaccccactgtcactctgttcccgccctcctctgaggagctccaagccaacaaggccac |
| | constant | | (IGLC1*02) Nucleotide | actagtgtgtctgatcagtgacttctacccgggagctgtgacagtggcctggaaggcagatggcagccccgtca |
| | region | | Sequence Version A | aggcgggagtggagaccaccaaaccctccaaacagagcaacaacaagtacgcggccagcagctacctgagc |
| | | | | ctgacgcccgagcagtggaagtcccacagaagctacagctgccaggtcacgcatgaagggagcaccgtggag |
| | | | | aagacagtggcccctacagaatgttca |
| 470 | | | Cλ Light Chain Constant Region | ggtcagcccaaggccaaccccactgtcactctgttcccgccctcctctgaggagctccaagccaacaaggccac |
| | | | (IGLC1*02) Nucleotide | actagtgtgtctgatcagtgacttctacccgggagctgtgacagtggcctggaaggcagatggcagccccgtca |
| | | | Sequence Version B | aggcgggagtggagaccaccaaaccctccaaacagagcaacaacaagtacgcggccagcagctacctgagc |
| | | | | ctgacgcccgagcagtggaagtcccacagaagctacagctgccaggtcacgcatgaagggagcaccgtggag |
| | | | | aagacagtggcccctacagaatgttca |
| 471 | | | Cλ Light Chain Constant Region | GQPKANPTVTLFPPSSEELQANKATLVCLISDFYPGAVTVAWKADGSPVKAGVETTKPS |
| | | | (IGLC1*02) Amino Acid | KQSNNKYAASSYLSLTPEQWKSHRSYSCQVTHEGSTVEKTVAPTECS |
| | | | Sequence | |
| 472 | Human Cλ | IGLC1*01 | Cλ Light Chain Constant Region | ggccagcctaaggccgctccttctgtgaccctgttccccccatcctccgaggaactgcaggctaacaaggccac |
| | constant | | (IGLC2*01) Nucleotide | cctcgtgtgcctgatcagcgacttctaccctggcgccgtgaccgtggcctggaaggctgatagctctcctgtgaa |
| | region | | Sequence Version A | ggccggcgtggaaaccaccaccccttccaagcagtccaacaacaaatacgccgcctcctcctacctgtccctga |
| | | | | cccctgagcagtggaagtcccaccggtcctacagctgccaagtgacccacgagggctccaccgtggaaaaga |
| | | | | ccgtggctcctaccgagtgctcc |
| 473 | | | Cλ Light Chain Constant Region | ggccagcctaaagctgcccccagcgtcaccctgtttcctccctccagcgaggagctccaggccaacaaggcca |
| | | | (IGLC2*01) Nucleotide | ccctcgtgtgcctgatctccgacttctatcccggcgctgtgaccgtggcttggaaagccgactccagccctgtca |
| | | | Sequence Version B | aagccggcgtggagaccaccacaccctccaagcagtccaacaacaagtacgccgcctccagctatctctccct |
| | | | | gacccctgagcagtggaagtcccaccggtcctactcctgtcaggtgacccacgagggctccaccgtggaaaag |
| | | | | accgtcgcccccaccgagtgctcc |
| 474 | | | Cλ Light Chain Constant Region | GQPKAAPSVTLFPPSSEELQANKATLVCLISDFYPGAVTVAWKADSSPVKAGVETTTPSK |
| | | | (IGLC1*02) Amino Acid | QSNNKYAASSYLSLTPEQWKSHRSYSCQVTHEGSTVEKTVAPTECS |
| | | | Sequence | |
| 475 | Human Cλ | IGLC2*02 or | Cλ Light Chain Constant Region | ggtcagcccaaggctgccccctcggtcactctgttcccgccctcctctgaggagcttcaagccaacaaggccac |
| | constant | IGLC2*03 | (IGLC2*02 or IGLC2*03) | actggtgtgtctcataagtgacttctacccgggagccgtgacagtggcctggaaggcagatagcagcccgtca |
| | region | | Nucleotide Sequence | aggcgggagtggagaccaccacaccctccaaacaaagcaacaacaagtacgcggccagcagctatctgagc |
| | | | | ctgacgcctgagcagtggaagtcccacagaagctacagctgccaggtcacgcatgaagggagcaccgtggag |
| | | | | aagacagtggcccctacagaatgttca |
| 476 | | | Cλ Light Chain Constant Region | GQPKAAPSVTLFPPSSEELQANKATLVCLISDFYPGAVTVAWKADSSPVKAGVETTTPSK |
| | | | (IGLC2*02) Amino Acid | QSNNKYAASSYLSLTPEQWKSHRSYSCQVTHEGSTVEKTVAPTECS |
| | | | Sequence | |
| 477 | Human Cλ | IGL3*01 | Cλ Light Chain Constant Region | cccaaggctgccccctcggtcactctgttcccaccctcctctgaggagcttcaagccaacaaggccacactggt |
| | constant | | (IGLC3*01) Nucleotide | gtgtctcataagtgacttctacccgggagccgtgacagttgcctggaaggcagatagcagccccgtcaaggcg |
| | region | | Sequence | ggggtggagaccaccacaccctccaaacaaagcaacaacaagtacgcggccagcagctacctgagcctgac |
| | | | | gcctgagcagtggaagtcccacaaaagctacagctgccaggtcacgcatgaagggagcaccgtggagaaga |
| | | | | cagttgcccctacggaatgttca |
| 478 | | | Cλ Light Chain Constant Region | PKAAPSVTLFPPSSEELQANKATLVCLISDFYPGAVTVAWKADSSPVKAGVETTTPSSKQS |
| | | | (IGLC3*01) Amino Acid | NNKYAASSYLSLTPEQWKSHKSYSCQVTHEGSTVEKTVAPTECS |
| | | | Sequence | |
| 479 | Human Cλ | IGLC3*02 | Cλ Light Chain Constant Region | ggtcagcccaaggctgccccctcggtcactctgttcccaccctcctctgaggagcttcaagccaacaaggccac |
| | constant | | (IGLC3*02) Nucleotide | actggtgtgtctcataagtgacttctacccggggccagtgacagttgcctggaaggcagatagcagccccgtca |
| | region | | Sequence | aggcgggggtggagaccaccacaccctccaaacaaagcaacaacaagtacgcggccagcagctacctgagc |
| | | | | ctgacgcctgagcagtggaagtcccacaaaagctacagctgccaggtcacgcatgaagggagcaccgtggag |
| 480 | | | | aagacagtggcccctacggaatgttca |
| | | | Cλ Light Chain Constant Region | GQPKAAPSVTLFPPSSEELQANKATLVCLISDFYPGPVTVAWKADSSPVKAGVETTTPSK |
| | | | (IGLC1*02) Amino Acid | QSNNKYAASSYLSLTPEQWKSHKSYSCQVTHEGSTVEKTVAPTECS |
| | | | Sequence | |
| 481 | Human Cλ | IGLC3*03 | Cλ Light Chain Constant Region | ggtcagcccaaggctgccccctcggtcactctgttcccaccctcctctgaggagcttcaagccaacaaggccac |
| | constant | | (IGLC3*03) Nucleotide | actggtgtgtctcataagtgacttctacccgggagccgtgacagtggcctggaaggcagatagcagcccgtca |
| | region | | Sequence | aggcgggagtggagaccaccacaccctccaaacaaagcaacaacaagtacgcggccagcagctacctgagc |
| | | | | ctgacgcctgagcagtggaagtcccacaaaagctacagctgccaggtcacgcatgaagggagcaccgtggag |
| | | | | aagacagtggcccctacagaatgttca |
| 482 | | | Cλ Light Chain Constant Region | GQPKAAPSVTLFPPSSEELQANKATLVCLISDFYPGAVTVAWKADSSPVKAGVETTTPSK |
| | | | (IGLC3*03) Amino Acid | QSNNKYAASSYLSLTPEQWKSHKSYSCQVTHEGSTVEKTVAPTECS |
| | | | Sequence | |
| 483 | Human Cλ | IGLC3*04 | Cλ Light Chain Constant Region | ggtcagcccaaggctgccccctcggtcactctgttcccgccctcctctgaggagcttcaagccaacaaggccac |
| | constant | | (IGLC3*04) Nucleotide | actggtgtgtctcataagtgacttctacccgggagccgtgacagtggcctggaaggcagatagcagccccgtca |
| | region | | Sequence | aggcgggagtggagaccaccacaccctccaaacaaagcaacaacaagtacgcggccagcagctacctgagc |
| | | | | ctgacgcctgagcagtggaagtcccacagaagctacagctgccaggtcacgcatgaagggagcaccgtggag |
| | | | | aagacagtggcccctacagaatgttca |
| 484 | | | Cλ Light Chain Constant Region | GQPKAAPSVTLFPPSSEELQANKATLVCLISDFYPGAVTVAWKADSSPVKAGVETTTPSK |
| | | | (IGLC3*04) Amino Acid | QSNNKYAASSYLSLTPEQWKSHRSYSCQVTHEGSTVEKTVAPTECS |
| | | | Sequence | |
| 485 | Human Cλ | IGLC6*01 | Cλ Light Chain Constant Region | ggtcagcccaaggctgccccatcggtcactctgttcccgccctcctctgaggagcttcaagccaacaaggccac |
| | constant | | (IGLC6*01) Nucleotide | actggtgtgcctgatcagtgacttctacccgggagctgtgaaagtggcctggaaggcagatggcagccccgtc |
| | region | | Sequence | aacacgggagtggagaccaccacaccctccaaacagagcaacaacaagtacgcggccagcagctacctgag |
| | | | | cctgacgcctgagcagtggaagtcccacagaagctacagctgccaggtcacgcatgaagggagcaccgtgga |
| | | | | gaagacagtggcccctgcagaatgttca |
| 486 | | | Cλ Light Chain Constant Region | GQPKAAPSVTLFPPSSEELQANKATLVCLISDFYPGAVKVAWKADGSPVNTGVETTTPSK |
| | | | (IGLC6*01) Amino Acid | QSNNKYAASSYLSLTPEQWKSHRSYSCQVTHEGSTVEKTVAPAECS |
| | | | Sequence | |
| 487 | Human Cλ | IGLC7*01 or | Cλ Light Chain Constant Region | ggtcagcccaaggctgccccatcggtcactctgttcccaccctcctctgaggagcttcaagccaacaaggccac |
| | constant | IGLC7*02 | (IGLC7*01 or IGLC7*02) | actggtgtgtctcgtaagtgacttctacccgggagccgtgacagtggcctggaaggcagatggcagccccgtca |
| | region | | Nucleotide Sequence | aggtgggagtggagaccaccaaaccctccaaacaaagcaacaacaagtatgcggccagcagctacctgagc |
| | | | | ctgacgcccgagcagtggaagtcccacagaagctacagctgccgggtcacgcatgaagggagcaccgtggag |
| | | | | aagacagtggcccctgcagaatgctct |
| 488 | | | Cλ Light Chain Constant Region | GQPKAAPSVTLFPPSSEELQANKATLVCLVSDFYPGAVTVAWKADGSPVKVGVETTKPS |
| | | | (IGLC7*01) Amino Acid | KQSNNKYAASSYLSLTPEQWKSHRSYSCRVTHEGSTVEKTVAPAECS |
| | | | Sequence | |
| 489 | Human Cλ | IGLC7*03 | Cλ Light Chain Constant Region | GGTCAGCCCAAGGCTGCCCCCTCGGTCACTCTGTTCCCACCCTCCTCTGAGGAGCTTC |
| | constant | | (IGLC7*03) Nucleotide | AAGCCAACAAGGCCACACTGGTGTGTCTCGTAAGTGACTTCAACCCGGGAGCCCGTG |
| | region | | Sequence | ACAGTGGCCTGGAAGGCAGATGGCAGCCCCGTCAAGGTGGGAGTGGAGACCACCA |
| | | | | AACCCTCCAAACAAAGCAACAACAAGTATGCGGCCAGCAGCTACCTGAGCCTGACG |
| | | | | CCCGAGCAGTGGAAGTCCCACAGAAGCTACAGCTGCCGGGTCACGCATGAAGGGA |
| | | | | GCACCGTGGAGAAGACAGTGGCCCCTGCAGAATGCTCT |
| 490 | | | Cλ Light Chain Constant Region | GQPKAAPSVTLFPPSSEELQANKATLVCLVSDFNPGAVTVAWKADGSPVKVGVETTKPS |
| | | | (IGLC7*03) Amino Acid | KQSNNKYAASSYLSLTPEQWKSHRSYSCRVTHEGSTVEKTVAPAECS |
| | | | Sequence | |
| 491 | Rat BMP6 | | Amino acid sequence Uniprot ID | MPGLGRRAQWLCWWWGLLCSCGPPPLRPPLPVAAAAAGGQLLGAGGSPVRAEQPP |
| | | | number: Q04906 | PQSSSSGFLYRRLKTHEKREMQKEILSVLGLPHRPRPLHGLQQPQSPVLPQQQQSQQTA |
| | | | (leader sequence in italics; pro- | REEPPPGRLKSAPLFMLDLYNSLSKDDEEDGVSEGEGLEPESHGRASSSQLKQPSPGAAH |
| | | | peptide underlined; mature | SLNRKSLLAPGPGGSASPLTSAQDSAFLNDADMVMSFVNLVEYDKEFSPRQRHHKEFKF |
| | | | protein bold) | NLSQIPEGEAVTAAEFRVYKDCVVGSFKNQTFLISIYQVLQEHQHRDSDLFLLDTRVVWA |
| | | | | SEEGWLEFDITATSNLWVVTPQHNMGLQLSVVTRDGLHINPRAAGLVGRDGPYDKQP |
| | | | | FMVAFFKVSEVHVRTTRSASSRRRQQSRNRSTQSQDVSRGSSASDYNSSELKTACKKH |
| | | | | ELYVSFQDLGWQDWIIAPKGYAANYCDGECSFPLNAHMNATNHAIVQTLVHLMNP |
| | | | | EYVPKPCCAPTKLNAISVLYFDDNSNVILKKYRNMVVRACGCH |
| 492 | Cynomolgus | | Amino acid sequence Uniprot ID | MPGLGRRAQWLCWWWGLLCSCCGPPRPPLPAAAAAAAGGQLLGDGGSPGRTEQPP |
| | monkey | | number: | PSPQSSSGFLYRRLKTHEKREMQKEILSVLGLPHRPRPLHGLQQPQPPALPQQQQQQQ |
| | BMP6 | | (leader sequence in italics; pro- | QPPRGEPPPGRLKSAPLFMLDLYNALSADDEEDGASEGERQQPWPHEGASSSQPRQP |
| | | | peptide underlined; mature | APGAAHPLNRKSLLAPGPGSGGASPLTSAQDSAFLNDADMVMSFVNLVEYDKEFSPRQ |
| | | | protein bold) | RHHKEFKFNLSQIPEGEAVTAAEFRIYKDCVMGSFKNQTFLISIYQVLQEHQHRDSDLFLL |
| | | | | DTRVVWASEEGWLEFDITATSNLWVVTPQHNMGLQLSVVTRDGVHIHPRAAGLVGR |
| | | | | DGPYDKQPFMVAFFKVSEVHVRTTRSASGRRRQQSRNRSTQSQDVARVSSASDYNSS |
| | | | | ELKTACRKHELYVSFQDLGWQDWIIAPKGYAANYCDGECSFPLNAHMNATNHAIVQ |
| | | | | TLVHLMNPEYVPKPCCAPTKLNAISVLYFDDNSNVILKKYRNMVVRACGCH |
| 493 | Human | Recombinant | Amino acid sequence R&D | QAKHKQRKRLKSSCKRHPLYVDFSDVGWNDWIVAPPGYHAFYCHGECPFPLADHLNST |
| | BMP2 | protein | Systems | NHAIVQTLVNSVNSKIPKACCVPTELSAISMLYLDENEKVVLKNYQDMVVEGCGCR |
| | | | Catalog Number: 355-BM | |
| 494 | Human | Recombinant | Amino acid sequence R&D | SPKHHSQRARKKNKNCRRHSLYVDFSDVGWNDWIVAPPGYQAFYCHGDCPFPLADHL |
| | BMP4 | protein | Systems | NSTNHAIVQTLVNSVNSSIPKACCVPTELSAISMLYLDEYDKVVLKNYQEMVVEGCGCR |
| | | | Catalog Number: 314-BP | |
| 495 | Human | Recombinant | Amino acid sequence R&D | AANKRKNQNRNKSSSHQDSSRMSSVGDYNTSEQKQACKKHELYVSFRDLGWQDWIIA |
| | BMP5 | protein | Systems | PEGYAAFYCDGECSFPLNAHMNATNHAIVQTLVHLMFPDHVPKPCCAPTKLNAISVLYF |
| | | | Catalog Number: 615-BMC | DDSSNVILKKYRNMVVRSCGCH |
| 496 | Human | Recombinant | Amino acid sequence R&D | STGSKQRSQNRSKTPKNQEALRMANVAENSSSDQRQACKKHELYVSFRDLGWQDWII |
| | BMP7 | protein | Systems | APEGYAAYYCEGECAFPLNSYMNATNHAIVQTLVHFINPETVPKPCCAPTQLNAISVLYF |
| | | | Catalog Number: 354-BP | DDSSNVILKKYRNMVVRACGCH |
| 497 | Human | Recombinant | Amino acid sequence Peprotech | SAGAGSHCQKTSLRVNFEDIGWDSWIIAPKEYEAYECKGGCFFPLADDVTPTKHAIVQTL |
| | BMP9 | protein | GDF-2/BMP9 | VHLKFPTKVGKACCVPTKLSPISVLYKDDMGVPTLKYHYEGMSVAECGCR |
| | | | Catalog Number: 120-7 | |
| 498 | CL-66833 | Heavy chain | Nucleotide sequence of heavy | CAGGTCCAGTTTGTGCAGTCTGGGGCTGAGGTGAAGAGTCCTGGGGCCTCTGTGAA |
| | | | chain | GGTTTCCTGCAAGGCTTCTGGATACACCTTCACAAATCATGCTATACATTGGGTGCG |
| | | | | CCAGGCCCCCGGACAAAGGCTTGAGTGGATGGGATGGATCAACGCTGGCAATGGT |
| | | | | AAAACTGATTATTCACAGAACTTCCAGGGCAGAGTCATCATTACCAGGGACACATCC |
| | | | | GCGAACACAGCCTACATGTCCCTGAGCAGCCTGACATCTGAGGACACGGCTTTTTAT |
| | | | | TACTGTACTAGAAGGGTCTACGGTGAATCGTATGACCACTGGGGCCAGGGAACCCT |
| | | | | GGTCACCGTCTCCTCAGCCAGCACCAAGGGCCCTTCCGTGTTCCCCCTGGCCCCTGC |
| | | | | AGCAGGAGCACCTCCGAATCCACAGCTGCCCTGGGCTGTCTGGTGAAGGACTACTTT |
| | | | | CCCGAGCCCGTGACCGTGAGCTGGAACAGCGGCGCTCTGACATCCGGCGTCCACAC |
| | | | | CTTTCCTGCCGTCCTGCAGTCCTCCGGCCTCTACTCCCTGTCCTCCGTGGTGACCGTG |
| | | | | CCTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 499 | CL-66833 | Light chain | Nucleotide sequence of light | GACATCCAGATGACCCAGTCTCCATCCTCCCTGTCTGCATCTGTAGGAGACAGAGTC |
| | | | chain | ACCATCACTTGCCGGGCAAGTCAGGGCATTAGAAATAATTTAGGCTGGTATCAGCA |
| | | | | GAAACCAGGGAAAGCCCCTAAGCGCCTGATCTATGCTGCATCCAGTTTGCAAAGTG |
| | | | | GGGTCCCATCAAGGTTTAGCGGCAGTGGATCTGGGACAGAATTCACTCTCACAATCA |
| | | | | GCAGCCTGCAGCCTGAAGATGTTGCAATTTATTTCTGTCTACAACATCAAATTTACCC |
| | | | | GTGGACGTTCGGCCAAGGGACCAAGGTGGAAACCAAACGTACGGTGGCCGCTCCCT |
| | | | | CCGTGTTCATCTTCCCACCTTCCGACGAGCAGCTGAAGTCCGGCACCGCTTCTGTCGT |
| | | | | GTGCCTGCTGAACAACTTCTACCCCCGCGAGGCCAAGGTGCAGTGGAAGGTGGACA |
| | | | | ACGCCCTGCAGTCCGGCAACTCCCAGGAATCCGTGACCGAGCAGGACTCCAAGGAC |
| | | | | AGCACCTACTCCCTGTCCTCCACCCTGACCCTGTCCAAGGCCGACTACGAGAAGCAC |
| | | | | AAGGTGTACGCCTGCGAAGTGACCCACCAGGGCCTGTCTAGCCCCGTGACCAAGTC |
| | | | | TTTCAACCGGGGCGAGTGT |
| 500 | CL-57931 | Heavy chain | Nucleotide sequence of heavy | CAGGTCCAACTTGTGCAGTCTGGGGCTGAGGTGCAGAAGCCTGGGGCCTCAGTGAA |
| | | | chain | GGTTTCCTGCAAGGCTTCTGGATACACCTTCACTTCATATGCTTTGCATTGGGTCGC |
| | | | | CAGGCCCCCGGACAAAGGCTTGAGTGGCTGGGATGGATCAGCGCTGCCAATGGTAA |
| | | | | CACAGATTATTCATGGAAGTTCCAGGGCAGAGTCACCCTTACCAGGGACACATCCGC |
| | | | | AAACACAGTCTACATGGAACTGAACAGTCTGACATCTGAGGACTCGGCTGTGTATTA |
| | | | | CTGTGCGAGAAGGAAACTATGGTCTCCTTTTGATATCTGGGGCCAAGGGACATTGG |
| | | | | TCACCGTCTCTTCAGCCAGCACCAAGGGCCCTTCCGTGTTCCCCCTGGCCCCTTGCAG |
| | | | | CAGGAGCACCTCCGAATCCACAGCTGCCCTGGGCTGTCTGGTGAAGGACTACTTTCC |
| | | | | CGAGCCCGTGACCGTGAGCTGGAACAGCGGCGCTCTGACATCCGGCGTCCACACCT |
| | | | | TTCCTGCCGTCCTGCAGTCCTCCGGCCTCTACTCCCTGTCCTCCGTGGTGACCGTGCC |
| | | | | TAGCTCCTCCCTCGGCACCAAGACCTACACCTGTAACGTGGACCACAAACCCTCCAA |
| | | | | CACCAAGGTGGACAAACGGGTCGAGAGCAAGTACGGCCCTCCCTGCCCTCCTTGTCC |
| | | | | TGCCCCCGAGTTCGAAGGCGGACCCAGCGTGTTCCTGTTCCCTCCTAAGCCCAAGGA |
| | | | | CACCCTCATGATCAGCCGGACACCCGAGGTGACCTGCGTGGTGGTGGATGTGAGCC |
| | | | | AGGAGGACCCTGAGGTCCAGTTCAACTGGTATGTGGATGGCGTGGAGGTGCACAA |
| | | | | CGCCAAGACAAAGCCCCGGGAAGAGCAGTTCAACTCCACCTACAGGGTGGTCAGCG |
| | | | | TGCTGACCGTGCTGCATCAGGACTGGCTGAACGGCAAGGAGTACAAGTGCAAGGTC |
| | | | | AGCAATAAGGGACTGCCCAGCAGCATCGAGAAGACCATCTCCAAGGCTAAAGGCCA |
| | | | | GCCCCGGGAACCTCAGGTGTACACCCTGCCTCCCAGCCAGGAGGAGATGACCAAGA |
| | | | | ACCAGGTGAGCCTGACCTGCCTGGTGAAGGGATTCTACCCTTCCGACATCGCCGTGG |
| | | | | AGTGGGAGTCCAACGGCCAGCCCGAGAACAATTATAAGACCACCCCTCCCGTCCTCG |
| | | | | ACAGCGACGGATCCTTCTTTCTGTACTCCAGGCTGACCGTGGATAAGTCCAGGTGGC |
| | | | | AGGAAGGCAACGTGTTCAGCTGCTCCGTGATGCACGAGGCCCTGCACAATCACTAC |
| | | | | ACCCAGAAGTCCCTGAGCCTGTCCCTGGGAAAG |
| 501 | CL-57931 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGTTGGGCCAGTCAAAGTCTTACCAACAGTTTCTTAGCCTGGTACCGGC |
| | | | | AGAAACCTGGCCAGGCTCCCAGGCTCCTCATCTCTGGTGCATTCAGCAGGGCCACTG |
| | | | | ACATCCCAGACAGGATCAGTGGCAGTGGATCTGGGACAGACTTCACTCTCACCATCA |
| | | | | ACAGACTGGAGCCTGAAGATTTTGCAGTGTATTACTGTCAGTACTATGGTACCTCAC |
| | | | | CGTGGACGTTCGGCCAAGGGACCAAGGTGGAAATCAAACGTACGGTGGCCGCTCCC |
| | | | | TCCGTGTTCATCTTCCCACCTTCCGACGAGCAGCTGAAGTCCGGCACCGCTTCTGTCG |
| | | | | TGTGCCTGCTGAACAACTTCTACCCCCGCGAGGCCAAGGTGCAGTGGAAGGTGGAC |
| | | | | AACGCCCTGCAGTCCGGCAACTCCCAGGAATCCGTGACCGAGCAGGACTCCAAGGA |
| | | | | CAGCACCTACTCCCTGTCCTCCACCCTGACCCTGTCCAAGGCCGACTACGAGAAGCA |
| | | | | CAAGGTGTACGCCTGCGAAGTGACCCACCAGGGCCTGTCTAGCCCCGTGACCAAGT |
| | | | | CTTTCAACCGGGGCGAGTGT |
| 502 | CL-57945 | Heavy chain | Nucleotide sequence of heavy | CAGGTCCACCTTGTTCAGTCAGGGGCAGAGGTGAAGAACCCTGGGGCCTCAGTGAA |
| | | | chain | GGTCTCCTGCAAGGCTTCTGGATACACCTTCACTACCTTTGCTATTCATTGGTTGCGC |
| | | | | CAGGCCCCCGGACAGAGGCTTGAGTGGATGGGATGGATCAACCCTGGCAATGTTAA |
| | | | | GACAGATTATTCGCAGAAGTTCCAGGGCAGAGTCACCATTAGCAGGGACACATCCG |
| | | | | CGACCACTGCCTACATGGAGCTGAGCAGCCTGAGATCTGAAGACACGGCTGTTTATT |
| | | | | ACTGTGCGAGAAGACAATTATGGTTACCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 503 | CL-57945 | Light chain | Nucleotide sequence of light | GAAATTGTGTTGACGCAGTCTCCAGGCACCCTGTCTTTGTCTCCAGGGGAGGGAGCC |
| | | | chain | ACCCTCTCCTGCAGGGCCAGTCAGAGTATTAGCAACAACTTCTTAGCCTGGTACCAA |
| | | | | CAGAAACCTGGCCAGGCTCCCAGACTCCTCATCTTTGGTGCATCCAGCAGGGCCACT |
| | | | | GCCATCCCAGACAGGTTCGTTGGCAGTGGGTCTGGGACAGACTTCACTCTCACCATC |
| | | | | ACCGGACTGGAGCCTGAAGATTTTGCAGTGTATCACTGTCAACACTATGGTGGTTCA |
| | | | | CCTTGGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 504 | CL-57945 | Heavy chain | Nucleotide sequence of heavy | CAGGTCCACCTTGTTCAGTCAGGGGCAGAGGTGAAGAACCCTGGGGCCTCAGTGAA |
| | | | chain alternative coding | GGTCTCCTGCAAGGCTTCTGGATACACCTTCACTACCTTTGCTATTCATTGGTTGCGC |
| | | | | CAGGCCCCCGGACAGAGGCTTGAGTGGATGGGATGGATCAACCCTGGCAATGTTAA |
| | | | | GACAGATTATTCGCAGAAGTTCCAGGGCAGAGTCACCATTAGCAGGGACACATCCG |
| | | | | CGACCACTGCCTACATGGAGCTGAGCAGCCTGAGATCTGAAGACACGGCTGTTTATT |
| | | | | ACTGTGCGAGAAGACAATTATGGTTACCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 505 | CL-57945 | Light chain | Nucleotide sequence of light | GAAATTGTGTTGACGCAGTCTCCAGGCACCCTGTCTTTGTCTCCAGGGGAGGGAGCC |
| | | | chain alternative coding | ACCCTCTCCTGCAGGGCCAGTCAGAGTATTAGCAACAACTTCTTAGCCTGGTACCAA |
| | | | | CAGAAACCTGGCCAGGCTCCCAGACTCCTCATCTTTGGTGCATCCAGCAGGGCCACT |
| | | | | GCCATCCCAGACAGGTTCGTTGGCAGTGGGTCTGGGACAGACTTCACTCTCACCATC |
| | | | | ACCGGACTGGAGCCTGAAGATTTTGCAGTGTATCACTGTCAACACTATGGTGGTTCA |
| | | | | CCTTGGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 506 | CL-58102 | Heavy chain | Nucleotide sequence of heavy | CAGGTCCAGCTTGTGCAGTCTGGGGCTGAGGTGAAGAAGCCTGGGGCCTCAGTGA |
| | | | chain | AAGTTTCCTGTAAGGCTTCTGGGTACAGTTTCACTAACTATGCTTTACATTGGGTGCG |
| | | | | CCAGGCCCCCGGACAAAGACTTGAGTGGATGGGATGGATCCACGCTGGTAATGGTA |
| | | | | AGACAGAATATGCACAGAAGTTCCAGGACAGAGTCACCATTAGTAGGGACATATCC |
| | | | | GCGATCACAGTTTACATGGAACTGAGCAGCCTGAGATCTGAAGACACGGCTGTTTAT |
| | | | | TATTGTGCGAGAAGACAGTTATGGTTACCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 507 | CL-58102 | Light chain | Nucleotide sequence of light | GAAATTGTGTTGACGCAGTCTCCAGACACCCTCTCTTTGTCTCCAGGGGAAACAGCC |
| | | | chain | AGTTTCTCCTGCAGGGCCGGTCAAATTATTATCAACAGACAGTTAGCCTGGTACCAG |
| | | | | CGGAGACCTGGCCAGGCTCCCCGGCTCCTCATCTATGGCGCGTCCAATAGGGTCACT |
| | | | | GGCATCCCAGACAGGTTCAGTGGCAGTGGGTCTGGGACAGACTTCACTCTCACGAT |
| | | | | CAATAGACTGGAGCCTGAAGATTTTGCAGTGTATTACTGTCAGCACTATGGTGGCTC |
| | | | | ACCTTGGACGTTCGGCCAAGGGACCAAGGTGGAAATCAAACGTACGGTGGCCGCTC |
| | | | | CCTCCGTGTTCATCTTCCCACCTTCCGACGAGCAGCTGAAGTCCGGCACCGCTTCTGT |
| | | | | CGTGTGCCTGCTGAACAACTTCTACCCCCGCGAGGCCAAGGTGCAGTGGAAGGTGG |
| | | | | ACAACGCCCTGCAGTCCGGCAACTCCCAGGAATCCGTGACCGAGCAGGACTCCAAG |
| | | | | GACAGCACCTACTCCCTGTCCTCCACCCTGACCCTGTCCAAGGCCGACTACGAGAAG |
| | | | | CACAAGGTGTACGCCTGCGAAGTGACCCACCAGGGCCTGTCTAGCCCCGTGACCAA |
| | | | | GTCTTTCAACCGGGGCGAGTGT |
| 508 | CL-58252 | Heavy chain | Nucleotide sequence of heavy | CAGGTCCAGTTTGTGCAGTCTGGGGCTGAGGTGAAGAAGCCTGGGGCCTCAGTGAA |
| | | | chain | GGTTTCCTGTAAGGCTTCTGGATACACCTTCACTAGCCATGCTATGCATTGGGTGCG |
| | | | | CCAGGCCCCCGGACAAAGGCTTGAGTGGATGGGATGGATCAACGCTGCCAATGGTA |
| | | | | AAACAGATTATTCACAGAACTTCCAGGGCAGAGTCACCATTACCAGGGACACATACG |
| | | | | CGAACACAGTCTACATGGAACTGAGCAGCCTGAGATCTGAAGACACGGCTGTGTAT |
| | | | | TACTGTGCGAGACGCCCTTACGGTGGTCCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 509 | CL-58252 | Light chain | Nucleotide sequence of light | GACATCCAGATGACCCAGTCTCCTTCCACCCTGTCTGCATCTGTAGGAGACAGAGTC |
| | | | chain | ACCATCACTTGCCGGGCCAGTCAGAGTATTAGTATCTGGTTGGCCTGGTATCAGCAG |
| | | | | AAACCAGGGAAAGCCCCTAAGCTCCTGATCTATAAGGCGTCTAGTTTAGAAAGTGG |
| | | | | GGTCCCATCAAGGTTCAGCGGCAGTGGATCTGGGACAGAATTCACTCTCACCATCAG |
| | | | | CAGCCTGCAGCCTGATGATTTTGCAACTTATTACTGTCAACAGTATAATCTTTATCCG |
| | | | | TGGACGTTCGGCCAAGGGACCAAGGTGGAAATCAAACGTACGGTGGCCGCTCCCTC |
| | | | | CGTGTTCATCTTCCCACCTTCCGACGAGCAGCTGAAGTCCGGCACCGCTTCTGTCGTG |
| | | | | TGCCTGCTGAACAACTTCTACCCCCGCGAGGCCAAGGTGCAGTGGAAGGTGGACAA |
| | | | | CGCCCTGCAGTCCGGCAACTCCCAGGAATCCGTGACCGAGCAGGACTCCAAGGACA |
| | | | | GCACCTACTCCCTGTCCTCCACCCTGACCCTGTCCAAGGCCGACTACGAGAAGCACA |
| | | | | AGGTGTACGCCTGCGAAGTGACCCACCAGGGCCTGTCTAGCCCCGTGACCAAGTCTT |
| | | | | TCAACCGGGGCGAGTGT |
| 510 | CL-58252 | Heavy chain | Nucleotide sequence of heavy | CAGGTCCAGTTTGTGCAGTCTGGGGCTGAGGTGAAGAAGCCTGGGGCCTCAGTGAA |
| | | | chain alternative coding | GGTTTCCTGTAAGGCTTCTGGATACACCTTCACTAGCCATGCTATGCATTGGGTGCG |
| | | | | CCAGGCCCCCGGACAAAGGCTTGAGTGGATGGGATGGATCAACGCTGCCAATGGTA |
| | | | | AAACAGATTATTCACAGAACTTCCAGGGCAGAGTCACCATTACCAGGGACACATACG |
| | | | | CGAACACAGTCTACATGGAACTGAGCAGCCTGAGATCTGAAGACACGGCTGTGTAT |
| | | | | TACTGTGCGAGACGCCCTTACGGTGGTCCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 511 | CL-58252 | Light chain | Nucleotide sequence of light | GACATCCAGATGACCCAGTCTCCTTCCACCCTGTCTGCATCTGTAGGAGACAGAGTC |
| | | | chain alternative coding | ACCATCACTTGCCGGGCCAGTCAGAGTATTAGTATCTGGTTGGCCTGGTATCAGCAG |
| | | | | AAACCAGGGAAAGCCCCTAAGCTCCTGATCTATAAGGCGTCTAGTTTAGAAAGTGG |
| | | | | GGTCCCATCAAGGTTCAGCGGCAGTGGATCTGGGACAGAATTCACTCTCACCATCAG |
| | | | | CAGCCTGCAGCCTGATGATTTTGCAACTTATTACTGTCAACAGTATAATCTTTATCCG |
| | | | | TGGACGTTCGGCCAAGGGACCAAGGTGGAAATCAAACGTACGGTGGCCGCTCCCTC |
| | | | | CGTGTTCATCTTCCCACCTTCCGACGAGCAGCTGAAGTCCGGCACCGCTTCTGTCGTG |
| | | | | TGCCTGCTGAACAACTTCTACCCCCGCGAGGCCAAGGTGCAGTGGAAGGTGGACAA |
| | | | | CGCCCTGCAGTCCGGCAACTCCCAGGAATCCGTGACCGAGCAGGACTCCAAGGACA |
| | | | | GCACCTACTCCCTGTCCTCCACCCTGACCCTGTCCAAGGCCGACTACGAGAAGCACA |
| | | | | AGGTGTACGCCTGCGAAGTGACCCACCAGGGCCTGTCTAGCCCCGTGACCAAGTCTT |
| | | | | TCAACCGGGGCGAGTGT |
| 512 | CL-58838 | Heavy chain | Nucleotide sequence of heavy | CAGGTGCAACTGGTGGAGTCTGGGGGAGGCTTGGTCAAGCCTGGAGGGTCCCTGA |
| | | | chain | GACTCTCCTGTGCAGCCTCTGGATTCACCTTCAGTGACTACTACATGAGTTGGATCCG |
| | | | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCAGCCAGCACCAAGGGCCCTTCCGTGTTCCCCCTGGCCCCTTGCAG |
| | | | | CAGGAGCACCTCCGAATCCACAGCTGCCCTGGGCTGTCTGGTGAAGGACTACTTTCC |
| | | | | CGAGCCCGTGACCGTGAGCTGGAACAGCGGCGCTCTGACATCCGGCGTCCACACCT |
| | | | | TTCCTGCCGTCCTGCAGTCCTCCGGCCTCTACTCCCTGTCCTCCGTGGTGACCGTGCC |
| | | | | TAGCTCCTCCCTCGGCACCAAGACCTACACCTGTAACGTGGACCACAAACCCTCCAA |
| | | | | CACCAAGGTGGACAAACGGGTCGAGAGCAAGTACGGCCCTCCCTGCCCTCCTTGTCC |
| | | | | TGCCCCCGAGTTCGAAGGCGGACCCAGCGTGTTCCTGTTCCCTCCTAAGCCCAAGGA |
| | | | | CACCCTCATGATCAGCCGGACACCCGAGGTGACCTGCGTGGTGGTGGATGTGAGCC |
| | | | | AGGAGGACCCTGAGGTCCAGTTCAACTGGTATGTGGATGGCGTGGAGGTGCACAA |
| | | | | CGCCAAGACAAAGCCCCGGGAAGAGCAGTTCAACTCCACCTACAGGGTGGTCAGCG |
| | | | | TGCTGACCGTGCTGCATCAGGACTGGCTGAACGGCAAGGAGTACAAGTGCAAGGTC |
| | | | | AGCAATAAGGGACTGCCCAGCAGCATCGAGAAGACCATCTCCAAGGCTAAAGGCCA |
| | | | | GCCCCGGGAACCTCAGGTGTACACCCTGCCTCCCAGCCAGGAGGAGATGACCAAGA |
| | | | | ACCAGGTGAGCCTGACCTGCCTGGTGAAGGGATTCTACCCTTCCGACATCGCCCGTGG |
| | | | | AGTGGGAGTCCAACGGCCAGCCCGAGAACAATTATAAGACCACCCCTCCCGTCCTCG |
| | | | | ACAGCGACGGATCCTTCTTTCTGTACTCCAGGCTGACCGTGGATAAGTCCAGGTGGC |
| | | | | AGGAAGGCAACGTGTTCAGCTGCTCCGTGATGCACGAGGCCCTGCACAATCACTAC |
| | | | | ACCCAGAAGTCCCTGAGCCTGTCCCTGGGAAAG |
| 513 | CL-58838 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCTTGTTGGGCCAGTCAGAGGGTTGTTTACAGATACTTAGCCTGGTACCAG |
| | | | | CGGAAACCTGGCCAGGCTCCCAGACTTCTCATTTATGGTGCATTCAACAGGGCCACT |
| | | | | GGCATCCCAGACAGGTTCAGTGGCAGTGGGTCTGGGACAGACTTCAGTCTCACTAT |
| | | | | CAGTAGACTGGAGCCTGAGGATTTTGCAGTTTATTACTGTCACCAATATGGTAGTTC |
| | | | | ACCACCGACGTTCGGCCAAGGGACCAAGGTGGAAATCAAACGTACGGTGGCCGCTC |
| | | | | CCTCCGTGTTCATCTTCCCACCTTCCGACGAGCAGCTGAAGTCCGGCACCGCTTCTGT |
| | | | | CGTGTGCCTGCTGAACAACTTCTACCCCCGCGAGGCCAAGGTGCAGTGGAAGGTGG |
| | | | | ACAACGCCCTGCAGTCCGGCAACTCCCAGGAATCCGTGACCGAGCAGGACTCCAAG |
| | | | | GACAGCACCTACTCCCTGTCCTCCACCCTGACCCTGTCCAAGGCCGACTACGAGAAG |
| | | | | CACAAGGTGTACGCCTGCGAAGTGACCCACCAGGGCCTGTCTAGCCCCGTGACCAA |
| | | | | GTCTTTCAACCGGGGCGAGTGT |
| 514 | CL-58838 | Heavy chain | Nucleotide sequence of heavy | CAAGTTCAGTTGGTTGAGTCTGGCGGCGGACTGGTTAAGCCTGGCGGATCTCTGAG |
| | | | chain alternative coding | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCATG |
| | | | | GACTCCCTGTACCTGCAGATGAACTCCCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTGTCCTCTGCCAGCACCAAGGGCCCTTCCGTGTTCCCCCTGGCCCCTTGCAGCAG |
| | | | | GAGCACCTCCGAATCCACAGCTGCCCTGGGCTGTCTGGTGAAGGACTACTTTCCCGA |
| | | | | GCCCGTGACCGTGAGCTGGAACAGCGGCGCTCTGACATCCGGCGTCCACACCTTTCC |
| | | | | TGCCGTCCTGCAGTCCTCCGGCCTCTACTCCCTGTCCTCCGTGGTGACCGTGCCTAGC |
| | | | | TCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 515 | CL-58838 | Light chain | Nucleotide sequence of light | GAAATTGTGCTGACTCAGTCCCCTGGCACACTGTCTTTGAGCCCTGGCGAGAGAGCT |
| | | | chain alternative coding | ACCCTGTCTTGTTGGGCCTCTCAGAGAGTGGTGTACAGATACCTGGCTTGGTATCAG |
| | | | | CGGAAGCCCGGCCAGGCTCCTAGACTGTTGATCTACGGCGCCTTCAACAGAGCCAC |
| | | | | AGGCATCCCTGACAGATTCTCCGGCTCTGGCTCTGGCACCGACTTCTCCCTGACTATC |
| | | | | TCTCGGCTGGAACCCGAGGACTTCGCCGTGTACTACTGTCACCAGTACGGCAGCAGC |
| | | | | CCTCCTACCTTTGGCCAGGGCACTAAGGTGGAAATCAAACGTACGGTGGCCGCTCCC |
| | | | | TCCGTGTTCATCTTCCCACCTTCCGACGAGCAGCTGAAGTCCGGCACCGCTTCTGTCG |
| | | | | TGTGCCTGCTGAACAACTTCTACCCCCGCGAGGCCAAGGTGCAGTGGAAGGTGGAC |
| | | | | AACGCCCTGCAGTCCGGCAACTCCCAGGAATCCGTGACCGAGCAGGACTCCAAGGA |
| | | | | CAGCACCTACTCCCTGTCCTCCACCCTGACCCTGTCCAAGGCCGACTACGAGAAGCA |
| | | | | CAAGGTGTACGCCTGCGAAGTGACCCACCAGGGCCTGTCTAGCCCCGTGACCAAGT |
| | | | | CTTTCAACCGGGGCGAGTGT |
| 516 | CL-58838 | Heavy chain | Nucleotide sequence of heavy | CAGGTTCAGCTGGTTGAATCTGGCGGCGGACTGGTTAAGCCTGGCGGATCTCTGAG |
| | | | chain alternative coding | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCATG |
| | | | | GACTCCCTGTACCTGCAGATGAACTCCCTGAGAGCCGAGGACACCGCCGTGTATAC |
| | | | | TGCGCCCGTAGATCCTCTGGATGGTACGACTATTGGGGCCAGGGCACCCTGGTCAC |
| | | | | AGTGTCCTCTGCTTCTACCAAGGGACCCAGCGTGTTCCCTCTGGCTCCTTGCTCCAGA |
| | | | | TCCACCTCCGAGTCTACAGCTGCTCTGGGCTGCCTGGTCAAGGACTACTTTCCTGAG |
| | | | | CCTGTGACCGTGTCCTGGAACTCTGGCGCTCTGACATCTGGCGTGCACACCTTTCCA |
| | | | | GCTGTGCTGCAGTCCTCCGGCCTGTACTCTCTGTCCTCTGTCGTGACCGTGCCTTCCT |
| | | | | CTAGCCTGGGCACCAAGACCTACACCTGTAATGTGGACCACAAGCCTTCCAACACCA |
| | | | | AGGTGGACAAGCGCGTGGAATCTAAGTACGGCCCTCCTTGTCCTCCATGTCCTGCTC |
| | | | | CAGAGTTTGAAGGCGGCCCTTCCGTGTTTCTGTTCCCTCCAAAGCCTAAGGACACCC |
| | | | | TGATGATCTCTCGGACCCCTGAAGTGACCTGCGTGGTGGTGGATGTGTCCCAAGAG |
| | | | | GACCCTGAGGTGCAGTTCAATTGGTACGTGGACGGCGTGGAAGTGCACAACGCCAA |
| | | | | GACCAAGCCTAGAGAGGAACAGTTCAACTCCACCTACAGAGTGGTGTCCGTGCTGA |
| | | | | CCGTGCTGCACCAGGATTGGCTGAACGGCAAAGAGTACAAGTGCAAGGTGTCCAAC |
| | | | | AAGGGCCTGCCTAGCTCCATCGAAAAGACCATCAGCAAGGCCAAGGGCCAGCCTCG |
| | | | | AGAACCCCAGGTTTACACCCTGCCTCCAAGCCAAGAGGAAATGACCAAGAACCAGG |
| | | | | TGTCCCTGACCTGCCTCGTGAAGGGATTCTACCCCTCCGATATCGCCGTGGAATGGG |
| | | | | AGTCTAATGGCCAGCCAGAGAACAACTACAAGACAACCCCTCCTGTGCTGGACTCCG |
| | | | | ACGGCTCCTTCTTTCTGTATTCCCGCCTGACCGTGGACAAGTCCAGATGGCAAGAGG |
| | | | | GCAACGTGTTCTCCTGCAGCGTGATGCACGAGGCCCTGCACAATCACTACACCCAGA |
| | | | | AGTCCCTGTCTCTGTCCCTGGGCAAG |
| 517 | CL-58838 | Light chain | Nucleotide sequence of light | GAGATCGTGCTGACCCAGTCTCCTGGCACACTGTCACTGTCTCCAGGCGAGAGAGCT |
| | | | chain alternative coding | ACCCTGTCTTGTTGGGCCTCTCAGAGAGTGGTGTACAGATACCTGGCTTGGTATCAG |
| | | | | CGGAAGCCCGGCCAGGCTCCTAGACTGTTGATCTACGGCGCCTTCAACAGAGCCAC |
| | | | | AGGCATCCCTGACAGATTCTCCGGCTCTGGCTCTGGCACCGACTTCTCCCTGACTATC |
| | | | | TCTCGGCTGGAACCCGAGGACTTCGCCGTGTACTACTGTCACCAGTACGGCAGCAGC |
| | | | | CCTCCTACCTTTGGCCAGGGCACAAAGGTGGAAATCAAGCGTACGGTGGCCGCTCC |
| | | | | CAGCGTGTTCATCTTCCCCCCAAGCGACGAGCAGCTGAAGAGCGGCACCGCCAGCG |
| | | | | TGGTGTGTCTGCTGAACAACTTCTACCCCAGGGAGGCCAAGGTGCAGTGGAAGGTG |
| | | | | GACAACGCCCTGCAGAGCGGCAACAGCCAGGAGAGCGTCACCGAGCAGGACAGCA |
| | | | | AGGACTCCACCTACAGCCTGAGCAGCACCCTGACCCTGAGCAAGGCCGACTACGAG |
| | | | | AAGCACAAGGTGTACGCCTGTGAGGTGACCCACCAGGGCCTGTCCAGCCCCGTGAC |
| | | | | CAAGAGCTTCAACAGGGGCGAGTGC |
| 518 | CL-58838 | Heavy chain | Nucleotide sequence of heavy | CAGGTGCAACTGGTGGAGTCTGGGGGAGGCTTGGTCAAGCCTGGAGGGTCCCTGA |
| | | | chain IgG4-PE Version A | GACTCTCCTGTGCAGCCTCTGGATTCACCTTCAGTGACTACTACATGAGTTGGATCCG |
| | | | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCAGCCTCCACCAAGGGCCCATCCGTCTTCCCCCTGGCGCCCTGCTC |
| | | | | CAGGAGCACCTCCGAGAGCACGGCCGCCCTGGGCTGCCTGGTCAAGGACTACTTCC |
| | | | | CCGAACCAGTGACGGTGTCGTGGAACTCAGGCGCCCTGACCAGCGGCGTGCACACC |
| | | | | TTCCCGGCTGTCCTACAGTCCTCAGGACTCTACTCCCTCAGCAGCGTGGTGACCGTG |
| | | | | CCCTCCAGCAGCTTGGGCACGAAGACCTACACCTGCAACGTAGATCACAAGCCCAGC |
| | | | | AACACCAAGGTGGACAAGAGAGTTGAGTCCAAATATGGTCCCCCATGCCCACCATG |
| | | | | CCCAGCGCCTGAATTTGAGGGGGGACCATCAGTCTTCCTGTTCCCCCCAAAACCCAA |
| | | | | GGACACTCTCATGATCTCCCGGACCCCTGAGGTCACGTGCGTGGTGGTGGACGTGA |
| | | | | GCCAGGAAGACCCCGAGGTCCAGTTCAACTGGTACGTGGATGGCGTGGAGGTGCA |
| | | | | TAATGCCAAGACAAAGCCGCGGGAGGAGCAGTTCAACAGCACGTACCGTGTGGTCA |
| | | | | GCGTCCTCACCGTCCTGCACCAGGACTGGCTGAACGGCAAGGAGTACAAGTGCAAG |
| | | | | GTCTCCAACAAAGGCCTCCCGTCATCGATCGAGAAAACCATCTCCAAAGCCAAAGGG |
| | | | | CAGCCCCGAGAGCCACAGGTGTACACCCTGCCCCCATCCCAGGAGGAGATGACCAA |
| | | | | GAACCAGGTCAGCCTGACCTGCCTGGTCAAAGGCTTCTACCCCAGCGACATCGCCGT |
| | | | | GGAGTGGGAGAGCAATGGGCAGCCGGAGAACAACTACAAGACCACGCCTCCCGTG |
| | | | | CTGGACTCCGACGGATCCTTCTTCCTCTACAGCAGGCTAACCGTGGACAAGAGCAGG |
| | | | | TGGCAGGAGGGGAATGTCTTCTCATGCTCCGTGATGCATGAGGCTCTGCACAACCA |
| | | | | CTACACACAGAAGAGCCTCTCCCTGTCTCTGGGTAAA |
| 519 | CL-58838 | Heavy chain | Nucleotide sequence of heavy | CAGGTGCAACTGGTGGAGTCTGGGGGAGGCTTGGTCAAGCCTGGAGGGTCCCTGA |
| | | | chain IgG4-PE Version B | GACTCTCCTGTGCAGCCTCTGGATTCACCTTCAGTGACTACTACATGAGTTGGATCCG |
| | | | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCAGCCTCCACCAAGGGACCTAGCGTGTTCCCTCTCGCCCCCTGTTC |
| | | | | CAGGTCCACAAGCGAGTCCACCGCTGCCCTCGGCTGTCTGGTGAAAGACTACTTTCC |
| | | | | CGAGCCCGTGACCGTCTCCTGGAATAGCGGAGCCCTGACCTCCGGCGTGCACACATT |
| | | | | TCCCGCCGTGCTGCAGAGCAGCGGACTGTATAGCCTGAGCAGCGTGGTGACCGTGC |
| | | | | CCAGCTCCAGCCTCGGCACCAAAACCTACACCTGCAACGTGGACCACAAGCCCTCCA |
| | | | | ACACCAAGGTGGACAAGCGGGTGGAGAGCAAGTACGGCCCCCCTTGCCCTCCTTGT |
| | | | | CCTGCCCCTGAGTTCGAGGGAGGACCCTCCGTGTTCCTGTTTCCCCCCAAACCCAAG |
| | | | | GACACCCTGATGATCTCCCGGACACCCGAGGTGACCTGTGTGGTCGTGGACGTCAG |
| | | | | CCAGGAGGACCCCGAGGTGCAGTTCAACTGGTATGTGGACGGCGTGGAGGTGCAC |
| | | | | AATGCCAAAACCAAGCCCAGGGAGGAGCAGTTCAATTCCACCTACAGGGTGGTGAG |
| | | | | CGTGCTGACCGTCCTGCATCAGGATTGGCTGAACGGCAAGGAGTACAAGTGCAAGG |
| | | | | TGTCCAACAAGGGACTGCCCAGCTCCATCGAGAAGACCATCAGCAAGGCTAAGGGC |
| | | | | CAGCCGAGGGAGCCCCAGGTGTATACCCTGCCTCCTAGCCAGGAAGAGATGACCAA |
| | | | | GAACCAAGTGTCCCTGACCTGCCTGGTGAAGGGATTCTACCCCTCCGACATCGCCGT |
| | | | | GGAGTGGGAGAGCAATGGCCAGCCCGAGAACAACTACAAAACAACCCCTCCCGTGC |
| | | | | TCGATAGCGACGGCAGCTTCTTTCTCTACAGCCGGCTGACAGTGGACAAGAGCAGG |
| | | | | TGGCAGGAGGGCAACGTGTTCTCCTGTTCCGTGATGCACGAGGCCCTGCACAATCA |
| | | | | CTACACCCAGAAGAGCCTCTCCCTGTCCCTGGGCAAG |
| 520 | CL-58838 | Heavy chain | Nucleotide sequence of V_{H} | CAGGTTCAGCTGGTTGAATCTGGCGGCGGACTGGTTAAGCCTGGCGGATCTCTGAG |
| | | variable | alternative coding | ACTGTCTTGTGCCGCCTCTGGCTTCACCTTCTCCGACTACTACATGTCCTGGATCAGA |
| | | region | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCATG |
| | | | | GACTCCCTGTACCTGCAGATGAACTCCCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTGTCCTCT |
| 521 | CL-58838 | Heavy chain | Nucleotide sequence of V_{H} | CAAGTTCAGTTGGTTGAGTCTGGCGGCGGACTGGTTAAGCCTGGCGGATCTCTGAG |
| | | variable | alternative coding | ACTGTCTTGTGCCGCCTCTGGCTTCACCTTCTCCGACTACTACATGTCCTGGATCAGA |
| | | region | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCATG |
| | | | | GACTCCCTGTACCTGCAGATGAACTCCCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTGTCCTCT |
| 522 | CL-58838 | Light chain | Nucleotide sequence of V_{L} | GAGATCGTGCTGACCCAGTCTCCTGGCACACTGTCACTGTCTCCAGGCGAGAGAGCT |
| | | variable | alternative coding | ACCCTGTCTTGTTGGGCCTCTCAGAGAGTGGTGTACAGATACCTGGCTTGGTATCAG |
| | | region | | CGGAAGCCCGGCCAGGCTCCTAGACTGTTGATCTACGGCGCCTTCAACAGAGCCAC |
| | | | | AGGCATCCCTGACAGATTCTCCGGCTCTGGCTCTGGCACCGACTTCTCCCTGACTATC |
| | | | | TCTCGGCTGGAACCCGAGGACTTCGCCGTGTACTACTGTCACCAGTACGGCAGCAGC |
| | | | | CCTCCTACCTTTGGCCAGGGCACAAAGGTGGAAATCAAG |
| 523 | CL-58838 | Light chain | Nucleotide sequence of V_{L} | GAAATTGTGCTGACTCAGTCCCCTGGCACACTGTCTTTGAGCCCTGGCGAGAGAGCT |
| | | variable | alternative coding | ACCCTGTCTTGTTGGGCCTCTCAGAGAGTGGTGTACAGATACCTGGCTTGGTATCAG |
| | | region | | CGGAAGCCCGGCCAGGCTCCTAGACTGTTGATCTACGGCGCCTTCAACAGAGCCAC |
| | | | | AGGCATCCCTGACAGATTCTCCGGCTCTGGCTCTGGCACCGACTTCTCCCTGACTATC |
| | | | | TCTCGGCTGGAACCCGAGGACTTCGCCGTGTACTACTGTCACCAGTACGGCAGCAGC |
| | | | | CCTCCTACCTTTGGCCAGGGCACTAAGGTGGAAATCAAA |
| 524 | CL-58851 | Heavy chain | Nucleotide sequence of heavy | CAGGTCCAACTTGTGCAGTCTGGGGCTGAGGTGAAGAAGCCTGGGGCCTCAGTGAA |
| | | | chain | GGTTTCCTGCAAGGCTACTGGATTCACCTTCATTACCTATGCTTTCCATTGGGTGCGC |
| | | | | CAGGCCCCCGGACAAAGGTTTGAGTGGATGGGATGGATCAACGTTGGCAATGGTAA |
| | | | | CAGAGAATATTCACAGAAGTTCCAGGACAGAGTCACCATTACCAGGGACACATCCG |
| | | | | CGACCACAGTCTACATGGAACTGAACAGCCTGAAATCTGAAGACACGGCTATGTATT |
| | | | | TCTGTGCGAGACGCCCCCTCTGGGGTCCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 525 | CL-58851 | Light chain | Nucleotide sequence of light | GAAATTGTTTTGACACAGTCTCCAGGCACCCTGTCTTTGTCTCCAGGGGAAAGAGCC |
| | | | chain | ACCCTCTCCTGCAGGGCCAGTCAGATTTTTAGCAACACCTTCTTAGCCTGGTACCAGC |
| | | | | AGAAACCTGGCCAGGCTCCCAGGCTCCTCGTGTATGGTGCATCCAAGAGGGCCACT |
| | | | | GCCATCCCAGACAGGTTTAGTGGCAGTGGGTCTGGGACAGACTTCATTCTCACCATC |
| | | | | AACAGACTGGAGCCTGAAGATTTTGCAGTATATTACTGTCAACACTATGGTGGGTCA |
| | | | | CCGTGGACGTTCGGCCGAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 526 | CL-58851 | Heavy chain | Nucleotide sequence of heavy | CAGGTCCAACTTGTGCAGTCTGGGGCTGAGGTGAAGAAGCCTGGGGCCTCAGTGAA |
| | | | chain alternative coding | GGTTTCCTGCAAGGCTACTGGATTCACCTTCATTACCTATGCTTTCCATTGGGTGCGC |
| | | | | CAGGCCCCCGGACAAAGGTTTGAGTGGATGGGATGGATCAACGTTGGCAATGGTAA |
| | | | | CAGAGAATATTCACAGAAGTTCCAGGACAGAGTCACCATTACCAGGGACACATCCG |
| | | | | CGACCACAGTCTACATGGAACTGAACAGCCTGAAATCTGAAGACACGGCTATGTATT |
| | | | | TCTGTGCGAGACGCCCCCTCTGGGGTCCCTTTGACTACTGGGGCCAGGGAACCCTG |
| | | | | GTCACCGTCTCCTCAGCCAGCACCAAGGGCCCTTCCGTGTTCCCCCTGGCCCCTTGCA |
| | | | | GCAGGAGCACCTCCGAATCCACAGCTGCCCTGGGCTGTCTGGTGAAGGACTACTTTC |
| | | | | CCGAGCCCGTGACCGTGAGCTGGAACAGCGGCGCTCTGACATCCGGCGTCCACACC |
| | | | | TTTCCTGCCGTCCTGCAGTCCTCCGGCCTCTACTCCCTGTCCTCCGTGGTGACCGTGC |
| | | | | CTAGCTCCTCCCTCGGCACCAAGACCTACACCTGTAACGTGGACCACAAACCCTCCA |
| | | | | ACACCAAGGTGGACAAACGGGTCGAGAGCAAGTACGGCCCTCCCTGCCCTCCTTGT |
| | | | | CCTGCCCCCGAGTTCGAAGGCGGACCCAGCGTGTTCCTGTTCCCTCCTAAGCCCAAG |
| | | | | GACACCCTCATGATCAGCCGGACACCCGAGGTGACCTGCGTGGTGGTGGATGTGAG |
| | | | | CCAGGAGGACCCTGAGGTCCAGTTCAACTGGTATGTGGATGGCGTGGAGGTGCACA |
| | | | | ACGCCAAGACAAAGCCCCGGGAAGAGCAGTTCAACTCCACCTACAGGGTGGTCAGC |
| | | | | GTGCTGACCGTGCTGCATCAGGACTGGCTGAACGGCAAGGAGTACAAGTGCAAGGT |
| | | | | CAGCAATAAGGGACTGCCCAGCAGCATCGAGAAGACCATCTCCAAGGCTAAAGGCC |
| | | | | AGCCCCGGGAACCTCAGGTGTACACCCTGCCTCCCAGCCAGGAGGAGATGACCAAG |
| | | | | AACCAGGTGAGCCTGACCTGCCTGGTGAAGGGATTCTACCCTTCCGACATCGCCGTG |
| | | | | GAGTGGGAGTCCAACGGCCAGCCCGAGAACAATTATAAGACCACCCCTCCCGTCCTC |
| | | | | GACAGCGACGGATCCTTCTTTCTGTACTCCAGGCTGACCGTGGATAAGTCCAGGTGG |
| | | | | CAGGAAGGCAACGTGTTCAGCTGCTCCGTGATGCACGAGGCCCTGCACAATCACTA |
| | | | | CACCCAGAAGTCCCTGAGCCTGTCCCTGGGAAAG |
| 527 | CL-58851 | Light chain | Nucleotide sequence of light | GAAATTGTTTTGACACAGTCTCCAGGCACCCTGTCTTTGTCTCCAGGGGAAAGAGCC |
| | | | chain alternative coding | ACCCTCTCCTGCAGGGCCAGTCAGATTTTTAGCAACACCTTCTTAGCCTGGTACCAGC |
| | | | | AGAAACCTGGCCAGGCTCCCAGGCTCCTCGTGTATGGTGCATCCAAGAGGGCCACT |
| | | | | GCCATCCCAGACAGGTTTAGTGGCAGTGGGTCTGGGACAGACTTCATTCTCACCATC |
| | | | | AACAGACTGGAGCCTGAAGATTTTGCAGTATATTACTGTCAACACTATGGTGGGTCA |
| | | | | CCGTGGACGTTCGGCCGAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 528 | CL-75183 | Heavy chain | Nucleotide sequence of heavy | CAGGTCCAGCTTGTGCAGTCTGGGGCTGAGGTGAAGAAGCCTGGGGCCTCAGTGA |
| | | | chain | AGGTTTCCTGCAAGGCTTCTGGATACACCTTCACTAGCCATGCTATACATTGGGTGC |
| | | | | GCCAGGCCCCCGGACAACGGCTTGAGTGGATGGGATGGATCCACGCTGGCAATGG |
| | | | | TAACTCAAAACAGTCACAGAACTTCCAGGACAGAGTCACCATTACCAGGGACATC |
| | | | | CGCGAGCGCAGCCTACATGGAGCTGAGCAGCCTGAGATCTGAAGACACGGCTGTAT |
| | | | | ATTACTGTGCGAGACGGGCCATAATGGCCCCGTTTGACCTCTGGGGCCAGGGAACC |
| | | | | CTGGTCACCGTCTCCTCAGCCAGCACCAAGGGCCCTTCCGTGTTCCCCCTGGCCCCTT |
| | | | | GCAGCAGGAGCACCTCCGAATCCACAGCTGCCCTGGGCTGTCTGGTGAAGGACTAC |
| | | | | TTTCCCGAGCCCGTGACCGTGAGCTGGAACAGCGGCGCTCTGACATCCGGCGTCCA |
| | | | | CACCTTTCCTGCCGTCCTGCAGTCCTCCGGCCTCTACTCCCTGTCCTCCGTGGTGACC |
| | | | | GTGCCTAGCTCCTCCCTCGGCACCAAGACCTACACCTGTAACGTGGACCACAAACCC |
| | | | | TCCAACACCAAGGTGGACAAACGGGTCGAGAGCAAGTACGGCCCTCCCTGCCCTCC |
| | | | | TTGTCCTGCCCCCGAGTTCGAAGGCGGACCCAGCGTGTTCCTGTTCCCTCCTAAGCCC |
| | | | | AAGGACACCCTCATGATCAGCCGGACACCCGAGGTGACCTGCGTGGTGGTGGATGT |
| | | | | GAGCCAGGAGGACCCTGAGGTCCAGTTCAACTGGTATGTGGATGGCGTGGAGGTG |
| | | | | CACAACGCCAAGACAAAGCCCCGGGAAGAGCAGTTCAACTCCACCTACAGGGTGGT |
| | | | | CAGCGTGCTGACCGTGCTGCATCAGGACTGGCTGAACGGCAAGGAGTACAAGTGCA |
| | | | | AGGTCAGCAATAAGGGACTGCCCAGCAGCATCGAGAAGACCATCTCCAAGGCTAAA |
| | | | | GGCCAGCCCCGGGAACCTCAGGTGTACACCCTGCCTCCCAGCCAGGAGGAGATGAC |
| | | | | CAAGAACCAGGTGAGCCTGACCTGCCTGGTGAAGGGATTCTACCCTTCCGACATCGC |
| | | | | CGTGGAGTGGGAGTCCAACGGCCAGCCCGAGAACAATTATAAGACCACCCCTCCCG |
| | | | | TCCTCGACAGCGACGGATCCTTCTTTCTGTACTCCAGGCTGACCGTGGATAAGTCCA |
| | | | | GGTGGCAGGAAGGCAACGTGTTCAGCTGCTCCGTGATGCACGAGGCCCTGCACAAT |
| | | | | CACTACACCCAGAAGTCCCTGAGCCTGTCCCTGGGAAAG |
| 529 | CL-75183 | Light chain | Nucleotide sequence of light | GACATCCAGATGACCCAGTCTCCTTCCACCCTGTCTGCATCTGTAGGAGACAGAGTC |
| | | | chain | ACCATCACTTGCCGGGCCAGTCAGAGTATTAATAACTGGTTGGCCTGGTATCAGCAG |
| | | | | AAACCAGGGAAAGCCCCTAACCTCCTGATCTATAAGGCGTCTAGTTTAGAAAGTGG |
| | | | | GGTCCCATCAAGGTTCAGCGGCAGTGGATCTGGGACAGAATTCACTCTCACCATCAA |
| | | | | CAGCCTGCAGCCTGATGACTTTGCAACTTATTACTGCCAACAGTATTATAGTTCTTGG |
| | | | | ACGTTCGGCCAAGGGACCAAGGTGGAAATCAAACGTACGGTGGCCGCTCCCTCCGT |
| | | | | GTTCATCTTCCCACCTTCCGACGAGCAGCTGAAGTCCGGCACCGCTTCTGTCGTGTGC |
| | | | | CTGCTGAACAACTTCTACCCCCGCGAGGCCAAGGTGCAGTGGAAGGTGGACAACGC |
| | | | | CCTGCAGTCCGGCAACTCCCAGGAATCCGTGACCGAGCAGGACTCCAAGGACAGCA |
| | | | | CCTACTCCCTGTCCTCCACCCTGACCCTGTCCAAGGCCGACTACGAGAAGCACAAGG |
| | | | | TGTACGCCTGCGAAGTGACCCACCAGGGCCTGTCTAGCCCCGTGACCAAGTCTTTCA |
| | | | | ACCGGGGCGAGTGT |
| 530 | CL-75500 | Heavy chain | Nucleotide sequence of heavy | CAGGTCCAGCTTGTGCAGTCTGGGGCTGAGGTGAAGAAGCCTGGGGCCTCAGTGA |
| | | | chain | AGGTTTCCTGCAAGGCTTCTGGATACACCTTCACTAGCTATGCTGTCCATTGGGTGC |
| | | | | GCCAGGCCCCCGGACAAAGGCTTGAGTGGATGGGATGGATCAACGCTGGCAATGG |
| | | | | TAACACAAAATTTTCACAGAAGTTCCAGGGCAGAATCACCATTACCAGGGACACATC |
| | | | | CGCGAGCACAACCTACATGGAGTTGAATAGTCTGAGATCTGAAGACACGGCTGTGT |
| | | | | ATTATTGTGCGAGAAGGGGGTTCGGGGAGCCATTTGACTACTGGGGCCAGGGAAC |
| | | | | CCTGGTCACCGTCTCCTCAGCCAGCACCAAGGGCCCTTCCGTGTTCCCCCTGGCCCT |
| | | | | TGCAGCAGGAGCACCTCCGAATCCACAGCTGCCCTGGGCTGTCTGGTGAAGGACTA |
| | | | | CTTTCCCGAGCCCGTGACCGTGAGCTGGAACAGCGGCGCTCTGACATCCGGCGTCC |
| | | | | ACACCTTTCCTGCCGTCCTGCAGTCCTCCGGCCTCTACTCCCTGTCCTCCGTGGTGAC |
| | | | | CGTGCCTAGCTCCTCCCTCGGCACCAAGACCTACACCTGTAACGTGGACCACAAACC |
| | | | | CTCCAACACCAAGGTGGACAAACGGGTCGAGAGCAAGTACGGCCCTCCCTGCCCTC |
| | | | | CTTGTCCTGCCCCCGAGTTCGAAGGCGGACCCAGCGTGTTCCTGTTCCCTCCTAAGCC |
| | | | | CAAGGACACCCTCATGATCAGCCGGACACCCGAGGTGACCTGCGTGGTGGTGGATG |
| | | | | TGAGCCAGGAGGACCCTGAGGTCCAGTTCAACTGGTATGTGGATGGCGTGGAGGT |
| | | | | GCACAACGCCAAGACAAAGCCCCGGGAAGAGCAGTTCAACTCCACCTACAGGGTGG |
| | | | | TCAGCGTGCTGACCGTGCTGCATCAGGACTGGCTGAACGGCAAGGAGTACAAGTGC |
| | | | | AAGGTCAGCAATAAGGGACTGCCCAGCAGCATCGAGAAGACCATCTCCAAGGCTAA |
| | | | | AGGCCAGCCCCGGGAACCTCAGGTGTACACCCTGCCTCCCAGCCAGGAGGAGATGA |
| | | | | CCAAGAACCAGGTGAGCCTGACCTGCCTGGTGAAGGGATTCTACCCTTCCGACATCG |
| | | | | CCGTGGAGTGGGAGTCCAACGGCCAGCCCGAGAACAATTATAAGACCACCCCTCCC |
| | | | | GTCCTCGACAGCGACGGATCCTTCTTTCTGTACTCCAGGCTGACCGTGGATAAGTCC |
| | | | | AGGTGGCAGGAAGGCAACGTGTTCAGCTGCTCCGTGATGCACGAGGCCCTGCACAA |
| | | | | TCACTACACCCAGAAGTCCCTGAGCCTGTCCCTGGGAAAG |
| 531 | CL-75500 | Light chain | Nucleotide sequence of light | GAAATAGTGATGACGCAGTCTCCAGCCACCCTGTCTGTGTCTCCAGGGGAAAGAGC |
| | | | chain | CACCCTCTCCTGCAGGGCCAGTCAGAGTATTAGTAACAACTTAGCCTGGTACCAACA |
| | | | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGCCT |
| | | | | TTCACTTTCGGCCCTGGGACCAAAGTGGATATCAAACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 532 | CL-75506 | Heavy chain | Nucleotide sequence of heavy | CAGGTCCAGCTTGTGCAGTCTGGGGCTGAGGTGAAGAAGCCTGGGGCCTCAGTGA |
| | | | chain | AGGTTTCCTGCAAGGCTTCTGGATACACCTTCACTAGCTATGCTATCCATTGGGTGCG |
| | | | | CCAGGCCCCCGGACAAAGGCTTGAGTGGATGGGATGGATCAACCCTGGCAATGGTA |
| | | | | ACACAAAATTTTCACAGAAGTTCCAGGGCAGAATCACCATTACCAGGGACACATCCG |
| | | | | CGAGCACAACCTACATGGAGCTGAACAGCCTGAGATCTGAAGACACGGCTGTGTAT |
| | | | | TACTGTGCGAGAAGGGGGTTCGGGGAGCCATTTGACTACTGGGGCCAGGGAACCC |
| | | | | TGGTCACCGTCTCCTCAGCCAGCACCAAGGGCCCTTCCGTGTTCCCCCTGGCCCTTG |
| | | | | CAGCAGGAGCACCTCCGAATCCACAGCTGCCCTGGGCTGTCTGGTGAAGGACTACT |
| | | | | TTCCCGAGCCCGTGACCGTGAGCTGGAACAGCGGCGCTCTGACATCCGGCGTCCAC |
| | | | | ACCTTTCCTGCCGTCCTGCAGTCCTCCGGCCTCTACTCCCTGTCCTCCGTGGTGACCG |
| | | | | TGCCTAGCTCCTCCCTCGGCACCAAGACCTACACCTGTAACGTGGACCACAAACCCTC |
| | | | | CAACACCAAGGTGGACAAACGGGTCGAGAGCAAGTACGGCCCTCCCTGCCCTCCTT |
| | | | | GTCCTGCCCCCGAGTTCGAAGGCGGACCCAGCGTGTTCCTGTTCCCTCCTAAGCCCA |
| | | | | AGGACACCCTCATGATCAGCCGGACACCCGAGGTGACCTGCGTGGTGGTGGATGTG |
| | | | | AGCCAGGAGGACCCTGAGGTCCAGTTCAACTGGTATGTGGATGGCGTGGAGGTGC |
| | | | | ACAACGCCAAGACAAAGCCCCGGGAAGAGCAGTTCAACTCCACCTACAGGGTGGTC |
| | | | | AGCGTGCTGACCGTGCTGCATCAGGACTGGCTGAACGGCAAGGAGTACAAGTGCAA |
| | | | | GGTCAGCAATAAGGGACTGCCCAGCAGCATCGAGAAGACCATCTCCAAGGCTAAAG |
| | | | | GCCAGCCCCGGGAACCTCAGGTGTACACCCTGCCTCCCAGCCAGGAGGAGATGACC |
| | | | | AAGAACCAGGTGAGCCTGACCTGCCTGGTGAAGGGATTCTACCCTTCCGACATCGCC |
| | | | | GTGGAGTGGGAGTCCAACGGCCAGCCCGAGAACAATTATAAGACCACCCCTCCCGT |
| | | | | CCTCGACAGCGACGGATCCTTCTTTCTGTACTCCAGGCTGACCGTGGATAAGTCCAG |
| | | | | GTGGCAGGAAGGCAACGTGTTCAGCTGCTCCGTGATGCACGAGGCCCTGCACAATC |
| | | | | ACTACACCCAGAAGTCCCTGAGCCTGTCCCTGGGAAAG |
| 533 | CL-75506 | Light chain | Nucleotide sequence of light | GAAATAGTGATGACGCAGTCTCCAGCCACCCTGTCTGTGTCTCCAGGGGAAAGAGC |
| | | | chain | CACCCTCTCCTGCAGGGCCAGTCAGAGTATTAGCAGCAACTTAGCCTGGTACCAACA |
| | | | | GAACCCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 534 | CL-75520 | Heavy chain | Nucleotide sequence of heavy | CAGGTCCAACTTGTGCAGTCTGGGGCTGAGGTGAAGAAGCCTGGGGCCTCAGTGAA |
| | | | chain | GGTTTCCTGCAAGGCTTCTGGATACACCTTCACTAGCTATGCTATGCATTGGGTGCGT |
| | | | | CAGGCCCCCGGACACAGGCTTGAGTGGTTGGGATGGATCAACGCTGGCAATGGTTA |
| | | | | CACAAAATATTCACAGAAATTCCAGGACAGAGTCGCCATTACCAGGGACACATCCGC |
| | | | | GAGCACAGCCTTCATGGAGCTGAGCAGCCTGAGATCTGAAGACACGGCTGTGTATT |
| | | | | ACTGTGCGAGAGATCGTATTACTATTATTCGGCCCTTTGACTACTGGGGCCAGGGAA |
| | | | | CCCTGGTCACCGTCTCCTCAGCCAGCACCAAGGGCCCTTCCGTGTTCCCCCTGGCCCC |
| | | | | TTGCAGCAGGAGCACCTCCGAATCCACAGCTGCCCTGGGCTGTCTGGTGAAGGACT |
| | | | | ACTTTCCCGAGCCCGTGACCGTGAGCTGGAACAGCGGCGCTCTGACATCCGGCGTC |
| | | | | CACACCTTTCCTGCCGTCCTGCAGTCCTCCGGCCTCTACTCCCTGTCCTCCGTGGTGA |
| | | | | CCGTGCCTAGCTCCTCCCTCGGCACCAAGACCTACACCTGTAACGTGGACCACAAAC |
| | | | | CCTCCAACACCAAGGTGGACAAACGGGTCGAGAGCAAGTACGGCCCTCCCTGCCCT |
| | | | | CCTTGTCCTGCCCCCGAGTTCGAAGGCGGACCCAGCGTGTTCCTGTTCCCTCCTAAGC |
| | | | | CCAAGGACACCCTCATGATCAGCCGGACACCCGAGGTGACCTGCGTGGTGGTGGAT |
| | | | | GTGAGCCAGGAGGACCCTGAGGTCCAGTTCAACTGGTATGTGGATGGCGTGGAAGG |
| | | | | TGCACAACGCCAAGACAAAGCCCCGGGAAGAGCAGTTCAACTCCACCTACAGGGTG |
| | | | | GTCAGCGTGCTGACCGTGCTGCATCAGGACTGGCTGAACGGCAAGGAGTACAAGTG |
| | | | | CAAGGTCAGCAATAAGGGACTGCCCAGCAGCATCGAGAAGACCATCTCCAAGGCTA |
| | | | | AAGGCCAGCCCCGGGAACCTCAGGTGTACACCCTGCCTCCCAGCCAGGAGGAGATG |
| | | | | ACCAAGAACCAGGTGAGCCTGACCTGCCTGGTGAAGGGATTCTACCCTTCCGACATC |
| | | | | GCCGTGGAGTGGGAGTCCAACGGCCAGCCCGAGAACAATTATAAGACCACCCCTCC |
| | | | | CGTCCTCGACAGCGACGGATCCTTCTTTCTGTACTCCAGGCTGACCGTGGATAAGTC |
| | | | | CAGGTGGCAGGAAGGCAACGTGTTCAGCTGCTCCGTGATGCACGAGGCCCTGCACA |
| | | | | ATCACTACACCCAGAAGTCCCTGAGCCTGTCCCTGGGAAAG |
| 535 | CL-75520 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGCAGGGCCAGTCAGAGTATTAGCAGCAGCTACTTAGCCTGGTTCCAG |
| | | | | CAAAAACCTGGCCAGGCTCCCAGGCTCCTCATCTATGGTGCATCCAGCAGGGCCACT |
| | | | | GGCATCCCAGACAGGTTCAGTGGCAATGGGTCTGGGACAGACTTCACTCTCACCATC |
| | | | | AGCAGACTGGAGCCTGAAGATTTTGCAGTGTATTACTGTCAGCTGTATGGTAGCCA |
| | | | | TTCACTTTCGGCCCTGGGACCAAAATGGATATTAAACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 536 | CL-75539 | Heavy chain | Nucleotide sequence of heavy | CAGGTCCAGCTTGTGCAGTCTGGGGCTGAGGTGAAGAAGCCTGGGGCCTCAGTGA |
| | | | chain | AAGTTTCCTGCAAGGCTTCTGGATACACCTTCACTAGCTATGCTATCCATTGGGTGCG |
| | | | | CCAGGCCCCCGGACAAAGGCTTGAGTGGATGGGATGGATCAACGTTGGCAATGGTA |
| | | | | AAACAAAATTTTCACAGAAGTTACAGGGCAGAATCACCATTACCAGGGACACATCCG |
| | | | | CGAGCACAACCTACATGGAGCTGAACAGCCTGAGATCTGAAGACACGGCTGTGTTT |
| | | | | TACTGTGCGAGAAGGGGGTTCGGGGAGCCATTTGACTACTGGGGCCAGGGAACCC |
| | | | | TGGTCACCGTCTCCTCAGCCAGCACCAAGGGCCCTTCCGTGTTCCCCCTGGCCCTTG |
| | | | | CAGCAGGAGCACCTCCGAATCCACAGCTGCCCTGGGCTGTCTGGTGAAGGACTACT |
| | | | | TTCCCGAGCCCGTGACCGTGAGCTGGAACAGCGGCGCTCTGACATCCGGCGTCCAC |
| | | | | ACCTTTCCTGCCGTCCTGCAGTCCTCCGGCCTCTACTCCCTGTCCTCCGTGGTGACCG |
| | | | | TGCCTAGCTCCTCCCTCGGCACCAAGACCTACACCTGTAACGTGGACCACAAACCCTC |
| | | | | CAACACCAAGGTGGACAAACGGGTCGAGAGCAAGTACGGCCCTCCCTGCCCTCCTT |
| | | | | GTCCTGCCCCCGAGTTCGAAGGCGGACCCAGCGTGTTCCTGTTCCCTCCTAAGCCCA |
| | | | | AGGACACCCTCATGATCAGCCGGACACCCGAGGTGACCTGCGTGGTGGTGGATGTG |
| | | | | AGCCAGGAGGACCCTGAGGTCCAGTTCAACTGGTATGTGGATGGCGTGGAGGTGC |
| | | | | ACAACGCCAAGACAAAGCCCCGGGAAGAGCAGTTCAACTCCACCTACAGGGTGGTC |
| | | | | AGCGTGCTGACCGTGCTGCATCAGGACTGGCTGAACGGCAAGGAGTACAAGTGCAA |
| | | | | GGTCAGCAATAAGGGACTGCCCAGCAGCATCGAGAAGACCATCTCCAAGGCTAAAG |
| | | | | GCCAGCCCCGGGAACCTCAGGTGTACACCCTGCCTCCCAGCCAGGAGGAGATGACC |
| | | | | AAGAACCAGGTGAGCCTGACCTGCCTGGTGAAGGGATTCTACCCTTCCGACATCGCC |
| | | | | GTGGAGTGGGAGTCCAACGGCCAGCCCGAGAACAATTATAAGACCACCCCTCCCGT |
| | | | | CCTCGACAGCGACGGATCCTTCTTTCTGTACTCCAGGCTGACCGTGGATAAGTCCAG |
| | | | | GTGGCAGGAAGGCAACGTGTTCAGCTGCTCCGTGATGCACGAGGCCCTGCACAATC |
| | | | | ACTACACCCAGAAGTCCCTGAGCCTGTCCCTGGGAAAG |
| 537 | CL-75539 | Light chain | Nucleotide sequence of light | GAAATAGTGATGACGCAGTCTCCAGCCACCCTGTCTGTGTCTCCAGGGGAAAGAGC |
| | | | chain | CACCCTCTCCTGCAGGGCCAGTCAGAGTATTAGTAGCAACTTAGCCTGGTACCAACA |
| | | | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 538 | CL-75565 | Heavy chain | Nucleotide sequence of heavy | CAGGTCCAGCTTGTGCAGTCTGGGGCTGAGGTGAAGAAGCCTGGGGCCTCAGTGA |
| | | | chain | AGGTTTCCTGCAAGGCTTCTGGATACACCTTCACTAGCTATGCTATCCATTGGGTGCG |
| | | | | CCAGGCCCCCGGACAAAGGCTTGAGTGGATGGGATGGATCAACGCTGGCAATGGT |
| | | | | AACACAAAATATTCACAGAAGTTCCAGGGCAGAATCACCATTACCAGGGACACATCC |
| | | | | GCGAGCACAGCCTACATGGAGCTGAGCAGCCTGAGATCTGAAGACACGGCTGTGTA |
| | | | | TTACTGTGCGAGAAGGGGGTTCGGGGAGCCATTTGACTACTGGGGCCAGGGAACC |
| | | | | CTGGTCACCGTCTCCTCAGCCAGCACCAAGGGCCCTTCCGTGTTCCCCCTGGCCCCTT |
| | | | | GCAGCAGGAGCACCTCCGAATCCACAGCTGCCCTGGGCTGTCTGGTGAAGGACTAC |
| | | | | TTTCCCGAGCCCGTGACCGTGAGCTGGAACAGCGGCGCTCTGACATCCGGCGTCCA |
| | | | | CACCTTTCCTGCCGTCCTGCAGTCCTCCGGCCTCTACTCCCTGTCCTCCGTGGTGACC |
| | | | | GTGCCTAGCTCCTCCCTCGGCACCAAGACCTACACCTGTAACGTGGACCACAAACCC |
| | | | | TCCAACACCAAGGTGGACAAACGGGTCGAGAGCAAGTACGGCCCTCCCTGCCCTCC |
| | | | | TTGTCCTGCCCCCGAGTTCGAAGGCGGACCCAGCGTGTTCCTGTTCCCTCCTAAGCCC |
| | | | | AAGGACACCCTCATGATCAGCCGGACACCCGAGGTGACCTGCGTGGTGGTGGATGT |
| | | | | GAGCCAGGAGGACCCTGAGGTCCAGTTCAACTGGTATGTGGATGGCGTGGAGGTG |
| | | | | CACAACGCCAAGACAAAGCCCCGGGAAGAGCAGTTCAACTCCACCTACAGGGTGGT |
| | | | | CAGCGTGCTGACCGTGCTGCATCAGGACTGGCTGAACGGCAAGGAGTACAAGTGCA |
| | | | | AGGTCAGCAATAAGGGACTGCCCAGCAGCATCGAGAAGACCATCTCCAAGGCTAAA |
| | | | | GGCCAGCCCCGGGAACCTCAGGTGTACACCCTGCCTCCCAGCCAGGAGGAGATGAC |
| | | | | CAAGAACCAGGTGAGCCTGACCTGCCTGGTGAAGGGATTCTACCCTTCCGACATCGC |
| | | | | CGTGGAGTGGGAGTCCAACGGCCAGCCCGAGAACAATTATAAGACCACCCCTCCCG |
| | | | | TCCTCGACAGCGACGGATCCTTCTTTCTGTACTCCAGGCTGACCGTGGATAAGTCCA |
| | | | | GGTGGCAGGAAGGCAACGTGTTCAGCTGCTCCGTGATGCACGAGGCCCTGCACAAT |
| | | | | CACTACACCCAGAAGTCCCTGAGCCTGTCCCTGGGAAAG |
| 539 | CL-75565 | Light chain | Nucleotide sequence of light | GAAATAGTGATGACGCAGTCTCCAGCCACCCTGTCTGTGTCTCCAGGGGAAAGAGC |
| | | | chain | CACCCTCTCCTGCAGGGCCAGTCAGAGTATTAGCAGCAACTTAGCCTGGTACCAACA |
| | | | | GAAACCTGGCCAGGCTCCCAGGCTCCTCATCTATGGTACATCCACCAGGGCCACTGG |
| | | | | TATCCCAGCCAGGTTCAGTGGCAGTGGGTCTGGGACAGAGTTCACTCTCACCATCAG |
| | | | | CACCCTGCAGTCTGAAGATTTTGCAGTTTATTACTGTCAGCAGTATAATATCTGGCCT |
| | | | | TTCACTTTCGGCCCTGGGACCAAAGTGGATATCAAACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 540 | CL-75714 | Heavy chain | Nucleotide sequence of heavy | CAGGTCCACCTTGTGCAGTCTGGGGCTGAGGTGAAGAAGCCTGGGGCCTCAGTGAA |
| | | | chain | GGTGTCCTGCAAGACTTCTGGATACACCTTCACCACCTATGCTATTCATTGGGTGCGC |
| | | | | CAGGCCCCCGGACAAGGGCTTGAGTGGATGGGATGGATCAACGCTGGCAATGGTA |
| | | | | GAACAGAATATTCAGAGAAGTTTCAGGGCAGAGTCACCATTACCAGGGACACTTCC |
| | | | | GCGAGTACAGTCTACATGGACCTGAGCAGCCTGAGATCTGGAGACACGGCTGTGTA |
| | | | | TTACTGTGCGAGAAGGGGATTCGGGGAGCCATTTGACCAATGGGGCCAGGGAACC |
| | | | | CTGGTCACCGTCTCCTCAGCCAGCACCAAGGGCCCTTCCGTGTTCCCCCTGGCCCCTT |
| | | | | GCAGCAGGAGCACCTCCGAATCCACAGCTGCCCTGGGCTGTCTGGTGAAGGACTAC |
| | | | | TTTCCCGAGCCCGTGACCGTGAGCTGGAACAGCGGCGCTCTGACATCCGGCGTCCA |
| | | | | CACCTTTCCTGCCGTCCTGCAGTCCTCCGGCCTCTACTCCCTGTCCTCCGTGGTGACC |
| | | | | GTGCCTAGCTCCTCCCTCGGCACCAAGACCTACACCTGTAACGTGGACCACAAACCC |
| | | | | TCCAACACCAAGGTGGACAAACGGGTCGAGAGCAAGTACGGCCCTCCCTGCCCTCC |
| | | | | TTGTCCTGCCCCCGAGTTCGAAGGCGGACCCAGCGTGTTCCTGTTCCCTCCTAAGCCC |
| | | | | AAGGACACCCTCATGATCAGCCGGACACCCGAGGTGACCTGCGTGGTGGTGGATGT |
| | | | | GAGCCAGGAGGACCCTGAGGTCCAGTTCAACTGGTATGTGGATGGCGTGGAGGTG |
| | | | | CACAACGCCAAGACAAAGCCCCGGGAAGAGCAGTTCAACTCCACCTACAGGGTGGT |
| | | | | CAGCGTGCTGACCGTGCTGCATCAGGACTGGCTGAACGGCAAGGAGTACAAGTGCA |
| | | | | AGGTCAGCAATAAGGGACTGCCCAGCAGCATCGAGAAGACCATCTCCAAGGCTAAA |
| | | | | GGCCAGCCCCGGGAACCTCAGGTGTACACCCTGCCTCCCAGCCAGGAGGAGATGAC |
| | | | | CAAGAACCAGGTGAGCCTGACCTGCCTGGTGAAGGGATTCTACCCTTCCGACATCGC |
| | | | | CGTGGAGTGGGAGTCCAACGGCCAGCCCGAGAACAATTATAAGACCACCCCTCCCG |
| | | | | TCCTCGACAGCGACGGATCCTTCTTTCTGTACTCCAGGCTGACCGTGGATAAGTCCA |
| | | | | GGTGGCAGGAAGGCAACGTGTTCAGCTGCTCCGTGATGCACGAGGCCCTGCACAAT |
| | | | | CACTACACCCAGAAGTCCCTGAGCCTGTCCCTGGGAAAG |
| 541 | CL-75714 | Light chain | Nucleotide sequence of light | GAAATAGTGATGACGCAGTCTCCAGCCACCCTGTCTGTGTCTCCAGGGGAAAGAGC |
| | | | chain | CACCCTCTCCTGCAGGGCCAGTCAGAGTGTTAGCAGCAACTTAGCCTGGTACCAGCA |
| | | | | CCAACCTGGCCAGGCTCCCAGGCTCCTCATCTATGGTGCATCCACCAGGGCCACTGG |
| | | | | TTTCCCACCCAGGTTCAGTGGCAGTGGGTCTGGGACAGACTTCACTCTCACCATCAA |
| | | | | CAGCCTGCAGTCTGAAGATTTTGCAGTTTATTACTGTCAGCAGTATAATAACTGGCCT |
| | | | | TTCATTTTCGGCCCTGGGACCAAACTGGATATCACACGTACGGTGGCCGCTCCCTCC |
| | | | | GTGTTCATCTTCCCACCTTCCGACGAGCAGCTGAAGTCCGGCACCGCTTCTGTCGTGT |
| | | | | GCCTGCTGAACAACTTCTACCCCCGCGAGGCCAAGGTGCAGTGGAAGGTGGACAAC |
| | | | | GCCCTGCAGTCCGGCAACTCCCAGGAATCCGTGACCGAGCAGGACTCCAAGGACAG |
| | | | | CACCTACTCCCTGTCCTCCACCCTGACCCTGTCCAAGGCCGACTACGAGAAGCACAA |
| | | | | GGTGTACGCCTGCGAAGTGACCCACCAGGGCCTGTCTAGCCCCGTGACCAAGTCTTT |
| | | | | CAACCGGGGCGAGTGT |
| 542 | CL-58722 | Heavy chain | Nucleotide sequence of heavy | CAGGTGCAACTGGTGGAGTCTGGGGGAGGCTTGGTCAAGCCTGGAGGGTCCCTGA |
| | | | chain | GACTCTCCTGTGCAGCCTCTGGATTCACCTTCAGTGACTACTACATGAGCTGGATCCG |
| | | | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | AGAACTCACTGTTTCTGCAAATGAACAGCCTGAGAGCCGAGGACACGGCCGTGTAT |
| | | | | TACTGTGCGAGAAGAAGCAGTGGCTGGTACGACTACTGGGGCCAGGGAACCCTGG |
| | | | | TCACCGTCTCCTCAGCCAGCACCAAGGGCCCTTCCGTGTTCCCCCTGGCCCCTTGCAG |
| | | | | CAGGAGCACCTCCGAATCCACAGCTGCCCTGGGCTGTCTGGTGAAGGACTACTTTCC |
| | | | | CGAGCCCGTGACCGTGAGCTGGAACAGCGGCGCTCTGACATCCGGCGTCCACACCT |
| | | | | TTCCTGCCGTCCTGCAGTCCTCCGGCCTCTACTCCCTGTCCTCCGTGGTGACCGTGCC |
| | | | | TAGCTCCTCCCTCGGCACCAAGACCTACACCTGTAACGTGGACCACAAACCCTCCAA |
| | | | | CACCAAGGTGGACAAACGGGTCGAGAGCAAGTACGGCCCTCCCTGCCCTCCTTGTCC |
| | | | | TGCCCCCGAGTTCGAAGGCGGACCCAGCGTGTTCCTGTTCCCTCCTAAGCCCAAGGA |
| | | | | CACCCTCATGATCAGCCGGACACCCGAGGTGACCTGCGTGGTGGTGGATGTGAGCC |
| | | | | AGGAGGACCCTGAGGTCCAGTTCAACTGGTATGTGGATGGCGTGGAGGTGCACAA |
| | | | | CGCCAAGACAAAGCCCCGGGAAGAGCAGTTCAACTCCACCTACAGGGTGGTCAGCG |
| | | | | TGCTGACCGTGCTGCATCAGGACTGGCTGAACGGCAAGGAGTACAAGTGCAAGGTC |
| | | | | AGCAATAAGGGACTGCCCAGCAGCATCGAGAAGACCATCTCCAAGGCTAAAGGCCA |
| | | | | GCCCCGGGAACCTCAGGTGTACACCCTGCCTCCCAGCCAGGAGGAGATGACCAAGA |
| | | | | ACCAGGTGAGCCTGACCTGCCTGGTGAAGGGATTCTACCCTTCCGACATCGCCGTGG |
| | | | | AGTGGGAGTCCAACGGCCAGCCCGAGAACAATTATAAGACCACCCCTCCCGTCCTCG |
| | | | | ACAGCGACGGATCCTTCTTTCTGTACTCCAGGCTGACCGTGGATAAGTCCAGGTGGC |
| | | | | AGGAAGGCAACGTGTTCAGCTGCTCCGTGATGCACGAGGCCCTGCACAATCACTAC |
| | | | | ACCCAGAAGTCCCTGAGCCTGTCCCTGGGAAAG |
| 543 | CL-58722 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGCAGGGCCAGTCAGAGTGTTAGCAGCAACTACTTAGCCTGGTACCAG |
| | | | | CAGAAACCTGGCCAGGCTCCCAGGCTCCTCATCTATGGTGCATCCATCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTCCTGTCACCAGTATGGTAGCTCA |
| | | | | CCTCCGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 544 | CL-58722 | Heavy chain | Nucleotide sequence of heavy | CAAGTTCAGTTGGTTGAGTCTGGCGGCGGACTGGTTAAGCCTGGCGGATCTCTGAG |
| | | | chain alternative coding | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCAAG |
| | | | | AACTCCCTGTTCCTGCAGATGAACAGCCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTTTCTAGTGCCAGCACCAAGGGCCCTTCCGTGTTCCCCCTGGCCCCTTGCAGCAG |
| | | | | GAGCACCTCCGAATCCACAGCTGCCCTGGGCTGTCTGGTGAAGGACTACTTTCCCGA |
| | | | | GCCCGTGACCGTGAGCTGGAACAGCGGCGCTCTGACATCCGGCGTCCACACCTTTCC |
| | | | | TGCCGTCCTGCAGTCCTCCGGCCTCTACTCCCTGTCCTCCGTGGTGACCGTGCCTAGC |
| | | | | TCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 545 | CL-58722 | Light chain | Nucleotide sequence of light | GAAATTGTGCTGACTCAGTCCCCTGGCACACTGTCTTTGAGCCCTGGCGAGAGAGCT |
| | | | chain alternative coding | ACCCTGTCCTGTAGAGCCTCTCAGTCCGTGTCCTCCAACTACCTGGCCTGGTATCAGC |
| | | | | AGAAGCCTGGACAGGCTCCCCGGCTGTTGATCTACGGCGCTTCTATCAGAGCCACA |
| | | | | GGCATCCCTGACCGGTTCTCCGGATCTGGCTCTGGCACCGATTTCACCCTGACCATCT |
| | | | | CTCGGCTGGAACCCGAGGATTTCGCCGTGTACTCTTGCCACCAGTACGGCTCTAGCC |
| | | | | CTCCTACCTTTGGACAGGGCACCAAGGTGGAAATCAAACGTACGGTGGCCGCTCCCT |
| | | | | CCGTGTTCATCTTCCCACCTTCCGACGAGCAGCTGAAGTCCGGCACCGCTTCTGTCGT |
| | | | | GTGCCTGCTGAACAACTTCTACCCCCGCGAGGCCAAGGTGCAGTGGAAGGTGGACA |
| | | | | ACGCCCTGCAGTCCGGCAACTCCCAGGAATCCGTGACCGAGCAGGACTCCAAGGAC |
| | | | | AGCACCTACTCCCTGTCCTCCACCCTGACCCTGTCCAAGGCCGACTACGAGAAGCAC |
| | | | | AAGGTGTACGCCTGCGAAGTGACCCACCAGGGCCTGTCTAGCCCCGTGACCAAGTC |
| | | | | TTTCAACCGGGGCGAGTGT |
| 546 | CL-58835 | Heavy chain | Nucleotide sequence of heavy | CAGGTGCAACTGGTGGAGTCTGGGGGAGGCTTGGTCAAGCCTGGAGGGTCCCTGA |
| | | | chain | GACTCTCCTGTGCAGCCTCTGGATTCACCTTCAGTGACTACTACATGAGTTGGATCCG |
| | | | | CCAGGCTCCAGGGAAGGGGCTGGAGTGGGTTTCATACATTAGTATTAGTGGTACTA |
| | | | | CCATATACTACGCAGACTCTGTGAAGGGCCGATTCACCATCTCCAGGGACAACGCCA |
| | | | | TGGACTCACTGTATCTGCAAATGAACAGCCTGAGAGCCGAGGACACGGCCGTGTAT |
| | | | | TACTGTGCGAGACGGAGCAGTGGCTGGTACGACTACTGGGGCCAGGGAACCCTGG |
| | | | | TCACCGTCTCCTCAGCCAGCACCAAGGGCCCTTCCGTGTTCCCCCTGGCCCCTTGCAG |
| | | | | CAGGAGCACCTCCGAATCCACAGCTGCCCTGGGCTGTCTGGTGAAGGACTACTTTCC |
| | | | | CGAGCCCGTGACCGTGAGCTGGAACAGCGGCGCTCTGACATCCGGCGTCCACACCT |
| | | | | TTCCTGCCGTCCTGCAGTCCTCCGGCCTCTACTCCCTGTCCTCCGTGGTGACCGTGCC |
| | | | | TAGCTCCTCCCTCGGCACCAAGACCTACACCTGTAACGTGGACCACAAACCCTCCAA |
| | | | | CACCAAGGTGGACAAACGGGTCGAGAGCAAGTACGGCCCTCCCTGCCCTCCTTGTCC |
| | | | | TGCCCCCGAGTTCGAAGGCGGACCCAGCGTGTTCCTGTTCCCTCCTAAGCCCAAGGA |
| | | | | CACCCTCATGATCAGCCGGACACCCGAGGTGACCTGCGTGGTGGTGGATGTGAGCC |
| | | | | AGGAGGACCCTGAGGTCCAGTTCAACTGGTATGTGGATGGCGTGGAGGTGCACAA |
| | | | | CGCCAAGACAAAGCCCCGGGAAGAGCAGTTCAACTCCACCTACAGGGTGGTCAGCG |
| | | | | TGCTGACCGTGCTGCATCAGGACTGGCTGAACGGCAAGGAGTACAAGTGCAAGGTC |
| | | | | AGCAATAAGGGACTGCCCAGCAGCATCGAGAAGACCATCTCCAAGGCTAAAGGCCA |
| | | | | GCCCCGGGAACCTCAGGTGTACACCCTGCCTCCCAGCCAGGAGGAGATGACCAAGA |
| | | | | ACCAGGTGAGCCTGACCTGCCTGGTGAAGGGATTCTACCCTTCCGACATCGCCGTGG |
| | | | | AGTGGGAGTCCAACGGCCAGCCCGAGAACAATTATAAGACCACCCCTCCCGTCCTCG |
| | | | | ACAGCGACGGATCCTTCTTTCTGTACTCCAGGCTGACCGTGGATAAGTCCAGGTGGC |
| | | | | AGGAAGGCAACGTGTTCAGCTGCTCCGTGATGCACGAGGCCCTGCACAATCACTAC |
| | | | | ACCCAGAAGTCCCTGAGCCTGTCCCTGGGAAAG |
| 547 | CL-58835 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCTTGTTGGGCCAGTCAGAGGGTTGTTTACAGATACTTAGCCTGGTACCAG |
| | | | | CGGAAACCTGGCCAGGCTCCCAGACTTCTCATTTATGGTGCATTCAACAGGGCCACT |
| | | | | GGCATCCCAGACAGGTTCAGTGGCAGTGGGTCTGGGACAGACTTCAGTCTCACTAT |
| | | | | CAGTAGACTGGAGCCTGAGGATTTTGCAGTTTATTACTGTCACCAATATGGTAGTTC |
| | | | | ACCACCGACGTTCGGCCAAGGGACCAAGGTGGAAATCAAACGTACGGTGGCCGCTC |
| | | | | CCTCCGTGTTCATCTTCCCACCTTCCGACGAGCAGCTGAAGTCCGGCACCGCTTCTGT |
| | | | | CGTGTGCCTGCTGAACAACTTCTACCCCCGCGAGGCCAAGGTGCAGTGGAAGGTGG |
| | | | | ACAACGCCCTGCAGTCCGGCAACTCCCAGGAATCCGTGACCGAGCAGGACTCCAAG |
| | | | | GACAGCACCTACTCCCTGTCCTCCACCCTGACCCTGTCCAAGGCCGACTACGAGAAG |
| | | | | CACAAGGTGTACGCCTGCGAAGTGACCCACCAGGGCCTGTCTAGCCCCGTGACCAA |
| | | | | GTCTTTCAACCGGGGCGAGTGT |
| 548 | CL-58835 | Heavy chain | Nucleotide sequence of heavy | CAAGTTCAGTTGGTTGAGTCTGGCGGCGGACTGGTTAAGCCTGGCGGATCTCTGAG |
| | | | chain alternative coding | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCACCACC |
| | | | | ATCTACTACGCCGACTCCGTGAAGGGCAGATTCACCATCTCCAGAGACAACGCCATG |
| | | | | GACTCCCTGTACCTGCAGATGAACTCCCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTTTCTAGTGCCAGCACCAAGGGCCCTTCCGTGTTCCCCCTGGCCCCTTGCAGCAG |
| | | | | GAGCACCTCCGAATCCACAGCTGCCCTGGGCTGTCTGGTGAAGGACTACTTTCCCGA |
| | | | | GCCCGTGACCGTGAGCTGGAACAGCGGCGCTCTGACATCCGGCGTCCACACCTTTCC |
| | | | | TGCCGTCCTGCAGTCCTCCGGCCTCTACTCCCTGTCCTCCGTGGTGACCGTGCCTAGC |
| | | | | TCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 549 | CL-58835 | Light chain | Nucleotide sequence of light | GAAATTGTGCTGACTCAGTCCCCTGGCACACTGTCTTTGAGCCCTGGCGAGAGAGCT |
| | | | chain alternative coding | ACCCTGTCTTGTTGGGCCTCTCAGAGAGTGGTGTACAGATACCTGGCTTGGTATCAG |
| | | | | CGGAAGCCCGGCCAGGCTCCTAGACTGTTGATCTACGGCGCCTTCAACAGAGCCAC |
| | | | | AGGCATCCCTGACAGATTCTCCGGCTCTGGCTCTGGCACCGACTTCTCCCTGACTATC |
| | | | | TCTCGGCTGGAACCCGAGGACTTCGCCGTGTACTACTGTCACCAGTACGGCAGCAGC |
| | | | | CCTCCTACCTTTGGCCAGGGCACTAAGGTGGAAATCAAACGTACGGTGGCCGCTCCC |
| | | | | TCCGTGTTCATCTTCCCACCTTCCGACGAGCAGCTGAAGTCCGGCACCGCTTCTGTCG |
| | | | | TGTGCCTGCTGAACAACTTCTACCCCCGCGAGGCCAAGGTGCAGTGGAAGGTGGAC |
| | | | | AACGCCCTGCAGTCCGGCAACTCCCAGGAATCCGTGACCGAGCAGGACTCCAAGGA |
| | | | | CAGCACCTACTCCCTGTCCTCCACCCTGACCCTGTCCAAGGCCGACTACGAGAAGCA |
| | | | | CAAGGTGTACGCCTGCGAAGTGACCCACCAGGGCCTGTCTAGCCCCGTGACCAAGT |
| | | | | CTTTCAACCGGGGCGAGTGT |
| 550 | CL-58756 | Heavy chain | Nucleotide sequence of heavy | CAGGTGCAGCTGGTGGAGTCTGGGGGAGGCTTGGTCAAGCCTGGAGGGTCCCTGA |
| | | | chain | GACTCTCCTGTGCAGCCTCTGGATTCACCTTCAGTGACTACTACATGAGCTGGATCCG |
| | | | | CCAGGCTCCAGGGAAGGGGCTGGAGTGGGTTTCATACATTAGTATTAGTGGTAGTA |
| | | | | CCATATACTACGCAGACTCTGTGAAGGGCCGATTCACCATCTCCAGGGACAACGCCA |
| | | | | AGAACTCACTGTATCTGCAAATGAACAGCCTGAGAGCCGAGGACACGGCCGTGTAT |
| | | | | TACTGTGCGAGAAGAAGCAGTGGCTGGTACGACTACTGGGGCCAGGGAACCCTGG |
| | | | | TCACCGTCTCCTCAGCCAGCACCAAGGGCCCTTCCGTGTTCCCCCTGGCCCCTTGCAG |
| | | | | CAGGAGCACCTCCGAATCCACAGCTGCCCTGGGCTGTCTGGTGAAGGACTACTTTCC |
| | | | | CGAGCCCGTGACCGTGAGCTGGAACAGCGGCGCTCTGACATCCGGCGTCCACACCT |
| | | | | TTCCTGCCGTCCTGCAGTCCTCCGGCCTCTACTCCCTGTCCTCCGTGGTGACCGTGCC |
| | | | | TAGCTCCTCCCTCGGCACCAAGACCTACACCTGTAACGTGGACCACAAACCCTCCAA |
| | | | | CACCAAGGTGGACAAACGGGTCGAGAGCAAGTACGGCCCTCCCTGCCCTCCTTGTCC |
| | | | | TGCCCCCGAGTTCGAAGGCGGACCCAGCGTGTTCCTGTTCCCTCCTAAGCCCAAGGA |
| | | | | CACCCTCATGATCAGCCGGACACCCGAGGTGACCTGCGTGGTGGTGGATGTGAGCC |
| | | | | AGGAGGACCCTGAGGTCCAGTTCAACTGGTATGTGGATGGCGTGGAGGTGCACAA |
| | | | | CGCCAAGACAAAGCCCCGGGAAGAGCAGTTCAACTCCACCTACAGGGTGGTCAGCG |
| | | | | TGCTGACCGTGCTGCATCAGGACTGGCTGAACGGCAAGGAGTACAAGTGCAAGTC |
| | | | | AGCAATAAGGGACTGCCCAGCAGCATCGAGAAGACCATCTCCAAGGCTAAAGGCCA |
| | | | | GCCCCGGGAACCTCAGGTGTACACCCTGCCTCCCAGCCAGGAGGAGATGACCAAGA |
| | | | | ACCAGGTGAGCCTGACCTGCCTGGTGAAGGGATTCTACCCTTCCGACATCGCCGTGG |
| | | | | AGTGGGAGTCCAACGGCCAGCCCGAGAACAATTATAAGACCACCCCTCCCGTCCTCG |
| | | | | ACAGCGACGGATCCTTCTTTCTGTACTCCAGGCTGACCGTGGATAAGTCCAGGTGGC |
| | | | | AGGAAGGCAACGTGTTCAGCTGCTCCGTGATGCACGAGGCCCTGCACAATCACTAC |
| | | | | ACCCAGAAGTCCCTGAGCCTGTCCCTGGGAAAG |
| 551 | CL-58756 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGCAGGGCCAGTCAGAGTGTTAGCAGCAACTACTTAGCCTGGTACCAG |
| | | | | CAGAAACCTGGCCAGGCTCCCAGGCTCCTCATCTATGGTGCATCCATCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGCTGTCACCAGTATGGTAGCTCA |
| | | | | CCTCCGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 552 | CL-58756 | Heavy chain | Nucleotide sequence of heavy | CAAGTTCAGTTGGTTGAGTCTGGCGGCGGACTGGTTAAGCCTGGCGGATCTCTGAG |
| | | | chain alternative coding | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCTCCACC |
| | | | | ATCTACTACGCCGACTCCGTGAAGGGCAGATTCACCATCTCCAGAGACAACGCCAAG |
| | | | | AACTCCCTGTACCTGCAGATGAACAGCCTGAGAGCCGAGGACACCGCCGTGTACTA |
| | | | | CTGCGCCCGTAGATCCTCTGGATGGTACGACTATTGGGGCCAGGGCACCCTGGTCA |
| | | | | CAGTTTCTAGTGCCAGCACCAAGGGCCCTTCCGTGTTCCCCCTGGCCCCTTGCAGCA |
| | | | | GGAGCACCTCCGAATCCACAGCTGCCCTGGGCTGTCTGGTGAAGGACTACTTTCCCG |
| | | | | AGCCCGTGACCGTGAGCTGGAACAGCGGCGCTCTGACATCCGGCGTCCACACCTTTC |
| | | | | CTGCCGTCCTGCAGTCCTCCGGCCTCTACTCCCTGTCCTCCGTGGTGACCGTGCCTAG |
| | | | | CTCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 553 | CL-58756 | Light chain | Nucleotide sequence of light | GAAATTGTGCTGACTCAGTCCCCTGGCACACTGTCTTTGAGCCCTGGCGAGAGAGCT |
| | | | chain alternative coding | ACCCTGTCCTGTAGAGCCTCTCAGTCCGTGTCCTCCAACTACCTGGCCTGGTATCAGC |
| | | | | AGAAGCCTGGACAGGCTCCCCGGCTGTTGATCTACGGCGCTTCTATCAGAGCCACA |
| | | | | GGCATCCCTGACCGGTTCTCCGGATCTGGCTCTGGCACCGATTTCACCCTGACCATCT |
| | | | | CTCGGCTGGAACCCGAGGATTTCGCCGTGTACTGCTGTCACCAGTACGGCTCTAGCC |
| | | | | CTCCTACCTTTGGACAGGGCACCAAGGTGGAAATCAAACGTACGGTGGCCGCTCCCT |
| | | | | CCGTGTTCATCTTCCCACCTTCCGACGAGCAGCTGAAGTCCGGCACCGCTTCTGTCGT |
| | | | | GTGCCTGCTGAACAACTTCTACCCCCGCGAGGCCAAGGTGCAGTGGAAGGTGGACA |
| | | | | ACGCCCTGCAGTCCGGCAACTCCCAGGAATCCGTGACCGAGCAGGACTCCAAGGAC |
| | | | | AGCACCTACTCCCTGTCCTCCACCCTGACCCTGTCCAAGGCCGACTACGAGAAGCAC |
| | | | | AAGGTGTACGCCTGCGAAGTGACCCACCAGGGCCTGTCTAGCCCCGTGACCAAGTC |
| | | | | TTTCAACCGGGGCGAGTGT |
| 554 | CL-58650 | Heavy chain | Nucleotide sequence of heavy | CAGGTGCAGCTGGTGGAGTCTGGGGGAGGCTTGGTCAAGCCTGGAGGGTCCCTGC |
| | | | chain | GACTCTCCTGTGCAGCCTCTGGATTCACCTTCAGTGACTACTTCATGAGCTGGATCCG |
| | | | | CCAGGCTCCAGGGAAGGGGCTGGAGTGGGTTTCGTACATTAGTATTAGTGGTAGTA |
| | | | | CCATATACTACGCAGACTCTGTGAAGGGCCGATTCACCATCTCCAGGGACAACGCCA |
| | | | | GGAACTCACTGTTTCTGCAAATGAACAGCCTGAGAGCCGAGGACACGGCCATCTATT |
| | | | | ACTGTGCGAGAAGAACCAGTGGCTGGTACGACTTCTGGGGCCAGGGAACCCTGGTC |
| | | | | ACCGTCTCCTCAGCCAGCACCAAGGGCCCTTCCGTGTTCCCCCTGGCCCCTTGCAGCA |
| | | | | GGAGCACCTCCGAATCCACAGCTGCCCTGGGCTGTCTGGTGAAGGACTACTTTCCCG |
| | | | | AGCCCGTGACCGTGAGCTGGAACAGCGGCGCTCTGACATCCGGCGTCCACACCTTTC |
| | | | | CTGCCGTCCTGCAGTCCTCCGGCCTCTACTCCCTGTCCTCCGTGGTGACCGTGCCTAG |
| | | | | CTCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 555 | CL-58650 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGCAGGGCCAGTCAGAGTGTTAGCTACAGCTACTTAGCCTGGTACCAG |
| | | | | CAGAAACCTGGCCAGGCTCCCAGGCTCCTCATCTATGGTGCATCCAGCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGTTGTCACCAGTATGGTAGTTCA |
| | | | | CCTCCGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 556 | CL-58679 | Heavy chain | Nucleotide sequence of heavy | CAGGTGCAGCTGGTGGAGTCTGGGGGAGGCTTGGTCAAGCCTGGAGGGTCCCTTA |
| | | | chain | GACTCTCCTGTGCAGCCTCTGGATTCACCTTCAGTGACTTCTACATGAGCTGGATCCG |
| | | | | CCAGGCTCCAGGGAGGGGGCTGGAGTGGGTTTCGTACATTAGTATTAGTGGTACTA |
| | | | | CCATATACTACGCAGACTCTGTGAAGGGCCGATTCACCATCTCCAGGGACAACGCCA |
| | | | | GGAACTCACTGTATCTGCAAATGAACAGCCTGAGAGCCGAGGACACGGCCGTGTAT |
| | | | | TACTGTGCGAGAAGAACCAGTGGCTGGTACGACTTCTGGGGCCAGGGAACCCTGGT |
| | | | | CACCGTCTCCTCAGCCAGCACCAAGGGCCCTTCCGTGTTCCCCCTGGCCCCTTGCAGC |
| | | | | AGGAGCACCTCCGAATCCACAGCTGCCCTGGGCTGTCTGGTGAAGGACTACTTTCCC |
| | | | | GAGCCCGTGACCGTGAGCTGGAACAGCGGCGCTCTGACATCCGGCGTCCACACCTT |
| | | | | TCCTGCCGTCCTGCAGTCCTCCGGCCTCTACTCCCTGTCCTCCGTGGTGACCGTGCCT |
| | | | | AGCTCCTCCCTCGGCACCAAGACCTACACCTGTAACGTGGACCACAAACCCTCCAAC |
| | | | | ACCAAGGTGGACAAACGGGTCGAGAGCAAGTACGGCCCTCCCTGCCCTCCTTGTCCT |
| | | | | GCCCCCGAGTTCGAAGGCGGACCCAGCGTGTTCCTGTTCCCTCCTAAGCCCAAGGAC |
| | | | | ACCCTCATGATCAGCCGGACACCCGAGGTGACCTGCGTGGTGGTGGATGTGAGCCA |
| | | | | GGAGGACCCTGAGGTCCAGTTCAACTGGTATGTGGATGGCGTGGAGGTGCACAAC |
| | | | | GCCAAGACAAAGCCCCGGGAAGAGCAGTTCAACTCCACCTACAGGGTGGTCAGCGT |
| | | | | GCTGACCGTGCTGCATCAGGACTGGCTGAACGGCAAGGAGTACAAGTGCAAGGTCA |
| | | | | GCAATAAGGGACTGCCCAGCAGCATCGAGAAGACCATCTCCAAGGCTAAAGGCCAG |
| | | | | CCCCGGGAACCTCAGGTGTACACCCTGCCTCCCAGCCAGGAGGAGATGACCAGAA |
| | | | | CCAGGTGAGCCTGACCTGCCTGGTGAAGGGATTCTACCCTTCCGACATCGCCGTGGA |
| | | | | GTGGGAGTCCAACGGCCAGCCCGAGAACAATTATAAGACCACCCCTCCCGTCCTCGA |
| | | | | CAGCGACGGATCCTTCTTTCTGTACTCCAGGCTGACCGTGGATAAGTCCAGGTGGCA |
| | | | | GGAAGGCAACGTGTTCAGCTGCTCCGTGATGCACGAGGCCCTGCACAATCACTACA |
| | | | | CCCAGAAGTCCCTGAGCCTGTCCCTGGGAAAG |
| 557 | CL-58679 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGCAGGGCCAGTCAGAGTGTTAGCTACAGCTACTTAGCCTGGTACCAG |
| | | | | CAGAAACCTGGCCAGGCTCCCAGGCTCCTCATCTATGGTGCATCCAGCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGCTGTCACCAGTATGGTAGTTCA |
| | | | | CCTCCGACGTTCGGCCAAGGGACCAAGGTGGAAATGAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 558 | CL-58680 | Heavy chain | Nucleotide sequence of heavy | CAGGTGCAGCTGGTGGAGTCTGGGGGAGGCTTGGTCAGGCCTGGAGGGTCCCTGA |
| | | | chain | GACTCTCCTGTGCAGCCTCTGGATTCACCTTCAGTGACTACTACATGAGCTGGATCCG |
| | | | | CCAGGCTCCAGGGAAGGGGCTGGAGTGGGTTTCGTACATTAGTATTAGTGGTAGTA |
| | | | | CCATATACTACGCAGACTCTGTGAAGGGCCGATTCACCATCTCCAGGGACAACGCCA |
| | | | | GGGACTCACTTTATCTGCAAATGAACAGCCTGAGAGCCGAGGACACGGCCGTGTAT |
| | | | | TACTGTGCGAGAAGAACCAGTGGCTGGTACGACTTCTGGGGCCAGGGAACCCTGGT |
| | | | | CACCGTCTCCTCAGCCAGCACCAAGGGCCCTTCCGTGTTCCCCCTGGCCCCTTGCAGC |
| | | | | AGGAGCACCTCCGAATCCACAGCTGCCCTGGGCTGTCTGGTGAAGGACTACTTTCCC |
| | | | | GAGCCCGTGACCGTGAGCTGGAACAGCGGCGCTCTGACATCCGGCGTCCACACCTT |
| | | | | TCCTGCCGTCCTGCAGTCCTCCGGCCTCTACTCCCTGTCCTCCGTGGTGACCGTGCCT |
| | | | | AGCTCCTCCCTCGGCACCAAGACCTACACCTGTAACGTGGACCACAAACCCTCCAAC |
| | | | | ACCAAGGTGGACAAACGGGTCGAGAGCAAGTACGGCCCTCCCTGCCCTCCTTGTCCT |
| | | | | GCCCCCGAGTTCGAAGGCGGACCCAGCGTGTTCCTGTTCCCTCCTAAGCCCAAGGAC |
| | | | | ACCCTCATGATCAGCCGGACACCCGAGGTGACCTGCGTGGTGGTGGATGTGAGCCA |
| | | | | GGAGGACCCTGAGGTCCAGTTCAACTGGTATGTGGATGGCGTGGAGGTGCACAAC |
| | | | | GCCAAGACAAAGCCCCGGGAAGAGCAGTTCAACTCCACCTACAGGGTGGTCAGCGT |
| | | | | GCTGACCGTGCTGCATCAGGACTGGCTGAACGGCAAGGAGTACAAGTGCAAGGTCA |
| | | | | GCAATAAGGGACTGCCCAGCAGCATCGAGAAGACCATCTCCAAGGCTAAAGGCCAG |
| | | | | CCCCGGGAACCTCAGGTGTACACCCTGCCTCCCAGCCAGGAGGAGATGACCAAGAA |
| | | | | CCAGGTGAGCCTGACCTGCCTGGTGAAGGGATTCTACCCTTCCGACATCGCCGTGGA |
| | | | | GTGGGAGTCCAACGGCCAGCCCGAGAACAATTATAAGACCACCCCTCCCGTCCTCGA |
| | | | | CAGCGACGGATCCTTCTTTCTGTACTCCAGGCTGACCGTGGATAAGTCCAGGTGGCA |
| | | | | GGAAGGCAACGTGTTCAGCTGCTCCGTGATGCACGAGGCCCTGCACAATCACTACA |
| | | | | CCCAGAAGTCCCTGAGCCTGTCCCTGGGAAAG |
| 559 | CL-58680 | Light chain | Nucleotide sequence of light | GAAATTGTGTTGACGCAGTCTCCAGGCACCCTGTCTTTGTCTCCAGGGGAAAGAGCC |
| | | | chain | ACCCTCTCCTGTAGGGCCAGTCAGAGTGTTAGCTACAGGTACTTAGCCTGGTACCAG |
| | | | | CAGAAACCTGGCCAGGCTCCCAGGCTCCTCATCTATGGTGCATCCAGCAGGGCCACT |
| | | | | GGCATCCCAGACAGGTTCAGTGGCAGTGGGTCTGGGACAGACTTCACTCTCACCATC |
| | | | | AGCAGACTGGAGCCTGAAGATTTTGCAGTGTATTGCTGTCACCAGTATGGTAGTTCA |
| | | | | CCTCCGACGTTCGGCCAAGGGACCAAGGTGGAAATCAAACGTACGGTGGCCGCTCC |
| | | | | CTCCGTGTTCATCTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGTGGAAGGTGGA |
| | | | | CAACGCCCTGCAGTCCGGCAACTCCCAGGAATCCGTGACCGAGCAGGACTCCAAGG |
| | | | | ACAGCACCTACTCCCTGTCCTCCACCCTGACCCTGTCCAAGGCCGACTACGAGAAGC |
| | | | | ACAAGGTGTACGCCTGCGAAGTGACCCACCAGGGCCTGTCTAGCCCCGTGACCAAG |
| | | | | TCTTTCAACCGGGGCGAGTGT |
| 560 | CL-58713 | Heavy chain | Nucleotide sequence of heavy | CAAGTTCAGTTGGTTGAGTCTGGCGGCGGACTGGTTAAGCCTGGCGGATCTCTGAG |
| | | | chain | ACTGTCTTGTGCCGCCTCTGGCTTCACCTTCTCCGACTACTACATGTCCTGGATCAGA |
| | | | | CAGGCCCCTGGCAAAGGCCTGGAATGGGTGTCCTACATCTCCATCTCCGGCATCACC |
| | | | | ATCTACTACGCCGACTCCGTGAAGGGCAGATTCACCATCTCCAGAGACAACGCCCGG |
| | | | | AACTCCCTGTACCTGCAGATGAACTCTCTGAGAGCCGAGGACACCGCCGTGTACTAC |
| | | | | TGCGCCCGTAGATCCTCTGGATGGTACGACTATTGGGGCCAGGGCACCCTGGTCAC |
| | | | | AGTTTCTAGTGCCAGCACCAAGGGCCCTTCCGTGTTCCCCCTGGCCCCTTGCAGCAG |
| | | | | GAGCACCTCCGAATCCACAGCTGCCCTGGGCTGTCTGGTGAAGGACTACTTTCCCGA |
| | | | | GCCCGTGACCGTGAGCTGGAACAGCGGCGCTCTGACATCCGGCGTCCACACCTTTCC |
| | | | | TGCCGTCCTGCAGTCCTCCGGCCTCTACTCCCTGTCCTCCGTGGTGACCGTGCCTAGC |
| | | | | TCCTCCCTCGGCACCAAGACCTACACCTGTAACGTGGACCACAAACCCTCCAACACC |
| | | | | AAGGTGGACAAACGGGTCGAGAGCAAGTACGGCCCTCCCTGCCCTCCTTGTCCTGC |
| | | | | CCCCGAGTTCGAAGGCGGACCCAGCGTGTTCCTGTTCCCTCCTAAGCCCAAGGACAC |
| | | | | CCTCATGATCAGCCGGACACCCGAGGTGACCTGCGTGGTGGTGGATGTGAGCCAGG |
| | | | | AGGACCCTGAGGTCCAGTTCAACTGGTATGTGGATGGCGTGGAGGTGCACAACGCC |
| | | | | AAGACAAAGCCCCGGGAAGAGCAGTTCAACTCCACCTACAGGGTGGTCAGCGTGCT |
| | | | | GACCGTGCTGCATCAGGACTGGCTGAACGGCAAGGAGTACAAGTGCAAGGTCAGC |
| | | | | AATAAGGGACTGCCCAGCAGCATCGAGAAGACCATCTCCAAGGCTAAAGGCCAGCC |
| | | | | CCGGGAACCTCAGGTGTACACCCTGCCTCCCAGCCAGGAGGAGATGACCAAGAACC |
| | | | | AGGTGAGCCTGACCTGCCTGGTGAAGGGATTCTACCCTTCCGACATCGCCGTGGAG |
| | | | | TGGGAGTCCAACGGCCAGCCCGAGAACAATTATAAGACCACCCCTCCCGTCCTCGAC |
| | | | | AGCGACGGATCCTTCTTTCTGTACTCCAGGCTGACCGTGGATAAGTCCAGGTGGCAG |
| | | | | GAAGGCAACGTGTTCAGCTGCTCCGTGATGCACGAGGCCCTGCACAATCACTACACC |
| | | | | CAGAAGTCCCTGAGCCTGTCCCTGGGAAAG |
| 561 | CL-58713 | Light chain | Nucleotide sequence of light | GAAATTGTGCTGACTCAGTCCCCTGGCACACTGTCTTTGAGCCCTGGCGAGAGAGCT |
| | | | chain | ACCCTGTCCTGTAGAGCCTCTCAGTCCGTGTCCTACAACTACCTGGCCTGGTATCAGC |
| | | | | AGAAGCCCGGCCAGGCTCCTAGACTGTTGATCTACGGCGCCTCCATCAGAGCCACA |
| | | | | GGCATCCCTGATAGATTCTCCGGCTCTGGCTCTGGCACCGACTTCACCCTGACCATCT |
| | | | | CCAGACTGGAACCCGAGGACTTCGCCGTGTACTGCTGTCACCAGTACGGCTCTAGCC |
| | | | | CTCCTACCTTTGGACAGGGCACCAAGGTGGAAATCAAACGTACGGTGGCCGCTCCCT |
| | | | | CCGTGTTCATCTTCCCACCTTCCGACGAGCAGCTGAAGTCCGGCACCGCTTCTGTCGT |
| | | | | GTGCCTGCTGAACAACTTCTACCCCCGCGAGGCCAAGGTGCAGTGGAAGGTGGACA |
| | | | | ACGCCCTGCAGTCCGGCAACTCCCAGGAATCCGTGACCGAGCAGGACTCCAAGGAC |
| | | | | AGCACCTACTCCCTGTCCTCCACCCTGACCCTGTCCAAGGCCGACTACGAGAAGCAC |
| | | | | AAGGTGTACGCCTGCGAAGTGACCCACCAGGGCCTGTCTAGCCCCGTGACCAAGTC |
| | | | | TTTCAACCGGGGCGAGTGT |
| 562 | Mature | | Amino acid sequence of a | SASSRRRQQSRNRSTQSQDVARVSSASDYNSSELKTACRKHELYVSFQDLGWQDWIIAP |
| | Human | | mature human BMP6 | KGYAANYCDGECSFPLNAHMNATNHAIVQTLVHLMNPEYVPKPCCAPTKLNAISVLYF |
| | BMP6 | | | DDNSNVILKKYRNMVVRACGCH |
| 563 | Mature Rat | | Amino acid sequence of a | SASSRRRQQSRNRSTQSQDVSRGSSASDYNSSELKTACKKHELYVSFQDLGWQDWIIAP |
| | BMP6 | | mature human BMP6 | KGYAANYCDGECSFPLNAHMNATNHAIVQTLVHLMNPEYVPKPCCAPTKLNAISVLYF |
| | | | | DDNSNVILKKYRNMVVRACGCH |
| 564 | Mature | | Amino acid sequence of a | SASGRRRQQSRNRSTQSQDVARVSSASDYNSSELKTACRKHELYVSFQDLGWQDWIIA |
| | Cynomolgus | | mature Cynomolgus monkey | PKGYAANYCDGECSFPLNAHMNATNHAIVQTLVHLMNPEYVPKPCCAPTKLNAISVLYF |
| | monkey | | BMP6 | DDNSNVILKKYRNMVVRACGCH |
| | BMP6 | | | |
| 565 | Antibody A′ | Heavy chain | Amino acid sequence of VH | QVQLVQSGAEVKKPGSSVKVSCKASGYTFTSYAMHWVRQAPGQGLEWMGYINPYNR |
| | | variable | | GTKYNENFKGRVTITADESTSTAYMELSSLRSEDTAVYYCARRPFGNAMDIWGQGTLVT |
| | | region | | VSS |
| 566 | Antibody A′ | Full heavy | Amino acid sequence heavy | QVQLVQSGAEVKKPGSSVKVSCKASGYTFTSYAMHWVRQAPGQGLEWMGYINPYNR |
| | | chain | chain of Antibody A′ | GTKYNENFKGRVTITADESTSTAYMELSSLRSEDTAVYYCARRPFGNAMDIWGQGTLVT |
| | | sequence | | VSSASTKGPSVFPLAPCSRSTSESTAALGCLVKDYFPEPVTVSWNSGALTSGVHTFPAVL |
| | | | | QSSGLYSLSSVVTVPSSSLGTKTYTCNVDHKPSNTKVDKRVESKYGPPCPPCPAPEFEGG |
| | | | | PSVFLFPPKPKDTLMISRTPEVTCVVVDVSQEDPEVQFNWYVDGVEVHNAKTKPREEQ |
| | | | | FNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKS |
| | | | | RWQEGNVFSCSVMHEALHNHYTQKSLSLSLGK |

1. An antibody or fragment comprising a binding site which specifically binds to Bone Morphogenetic Protein 6 (BMP6), wherein the binding site comprises a VH domain, wherein the VH domain comprises a CDRH3 sequence of a VH domain comprising SEQ ID NO: 114.

2. The antibody of claim 1, wherein the CDRH3 comprises SEQ ID NO: 110 or 113.

3. The antibody of any preceding claim wherein the VH domain is encoded by a nucleotide sequence that is derived from the recombination of a human VH gene segment, DH gene segment and JH gene segment, wherein the
(a) VH gene segment is selected from IGHV3-11 and IGHV1-3;
(b) the DH gene segment is a human gene segment selected from IGHD3-10, IGHD6-19, IGHD7-27, IGHD4-23, IGHD5-18, IGHD3-22 and IGHD3-16; and/or
(c) the JH gene segment is a human gene segment selected from IGHJ3, IGHJ4 and IGHJ5.

4. The antibody or fragment according to any preceding claim, wherein the binding site comprises (i) a VH domain comprising SEQ ID NO: 110 paired with a VL domain comprising SEQ ID NO: 119; or (ii) a VH domain comprising SEQ ID NO: 113 paired with a VL domain comprising SEQ ID NO: 122.

5. The antibody or fragment according to any preceding claim, wherein the binding site comprises a VH domain comprising SEQ ID NO: 114 paired with a VL domain comprising SEQ ID NO: 123.

6. The antibody or fragment according to any preceding claim, wherein the VH domain comprises
(a) a CDRH3 comprising SEQ ID NO: 110; and a CDRH1 comprising SEQ ID NO: 108 and/or a CDRH2 comprising SEQ ID NO: 109; or
(b) a CDRH3 comprising SEQ ID NO: 113; and a CDRH1 comprising SEQ ID NO: 111 and/or a CDRH2 comprising SEQ ID NO: 112.

7. An antibody or fragment (optionally according to any preceding claim) comprising a binding site which specifically binds to BMP6, wherein the binding site comprises a VH domain that comprises SEQ ID NO: 114, or an amino acid that is at least 70% identical thereto.

8. The antibody or fragment according to any preceding claim comprising first and second copies of said VH domain.

9. An antibody or fragment (optionally according to any preceding claim) comprising a binding site which specifically binds to BMP6, wherein the binding site comprises a VL domain, wherein the VL domain comprises a CDRL3 sequence of a VL domain comprising SEQ ID NO: 123.

10. The antibody of claim 9, wherein the CDRL3 comprises SEQ ID NO: 119 or 122.

11. The antibody of claim 9 or 10, wherein the VL domain is encoded by a nucleotide sequence that is derived from the recombination of a human VL gene segment and JL gene segment, wherein
(a) the VL gene segment is selected from IGKV3-20, IGKV1-5 and IGKV3-15; and/or
(b) the JL gene segment is selected from IGKJ1 and IGKJ3.

12. The antibody or fragment according to any one of claims 9 to 11, wherein the VL domain comprises
(a) a CDRL3 comprising SEQ ID NO: 119; and a CDRL1 comprising SEQ ID NO: 117 and/or a CDRL2 comprising the amino acid sequence GAF; or
(b) a CDRL3 comprising SEQ ID NO: 122; and a CDRL1 comprising SEQ ID NO: 120 and/or a CDRL2 comprising SEQ ID NO: 121.

13. An antibody or fragment (optionally according to any preceding claim) comprising a binding site which specifically binds to BMP6, wherein the binding site comprises a VL domain that comprises SEQ ID NO: 123, or an amino acid that is at least 70% identical thereto.

14. The antibody or fragment according to any one of claims 9 to 13 comprising first and second copies of said VL domain.

15. An antibody or fragment (optionally according to any preceding claim) which specifically binds to Bone Morphogenetic Protein 6 (BMP6) and comprises (i) a heavy chain amino acid sequence comprising SEQ ID NO: 116 or an amino acid that is at least 70% identical thereto; and/or (ii) a light chain sequence comprising SEQ ID NO: 125 or an amino acid that is at least 70% identical thereto.

16. An antibody or fragment (optionally according to any preceding claim) which (i) specifically binds to a human BMP6 epitope that is identical to an epitope to which the antibody of any preceding claim binds; and/or (ii) competes for binding to human BMP6 with the antibody of any preceding claim.

17. The antibody or fragment according to any preceding claim which specifically binds to human BMP6 comprising SEQ ID NO: 562; and/or a cynomolgus BMP6 comprising SEQ ID NO: 564; and/or a rat BMP6 comprising SEQ ID NO: 563.

18. The antibody or fragment according to any preceding claim, wherein the antibody or fragment comprises a human constant region, optionally an IgG4 constant region or an IgG1 constant region.

19. The antibody or fragment according to claim 18, wherein the constant region is an IgG4-PE constant region, optionally the constant region comprises the amino acid sequence of SEQ ID NO: 454.

20. The antibody or fragment according to any preceding claim further comprising an antigen-binding site that specifically binds another target antigen (optionally human hemojuvelin, transferrin receptor or a BMP receptor); or binds BMP6 and another BMP (optionally BMP2, 4, 7 or 9).

21. An anti-BMP6 antibody or fragment as defined in any preceding claim for treating or preventing a BMP6-mediated disease or condition (optionally anaemia) in a subject.
